(19) United States
(12) Patent Application Publication
(10) Pub. No.: US 2024/0254182 A1
(43) Pub. Date: Aug. 1, 2024

(54) MODIFIED GRANULOCYTE COLONY-STIMULATING FACTOR (G-CSF) AND CHIMERIC CYTOKINE RECEPTORS BINDING SAME

(71) Applicants: Provincial Health Services Authority, Vancouver (CA); UVIC Industry Partnerships Inc., Victoria (CA)

(72) Inventors: Martin J. Boulanger, Victoria (CA); Brad Nelson, Victoria (CA); Cameron Powell, Victoria (CA); Megan Fuller, Victoria (CA)

(21) Appl. No.: 18/286,045

(22) PCT Filed: Apr. 7, 2022
(86) PCT No.: PCT/CA2022/050540

§ 371 Date: Oct. 6, 2023

Related U.S. Application Data
(60) Provisional application No. 63/171,933, filed on Apr. 7, 2021.
(60) Provisional application No. 63/171,950, filed on Apr. 7, 2021.
(60) Provisional application No. 63/171,980, filed on Apr. 7, 2021.
(60) Provisional application No. 63/172,025, filed on Apr. 7, 2021.

(51) Int. Cl. C07K 14/535; A61K 38/00; A61K 38/20; A61K 39/00; C07K 14/705; C07K 14/715; C12N 5/0783; C12N 15/86

(52) U.S. Cl. CPC C07K 14/535; A61K 38/2046; A61K 39/4637; C07K 14/70503; C07K 14/7153; C07K 14/7155; C12N 5/0636; C12N 15/86; A61K 38/00; C12N 2740/15043

(57) ABSTRACT

Described herein are methods and compositions for selective activation of cells using variant cytokine receptor and cytokine pairs, wherein the cytokine receptors comprise an extracellular domain (ECD) of granulocyte-colony stimulating factor receptor (G-CSFR). In certain embodiments, the methods and compositions described herein are useful for exclusive activation of cells for adoptive cell transfer therapy. Thus, included herein are methods of producing cells expressing variant receptors that are selectively activated by a cytokine that does not bind its native receptor. Also disclosed herein are methods of treating a subject in need thereof, comprising administering to the subject cells expressing an variant receptor comprising an extracellular domain of G-CSFR and co-administering a variant cytokine that activates the variant receptor.

### CROSS REFERENCE TO RELATED APPLICATIONS

This application is the National Stage of International Application No. PCT/CA2022/050540, filed Apr. 7, 2022, which claims the benefit of U.S. Provisional Application Nos. 63/171,933 filed Apr. 7, 2021; 63/171,950 filed Apr. 7, 2021; 63/171,980 filed Apr. 7, 2021 and 63/172,025 filed Apr. 7, 2021, each of which is hereby incorporated in its entirety by reference for all purposes.

### SEQUENCE LISTING

The instant application contains a Sequence Listing which has been submitted via EFSWeb and is hereby incorporated by reference in its entirety. Said ASCII copy, created on Feb. 22, 2024, is named IKE-002WO-143520322444_ST25.txt, and is 302,426 bytes in size.

### BACKGROUND

### Field

In certain aspects, described herein are methods and compositions for selective activation of cells using variant cytokine receptor and cytokine pairs, wherein the cytokine receptors comprise a variant extracellular domain (ECD) of granulocyte-colony stimulating factor receptor (G-CSFR). Also disclosed herein are methods of treating a subject by adoptive cell transfer, comprising administering to the subject cells expressing variant receptors and administering variant cytokines to send signals to the cells expressing variant receptors. Included with this disclosure are nucleic acids, expression vectors and kits for producing cells expressing variant cytokines and receptors, and kits which also provide cytokines for binding variant receptors. In certain aspects, described herein are chimeric cytokine receptors comprising G-CSFR (Granulocyte-Colony Stimulating Factor Receptor) extracellular domains and the intracellular domains of various cytokine receptors for selective activation of cytokine signaling in cells of interest. The present disclosure also comprises methods, cells and kits for use in adoptive cell transfer (ACT), comprising cells expressing the chimeric cytokine receptors and/or expression vectors encoding chimeric cytokine receptors and/or cytokines that bind the chimeric cytokine receptors. In certain aspects, described herein are systems and methods for controlled paracrine signaling comprising chimeric cytokine receptors comprising G-CSFR (Granulocyte-Colony Stimulating Factor Receptor) extracellular domains and the intracellular domains of various cytokine receptors for selective activation of cytokine signaling in cells of interest.

Also described herein are methods and compositions for selective activation of cells using variant cytokine receptor and cytokine pairs, wherein the cytokine receptors comprise an extracellular domain (ECD) of Interleukin-7 Receptor Alpha (IL-7Rα).

The past two decades have brought much progress in the treatment of cancer by adoptive cell transfer (ACT). ACT with naturally occurring tumor-infiltrating T cells (TIL) is now reproducibly yielding >50% objective clinical response rates in advanced melanoma. ACT with T cells engineered to recognize B-lineage leukemias (using CD19-directed chimeric antigen receptors, or CD19 CARs) is yielding up to 90% complete response rates, with the majority of patients achieving durable responses. ACT with T cells expressing engineered T Cell Receptors (TCRs) is showing promise against various solid tumors. Successful ACT has also been reported using other effector cell types in place of T cells, including Natural Killer (NK) cells, Natural Killer T cells (NKT cells) and macrophages. Motivated by these remarkable results, several companies are commercializing the TIL, CAR and engineered TCR ACT approaches.

The engraftment, expansion and persistence of T cells or other effector cells for ACT are important determinants of clinical safety and efficacy. In the case of T cells, this is often addressed by administration of systemic IL-2 after ACT transfer, as well as expanding the T cells outside the body with IL-2 prior to transfer. In addition to the intended immune stimulatory effects, systemic IL-2 treatment can also lead to severe toxicities such as vascular leakage syndrome that need to be tightly controlled for patient safety. To manage these risks, patients typically need to be hospitalized for 2-3 weeks and have access to an ICU as a precautionary measure. Furthermore, IL-2 induces the proliferation of both effector and regulatory (inhibitory) T cells (5); therefore, giving a patient IL-2 is analogous to pressing the gas and brake pedals at the same time. CAR T cells bring the converse problem in that T-cell expansion and persistence exceed safe levels in some patients, and falter prematurely in others. Moreover, they universally eradicate normal B cells (which also express CD19), leaving patients partly immune-deficient. Ideally, one would like to have precise control over the number of tumor-reactive T cells after ACT, including the abilities to safely enhance the proliferation, persistence and potency of T cells and to eliminate transferred cells once the cancer has been eradicated. Other cell-based therapies, such as stem cell therapies, would also benefit from improved control over the expansion, differentiation, and persistence of infused cells.

Human G-CSF (Neupogen®, Filgrastim) and a pegylated version of human G-CSF (Neulasta®, Pegfilgrastim) are approved therapeutics used to treat Neutropenia in cancer patients. G-CSF is a four-helix bundle (Hill, C P et al. Proc Natl Acad Sci USA. 1993 Jun. 1; 90(11):5167-71), and the structure of G-CSF in complex with its receptor G-CSFR is well characterized (Tamada, T et al. Proc Natl Acad Sci USA. 2006 Feb. 28; 103(9):3135-40). The G-CSF:G-CSFR complex is a 2:2 heterodimer. G-CSF has two binding interfaces with G-CSFR. One interface is referred to as site II; it is the larger interface between G-CSF and the Cytokine Receptor Homologous (CRH) domain of G-CSFR. The second interface is referred to as site III; it is the smaller interface between G-CSF and the N-terminal Ig-like domain of G-CSFR.

Interleukin 7 (IL-7) is an example of safe, well-tolerated cytokine but with limited potency in the setting of cancer immunotherapy. IL-7 is a growth factor for T cells and B cells and is important for thymic development and supporting the survival and homeostasis of naïve and memory T cells. Unlike IL-2, IL-7 induces minimal Treg proliferation, as IL-7Rα is expressed at low levels on this suppressive lymphocyte population. IL-7 is not produced by hematopoietic cells but rather is secreted by stromal cells. IL-7 has been used in cancer immunotherapy with the goal of increasing T cell numbers, persistence and activity (Barata J T et al. Nat Immunol. 2019 December; 20(12):1584-1593). It has been shown to be well-tolerated; however, it has shown negligible anti-cancer efficacy as a monotherapy (Rosenberg et al. J Immunother. 2006 May-June; 29(3):313-9), (Sportes C. et al., Clin Cancer Res. 2010 Jan. 15; 16(2):727-35), and (Sportes C., et al, J Exp Med. 2008 Jul. 7; 205(7):1701-14). Moreover, recombinant IL-7 made in E. coli proved to be highly immunogenic, although this problem was ameliorated by producing IL-7 in mammalian cells (Conlon K C, et al., J Interferon Cytokine Res. 2019 January; 39(1):6-21). For these reasons, there has been limited clinical development of IL-7 in oncology relative to more therapeutically potent cytokines such as IL-2 and IL-15. However, IL-7 is attractive for use as a ligand for chimeric receptors that induce more potent intracellular signals than achieved by the native IL-7 receptor.

To mitigate the toxicity issues associated with systemic delivery of cytokines, several groups have engineered T cells or NK cells to produce and secrete cytokines in an autocrine/paracrine manner. The goal is to have the engineered T/NK cells produce enough cytokine for their own consumption, and that of neighboring cells, but not enough to cause systemic toxicities. For example, this strategy has been applied to improve Chimeric Antigen Receptor (CAR) T cell and CAR NK cell therapy using cytokines such as IL-2, IL-15, IL-12 and IL-18. A common approach is to use retroviruses or lentiviruses to stably introduce a CAR gene plus cytokine gene in a T cell or NK cell population. This can be achieved by a variety of approaches, such as co-transduction of two viral vectors, or by using a bicistronic viral vector that carries two transgenes. T cells and NK cells that co-express CARs and cytokine transgenes are commonly referred to as “armored CARs” or “TRUCKS”.

Armored CARs have been evaluated in murine tumor models and, to a lesser extent, in human clinical trials. In several studies, armored CAR T/NK cells have proven both safe and effective relative to standard CAR T/NK cells (i.e. cells with a CAR but no cytokine transgene). However, toxicities have been observed in some mouse studies (Ataca Atilla P., et al., J Immunother Cancer. 2020 September; 8(2):e001229) and clinical trials (Zhang L., et al., Clin Cancer Res. 2015 May 15; 21(10):2278-88. Moreover, there will always be theoretical concerns about the concept of endowing T/NK cells (or any cell type) with the ability to produce an autocrine growth factor; this has the potential to lead to uncontrolled T/NK cell growth, resulting in a secondary lymphoproliferative disease or malignancy. Some investigators attempt to mitigate this risk by co-expressing a “suicide gene” that can be used to kill the T/NK cells in response to a drug, should toxicities, uncontrolled growth or other concerns arise. However, deployment of a suicide gene by definition terminates the cell therapy, potentially leaving the patient with residual tumor burden. Therefore, novel compositions and methods are needed for reducing the toxicity and safety risks associated with the armored CAR approach to enable therapeutically relevant cytokine signals to be delivered to immune cells in a safe and controlled manner.

### SUMMARY

Described herein are variant Granulocyte Colony-Stimulating Factors (G-CSF), wherein the variant G-CSF comprises at least one mutation in a site II interface region, at least one mutation in a site III interface region, or combinations thereof; wherein the at least one mutation in the site II interface region is selected from the group of mutations consisting of: L108R, D112R, E122R E122K, E123K and E123R and combinations thereof; wherein the site II interface region mutations are relative to the corresponding amino acid positions of the sequence shown in SEQ ID NO. 1; wherein the at least one mutation in the site III interface region comprises mutation E46R relative to the corresponding amino acid positions of the sequence shown in SEQ ID NO. 1; and wherein the variant G-CSF binds selectively to a receptor comprising a variant extracellular domain (ECD) of Granulocyte Colony-Stimulating Factor Receptor (G-CSFR).

In certain aspects, described herein is a system for selective activation of a receptor expressed on a cell surface, the system comprising: (a) a variant G-CSF corresponding to SEQ ID NO: 83 or 84; and (b) a receptor comprising a variant ECD of G-CSFR; wherein the variant G-CSF preferentially binds the receptor comprising the variant ECD of G-CSFR as compared to an otherwise identical wild type G-CSFR ECD, and the receptor comprising the variant ECD of G-CSFR preferentially binds the variant G-CSF as compared to an otherwise identical wild type G-CSF; and wherein the variant G-CSFR comprises G2R-3 or G12/2R-1.

In some embodiments, the variant G-CSF binds a receptor comprising a variant ECD of G-CSFR that is expressed by a cell. In some embodiments, the cell expressing the receptor comprising the variant ECD of G-CSFR is an immune cell. In some embodiments, the immune cell expressing the receptor comprising the variant ECD of G-CSFR is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T-cell is selected from the group consisting of a CD8⁺ T cell, cytotoxic CD8⁺ T cell, naïve CD8⁺ T cell, naïve CD4⁺ T cell, helper T cell, regulatory T cell, memory T cell, and γδT cell.

In some embodiments, the selective binding of the variant G-CSF to the receptor comprising the variant ECD of G-CSFR causes a cellular response comprising at least one of proliferation, viability, persistence, cytotoxicity, cytokine secretion, memory, and enhanced activity of a cell expressing the receptor. In some embodiments, the receptor comprising the variant ECD of G-CSFR comprises at least one mutation in a site II interface region, at least one mutation in a site III interface region, or combinations thereof. In some embodiments, the receptor comprising the variant ECD of G-CSFR comprises at least one mutation in the site II interface region of the G-CSFR ECD comprising one or both of a R141E or a R167D mutation; wherein the at least one mutation in the site III interface region of the G-CSFR ECD comprises a R41E mutation; and wherein the variant G-CSFR mutations correspond to an amino acid position of the sequence shown in SEQ ID NO. 2. In some embodiments, the receptor comprising the variant ECD of G-CSFR is a chimeric receptor.

In certain aspects, the present disclosure describes one or more nucleic acid sequence(s) encoding the variant G-CSF described herein. In certain aspects, the present disclosure describes one or more expression vector(s) comprising the nucleic acid sequence. In certain aspects, the present disclosure describes cells engineered to express a variant G-CSF described herein. In some embodiments, the cell is an immune cell. In some embodiments, the immune cell is a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell, is selected from the group consisting of a CD8⁺ T cell, cytotoxic CD8⁺ T cell, naïve CD8⁺ T cell, naïve CD4⁺ T cell, helper T cell, regulatory T cell, memory T cell, and γδT cell.

In certain aspects, described herein is a system for selective activation of a receptor expressed on a cell surface, the system comprising: (a) the variant G-CSF described herein; and (b) a receptor comprising a variant ECD of G-CSFR; wherein the variant G-CSF preferentially binds the receptor comprising the variant ECD of G-CSFR as compared to an otherwise identical wild type G-CSFR ECD, and the receptor comprising the variant ECD of G-CSFR preferentially binds the variant G-CSF as compared to an otherwise identical wild type G-CSF. In some embodiments, the variant G-CSF comprises a combination of mutations of a site II and a site III interface of a G-CSF variant number of Table 4A; wherein the variant G-CSF mutations correspond to an amino acid position of the sequence shown in SEQ ID NO. 1.

In some embodiments, the variant G-CSF comprises mutations E46R, L108K, D112R, E122R, and E123R relative to the corresponding amino acid positions of the sequence shown in SEQ ID NO. 1; and wherein the receptor comprising a variant ECD of G-CSFR comprises mutations R41E, R141E and R167D relative to the corresponding amino acid positions of the sequence shown in SEQ ID NO. 2. In some embodiments, the variant G-CSF comprises mutations E46R, L108K, D112R, and E122K relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2. In some embodiments, the variant G-CSF comprises mutations E46R, L108K, D112R, and E123K relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2. In some embodiments, the variant G-CSF comprises mutations E46R, L108K, D112R, and E122R relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2. In some embodiments, the variant G-CSF comprises mutations E46R, L108K, D112R, and E123R relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2. In some embodiments, the variant G-CSF comprises mutations E46R, L108K, D112R, E122K, and E123K relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2.

In some embodiments, the system further comprises one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the system further comprises an antigen binding signaling receptor. In some embodiments, the antigen binding signaling receptor comprises at least one receptor selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, and a pattern recognition receptor. In some embodiments, the antigen binding signaling receptor is a CAR. In some embodiments, the cytokine or chemokine is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, and CCL19, and the receptor NKG2D. In some embodiments, the cytokine is IL-18. In some embodiments, the cytokine is human.

In certain aspects, described herein is a method of selective activation of a receptor expressed on the surface of a cell, comprising contacting a receptor comprising a variant ECD of G-CSFR with a variant G-CSF described herein. In some embodiments, the receptor comprising a variant ECD of G-CSFR is expressed on an immune cell, and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell is selected from the group consisting of CD8⁺ T cells, cytotoxic CD8⁺ T cells, naïve CD8⁺ T cells, naïve CD4⁺ T cells, helper T cells, regulatory T cells, memory T cells, and γδT cells. In some embodiments, the selective activation of the immune cell causes a cellular response comprising at least one of proliferation, viability, persistence, cytotoxicity, cytokine secretion, memory, and enhanced activity of a cell expressing the receptor.

In certain aspects, the present disclosure describes a method of increasing an immune response in a subject in need thereof, comprising: administering cell(s) expressing a receptor comprising a variant ECD of G-CSFR and administering or providing a variant G-CSF described herein. In certain aspects, the present disclosure describes a method of treating a disease in a subject in need thereof, comprising: administering cell(s) expressing a receptor comprising a variant ECD of G-CSFR and administering or providing a variant G-CSF described herein to the subject. In some embodiments, the method is used to treat cancer. In some embodiments, the method is used to treat an inflammatory condition. In some embodiments, the method is used to treat an autoimmune disease. In some embodiments, the method is used to treat a degenerative disease. In some embodiments, the method is used to generate natural or engineered cells, tissues or organs for transplantation. In some embodiments, the method is used to prevent or treat graft rejection. In some embodiments, the method is used to treat an infectious disease. In some embodiments, the methods further comprise administering or providing one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent.

In some embodiments, the subject is administered two or more populations of cells each expressing one or both of: (i) a distinct chimeric receptor comprising a G-CSFR ECD and (ii) at least one distinct variant form of G-CSF. wherein at least one population(s) of cells further express at least one distinct antigen binding signaling receptor(s); and, optionally, wherein the at least one distinct antigen binding signaling receptor(s) comprises at least one CAR(s). In some embodiments, one or both of the first and second population(s) of immune cells further expresses one or both of: (a)) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor. In some embodiments, the method further comprises one or more additional populations of immune cells; wherein each additional population of immune cells expresses at least one of (i) a distinct receptor comprising a distinct variant ECD of G-CSFR, (ii) a distinct variant G-CSF, (iii) a distinct an agonistic or antagonistic signaling protein and (iv) a distinct antigen binding signaling receptor.

In some embodiments of the methods, the cells expressing a receptor comprising a variant ECD of G-CSFR further express at least one antigen binding signaling receptor(s). In some embodiments, the antigen binding signaling receptor(s) comprises at least one receptor selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In some embodiments, the antigen binding signaling receptor(s) comprises one or more CAR(s). In some embodiments, the cytokine or chemokine is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, and CCL19, and the receptor NKG2D, and combinations thereof. In some embodiments, the cytokine is IL-18. In some embodiments, the cytokine is human.

In certain aspects, described herein are methods of treating a subject in need thereof, wherein the method comprises: i) isolating an immune cell-containing sample; (ii) transducing or transfecting the immune cell(s) with a nucleic acid sequence(s) encoding at least one receptor(s) comprising a variant ECD of G-CSFR; (iii) administering the immune cell(s) from (ii) to the subject; and (iv) contacting the immune cell(s) with one or more variant G-CSF described herein that selectively binds the receptor(s). In some embodiments, the subject has undergone an immuno-depletion treatment prior to administering the cells to the subject. In some embodiments, the immune cell-containing sample is isolated from the subject to whom the cells will be administered. In some embodiments, the immune cell-containing sample is generated from cells derived from the subject to whom the cells are administered or from a subject distinct from a subject to whom the cells will be administered, and, optionally, wherein the cells are stem cells, and, optionally, pluripotent stem cells. In some embodiments, the immune cell(s) are contacted with at least one variant G-CSF in vitro prior to administering the cells to the subject. In some embodiments, the immune cell(s) are contacted with the at least one variant G-CSF that binds the receptor(s) for a sufficient time to activate signaling from the receptor(s).

In certain aspects, described herein are kits comprising: cells encoding at least one receptor(s) comprising a variant ECD of G-CSFR and instructions for use; and wherein the kit comprises at least one variant G-CSF described herein; and, optionally, wherein the cells are immune cells. In certain aspects, described herein are kits comprising: (a) one or more nucleic acid sequence(s) encoding one or more receptor(s) comprising the variant ECD of G-CSFR; (b) a at least one variant G-CSF described herein, a nucleic acid sequence(s) described herein or one or more expression vector(s) described herein; and (c) instructions for use. In some embodiments, the kit further comprises one or more expression vector(s) that encode one or more cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, and CCL19, and the receptor NKG2D, and combinations thereof. In some embodiments, the kit further comprises one or more expression vector(s) that encodes at least one antigen binding receptor(s). In some embodiments, the at least one antigen binding receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In some embodiments, the kit further comprises one or more expression vector(s) that encodes a chimeric antigen receptor. In some embodiments, the cells further comprise one or more expression vector(s) that encode at least one cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, and CCL19, and the receptor NKG2D, and combinations thereof. In some embodiments, the cells further comprise one or more expression vector(s) that encode at least one antigen binding signaling receptor(s). In some embodiments, the at least one antigen binding signaling receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In some embodiments, the cells further comprise one or more expression vector(s) that encode at least one CAR(s), and, optionally the CAR is a mesothelin CAR.

In certain aspects, described herein are chimeric receptors, comprising: (a) an extracellular domain (ECD) operatively linked to at least one second domain; the second domain comprising: (b) an intracellular domain (ICD) comprising at least one signaling molecule binding site from an intracellular domain of a cytokine receptor; wherein the at least one signaling molecule binding site is selected from the group consisting of: a SHC binding site of Interleukin (IL)-2Rβ; a STAT5 binding site of IL-2Rβ, an IRS-1 or IRS-2 binding site of IL-4Rα, a STAT6 binding site of IL-4Rα, a SHP-2 binding site of gp130, a STAT3 binding site of gp130, a SHP-1 or SHP-2 binding site of EPOR, a STAT5 binding site of Erythropoietin Receptor (EPOR), a STAT1 or STAT2 binding site of Interferon Alpha And Beta Receptor Subunit 2 (IFNAR2), and a STAT1 binding site of Interferon Gamma Receptor 1 (IFNγR1), or combinations thereof; wherein the ICD further comprises at least one Box 1 region and at least one Box 2 region of at least one protein selected from the group consisting of G-CSFR, gp130, EPOR, and Interferon Gamma Receptor 2 (IFNγR2), or combinations thereof; and (c) at least one third domain comprising a transmembrane domain (TMD); wherein the ECD is N-terminal to the TMD, and the TMD is N-terminal to the ICD.

In certain aspects, described herein are chimeric receptors, comprising: an ECD operatively linked to a second domain; the second domain comprising an ICD, wherein the ICD comprises:
(i)
   (a) a Box 1 and a Box 2 region of G-CSFR;
   (b) at least one signaling molecule binding site of IL-4Rα; or
(ii)
   (a) a Box 1 and a Box 2 region of gp130;
   (b) at least one signaling molecule binding site of gp130; or
(iii)
   (a) a Box 1 and a Box 2 region of Erythropoietin Receptor (EPOR);
   (b) at least one signaling molecule binding site of EPOR; or
(iv)
   (a) a Box 1 and a Box 2 region of G-CSFR;
   (b) at least one signaling molecule binding site of Interferon Alpha and Beta Receptor Subunit 2 (IFNAR2); or
(v)
   (a) a Box 1 and a Box 2 region of Interferon Gamma Receptor 2 (IFNγR2);
   (b) at least one signaling molecule binding site of Interferon Gamma Receptor 1 (IFN γR1); wherein the ECD is N-terminal to a TMD, and the TMD is N-terminal to the ICD.

In certain embodiments, the ECD of the chimeric receptor is an ECD of G-CSFR (Granulocyte-Colony Stimulating Factor Receptor). In certain embodiments, the TMD of the chiomeric receptor is a TMD of G-CSFR and, optionally, the TMD is a wild-type TMD. In certain embodiments, an activated form of the chimeric receptor forms a homodimer, and, optionally, activation of the chimeric receptor causes a cellular response comprising at least one of proliferation, viability, persistence, cytotoxicity, cytokine secretion, memory, and enhanced activity of a cell expressing the receptor, and, optionally, the chimeric receptor is activated upon contact with a G-CSF, and, optionally, the G-CSF is a wild-type G-CSF, and, optionally, the extracellular domain of the G-CSFR is a wild-type extracellular domain.

In certain embodiments, the chimeric receptor is expressed in a cell, and, optionally, an immune cell, and, optionally the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In certain embodiments, the T cell is selected from the group consisting of a CD8⁺ T cell, cytotoxic CD8⁺ T cell, naïve CD4⁺ T cell, naïve CD8⁺ T cell, helper T cell, regulatory T cell, memory T cell, and γδT cell.

In certain embodiments, the ICD comprises:
(a) an amino acid sequence of one or both of SEQ ID NO. 90 or 91; or
(b) an amino acid sequence of one or both of SEQ ID NO. 90 or 92; or
(c) an amino acid sequence of SEQ ID NO. 93; or
(d) an amino acid sequence of SEQ ID NO. 94; or
(e) an amino acid sequence of one or both of SEQ ID NO. 95 or 96; or
(f) an amino acid sequence of SEQ ID NO. 97 or 98; or
(g) an amino acid sequence of SEQ ID NO. 99 or 100.

In certain embodiments, the transmembrane domain comprises a sequence set forth in SEQ ID NO. 88.

In certain aspects, this disclosure describes one or more nucleic acid sequence(s) encoding a chimeric receptor described herein. In certain embodiments, the nucleic acid sequence(s) of the ECD of the G-CSFR is encoded by nucleic acid sequence(s) set forth in any one of SEQ ID NO. 85, 86, or 87. In certain embodiments, this disclosure describes one or more expression vector(s) comprising the nucleic acid sequence(s). In certain embodiments, the expression vector(s) are selected from the group consisting of: a retroviral vector, a lentiviral vector, an adenoviral vector and a plasmid.

In certain aspects, this disclosure describes one or more nucleic acid sequence(s) encoding a chimeric receptor; wherein the chimeric receptor comprises: an ECD operatively linked to a second domain; the second domain comprising:
(i)
   (a) a Box 1 and a Box 2 region of G-CSFR;
   (b) at least one signaling molecule binding site of IL-4Rα; or
(ii)
   (a) a Box 1 and a Box 2 region of gp130;
   (b) at least one signaling molecule binding site of gp130; or
(iii)
   (a) a Box 1 and a Box 2 region of Erythropoietin Receptor (EPOR);
   (b) at least one signaling molecule binding site of EPOR; or
(iv)
   (a) a Box 1 and a Box 2 region of G-CSFR;
   (b) at least one signaling molecule binding site of Interferon Alpha and Beta Receptor Subunit 2 (IFNAR2); or
(v)
   (a) a Box 1 and a Box 2 region of Interferon Gamma Receptor 2 (IFNγR2);
   (b) at least one signaling molecule binding site of Interferon Gamma Receptor 1 (IFN γR1).

In certain embodiments of the nucleic acid(s) described herein, the ECD is an ECD of G-CSFR (Granulocyte-Colony Stimulating Factor Receptor). In certain embodiments of the nucleic acid(s) described herein, the TMD is a TMD of G-CSFR and, optionally, the TMD is a wild-type TMD. In certain embodiments of the nucleic acid(s) described herein, the ECD of the G-CSFR is encoded by a nucleic acid sequence set forth in any one of SEQ ID NO. 85, 86 or 87. In certain embodiments, the nucleic acid sequence(s) comprises: (a) a sequence encoding an ICD comprising an amino acid sequence of one or both of SEQ ID NO. 90 or 91; or (b) a sequence encoding an ICD comprising an amino acid sequence of one or both of SEQ ID NO. 90 or 92; or (c) a sequence encoding an ICD comprising an amino acid sequence of SEQ ID NO. 93; or (d) a sequence encoding an ICD comprising an amino acid sequence of SEQ ID NO. 94; or (e) a sequence encoding an ICD comprising an amino acid sequence of one or both of SEQ ID NO. 95 or 96; or (f) a sequence encoding an ICD comprising an amino acid sequence of SEQ ID NO. 97 or 98; or (g) a sequence encoding an ICD comprising an amino acid sequence of SEQ ID NO. 99 or 100.

In certain embodiments, one or more expression vector(s) comprise the nucleic acid sequence(s) described herein. In certain embodiments, the expression vector(s) are selected from the group consisting of: a retroviral vector, a lentiviral vector, an adenoviral vector and a plasmid.

In certain aspects, described herein is a cell comprising a nucleic acid sequence(s) encoding a chimeric receptor described herein. In certain embodiments, the cell is an immune cell, and, optionally the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In certain embodiments, the T cell is selected from the group consisting of a CD8⁺ T cell, cytotoxic CD8⁺ T cell, naïve CD4⁺ T cell, naïve CD8⁺ T cell, helper T cell, regulatory T cell, memory T cell, and γδT cell. In certain embodiments, the cell comprises a nucleic acid sequence(s) described herein. In certain embodiments, the cell comprises an expression vector(s) described herein.

In certain aspects, described herein are methods of selective activation of a chimeric receptor expressed on the surface of a cell, comprising contacting a chimeric receptor with a cytokine that selectively binds the chimeric receptor. In certain embodiments, an activated form of the chimeric receptor forms a homodimer, and, optionally, activation of the chimeric receptor causes a cellular response comprising at least one of proliferation, viability, persistence, cytotoxicity, cytokine secretion, memory, and enhanced activity of a cell expressing the receptor, and, optionally, the chimeric receptor is activated upon contact with the cytokine.

In certain embodiments, the cytokine that selectively binds the chimeric receptor is a G-CSF, and, optionally, the chimeric receptor is activated upon contact with a G-CSF, and, optionally, the G-CSF is a wild-type G-CSF, and, optionally, the extracellular domain of the G-CSFR is a wild-type extracellular domain. In certain embodiments, the chimeric receptor is expressed in a cell, and, optionally, an immune cell, and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell.

In some embodiments, a first population of immune cells expresses the chimeric receptor and a second population of immune cells express a cytokine that binds the chimeric receptor; optionally, wherein one or both of the first and second population(s) of immune cells further express at least one distinct antigen binding signaling receptor(s); and, optionally, wherein the at least one distinct antigen binding signaling receptor(s) comprises at least one CAR. In some embodiments, one or both of the first and second population(s) of immune cells further expresses one or both of: (a) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor. In some embodiments, each of the first and second populations of immune cells express a distinct chimeric receptor comprising a distinct variant ECD of G-CSFR and a distinct variant G-CSF. In some embodiments, the methods further comprise one or more additional populations of immune cells; wherein each additional population of immune cells expresses at least one of (i) a distinct receptor comprising a distinct variant ECD of G-CSFR, (ii) a distinct variant G-CSF, (iii) a distinct an agonistic or antagonistic signaling protein and (iv) a distinct antigen binding signaling receptor.

In certain aspects, described herein are methods of producing a chimeric receptor in a cell, comprising: introducing into the cell one or more nucleic acid sequence(s) described herein or one or more expression vector(s) described herein; and, optionally, the method comprises gene editing; and, optionally, the cell is an immune cell, and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell.

In certain aspects, described herein are methods of treating a subject in need thereof, comprising: administering to the subject a cell expressing a chimeric receptor described herein, and providing to the subject a cytokine that specifically binds the chimeric receptor. In certain embodiments, an activated form of the chimeric receptor forms a homodimer; and, optionally, activation of the chimeric receptor causes a cellular response comprising at least one of proliferation, viability, persistence, cytotoxicity, cytokine secretion, memory, and enhanced activity of a cell expressing the receptor, and, optionally, the chimeric receptor is activated upon contact with the cytokine. In certain embodiments, the cytokine is G-CSF; and, optionally, the G-CSF is a wild-type G-CSF, and, optionally, the extracellular domain of the G-CSFR is a wild-type extracellular domain.

In certain embodiments of the methods described herein, the chimeric receptor is expressed in a cell, and, optionally, an immune cell, and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell.

In certain embodiments, the methods further comprise administering or providing at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s). In certain embodiments, the subject is administered two or more populations of cells each expressing a distinct chimeric receptor comprising a G-CSFR ECD and each expressing a distinct variant form of G-CSF. In certain embodiments, the cells expressing the chimeric receptor further express at least one antigen binding signaling receptor. In certain embodiments, the antigen binding signaling receptor comprises at least one receptor(s) selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In certain embodiments, the antigen binding signaling receptor is a CAR. In certain embodiments, the at least one cytokine(s) or chemokine(s) is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof. In certain embodiments, the cytokine is IL-18. In certain embodiments, the cytokine is human.

In certain embodiments, the method is used to treat cancer such as, but not limited to, bile duct cancer, bladder cancer, breast cancer, cervical cancer, ovarian cancer, colon cancer, endometrial cancer, hematologic malignancies, kidney cancer (renal cell), leukemia, lymphoma, lung cancer, melanoma, non-Hodgkin lymphoma, pancreatic cancer, prostate cancer, sarcoma and thyroid cancer. In certain embodiments, method is used to treat an autoimmune disease. In certain embodiments, the method is used to treat an inflammatory condition. In certain embodiments, the method is used to treat a degenerative disease. In certain embodiments, the method is used to generate natural or engineered cells, tissues or organs for transplantation. In certain embodiments, the method is used to treat or prevent allograft rejection.

In certain embodiments, the method further comprises administering or providing at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s). In some embodiments, the subject is administered two or more populations of cells each expressing a distinct chimeric receptor and each expressing a distinct variant form of a cytokine.

In certain embodiments, the method comprises: i) isolating an immune cell-containing sample; (ii) introducing to the immune cells a nucleic acid sequence encoding the chimeric cytokine receptor; (iii) administering the immune cells from (ii) to the subject; and (iv) contacting the immune cells with the cytokine that binds the chimeric receptor. In certain embodiments, the subject has undergone an immuno-depletion treatment prior to administering or infusing the cells to the subject. In certain embodiments, the immune cell-containing sample is isolated from a subject to whom the cells will be administered. In certain embodiments, the immune cell-containing sample is isolated from a subject distinct from a subject to whom the cells will be administered. In some embodiments, the immune cell-containing sample is generated from cells derived from a subject to whom the cells will be administered, or a subject distinct from a subject to whom the cells will be administered, and, optionally, wherein the cells are stem cells, and, optionally, pluripotent stem cells. In certain embodiments, the immune cells are contacted with the cytokine in vitro prior to administering or infusing the cells to the subject. In certain embodiments, the immune cells are contacted with the cytokine for a sufficient time to activate signaling from the chimeric receptor. In certain embodiments, the cytokine is G-CSF; and, optionally, the G-CSF is a wild-type G-CSF, and, optionally, the extracellular domain of the G-CSFR is a wild-type extracellular domain.

In certain aspects, described herein are kits comprising: at least one expression vector(s) encoding one or more chimeric receptor(s) described herein and instructions for use; and, optionally, the kit comprises at least one cytokine(s) that binds the chimeric receptor(s). In some embodiments, the kit further comprises one or more expression vector(s) encoding at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s). In some embodiments, the kits further comprise one or more expression vector(s) that encode one or more cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof. In some embodiments, the kit further comprises one or more expression vector(s) that encodes at least one antigen binding receptor(s). In some embodiments, the at least one antigen binding receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In some embodiments, the kit further comprises one or more expression vector(s) that encodes one or more CAR(s), and, optionally, wherein the CAR(s) is a mesothelin CAR. In some embodiments, the kit further comprises one or more expression vector(s) encoding one or more distinct chimeric receptor(s) described herein.

In certain aspects, described herein are kit comprising cells encoding one or more chimeric receptor(s) described herein and, optionally, the cells are immune cells; and instructions for use; and, optionally, the kit comprises at least one cytokine(s) that binds the chimeric receptor(s). In some embodiments, the cells further comprise one or more expression vector(s) encoding at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s). In some embodiments, the cells further comprise one or more expression vector(s) that encode at least one cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof. In some embodiments, the cells further comprise one or more expression vector(s) that encode at least one cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof. In some embodiments, the cells further comprise one or more expression vector(s) that encode at least one antigen binding signaling receptor(s). In some embodiments, the at least one antigen binding signaling receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In some embodiments, the cells further comprise one or more expression vector(s) that encode at least one CAR(s), and, optionally, wherein CAR(s) is a mesothelin CAR. In some embodiments, the cells further comprise one or more expression vector(s) encoding at least one distinct chimeric receptor described herein.

In certain aspects, described herein are systems for selective activation of a cell, the system comprising: (i) a receptor comprising a variant extracellular domain (ECD) of Granulocyte Colony-Stimulating Factor Receptor (G-CSFR); and (ii) a variant G-CSF that selectively binds the receptor of (i); and one or both of: (a) at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s) and (b) at least one antigen binding signaling receptor(s). In some embodiments, the at least one additional cytokine(s) or chemokine(s) comprises at least one of interleukin (IL)-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, the receptor NKG2D, and combinations thereof. In some embodiments, the at least one additional cytokine(s) comprises IL-18. In some embodiments, the antigen binding signaling receptor(s) comprises at least one of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In some embodiments, the antigen binding signaling receptor comprises a CAR; and, optionally, the CAR is a mesothelin CAR. In some embodiments, the variant ECD of G-CSFR comprises at least one mutation in a site II interface region, at least one mutation in a site III interface region, or combinations thereof. In some embodiments, the at least one mutation in the site II interface region is located at an amino acid position of the G-CSFR ECD selected from the group consisting of amino acid position 141, 167, 168, 171, 172, 173, 174, 197, 199, 200, 202 and 288 of the sequence shown in SEQ ID NO. 2. In some embodiments, the at least one mutation in the site II interface region of the G-CSFR ECD is selected from the group consisting of R141E, R167D, K168D, K168E, L171E, L172E, Y173K, Q174E, D197K, D197R, M199D, D200K, D200R, V202D, R288D, and R288E of the sequence shown in SEQ ID NO. 2.

In some embodiments, the at least one mutation in the site III interface region of the G-CSFR ECD is selected from the group consisting of amino acid position 30, 41, 73, 75, 79, 86, 87, 88, 89, 91, and 93 of amino acids 2-308 of the sequence shown in SEQ ID NO. 2. In some embodiments, the at least one mutation in the site III interface region of the G-CSFR ECD is selected from the group consisting of S30D, R41E, Q73W, F75KF, S79D, L86D, Q87D, I88E, L89A, Q91D, Q91K, and E93K of the sequence shown in SEQ ID NO. 2. In some embodiments, the G-CSFR ECD comprises a combination of a plurality of mutations of a design number shown in Table 4, 22 and 23; wherein the mutations correspond to an amino acid position of the sequence shown in SEQ ID NO. 2.

In some embodiments, the G-CSFR ECD comprises the mutations: R41E, R141E, and R167D of the sequence shown in SEQ ID NO. 2. In some embodiments, the receptor comprising a variant ECD of G-CSFR is a chimeric receptor.

In some embodiments, the chimeric receptor is operatively linked to at least one second domain; the second domain comprising at least one signaling molecule binding site from an intracellular domain (ICD) of one or more cytokine receptor(s); wherein the at least one signaling molecule binding site is selected from the group consisting of: a STAT3 binding site of G-CSFR, a STAT3 binding site of glycoprotein 130 (gp130), a SHP-2 binding site of gp130, a SHC binding site of IL-2Rβ, a STAT5 binding site of IL-2Rβ, a STAT3 binding site of IL-2Rβ, a STAT1 binding site of IL-2Rβ, a STAT5 binding site of IL-7Rα, a phosphatidylinositol 3-kinase (PI3K) binding site of IL-7Rα, a STAT4 binding site of IL-12Rβ₂, a STAT5 binding site of IL-12Rβ₂, a STAT3 binding site of IL-12Rβ₂, a STAT5 binding site of IL-21R, a STAT3 binding site of IL-21R a STAT1 binding site of IL-21R, an IRS-1 or IRS-2 binding site of IL-4Rα, a STAT6 binding site of IL-4Rα, a SHP-1 or SHP-2 binding site of Erythropoietin Receptor (EPOR), a STAT5 binding site of EPOR, a STAT1 or STAT2 binding site of Interferon Alpha and Beta Receptor Subunit 2 (IFNAR2), and a STAT1 binding site of Interferon Gamma Receptor 1 (IFNγRI), or combinations thereof; optionally, the ICD comprises a Box 1 region and a Box 2 region of a protein selected from the group consisting of G-CSFR, gp130 EPOR, and Interferon Gamma Receptor 2 (IFNγR2), or combinations thereof; and, optionally, the chimeric receptor comprises a third domain comprising a transmembrane domain (TMD) of a protein selected from the group consisting of: G-CSFR, gp130 (Glycoprotein 130), and IL-2Rβ, and, optionally, the TMD is a wild-type TMD.

In some embodiments, the chimeric receptor is operatively linked to at least one second domain; the second domain comprising:
(i)
   (a) a Box 1 and a Box 2 region of gp130; and
   (b) a C-terminal region of IL-2Rβ; or
(ii)
   (a) a Box 1 and a Box 2 region of G-CSFR; and
   (b) a C-terminal region of IL-2Rβ; or
(iii)
   (a) a Box 1 and a Box 2 region of G-CSFR; and
   (b) a C-terminal region of IL-12Rβ₂; or
(iv)
   (a) a Box 1 and a Box 2 region of G-CSFR; and
   (b) a C-terminal region of IL-21R; or
(v)
   (a) a Box 1 and a Box 2 region of IL-2Rβ; and
   (b) a C-terminal region of IL-2Rβ; or
(vi)
   (a) a Box 1 and a Box 2 region of G-CSFR; and
   (b) a C-terminal region of IL-7Rα; or
(vii)
   (a) a Box 1 and a Box 2 region of G-CSFR;
   (b) a C-terminal region of IL-4Rα; or
(viii)
   (a) a Box 1 and a Box 2 region of gp130;
   (b) a C-terminal region of gp130; or
(ix)
   (a) a Box 1 and a Box 2 region of Erythropoietin Receptor (EPOR);
   (b) a C-terminal region of EPOR; or
(x)
   (a) a Box 1 and a Box 2 region of G-CSFR;
   (b) a C-terminal region of Interferon Alpha and Beta Receptor Subunit 2 (IFNAR2); or
(xi)
   (a) a Box 1 and a Box 2 region of Interferon Gamma Receptor 2 (IFNγR2);
   (b) a C-terminal region of Interferon Gamma Receptor 1 (IFN γR1).

In some embodiments, the ECD is N-terminal to the TMD, and TMD N-terminal to the ICD. In some embodiments, the receptor comprising a variant ECD of G-CSFR is expressed on the cell. In some embodiments, the cell is an immune cell and, optionally the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell is selected from the group consisting of a CD8⁺ T cell, cytotoxic CD8⁺ T cell, naïve CD4⁺ T cell, naïve CD8⁺ T cell, helper T cell, regulatory T cell, memory T cell, and γδT cell. In some embodiments, activation of the receptor comprising a variant ECD of G-CSFR by the variant G-CSF causes a cellular response comprising at least one of proliferation, viability, persistence, cytotoxicity, cytokine secretion, memory, enhanced activity of a cell expressing the receptor, and combinations thereof. In some embodiments, the variant G-CSF comprises at least one mutation in a site II interface region, at least one mutation in a site III interface region, or combinations thereof. In some embodiments, the at least one mutation in the site II interface region of the variant G-CSF is located at an amino acid position selected from the group consisting of amino acid position 12, 16, 19, 20, 104, 108, 109, 112, 115, 116, 118, 119, 122 and 123 of the sequence shown in SEQ ID NO. 1. In some embodiments, the at least one mutation in the site II interface region of the variant G-CSF is selected from a group of mutations selected from the group consisting of: S12E, S12K, S12R, K16D, L18F, E19K, Q20E, D104K, D104R, L108K, L108R, D109R, D112R, D112K, T115E, T115K, T116D, Q119E, Q119R, E122K, E122R, and E123R. In some embodiments, the at least one mutation in the site III interface region of the variant G-CSF is selected from a group of mutations selected from the group consisting of: 38, 39, 40, 41, 46, 47, 48, 49, and 147 of the sequence shown in SEQ ID NO. 1.

In some embodiments, the at least one mutation in the site III interface region of the variant G-CSF is selected from a group of mutations selected from the group consisting of: T38R, Y39E, K40D, K40F, L41D, L41E, L41K, E46R, L47D, V48K, V48R, L49K, and R147E. In some embodiments, the cell expresses both the receptor comprising the variant ECD of G-CSFR and the at least one additional cytokine(s) or chemokine(s). In some embodiments, two or more populations of cells each express a one or more distinct chimeric receptor(s) comprising a G-CSFR ECD and each express one or more distinct variant forms of G-CSF. In some embodiments, the first population of immune cells further expresses one or both of: (a) at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor(s). In some embodiments, the cell is an immune cell; and wherein the immune cell further expresses the antigen binding signaling receptor; and wherein the antigen binding signaling receptor selectively binds to an antigen expressed on a second cell. In some embodiments, the antigen binding signaling receptor(s) comprises a chimeric antigen receptor (CAR), and, optionally, a mesothelin CAR. In some embodiments, the additional cytokine comprises IL-18. In some embodiments, the second cell is a cancer cell.

In certain aspects, the present disclosure describes one or more nucleic acid sequence(s) encoding a system described herein. In certain aspect, the present disclosure describes one or more expression vector(s) comprising a nucleic acid sequence(s) described herein. In certain aspects, the present disclosure describes one or more cell(s) engineered to express a system described herein. In some embodiments, the cell(s) is an immune cell and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T-cell is selected from the group consisting of a CD8⁺ T cell, cytotoxic CD8⁺ T cell, naïve CD8⁺ T cell, naïve CD4⁺ T cell, helper T cell, regulatory T cell, memory T cell, and γδT cell.

In certain aspects, described herein are methods of selective activation of a receptor comprising a variant ECD of G-CSFR expressed on the surface of a cell, comprising: introducing into the cell one or more nucleic acid sequence(s) encoding one or more receptor(s) comprising a variant ECD of G-CSFR of a system described herein; and one or both of (i)) at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s) of a system described herein; and (ii) at least one antigen binding signaling receptor(s) of a system described herein; and contacting the receptor(s) comprising the variant ECD of G-CSFR with one or more variant G-CSF or the variant G-CSF of a system described herein. In some embodiments, the receptor(s) is expressed on an immune cell, and, optionally the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell is selected from the group consisting of a CD8⁺ T cell, cytotoxic CD8⁺ T cell, naïve CD4⁺ T cell, naïve CD8⁺ T cell, helper T cell, regulatory T cell, memory T cell, and γδT cell. In some embodiments, the selective activation of the receptor(s) expressed on the immune cell causes a cellular response comprising at least one of proliferation, viability, persistence, cytotoxicity, cytokine secretion, memory, enhanced activity of the immune cell, and combinations thereof. In some embodiments, a first population of immune cells expresses the receptor comprising the variant ECD of G-CSFR and a second population of immune cells express the variant G-CSF; optionally, wherein one or both of the first and second population(s) of immune cells further express at least one distinct antigen binding signaling receptor(s); and, optionally, wherein the at least one distinct antigen binding signaling receptor(s) comprises at least one CAR. In some embodiments, one or both of the first and second population(s) of immune cells further expresses one or both of: (a) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor(s).

In some embodiments, each of the first and second populations of immune cells express at least one distinct receptor(s) comprising a distinct variant ECD of G-CSFR and at least one distinct variant G-CSF. In some embodiments, the method further comprises one or more additional populations of immune cells; wherein each additional population of immune cells expresses at least one of (i) a distinct receptor comprising a distinct variant ECD of G-CSFR, (ii) a distinct variant G-CSF, (iii) a distinct an agonistic or antagonistic signaling protein and (iv) a distinct antigen binding signaling receptor.

In certain aspects, the present disclosure describes methods of producing a cell expressing one or more receptor(s) comprising a variant ECD of G-CSFR of a system described herein; and one or both of: (i)) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s) of a system described herein; and (ii) at least one antigen binding signaling receptor of the system of a system described herein; the method comprising introducing to the cells one or more nucleic acid(s) or expression vector(s) encoding the receptor, and one or both of (i), and (ii). In some embodiments, a first population of immune cells expresses the receptor comprising the variant ECD of G-CSFR and a second population of immune cells express the variant G-CSF. In some embodiments, or both of the first and second population(s) of immune cells further expresses one or both of: (a) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor(s).

In certain aspects, described herein are methods of increasing an immune response in a subject in need thereof, comprising administering to the subject the immune cell(s) described herein. In certain aspects, described herein are methods of treating a disease in a subject in need thereof, comprising: administering to the subject the immune cell(s) herein. In some embodiments, the methods further comprise administering or providing a variant G-CSF to the subject. In some embodiments, the method is used to treat cancer. In some embodiments, the method is used to treat an inflammatory condition. In some embodiments, the method is used to treat an autoimmune disease or condition. In some embodiments, the method is used to treat a degenerative disease. In some embodiments, the method is used to generate natural or engineered cells, tissues or organs for transplantation. In some embodiments, the method is used to prevent or treat graft rejection. In some embodiments, the method is used to treat an infectious disease. In some embodiments, the method further comprises administering or providing at least one additional active agent; optionally wherein the at least one additional active agent comprises at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s).

In certain aspects, described herein are methods of treating a subject in need thereof, wherein the method comprises: (i) isolating an immune cell-containing sample; (ii) introducing the immune cells with one or more nucleic acid sequence(s) encoding one or more receptor(s) comprising the variant ECD of G-CSFR of the system described herein; and one or both of: (a) at least one additional active agent; optionally wherein the at least one additional active agent comprises at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s) of a system described herein; and optionally, (b) the antigen binding signaling receptor of a system described herein; (iii) administering the immune cell(s) from (ii) to the subject; and (iv) contacting the immune cell(s) with a variant G-CSF that specifically binds the receptor(s) comprising the variant ECD of G-CSFR. In some embodiments, the subject has undergone an immuno-depletion treatment prior to administering or infusing the immune cell(s) to the subject. In some embodiments, the immune cell-containing sample is isolated from the subject to whom the cell(s) are administered. In some embodiments, the immune cell-containing sample is isolated from a subject distinct from the subject to whom the cell(s) are administered. In some embodiments, the immune cell-containing sample is generated from source cells derived from a subject to whom the source cells will be administered, and, optionally, wherein the cells are stem cells, and, optionally, pluripotent stem cells. In some embodiments, the immune cell-containing sample is generated from source cells derived from a subject distinct from a subject to whom the source cells will be administered, and, optionally, wherein the cells are stem cells, and, optionally, pluripotent stem cells. In some embodiments, the immune cell(s) are contacted with the variant G-CSF or additional cytokine(s) or chemokine(s) in vitro prior to administering the immune cell(s) to the subject. In some embodiments, the immune cell(s) are contacted with the variant G-CSF for a sufficient time to activate signaling from the receptor(s) comprising the variant ECD of G-CSFR of a system described herein.

In certain aspects, described herein are kits comprising cells encoding: one or more receptor(s) comprising the variant ECD of G-CSFR of a system described herein; and one or both of: (a) one or more additional cytokine(s) and chemokine(s) of a system described herein; and, (b) one or more antigen binding signaling receptor(s) of a system described herein; and instructions for use; and optionally, wherein the cells are immune cells. In certain aspects, described herein are kits comprising: (i) one or more nucleic acid sequence(s) or expression vector(s) encoding the receptor(s) comprising the variant ECD of G-CSFR of a system described herein; and one or both of: (a) at least one additional active agent(s); optionally wherein the at least one additional active agent(s) comprises at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s) of a system described herein; and, (b) one or more antigen binding signaling receptors of a system described herein; and (ii) one or more variant G-CSF; and (iii) instructions for use; wherein the receptor and one or both of (a) and (b) are located on the same or separate nucleic acid sequence or expression vector.

In certain aspects, described herein are kits comprising: (i) cells comprising one or more nucleic acid sequence(s) or expression vector(s) encoding the receptor(s) comprising variant ECD of G-CSFR of a system described herein; and one or both of: (a) at least one additional active agent(s); optionally wherein the at least one additional active agent(s) comprises at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s) of a system described herein; and (b) at least one antigen binding signaling receptor(s) of a system described herein; and (ii) instructions for use; and, optionally, wherein the kit comprises one or more variant G-CSF that specifically binds the receptor(s) comprising the variant ECD of G-CSFR.

In certain aspects, disclosed herein are chimeric receptors, comprising: (i) an extracellular domain (ECD) of Interleukin Receptor alpha (IL-7Rα); (ii) a transmembrane domain (TMD); and (iii) an intracellular domain (ICD) of a cytokine receptor that is distinct from a wild-type, human IL-7Rα intracellular signaling domain set forth in SEQ ID NO:109; wherein the ECD and TMD are each operatively linked to the ICD. In some embodiments, the carboxy terminus (C-terminus) of the ECD is linked to the amino terminus (N-terminus) of the TMD, and the C-terminus of TMD is linked to the N-terminus of the ICD. In some embodiments, the ECD is the ECD of native human IL-7Rα. In some embodiments, the TMD is the TMD of IL-7Rα. In some embodiments, the TMD is the TMD of native human IL-7Rα. In some embodiments, the ICD comprises at least one signaling molecule binding site from an intracellular domain of a cytokine receptor, and, optionally, the at least one signaling molecule binding site comprises: (a) a JAK1 binding site (Box 1 and 2 region) of IL-2Rβ, IL-4Rα, IL-7Rα, IL-21R, or gp130; (b) a SHC binding site of IL-2Rβ; (c) a STAT5 binding site of IL-2Rβ or IL-7Rα; (d) a STAT3 binding site of IL-21R or gp130; (e) a STAT4 binding site of IL-12Rβ2; (f) a STAT6 binding site of IL-4Rα; (g) an IRS-1 or IRS-2 binding site of IL-4Rα; and (h) a SHP-2 binding site of gp130; (i) a PI3K binding site of IL-7Rα; or combinations thereof. In some embodiments, the ICD comprises at least an intracellular signaling domain of a receptor that is activated homodimerization or by heterodimerization with the common gamma chain (gc). In some embodiments, the ICD comprises at least an intracellular signaling domain of a cytokine receptor selected from the group consisting of: IL-2Rβ (Interleukin-2 receptor beta), IL-4Rα (Interleukin-4 Receptor alpha), IL-9Rα (Interleukin-9 Receptor alpha), IL-12Rβ2 (Interleukin-12 Receptor), IL-21R (Interleukin-21 Receptor) and glycoprotein 130 (gp130), and combinations thereof.

In certain aspects, described herein are chimeric receptors, comprising an ECD of IL-7Rα and a TMD operatively linked to an ICD, the ICD comprising:
(i)
   (a) a Box 1 and a Box 2 region of IL-2Rβ;
   (b) a SHC binding site of IL-2Rβ; and
   (c) a STAT5 binding site of IL-2Rβ; or
(ii)
   (a) a Box 1 and a Box 2 region of IL-7Rα;
   (b) a SHC binding site of IL-2Rβ; and
   (c) a STAT5 binding site of IL-2Rβ; or
(iii)
   (a) a Box 1 and a Box 2 region of IL-2Rβ;
   (b) a SHC binding site of IL-2Rβ;
   (c) a STAT5 binding site of IL-2Rβ; and
   (d) a STAT4 binding site of IL-12Rβ2; or
(iv)
   (a) a Box 1 and a Box 2 region of IL-7Rα;
   (b) a SHC binding site of IL-2Rβ;
   (c) a STAT5 binding site of IL-2Rβ; and
   (d) a STAT4 binding site of IL-12Rβ2; or
(v)
   (a) a Box 1 and a Box 2 region of IL-21R; and
   (b) a STAT3 binding site of IL-21R; or
(vi)
   (a) a Box 1 and a Box 2 region of IL-7Rα; and
   (b) a STAT3 binding site of IL-21R; or
(vii)
   (a) a Box 1 and a Box 2 region of IL-21R;
   (b) a STAT3 binding site of IL-21R; and
   (c) a STAT5 and PI3 kinase binding site of IL-7Rα;
(viii)
   (a) a Box 1 and a Box 2 region of IL-7Rα;
   (b) a STAT3 binding site of IL-21R; and
   (c) a STAT5 and PI3 kinase binding site of IL-7Rα;
(ix)
   (a) a Box 1 and a Box 2 region of IL-2Rβ;
   (b) a SHC binding site of IL-2Rβ;
   (c) a STAT5 binding site of IL-2Rβ; and
   (d) a STAT3 binding site of IL-21R; or
(x)
   (a) a Box 1 and a Box 2 region of IL-7Rα;
   (b) a SHC binding site of IL-2Rβ;
   (c) a STAT5 binding site of IL-2Rβ; and
   (d) a STAT3 binding site of IL-21R; or
(xi)
   (a) a Box 1 and a Box 2 region of IL-2Rβ;
   (b) a SHC binding site of IL-2Rβ;
   (c) a STAT5 binding site of IL-2Rβ;
   (d) a STAT4 binding site of IL12Rβ2; and
   (e) a STAT3 binding site of IL-21R; or
(xii)
   (a) a Box 1 and a Box 2 region of IL-7Rα;
   (b) a SHC binding site of IL-2Rβ;
   (c) a STAT5 binding site of IL-2Rβ;
   (d) a STAT4 binding site of IL12Rβ2; and
   (e) a STAT3 binding site of IL-21R; or
(xiii)
   (a) a Box 1 and a Box 2 region of IL-4Rα;
   (b) an IRS-1 or IRS-2 binding site of IL-4Rα; and
   (c) a STAT6 binding site of IL-4Rα; or
(xiv)
   (a) a Box 1 and a Box 2 region of IL-7Rα;
   (b) an IRS-1 or IRS-2 binding site of IL-4α; and
   (c) a STAT6 binding site of IL-4α; or
(xv)
   (a) a Box 1 and a Box 2 region of gp130;
   (b) a SHP-2 binding site of gp130; and
   (c) a STAT3 binding site of gp130; or
(xvi)
   (a) a Box 1 and a Box 2 region of IL-7Rα;
   (b) a SHP-2 binding site of gp130; and
   (c) a STAT3 binding site of gp130.
      In some embodiments, (b) is N-terminal to (c); or (c) is N-terminal to (b); or (c) is N-terminal to (d); or (d) is N-terminal to (c); or (d) is N-terminal to (e); or (e) is N-terminal to (d).

In some embodiments, the ICD comprises a sequence at least 80%, at least 90%, at least 91%, at least 92%, at least 93%, at least 94%, at least 95%, at least 96%, at least 97%, at least 98%, at least 99% or 100% identical to a sequence shown in at least one of SEQ ID NO: 192-214.

In some embodiments, an activated form of the chimeric receptor forms a heterodimer and, optionally, the activation of the chimeric receptor causes a cellular response comprising at least one of proliferation, viability, persistence, cytotoxicity, cytokine secretion, memory, and enhanced activity of a cell expressing the chimeric receptor, and, optionally, the chimeric receptor is activated upon contact with interleukin (IL)-7. In some embodiments, the IL-7 is a wild-type, human IL-7. In some embodiments, the IL-7 harbors 1, 2, 3, 4 or 5 mutations compared to wild-type IL-7. In some embodiments, the IL-7 comprises one or more chemical modifications.

In some embodiments, the chimeric receptor is expressed on a cell. the cell is an immune cell and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell, is selected from the group consisting of a CD8⁺ T cell, cytotoxic CD8⁺ T cell, naïve CD4⁺ T cell, naïve CD8⁺ T cell, helper T cell, regulatory T cell, memory T cell, and γδT cell. In some embodiments, activation of the receptor by IL-7 causes a cellular response comprising at least one of proliferation, viability, persistence, cytotoxicity, cytokine secretion, memory, and enhanced activity of a cell expressing the receptor.

In certain aspects, the present disclosure describes one or more nucleic acid sequence(s) encoding a receptor described herein. In certain aspects, the present disclosure describes one or more expression vector(s) comprising a nucleic acid sequence(s) described herein. In certain aspects, the present disclosure describes a cell comprising the nucleic acid sequence(s), or the expression vector(s) described herein. In some embodiments, the cell is an immune cell and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell is selected from the group consisting of CD8⁺ T cells, cytotoxic CD8⁺ T cells, naïve CD4⁺ T cells, naïve CD8⁺ T cells, helper T cells, regulatory T cells, memory T cells, and γδT cells.

In certain aspects, the present disclosure describes a system for activation of a receptor expressed on a cell surface, the system comprising: (a) a chimeric receptor described herein; and (b) IL-7.

In certain aspects, the present disclosure describes a system for activation of an immune cell, the system comprising: (a) a chimeric receptor described herein; (b) IL-7; and (c) an antigen binding signaling receptor.

In certain aspects, the present disclosure describes a system for activation of an immune cell, the system comprising: (a) a chimeric receptor described herein; (b) IL-7; and (c) at least one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent.

In some embodiments, the system further comprises at least one antigen binding signaling receptor. In some embodiments, the at least one antigen binding signaling receptor comprises at least one receptor selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In some embodiments, the at least one antigen binding signaling receptor is a CAR. In some embodiments, the cytokine or chemokine is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, the receptor NKG2D, and combinations thereof. In some embodiments, the cytokine is IL-18. In some embodiments, the cytokine is human.

In certain aspects, described herein are methods of activation of a chimeric receptor expressed on the surface of a cell, comprising: contacting the chimeric receptor with IL-7 to activate the chimeric receptor; wherein the chimeric receptor comprises: (i) an extracellular domain (ECD) of IL-7Rα; (ii) a transmembrane domain (TMD); and (iii) an intracellular domain (ICD) of a cytokine receptor that is distinct from the wild-type, human IL-7Rα intracellular signaling domain set forth in SEQ ID NO:109; wherein the ECD and TMD are each operatively linked to the ICD. In some embodiments, the chimeric receptor is a chimeric receptor described herein.

In certain aspects, described herein is a method of producing a chimeric receptor in a cell, the method comprising: introducing into the cell a nucleic acid sequence(s) described herein or the expression vector(s) described herein. In some embodiments, the method further comprises editing the sequence(s) or sequence(s) of the vector(s) into the genome of the cell.

In some embodiments of the methods described herein, the cell is an immune cell; and, optionally, the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell is selected from the group consisting of CD8⁺ T cells, cytotoxic CD8⁺ T cells, naïve CD4⁺ T cells, naïve CD8⁺ T cells, helper T cells, regulatory T cells, memory T cells, and γδT cells.

In certain aspects, described herein is a method of increasing an immune response in a subject in need thereof, comprising: administering to the subject cell(s) expressing a chimeric receptor described herein, and administering or providing IL-7 to the subject.

In certain aspects, described herein is a method of treating a subject in need thereof, comprising: administering to the subject cell(s) expressing a chimeric receptor described herein, and administering or providing IL-7 to the subject. In some embodiments, the method is used to treat cancer. In some embodiments, the method is used to treat an autoimmune disease. In some embodiments, the method is used to treat an inflammatory condition. In some embodiments, the method is used to treat a degenerative disease. In some embodiments, the method is used to generate natural or engineered cells, tissues or organs for transplantation. In some embodiments, the method is used to prevent or treat graft rejection. In some embodiments, the method is used to treat an infectious disease.

In certain embodiments, the method is used to treat a degenerative disease or condition. Examples of degenerative diseases or conditions include, but are not limited to, neurodegenerative diseases and conditions related to aging.

In certain embodiments, the method is used to generate natural or engineered cells, tissues or organs for transplantation.

In some embodiments, the methods further comprise administering or providing at least one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the subject is administered cells expressing at least one additional distinct chimeric receptor. In some embodiments, the at least one additional distinct chimeric receptor is a chimeric receptor comprising a variant ECD of Granulocyte Cell Stimulating Factor Receptor (G-CSFR). In some embodiments, the cells expressing the at least one additional distinct chimeric receptor comprising a variant ECD of G-CSFR are contacted with one or more variant G-CSF, and optionally, the subject is administered one or more variant G-CSF. In some embodiments, the method comprises: i) isolating an immune cell-containing sample; (ii) transducing or transfecting the immune cell(s) with nucleic acid sequence(s) encoding the chimeric cytokine receptor(s); (iii) administering the immune cell(s) from (ii) to the subject; and (iv) contacting the immune cells with IL-7.

In some embodiments, the methods further comprise introducing to the immune cell(s) with nucleic acid sequence(s) encoding at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the at least one cytokine or chemokine is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, the receptor NKG2D, and combinations thereof. In some embodiments, the methods further comprise introducing to the immune cell(s) with nucleic acid sequence(s) encoding at least one antigen binding signaling receptor. In some embodiments, the at least one antigen binding signaling receptor is selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In some embodiments, the subject has undergone an immuno-depletion treatment prior to administering the cells to the subject. In some embodiments, the immune cell-containing sample is isolated from the subject to whom the cells are administered. In some embodiments, the immune cell-containing sample is isolated from a subject distinct from a subject to whom the cells will be administered. In some embodiments, the immune cell-containing sample is generated from cells derived from a subject distinct from a subject to whom the cells will be administered, and, optionally, wherein the cells are stem cells, and, optionally, pluripotent stem cells. In some embodiments, the immune cells are contacted with one or both of IL-7 or a variant G-CSF in vitro prior to administering the cells to the subject. In some embodiments, the immune cells are contacted with one or both of IL-7 or a variant G-CSF for a sufficient time to activate signaling from a chimeric receptor described herein.

In some embodiments, the cells administered to the subject further express at least one antigen binding signaling receptor selected from the group consisting of: a native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In some embodiments, the cells administered to the subject further express a receptor comprising a variant ECD of G-CSFR. In some embodiments, the variant ECD of G-CSFR comprises at least one mutation in a site II interface region, at least one mutation in a site III interface region, or combinations thereof. In some embodiments, the cells administered to the subject further express IL-18.

In certain aspects, described herein are kits, comprising: cells encoding a chimeric receptor described herein, and, optionally, the cells are immune cells; and instructions for use; and, optionally, the kit comprises IL-7 and, optionally, the kit comprises a variant G-CSF described herein. In certain aspects, described herein are kits, comprising: one or more expression vector(s) comprising the nucleic acid sequence(s) encoding a chimeric receptor described herein and instructions for use; and, optionally, the kit comprises IL-7 and, optionally, the kit comprises a variant G-CSF described herein. In some embodiments, the kit further comprises one or more expression vector(s) that encode at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the kit further comprises one or more expression vector(s) that encode a cytokine or chemokine selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, the receptor NKG2D, and combinations thereof. In some embodiments, the kit further comprises one or more expression vector(s) that encodes at least one receptor selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In some embodiments, the kit further comprises an expression vector that encodes a chimeric antigen receptor. In some embodiments, the cells further comprise one or more expression vector(s) that encode at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the cells further comprise one or more expression vector(s) that encode at least one cytokine or chemokine selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, the receptor NKG2D, and combinations thereof. In some embodiments, the cells further comprise one or more expression vector(s) that encode at least one antigen binding signaling receptor(s). In some embodiments, the at least one antigen binding signaling receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor and combinations thereof. In some embodiments, the cells further comprise one or more expression vector(s) that encode at least one CAR(s).

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 presents a diagram showing the structures of the Site II and III interfaces of the 2:2 G-CSF:G-CSFR heterodimeric complex.

FIG. 2 presents a diagram outlining the strategy used for the G-CSF:G-CSFR interface design.

FIG. 3 presents a graph showing the energetic components of site II interface interactions.

FIG. 4 presents a diagram showing the structure of the site II interface and the interactions of Arg167 (left) and Arg141 (right) of G-CSFR(CRH) with G-CSF residues at site II interface.

FIG. 5 presents a diagram showing wild type G-CSF at site II (left) and overlay of the same region of a triplicate ZymeCAD™ mean-field pack of site II design #35 (right).

FIG. 6 presents images of SDS-PAGE showing results of G-CSF pulldown assays of G-CSF designs #: 6, 7, 8, 9, 15, 17, 30, 34, 35, and 36 (top panel) and co-expressed and purified site II design complexes #: 6, 7, 8, 9, 15, 17, 30, 34, 35, and 36 (bottom panel), first lane WT G-CSF control, second lane WT G-CSF:G-CSFR(CRH) control.

FIG. 7 presents images of SDS-PAGE showing results of G-CSF pulldown assays of G-CSF designs #: 6, 7, 8, 9, 15, 17, 30, 34, 35, and 36 co-expressed with WT-G-CSFR (top panel) and G-CSF pulldown assays of WT G-CSF co-expressed with G-CSFR designs #: 6, 7, 8, 9, 15, 17, 30, 34, 35, and 36 (bottom panel, first lane WT:WT G-CSF:G-CSFR pulldown control.

FIG. 8 presents a graph showing the energetic components of site III interface interactions.

FIG. 9 presents a diagram showing the structure of the site III interface and interactions of R41 (left) and E93 (right) of G-CSFR(Ig) with G-CSF residues at site III interface.

FIG. 10 presents images of SDS-PAGE showing results of G-CSF pulldown assays of designs #401 and 402 (top panel) and co-expressed and purified site II/III design complexes #401 and 402, and with WT G-CSFR and pulldown of WT G-CSF co-expressed with design #401 and 402 G-CSFR (bottom panel), first lane WT G-CSF control, second lane WT G-CSF:G-CSFR(Ig-CRH) control.

FIG. 11 presents graphs showing size-exclusion chromatography profile of WT (SX75) G-CSF and design 130 (SX75), 303 (SX200) and 401 (SX200) G-CSF_{E} mutants post TEV cleavage.

FIG. 12 presents graphs showing size-exclusion chromatography profile of purified WT (SX75) and purified design 401 (SX200) and 402 (SX200) G-CSFR(Ig-CRH)_{E} mutants post TEV cleavage.

FIG. 13 presents graphs showing binding SPR sensorgrams for WT, designs #130, #401, #402 G-CSF, binding to their cognate or mispaired G-CSFR(Ig-CRH). Each G-CSF vs G-CSFR pair is labelled in the bottom of each panel. Representative steady state fit used to derive KDs for the cognate pair of design #401 and #402 are shown under their respective sensorgrams.

FIG. 14 presents graphs showing: Top left panel: DSC thermograms of WT G-CSF, design #130 and #134 G-CSF_{E}; Top right panel: DSC thermograms of WT G-CSFR(Ig-CRH), design #130 and #134 G-CSFR(Ig-CRH)_{E}; Bottom left panel: DSC thermograms of design #401 and #402 G-CSF_{E}; Bottom right: DSC thermograms of design #300, #303, #304 and #307 G-CSF_{E}.

FIG. 15 presents graphs showing results of bromo-deoxyuridine (BrdU) assays showing proliferation 32D-IL-2RβIL2Rb cells expressing A) G-CSFR_{WT}-ICD_{IL-2Rb} (homodimer) or B) G-CSFR_{WT}-ICD_{IL-2Rb}+G-CSFR_{WT}-ICD_{gc} (heterodimer). Cells were stimulated with no cytokine, IL-2 (300 IU/ml) or G-CSF_{WT} (100 ng/ml in A or 30 ng/ml in B).

FIG. 16 presents graphs showing results of BrdU assays showing proliferation of 32D-IL-2Rβ cells expressing A) G-CSFR₁₃₇-ICD_{gp130-IL-2Rb} (homodimer); or B) G-CSFR₁₃₇-ICD_{IL-2Rb}+G-CSFR₁₃₇-ICD_{gc} (heterodimer). Cells were stimulated with no cytokine, IL-2 (300 IU/ml), G-CSF_{WT} (30 ng/ml), or G-CSFR₁₃₇ (30 ng/ml).

FIG. 17 presents graphs showing results of BrdU assays showing proliferation of 32D-IL-Rβ cells expressing: A) G-CSFR_{WT}-ICD_{gp130-IL-2Rb} (homodimer); or B) G-CSFR_{WT}-ICD_{IL-2Rb}+G-CSFR_{WT}-ICD_{gc} (heterodimer). Cells were stimulated with no cytokine, IL-2 (300 IU/ml), G-CSF_{WT} (30 ng/ml), or G-CSFR₁₃₇ (30 ng/ml).

FIG. 18 presents western blots showing signaling of 32D-IL-2Rβ cells expressing: G-CSFR_{WT}-ICD_{gp130-IL-2Rb} (homodimer), G-CSFR₁₃₇-ICD_{gp130-IL-2Rb} (homodimer), G-CSFR_{WT}-ICD_{IL-2Rb}+G-CSFR_{WT}-ICD_{gc} (heterodimer) or G-CSFR₁₃₇-ICD_{IL-2Rb}+G-CSFR₁₃₇-ICD_{gc} (heterodimer). Cells were stimulated with no cytokine, IL-2 (300 IU/ml), G-CSF_{WT} (30 ng/ml), or G-CSFR₁₃₇ (30 ng/ml).

FIG. 19 presents graphs showing the results of BrdU incorporation assays to assess cell cycle progression of primary murine T cells expressing the indicated chimeric receptors (or non-transduced cells) in response to stimulation with no cytokine, IL-2 or WT, 130, 304 or 307 cytokine. A and B represent experimental replicates.

FIG. 20 presents a schematic of native IL-2Rβ, IL-2Rγc, and G-CSFR subunits, as well as the G2R-1 receptor subunit designs.

FIG. 21 presents graphs showing the expansion (fold change in cell number) of 32D-IL-2Rβ cells (which is the 32D cell line stably expressing the human IL-2Rβ subunit) expressing the indicated G-CSFR chimeric receptor subunits and stimulated with WT G-CSF, IL-2 or no cytokine. G/γc was tagged at its N-terminus with a Myc epitope (Myc/G/γc), and G/IL-2Rβ was tagged at its N-terminus with a Flag epitope (Flag/G/IL-2Rβ); these epitope tags aid detection by flow cytometry and do not impact the function of the receptors. In addition, the lower panels in B-D show the percentage of cells expressing the G-CSFR ECD (% G-CSFR+) under each of the culture conditions. Squares represent cells stimulated with IL-2. Triangles represent cells stimulated with G-CSF. Circles represent cells not stimulated with a cytokine.

FIG. 22 presents graphs showing the expansion (fold change in cell number) of human T cells expressing the Flag-tagged G/IL-2Rβ subunit alone, the Myc-tagged G/γc subunit alone, or the full-length G-CSFR. A-D) PBMC-derived T cells; E-H) tumor-associated lymphocytes (TAL). Squares represent cells stimulated with IL-2. Triangles represent cells stimulated with G-CSF. Circles represent cells not stimulated with a cytokine.

FIG. 23 presents a schematic of native and chimeric receptors, showing JAK, STAT, Shc, SHP-2 and PI3K binding sites. The shading scheme includes receptors from FIG. 20.

FIG. 24 presents a schematic of chimeric receptors, showing JAK, STAT, Shc, SHP-2 and PI3K binding sites. The shading scheme includes receptors from FIGS. 20 and 23.

FIG. 25 presents a diagram of the lentiviral plasmid containing the G2R-2 cDNA insert.

FIG. 26 presents graphs showing G-CSFR ECD expression assessed by flow cytometry in cells transduced with G2R-2. A) 32D-IL-2Rβ cell line; B) PBMC-derived human T cells and human tumor-associated lymphocytes (TAL).

FIG. 27 presents graphs showing the expansion (fold change in cell number) of cells expressing G2R-2 compared to non-transduced cells. A) Human PBMC-derived T cells; B, C) Human tumor-associated lymphocytes (TAL) from two independent experiments. Squares represent cells stimulated with IL-2. Triangles represent cells stimulated with G-CSF. Circles represent cells not stimulated with cytokine.

FIG. 28 presents graphs showing expansion (fold change in cell number) of CD4- or CD8-selected human tumor-associated lymphocytes expressing G2R-2 compared to non-transduced cells. A) Non-transduced CD4-selected cells; B) Non-transduced CD8-selected cells; C) CD4-selected cells transduced with G2R-2; D) CD8-selected cells transduced with G2R-2. Dotted gray line represents cells stimulated with IL-2. Solid black line represents cells stimulated with G-CSF. Dashed gray line represents cells not stimulated with a cytokine.

FIG. 29 presents graphs showing expansion (fold change in cell number) of CD4+ or CD8+ tumor-associated lymphocytes expressing G2R-2. The cells were initially expanded in G-CSF or IL-2, as indicated. Cells were then plated in either IL-2, G-CSF or medium only. Solid gray line represents cells stimulated with IL-2. Solid gray line represents cells stimulated with IL-2. Solid black line represents cells stimulated with G-CSF. Dashed light gray line represents cells expanded in IL-2 and then stimulated with medium only. Dashed dark gray line represents cells expanded in G-CSF and then stimulated with medium only.

FIG. 30 presents a graph showing immunophenotype (by flow cytometry) of CD4- or CD8-selected tumor-associated lymphocytes (TAL) expressing G2R-2 chimeric receptor construct versus non-transduced cells, after expansion in G-CSF or IL-2. A) Percentage of live cells showing a CD4+, CD8+ or CD3−CD56+ cell surface phenotype. B) Percentage of live cells showing the indicated cell surface phenotypes based on CD45RA and CCR7 expression.

FIG. 31 presents graphs showing the results of BrdU incorporation assays to assess proliferation of primary human T cells expressing G2R-2 versus non-transduced cells. T cells were selected by culture in IL-2 or G-CSF, as indicated, prior to the assay. A) Tumor-associated lymphocytes; B) PBMC-derived T cells.

FIG. 32 presents graphs showing the results of BrdU incorporation assays to assess proliferation of primary murine T cells expressing G2R-2 or the single-chain G/IL-2Rβ (a component of G2R-1) versus mock-transduced cells. A) Transduction efficiency as reflected by the percentage of cells expressing the G-CSFR ECD (by flow cytometry) after culture in the indicated cytokines; B) Percent BrdU incorporation in all live cells in response to the indicated cytokines; C) Percent BrdU incorporation by cells expressing the G-CSFR ECD (G-CSFR+ cells). All cells were expanded in IL-2 for 3 days prior to assay. Squares represent cells stimulated with IL-2. Triangles represent cells stimulated with G-CSF. Circles represent cells not stimulated with a cytokine.

FIG. 33 presents western blots to detect the indicated cytokine signaling events in human primary T cells expressing G2R-2 versus non-transduced cells. β-actin, total Akt and histone H3 serve as a protein loading controls. A, B) Tumor-associated lymphocytes (TALs); C) PBMC-derived T cells.

FIG. 34 presents western blots to detect the indicated cytokine signaling events in primary murine T cells expressing G2R-2 or the single-chain G/IL-2Rβ (from G2R-1) versus mock-transduced cells. Arrow indicates the specific phospho-JAK2 band; other larger bands are presumed to be the result of cross-reactivity of the primary anti-phospho-JAK2 antibody with phospho-JAK1. β-actin and histone H3 serve as protein loading controls.

FIG. 35 presents a graph showing the results of a BrdU incorporation assay to assess cell cycle progression of 32D-IL-2Rβ cells expressing the indicated chimeric receptors (or non-transduced cells) in response to stimulation with no cytokine, IL-2 (300 IU/mL), WT G-CSF (30 ng/mL) or 130 G-CSF (30 ng/mL).

FIG. 36 presents graphs showing the results of BrdU incorporation assays to assess cell cycle progression of primary murine T cells expressing the indicated chimeric receptors (or non-transduced cells) in response to stimulation with no cytokine, IL-2, WT G-CSF or G-CSF variants 130, 304 or 307. A and B represent experimental replicates.

FIG. 37 presents western blots to detect the indicated cytokine signaling events in 32D-IL-2Rβ cells expressing the indicated chimeric receptor subunits (or non-transduced cells) in response to stimulation with no cytokine, IL-2, WT G-CSF or 130 G-CSF. β-actin and histone H3 serve as protein loading controls.

FIG. 38 presents A) western blots to detect the indicated cytokine signaling events in primary murine T cells expressing the indicated chimeric receptor subunits in response to stimulation with no cytokine, IL-2, WT G-CSF, 130 G-CSF or 304 G-CSF. β-actin and histone H3 serve as protein loading controls. B) Transduction efficiency of cells used in panel A, as assessed by flow cytometry with an antibody specific for the extracellular domain of the human G-CSF receptor.

FIG. 39 presents plots showing G-CSFR ECD expression by flow cytometry in primary human tumor-associated lymphocytes (TAL) transduced with the indicated chimeric receptor constructs. Live CD3+, CD56− cells were gated on CD8 or CD4, and G-CSFR ECD expression is shown for each population.

FIG. 40 presents graphs and images showing the expansion, proliferation and signaling of primary human tumor-associated lymphocytes (TAL) expressing G2R-3 versus non-transduced cells. A) Graph showing the results of a T-cell expansion assay, where cells were transduced with G2R-3-encoding lentivirus, washed, and re-plated in IL-2 (300 IU/ml), wild type G-CSF (100 ng/ml) or no cytokine. Live cells were counted every 3-4 days. Squares represent cells stimulated with IL-2. Triangles represent cells stimulated with G-CSF. Circles represent cells not stimulated with a cytokine. B) Western blot to assess intracellular signaling events. Cells were harvested from the expansion assay and stimulated with IL-2 (300 IU/ml) or wildtype G-CSF (100 ng/ml). Arrow indicates the specific phospho-JAK2 band at 125 kDa; larger bands are presumed to be the result of cross-reactivity of the primary anti-phospho-JAK2 antibody with phospho-JAK1. β-actin and histone H3 serve as protein loading controls. C) Graph showing the results of a BrdU incorporation assay to assess T-cell proliferation. Cells were harvested from the expansion assay, washed, and re-plated in IL-2 (300 IU/ml), wild type G-CSF (100 ng/ml) or no cytokine.

FIG. 41 presents graphs showing the fold expansion and G-CSFR ECD expression of primary human PBMC-derived T cells expressing G2R-3 with WT ECD versus non-transduced cells. A) Graph showing the results of a T-cell expansion assay, where cells were transduced with G2R-3-encoding lentivirus. On Day 1, WT G-CSF (100 ng/ml) or no cytokine (medium alone) were added to the culture. Thereafter, to Day 21, cells were replenished with medium containing WT G-CSF or no cytokine. On Day 21 of expansion, cells were washed and re-plated in WT G-CSF (100 ng/mL), IL-7 (20 ng/mL) and IL-15 (20 ng/mL), or no cytokine. Live cells were counted every 2-4 days. Squares represent cells stimulated with G-CSF. Triangles represent cells stimulated with G-CSF and re-plated in IL-7 and IL-15 on Day 21. Circles represent cells not stimulated with a cytokine. Diamonds represent cells stimulated with G-CSF, and re-plated in medium only on Day 21. B) Graph showing the expression of the G-CSFR ECD, as determined by flow cytometry, on Day 21 or 42 of expansion.

FIG. 42 presents graphs showing the intracellular signaling and immunophenotype of primary human PBMC-derived T cells expressing G2R-3 versus non-transduced cells. A) Western blot to assess intracellular signaling events. Cells were harvested from an expansion assay and stimulated with IL-2 (300 IU/ml) or wildtype G-CSF (100 ng/ml). β-actin serves as protein loading control. B, C) Representative flow cytometry plots and graphs showing the immunophenotype, assessed by flow cytometry, of cells expressing G2R-3 versus non-transduced cells on Day 42 of expansion.

FIG. 43 presents graphs showing the fold expansion of primary human PBMC-derived T cells expressing G2R-3 with 304 or 307 ECD versus non-transduced cells. A) Graph showing the results of a T-cell expansion assay, where cells were transduced with G2R-3 304 ECD-encoding lentivirus. B) Graph showing the results of a T-cell expansion assay, where cells were transduced with G2R-3 307 ECD-encoding lentivirus. C) Graph showing the results of a T-cell expansion assay with non-transduced cells. On Day 2 IL-2 (300 IU/mL), 304 G-CSF (100 ng/ml), 307 G-CSF (100 ng/mL) or no cytokine (medium alone) were added to the culture, as indicated, and replenished every two days thereafter. Live cells were counted every 3-4 days. Diamonds represent cells stimulated with 304 G-CSF. Squares represent cells stimulated with 307 G-CSF. Triangles represent cells stimulated with IL-2. Inverted triangles represent cells not stimulated with a cytokine.

FIG. 44 presents a graph showing the results of a BrdU incorporation assay to assess proliferation of primary human PBMC-derived T cells expressing G2R-3 with 304 or 307 ECD versus non-transduced cells. Cells were transduced with G2R-3 304 ECD- or 307 ECD-encoding lentivirus and expanded in the 304 or 307 G-CSF (100 ng/mL). Non-transduced cells were expanded in IL-2 (300 IU/mL). On Day 12 of expansion cells were washed, and re-plated in IL-2 (300 IU/ml), 130 G-CSF (100 ng/ml), 304 G-CSF (100 ng/ml), 307 G-CSF (100 ng/ml) or no cytokine.

FIG. 45 presents a graph showing G-CSFR ECD expression by flow cytometry in primary murine T cells transduced with the indicated chimeric receptor constructs.

FIG. 46 shows G-CSF-induced phosphorylation of STAT3 (detected by flow cytometry) in primary PBMC-derived human T cells expressing G21R-1 or G21R-2. Cells were subdivided (i.e., gated) into G-CSFR-positive (upper panels) or G-CSFR-negative (lower panels) populations.

FIG. 47 presents graphs and images showing G-CSF-induced biochemical signaling events in primary murine T cells expressing G21R-1 or G12R-1. A) Graph showing phosphorylation of STAT3 (detected by flow cytometry) in CD4+ or CD8+ cells transduced with G21R-1 and stimulated with no cytokine, IL-21 or G-CSF. B) Graph showing the percentage of cells staining positive for phospho-STAT3 after stimulation with no cytokine (black circles), IL-21 (squares) or WT G-CSF (gray circles). Live cells were gated on CD8 or CD4, and the percentage of phospho-STAT3-positive cells is shown for each population. C) Western blots to assess the indicated cytokine signaling events in cells expressing G21R-1 or G12R-1 and stimulated with IL-21, IL-12 or WT G-CSF. β-actin and histone H3 serve as protein loading controls.

FIG. 48 presents graphs and images showing proliferation, G-CSFR ECD expression and WT G-CSF-induced intracellular signaling events in primary murine T cells expressing G2R-2, G2R-3, G7R-1, G21/7R-1 and G27/2R-1, or mock-transduced T cells. A, B) Graphs showing the results of BrdU incorporation assays to assess T-cell proliferation. Cells were harvested, washed, and re-plated in IL-2 (300 IU/ml), wildtype G-CSF (100 ng/ml) or no cytokine. Panels A and B are experimental replicates. C) Graph showing G-CSFR ECD expression by flow cytometry in primary murine T cells transduced with the indicated chimeric receptor constructs. D) Western blots to assess the indicated cytokine signaling events in cells expressing G2R-2, G2R-3, G7R-1, G21/7R-1 and G27/2R-1, or mock-transduced T cells. Cells were stimulated with IL-2 (300 IU/mL), IL-7 (10 ng/mL), IL-21 (10 ng/mL), IL-27 (50 ng/mL) or G-CSF (100 ng/mL). β-actin and histone H3 serve as protein loading controls.

FIG. 49 presents graphs and images showing proliferation, G-CSFR ECD expression and G-CSF-induced biochemical signaling events in primary murine T cells expressing G21/2R-1, G12/2R-1 and 21/12/2R-1, or mock-transduced T cells. A, B) Graphs showing the results of BrdU incorporation assays to assess T-cell proliferation. Cells were harvested, washed, and re-plated in IL-2 (300 IU/ml), wild type G-CSF (100 ng/ml) or no cytokine. Panels A and B are experimental replicates. C) Graph showing G-CSFR ECD expression by flow cytometry in primary murine T cells transduced with the indicated chimeric receptor constructs. D) Western blots to assess the indicated cytokine signaling events in cells expressing G21/2R-1, G12/2R-1 and G21/12/2R-1 or mock-transduced T cells. Cells were stimulated with IL-2 (300 IU/mL), IL-21 (10 ng/mL), IL-12 (10 ng/mL) or G-CSF (100 ng/mL). β-actin and histone H3 serve as protein loading controls.

FIG. 50 presents graphs showing the fold expansion and G-CSFR ECD expression of primary human PBMC-derived T cells expressing G12/2R-1 with 134 ECD, versus non-transduced cells. A) Graph showing the results of a T-cell expansion assay, where cells were transduced with lentivirus encoding G12/2R-1 134 ECD and expanded in IL-2 (300 IU/mL), 130 G-CSF (100 ng/ml) or medium. Live cells were counted every 4-5 days. Squares represent cells not stimulated with a cytokine. Triangles represent cells stimulated with 130 G-CSF. Diamonds represent cells stimulated with IL-2. B) Graph showing the results of a T-cell expansion assay, where cells were transduced as in panel A. On Day 19 of expansion, cells were washed and re-plated in IL-2, 130 G-CSF or medium only. Live cells were counted every 4-5 days. Squares represent cells not stimulated with a cytokine. Light grey diamonds represent cells stimulated with 130 G-CSF. Dark grey diamonds represent cells stimulated with IL-2. Light grey inverted triangles represent cells initially stimulated with IL-2, and then re-plated in medium only on Day 19. Dark grey triangles represent cells initially stimulated with 130 G-CSF, and then re-plated in medium alone on Day 19. C) Graph showing the expression of the G-CSFR ECD, as determined by flow cytometry, on Day 4 or 16 of expansion.

FIG. 51 presents graphs showing the proliferation and immunophenotype of primary human PBMC-derived T cells expressing G12/2R-1 134 ECD, versus non-transduced cells. A) Graph showing the results of a BrdU incorporation assay to assess T-cell proliferation. Cells were harvested, washed, and re-plated in IL-2 (300 IU/ml), IL-2+IL-12 (300 IU/ml and 10 ng/mL, respectively), 130 G-CSF (300 ng/ml) or medium alone. B, C) Representative flow cytometry plots and graph showing the immunophenotype, assessed by flow cytometry, of cells expressing G12/2R-1 with 134 ECD, versus non-transduced cells, on Day 16 of expansion.

FIG. 52 presents graphs showing the fold expansion and proliferation of primary human PBMC-derived T cells expressing G12/2R-1 with 304 ECD, versus non-transduced cells. A) Graph showing the results of a T-cell expansion assay, where cells were transduced with lentivirus encoding G12/2R-1 134 ECD, and expanded in IL-2 (300 IU/mL), 130 G-CSF (100 ng/ml), 304 G-CSF (100 ng/ml) or medium alone. Non-transduced cells were cultured in IL-2, 130 G-CSF, 304 G-CSF or medium alone. Live cells were counted every 3-4 days. Inverted triangles represent cells not stimulated with cytokine. Triangles represent cells stimulated with IL-2. Circles represent cells stimulated with 130 G-CSF. Diamonds represent cells stimulated with 304 G-CSF. B) Cells were harvested from the expansion assay on Day 12, and were washed and re-plated in IL-2 (300 IU/ml), 130 G-CSF (300 ng/ml), 304 G-CSF (100 ng/ml), 307 G-CSF (100 ng/ml) or medium alone.

FIG. 53 presents western blots to detect the indicated cytokine signaling events in primary PBMC-derived T cells expressing G2R-3 with 304 ECD, G12/2R-1 with 304 ECD, or non-transduced T cells. Cells were harvested from the expansion assay and stimulated with 304 G-CSF (100 ng/mL), IL-2 (300 IU/mL), IL-2 and IL-12 (10 ng/mL), or medium alone, as indicated. Black arrows and small outcropped panel on the right indicate the molecular weight markers at 115 kDa and 140 kDa from a protein ladder. β-actin and histone H3 serve as protein loading controls.

FIG. 54 presents a size exclusion UV trace of refolded 130a1 G-CSF (labelled as GCSF_130a1) and the corresponding SDS PAGE purity gel stained with Coomassie blue. Refolded 130a1 G-CSF eluted at the expected volume relative to standards of known molecular weight.

FIG. 55 presents the results of BrdU incorporation assays to assess proliferation of (A, B, C) OCI-AML1 cells, which naturally express wild-type (WT) human G-CSFR, or (D, E) 32D clone 3 cells, which naturally express WT murine G-CSFR. Cells were stimulated with the following cytokines: (A) WT G-CSF or the G-CSF variants 130, 130a1, 130b1, 130a2, or 130b2; (B) WT G-CSF or the G-CSF variants 130, 130a1, or 130b1, or medium alone (no added cytokine); (C) WT G-CSF or the G-CSF variants 130, 130a1, or 130a1b1, or medium alone; (D) WT G-CSF or the G-CSF variants 130, 130a1, or 130b1, or medium alone; and (E) WT G-CSF or the G-CSF variants 130, 130a1, or 130a1b1, or medium alone. Solid squares represent WT G-CSF; solid triangles represent 130 G-CSF; solid inverted triangles represent 130a1 G-CSF; open circles represent 130a2 G-CSF; solid diamonds represent 130b1 G-CSF; open squares represent 130bd G-CSF; and open diamonds represent 130a1b1 G-CSF.

FIG. 56 presents the results of BrdU incorporation assays to assess proliferation of PBMC-derived human T cells expressing G12/2R-1 134 ECD. Cells were stimulated with (A) medium alone or medium plus IL-2, IL-2+IL-12, or the G-CSF variants 130, 130a1, 130b1, 130a2 or 130b2; or (B) medium alone or medium plus IL-2, IL-2+IL-12 or the G-CSF variants 130 or 130a1b1. Results are shown for T cells expressing G12/2R-1 134 ECD (i.e., T cells that were G-CSFR+ by flow cytometry).

FIG. 57 presents the results of an in vivo experiment to determine the safety and efficacy of adoptive cell therapy with tumor-specific CD8 T cells (Thy1.1+ OT-I T cells) that were retrovirally transduced to express G2R-3 134 ECD and then infused into syngeneic, immune competent mice bearing established mammary tumors (NOP23 tumor line). Beginning on the day of T cell infusion (day 0), mice were randomized and treated with vehicle (n=4 animals) or 130a1 G-CSF (10 μg/dose; n=4 animals) daily for 14 days, followed by every other day for 14 days, for a total of 21 doses. (A) Tumor area (length×width) from day −2 to 80. Each line represents results from an individual animal. (B) Higher resolution view of tumor area from day −2 to 20. (C) Kaplan-Meier plot showing the percent of animals alive from day 0 to 80. (D) Expansion of OT-I T cells in peripheral blood shown as the average percentage of Thy1.1+(OT-I) cells relative to all CD8⁺ T cells (mean+/−SEM). (E) Percentage of neutrophils relative to all CD45+ cells in peripheral blood (mean+/−SEM). (F) Percentage of eosinophils relative to all CD45+ cells in peripheral blood (mean+/−SEM). (G) Percentage of monocytes relative to all CD45+ cells in peripheral blood (mean+/−SEM). Gray circles represent vehicle-treated animals and black triangles represent 130a1 G-CSF-treated animals.

FIG. 58 presents the results of an in vivo experiment to determine the safety and efficacy of adoptive cell therapy with tumor-specific CD8 T cells (Thy1.1+ OT-I T cells) that were retrovirally transduced to express G12/2R-1 134 ECD and then infused into syngeneic, immune competent mice bearing established mammary tumors (NOP23 tumor line). Beginning on the day of T cell infusion (day 0), mice were randomized and treated with vehicle (n=4 animals) or 130a1 G-CSF (10 μg/dose; n=3 animals) daily for 14 days, followed by every other day for 14 days, for a total of 21 doses. (A) Tumor area (length×width) from day −2 to 80. Each line represents results from an individual animal. (B) Higher resolution view of tumor area from day −2 to 20. (C) Kaplan-Meier plot showing the percent of animals alive from day 0 to 80. (D) Expansion of OT-I T cells in peripheral blood shown as the average percentage of Thy1.1+(OT-I) cells relative to all CD8⁺ T cells (mean+/−SEM). (E) Percentage of neutrophils relative to all CD45+ cells in peripheral blood (mean+/−SEM). (F) Percentage of eosinophils relative to all CD45+ cells in peripheral blood (mean+/−SEM). (G) Percentage of monocytes relative to all CD45+ cells in peripheral blood (mean+/−SEM). Gray circles represent vehicle-treated animals and black triangles represent 130a1 G-CSF-treated animals.

FIG. 59 presents the results for three control groups from the experiments shown in FIGS. 4 and 5. Tumor-specific CD8 T cells (Thy1.1+ OT-I T cells) underwent a mock retroviral transduction procedure and were then infused into syngeneic, immune competent mice bearing established mammary tumors (NOP23 tumor line). Beginning on the day of T cell infusion (day 0), mice were randomized and treated with vehicle (n=4 animals), IL-2 (30,000 IU/dose; n=5 animals) or 130a1 (10 μg/dose; n=5 animals) daily for 14 days, followed by every other day for 14 days, for a total of 21 doses. (A) Tumor area (length×width) from day −2 to 80. Each line represents results from an individual animal. (B) Higher resolution view of tumor area from day −2 to 20. (C) Kaplan-Meier plot showing the percent of animals alive from day 0 to 80. (D) Expansion of OT-I T cells in peripheral blood shown as the average percentage of Thy1.1+(OT-I) cells relative to all CD8⁺ T cells (mean+/−SEM). (E) Percentage of neutrophils relative to all CD45+ cells in peripheral blood (mean+/−SEM). (F) Percentage of eosinophils relative to all CD45+ cells in peripheral blood (mean+/−SEM). (G) Percentage of monocytes relative to all CD45+ cells in peripheral blood (mean+/−SEM). Gray circles represent vehicle-treated animals; gray squares represent IL-2-treated animals; and black triangles represent 130a1 G-CSF-treated animals.

FIG. 60 presents schematics of wild type cytokine receptor subunits and additional chimeric receptor designs.

FIG. 61 presents flow cytometry data showing cell surface expression of the G-CSFR 134 ECD on human PBMC-derived CD3⁺ T cells transduced with G4R 134 ECD. Non-transduced T cells served as a negative control.

FIG. 62 presents graphs showing the expansion (fold change in cell number) of human PBMC-derived T cells expressing (A) G4R 134 ECD cultured with medium alone or medium plus IL-2, 130a1 G-CSF or 130a1 G-CSF+IL-2. (B) Results for non-transduced T cells cultured with medium alone or medium plus IL-2 or 130a1 G-CSF+307 G-CSF. The 307 G-CSF variant was included in this latter condition to serve as a control for another arm of this experiment (not shown); we previously established, and confirm here, that neither 130a1 G-CSF nor 307 G-CSF induces proliferation of non-transduced T cells. Diamonds represent cells cultured with 130a1 G-CSF (panel A) or 130a1 G-CSF+307 G-CSF (panel B). Triangles represent cells cultured with IL-2. Light-grey circles represent cells cultured with 130a1 G-CSF and IL-2. Black circles represent cells cultured with medium alone (no added cytokine).

FIG. 63 presents graphs showing the results of a BrdU incorporation assay to assess proliferation of primary human T cells expressing (A) G4R 134 ECD versus (B) non-transduced T cells. Cells were stimulated with medium alone, IL-2, IL-4, IL2+IL-4, 130a1 G-CSF, or 130a1 G-CSF+IL-2. Data is presented separately for the CD4+ and CD8⁺ T cell subsets. Data in panel A is gated on T cells expressing G4R 134 ECD (detected using an antibody against human G-CSFR).

FIG. 64 presents western blots to detect the indicated biochemical signaling events in human PBMC-derived T cells expressing G4R 134 ECD versus non-transduced cells. Cells were stimulated with IL-2, IL-4 or 130a1 G-CSF. β-actin and histone H3 served as protein loading controls.

FIG. 65 presents flow cytometry data showing cell surface expression of the G-CSFR 134 ECD on human PBMC-derived CD3⁺ T cells transduced with a lentivirus encoding G6R 134 ECD. Non-transduced T cells served as a negative control.

FIG. 66 presents graphs showing the expansion (fold change in cell number) of human PBMC-derived T cells expressing (A) G6R 134 ECD or (B) non-transduced T cells cultured in medium alone or medium with IL-2, 130a1 G-CSF, or 130a1 G-CSF+IL-2. Diamonds represent cells stimulated with 130a1 G-CSF. Triangles represent cells stimulated with IL-2. Light-grey circles represent cells stimulated with 130a1 G-CSF+IL-2. Black circles represent cells stimulated with medium alone (no added cytokine).

FIG. 67 presents graphs showing the results of a BrdU incorporation assay to assess proliferation of PBMC-derived human T cells expressing (A) G6R 134 ECD compared to (B) non-transduced T cells. Cells were stimulated with medium alone or medium plus IL-2, IL-6, IL-2+IL-6, 130a1 G-CSF, or 130a1 G-CSF+IL-2. Data is shown separately for the CD4+ and CD8⁺ T cell subsets. Data in panel A was gated on T cells expressing G4R 134 ECD (detected using an antibody against human G-CSFR).

FIG. 68 presents western blots to detect the indicated biochemical signaling events in human PBMC-derived T cells expressing G6R 134 ECD versus non-transduced cells. Cells were stimulated with IL-2, IL-6 or 130a1 G-CSF. Histone H3 served as a protein loading control. Note that on the P-STAT3 image a dark, higher molecular band appears in the IL-2-stimulated conditions (especially in the non-transduced T cells). This is remnant signal from a previous probing of this membrane with phospho-STAT5 antibody. The lower molecular weight band (arrow) represents phospho-STAT3.

FIG. 69 presents flow cytometry data showing cell surface expression of the G-CSFR 134 ECD on human PBMC-derived CD3⁺ T cells transduced with GEPOR 134 ECD. Non-transduced T cells served as a negative control.

FIG. 70 presents graphs showing the expansion (fold change in cell number) of primary human PBMC-derived T cells expressing (A) GEPOR 134 ECD or (B) non-transduced T cells cultured in medium alone or medium with IL-2, 130a1 G-CSF, or 130a1 G-CSF+IL-2. For non-transduced T cells, 307 G-CSF was added to the 130a1 G-CSF condition to serve as a control for another arm of this experiment (not shown); we previously established, and confirm here, that neither 130a1 G-CSF nor 307 G-CSF induces proliferation of non-transduced T cells. Diamonds represent cells cultured in 130a1 G-CSF+/−307 G-CSF. Triangles represent cells cultured in IL-2. Light-grey circles represent cells cultured in 130a1 G-CSF+IL-2. Black circles represent cells cultured in medium alone.

FIG. 71 presents western blots to detect the indicated biochemical signaling events in human PBMC-derived T cells expressing GEPOR 134 ECD versus non-transduced cells. Cells were stimulated with medium alone or medium plus IL-2 or 130a1 G-CSF. 3-actin and histone H3 served as protein loading controls.

FIG. 72 presents flow cytometry data showing cell surface expression of the G-CSFR 134 ECD on human PBMC-derived CD3⁺ T cells transduced with GIFNAR 134 ECD. Non-transduced T cells served as a negative control.

FIG. 73 presents graphs showing the expansion (fold change in cell number) of primary human PBMC-derived T cells that were (A) non-transduced or (B) transduced to express GIFNAR 134 ECD and cultured in medium alone or medium with IL-2, 130a1 G-CSF, or 130a1 G-CSF+IL-2. Diamonds represent cells cultured in 130a1 G-CSF. Triangles represent cells cultured in IL-2. Light-grey circles represent cells cultured in 130a1 G-CSF+IL-2. Black circles represent cells cultured in medium alone.

FIG. 74 presents western blots to detect the indicated biochemical signaling events in human PBMC-derived T cells expressing GIFNAR 134 ECD versus non-transduced cells. Cells were stimulated with medium alone or medium plus IL-2, IFNα or 130a1 G-CSF. β-actin and histone H3 served as protein loading controls.

FIG. 75 presents flow cytometric data showing Myc-tag and Flag-tag expression on the surface of human PBMC-derived T cells transduced with (A) GIFNGR-1 307 ECD, (B) G2R3 134 ECD, (C) GIFNGR-1 307 ECD and G2R3 134 ECD or (D) non-transduced T cells. G2R3 134 ECD was tagged at its N-terminus with a Myc epitope (EQKLISEEDL) (SEQ ID NO.227) and GIFNGR-1 307 ECD was tagged at its N-terminus with a Flag epitope (DYKDDDDK) SEQ ID NO.228); the epitope tags aid detection by flow cytometry and do not impact the function of the receptors. Plots were gated on CD3+ cells.

FIG. 76 presents flow cytometric data showing Myc-tag and Flag-tag expression on the surface of human PBMC-derived T cells transduced with (A) GIFNGR-2 307 ECD, (B) G2R3 134 ECD, (C) GIFNGR-2 307 ECD and G2R3 134 ECD, or (D) non-transduced T cells. G2R3 134 ECD was tagged at its N-terminus with a Myc epitope (EQKLISEEDL) SEQ ID NO.227), and GIFNGR-2 307 ECD was tagged at its N-terminus with a Flag epitope (DYKDDDDK) (SEQ ID NO.228); the epitope tags aid detection by flow cytometry and do not impact the function of the receptors. Plots were gated on CD3+ cells.

FIG. 77 presents graphs showing the expansion (fold change in cell number) of primary human PBMC-derived T cells that were (A) non-transduced, (B) transduced to express GIFNGR-1 307 ECD, or (C) co-transduced to express GIFNGR-1 307 ECD and G2R-3 134 ECD. T cells were cultured in medium alone or medium with the indicated combinations of IL-2, 130a1 G-CSF and 307 G-CSF. Diamonds and light-grey circles represent cells cultured in the indicated combinations of 130a1 G-CSF, 307 G-CSF and IL-2. Triangles represent cells cultured in IL-2. Black circles represent cells cultured in medium alone.

FIG. 78 presents graphs showing the expansion (fold change in cell number) of primary human PBMC-derived T cells that were (A) non-transduced, (B) transduced to express GIFNGR-2 307 ECD, or (C) co-transduced to express GIFNGR-2 307 ECD and G2R-3 134 ECD. T cells were cultured in medium alone or medium with the indicated combinations of IL-2, 130a1 G-CSF and 307 G-CSF. Diamonds and light-grey circles represent cells cultured in the indicated combinations of 130a1 G-CSF, 307 G-CSF and IL-2. Triangles represent cells cultured in IL-2. Black circles represent cells cultured in medium alone.

FIG. 79 presents graphs showing the results of a BrdU incorporation assay to assess proliferation of human PBMC-derived T cells expressing G2R-3 134 ECD and/or GIFNGR-1 307 ECD. Data are shown for (A) non-transduced T cells or (B) CD4+ and (C) CD8⁺ T cells expressing the indicated chimeric receptors (see X-axis). Cells were stimulated with the indicated combinations of IL-2, IFNγ, 130a1 G-CSF, 307 G-CSF or medium alone.

FIG. 80 presents graphs showing the results of a BrdU incorporation assay to assess proliferation of human PBMC-derived T cells expressing G2R-3 134 ECD and/or GIFNGR-2 307 ECD. Data are shown for (A) non-transduced T cells or (B) CD4+ and (C) CD8+ T cells expressing the indicated chimeric receptors (see X-axis). Cells were stimulated with the indicated combinations of IL-2, IFNγ, 130a1 G-CSF, 307 G-CSF or medium alone.

FIG. 81 presents western blots to detect the indicated biochemical signaling events in human PBMC-derived T cells expressing GIFNGR-1 307 ECD or GIFNGR-2 307 ECD versus non-transduced cells. Cells were stimulated with medium alone or medium plus IL-2, IFNγ or 307 G-CSF. β-actin and histone H3 served as protein loading controls.

FIG. 82 presents flow cytometry data showing cell surface expression of G2R-3 134 ECD and a mesothelin-specific CAR on human PBMC-derived CD3⁺ T cells transduced with the following mono- or bi-cistronic lentiviral constructs: (A) Non-transduced T cells (negative control); (B) mesothelin CAR alone; (C) G2R-3 134 ECD alone; (D) CAR_T2A_G2R-3-134 ECD, which has gene segments in the following order: mesothelin CAR, T2A site and G2R-3 134 ECD; and (E) G2R-3-134 ECD_T2A_CAR, which has gene segments in the following order: mesothelin CAR, T2A site and G2R-3 134 ECD.

FIG. 83 presents flow cytometry data showing cell surface expression of G12/2R-1 134 ECD and a mesothelin-specific CAR on human PBMC-derived CD3⁺ T cells transduced with the following mono- or bi-cistronic lentiviral constructs: (A) Non-transduced T cells (negative control); (B) mesothelin CAR alone; (C) G2R-3 134 ECD alone; (D) CAR_T2A_G12/2R-1-134 ECD, which has gene segments in the following order: mesothelin CAR, T2A site and G12/2R-1 134 ECD; and (E) G12/2R-1-134 ECD_T2A_CAR, which has gene segments in the following order: mesothelin CAR, T2A site and G12/2R-1 134 ECD.

FIG. 84 presents the results of BrdU incorporation assays to assess proliferation of PBMC-derived human T cells modified as follows: (A) non-transduced or expressing mesothelin CAR only; or (B) expressing G2R3 134 ECD, or bicistronic CAR_T2A_G2R3-134 ECD, or bicistronic CAR_T2A_G12/2R-1-134 ECD, or bicistronic G12/2R-1-134ECD_T2A_CAR constructs. Cells were stimulated with medium alone or medium plus IL-2 or 130a1 G-CSF. Results in panel B are shown for T cells that were positive for G-CSFR ECD expression by flow cytometry.

FIG. 85 presents the results of in vitro co-culture assays to assess the functional properties of a mesothelin CAR in PBMC-derived human CD4⁺ T cells expressing: (A) mesothelin CAR only; (B) G12/2R-1 134 ECD only, or the following bicistronic constructs containing a mesothelin CAR: (C) CAR_T2A_G2R3-134 ECD, (D) G2R3-134 ECD_T2A_CAR, (E) CAR_T2A_G12/2R-1-134 ECD or (F) G12/2R-1-134ECD_T2A_CAR. Cells were left non-stimulated or stimulated with OVCAR3 cells for 13 hours, followed by intracellular flow cytometry to detect expression of the cytokines IFNγ, TNFα and IL-2, as well as the surface molecules CD69 and CD137. Results in panels A, C, D, E and F are gated on cells expressing the mesothelin CAR.

FIG. 86 is a schematic of native cytokine receptors, including IL-7Rα, IL-2Rβ, gp130, IL-21R, IL-12Rβ2 and IL-4Rα.

FIG. 87 is a schematic of 7/2R-1, 7/2R-2, 7/2/12R-1, 7/2/12R-2, 7/21R-1, 7/21R-2, 7/7/21R-1, 7/7/21R-2, 7/2/21R-1, 7/2/21R-27/2/12/21R-1, 7/2/12/21R-2, 7/2/12/21R-3, 7/2/12/21R-4, 7/4R-1, 7/4R-2, 7/6R-1, and 7/6R-2 receptor subunit designs.

FIG. 88 presents graphs showing human CD127 (IL-7Rα) and Flag-tag expression assessed by flow cytometry in (A) primary human PBMC-derived T cells transduced with 7/2R-1, (B) non-transduced T cells, or (C) T cells transduced with 7/2R-1 but stained as a fluorescence-minus-one (FMO) control (i.e., the antibody to CD127 was left out of the antibody staining panel). 7/2R-1 was tagged at its N-terminus with a Flag epitope; the epitope tag aids detection by flow cytometry and does not impact the function of the receptors. Receptor expression was analyzed 12 days after lentiviral transduction.

FIG. 89 presents graphs showing the expansion (fold change in cell number) of primary human PBMC-derived T cells (A) expressing 7/2R-1 versus (B) non-transduced T cells and cultured with IL-7, IL-7+IL-15, or no cytokine. Diamonds represent cells stimulated with IL-7. Triangles represent cells stimulated with IL-7 and IL-15. Circles represent cells cultured in medium alone (no added cytokine).

FIG. 90 presents graphs showing the results of a BrdU incorporation assay to assess proliferation of primary human T cells expressing (A) 7/2R-1 versus (B) non-transduced T cells. Cells were stimulated with IL-7, IL-2, IL-2+IL-7, or medium alone (no added cytokine). Results are shown separately for the CD4+ and CD8+ T cell subsets.

FIG. 91 presents western blots to detect the indicated cytokine signaling events in human primary T cells expressing 7/2R-1 versus non-transduced T cells. β-actin and histone H3 serve as protein loading controls. Cells were stimulated with IL-7, IL-2, IL-2+IL-7, or medium alone (no added cytokine).

FIG. 92 presents an SDS-PAGE gel to detect unmodified 130a1 G-CSF, and pegylated forms of 130a1 G-CSF (PEG20k_G-CSF_130a1) and 307 G-CSF (PEG20k_G-CSF_307). Gel was stained with Coomassie brilliant blue.

FIG. 93 presents the results of an in vivo experiment to determine the safety and efficacy of adoptive cell therapy with tumor-specific CD8 T cells (Thy1.1+ OT-I T cells) that were retrovirally transduced to express G4R 134 ECD and then infused into syngeneic, immune competent mice bearing established mammary tumors (NOP23 tumor line). Beginning on the day of T cell infusion (day 0), mice were randomized and treated with vehicle (n=3 animals) or 130a1 G-CSF (10 μg/dose; n=3 animals) daily for 14 days, followed by every other day for 14 days, for a total of 21 doses. (A) Tumor area (length×width) from day 0 to 46. Each line represents results from an individual animal. (B) Expansion of OT-I T cells in peripheral blood shown as the mean percentage of Thy1.1+(OT-I) cells relative to all CD8+ T cells (mean+/−standard deviation [SD]). (C) Percentage of OT-I T cells (Thy1.1+) exhibiting a T effector memory (Tem; CD44+CD62L−) phenotype (mean+/−SD). (D) Percentage of host (Thy1.1−) CD8+ T cells exhibiting a Tem phenotype (mean+/−SD). (E) Percentage of OT-I T cells (Thy1.1+) expressing Programmed Death-1 (PD-1) (mean+/−SD). (F) Percentage of host (Thy1.1−) CD8+ T cells expressing PD-1 (mean+/−SD). (G) Percentage of host (Thy1.1−) CD3+ T cells relative to all CD45+ cells in peripheral blood (mean+/−SD). (H) Percentage of host (Thy1.1−) CD19+ B cells relative to all CD45+ cells in peripheral blood (mean+/−SD). Gray circles represent vehicle-treated animals and black triangles represent 130a1 G-CSF-treated animals.

FIG. 94 presents the results of an in vitro experiment to compare pegylated versus non-pegylated versions of G-CSF (wildtype, 130a1 and 307) for the ability to stimulate proliferation of cells expressing G2R-3 134 ECD or the native G-CSF receptor. Data is presented as the mean and standard deviation for duplicate wells. (A) Human PBMC-derived CD4 and CD8 T cells expressing G2R-3 134 ECD were stimulated with the indicated cytokines: human IL-2 (Proleukin, 300 IU/ml, positive control), medium alone (negative control), or the indicated concentrations of wildtype G-CSF, pegylated wildtype G-CSF, 130a1 G-CSF, or pegylated 130a1 G-CSF. Cells were cultured for 48 hours and assessed by BrdU incorporation assay. Results are shown for T cells that were positive for human G-CSFR. (B) A similar experiment was performed using the human myeloid cell line OCI-AML-1, which naturally expresses the wildtype human G-CSF receptor. Cells were stimulated with the indicated cytokines: human GM-CSF (20 ng/ml, positive control), medium alone (negative control), or the indicated concentrations of wildtype G-CSF, pegylated wildtype G-CSF, 130a1 G-CSF, pegylated 130a1 G-CSF, 307 G-CSF, or pegylated 307 G-CSF. Cells were cultured for 48 hours and assessed by BrdU incorporation assay.

FIG. 95 presents the results of an in vitro Western blot experiment comparing the ability of pegylated versus non-pegylated versions of 130a1 G-CSF to induce the indicated biochemical signaling events in human PBMC-derived T cells expressing G2R-3 134 ECD. T cells were stimulated for 20 min with human IL-2 (Proleukin, 300 IU/ml) or the indicated concentrations (in ng/ml) of non-pegylated or pegylated (PEG) versions of 130a1 G-CSF. Histone H3 and Total S6 serve as gel loading controls.

FIG. 96 presents the results of an in vivo experiment comparing the potency of pegylated (PEG) versus non-pegylated versions of 130a1 G-CSF given at daily, every three days, or weekly intervals. Tumor-specific CD8 T cells (Thy1.1+ OT-I T cells) were retrovirally transduced to express G2R-3 134 ECD and then infused into syngeneic, immune competent mice bearing established mammary tumors (NOP23 tumor line). Tumor size (length×width) is plotted over a 46-day period. Each line represents results from an individual animal. The right panels are an expanded version of the left panels. Beginning on the day of T cell infusion (day 0), mice were randomized and treated with vehicle, 130a1 G-CSF, or PEG-130a1 G-CSF as indicated. (A, B) The “daily” dosing cohort received the indicated cytokines (or vehicle) daily for 14 days, followed by every other day for 14 days, for a total of 21 doses. (C, D) The “every three days” dosing cohort received the indicated cytokines every three days for a total of 9 doses. (E,F) The “weekly” dosing cohort received the indicated cytokines every seven days for a total of 4 doses.

FIG. 97 shows the expansion and effector memory (Tem) phenotype of OT-I T cells in peripheral blood at the indicated time points from the experiment shown in FIG. 96. Left panels show the percentage of Thy1.1+(OT-I) T cells relative to all CD8⁺ T cells (mean+/−SD). Right panels show the percentage of Thy1.1+(OT-I) T cells that have a Tem (CD44+CD62L−) phenotype (mean+/−SD). (A, D) “Daily” dosing cohort. (B, E) “Every three days” dosing cohort. (C,F) “Weekly” dosing cohort.

FIG. 98 shows the percentage of the indicated immune cell subsets (relative to all CD45+ cells) in peripheral blood at the indicated time points from the “every three days” cohort from the experiment shown in FIGS. 102 and 103. Mean and standard deviation are plotted. (A) Neutrophils (CD3−, CD19−, NK1.1−, CD11b+, CD11c−, Ly6G+). (B) Monocytes (CD3−, CD19−, NK1.1−, CD11b+, CD11c−, Ly6G−, SSC-low (Ly6C+ or Ly6C−). (C) Eosinophils (CD3−, CD19−, NK1.1−, CD11b+, CD11c−, Ly6G−, SSC-high). (D) CD3⁺ T cells. (E) CD19+ B cells. (F) NK1.1+ Natural Killer cells.

FIG. 99 shows in vitro data from an IL-18 Controlled Paracrine Signaling (CPS) experiment. Human PBMC-derived T cells were transduced with either a bi-cistronic vector encoding a mesothelin-specific CAR, T2A site, and G12/2R-1 134 ECD (Meso CAR_G12/2R-1), or a tri-cistronic vector encoding a mesothelin-specific CAR, T2A site, G12/2R-1 134 ECD and human IL-18 (Meso CAR_G12/2R-1+h18). (A) T cells were assessed by flow cytometry for expression of the mesothelin CAR (X-axis) and G12/2R-1 134 ECD (Y-axis). (B) An in vitro BrdU assay was used to assess T cell proliferation in response to medium alone; human IL-2 (Proleukin, 300 IU/ml); human IL-18 (100 ng/ml); human IL-2+ human IL-12 (20 ng/ml); IL-2+IL-12+IL-18; or 130a1 G-CSF (100 ng/ml). (C) ELISA was used to assess human IL-18 levels in culture supernatants from T cells stimulated for 48 hours with media alone; human IL-2 (Proleukin, 300 IU/ml)+ human IL-12 (20 ng/ml); or 130a1 G-CSF (100 ng/ml).

FIG. 100 shows the results of an in vitro IL-18 CPS experiment with murine T cells. Mouse CD4 and CD8 T cells were transduced with either a mono-cistronic vector encoding G12/2R-1 134 ECD, or a bi-cistronic vector encoding G12/2R 134 ECD, T2A site and murine IL-18 (G12/2R-1 134 ECD+m18). (A) T cells were assessed by flow cytometry for expression of the G12/2R-1 134 ECD. (B) An in vitro BrdU assay was used to assess T cell proliferation in response to medium alone; human IL-2 (Proleukin, 300 IU/ml); murine IL-18 (100 ng/ml); human IL-2+murine IL-12 (10 ng/ml); IL-2+IL-12+IL-18; or 130a1 G-CSF (100 ng/ml). (C) ELISA was used to assess murine IL-18 levels in culture supernatants from T cells stimulated for 48 hours with medium alone; human IL-2 (Proleukin, 300 IU/ml)+murine IL-12 (10 ng/ml); or 130a1 G-CSF (100 ng/ml).

FIG. 101 shows the results of an in vitro IL-18 CPS experiment with murine OT-I T cells. OT-I T cells were transduced with retroviral vectors encoding G2R-2 134 ECD, G2R-3 134 ECD, G12/2R-1 134 ECD or G12/2R-1 134 ECD+m18 (abbreviated as G12/2R/18C in the figure). To detect secreted cytokines, T cells were washed five days later and cultured for 48 hours in medium alone (negative control) or medium with human IL-2 (Proleukin 300 IU/ml), human IL-2+murine IL-12 (10 ng/ml), or pegylated (Peg) 130a1 (100 ng/ml), followed by a multiplex assay to detect 32 cytokines. Results are shown for (A) murine IL-18, and (B) murine IFN-gamma. Results for other cytokines are shown in Table 35.

FIG. 102 presents the results of an in vivo experiment comparing the properties of G7R-1, G2R-2, G12/2R-1, and G12/2R-1+m18 (abbreviated as G12/2R/18C) (all with the 134 ECD) in OT-I T cells in the NOP23 mammary tumor model. Tumor-specific CD8 T cells (Thy1.1+ OT-I T cells) were retrovirally transduced to express G7R-1, G2R-2, G12/2R-1 or G12/2R-1+m18 (all with the 134 ECD) and then infused into syngeneic, immune competent mice bearing established NOP23 mammary tumors. Beginning on the day of T cell infusion (day 0), mice were randomized to receive vehicle or pegylated (PEG) 130a1 G-CSF (10 μg/dose) on a weekly basis for a total of four treatments. (A-D) Flow cytometry results showing receptor (G-CSFR) expression on transduced OT-I T cells on the day of T cell infusion (Day 0). (E-H) Tumor size (length×width) over a 50-day period. Each line represents an individual animal. (I-L) Expansion and persistence of Thy1.1+ OT-I cells in serial blood samples. (M-P) Percentage of Thy1.1+ OT-I T cells exhibiting a T effector memory (Tem) phenotype (CD44+CD62L−) in blood.

FIG. 103 presents the results of an in vivo experiment in the NOP23 mammary tumor model comparing different doses of pegylated (PEG) 130a1 G-CSF, or wild type IL-2. Thy1.1+ OT-I T cells expressing G2R-3 134 ECD were infused into syngeneic, immune competent mice bearing established tumors. Beginning on the day of T cell infusion (day 0), mice were randomized to six cytokine treatment groups: vehicle alone (shown in all panels); PEG-130a1 G-CSF 2 μg/dose, four weekly doses (panels A, F, K, P); (3) PEG-130a1 G-CSF 0.4 μg/dose, four weekly doses (panels B, G, L, Q); (4) PEG-130a1 G-CSF 0.08 μg/dose, four weekly doses (panels C, H, M, R); (5) PEG-130a1 G-CSF four daily doses of 0.1 μg followed by three weekly doses of 0.4 μg (panels D, I, N, S); or (6) human IL-2 (Proleukin) at 30,000 IU/day for 14 days followed by 30,000 IU every two days for 14 days (panels E, J, O, T). Serial blood samples were analyzed by flow cytometry for the following parameters: (A-E) Percentage of Thy1.1+ OT-I cells relative to all CD8⁺ T cells (mean+/−SD). (F-J) Percentage of Thy1.1+ OT-I cells with a T effector memory (Tem) phenotype (CD44+CD62L−) (mean+/−SD). (K-O) Percentage of neutrophils (CD3−, CD19−, NK1.1−, CD11b+, CD11c−, Ly6G+) relative to all CD45+ cells (mean+/−SD). (P-T) Percentage of eosinophils (CD3−, CD19−, NK1.1−, CD11b+, CD11c−, Ly6G−, SSC-high) relative to all CD45+ cells (mean+/−SD).

### DETAILED DESCRIPTION

Briefly, and as described in more detail below, described herein are methods and compositions for selective activation of cells using variant cytokine receptor and cytokine pairs, wherein the cytokine receptors comprise a variant extracellular domain (ECD) of granulocyte-colony stimulating factor receptor (G-CSFR). In certain embodiments, the methods and compositions described herein are useful for exclusive activation of cells for adoptive cell transfer (ACT) therapy. Thus, included herein are methods for producing cells expressing variant receptors that are selectively activated by a cytokine that does not bind its native receptor. Also disclosed herein, are methods of treating a subject in need thereof, comprising administering to the subject a cell expressing receptors comprising a variant ECD of G-CSFR and co-administering a variant of G-CSF that binds to the variant ECD of G-CSFR. In certain aspects, the compositions and methods described herein address an unmet need for the selective activation of cells for adoptive cell transfer methods and may reduce or eliminate the need for immuno-depletion of a subject prior to adoptive cell transfer or for administering broad-acting stimulatory cytokines such as IL-2.

### Definitions

Terms used in the claims and specification are defined as set forth below unless otherwise specified.

The term “treatment” refers to any therapeutically beneficial result in the treatment of a disease state, e.g., a cancer disease state, lessening in the severity or progression, remission, or cure thereof.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to selectively activate a receptor expressed on a cell.

The term “therapeutically effective amount” is an amount that is effective to ameliorate a symptom of a disease.

The term “operatively linked” refers to nucleic acid or amino acid sequences that are placed into a functional relationship with another nucleic acid or amino acid sequence, respectively. Generally, “operatively linked” means that nucleic acid sequences or amino acid sequences being linked are contiguous, and, in the case of a secretory leader, contiguous and in reading phase.

As used herein, the term “extracellular domain” (ECD) refers to the domain of a receptor (e.g., G-CSFR) that, when expressed on the surface of a cell, is external to the plasma membrane. In certain embodiments the ECD of G-CSFR comprises at least a portion of SEQ ID NO. 2 or SEQ ID NO. 7.

As used herein, the term “intracellular domain” (ICD) refers to the domain of a receptor that is located within the cell when the receptor is expressed on a cell surface.

As used herein, the term “transmembrane domain” (TMD or TM) refers to the domain or region of a cell surface receptor that is located within the plasma membrane when the receptor is expressed on a cell surface.

The term “cytokine” refers to small proteins (about 5-20 kDa) that bind to cytokine receptors and can induce cell signaling upon binding to and activation of a cytokine receptor expressed on a cell. Examples of cytokines include, but are not limited to: interleukins, lymphokines, colony stimulating factors and chemokines.

The term “cytokine receptor” refers to receptors that bind to cytokines, including type 1 and type 2 cytokine receptors. Cytokine receptors include, but are not limited to, G-CSFR, IL-2R (Interleukin-2 receptor), IL-7R (Interleukin-7 receptor), IL-12R (Interleukin-12 Receptor), and IL-21R (Interleukin-21 Receptor).

The term “chimeric receptors,” as used herein, refers to a transmembrane receptor that is engineered to have at least a portion of at least one domain (e.g., ECD, ICD, TMD, or C-terminal region) that is derived from sequences of one or more different transmembrane proteins or receptors.

The term “Site II interface,” “Site II region,” “Site II interface region,” or “Site II,” as used herein, refers to the larger of the G-CSF:G-CSFR 2:2 heterodimer binding interfaces of G-CSF with G-CSFR, located at the interface between G-CSF and the Cytokine Receptor Homologous (CRH) domain of G-CSFR.

The term “Site III interface,” “Site III region,” “Site III interface region,” or “Site III,” as used herein, refers to the smaller of the G-CSF:G-CSFR 2:2 heterodimer binding interfaces of G-CSF with G-CSFR, and is located at the interface between G-CSF and the N-terminal Ig-like domain of G-CSFR.

The term “at least a portion of” or “a portion of,” as used herein, in certain aspects refers to greater than 75%, greater than 80%, greater than 90%, greater than 95%, greater than 99% of the length of contiguous nucleic acid bases or amino acids of a SEQ ID NO described herein. In certain aspects, at least a portion of a domain or binding site (e.g., ECD, ICD, transmembrane, C-terminal region or signaling molecule binding site) described herein can be greater than 75%, greater than 80%, greater than 90%, greater than 95%, greater than 99% identical to a SEQ ID NO. described herein.

The term “wild-type” refers to the native amino acid sequence of a polypeptide or native nucleic acid sequence of a gene coding for a polypeptide described herein. The wild-type sequence of a protein or gene is the most common sequence of the polypeptide or gene for a species for that protein or gene.

The terms “variant cytokine-receptor pair,” “variant cytokine and receptor pairs,” “variant cytokine and receptor design(s),” “variant cytokine-receptor switch,” or “orthogonal cytokine-receptor pair” refers to genetically engineered pairs of proteins that are modified by amino acid changes to (a) lack binding to the native cytokine or cognate receptor; and (b) to specifically bind to the counterpart engineered (variant) ligand or receptor.

The term “variant receptor,” or “orthogonal receptor” as used herein, refers to the genetically engineered receptor of a variant cytokine-receptor pair and includes chimeric receptors.

The term “variant ECD,” as used herein, refers to the genetically engineered extracellular domain of a receptor (e.g., G-CSFR) of a variant cytokine-receptor pair.

The term “variant cytokine,” “variant G-CSF,” or “orthogonal cytokine” as used herein, refers to the genetically engineered cytokine of a variant cytokine-receptor pair.

As used herein, “do not bind,” “does not bind” or “incapable of binding” refers to no detectable binding, or an insignificant binding, i.e., having a binding affinity much lower than that of the natural ligand.

The term “selectively activates,” or “selective activation,” as used herein when referring to a cytokine and a variant receptor, refers to a cytokine that binds preferentially to a variant receptor and the receptor is activated upon binding of a cytokine to the variant receptor. In certain aspects, the cytokine selectively activates a chimeric receptor that has been co-evolved to specifically bind the cytokine. In certain aspects, the cytokine is a wild-type cytokine and it selectively activates a chimeric receptor that is expressed on cells, whereas the native, wild-type receptor to the cytokine is not expressed in the cells.

The term, “enhanced activity,” as used herein, refers to increased activity of a variant receptor expressed on a cell upon stimulation with a variant cytokine, wherein the activity is an activity observed for a native receptor upon stimulation with a native cytokine.

The term, “antigen binding signaling receptor” refers to any cell surface protein or protein complex that can bind an antigen and generate an intracellular signal upon binding to the antigen.

The term “agonistic signaling protein”, as used herein, refers to a protein that binds a target binding molecule (e.g., protein receptor or antigen), and the binding induces one or more signaling events in the target cell harboring the target binding molecule. Conversely, the term, “antagonistic signaling protein” refers to a protein that binds a target binding molecule (e.g., protein receptor or antigen), and the binding inhibits one or more signaling events in the target cell harboring the target binding molecule (by e.g., interfering with other agonistic proteins to bind the same target molecule).

The term “affinity reagent”, as used herein, refers to any molecule (e.g, protein, nucleic acid, etc.) that has an ability to bind any target molecule.

The term “immune cell” refers to any cell that is known to function to support the immune system of an organism (including innate and adaptive immune responses), and includes, but is not limited to, Lymphocytes (e.g., B cells, plasma cells and T cells), Natural Killer Cells (NK cells), Macrophages, Monocytes, Dendritic cells, Neutrophils, and Granulocytes. Immune cells include stem cells, immature immune cells and differentiated cells. Immune cells also include any sub-population of cells, however rare or abundant in an organism. In certain embodiments, an immune cell is identified as such by harboring known markers (e.g., cell surface markers) of immune cell types and sub-populations.

The term “T cells” refers to mammalian immune effector cells that may be characterized by expression of CD3 and/or T cell antigen receptor, which cells may be engineered to express an orthologous cytokine receptor. In some embodiments, the T cells are selected from naïve CD8⁺ T cells, cytotoxic CD8⁺ T cells, naïve CD4⁺ T cells, helper T cells, e. g., T_{H}2, T_{H}9, T_{H}11, T_{H}22, T_{FH}; regulatory T cells, e.g., TR1, natural T_{Reg}, inducible T_{Reg}; memory T cells, e.g., central memory T cells, effector memory T cells, NKT cells, and γδT cells.

The term “G-CSFR” refers to Granulocyte Colony-Stimulating Factor Receptor. G-CSFR can also be referred to as: GCSFR, G-CSF Receptor, Colony Stimulating Factor 3 Receptor, CSF3R, CD114 Antigen, or SCN7. Human G-CSFR is encoded by the gene having an Ensembl identification number of: ENSG00000119535. Human G-CSFR is encoded by the cDNA sequence corresponding to GeneBank Accession number NM_56039.3.

The term “G-CSF” refers to Granulocyte Colony Stimulating Factor. G-CSF can also be called Colony Stimulating Factor 3 and CSF3. Human G-CSF is encoded by the gene having an Ensembl identification number of: ENSG00000108342. Human G-CSF is encoded by the cDNA sequence corresponding to GeneBank Accession number KP271008.1.

“JAK” can also be referred to as Janus Kinase. JAK is a family of intracellular, nonreceptor tyrosine kinases that transduce cytokine-mediated signals via the JAK-STAT pathway and includes JAK1, JAK2, JAK3 and TYK2. Human JAK1 is encoded by the gene having an Ensembl identification number of: ENSG00000162434. Human JAK1 is encoded by the cDNA sequence corresponding to GeneBank Accession number NM_002227. Human JAK2 is encoded by the gene having an Ensembl identification number of: ENSG00000096968. Human JAK2 is encoded by the cDNA sequence corresponding to GeneBank Accession number_NM_001322194. Human JAK3 is encoded by the gene having an Ensembl identification number of: ENSG00000105639. Human JAK3 is encoded by the cDNA sequence corresponding to GeneBank Accession number NM_000215. Human_TYK2 is encoded by the gene having an Ensembl identification number of: ENSG00000105397. Human TYK2 is encoded by the cDNA sequence corresponding to GeneBank Accession number NM_001385197.

STAT can also be referred to as Signal Transducer and Activator of Transcription. STAT is a family of 7 STAT proteins: STAT1, STAT2, STAT3, STAT4, STAT5A, STAT5B and STAT6. Human STAT1 is encoded by the gene having an Ensembl identification number of: ENSG00000115415. Human STAT1 is encoded by the cDNA sequence corresponding to GeneBank Accession number NM_007315. Human_STAT2 is encoded by the gene having an Ensembl identification number of: ENSG00000170581. Human STAT2 is encoded by the cDNA sequence corresponding to GeneBank Accession number NM_005419. Human_STAT3 is encoded by the gene having an Ensembl identification number of: ENSG00000168610. Human STAT3 is encoded by the cDNA sequence corresponding to GeneBank Accession number NM_139276. Human STAT4 is encoded by the gene having an Ensembl identification number of: ENSG00000138378. Human STAT4 is encoded by the cDNA sequence corresponding to GeneBank Accession number NM_003151. Human STAT5A is encoded by the gene having an Ensembl identification number of: ENSG00000126561. Human STAT5A is encoded by the cDNA sequence corresponding to GeneBank Accession number NM_003152. Human STAT5B is encoded by the gene having an Ensembl identification number of: ENSG00000173757. Human STAT5B is encoded by the cDNA sequence corresponding to GeneBank Accession number_NM_012448. Human STAT6 is encoded by the gene having an Ensembl identification number of: ENSG00000166888. Human STAT6 is encoded by the cDNA sequence corresponding to GeneBank Accession number NM_003153.

SHC can also be referred to as Src Homology 2 Domain Containing Transforming Protein. Shc is a family of three isoforms and includes p66Shc, p52Shc and p46Shc, SHC1, SHC2 and SHC3. Human SHC1 is encoded by the gene having an Ensembl identification number of: ENSG00000160691. Human SHC1 is encoded by the cDNA sequence corresponding to GeneBank Accession number NM_183001. Human SHC2 is encoded by the gene having an Ensembl identification number of: ENSG00000129946. Human SHC2 is encoded by the cDNA sequence corresponding to GeneBank Accession number NM_012435. Human SHC3 is encoded by the gene having an Ensembl identification number of: ENSG00000148082. Human SHC3 is encoded by the cDNA sequence corresponding to GeneBank Accession number NM_016848.

SHP-2 can also be referred to as Protein Tyrosine Phosphaase Non-Receptor Type 11 (PTPN11) and Protein-Tyrosine Phosphatase 1D (PTP-1D). Human SHP-2 is encoded by the gene having an Ensembl identification number of: ENSG00000179295. Human SHP-2 is encoded by the cDNA sequence corresponding to GeneBank Accession number NM_001330437.

PI3K can also be referred to as Phosphatidylinositod-4,5-Bisphosphate 3-Kinase. The catalytic subunit of PI3K can be referred to as PIK3CA. Human PIK3CA is encoded by the gene having an Ensembl identification number of: ENSG00000121879. Human PIK3CA is encoded by the cDNA sequence corresponding to GeneBank Accession number NM_006218.

EPOR can also be referred to as Erythropoietin Receptor. Human EPOR is encoded by the gene having an Ensembl identification number of: ENSG00000187266. Human EPOR is encoded by the cDNA sequence corresponding to GeneBank Accession number NM_000121.

IFNγR1 can also be referred to as Interferon Gamma Receptor 1. Human IFNγR1 is encoded by the gene having an Ensembl identification number of: ENSG00000027697. Human IFNγR1 is encoded by the cDNA sequence corresponding to GeneBank Accession number NM_000416.

IFNγR2 can also be referred to as Interferon Gamma Receptor 2. Human IFNγR2 is encoded by the gene having an Ensembl identification number of: ENSG00000159128. Human IFNγR2 is encoded by the cDNA sequence corresponding to GeneBank Accession number NM_001329128.

IFNAR2 can also be referred to as Interferon Alpha and Beta Receptor Subunit 2 Human IFNAR2 is encoded by the gene having an Ensembl identification number of: ENSG00000159110. Human IFNAR2 is encoded by the cDNA sequence corresponding to GeneBank Accession number NM_000874.

Abbreviations used in this application include the following: ECD (extracellular domain), ICD (intracellular domain), TMD (transmembrane domain), a G-CSFR (Granulocyte-Colony Stimulating Factor Receptor), a G-CSF (Granulocyte-Colony Stimulating Factor), IL (Interleukin), IL-2R (Interleukin-2 receptor), IL-12R (Interleukin 12 Receptor), IL-21R (Interleukin-21 Receptor) and IL-7R or IL-7Rα (Interleukin-7 receptor), IL-18 (Interleukin-18), IL-21 (Interleukin-21), IL-17 (Interleukin-17), TNF-α (Tumor Necrosis Factor Alpha), CXCL13 (C-X-C Motif Chemokine Ligand 13), CCL3 (C-C Motif Chemokine Ligand 3 or MIP-1α), CCL4 (C-C Motif Chemokine Ligand 4 or MIP-1β), CD40 ligand, B cell activating factor (BAFF), Flt3 ligand, CCL21 (C-C Motif Chemokine Ligand 21), CCL5 (C-C Motif Chemokine Ligand 5), XCL1 (X-C Motif Chemokine Ligand 1), CCL19 (C-C Motif Chemokine Ligand 19), and NKG2D (Killer Cell Lectin Like Receptor K1). IL-2Rγ can also be referred to herein as: IL-2RG, IL-2Rgc, γc, or IL-2Rγ. For select chimeric cytokine receptor designs: “G-CSFRwt-ICD_{IL-2Rb}” is herein also referred to as “G/IL-2Rb”; “G-CSFRwt-ICDgc” is herein also referred to as “G/gc”; “G-CSFR137-ICDgp130-IL-2Rb” is herein also referred to as “G2R-2 with 137 ECD”; and “G-CSFR137-ICDIL-2Rb+GCSFR137-ICDgc” is herein also referred to as “G2R-1 with 137 ECD”.

It must be noted that, as used in the specification and the appended claims, the singular forms “a,” “an” and “the” include plural referents unless the context clearly dictates otherwise.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

### Variant Cytokine and Receptor Designs

Described herein are variant cytokine and receptor pairs for selective activation of the variant receptors. The variant receptors of the instant disclosure comprise an extracellular domain of G-CSFR; and the variant cytokine comprises a G-CSF (Granulocyte Colony-Stimulating Factor), which binds to and activates the variant receptor. In certain embodiments, the variant receptors are chimeric receptors comprising an ECD of G-CSFR and at least a portion of an ICD of a receptor different from G-CSFR.

### Variant G-CSF and Variant G-CSFR ECD Pairs

In certain aspects, the variant G-CSF and receptor designs described herein comprise at least one Site II interface region mutation, at least one Site III interface region mutation, and combinations thereof. In certain aspects, the variant G-CSF and receptor designs described herein comprise at least one Site II or Site III interface region mutation listed in Tables 2, 2A, 4 or 6.

In certain aspects, at least one mutation on the variant receptor in the site II interface region is located at an amino acid position of the G-CSFR extracellular domain selected from the group consisting of amino acid position: 141, 167, 168, 171, 172, 173, 174, 197, 199, 200, 202 and 288 of the G-CSFR extracellular domain (SEQ ID NO. 2).

In certain aspects, at least one mutation on the variant G-CSF site II interface region is located at an amino acid position of the G-CSF selected from the group consisting of amino acid position: 12, 16, 19, 20, 104, 108, 109, 112, 115, 116, 118, 119, 122 and 123 of G-CSF (SEQ ID NO. 1).

In certain aspects, at least one mutation on the variant receptor site II interface region is selected from the group of mutations of the G-CSFR extracellular domains consisting of: R141E, R167D, K168D, K168E, L171E, L172E, Y173K, Q174E, D197K, D197R, M199D, D200K, D200R, V202D, R288D, and R288E.

In certain aspects, at least one mutation on the variant G-CSF site II interface region is selected from the group of mutations of G-CSF consisting of: K16D, R, S12E, S12K, S12R, K16D, L18F, E19K, E19R, Q20E, D104K, D104R, L108K, L108R, D109R, D112R, D112K, T115E, T115K, T116D, Q119E, Q119R, E122K, E122R, and E123R.

In certain aspects, at least one mutation on the variant site III interface region is selected from the group of mutations of the G-CSFR extracellular domain selected for the group consisting of amino acid position: 30, 41, 73, 75, 79, 86, 87, 88, 89, 91, and 93 of SEQ ID NO. 2.

In certain aspects, at least one mutation on the variant site III interface region is selected from the group of mutations of the G-CSF selected for the group consisting of amino acid position: 38, 39, 40, 41, 46, 47, 48, 49, and 147 of SEQ ID NO. 1.

In certain aspects, at least one mutation on the variant receptor site III interface region is selected from the group of mutations of the G-CSFR extracellular domains consisting of S30D, R41E, Q73W, F75K, S79D, L86D, Q87D, 188E, L89A, Q91D, Q91K, and E93K.

In certain aspects, at least one mutation on the variant G-CSF site III interface region is selected from the group of mutations of G-CSF consisting of: T38R, Y39E, K40D, K40F, L41D, L41E, L41K, E46R, L47D, V48K, V48R, L49K, and R147E.

The variant cytokine and receptor pairs described herein can comprise mutations in either the site II region alone, the site III region alone or both the site II and site III regions.

The variant cytokine and receptor pairs described herein can have any number of site II and/or site III mutations described herein. In certain aspects, the variant G-CSF and receptors have the mutations listed in Table 4. In certain aspects, the variant receptor and/or variant G-CSF may have 1, 2, 3, 4, 5, 6, 7, 8, 9, 10 or more mutations described herein. In certain aspects, the variant cytokines described herein share at least 50%, at least 60%, at least 70%, at least 80%, at least 85%, at least 90%, at least 95%, at least 96%, at least 97%, at least 98%, or at least 99% amino acid identity to a variant cytokine described herein. In certain aspects, the variant cytokines described herein share at least 50%, at least 60%, at least 70%, at least 80%, at least 85%, at least 90%, at least 95%, at least 96%, at least 97%, at least 98%, or at least 99% amino acid identity to a variant cytokine of Table 21.

In certain aspects, the variant receptors described herein comprise G-CSFR ECD domains that share at least 50%, at least 60%, at least 70%, at least 80%, at least 85%, at least 90%, at least 95%, at least 96%, at least 97%, at least 98%, or at least 99% amino acid identity to a G-CSFR ECD SEQ ID NO. described herein. In certain aspects, the chimeric receptor comprises the ECD of G-CSFR having an amino acid sequence of SEQ ID NO. 2, 3, 6 or 8.

In certain aspects, the variant G-CSF described herein comprise an amino acid sequence that shares at least 50%, at least 60%, at least 70%, at least 80%, at least 85%, at least 90%, at least 95%, at least 96%, at least 97%, at least 98%, or at least 99% amino acid identity to a G-CSFR ECD SEQ ID NO. 1.

In certain aspects, the ECD of G-CSFR comprises at least one amino acid substitution selected from the group consisting of R41E, R141E, and R167D.

A variant cytokine and/or receptor may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders. In certain embodiments, the signal sequence is the signal sequence of G-CSFR or GM-CSFR. In certain embodiments, the signal sequence is SEQ ID NO: 11 or SEQ ID NO: 12.

In certain aspects, the variant receptor and/or variant G-CSF are modified, e.g., sugar groups, and polyethylene glycol (PEG), either naturally or synthetically to enhance stability. For example, in certain embodiments, variant cytokines are fused to the Fc domain of IgG, albumin, or other molecules to extend its half-life, e.g., by pegylation, glycosylation, and the like as known in the art. In certain embodiments, the variant cytokines described herein are modified by chemical pegylation. In certain embodiments, the variant G-CSF cytokines of Table 21 are pegylated. In certain embodiments, the variant G-CSF cytokines corresponding to SEQ ID NO: 83, 83-1, 83-2, 83-3, 83-4, 83-5 and 84 are modified by pegylation. In certain embodiments, the variant G-CSF cytokines and/or chimeric cytokine receptors described herein are modified by addition of PEG to the N-terminus and/or C-terminus of the protein. In certain embodiments, the variant G-CSF cytokines and/or chimeric cytokine receptors described herein are modified by addition of a compound comprising PEG. In certain embodiments, the PEG or PEG-containing compound is about 20 kDa or less. In certain embodiments, the PEG or PEG-containing compound is 20 kDa or less, 15 kDa or less, 10 kDa or less, 5 kDa or less, or 1 kDa or less.

Fc-fusion can also promote alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The variant cytokines can include the entire Fc region, or a smaller portion that retains the ability to extend the circulating half-life of a chimeric polypeptide of which it is a part. In addition, full-length or fragmented Fc regions can be variants of the wild-type molecule.

Upon binding of the variant cytokine to the variant receptor, the variant receptor activates signaling that is transduced through native cellular elements to provide for a biological activity that mimics that native response, but which is specific to a cell engineered to express the variant receptor. In certain aspects, the variant receptor and G-CSF pair do not bind their native, wild-type G-CSF or native, wild-type G-CSFR. Thus, in certain embodiments, the variant receptor does not bind to the endogenous counterpart cytokine, including the native counterpart of the variant cytokine, while the variant cytokine does not bind to any endogenous receptors, including the native counterpart of the variant receptor. In certain embodiments, the variant cytokine binds the native receptor with significantly reduced affinity compared to binding of the native cytokine to the native cytokine receptor. In certain embodiments, the affinity of the variant cytokine for the native receptor is less than 10×, less than 100×, less than 1,000× or less than 10,000× of the affinity of the native cytokine to the native cytokine receptor. In certain embodiments, the variant cytokine binds the native receptor with a K_{D} of greater than 1×10⁻⁴ M, 1×10⁻⁵ M, greater than 1×10⁻⁶ M; greater than 1×10⁻⁷ M, greater than 1×10⁻⁸ M, or greater than 1×10⁻⁹ M. In certain embodiments, the variant cytokine receptor binds the native cytokine with significantly reduced affinity compared to the binding of the native cytokine receptor to the native cytokine. In certain embodiments, the variant cytokine receptor binds the native cytokine less than 10×, less than 100×, less than 1,000× or less than 10,000× the native cytokine to the native cytokine receptor. In certain embodiments, the variant cytokine receptor binds the native cytokine with a K_{D} of greater than 1×10⁻⁴ M, 1×10⁻⁵ M, greater than 1×10⁻⁶ M; or greater than 1×10⁻⁷ M, greater than 1×10⁻⁸ M, or greater than 1×10⁻⁹ M. In some embodiments, the affinity of the variant cytokine for the variant receptor is comparable to the affinity of the native cytokine for the native receptor, e.g. having an affinity that is least about 1% of the native cytokine receptor pair affinity, at least about 5%, at least about 10%, at least about 25%, at least about 50%, at least about 75%, at least about 100%, and may be higher, e.g. 2×, 3×, 4×, 5×, 10× or more of the affinity of the native cytokine for the native receptor. The affinity can be determined by any number of assays well known to one of skill in the art. For example, affinity can be determined with competitive binding experiments that measure the binding of a receptor using a single concentration of labeled ligand in the presence of various concentrations of unlabeled ligand. Typically, the concentration of unlabeled ligand varies over at least six orders of magnitude. Through competitive binding experiments, IC₅₀ can be determined. As used herein, “IC₅₀” refers to the concentration of the unlabeled ligand that is required for 50% inhibition of the association between receptor and the labeled ligand. IC₅₀ is an indicator of the ligand-receptor binding affinity. Low IC₅₀ represents high affinity, while high IC₅₀ represents low affinity.

Binding of a variant cytokine to the variant cytokine receptor expressed on the surface of a cell, may or may not affect the function of the variant cytokine receptor (as compared to native cytokine receptor activity); native activity is not necessary or desired in all cases. In certain embodiments, the binding of a variant cytokine to the variant cytokine receptor will induce one or more aspects of native cytokine signaling. In certain embodiments, the binding of a variant cytokine to the variant cytokine receptor expressed on the surface of a cell causes a cellular response selected from the group consisting of proliferation, viability, persistence, cytotoxicity, cytokine secretion, memory, and enhanced activity.

TABLE 1
Sequence of human WT G-CSF and
human WT G-CSFR Ig-CRH domain

| | | | Poly- | SEQ |
|---|---|---|---|---|
| | Uniprot | Residue | peptide | ID |
| Protein | ID | numbering | Sequence | NO. |
| G-CSF | A0A0B4U | 1-174 | TPLGPASSLP | 1 |
| | 5E3 | | QSFLLKCLEQ | |
| | | | VRKIQGDGAA | |
| | | | LQEKLCATYK | |
| | | | LCHPEELVLL | |
| | | | GHSLGIPWAP | |
| | | | LSSCPSQALQ | |
| | | | LAGCLSQLHS | |
| | | | GLFLYQGLLQ | |
| | | | ALEGISPELG | |
| | | | PTLDTLQLDV | |
| | | | ADFATTIWQQ | |
| | | | MEELGMAPAL | |
| | | | QPTQGAMPAF | |
| | | | ASAFORRAGG | |
| | | | VLVASHLQSF | |
| | | | LEVSYRVLRH | |
| | | | LAQP | |
| Human | | | MTPLGPASSL | SEQ |
| G-CSF | | | PQSFLLKCLE | ID |
| recombinantly | | | QVRKIQGDGA | NO. |
| produced | | | ALQEKLCATY | 229) |
| in E. coli | | | KLCHPEELVL | |
| | | | LGHSLGIPWA | |
| | | | PLSSCPSQAL | |
| | | | QLAGCLSQLH | |
| | | | SGLFLYQGLL | |
| | | | QALEGISPEL | |
| | | | GPTLDTLQLD | |
| | | | VADFATTIWQ | |
| | | | QMEELGMAPA | |
| | | | LQPTQGAMPA | |
| | | | FASAFQRRAG | |
| | | | GVLVASHLQS | |
| | | | FLEVSYRVLR | |
| | | | HLAQP | |
| G- | Q99062 | Ig: | EECGHISVSA | 2 |
| CSFR | | 1-97 | PIVHLGDPIT | |
| (Ig-CRH) | | CRH: | ASCIIKQNCS | |
| | | 98-308 | HLDPEPQILW | |
| | | | RLGAELQPGG | |
| | | | RQQRLSDGTQ | |
| | | | ESIITLPHLN | |
| | | | HTQAFLSCSL | |
| | | | NWGNSLQILD | |
| | | | QVELRAGYPP | |
| | | | AIPHNLSCLM | |
| | | | NLTTSSLICQ | |
| | | | WEPGPETHLP | |
| | | | TSFTLKSFKS | |
| | | | RGNCQTQGDS | |
| | | | ILDCVPKDGQ | |
| | | | SHCSIPRKHL | |
| | | | LLYQNMGIWV | |
| | | | QAENALGTSM | |
| | | | SPQLCLDPMD | |
| | | | VVKLEPPMLR | |
| | | | TMDPSPEAAP | |
| | | | PQAGCLQLSW | |
| | | | EPWQPGLHIN | |
| | | | QKCELRHKPQ | |
| | | | RGEASWALVG | |
| | | | PLPLEALQYE | |
| | | | LCGLLPATAY | |
| | | | TLQIRCIRWP | |
| | | | LPGHWSDWSP | |
| | | | SLELRTTE | |

TABLE 1A
Sequence of human WT G-CSF and
human WT G-CSFR Ig-CRH domain

| | | Residue | | SEQ |
|---|---|---|---|---|
| | Uniprot | number- | Polypeptide | ID |
| Protein | ID | ing | Sequence | NO. |
| G- | Q99062 | Ig: | ECGHISVSAPIVHLGD | 101 |
| CSFR | | 2-97 | PITASCIIKQNCSHL | |
| (Ig- | | CRH: | DPEPQILWRLGAELQ | |
| CRH) | | 98-308 | PGGRQQRLSDGTQES | |
| | | | IITLPHLNHTQAFLS | |
| | | | CSLNWGNSLQILDQV | |
| | | | ELRAGYPPAIPHNLS | |
| | | | CLMNLTTSSLICQWE | |
| | | | PGPETHLPTSFTLKS | |
| | | | FKSRGNCQTQGDSIL | |
| | | | DCVPKDGQSHCSIPR | |
| | | | KHLLLYQNMGIWVQA | |
| | | | ENALGTSMSPQLCLD | |
| | | | PMDVVKLEPPMLRTM | |
| | | | DPSPEAAPPQAGCLQ | |
| | | | LSWEPWQPGLHINQK | |
| | | | CELRHKPQRGEASWA | |
| | | | LVGPLPLEALQYELC | |
| | | | GLLPATAYTLQIRCI | |
| | | | RWPLPGHWSDWSPSL | |
| | | | ELRTTE | |

The G-CSFR amino acid sequence set forth in SEQ ID NO. 2 of Table 1 corresponds to the G-CSFR amino acid positions for the mutation numbering used herein. The G-CSFR sequence set forth in SEQ ID NO. 101 listed in Table TA is identical to SEQ ID NO. 2, but for one amino acid (glutamic acid) at the N-terminus that is removed. Thus, the G-CSFR amino acid positions for the mutation numbering used herein corresponds to amino acids 2-308 of SEQ ID NO. 101.

TABLE 2
Site II designs with mutations for G-CSF_{E} and G-CSFR_{E}.

| | Mutations | Mutations |
|---|---|---|
| Design | G-CSF_{E} | G-CSFR_{E} |
| 1 | E19K | R288E |
| 2 | S12K_E19K | R288E |
| 6 | D104K | K168D |
| 7 | D104R_L108R_T115K | 168D_L171E_Q174E |
| 8 | S12R_L18F_D104R_L108K_D112R | R167D_K168D_D197E |
| 9 | D104R_L108K_D112R_Q119R_E122K | R141E_R167D_K168D_Q174D |
| 15 | K16D_T115E_T116D_Q119E | L171R_Y173V_D197R |
| 17 | E122R_E123R | R141E |
| 18 | E19K_Q20E | Y173K_R288E |
| 19 | K16E_E19K | D200R_R288E |
| 20 | E19K_D104K | K168D_R288E |
| 21 | E19K_D112R | L172E_R288E |
| 22 | E19K_D104K | K168E_M199D_R288D |
| 23 | S12E_K16D_E19K | D197K_D200K_R288E |
| 24 | E19R_D112K | R167D_V202D_R288E |
| 25 | E19K_D109R_D112R | R167D_M199D_R288D |
| 27 | E19R_D104R_L108K_D112R | R167D_K168D_V202E_I239V_R288E |
| 28 | E19K_D109K_D112R_E122R_E123R | R141E_R167D_M199D_R288E |
| 30 | L108K D112R | R167D |
| 34 | L108K_D112R_E122R_E123R | R141E_R167D |
| 35 | T115K_E122R_E123R | R141E_L171E_Q174E |
| 36 | S12E_K16E_T115E_Q119E | L171R_D197K_D200R |
| 42 | E19K_D109K_D112R | R167D_L172E_M199D_R288D |
| 43 | E19R_D104R_D112K | R167D_K168D_V202D_R288E |
| 44 | E19K_D104R_L108R_D112R | R167D_K168D_R288D |
| 45 | E19K_D112K_T115K | R167E_L171E_Q174E_R288E |
| 46 | E19K_D104K_E122R_E123R | R141E_K168D_R288E |
| 47 | E19R_E122R_E123R | R141E_V202E_I239V_R288E |
| 48 | E19K_D109K_D112R | R167F_L172E_M199D_R288D |
| 49 | K16E_E19R_T115E_Q119E | L171R_D197K_D200K_R288E |
| 50 | E19K_D104R_L108K_D112R | R167D_K168D_V202E_R288E |
| 80 | D112K T115K | R167E Q174E |
| 81 | E19K | R288D |
| 82 | L108K_D112R | R141E_R167D |
| 83 | E19R_D112K | R167D_R288E |

TABLE 3
Site III designs.

| | Mutations | Mutations |
|---|---|---|
| Design # | G-CSF_{E} | G-CSFR_{E} |
| 51 | E46R_L49K | S30D_R41E |
| 52 | K40D_L41D | F75K_Q91K |
| 53 | L41K_E46R | R41E_Q91D |
| 54 | L41E_L47D | I88K |
| 55 | T38R_E46R | R41E_Q73E |
| 56 | K40D_R147E | F75K_E93K |
| 57 | E46R_V48R | R41E_Q87E |
| 58 | E46R | R41E_L86D |
| 59 | K40D_E46R | R41E_F75K |
| 60 | E46R | R41E_I88D |
| 61 | E46R_L49F | R41E_L89A |
| 62 | L41D | Q91K |
| 63 | T38R | Q73E |
| 64 | Y39E_K40D | F75K |
| 65 | L41K | I88E_Q91D |
| 66 | E46R | R41E |
| 67 | E46R | R41E_S79D |
| 68 | K40D | F75K |
| 69 | V48K | Q87D |
| 70 | R147E | E93K |
| 71 | L41K | Q91D |
| 72 | K40F | F75K |

TABLE 4
Examples of designs resulting from combinations of site II and III designs.

| | | | Parental | Parental |
|---|---|---|---|---|
| Design | Mutations | Mutations | site II | site III |
| Number | G-CSF_{E} | G-CSFR_{E} | design | design |
| 106 | E46R_D104K | R41E_K168D | 6 | 66 |
| 117 | E46R_E122R_E123R | R41E_R141E | 17 | 66 |
| 130 | E46R_L108K_D112R | R41E_R167D | 30 | 66 |
| 134 | E46R_L108K_D112R_E122R_E123R | R41E_R141E_R167D | 34 | 66 |
| 135 | E46R_T115K_E122R_E123R | R41E_R141E_L171E_Q174E | 35 | 66 |
| 137 | E46R_L108K_D112R | R41E_R141E_R167D | 82 | 66 |
| 300 | K40D_L41D_L108K_D112R | F75K_Q91K_R167D | 30 | 52 |
| 301 | T38R_E46R_L108K_D112R | R41E_Q73E_R167D | 30 | 55 |
| 302 | E46R_L108K_D112R | R41E_L86D_R167D | 30 | 58 |
| 303 | L108K D112R_R147E | E93K_R167D | 30 | 70 |
| 304 | E46R_L108K_D112R_R147E | R41E_E93K_R167D | 30 | 66, 70 |
| 305 | E19K_E46R_L108K_D112R | R41E_R167D_R288E | 1, 30 | 66 |
| 307 | S12E_K16D_E19K_E46R | R41E_D197K_D200K_R288E | 23 | 66 |
| 308 | E19R_E46R_D112K | R41E_R167D_V202D_R288E | 24 | 66 |
| 400 | E19K_E46R_D109R_D112R | R41E_R167D_M199D_R288D | 25 | 66 |
| 401 | E19K_E46R_L108K_D112R | R41E_R167D_R288D | 30, 81 | 66 |
| 402 | E19K_E46R_D112K_T115K | R41E_R167E_Q174E_R288E | 1, 80 | 66 |
| 403 | E19R_E46R_D112K | R41E_R167D_R288E | 83 | 66 |

TABLE 4A
Exemplary G-CSFR and G-CSF pairs

| G-CSF | | Corresponding |
|---|---|---|
| Variant | G-CSF | G-CSFR |
| Number | Mutations | Mutations |
| 130 | E46R_L108K_D112R | R41E_R167D |
| 130a1 | E46R_L108K_D112R_E122K | R41E_R141E_R167D |
| 130b1 | E46R_L108K_D112R_E123K | |
| 130a2 | E46R_L108K_D112R_E122R | |
| 130b2 | E46R_L108K_D112R_E123R | |
| 130a1b1 | E46R_L108K_D112R_E122K_E123K | |
| 134 | E46R_L108K_D112R_E122R_E123R | |

### Chimeric Receptors

In certain aspects, the variant receptors described herein are chimeric receptors. A chimeric receptor can comprise any of the variant G-CSFR ECD domains described herein. In certain aspects, the chimeric receptor further comprises at least a portion of the intracellular domain (ICD) of a different cytokine receptor. The intracellular domain of the different cytokine receptor can be selected from the group consisting of: gp130 (glycoprotein 130, a subunit of the interleukin-6 receptor or IL-6R), IL-2Rβ3 or IL-2Rb (interleukin-2 receptor beta), IL-2Rγ or γc or IL-2RG (interleukin-2 receptor gamma), IL-7Rα (interleukin-7 receptor alpha), IL-12Rβ₂ (interleukin-12 receptor beta 2), IL-21R (interleukin-21 receptor), IL-4R (interleukin-4 receptor), EPOR (erythropoietin receptor), IFNAR (interferon alpha/beta receptor) or IFNAR (interferon gamma receptor). In certain aspects, at least a portion of the intracellular domain comprises an amino acid sequence that shares at least 70%, at least 80%, at least 85%, at least 90%, at least 95%, at least 96%, at least 97%, at least 98%, or at least 99% amino acid identity to the amino acid sequence of a cytokine receptor ICD described herein. In certain aspects, at least a portion of a cytokine receptor ICD shares at least 70%, at least 80%, at least 85%, at least 90%, at least 95%, at least 96%, at least 97%, at least 98%, or at least 99% amino acid sequence identity to SEQ ID NO. 4, 7 or 9. In certain aspects, at least a portion of the intracellular domain comprises an amino acid sequence that shares at least 70%, at least 80%, at least 85%, at least 90%, at least 95%, at least 96%, at least 97%, at least 98%, or at least 99% amino acid identity to the amino acid sequence of a cytokine receptor ICD of a cytokine receptor listed in Table 26.

In certain embodiments, described herein are chimeric cytokine receptors comprising an extracellular domain (ECD) of a G-CSFR (Granulocyte-Colony Stimulating Factor Receptor) operatively linked to a second domain; the second domain comprising at least a portion of an intracellular domain (ICD) of a multi-subunit cytokine receptor, e.g., IL-2R. In certain aspects, the chimeric cytokine receptor comprises a portion of an ICD from Table 15A, Table 15B, Table 23 and Table 24. In certain aspects, the chimeric cytokine receptor comprises a transmembrane domain selected from Table 15A and Table 15B. In certain aspects, the chimeric cytokine receptor ICD comprises Box1 and Box 2 regions from Table 15A, Table 15B, Table 16 Table 23 and Table 24.

In certain aspects, the chimeric cytokine receptor comprises at least one signaling molecule binding site from Table 15A, Table 15B, Table 16, Table 23, Table 24 and Table 27. In some embodiments, the signaling molecule binding site(s) of the ICD comprise a sequence set forth in SEQ ID NO. 118-146. In some embodiments, the signaling molecule binding site(s) of the ICD comprise a sequence at least 80%, at least 90%, at least 91%, at least 92%, at least 93%, at least 94%, at least 95%, at least 96%, at least 97%, at least 98%, at least 99% or 100% identical to a sequence set forth in at least one of SEQ ID NO. 118-146.

In certain aspects, the chimeric receptors described herein comprise amino acid sequences in N-terminal to C-terminal order of the sequences disclosed in each of Tables 17-20, 23, 24, 26 and 32. In certain aspects, the sequences of the chimeric receptors described herein comprise nucleic acid sequences in 5′ to 3′ order of the sequences disclosed in each of Tables 17-20. In certain aspects, the chimeric cytokine receptor shares at least 50%, at least 60%, at least 70%, at least 80%, at least 85%, at least 90%, at least 95%, at least 96%, at least 97%, at least 98%, or at least 99% amino acid identity to the amino acid sequences in N-terminal to C-terminal order of the amino acid sequences disclosed in each of Tables 17-20 and 23, 24, 26 and 32. In certain aspects, the chimeric cytokine receptor shares at least 50%, at least 60%, at least 70%, at least 80%, at least 85%, at least 90%, at least 95%, at least 96%, at least 97%, at least 98%, or at least 99% nucleic acid identity to the nucleic acid sequences in 5′ to 3′ order of the nucleic acid sequences disclosed in each of Tables 17-20.

In certain aspects, the chimeric receptors described herein comprise at least a portion of an ICD of a cytokine receptor that shares at least 80%, at least 85%, at least 90%, at least 95%, at least 96%, at least 97%, at least 98%, or at least 99% amino acid or nucleic acid sequence identity to an ICD SEQ ID NO. described herein. In certain aspects, the chimeric receptor comprises at least a portion of an ICD of IL-2Rβ having an amino acid sequence of SEQ ID NO. 26, 29, 31, 39, 41, 43, 45, 47, or 49. In certain aspects, the chimeric receptor comprises at least a portion of an ICD of IL-2Rβ, i.e., IL-2Rb, having a nucleic acid sequence of SEQ ID NO. 54, 57, 59, 67, 69, 71, 73, 75, or 77. In certain aspects, the chimeric receptor comprises at least a portion of an ICD of IL-7Rα having an amino acid sequence of SEQ ID NO. 51 or a nucleic acid sequence of SEQ ID NO. 79. In certain aspects, the chimeric receptor comprises at least a portion of an ICD of IL-7R having an amino acid sequence of SEQ ID NO. 53 or a nucleic acid sequence of SEQ ID NO. 81. In certain aspects, the chimeric receptor comprises at least a portion of an ICD of IL-21R having an amino acid sequence of SEQ ID NO. 35 or 37. In certain aspects, the chimeric receptor comprises at least a portion of an ICD of IL-21R having a nucleic acid sequence of SEQ ID NO. 63 or 65 In certain aspects, the chimeric receptor comprises at least a portion of an ICD of IL-12Rβ₂ having an amino acid sequence of SEQ ID NO. 33, 42, or 46. In certain aspects, the chimeric receptor comprises at least a portion of an ICD of IL-12Rβ₂ having a nucleic acid sequence of SEQ ID NO. 61, 70 or 74. In certain aspects, the chimeric receptor comprises at least a portion of an ICD of G-CSFR having an amino acid sequence of SEQ ID NO. 30, 32, 34, 36, 38, 40, 44, 50 or 52. In certain aspects, the chimeric receptor comprises at least a portion of an ICD of G-CSFR having a nucleic acid sequence of SEQ ID NO. 58, 60, 62, 64, 66, 68, 72, 78 or 80. In certain aspects, the chimeric receptor comprises at least a portion of an ICD of gp130 having an amino acid sequence of SEQ ID NO. 28 or 48. In certain aspects, the chimeric receptor comprises at least a portion of an ICD of gp130 having a nucleic acid sequence of SEQ ID NO. 56 or 76. In certain aspects, the chimeric receptor comprises at least a portion of an ICD of IL-2Rγ (i.e., IL-2RG, IL-2Rgc, γc, or IL-2Rγ) having an amino acid sequence of SEQ ID NO. 27. In certain aspects, the chimeric receptor comprises at least a portion of an ICD of IL-2Rγ (i.e., IL-2RG, IL-2Rgc, γc, or IL-2Rγ) having a nucleic acid sequence of SEQ ID NO. 55.

In certain aspects, at least a portion of the ICDs described herein comprise at least one signaling molecule binding site. In certain aspects, at least one signaling molecule binding site is a STAT3 binding site of G-CSFR; a STAT3 binding site of gp130; a SHP-2 binding site of gp130; a Shc binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT5 binding site of IL-12Rβ₂; a STAT4 binding site of IL-12Rβ₂; a STAT3 binding site of IL-12Rβ₂; a STAT5 binding site of IL-21R; a STAT3 binding site of IL-21R; and a STAT1 binding site of IL-21R. In certain aspects, at least one signaling molecule binding site comprises a sequence that further comprises an amino acid listed in Table 16.

In certain embodiments, the chimeric receptor comprises at least one signaling molecule binding site from an intracellular domain of a cytokine receptor. In certain embodiments, the chimeric receptor comprises at least one signaling molecule binding site from an intracellular domain in Table 24. In certain embodiments, the at least one signaling molecule binding site is selected from the group consisting of: a SHC binding site of Interleukin (IL)-2Rβ; a STAT5 binding site of IL-2Rβ, an IRS-1 or IRS-2 binding site of IL-4Rα, a STAT6 binding site of IL-4Rα, a SHP-2 binding site of gp130, a STAT3 binding site of gp130, a SHP-1 or SHP-2 binding site of Erythropoietin Receptor (EPOR), a STAT5 binding site of EPOR, a STAT1 or STAT2 binding site of Interferon Alpha and Beta Receptor Subunit 2 (IFNAR2), and a STAT1 binding site of Interferon Gamma Receptor 1 (IFNγR1), or combinations thereof.

In certain embodiments, the chimeric receptor ICD further comprises at least one Box 1 region and at least one Box 2 region of at least one protein selected from the group consisting of G-CSFR, gp130, EPOR, and Interferon Gamma Receptor 2 (IFNγR2), or combinations thereof.

In certain aspects, at least a portion of the ICDs described herein comprise the Box 1 and Box regions of gp130 or G-CSFR. In certain aspects the Box 1 region comprises a sequence of amino acids listed in Table 2. In certain aspects the Box 1 region comprises an amino acid sequence that is greater than 50% identical to a Box 1 sequence listed in Table 16.

In certain aspects, the ICD comprises: (a) an amino acid sequence of one or both of SEQ ID NO. 90 or 91; or (b) an amino acid sequence of one or both of SEQ ID NO. 90 or 92; or (c) an amino acid sequence of SEQ ID NO. 93; or (d) an amino acid sequence of SEQ ID NO. 94; or (e) an amino acid sequence of one or both of SEQ ID NO. 95 or 96; or (f) an amino acid sequence of SEQ ID NO. 97 or 98; or (g) an amino acid sequence of SEQ ID NO. 99 or 100.

In certain aspects, the intracellular domain of the different cytokine receptor is a wild-type intracellular domain.

In certain aspects, the chimeric variant receptors described herein further comprise at least a portion of the transmembrane domain (TMD) of G-CSFR. In certain aspects, the chimeric variant receptors described herein further comprise at least a portion of the transmembrane domain (TMD) of a different cytokine receptor. The TMD of the different cytokine receptor can be selected from the group consisting of: G-CSFR, gp130 (glycoprotein 130), IL-2Rβ (interleukin-2 receptor beta), IL-2Rγ or γc (IL-2 receptor gamma), IL-7Rα (interleukin-7 receptor alpha), IL-12Rβ₂ (interleukin-12 receptor beta 2) and IL-21R (interleukin-21 receptor). In certain aspects, at least a portion of the TMD comprises an amino acid sequence that shares at least 70%, at least 80%, at least 85%, at least 90%, at least 95%, at least 96%, at least 97%, at least 98%, or at least 99% amino acid identity to the amino acid sequence of a cytokine receptor TMD described herein. In certain aspects, at least a portion of a cytokine receptor TMD shares at least 70%, at least 80%, at least 85%, at least 90%, at least 95%, at least 96%, at least 97%, at least 98%, or at least 99% amino acid sequence identity to SEQ ID NO. 4, 5, 7 or 9.

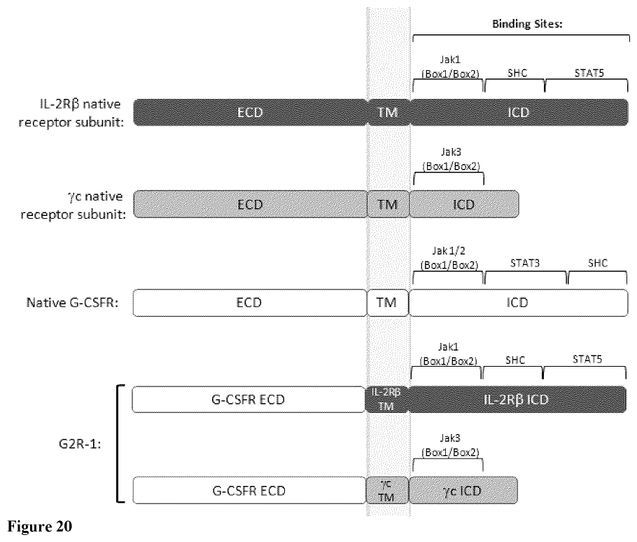

In certain aspects, the chimeric receptors described herein comprise a G-CSFR ECD domain, a transmembrane domain (TMD), and at least one portion of one ICD arranged in N-terminal to C-terminal order, as shown in the chimeric receptor designs of FIGS. 20, 23 and 24.

In certain aspects, the chimeric receptor comprises the G-CSFR ECD of SEQ ID NO. 3, a portion of the gp130 TMD and ICD of SEQ ID NO 4, and a portion of the IL-2Rβ ICD of SEQ ID NO. 5. In certain aspects, the chimeric receptor comprises the G-CSFR ECD of SEQ ID NO. 6, and a portion of the IL-2Rβ ICD of SEQ ID NO. 7. In certain aspects, the chimeric receptor comprises the G-CSFR ECD of SEQ ID NO. 8, and a portion of the IL-2Rγ ICD of SEQ ID NO. 9.

Binding of a variant or wild type cytokine to the chimeric cytokine receptor expressed on the surface of a cell, may or may not affect the function of the variant cytokine receptor (as compared to native cytokine receptor activity); native activity is not necessary or desired in all cases. In certain embodiments, the binding of a variant cytokine to the chimeric cytokine receptor will induce one or more aspects of native cytokine signaling. In certain embodiments, the binding of a variant cytokine to the chimeric cytokine receptor expressed on the surface of a cell causes a cellular response selected from the group consisting of proliferation, viability, persistence, cytotoxicity, cytokine secretion, memory, and enhanced activity.

### Receptors Comprising an ECD of IL-7Rα

In certain aspects, disclosed herein are chimeric receptors, comprising: (i) an extracellular domain (ECD) of Interleukin-7 Receptor alpha (IL-7Rα); (ii) a transmembrane domain (TMD); and (iii) an intracellular domain (ICD) of a cytokine receptor that is distinct from a wild-type, human IL-7Rα intracellular signaling domain set forth in SEQ ID NO:109; wherein the ECD and TMD are each operatively linked to the ICD. In some embodiments, the carboxy terminus (C-terminus) of the ECD is linked to the amino terminus (N-terminus) of the TMD, and the C-terminus of TMD is linked to the N-terminus of the ICD. In some embodiments, the ECD is the ECD of native human IL-7Rα. In some embodiments, the TMD is the TMD of IL-7Rα. In some embodiments, the TMD is the TMD of native human IL-7Rα. In some embodiments, the ICD comprises at least one signaling molecule binding site from an intracellular domain of a cytokine receptor, and, optionally, the at least one signaling molecule binding site comprises: (a) a JAK1 binding site (Box 1 and 2 region) of IL-2Rβ, IL-4Rα, IL-7Rα, IL-21R, or gp130; (b) a SHC binding site of IL-2Rβ; (c) a STAT5 binding site of IL-2Rβ or IL-7Rα; (d) a STAT3 binding site of IL-21R or gp130; (e) a STAT4 binding site of IL-12Rβ2; (f) a STAT6 binding site of IL-4Rα; (g) an IRS-1 or IRS-2 binding site of IL-4Rα; (h) a SHP-2 binding site of gp130; (i) a PI3K binding site of IL-7Rα; or combinations thereof. In some embodiments, the ICD comprises at least an intracellular signaling domain of a receptor that is activated by heterodimerization with the common gamma chain (γc) when the ICD is part of its native receptor. In some embodiments, the ICD of the chimeric receptor does not heterodimerize with the common gamma chain. In some embodiments, the ICD of the chimeric receptor homodimerizes upon activation. In some embodiments, the ICD comprises at least an intracellular signaling domain of a cytokine receptor selected from the group consisting of: IL-2Rβ (Interleukin-2 receptor beta), IL-4Rα (Interleukin-4 Receptor alpha), IL-9Rα (Interleukin-9 Receptor alpha), IL-12Rβ2 (Interleukin-12 Receptor), IL-21R (Interleukin-21 Receptor) and glycoprotein 130 (gp130), and combinations thereof.

In certain aspects, described herein are chimeric receptors, comprising an ECD of IL-7Rα and a TMD operatively linked to an ICD, the ICD comprising:
(i)
   (a) a Box 1 and a Box 2 region of IL-2Rβ;
   (b) a SHC binding site of IL-2Rβ; and
   (c) a STAT5 binding site of IL-2Rβ; or
(ii)
   (a) a Box 1 and a Box 2 region of IL-7Rα;
   (b) a SHC binding site of IL-2Rβ; and
   (c) a STAT5 binding site of IL-2Rβ; or
(iii)
   (a) a Box 1 and a Box 2 region of IL-2Rβ;
   (b) a SHC binding site of IL-2Rβ;
   (c) a STAT5 binding site of IL-2Rβ; and
   (d) a STAT4 binding site of IL-12Rβ2; or
(iv)
   (a) a Box 1 and a Box 2 region of IL-7Rα;
   (b) a SHC binding site of IL-2Rβ;
   (c) a STAT5 binding site of IL-2Rβ; and
   (d) a STAT4 binding site of IL-12Rβ2; or
(v)
   (a) a Box 1 and a Box 2 region of IL-21R; and
   (b) a STAT3 binding site of IL-21R; or
(vi)
   (a) a Box 1 and a Box 2 region of IL-7Rα; and
   (b) a STAT3 binding site of IL-21R; or
(vii)
   (a) a Box 1 and a Box 2 region of IL-21R;
   (b) a STAT3 binding site of IL-21R; and
   (c) a STAT5 and PI3 kinase binding site of IL-7Rα;
(viii)
   (a) a Box 1 and a Box 2 region of IL-7Rα;
   (b) a STAT3 binding site of IL-21R; and
   (c) a STAT5 and PI3 kinase binding site of IL-7Rα;
(ix)
   (a) a Box 1 and a Box 2 region of IL-2Rβ;
   (b) a SHC binding site of IL-2Rβ;
   (c) a STAT5 binding site of IL-2Rβ; and
   (d) a STAT3 binding site of IL-21R; or
(x)
   (a) a Box 1 and a Box 2 region of IL-7Rα;
   (b) a SHC binding site of IL-2Rβ;
   (c) a STAT5 binding site of IL-2Rβ; and
   (d) a STAT3 binding site of IL-21R; or
(xi)
   (a) a Box 1 and a Box 2 region of IL-2Rβ;
   (b) a SHC binding site of IL-2Rβ;
   (c) a STAT5 binding site of IL-2Rβ;
   (d) a STAT4 binding site of IL12Rβ2; and
   (e) a STAT3 binding site of IL-21R; or
(xii)
   (a) a Box 1 and a Box 2 region of IL-7Rα;
   (b) a SHC binding site of IL-2Rβ;
   (c) a STAT5 binding site of IL-2Rβ;
   (d) a STAT4 binding site of IL12Rβ2; and
   (e) a STAT3 binding site of IL-21R; or
(xiii)
   (a) a Box 1 and a Box 2 region of IL-4Rα;
   (b) an IRS-1 or IRS-2 binding site of IL-4Rα; and
   (c) a STAT6 binding site of IL-4Rα; or
(xiv)
   (a) a Box 1 and a Box 2 region of IL-7Rα;
   (b) an IRS-1 or IRS-2 binding site of IL-4α; and
   (c) a STAT6 binding site of IL-4α; or
(xv)
   (a) a Box 1 and a Box 2 region of gp130;
   (b) a SHP-2 binding site of gp130; and
   (c) a STAT3 binding site of gp130; or
(xvi)
   (a) a Box 1 and a Box 2 region of IL-7Rα;
   (b) a SHP-2 binding site of gp130; and
   (c) a STAT3 binding site of gp130.
      In some embodiments, (b) is N-terminal to (c); or (c) is N-terminal to (b); or (c) is N-terminal to (d); or (d) is N-terminal to (c); or (d) is N-terminal to (e); or (e) is N-terminal to (d).

In some embodiments, the ICDs described herein comprise the Box 1 and Box 2 regions of gp130 or G-CSFR. In certain aspects the Box 1 region comprises a sequence of amino acids listed in Table 16. In certain aspects the Box 1 region comprises an amino acid sequence that is greater than 50% identical to a Box 1 sequence listed in Table 16.

In some embodiments, the ICD comprises a sequence at least 80%, at least 90%, at least 91%, at least 92%, at least 93%, at least 94%, at least 95%, at least 96%, at least 97%, at least 98%, at least 99% or 100% identical to a sequence set forth in at least one of SEQ ID NO: 192-214.

In some embodiments, the signaling molecule binding site(s) of the ICD comprise a sequence set forth in SEQ ID NO. 118-146. In some embodiments, the signaling molecule binding site(s) of the ICD comprise a sequence at least 80%, at least 90%, at least 91%, at least 92%, at least 93%, at least 94%, at least 95%, at least 96%, at least 97%, at least 98%, at least 99% or 100% identical to a sequence set forth in at least one of SEQ ID NO. 118-146. In certain aspects, the chimeric cytokine receptor shares at least 50%, at least 60%, at least 70%, at least 80%, at least 85%, at least 90%, at least 95%, at least 96%, at least 97%, at least 98%, or at least 99% amino acid identity to the amino acid sequences in N-terminal to C-terminal order of the amino acid sequences disclosed in Table 32.

### Systems Comprising Chimeric Receptors and IL-7

In certain aspects, the present disclosure describes a system for selective activation of a receptor expressed on a cell surface, the system comprising: (a) a chimeric receptor described herein; and (b) IL-7.

In certain aspects, the present disclosure describes a system for selective activation of an immune cell, the system comprising: (a) a chimeric receptor described herein; (b) IL-7; and (c) an antigen binding signaling receptor.

In certain aspects, the present disclosure describes a system for selective activation of an immune cell, the system comprising: (a) a chimeric receptor described herein; (b) IL-7; and (c) at least one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the cytokine or chemokine is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, NKG2D, and combinations thereof. In some embodiments, the cytokine is IL-18. In some embodiments, the cytokine is human.

In some embodiments, the system further comprises at least one antigen binding signaling receptor. In some embodiments, the at least one antigen binding signaling receptor comprises at least one receptor selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In some embodiments, the at least one antigen binding signaling receptor is a CAR.

### Agonistic or Antagonistic Signaling Proteins

In certain aspects, the systems and method described herein comprise at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor(s). In some embodiments, the at least one additional cytokine(s) or chemokine(s) comprises at least one of interleukin (IL)-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, CCL19, and the receptor NKG2D, and combinations thereof.

### Antigen Binding Signaling Receptors

In certain aspects, the systems and method described herein comprise an antigen binding signaling receptor(s). In some embodiments, the antigen binding signaling receptor(s) comprises at least one of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. Any CAR known in the art can be used, including but not limited to, a mesothelin CAR, or any CAR described herein. Any TCR known in the art can be used, including but not limited to, a any TCR described herein.

### Nucleic Acids Encoding Variant Cytokines and Receptors

Included in this disclosure are nucleic acids encoding any one of the receptors and variant G-CSF described herein.

A variant receptor or variant G-CSF may be produced recombinantly not only directly, but also as a fusion polypeptide with a heterologous polypeptide, e.g., a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression, the native signal sequence may be used, or other mammalian signal sequences may be suitable, such as signal sequences from secreted polypeptides of the same or related species, as well as viral secretory leaders. In certain aspects, the signal sequence can be an amino acid sequence comprising the signal sequence at the N-terminal region of SEQ ID NO. 2, 3, 6 or 8. In certain aspects, the signal sequence can be the amino acid sequence of MARLGNCSLTWAALIILLLPGSLE (SEQ ID NO. 11).

Included in this disclosure are nucleic acids encoding any one of the receptors, IL-7, and variant G-CSF described herein.

A variant receptor, or wildtype or variant IL-7, or variant G-CSF may be produced recombinantly not only directly, but also as a fusion polypeptide with a heterologous polypeptide, e.g., a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression, the native signal sequence may be used, or other mammalian signal sequences may be suitable, such as signal sequences from secreted polypeptides of the same or related species, as well as viral secretory leaders. In certain aspects, the signal sequence can be an amino acid sequence comprising the signal sequence at the N-terminal region of SEQ ID NO. 2, 3, 6 or 8. In certain aspects, the signal sequence can be the amino acid sequence of MTILGTTFGMVFSLLQVVSG (SEQ ID NO. 190). In certain aspects, the signal sequence can be the amino acid sequence of MARLGNCSLTWAALIILLLPGSLE (SEQ ID NO. 11).

### Expression Vectors Encoding Variant Cytokines or Receptors

Described herein are also expression vectors, and kits of expression vectors, which comprise one or more nucleic acid sequence(s) encoding one or more of the variant receptors, variant G-CSF, or wild type or variant IL-7 described herein.

In certain embodiments, the nucleic acid encoding a variant receptor or variant G-CSF is inserted into a replicable vector for expression. Such a vector may be used to introduce the nucleic acid sequence(s) into a host cell so that it expresses a variant receptor or cytokine described herein. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like. The vector may, for example, be a plasmid or a viral vector, such as a retroviral vector, adenoviral vector, lentiviral vector, or a transposon-based vector or synthetic mRNA. The vector may be capable of transfecting or transducing a cell (e.g., a T cell, an NK cell or other cells).

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

In certain aspects, expression vectors contain a promoter that is recognized by the host organism and is operably linked to a variant protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are known from mammalian genes (globin, elastase, albumin, fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In certain aspects, disclosed herein are lentiviral vectors encoding the chimeric receptors disclosed herein. In certain aspects, the lentiviral vector comprises the HIV-1 5′ LT and a 3′ LTR. In certain aspects, the lentiviral vector comprises an EF1a promoter. In certain aspects, the lentiviral vector comprises an SV40 poly a terminator sequence. In certain aspects, the vector is psPAX2, Addgene® 12260, pCMV-VSV-G, or Addgene® 8454.

In some embodiments, also described are nucleic acid and polypeptide sequence with high sequence identity, e.g., 95, 96, 97, 98, 99% or more sequence identity to sequences described herein. The term percent sequence “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### Cells Expressing Variant Receptors and Variant Cytokines

Also described herein are cells expressing the variant receptors. Host cells, including engineered immune cells, can be transfected or transduced with the above-described expression vectors for variant cytokine or receptor expression.

In certain embodiments, the present disclosure provides a cell which comprises one or more of a variant receptor or variant cytokine described herein. The cell may comprise a nucleic acid or a vector encoding a variant receptor or variant cytokine described herein. This disclosure also provides methods of producing cells expressing a variant receptor. In certain aspects, the cells are produced by introducing into a cell the nucleic acid or expression vector described herein. The nucleic acid or expression vector can be introduced into the cell by any process including, but not limited to, transfection, transduction of a viral vector, transposition or gene editing. Any gene editing technique known in the art may be used including, but not limited to, techniques comprising clustered regularly interspaced short palindromic repeats (CRISPR-Cas) systems, zinc finger nucleases, transcription activator-like effector-based nucleases and meganucleases.

The host cell can be any cell in the body. In certain embodiments, the cell is an immune cell. In some embodiments, the cell is a T cell, including, but not limited to, naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., TR1, natural T_{Reg}, inducible T_{Reg}; memory T cells, e.g., central memory T cells, effector memory T cells, NKT cells, γδT cells; etc. In certain embodiments, the cell is a B cell, including, but not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc. In certain embodiments, the cell is an innate lymphoid cell, including, but not limited to, NK cells, etc. In certain embodiments, the cell is a myeloid cell, including, but not limited to, macrophages, dendritic cells, myeloid-derived suppressor cells, etc.

In certain embodiments, the cell is a stem cell, including, but not limited to, hematopoietic stem cells, mesenchymal stem cells, neural stem cells, etc.

In some embodiments, the cell is genetically modified in an ex vivo procedure, prior to transfer into a subject. The cell can be provided in a unit dose for therapy, and can be allogeneic, autologous, etc. with respect to an intended recipient.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarized below.

Helper T helper cells (Th cells) assist other white blood cells in immunologic processes, including maturation of B cells into plasma cells and memory B cells, and activation of cytotoxic T cells and macrophages. Th cells express CD4 on their surface. Th cells become activated when they are presented with peptide antigens by MHC class II molecules on the surface of antigen presenting cells (APCs). These cells can differentiate into one of several subtypes, including Th1, Th2, Th3, Th17, Th9, or Tfh, which secrete different cytokines to facilitate different types of immune responses.

Cytolytic T cells (TC cells, or CTLs) destroy virally infected cells and tumor cells, and are also implicated in transplant rejection. Most CTLs express the CD8 at their surface. These cells recognize their targets by binding to antigen associated with MHC class I, which is present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus. Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Treg cells can be distinguished from other T cells by the presence of an intracellular molecule called FoxP3.

Adaptive Treg cells (also known as Tr1 cells or Th3 cells) may originate during a normal immune response. The cell may be a Natural Killer cell (or NK cell). NK cells form part of the innate immune system. NK cells provide rapid responses to innate signals from virally infected cells in an MHC independent manner.

In certain aspects, the cells expressing the variant receptors or variant cytokines described herein are tumor-infiltrating lymphocytes (TILs) or tumor-associated lymphocytes (TALs). In certain aspects, the TILs or TALs comprise CD4+ T cells, CD8+ T cells, Natural Killer (NK) cells, and combinations thereof.

In certain embodiments, the T cells described herein are chimeric antigen receptor T cells (CAR-T cells) that have been genetically engineered to produce an artificial T-cell receptor for use in immunotherapy. In certain aspects, the CAR-T cells derived from T cells in a patient's own blood (i.e., autologous). In certain aspects, the CAR-T are derived from the T cells of another healthy donor (i.e., allogeneic). In certain aspects, the CAR-T cells are derived or synthesized from non-immune cell types such as pluripotent stem cells.

In certain embodiments, the T cells described herein are engineered T Cell Receptor (eTCR-T cells) that have been genetically engineered to produce a particular T Cell Receptor for use in immunotherapy. In certain aspects, the eTCR-T cells are derived from T cells in a patient's own blood (i.e., autologous). In certain aspects, the eTCR-T cells are derived from the T cells of a donor (i.e., allogeneic). In certain aspects, the eTCR-T cells are derived or synthesized from non-immune cell types such as pluripotent stem cells.

In certain aspects, the cells expressing chimeric cytokine receptors described herein are NK cells. NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation. In certain aspects, the NK cells are derived from NK cells in a patient's own blood (i.e., autologous). In certain aspects, the NK cells are derived from the NK cells of a donor (i.e., allogeneic). In certain aspects, the NK cells are derived or synthesized from non-immune cell types such as pluripotent stem cells.

In certain aspects, the cells expressing chimeric cytokine receptors described herein are B cells. B cells include, but are not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc. In certain aspects, the B cells are derived from B cells in a patient's own blood (i.e., autologous). In certain aspects, the B cells are derived from the B cells of a donor (i.e., allogeneic). In certain aspects, the B cells are derived or synthesized from non-immune cell types such as pluripotent stem cells.

In certain aspects, the cells expressing chimeric cytokine receptors described herein are myeloid cells, including, but not limited to, macrophages, dendritic cells, myeloid-derived suppressor cells, etc. In certain aspects, the myeloid cells are derived from myeloid cells in a patient's own blood (i.e., autologous). In certain aspects, the myeloid cells are derived from the myeloid cells of a donor (i.e., allogeneic). In certain aspects, the myeloid cells are derived or synthesized from non-immune cell types such as pluripotent stem cells.

The cells expressing a variant receptor or variant cytokine described herein may be of any cell type. In certain aspects, the cells expressing a variant receptor or variant cytokine described herein is a cell of the hematopoietic system. Immune cells (e.g., T cells or NK cells) according to the invention may either be created ex vivo either from a patient's own peripheral blood (1st party), or in the setting of a hematopoietic stem cell transplant from donor peripheral blood (2nd party), or peripheral blood from an unconnected donor (3rd party). Alternatively, immune cells described herein may be derived from ex vivo differentiation of inducible progenitor cells or embryonic progenitor cells to immune cells. Alternatively, an immortalized immune cell line which retains its effector function (e.g., a T-cell or NK-cell line that retains its lytic function; a plasma cell line that retains its antibody producing function, or a dendritic cell line or macrophage that retains its phagocytic and antigen presentation function) and could act as a therapeutic may be used. In all these embodiments, variant receptor-expressing cells are generated by introducing DNA or RNA coding for each variant receptor(s) by one of many means including transduction with a viral vector or transfection with DNA or RNA.

The cells described herein can be immune cells derived from a subject engineered ex vivo to express a variant receptor and/or variant cytokine. The immune cell may be from a peripheral blood mononuclear cell (PBMC) sample or a tumor sample. Immune cells may be activated and/or expanded prior to being transduced with nucleic acid encoding the molecules providing the variant receptor or variant cytokine according to the first aspect of the invention, for example by treatment with an anti-CD3 monoclonal antibody and/or IL-2. The immune cell of the invention may be made by: (i) isolation of an immune cell-containing sample from a subject or other sources listed above; and (ii) transduction or transfection of the immune cells with one or more nucleic acid sequence(s) encoding a variant receptor or variant cytokine.

Cells can be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

The immune cells may then by purified, for example, selected on the basis of expression of the antigen-binding domain of the antigen-binding polypeptide. In certain embodiments, the cells are selected by expression of a selectable marker (e.g., a protein, a fluorescent marker, or an epitope tag) or by any method known in the art for selection, isolation and/or purification of the cells.

### Kits

This disclosure also describes kits for producing a cell expressing at least one of any of the variant receptors or variant G-CSF described herein. In certain aspects, described herein are kits comprising: cells encoding a chimeric receptor described herein, and, optionally, the cells are immune cells; and instructions for use; and, optionally, the kit comprises IL-7 and/or a variant G-CSF. In certain aspects, described herein are kits, comprising: one or more expression vector(s) comprising the nucleic acid sequence(s) encoding a chimeric receptor described herein and instructions for use; and, optionally, the kit comprises IL-7 and/or a variant G-CSF. In certain embodiments, the kits comprise at least one expression vector encoding at least one variant receptor and instructions for use. In certain aspects, the kit further comprises at least one variant cytokine in a pharmaceutical formulation or an expression vector encoding a variant G-CSF that binds to at least one of the variant receptors described herein. In certain embodiments, the kits comprise a cell comprising an expression vector encoding a variant receptor described herein.

In certain embodiments, the kits comprise a cell comprising an expression vector encoding a (CAR)/engineered T cell receptor (eTCR) or the like (e.g., engineered non-native TCR receptors). In certain embodiments the kits comprise an expression vector encoding a Chimeric Antigen Receptor (CAR)/engineered T cell receptor (eTCR) or the like. In certain embodiments the kits comprise an expression vector encoding a variant receptor described herein and a Chimeric Antigen Receptor (CAR)/engineered T cell receptor (eTCR) or the like.

In certain aspects, the kits described herein further comprise a variant cytokine. In certain embodiments, the kit further comprises at least one additional variant cytokine. In certain aspects, the kits further comprise at least one variant cytokine in a pharmaceutical formulation. In certain aspects, the kits described herein further comprise at least one a cytokine (e.g., IL-7 and/or a variant G-CSF). In some embodiments, the kits comprise at least one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent. In some embodiments, the kits comprise one or more expression vector(s) eonding at least one or more additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent.

In certain embodiments, the components are provided in a dosage form, in liquid or solid form in any convenient packaging.

In some embodiments, the kit comprises one or more expression vectors that encode at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s); In certain embodiments, the kits further comprise one or more expression vector(s) that encode a cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof. In certain embodiments, the kits further comprise one or more expression vector(s) that encodes at least one antigen binding receptor(s). In certain embodiments, the at least one antigen binding receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor, and combinations thereof. In certain embodiments, the kits further comprise an expression vector that encodes a chimeric antigen receptor.

In some embodiments, the cells further comprise one or more expression vector(s) that encode at least one cytokine(s) or chemokine(s) selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof. In some embodiments, the cells further comprise one or more expression vector(s) that encode at least one antigen binding signaling receptor(s).

In certain aspects, the at least one antigen binding signaling receptor(s) is selected from the group consisting of: native T Cell Receptor, an engineered T Cell Receptor (TCR), a Chimeric Antigen Receptors (CAR), a native B cell Receptor, an engineered B Cell Receptors (BCR), a stress ligand receptor, a pattern recognition receptor and combinations thereof. In some embodiments, the cells further comprise one or more expression vector(s) that encode at least one CAR(s), and optionally, the CAR is a mesothelin CAR. In certain embodiments, the components are provided in a dosage form, in liquid or solid form in any convenient packaging.

Additional reagents may be provided for the growth, selection and preparation of the cells provided or cells produced as described herein. For example, the kit can include components for cell culture, growth factors, differentiation agents, reagents for transfection or transduction, etc.

In certain embodiments, in additional to the above components, the kits may also include instructions for use. Instructions can be provided in any convenient form. For example, the instructions may be provided as printed information, in the packaging of the kit, in a package insert, etc. The instructions can also be provided as a computer readable medium on which the information has been recorded. In addition, the instructions may be provided on a website address which can be used to access the information.

### Methods of Selective Activation of a Variant Receptor

This disclosure provides methods for selective activation of a variant receptor expressed on the surface of a cell, comprising contacting the variant receptor described herein with a cytokine that selectively activates the chimeric receptor. In certain aspects, the cytokine that selectively activates the chimeric receptor is a variant G-CSF. The G-CSF can be a wild-type G-CSF or a G-CSF comprising one or more mutations that confer preferential binding and activation of the G-CSF to a variant receptor compared to the native (wild-type) cytokine receptor.

In certain aspects, the selective activation of the variant receptor by binding of the cytokine to the variant receptor leads to homodimerization, heterodimerization, or combinations thereof.

In certain aspects, activation of the variant receptor leads to activation of downstream signaling molecules. In certain aspects, the variant receptor activates signaling molecules or pathways that are transduced through native cellular signaling molecules to provide for a biological activity that mimics that native response, but which is specific to a cell engineered to express the variant receptor. In certain embodiments, an activated form of the chimeric receptor forms a homodimer; and, optionally, activation of the chimeric receptor causes a cellular response comprising at least one of proliferation, viability, persistence, cytotoxicity, cytokine secretion, memory, and enhanced activity of a cell expressing the receptor, and combinations thereof, and optionally, the chimeric receptor is activated upon contact with the cytokine.

In certain aspects, the activation of the downstream signaling molecules includes activation of cellular signaling pathways that stimulate cell cycle progression, proliferation, viability, and/or enhanced activity. In certain aspects, the signaling pathways or molecules that are activated are, but not limited to, JAK1, JAK2, JAK3, TYK2, STAT1, STAT2, STAT3, STAT4, STAT5A, STAT5B, STAT6, Shc, ERK1/2, IRS-1, IRS-2, and Akt. In certain aspects, activation of the variant receptor leads to increased proliferation of the cell after administration of a cytokine that binds the receptor. In certain aspects, the extent of proliferation is between 0.1-10-fold the proliferation observed when the cells are stimulated with IL-2.

In certain aspects, described herein are methods of activation of one or more chimeric receptor(s) expressed on the surface of a cell, comprising: contacting the one or more chimeric receptor(s) with IL-7 to activate the chimeric receptor; wherein the chimeric receptor comprises: (i) an extracellular domain (ECD) of IL-7Rα; (ii) a transmembrane domain (TMD); and (iii) an intracellular domain (ICD) of a cytokine receptor that is distinct from the wild-type, human IL-7Rα intracellular signaling domain set forth in SEQ ID NO: 109; wherein the ECD and TMD are each operatively linked to the ICD. In some embodiments, the chimeric receptor(s) is a chimeric receptor comprising an ICD described herein.

### Methods and Systems for Selective Activation of a Cell

In certain aspects, described herein are methods and systems for selective activation of a cell comprising (i) a receptor comprising a variant extracellular domain (ECD) of Granulocyte Colony-Stimulating Factor Receptor (G-CSFR); and (ii) a variant G-CSF that selectively binds the receptor of (i); and one or both of: (a) at least one additional agonistic or antagonistic signaling protein(s); and, optionally, the one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor. In some embodiments, the receptor is expressed on an immune cell, and, optionally the immune cell is: a T cell, and, optionally, an NK cell, and, optionally, an NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In some embodiments, the T cell is selected from the group consisting of a CD8⁺ T cell, cytotoxic CD8⁺ T cell, naïve CD4⁺ T cell, naïve CD8⁺ T cell, helper T cell, regulatory T cell, memory T cell, and γδT cell. In some embodiments, the at least one additional cytokine(s) or chemokine(s) comprises at least one of interleukin (IL)-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, and CCL19, and the receptor NKG2D, and combinations thereof. In certain embodiments, the receptor comprising a variant ECD of G-CSFR is a chimeric receptor described herein.

In certain embodiments, the system further comprises an antigen binding signaling receptor. In certain embodiments, the antigen binding signaling receptor comprises at least one of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. Any CAR known in the art could be used, including but not limited to a mesothelin CAR.

In certain aspects, described herein are methods of producing a cell expressing the receptor comprising the variant ECD of G-CSFR of a system described herein; and one or both of: (i)) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s) of a system described herein; and (ii) at least one antigen binding signaling receptor of a system described herein. In some embodiments. the method comprises introducing to the cells one or more nucleic acid(s) or expression vector(s) encoding the receptor, and one or both of (i), and (ii). In some embodiments, a first population of immune cells expresses the receptor comprising the variant ECD of G-CSFR and a second population of immune cells express the variant G-CSF In some embodiments, one or both of the first and second population(s) of immune cells further expresses one or both of: (a) at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s); and (b) at least one antigen binding signaling receptor.

In certain aspects, described herein are methods and systems for activation of a cell comprising a chimeric receptor, comprising: (a) an extracellular domain (ECD) of Interleukin Receptor alpha (IL-7Rα); (b) a transmembrane domain (TMD); and (c) an intracellular domain (ICD) of a cytokine receptor that is distinct from a wild-type, human IL-7Rα intracellular signaling domain set forth in SEQ ID NO:109; wherein the ECD and TMD are each operatively linked to the ICD.

### Methods of Adoptive Cell Transfer

The present invention provides a method for treating and/or preventing a disease which comprises the step of administering the cells expressing a variant receptor and/or a variant cytokine described herein (for example in a pharmaceutical composition as described below) to a subject.

A method for treating a disease relates to the therapeutic use of the cells described herein, e.g., T cells, NK cells, or any other immune or non-immune cells expressing a variant receptor. The cells can be administered to a subject having an existing disease or condition in order to lessen, reduce or improve at least one symptom associated with the disease and/or to slow down, reduce or block the progression of the disease. The method for preventing a disease relates to the prophylactic use of the cells of the present disclosure. Such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

In some embodiments, the subject compositions, methods and kits are used to enhance an immune response. In some embodiments the immune response is directed towards a condition where it is desirable to deplete or regulate target cells, e.g., cancer cells, infected cells, immune cells involved in autoimmune disease, etc. by systemic administration of cytokine, e.g. intramuscular, intraperitoneal, intravenous, and the like.

The method can involve the steps of: (i) isolating an immune cell-containing sample; (ii) transducing or transfecting such cells with a nucleic acid sequence or vector e.g., expressing a variant receptor; (iii) administering (i.e., infusing) the cells from (ii) to the subject, and (iv) administering a variant cytokine that stimulates the infused cells. In certain aspects, the subject has undergone an immuno-depletion treatment prior to administering the cells to the subject. In certain aspects, the subject has not undergone an immuno-depletion treatment prior to administering the cells to the subject. In certain aspects, the subject has undergone an immuno-depletion treatment reduced in severity, dose and/or duration that would otherwise be necessary without the use of the variant receptors described herein prior to administering the cells to the subject.

In certain embodiments, the method further comprises administering or providing at least one additional active agent; and, optionally, at least one additional agonistic or antagonistic signaling protein(s); and, optionally, the at least one or more additional agonistic or antagonistic signaling protein(s) comprises one or more cytokine(s), chemokine(s), hormone(s), antibody(ies) or derivative(s) thereof, or other affinity reagent(s). In certain embodiments, the subject is administered two or more populations of cells each expressing a distinct chimeric receptor and each expressing a distinct variant form of a cytokine. In certain embodiments, the cells expressing the chimeric receptor further express at least one antigen binding signaling receptor. In certain embodiments, the antigen binding signaling receptor comprises at least one receptor(s) selected from the group consisting of: a native T Cell Receptor, an engineered T Cell Receptor (TCR), a Chimeric Antigen Receptor (CAR), a native B cell Receptor, an engineered B Cell Receptor (BCR), a stress ligand receptor, a pattern recognition receptor, and combinations thereof. In certain embodiments, the antigen binding signaling receptor is a CAR. In certain embodiments, the at least one cytokine(s) or chemokine(s) is selected from the group consisting of IL-18, IL-21, interferon-α, interferon-β, interferon-γ, IL-17, IL-21, TNF-α, CXCL13, CCL3 (MIP-1α), CCL4 (MIP-1β), CD40 ligand, B cell activating factor (BAFF), Flt3 ligand, CCL21, CCL5, XCL1, or CCL19, or the receptor NKG2D, and combinations thereof.

The immune cell-containing sample can be isolated from a subject or from other sources, for example as described above. The immune cells can be isolated from a subject's own peripheral blood (1st party), or in the setting of a hematopoietic stem cell transplant from donor peripheral blood (2nd party), or peripheral blood from an unconnected donor (3rd party). The immune cells can also be derived by in vitro methods, such as induced differentiation from stem cells or other forms of precursor cell.

In some embodiments, the immune cells are contacted with the variant cytokine in vivo, i.e., where the immune cells are transferred to a recipient, and an effective dose of the variant cytokine is administered to the recipient and allowed to contact the immune cells in their native location, e.g. in lymph nodes, etc. In some embodiments, the contacting is performed in vitro. Where the cells are contacted with the variant cytokine in vitro, the cytokine is added to the cells in a dose and for a period of time sufficient to activate signaling from the receptor, which can utilize aspects of the native cellular machinery, e.g. accessory proteins, co-receptors, etc. The activated cells can be used for any purpose, including, but not limited to, experimental purposes relating to determination of antigen specificity, cytokine profiling, and for delivery in vivo.

In certain aspects, a therapeutically effective number of cells are administered to the subject. In certain aspects, the subject is administered or infused with cells expressing variant receptors on a plurality of separate occasions. In certain embodiments, at least 1×10⁶ cells/kg, at least 1×10⁷ cells/kg, at least 1×10⁸ cells/kg, at least 1×10⁹ cells/kg, at least 1×10¹⁰ cells/kg, or more are administered, sometimes being limited by the number of cells, e.g., transfected T cells, obtained during collection. The transfected cells may be infused to the subject in any physiologically acceptable medium, normally intravascularly, although they may also be introduced into any other convenient site, where the cells may find an appropriate site for growth.

In certain aspects, a therapeutically effective amount of variant cytokine is administered to the subject. In certain aspects, the subject is administered the variant cytokine on a plurality of separate occasions. In certain aspects, the amount of variant cytokine that is administered is an amount sufficient to achieve a therapeutically desired result (e.g., reduce symptoms of a disease in a subject). In certain aspects, the amount of variant cytokine that is administered is an amount sufficient to stimulate cell cycle progression, proliferation, viability and/or functional activity of a cell expressing a variant cytokine receptor described herein. In certain aspects, the variant cytokine is administered at a dose and/or duration that would is necessary to achieve a therapeutically desired result. In certain aspects, the variant cytokine is administered at a dose and/or duration sufficient to stimulate cell cycle progression, proliferation, viability and/or functional activity of a cell expressing a variant cytokine receptor described herein. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., for systemic administration, e.g., intramuscular, intraperitoneal, intravascular, and the like.

### Indications for Adoptive Cell Transfer

The present disclosure provides a cell expressing a variant receptor described herein for use in treating and/or preventing a disease. The invention also relates to the use of a cell expressing a variant receptor described herein in the manufacture of a medicament for the treatment and/or prevention of a disease.

The disease to be treated and/or prevented by the methods of the present invention can be a cancerous disease, such as, but not limited to, bile duct cancer, bladder cancer, breast cancer, cervical cancer, ovarian cancer, colon cancer, endometrial cancer, hematologic malignancies, kidney cancer (renal cell), leukemia, lymphoma, lung cancer, melanoma, non-Hodgkin lymphoma, pancreatic cancer, prostate cancer, sarcoma and thyroid cancer.

The disease to be treated and/or prevented can be an autoimmune disease. Autoimmune diseases are characterized by T and B lymphocytes that aberrantly target self-proteins, polypeptides, peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body. Autoimmune diseases include, but are not limited to, Type 1 diabetes, systemic lupus erythematosus, Rheumatoid arthritis, autoimmune thyroid diseases and Graves' disease.

The disease to be treated and/or prevented can be an inflammatory condition, such as cardiac fibrosis. In general, inflammatory conditions or disorders typically result in the immune system attacking the body's own cells or tissues and may cause abnormal inflammation, which can result in chronic pain, redness, swelling, stiffness, and damage to normal tissues. Inflammatory conditions are characterized by or caused by inflammation and include, but are not limited to, celiac disease, vasculitis, lupus, chronic obstructive pulmonary disease (COPD), irritable bowel disease, atherosclerosis, arthritis, myositis, scleroderma, gout, Sjorgren's syndrome, ankylosing spondylitis, antiphospholipid antibody syndrome, and psoriasis.

In certain embodiments, the method is used to treat an infectious disease.

In certain embodiments, the method is used to treat a degenerative disease or condition. Examples of degenerative diseases or conditions include, but are not limited to, neurodegenerative diseases and conditions related to aging.

In certain embodiments, the method is used to generate natural or engineered cells, tissues or organs for transplantation.

In certain embodiments, the condition to be treated is to prevent and treat graft rejection. In certain embodiments, the condition to be treated and/or prevented is allograft rejection. In certain aspects, the allograft rejection is acute allograft rejection.

The disease to be treated and/or prevented can involve the transplantation of cells, tissues, organs or other anatomical structures to an affected individual. The cells, tissues, organs or other anatomical structures can be from the same individual (autologous or “auto” transplantation) or from a different individual (allogeneic or “allo” transplantation). The cells, tissues, organs or other anatomical structures can also be produced using in vitro methods, including cell cloning, induced cell differentiation, or fabrication with synthetic biomaterials.

The present invention provides a method for treating and/or preventing a disease which comprises one or more steps of administering the variant cytokine and/or cells described herein (for example in a pharmaceutical composition as described above) to a subject.

A method for treating and/or preventing a disease relates to the therapeutic use of the cells of the present disclosure. Herein the cells may be administered to a subject having an existing disease or condition in order to lessen, reduce or improve at least one symptom associated with the disease and/or to slow down, reduce or block the progression of the disease. The method for preventing a disease relates to the prophylactic use of the cells of the present disclosure. Such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease. The method may involve the steps of: (i) isolating an immune cell-containing sample; (ii) transducing or transfecting such cells with a nucleic acid sequence or vector provided by the present invention; (iii) administering the cells from (ii) to a subject, and (iv) administering a variant cytokine that stimulates the infused cells. The immune cell-containing sample may be isolated from a subject or from other sources, for example as described above. The immune cells may be isolated from a subject's own peripheral blood (1st party), or in the setting of a hematopoietic stem cell transplant from donor peripheral blood (2nd party), or peripheral blood from an unconnected donor (3rd party).

Treatment can be combined with other active agents, such as, but not limited to, antibiotics, anti-cancer agents, anti-viral agents, and other immune modulating agents (e.g., antibodies against the Programmed Cell Death Protein-1 [PD-1] pathway or antibodies against CTLA-4). Additional cytokines may also be included (e.g., interferon γ, tumor necrosis factor α, interleukin 12, etc.).

### Methods Using Stem Cells Expressing Variant Cytokine Receptors

The present invention provides a method for treating and/or preventing a condition or disease which comprises the step of administering stem cells expressing a variant receptor and/or a variant cytokine described herein. In certain embodiments, stem cells expressing the variant cytokine receptors and/or variant cytokines described herein are used for regenerative medicine, cell/tissue/organ transplantation, tissue reconstruction, or tissue repair.

### Pharmaceutical Compositions

The present disclosure also relates to a pharmaceutical composition containing a plurality of cells expressing a variant receptor described herein and/or the cytokines described herein. The present disclosure also relates to a pharmaceutical composition containing a variant cytokine described herein. The cells of the invention can be formulated in pharmaceutical compositions. These compositions can comprise, in addition to one or more of the cells expressing the variant receptor described herein, a pharmaceutically acceptable excipient, carrier, buffer, stabilizer or other materials well known to those skilled in the art. Such materials should be non-toxic and should not interfere with the efficacy of the active ingredient. The pharmaceutical composition may optionally comprise one or more further pharmaceutically active polypeptides and/or compounds. Such a formulation may, for example, be in a form suitable for intravenous infusion.

For cells expressing a variant receptor, and variant cytokines described herein, according to the present disclosure that are to be given to an individual, administration is preferably in a “therapeutically effective amount” that is sufficient to show benefit to the individual. A “prophylactically effective amount” can also be administered, when sufficient to show benefit to the individual. The actual amount of cytokine or number of cells administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A pharmaceutical composition can be administered alone or in combination with other treatments, either simultaneously or sequentially dependent upon the condition to be treated.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques, cell culture, adoptive cell transfer, and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Rational Design of an Exclusive Site II Interface of G-CSF:G-CSFR(CRH)

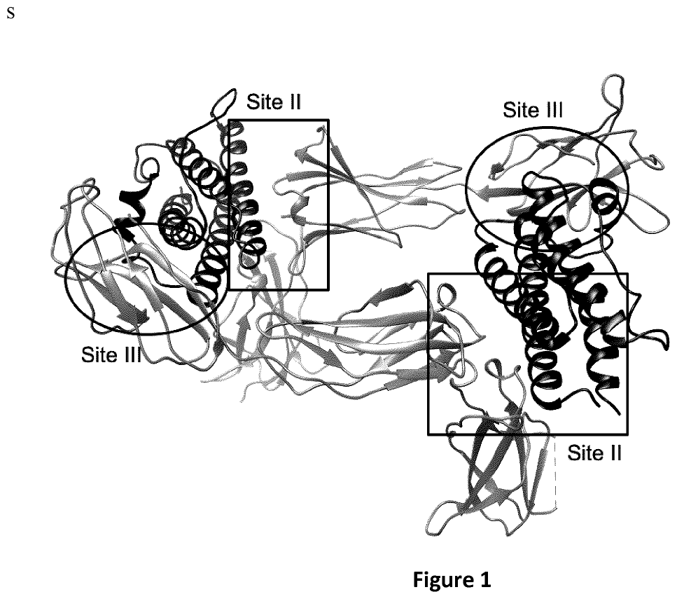

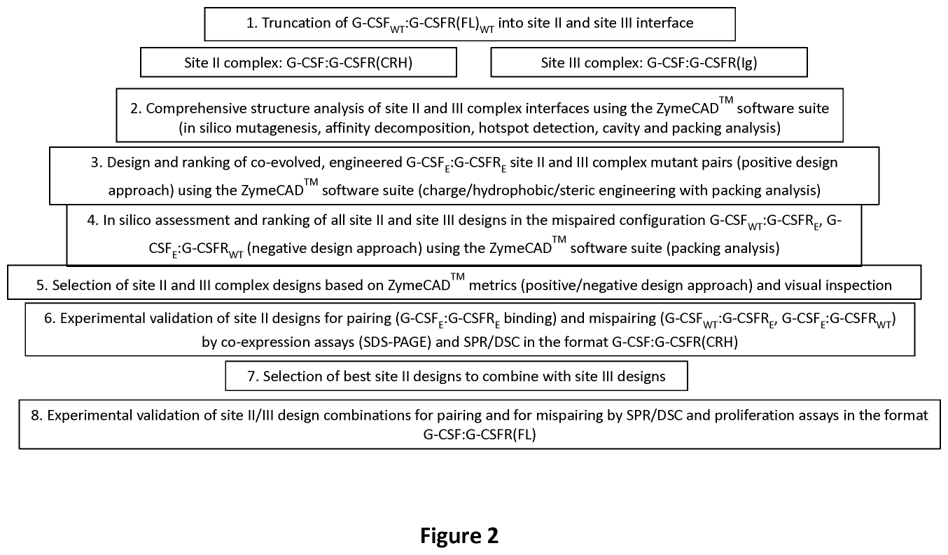

The wild type (WT) G-CSF_{WT}:G-CSFR_{WT} complex is a 2:2 heterodimer. G-CSF has two binding interfaces with the extracellular domain (ECD) of G-CSFR. The larger interface between G-CSF and the extracellular Cytokine Receptor Homologous (CRH) domain of G-CSFR is referred to as site II. The smaller interface between G-CSF and the N-terminal Ig-like (Ig) extracellular domain of G-CSFR is referred to as site III (see, FIG. 1).

To design co-evolved, engineered (E) G-CSF_{E}:G-CSFR_{E} cytokine: receptor pairs, we separated the 2:2 complex between WT G-CSF and the Ig-CRH extracellular domain of G-CSFR (Protein data Bank ID 2D9Q, Tamada et al. PNAS 2006) into the two distinct sub-complexes encompassing the site II and site III interface, consisting of G-CSF:G-CSFR(CRH) and G-CSF:G-CSFR(Ig) (see, FIG. 1), respectively, see Table 1 and Table 1A for sequences. The G-CSFR amino acid sequence set forth in SEQ ID NO. 2 of Table 1 corresponds to the amino acid positions used for the mutation numbering used herein for G-CSFR. The G-CSFR sequence set forth in SEQ ID NO. 101 listed in Table 1A is identical to SEQ ID NO. 2, but for one amino acid (glutamic acid) removed at the N-terminus. Thus, the amino acid positions for the mutation numbering used herein corresponds to amino acids 2-308 of SEQ ID NO. 101.

### Methods

To create co-evolved, exclusive G-CSF_{E}:G-CSFR_{E} mutant pairs (designs), we employed a computational design workflow (see FIG. 2). First, we created exclusive designs at site II.

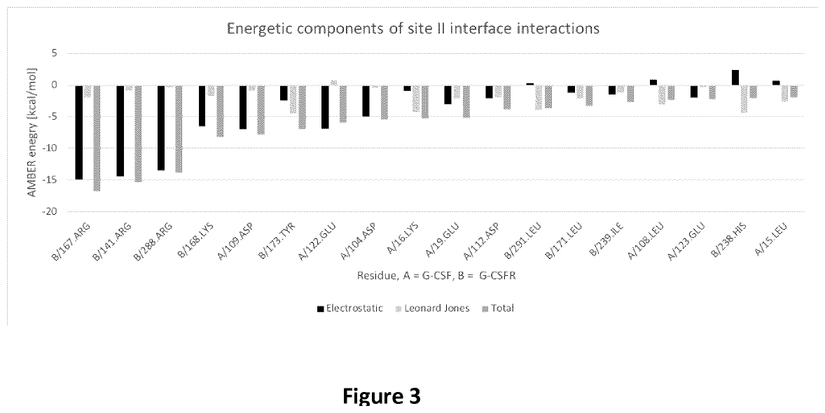

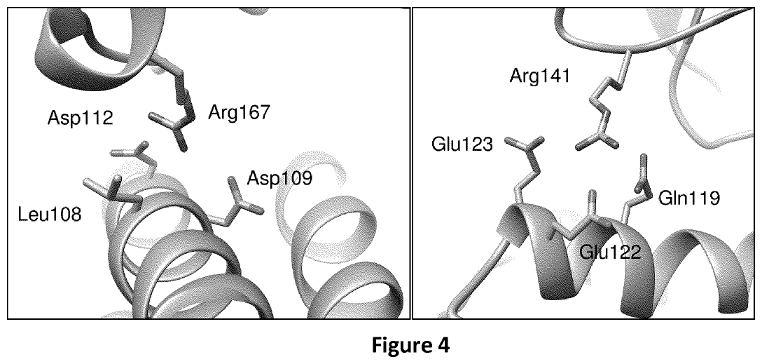

In silico structural analysis of site II interface interactions of G-CSF_{WT}:G-CSFR(CRH)_{WT} showed that most molecular interactions, that contribute to the total attractive AMBER energy at site II of 109.64 kcal/mol, are made by charged residues (see, FIG. 3). In depth inspection reveals mostly electrostatic and hydrogen-bonding interactions, for example between R167 of G-CSFR(CRH) and D112/D109 of G-CSF, which contributes 28% of the total attractive AMBER energy at site II. Another important electrostatic interaction is present between R141 of G-CSFR(CRH) and E122/E123 of G-CSF (see, FIG. 4), which contributes 21.4% of the total attractive AMBER energy at site II (See FIG. 3). Likewise, the salt bridge between E19 of the cytokine and R288 of the receptor CRH domain contributes 17.3%, the arginine is further stabilized through electrostatic and hydrogen-bonding interaction to D200 of the receptor CRH domain.

Each design at site II consists of a mutant pair of G-CSF_{E} and G-CSFR(CRH)_{E}. First, we created positive designs at site II, for example through inverting charges, by mutating basic residues to acidic residues and vice versa on the binding partner, while maintaining packing and hydrophobic interactions through additional mutations if necessary. Mutant pairs G-CSF_{E}:G-CSFR(CRH)_{E} were packed with the mean-field packing workflow of ZymeCAD™. The packed in silico models of designs at site II were visually inspected for structural integrity and they were assessed by ZymeCAD™ metrics. Specifically, we aimed to design G-CSF_{E} mutants to have ZymeCAD™ in silico dAMBER binding affinity of <10 kcal/mol for their corresponding G-CSFR(CRH)_{E} mutant (paired interaction). This metric compares AMBER affinity, the sum of Lennard Jones affinity and Electrostatics affinity, to AMBER affinity of the WT:WT cytokine-receptor pair. Designs with dAMBER_folding>80 kcal/mol were excluded. The dAMBER folding metric scores the change in the sum of Lennard Jones-bonded folding and Electrostatics folding upon mutation. We also excluded designs that scored higher than 400 kcal/mol in dDDRW apostability. This metric describes the change in knowledge-based potential stability upon mutation of a protein from its apo form.

Next we packed G-CSF_{E} mutants of each design in a complex with WT G-CSFR (and vice versa G-CSFR_{E} with G-CSF_{WT}) with ZymeCAD™, to assess the metrics of each positive design under conditions of mispairing when the following two complexes would form: G-CSF_{WT}:G-CSFR(CRH)_{E} and G-CSF_{E}:G-CSFR(CRH)_{WT}. We calculated in silico ddAMBER metrics for the mispaired orientations with ZymeCAD™ (DdAMBER_affinity_Awt_Bmut is the AMBER affinity of the paired, engineered complex subtracted by the AMBER affinity of the mispaired complex G-CSF_{WT}:G-CSFR(CRH)_{E}, ddAMBER_affinity_Amut_Bwt is the AMBER affinity of the paired, engineered complex subtracted by the AMBER affinity of the mispaired complex G-CSF_{E}:G-CSFR(CRH)_{WT}. Designs that minimized mispaired ddAMBER affinity metrics were considered to be more selective for their paired binding partner over binding to wild type cytokine or receptor binding.

All site II designs were clustered and the packing metrics of G-CSF_{E}:G-CSFR(CRH)_{E}, G-CSF_{WT}:G-CSFR(CRH)_{E}, G-CSF_{E}:G-CSFR(CRH)_{WT} were considered to assess the strength of pairing (positive design) and selectivity against mispairing with WT G-CSF and G-CSFR(CRH) (negative design) and rank designs.

### Results

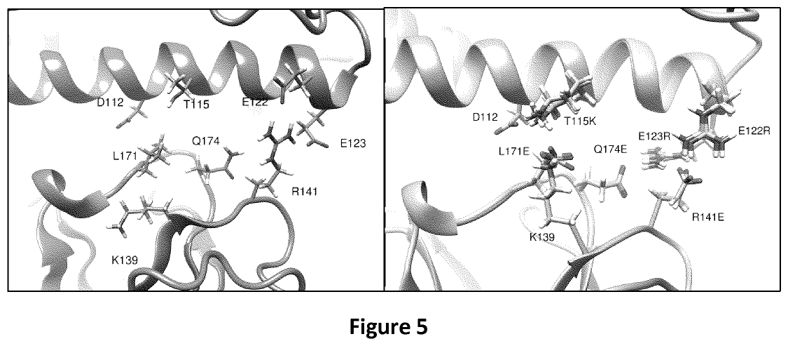

Designs listed in Table 2 have packing metrics in ZymeCAD™ that favour pairing of G-CSF_{E}:G-CSFR(CRH)_{E} in silico (see, Table 5) and showed satisfying interactions in silico in the visual inspection such as the presence of salt bridges, hydrogen bonds and absence of severe clashes (see, FIG. 5). These designs also showed high selectivity against mispairing with WT G-CSF or G-CSFR(CRH) (see, Table 5).

Therefore, the site II mutations of the same variant G-CSF and receptor designs shown in Table 2 are predicted to exhibit preferential binding, over wild type G-CSFR and G-CSF, respectively.

TABLE 5
Site II designs with AMBER metrics in kcal/mol from triplicate
in silico mean-field packs with ZymeCAD ™.

| | G-CSF_{E}:G- | | G-CSF_{E}:G- | G-CSF_{WT}:G- |
|---|---|---|---|---|
| | CSFR(CRH)_{E} | | CSFR(CRH)_{WT} | CSFR(CRH)_{E} |
| Design | dAMBER_aff | dAMBER_folding | ddAMBER_aff_E_WT | ddAMBER_aff_WT_E |
| 1 | −13.2 | 36.0 | −35.2 | −47.3 |
| 2 | −24.2 | 35.6 | −41.0 | −55.8 |
| 6 | 8.9 | −7.5 | −8.8 | −13.3 |
| 7 | −24.1 | −7.8 | −45.8 | −51.7 |
| 8 | −30.5 | 12.2 | −73.5 | −86.5 |
| 9 | −25.1 | 17.4 | −89.6 | −119.5 |
| 15 | −53.3 | 59.5 | −72.4 | −71.0 |
| 17 | −0.2 | 19.3 | −23.2 | −28.5 |
| 18 | −17.6 | 75.2 | −44.2 | −27.8 |
| 19 | 20.6 | 38.1 | −10.7 | −17.3 |
| 20 | −9.9 | 28.5 | −52.7 | −64.4 |
| 21 | −18.0 | 51.5 | −55.4 | −47.8 |
| 22 | −32.3 | 52.2 | −75.0 | −53.3 |
| 24 | −13.0 | 57.7 | −50.8 | −86.4 |
| 25 | −49.8 | 83.5 | −100.2 | −91.3 |
| 27 | −60.5 | 43.0 | −131.4 | −156.4 |
| 28 | −61.6 | 82.6 | −148.8 | −135.9 |
| 30 | 9.6 | −5.0 | −31.4 | −29.1 |
| 34 | 2.9 | 16.7 | −60.4 | −63.1 |
| 35 | −27.4 | 0.3 | −50.1 | −63.0 |
| 36 | −92.2 | 35.1 | −110.7 | −93.5 |
| 42 | −82.1 | 67.7 | −137.0 | −119.7 |
| 43 | −14.9 | 51.0 | −87.4 | −111.2 |
| 44 | −20.2 | 53.6 | −86.4 | −108.4 |
| 45 | −35.5 | 38.1 | −70.7 | −107.0 |
| 46 | −11.6 | 44.2 | −75.0 | −95.8 |
| 47 | −27.8 | 75.8 | −72.4 | −91.5 |
| 48 | −69.7 | 76.8 | −127.8 | −87.7 |
| 49 | −55.9 | 79.3 | −94.7 | −84.7 |
| 50 | −52.0 | 38.6 | −123.5 | −134.9 |
| 80 | 15.3 | 13.0 | −16.2 | 10.8 |
| 81 | −2.4 | 52.7 | −24.5 | −28.9 |
| 82 | 29.8 | 10.9 | 1.6 | 0.7 |
| 83 | 36.3 | 54.6 | −21.6 | 5.5 |

### Example 2: In Vitro Screening of Site II Designs

Selected site II designs were screened in a pulldown experiment in the format G-CSF_{E}:G-CSFR(CRH)_{E} for their ability to form a site II complex when co-expressed in a Baculovirus based insect cell system. We also assessed whether designs could form a mispaired complex with WT receptor or cytokine by co-expressing each designs G-CSF_{E} mutant with G-CSFR(CRH)_{WT}, and vice versa each design G-CSFR(CRH)_{E} mutant was co-expressed with GCSF_{WT}. The expression of G-CSF_{E} alone was verified by single infections of the cytokine mutant.

### Methods

In brief, site II G-CSF designs and corresponding, paired G-CSFR(CRH) mutants were cloned separately into insect cell transfection vectors. G-CSF WT (residues 1-173, Table 1) and mutants were cloned into a modified pAcGP67b transfer vector (Pharmingen), in frame with an N-terminal secretion signal and a C-terminal TEV-cleavable Twin Strep tag with the sequence AAAENLYFQ/GSAWSHPQFEKGGGSGGGSGGSAWSHPQFEK (SEQ ID NO.230). Of the receptor extracellular domain, only the CRH domain (residues 98-308, Table 1) with site II design mutations was cloned into a modified pAcGP67b transfer vector, in frame, with an N-terminal secretion signal and TEV-cleavable Hexahistidine tag of the sequence HHHHHHSSGRENLYFQ/GSMG (SEQ ID NO.231). All constructs were synthesized and codon optimized for insect cell expression (Genscript). Transfer vector DNA was prepared by Midi-prep (ThermoScientific, cat. K0481), was endotoxin-free and had a A_{260/280} absorbance ratio of 1.8-2.0. Recombinant virus generation was achieved by co-transfection of recombinant, linearized Baculovirus DNA with the vector DNA in Spodoptera frugiperda 9 (Sf9) cells, using the adherent method, as described by the manufacturer (Expression Systems, California). Approximately 1 h prior to transfection, 2 mL of healthy, log-phase Sf9 cells at 0.46×10⁶ cells ml⁻¹ were seeded per well of a 6-well tissue culture plate (Greiner, cat. 657-160). Transfection mixtures were prepared as follows: 100 μl Transfection Medium (Expression Systems, California, cat. 95-020-100) was placed into each of two sterile 1.5 ml microfuge tubes, A and B. To Tube A, 0.4 μg recombinant BestBac 2.0 Δv-cath/chiA linearized DNA (Expression Systems, California, cat. 91-002) and 2 μg vector DNA was added. To Tube B, 1.2 μl 5× Express²TR transfection reagent (Expres2ION, cat. 52-55A-001) was added. Solutions A and B were incubated at approximately 24° C. for 5 minutes and then combined and incubated for 30 minutes. After incubation 800 μl Transfection Medium was added to each transfection reaction to increase the volume to 1 ml. The old ESF 921 medium was removed from the wells and replaced with 1 mL transfection mixture applied drop-by-drop so as not to disturb the cell monolayer. Plate(s) were gently rocked back-and-forth and from side-to-side to evenly distribute the transfection mixtures, and incubated at 27° C. for 4 h. After 4 h, the transfection mixture was removed from the co-transfection plate(s) and 2 mL fresh ESF 921 insect cell culture medium (Expression Systems, California, cat. 96-001-01) containing gentamicin at 10 μg/ml (cat. 15750-060) was added drop-by-drop. To prevent evaporation, plates were wrapped in saran wrap, placed in a sterile plastic box and incubated for 4-5 days at 27° C. At day 4 or 5 post-transfection P1 supernatants were collected and clarified by centrifugation at 5000 rpm for 5 minutes and transferred to a new sterile tube and stored at 4° C., protected from light.

The recombinant P1 stocks produced as described above were further amplified to high-titre, low passage P2 stocks for protein expression studies. The following workflow produced 50-100 mL virus using the P1 seed stock of virus harvested from the co-transfection as inoculum. 50 mL of log-phase Sf9 cells at 1.5×10⁶ cells ml⁻¹ were seeded into a 250 mL shake flask (FisherScientific, cat. PBV 250), and 0.5 mL P1 virus stock was added. Cells were incubated at 27° C., with shaking at 135 rpm and monitored for infection. P2 virus supernatant was harvested 5-7 days post infection and clarified by centrifugation at 4000 rpm for 10 minutes. To minimize titre loss, 10% heat-inactivated FBS (VWR, cat. 97068-085) was added and P2 virus stored at 4° C. in the dark.

The P2 viral stocks were tested for protein expression in small-scale. P2 virus was used to co-infect G-CSF_{E} mutants with their corresponding G-CSFR_{E} mutant in Trichoplusia ni (Tni) cells in 12-well plates. Separate co-infections for each design mutant were performed using the P2 stocks of G-CSF_{WT} and GCSFR(CRH)_{WT}, as well as for G-CSF_{E} mutants alone. For each reaction, 2 mL of healthy log-phase Tni at 2×10⁶ cells ml⁻¹ was inoculated with 20 μl P2 virus. Plates were incubated at 27° C. for approximately 70 h with shaking at 135 rpm. Supernatants were clarified by centrifugation at 5000 rpm for 3 minutes and secreted protein was pulled down, via the Twin-Strep Tag (TST) on G-CSF_{E} mutants and G-CSF_{WT}, in batch mode with streptactin-XT superflow (IBA Lifesciences, cat. 2-4010-010). Briefly, to each 1.8 mL reaction supernatant 0.2 mL 10× HEPES Buffered Saline (HBS: 20 mM HEPES pH8, 150 mM NaCl) was added to 1×, 20 μl bed volume (b.v) of purification beads added and reactions incubated for 30 minutes with end-over-end mixing at 24° C. An additional 20 μl b.v of purification beads was added followed by a second 30 minute incubation. Beads were pelleted by centrifugation at 2200 rpm for 3 minutes, supernatant was removed and the beads washed with 1× HBS buffer. Protein was eluted with 30 μl BXT elution buffer (100 mM Tris-CL pH8, 150 mM NaCl, 1 mM EDTA, 50 mM Biotin, (IBA Lifesciences, cat. 2-1042-025)), boiled with SDS-PAGE sample buffer and analyzed on a 12% Bolt Bis-Tris plus, 12 well gel (Thermo Fisher Scientific, cat. NW00122BOX) run at 200V for 30 minutes under reducing conditions.

### Results

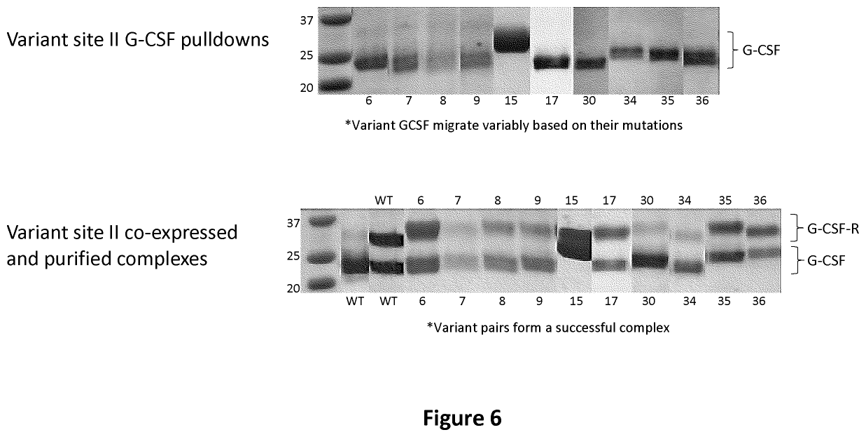

Site II designs #6, 7, 8, 9, 15, 17, 30, 34, 35, 36 showed expression for G-CSF_{E} alone, and formed sufficiently stable, paired G-CSF_{E}:G-CSFR_{E} complexes that were pulled down through the Twin Strep tag on G-CSF_{E} (see, FIG. 6). For example, site II design #6 post pulldown of the engineered complex showed a band on SDS-PAGE for its G-CSF_{E} mutant at ˜22 kDa as well as for its corresponding, co-expressed G-CSFR(CRH)_{E} mutant at ˜33 kDa (see, FIG. 6).

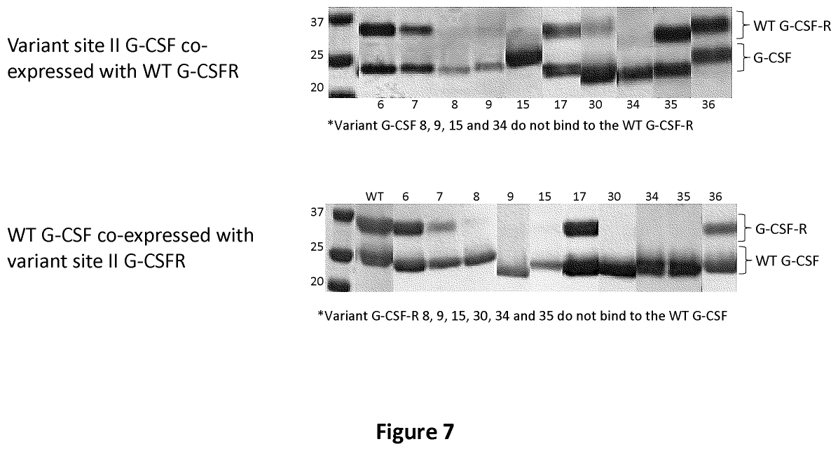

Site II designs #8, 9, 15 and 34 were also selective against mispairing with WT G-CSF and WT G-CSFR (see, FIG. 7) in the co-expression assay because they were not pulled down by WT G-CSF (see, FIG. 7 bottom panel), and WT receptor was not pulled down by the G-CSF_{E} mutant (see, FIG. 7 top panel). Site II G-CSFR_{E} design 30 and 35 were selective against mispairing with the WT G-CSF in the co-expression assay because they were not pulled down by WT G-CSF (see, FIG. 7 bottom panel), however, the reciprocal G-CSF_{E} design was not selective against mispairing with the WT G-CSFR in co-expression assay because WT G-CSFR was pulled down (see, FIG. 7 top panel). Site II designs 6, 7, 17 and 36 were not selective against mispairing with the WT G-CSF or WT G-CSFR (see, FIG. 7) in the co-expression assay because they pulled down WT G-CSFR and were pulled down by WT G-CSF.

Site II receptor mutants with mutations at residue R288 did not express in the G-CSFR(CRH) receptor chain format without Ig domain. R288 containing designs were evaluated for paired complex formation by SPR in combination with site III designs on the Ig domain (see Example 6). Therefore, several site II designs were identified that formed sufficiently stable, paired G-CSF_{E}:G-CSFR_{E} complexes that were also selective against mispairing with WT G-CSF and WT G-CSFR.

Therefore, various site II mutations of the same variant G-CSF and receptor designs shown in Table 2 exhibit preferential binding over wild type G-CSFR and G-CSF, respectively.

### Example 3: Rational Design of an Exclusive Site III Interface of G-CSF:G-CSFR(Ig)

The avidity effect of the site III receptor Ig domain binding to G-CSF contributes to the formation of the 2:2 heterodimer stoichiometry of the G-CSF:G-CSFR(Ig-CRH) complex (see, FIG. 1). A selective design present only at site II with a WT site III interface appears to be insufficient to create a fully exclusive 2:2 G-CSF_{E}:G-CSFR(Ig-CRH)_{E} complex. To favor binding of a paired, co-evolved design to be selective over mispaired binding to WT cytokine or receptor, we applied the in silico design workflow described in Example 1 to the site III interface to create selective site III designs (see FIG. 2).

### Methods

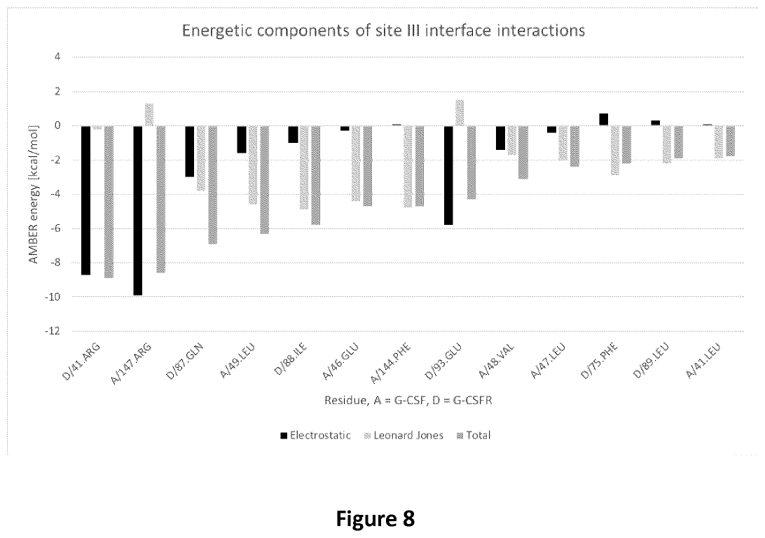

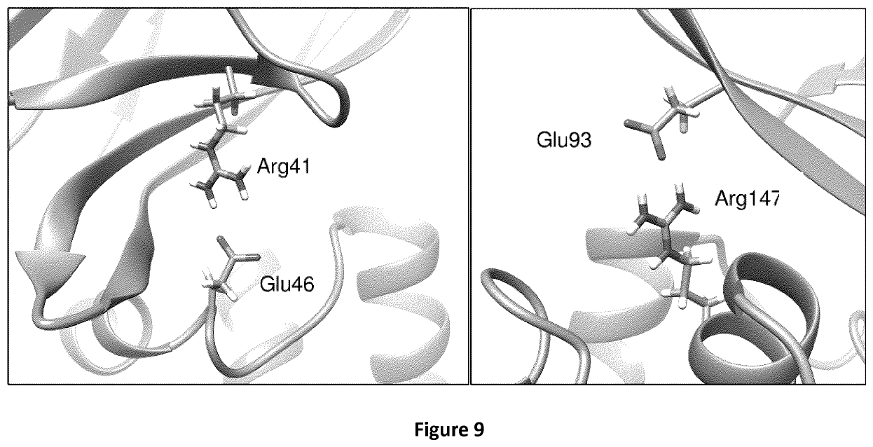

First, we conducted a structural analysis of site III interface interactions. The site III interface contributes 55.64 kcal/mol AMBER energy to the G-CSF:G-CSFR complex and it has an overall smaller interface area with 571.5 Å² compared to site II with an interface area of 692.6 Å². In depth inspection of site III interface reveals less electrostatic and hydrogen-bonding interactions compared to the site II interface (see, FIG. 8). Key interactions at site III are for example a salt bridge between E46 of G-CSF and R41 of the receptor Ig domain, furthermore between R147 of G-CSF and E93 of the receptor Ig domain (see, FIG. 9). Both interactions contribute 15.9% and 15.4%, respectively, to the total attractive AMBER energy of site III. Further interactions are for example made through Q87 of the receptor Ig domain, which forms a hydrogen-bonding interaction with its side chain amide to the backbone of G-CSF site III, and has Lennard Jones interactions with surrounding side chains of the cytokine such as E46 and L49. This interaction makes up 12.3% of the total attractive AMBER energy at site III.

Next, we created positive designs at site III where G-CSF_{E} mutant has a favorable AMBER binding affinity in silico for its co-evolved G-CSFR(Ig)_{E} mutant (paired interaction). This was done for example through inverting charges or changing shape complementarity while maintaining favorable Lennard Jones and hydrogen bonding interactions. Mutant pairs G-CSF_{E}:G-CSFR(Ig)_{E} were packed with the mean-field packing workflow of ZymeCAD™. The packed in silico models of designs at site III were visually inspected for structural integrity and they were assessed by ZymeCAD™ metrics as described in Example 1. Next, we packed G-CSF_{E} mutants of each design with WT G-CSFR(Ig) (and vice versa G-CSFR(Ig)_{E} with G-CSF_{WT}) with ZymeCAD™ to assess the metrics under conditions of mispairing with WT cytokine and receptor. We calculated ddAMBER affinity metrics (G-CSF_{WT}:G-CSFR(Ig)_{E}, G-CSF_{E}:G-CSFR(Ig)_{WT}) for site III designs with ZymeCAD™ (see Table 6) as described before for site II in Example 1.

Site III designs were clustered and the packing metrics of all three in silico complexes G-CSF_{E}:G-CSFR(Ig)_{E}, G-CSF_{WT}:G-CSFR(Ig)_{E}, G-CSF_{E}:G-CSFR(Ig)_{WT} were considered together with visual inspection to assess the strength of pairing and selectivity against mispairing with WT in order to rank designs.

### Results

Designs listed in Table 3 have in silico packing metrics in ZymeCAD™, that favour pairing of G-CSF_{E}:G-CSFR(Ig)_{E}, with high selectivity over mispairing with WT G-CSF or G-CSFR(Ig).

Therefore, various site III mutations of the same variant G-CSF and receptor designs shown in Table 3 are predicted to exhibit preferential binding, over wild type G-CSFR and G-CSF, respectively.

TABLE 6
Site III designs with AMBER metrics from triplicate in silico
mean-field packs in kcal/mol in ZymeCAD ™.

| | G-CSF_{E}:G- | | G-CSF_{E}:G- | G-CSF_{WT}:G- |
|---|---|---|---|---|
| | CSFR(Ig)_{E} | | CSFR(Ig)_{WT} | CSFR(Ig)_{E} |
| Design # | dAmber_affinity | dAmber_folding | ddAmber_affinity_E_WT | ddAmber_affinity_WT_E |
| 51 | −31.84 | 21.42 | −28.60 | −49.04 |
| 52 | −31.81 | 21.57 | −36.14 | −42.08 |
| 53 | −28.72 | 30.24 | −36.29 | −48.49 |
| 54 | −27.30 | 36.15 | −34.29 | −31.51 |
| 55 | −23.32 | 21.36 | −33.71 | −37.62 |
| 56 | −21.25 | 28.70 | −40.40 | −36.60 |
| 57 | −18.31 | 15.77 | −24.68 | −51.81 |
| 58 | −17.69 | 17.14 | −24.62 | −36.77 |
| 59 | −17.64 | 28.39 | −27.76 | −40.42 |
| 60 | −16.01 | 30.54 | −22.94 | −45.27 |
| 61 | −15.84 | 29.64 | −9.07 | −33.76 |
| 62 | −14.29 | 2.89 | −21.18 | −12.78 |
| 63 | −14.20 | 3.57 | −14.42 | −14.65 |
| 64 | −13.68 | 34.79 | −22.85 | −22.93 |
| 65 | −13.51 | 28.01 | −15.43 | −34.93 |
| 66 | −12.58 | 10.88 | −19.51 | −26.53 |
| 67 | −11.43 | 35.72 | −21.79 | −27.46 |
| 68 | −10.95 | 15.99 | −12.52 | −20.20 |
| 69 | −9.53 | 18.46 | −7.37 | −32.23 |
| 70 | −9.34 | 13.13 | −27.74 | −10.28 |
| 71 | −7.63 | 15.05 | −7.95 | −15.03 |
| 72 | −5.68 | 10.95 | −1.97 | −16.54 |

### Example 4: Combination of Site II and III to Create a Variant, Co-Evolved Cytokine-Receptor Switch

To develop a fully selective design pair G-CSF_{E}:G-CSF(Ig-CRH)_{E}, that enables binding and signaling through the 2:2 heterodimeric engineered complex and has low or completely abolished cross-reactivity with wild type cytokine or receptor, selected site II and III designs from Examples 1 and 3 were combined (see, Table 4) and tested in vitro.

In the same way the designs in Table 4 were combined, any other combination of a site II design of Example 1 (Table 2) with a site III design of Example 3 (Table 3) could result in a combined fully selective G-CSF_{E}:G-CSFR(Ig-CRH)_{E} design that enables variant signaling. Combination designs 401 and 402 were tested in the co-expression assay described in Example 2 for their ability to form an engineered G:CSF_{E}:G-CSFR(Ig-CRH)_{E} complex, as well as for their ability to bind WT cytokine or receptor.

### Methods

The co-expression assay with pulldown through the cytokine TST tag was performed as described above in Example 2, with the difference that the receptor constructs contained the Ig and CRH domains (residues 2-308, Table 1) referred to as G-CSFR(Ig-CRH).

### Results

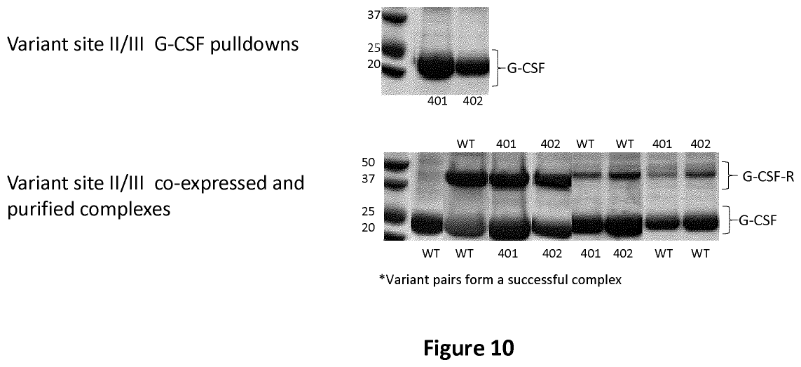

Combination designs 401 and 402 were fully selective in the co-expression assay, in that the design cytokines pulled down their co-evolved, engineered receptor but not WT receptor, and vice versa WT G-CSF did not pull down the engineered receptor (see, FIG. 10).

These results show that variant G-CSF and receptors comprising variant G-CSFR ECD designs combining select site II and site III mutations are capable of specific binding to engineered cytokine receptor pair and do not bind the wild type receptor or cytokine, respectively.

### Example 5: Production of G-CSF and G-CSFR Wildtype and Mutants

To produce wild type and engineered cytokine and receptor variants and compare their biophysical properties, recombinant proteins were expressed and purified from insect cells.

### Methods

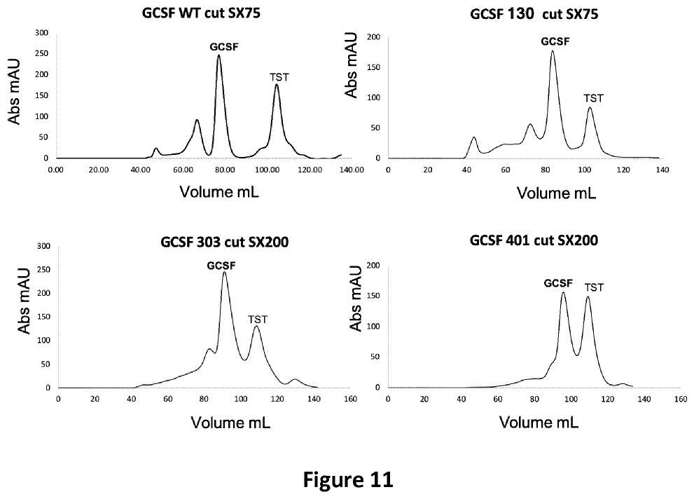

G-CSF_{E} and G-CSF_{WT} were cloned as described above. Preparative scale production of recombinant proteins was performed in 2-4 L healthy, log-phase Tni cells as follows: 800 mL Tni at 2×10⁶ cells ml⁻¹ were inoculated with 20 μl cytokine variant P2 virus per 2 ml cells, and incubated for 70 h at 27° C. with shaking at 135 rpm. After incubation, the cells were pelleted by centrifugation at 5500 rpm for 15 minutes and the supernatant filtered twice, first through a 1 μm Type A/E glass fiber filter (PALL, cat. 61631) followed by 0.45 μm PVDF membrane filter (Sigma Aldrich, cat. HVLP04700). Protease inhibitor cocktail III (Sigma Aldrich, cat. 539134) was added and the supernatant buffer exchanged into HBS (20 mM HEPES pH8, 150 mM NaCl) and concentrated to 300 mL on the tangential flow. Protein was purified in batch mode with 3×3 mL b.v Streptactin-XT superflow and incubated for 2×1 h, and 1× overnight at 4° C. with stirring. Prior to elution the resin was washed with 10 CV HBS buffer. Protein was eluted in 4×5 mL of BXT elution buffer. Eluates were analyzed by nanodrop A280 and reducing SDS-PAGE, concentrated to −2 mL and the TST purification tag cleaved by incubation with TEV at 1:80 ratio of TEV:protein at 18° C. overnight with end-over-end mixing. Cut protein was confirmed by SDS-PAGE prior to being loaded onto either a SX75 16/600 or SX200 16/600 size exclusion column (GE Healthcare, cat. 28-9893-33 or 28-9893-35) equilibrated in 20 mM BisTris pH 6.5, 150 mM NaCl (see, FIG. 11). Protein containing fractions were analyzed by reducing SDS-PAGE, pooled and the concentration was measured by nanodrop A280 measurement.

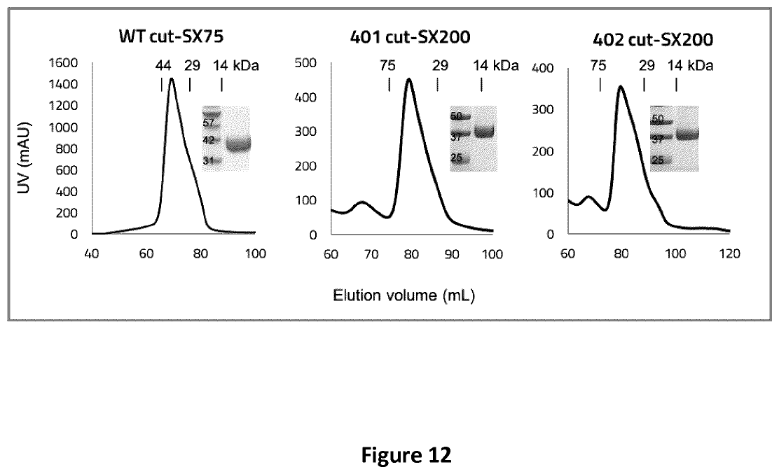

For protein purification of receptor variants, wild type and G-CSFR(Ig-CRH)_{E} mutants were cloned as described above, receptor constructs for purification included the Ig domain in addition to the CRH domain (residues 3-308 of Uniprot ID Q99062, Table 1). Viral stocks were prepared and used for infection at 2-4 L scale as described above. Clarified supernatants were buffer exchanged into Ni-NTA binding buffer (20 mM HEPES pH8, 1 M NaCl, 30 mM Imidazole) and concentrated to 300 mM as described above. Protein was purified in batch bind mode with 3×3 mL b.v Ni-NTA superflow and incubated for 2×1 h, and 1× overnight at 4° C. with stirring. Prior to elution the resin was washed with 10 CV binding buffer. Protein was eluted in 4×5 mL Ni-NTA elution buffer (20 mM HEPES pH8, 1 M NaCl, 250 mM Imidazole). Eluates were analyzed, buffer exchanged into 20 mM Bis-Tris pH 6.5, 150 mM NaCl, concentrated and cleaved overnight as described above. Cut protein was subsequently loaded onto a SX 75 16/600 or SX200 16/600 size exclusion column (GE Healthcare) equilibrated in 20 mM BisTris pH 6.5, 150 mM NaCl (see, FIG. 12). Protein containing fractions were analyzed by reducing SDS-PAGE, pooled and the concentration was measured by nanodrop A280 measurement.

### Results

Wild type, G-CSF_{E} and G-CSFR_{E} mutants were >90% pure post SEC as judged by reducing SDS-PAGE (see, FIGS. 11 and 12). The yield post SEC per 1 L culture of design 401 and 402 G-CSF_{E} was 2.7 mgs and 1.6 mgs, respectively. The yield post SEC per 1 L production of design 401 and 402 G-CSFR_{E} was 1.7 mgs and 1.5 mgs, respectively. WT G-CSF was purified with a yield of 2.1 mgs post SEC per 1 L culture, WT G-CSFR was purified with a yield of 3.1 mg post SEC per 1 L of culture.

These results confirm that the methods used to purify variant G-CSF and receptors were efficient to yield purified protein to be used for analysis of biophysical properties in vitro.

### Example 6: Determination of the Affinity of Designs for their Paired and Mispaired Binding Partner by SPR

In order to determine the affinity of design cytokines for their co-evolved receptor mutant, we measured the affinity of G-CSF_{E} for G-CSFR_{E}. We also determined the affinity of G-CSF_{E} for G-CSFR_{WT} and the affinity of G-CSF_{WT} for G-CSFR_{E} for a subset of designs by SPR (mispaired).

### Methods

The SPR binding assays were carried out on a Biacore T200 instrument (GE Healthcare, Mississauga, ON, Canada) with PBS-T (PBS+0.05% (v/v) Tween 20) running buffer at a temperature of 25° C. CM5 Series S sensor chip, Biacore amine coupling kit (NHS, EDC and 1 M ethanolamine), and 10 mM sodium acetate buffers were all purchased from GE Healthcare. PBS running buffer with 0.05% Tween20 (PBS-T) was purchased from Teknova Inc. (Hollister, CA). Designs were assessed in three different immobilization orientations.

To determine binding affinity of G-CSF_{E} to G-CSFR_{E} the G-CSFR_{E} mutant was captured by standard amine coupling as described by the manufacturer (GE LifeSciences). Briefly, immediately after EDC/NHS activation, a 5 μg/mL solution of G-CSFR_{E} in 10 mM NaOAc, pH 5.0, was injected at a flow rate of 5 μL/min until a receptor density of ˜700-900 RU was reached. The remaining active groups were quenched by a 420 s injection of 1 M ethanolamine hydrochloride-NaOH pH 8.5 at 10 μL/min. Using single-cycle kinetics, six concentrations of a two-fold dilution series of the corresponding G-CSF_{E} mutant starting at 200 nM with a blank buffer control were sequentially injected at 25 μL/min for 300 s with a 1800 s dissociation phase, resulting in a set of sensorgrams with a buffer blank reference. The same sample titration was also performed on a reference cell with no variants captured. The chip was regenerated to prepare for the next injection cycle by one pulse of 10 mM glycine/HCl, pH 2.0, for 30 s at 30 μL/min.

To assess binding affinity of G-CSF_{WT} to G-CSFR_{E}, G-CSF_{E} was captured on the chip at a density of ˜700-900 RU as described above. Using single-cycle kinetics, six concentrations of a two-fold dilution series of each G-CSFR_{WT} starting at 200 nM with a blank buffer control were sequentially injected at 25 μL/min for 300 s with a 1800 s total dissociation time, resulting in a set of sensorgrams with a buffer blank reference. The same sample titration was also performed on a reference cell with no variants captured and the chip was regenerated as described above.

To assess binding affinity of G-CSF_{E} to G-CSFR_{WT}, recombinant G-CSFR_{WT} purified as described in Example 5 was captured as described above in this Example. Using single-cycle kinetics, six concentrations of a two-fold dilution series of each G-CSF_{E} mutant starting at 200 mM with a blank buffer control were sequentially injected as described above. The same sample titration was also performed on a reference cell with no variants captured. The G-CSFR_{WT} surface was regenerated as described above.

As a control, the binding of WT G-CSF to WT G-CSFR(Ig-CRH) was assessed in each experiment and used to calculate the K_{D} fold change within each independent measurement.

Double-referenced sensorgrams from duplicate or triplicate repeat injections were analyzed using Biacore™ T200 Evaluation Software v3.0 and fit to the 1:1 Langmuir binding model.

### Results

Kinetic-derived affinity constants (K_{D}) where obtained by fitting the association and dissociation phases of the curves. The K_{D} of WT G-CSF for WT G-CSFR(Ig-CRH) ranged between 1.8-2.5E-9. For the cases in which the kinetic parameters could not be fit an attempt was made to derive a steady state affinity constant. In those cases, the KD fold change was calculated from the steady state derived K_{D} for the WT G-CSF:WT G-CSFR(Ig-CRH) pair and is indicated in Table 7.

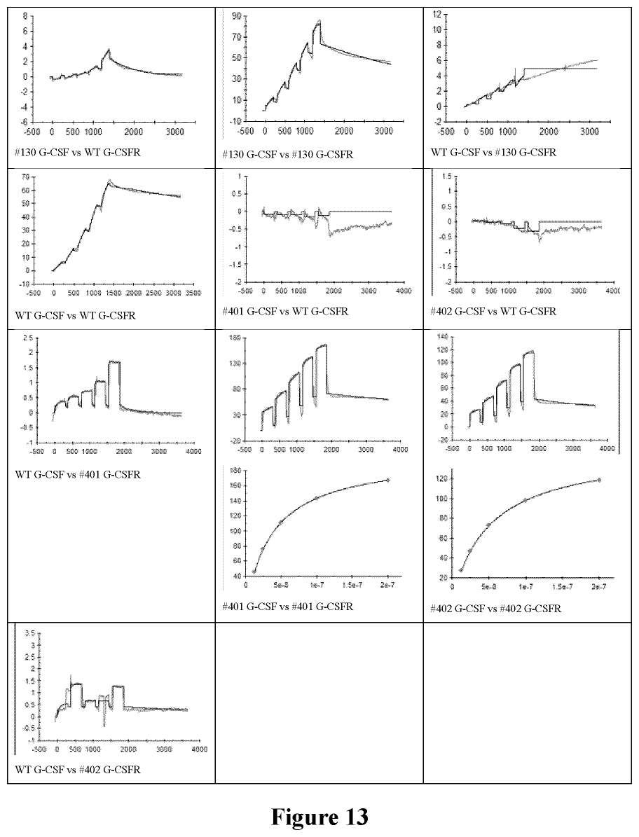

Designs 9, 130, 134, 137, 307, 401 and 402 showed affinities for their co-evolved binding partner not more than 2× different from the WT:WT KD (see Table 7 and FIG. 13).

Design 9, 30 and 34 G-CSFR(Ig-CRH)_{E} mutants showed more than >700× weaker affinity for WT G-CSF compared to WT G-CSFR(Ig-CRH). Design #35 G-CSFR(Ig-CRH)_{E} was less selective against mispairing with WT cytokine, its affinity for WT G-CSF was reduced ˜19× compared to WT:WT affinity. Designs 130, 134, 401, 402, 300, 3003, 304 and 307 G-CSFR(Ig-CRH)_{E} mutant were the most selective against mispairing with WT G-CSF and did not show appreciable binding to WT G-CSF at the concentrations titrated (see, Table 8, FIG. 13).

Design 124, 130, 401, 402, 300, 303, 304 and 307 G-CSF_{E} mutants did not show appreciable binding to WT G-CSFR(Ig-CRH) at the concentrations titrated. Design 9, 30 and 34 G-CSF_{E} mutants showed at least a ˜20× weaker K_{D} for WT G-CSFR(Ig-CRH) compared to the WT:WT KD. Design #134 G-CSF_{E} showed ˜500× weaker affinity for WT G-CSFR(Ig-CRH) (see, Table 9, FIG. 13). Design 35 and 117 G-CSF_{E} mutants show binding to the WT G-CSFR(Ig-CRH) similar to the WT:WT KD.

These results show that selected variant G-CSF designs do not bind wild type G-CSFR ECD or bind with a significantly reduced affinity to wild type G-CSFR ECD (at least ˜20× weaker KD).

TABLE 7
Change in binding affinity (KD) of G-CSF_{E} mutants
for their corresponding G-CSFR(Ig-CRH)_{E} mutant compared
to WT:WT binding affinity determined by SPR

| | K_{D} fold change |
|---|---|
| WT:WT control | — |

Design G-CSF_{E}:G-CSFR(Ig-CRH)_{E}

| | |
|---|---|
| 9 | 1.2 |
| 30 | 11 |
| 34 | 5.0 |
| 35 | 3.3 |
| 130 | 0.9 |
| 134 | 1.0 |
| 137 | 0.3 |
| 401 | 0.6^{ss} |
| 402 | 0.8^{ss} |
| 300 | 194 |
| 301 | Not measured |
| 303 | 2.2^{ss} |
| 304 | 6.5 |
| 307 | 0.6^{ss} |

^{ss}denotes a steady state derived affinity constant.

TABLE 8
Change in binding affinity of WT G-CSF to design G-CSFR(Ig-
CRH)_{E} compared to WT:WT binding affinity determined by SPR

| | K_{D} fold change |
|---|---|
| WT:WT control | — |

Design G-CSFR(Ig-CRH)_{E}

| | |
|---|---|
| 9 | 8807^{ss} |
| 30 | 774 |
| 34 | 4258^{ss} |
| 35 | 19 |
| 130 | No binding |
| 134 | No binding |
| 401 | No binding |
| 402 | No binding |
| 300 | No binding |
| 301 | Not determined |
| 303 | No binding |
| 304 | No binding |
| 307 | No binding |

^{ss}denotes a steady state derived affinity constant

TABLE 9
Change in binding affinity of G-CSF_{E} to wild type G-CSFR(Ig-
CRH) compared to WT:WT binding affinity determined by SPR

| | K_{D} fold change |
|---|---|
| WT:WT control | — |

Design G-CSF_{E}

| | |
|---|---|
| 9 | 79 |
| 30 | 20 |
| 34 | 33 |
| 35 | 5.0 |
| 117 | 3.4 |
| 124 | No binding |
| 130 | No binding |
| 134 | 465 |
| 401 | No binding |
| 402 | No binding |
| 300 | No binding |
| 301 | Not determined |
| 303 | No binding |
| 304 | No binding |
| 307 | No binding |

### Example 7: Determination of the Thermal Stability of Design G-CSF_{E} Mutants

To determine the thermal stability of G-CSF_{E} and G-CSFR_{E} mutants in comparison to WT cytokine and receptor we performed differential scanning calorimetry (DSC).

### Methods

The thermal stability of variants was assessed by Differential Scanning Calorimetry (DSC) as follows: 950 mL of purified samples at concentrations of 1-2 mg/mL were used for DSC analysis with a Nano DSC (TA instruments, New Castle, DE). At the start of each run, buffer blank injections were performed for baseline stabilization. Each sample was scanned from 25 to 95° C. at a 60° C./hr rate, with 60 psi nitrogen pressure. The resulting thermograms were referenced and analyzed using NanoAnalyze software to determine melting temperature (Tm) as an indicator of thermal stability.

### Results

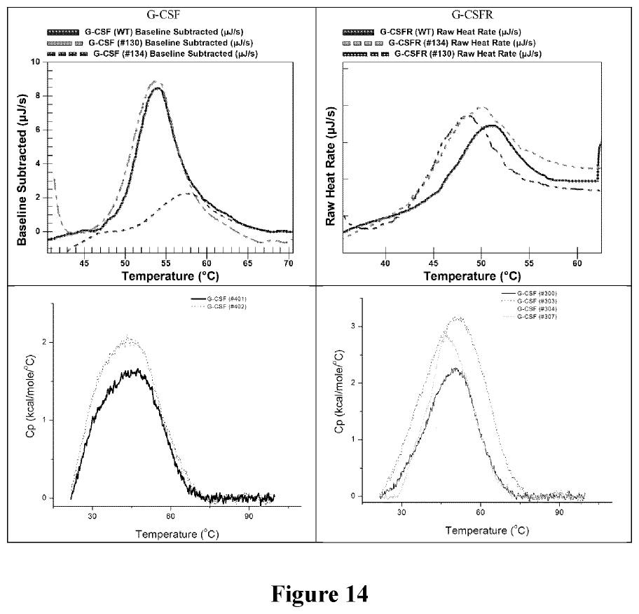

Thermal stability of the engineered variants is reported as the difference between the most prominent transition (highest enthalpy) of the engineered and the equivalent wild type molecule, measured under the same conditions and experimental set up. Measured WT GCSF Tm vary between 52.2 and 55.4° C. among independent experiments, while WT G-CSFR displays a Tm of 50.5° C. G-CSF_{E} mutants tested with the exception of designs #15 and 34 showed a Tm less than 5° C. different from the Tm for WT G-CSF (see, Table 10 and FIG. 14). All receptor mutants tested showed the same thermal stability as WT receptor (see, Table 10 and FIG. 14).

These results show that the variant G-CSF and receptors with the variant G-CSFR ECDs designs have similar thermal stability as wild type G-CSF and G-CSFR; and the site II and/or site III mutations do not disrupt thermal stability of either G-CSF or the G-CSFR ECD.

TABLE 10
Change in melting temperature (Tm) for design cytokine and
receptor mutants compared to wild type determined by DSC.

| | ΔTm (° C.) |
|---|---|
| GCSF_{E} design mutant | |
| WT | — |
| #15 | −9.9 |
| #34 | −6.1 |
| #35 | −3.5 |
| #106 | +7.5 |
| #130 | −1.8 |
| #134 | +2.0 |
| #135 | −0.8 |
| #300 | +1.0 |
| #301 | −3.2 |
| #303 | +4.2 |
| #304 | +2.7 |
| #307 | −3.8 |
| #401 | −2.5 |
| #402 | −3.8 |
| GCSFR (Ig-CRH)_{E} design mutant | |
| WT | — |
| #9 | +1.4 |
| #30 | +1.4 |
| #34 | +1.8 |
| #35 | +1.8 |
| #130* | −1.9 |
| #134 | −0.9 |
| #300 | Not measured |
| #301 | Not measured |
| #303 | Not measured |
| #304 | Not measured |
| #307 | Not measured |
| #401 | Not measured |
| #402 | Not measured |

*determined in 150 mM NaCl, 20 mM BisTris pH 6.5

### Example 8: Determination of the Monodispersity of G-CSF_{E} Mutants by UPLC-SEC

To determine the monodispersity of G-CSF_{E} mutants in comparison to WT G-CSF we analyzed mutants by UPLC-SEC.

### Methods

UPLC-SEC was performed on SEC purified protein samples using an Acquity BEH125 SEC column (4.6×150 mm, stainless steel, 1.7 μm particles) (Waters LTD, Mississauga, ON) set to 30° C. and mounted on an Agilent Technologies 1260 infinity II system with a PDA detector. Run times consisted of 7 minutes with a running buffer of 150 mM NaCl, 20 mM HEPES pH 8.0 or 150 mM NaCl, 20 mM BisTris pH 6.5 at a flow rate of 0.4 mL/min. Elution was monitored by UV absorbance in the range 210-500 nm, and chromatograms were extracted at 280 nm. Peak integration was performed using OpenLAB™ CDS ChemStation™ software.

### Results

WT G-CSF and design 34, 35 and 130 G-CSF_{E} mutants were 100% monodisperse (see, Table 11). Mutants 8, 9, 15, 117, 135 showed lower monodispersity between 65.3-79.5%. Design #134 cytokine showed 57.3% monodispersity at pH 8.0 which improved to 86.6% monodispersity at pH 6.5. The improvement in monodispersity at lower pH of the mobile phase could be due to a shift of pI, for example from a calculated pI of 5.41 for WT G-CSF to a calculated pI of 8.35 for design #134 G-CSF_{E}.

These results show that some of the variant G-CSF designs have 100% monodispersity as wild type G-CSF; indicating that a sub-set of the site II and/or site III mutations do not disrupt monodispersity of G-CSF; whereas other variant G-CSF designs led to lowered monodispersity that was increased at lower pH.

TABLE 11
Monodispersity of G-CSF_{E} design
mutants determined by UPLC-SEC.

| G-CSF_{E} Design Mutant | Monodispersity % |
|---|---|
| WT | 100.0 (96.7*) |
| 8 | 73.7 |
| 9 | 65.3 |
| 15 | 57.3 |
| 34 | 100.0 |
| 35 | 100.0 |
| 117 | 74.7* |
| 130 | 100.0* |
| 134 | 57.3 (86.6*) |
| 135 | 79.5* |
| 401 | 89.6* |

*in 150 mM NaCl, BisTris pH 6.5

### Example 9: Construction of Chimeric G-CSF Receptors with Intracellular IL-2 Receptor Signalling Domains

To investigate whether design G-CSF_{E} cytokine mutants are able to signal and cause immune cell proliferation through the design G-CSFR(Ig-CRH)_{E} receptor mutants, we constructed a single-chain chimeric G-CSF receptor using the G-CSFR ECD fused to the gp130 transmembrane (TM) domain and intracellular signaling domain (ICD), and an IL-2Rβ intracellular signaling domain (G-CSFR_{WT}-ICD_{gp130-IL-2Rβ}). We also utilized a chimeric G-CSFR that consists of two subunits designed to be co-expressed as a heterodimeric receptor: 1) The G-CSFR_{WT}-ICD_{IL-2Rβ} subunit consists of the G-CSFR ECD fused to the IL-2Rβ TM and ICD; and 2) The G-CSFR_{WT}-ICD_{γc} subunit consists of the G-CSFR ECD fused to the common gamma chain (γc, IL-2Rγ) TM and ICD.

### Methods

The single-chain chimeric receptor construct was designed to include the G-CSFR signal peptide and ECD, followed by the gp130 TM and partial ICD, and IL-2Rβ partial ICD (Table 12). The heterodimeric chimeric receptor construct was designed to include: 1) the G-CSFR signal peptide and ECD, followed by the IL-2Rβ TM and ICD (Table 13); and 2) the G-CSFR signal peptide and ECD, followed by the γc TM and ICD (Table 14). The chimeric receptor constructs were cloned into a lentiviral transfer plasmid and the construct sequences were verified by Sanger sequencing. The transfer plasmid and lentiviral packaging plasmids (psPAX2, pVSVG) were co-transfected into lentiviral packaging cell line HEK293T/17 cells (ATCC) as follows: Cells were plated overnight in DMEM containing 10% fetal bovine serum and penicillin/streptomycin, and medium changed 2-4 hours prior to transfection. Plasmid DNA and water were mixed in a polypropylene tube, and CaCl₂ (0.25M) was added dropwise. After a 2 to 5 minute incubation, the DNA was precipitated by mixing 1:1 with 2× HEPES-buffered saline (0.28M NaCl, 1.5 mM Na₂HPO₄, 0.1M HEPES). The precipitated DNA mixture was added onto the cells, which were incubated overnight at 37° C., 5% CO₂. The following day the HEK293T/17 medium was changed, and the cells were incubated for another 24 hours. The following morning, the cellular supernatant was collected from the plates, centrifuged briefly to remove debris, and filtered through a 0.45 μm filter. The supernatant was spun for 90 minutes at 25,000 rpm using a SW-32Ti rotor in a Beckman Optima L-XP Ultracentrifuge. The supernatant was removed, and the pellet was resuspended in a suitable volume of Opti-MEM medium. The viral titer was determined by adding serial dilutions of virus onto BAF3 cells (grown RPMI containing 10% fetal bovine serum, penicillin, streptomycin and 100 IU/ml hIL-2). 48-72 hours after transduction, the cells were incubated with an anti-human G-CSFR APC-conjugated antibody (1:50 dilution) and eBioscience™ Fixable Viability Dye eFluor™ 450 (1:1000 dilution) for 15 minutes at 4° C., washed, and analyzed on a Cytek Aurora or BD FACS Calibur flow cytometer. Using the estimated titer determined by this method, the 32D-IL-2Rβ cell line (grown in RPMI containing 10% fetal bovine serum, penicillin, streptomycin and 300 IU/ml hIL-2) was transduced with the lentiviral supernatant encoding the chimeric receptor construct at an MOI of 0.5. Transduction was performed by adding the relevant amount of viral supernatant to the cells, incubating for 24 hours, and replacing the cell medium. 3-4 days after transduction, we verified expression of the human G-CSFR by flow cytometry, as described above. Cells were expanded in G-CSF_{WT} for approximately 14-28 days before performing the BrdU assay.

32D-IL-2Rβ cells expanded in G-CSF_{WT} as described above were washed three times in PBS, and re-plated in fresh medium containing the relevant assay cytokine (no cytokine, hIL-2 (300 IU/ml), G-CSF_{WT} (30 ng/ml) or G-CSF_{E} (30 ng/ml) for 48 hours. The BrdU assay procedure followed the instruction manual for the BD Pharmingen™ APC BrdU Flow Kit (557892), with the following addition: cells were co-incubated with BrdU and eBioscience™ Fixable Viability Dye eFluor™ 450 (1:5000) for 30 minutes. Analytical flow cytometry was performed using a Cytek Aurora instrument.

### Results

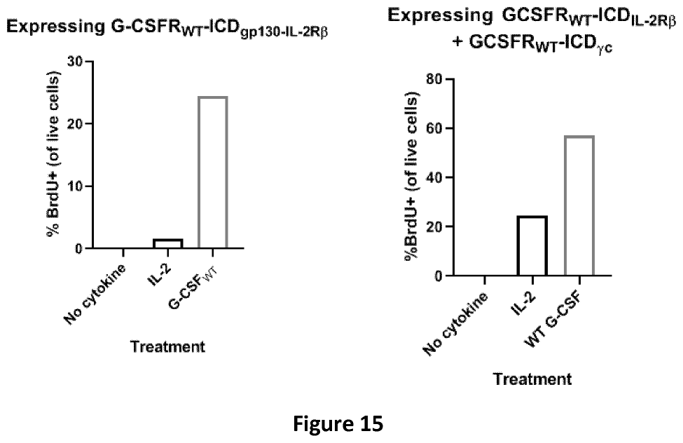

In a BrdU assay, cells transfected with the single-chain chimeric receptor construct G-CSFR_{WT}-ICD_{gp130-IL-2Rβ} or the heterodimeric receptor construct G-CSFR_{WT}-ICD_{IL-2Rβ} plus G-CSFR_{WT}-ICD_{γc} exhibited similar or superior proliferation in response to G-CSF_{WT} (30 ng/ml) compared to hIL-2 (300 IU/ml). Cells did not proliferate in the absence of cytokine (see FIG. 15).

These results show that, upon stimulation by G-CSF, single chain and heterodimeric chimeric receptor constructs can be activated to induce cell proliferation.

### Example 10: Proliferation of 32D-IL-2Rβ Cells Transduced with Design 137 G-CSFR_{E}-ICD_{IL-2} and Treated with Wild Type or Design G-CSF₁₃₇ Examined by BrdU

Site II/III combination designs that were sufficiently selective as judged by SPR or co-expression assays were tested in vitro for the ability to induce proliferation of 32D-IL-2Rβ cells.

### Methods

Point mutations for design 137 were introduced into the constructs described in Tables 12-14. Cloning and expression of the G-CSFR₁₃₇-ICD_{gp130-IL-2Rβ} (homodimer) or G-CSFR₁₃₇-ICD_{IL-2Rβ} plus G-CSFR₁₃₇-ICD_{γc} (heterodimer) constructs followed the same procedures as described above. Before performing the BrdU assay, cells were expanded in G-CSF₁₃₇ for approximately 14-28 days.

32D-IL-2Rβ cells expanded in G-CSF₁₃₇ were assayed for proliferation in G-CSF₁₃₇ (30 ng/ml), G-CSF_{WT} (30 ng/ml), hIL-2 (300 IU/ml) or no cytokine using the BrdU assay procedure described above.

### Results

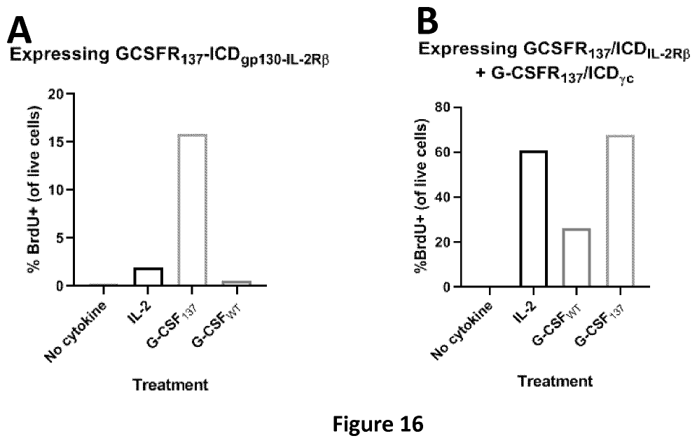

In a BrdU assay, cells transduced with the single-chain chimeric receptor construct G-CSFR₁₃₇-ICD_{gp130-IL-2Rβ} or the heterodimeric receptor construct G-CSFR₁₃₇-ICD_{IL-2Rβ} plus G-CSFR₁₃₇-ICD_{γc} exhibited similar or superior proliferation in G-CSF₁₃₇ (30 ng/ml) compared to hIL-2 (300 IU/ml). Cells did not proliferate in absence of cytokine and exhibited inferior proliferation in G-CSF_{WT} (30 ng/ml) (FIG. 16).

These results indicate that the variant G-CSF specifically activates the engineered receptor; and conversely, the engineered receptor is activated by the variant G-CSF but markedly less so than wild type G-CSF. Therefore, variant G-CSF can specifically activate chimeric receptors with variant G-CSFR ECD to specifically induce proliferation of cells expressing the chimeric receptors.

### Example 11: Proliferation of 32D-IL-2Rβ Cells Transduced with Wild-Type G-CSFR-ICD_{IL-2} and Treated with Wild Type or Design G-CSF_{E} Examined by BrdU

Site II/III combination designs that were able to restore paired signaling in 32D-IL-2R2Rβ cells were subsequently tested for their ability to induce proliferation of 32D-IL-2R2Rβ cells transduced with the single-chain chimeric receptor construct G-CSFR_{WT}-ICD_{gp130-IL-2Rβ} or the heterodimeric receptor construct G-CSFR_{WT}-ICD_{IL-2Rβ} plus G-CSFR_{WT}-ICD_{γc}.

### Methods

Cloning and expression of the G-CSFR_{WT}-ICD_{gp130-IL-2Rβ} (homodimer) or G-CSFR_{WT}-ICD_{IL-2Rβ} plus G-CSFR_{WT}-ICD_{γc} (heterodimer) constructs followed the same procedures as described above. Before performing the BrdU assay, cells were expanded in G-CSF_{WT} for approximately 14-28 days.

32D-IL-2Rβ cells expanded in G-CSF_{WT} were assayed for proliferation in G-CSF₁₃₇ (30 ng/ml), G-CSF_{WT} (30 ng/ml), hIL-2 (300 IU/ml) or no cytokine using the BrdU assay procedure described above.

### Results

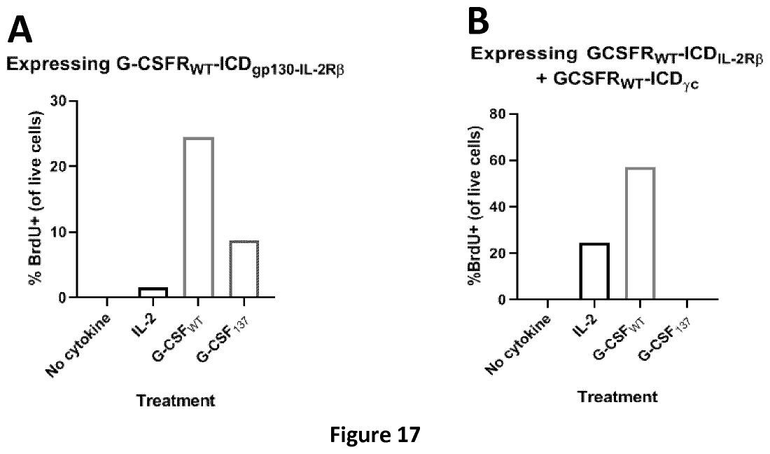

In a BrdU assay, cells transduced with the single-chain chimeric receptor construct G-CSFR_{WT}-ICD_{gp130-IL-2Rβ} or the heterodimeric receptor construct G-CSFR_{WT}-ICD_{IL-2Rβ} and G-CSFR_{WT}-ICD_{γc} exhibited inferior proliferation in G-CSF₁₃₇ (30 ng/ml) compared to G-CSF_{WT} (30 ng/ml). Cells did not proliferate in the absence of cytokine (see, FIG. 17).

These results indicate that the variant G-CSF does not efficiently bind wild type G-CSFR, and the variant G-CSF specifically activates the engineered receptor, but not wild type G-CSFR, to induce cell proliferation.

### Example 12: Signaling in 32D-IL-2Rβ Cells Transduced with WT or Design 137 G-CSFR_{E}-ICD_{IL-2} and Treated with Wild Type or Design G-CSF₁₃₇ Analyzed by Western Blot

Site II/III combination designs, that were able to restore proliferative signaling through the engineered cytokine-receptor complex in 32D-IL-2Rβ cells and did not significantly signal through binding G-CSF_{WT} or WT-GCSFR-ICD-IL2, were assessed by western blot for the ability to activate downstream signaling molecules in response to G-CSF_{WT} or G-CSF₁₃₇.

### Methods

Cloning and expression of the G-CSFR_{WT}-ICD_{gp130-IL-2Rβ} (homodimer) or G-CSFR_{WT}-ICD_{IL-2Rβ} plus G-CSFR_{WT}-ICD_{γc} (heterodimer) constructs followed the same procedures as described above. Cells were expanded in G-CSF₁₃₇ or G-CSF_{WT} for approximately 14-28 days before performing the western blot assay. Non-transduced cells were maintained in IL-2.

To perform western blots, cells were washed three times in PBS and rested in medium containing no cytokine for 16-20 hours. Cells were stimulated with no cytokine, IL-2 (300 IU/ml), G-CSF₁₃₇ (30 ng/ml) or G-CSF_{WT} (30 ng/ml) for 20 minutes at 37° C. Cells were washed once in a wash buffer containing 10 mM HEPES, pH 777.9, 1 mM MgCl₂, 0.05 mM EGTA, 0.5 mM EDTA, pH 8.0, 1 mM DTT, and 1× Pierce Protease and Phosphatase Inhibitor Mini Tablets (A32961). Cells were lysed in the wash buffer above, with the addition of 0.2% Igepal CA630 (Sigma), for 10 minutes on ice and centrifuged for 10 minutes at 13,000 rpm at 4° C., after which supernatant (cytoplasmic fraction) was collected. The pellet was resuspended and lysed in the above wash buffer with the addition of 0.42M NaCl and 20% glycerol. Cells were lysed for 30 minutes on ice, with frequent vortexing, and centrifuged for 20 minutes at 13,000 rpm at 4° C., after which the nuclear fraction (supernatant) was collected. The cytoplasmic and nuclear fractions were reduced (70° C.) for 10 minutes and run on a NuPAGE™ 4-12% Bis-Tris Protein Gel. The gels were transferred to nitrocellulose membrane (60 min at 20V in a Trans-Blot® SD Semi-Dry Transfer Cell), dried, and blocked for 1 hr in Odyssey® Blocking Buffer in TBS (927-50000). The blots were incubated with primary antibodies (1:1,000) overnight at 4° C. in Odyssey® Blocking Buffer in TBS containing 0.1% Tween20. The primary antibodies utilized were obtained from Cell Signaling Technologies: Phospho-Shc (Tyr239/240) Antibody #2434, Phospho-Akt (Ser473) (D9E) XP® Rabbit mAb #4060, Phospho-S6 Ribosomal Protein (Ser235/236) Antibody #2211, Phospho-p44/42 MAPK (Erk1/2) (Thr202/Tyr204) Antibody #9101, β-Actin (13E5) Rabbit mAb #4970, Phospho-Stat3 (Tyr705) (D3A7) XP® Rabbit mAb #9145, Phospho-Stat5 (Tyr694) (C11C5) Rabbit mAb #9359, and Histone H3 (96C10) Mouse mAb #3638. Blots were washed three times in TBS containing 0.1% Tween20 and incubated with secondary antibodies (1:10,000) in TBS buffer containing 0.1% Tween20 for 30-60 minutes at room temperature. The secondary antibodies obtained from Cell Signaling Technologies: Anti-mouse IgG (H+L) (DyLight™ 800 4× PEG Conjugate) #5257 and Anti-rabbit IgG (H+L) (DyLight™ 800 4× PEG Conjugate) #5151. Blots were washed and exposed on a LI-COR Odyssey imager.

### Results

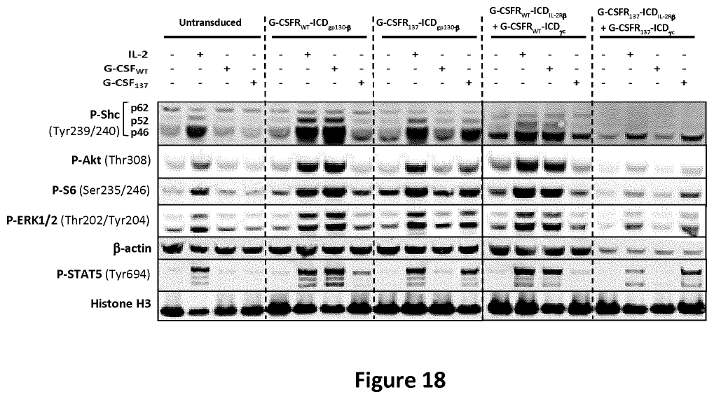

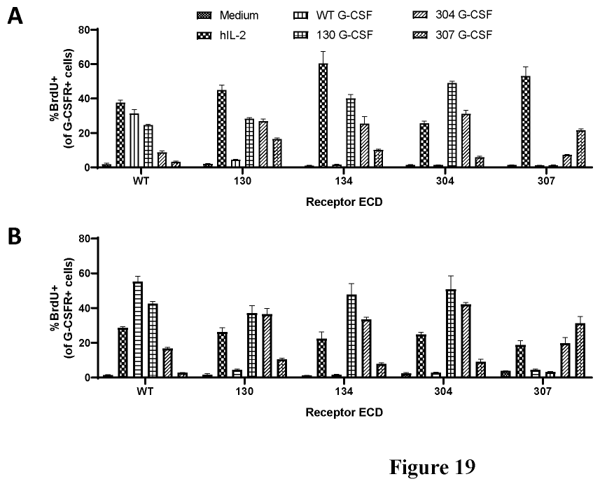

In non-transduced 32D-IL-2Rβ cells, we detected activated IL-2R-associated signaling molecules in response to stimulation with IL-2 only. We observed similar patterns of activated signaling molecules in response to IL-2 or G-CSF_{WT} in 32D-IL-2Rβ cells expressing either G-CSFR_{WT}-ICD_{gp130}-IL-2Rβ or G-CSFR_{WT}-ICD_{IL-2Rβ} plus G-CSFR_{WT}-ICD_{γc}. We did not observe activation of IL-2R-associated signaling molecules in response to G-CSF₁₃₇ stimulation of 32D-IL-2Rβ cells expressing G-CSFR_{WT}-ICD_{gp130-IL-2Rβ} or cells expressing G-CSFR_{WT}-ICD_{IL-2Rβ} plus G-CSFR_{WT}-ICD_{γc}. We observed similar patterns of activated signaling molecules in response to IL-2 or G-CSF₁₃₇ in 32D-IL-2Rβ cells expressing G-CSFR₁₃₇-ICD_{gp130-IL-2Rβ} or G-CSFR₁₃₇-ICD_{IL-2Rβ} plus G-CSFR₁₃₇-ICD_{γc}. We did not observe activation of IL-2R2R2R-associated signaling molecules in response to G-CSF_{WT} stimulation in 32D-IL-2Rβ cells expressing G-CSFR₁₃₇-ICD_{gp130-IL-2Rβ} or cells expressing G-CSFR₁₃₇-ICD_{IL-2Rβ} plus G-CSFR₁₃₇-ICD_{γc} (see FIG. 18).

These results demonstrate that variant G-CSF is capable of activating chimeric receptors expressing variant G-CSFR ECD to induce aspects of native cytokine signaling in cells expressing the chimeric receptors.

### Methods for Examples 13-30

Primary cells and cell lines: The lentiviral packaging cell line HEK293T/17 (ATCC) was cultured in DMEM containing 10% fetal bovine serum and penicillin/streptomycin. BAF3-IL-2Rβ cells were previously generated by stable transfection of the human IL-2Rβ subunit into the BAF3 cell line, and were grown in RPMI-1640 containing 10% fetal bovine serum, penicillin, streptomycin and 100 IU/ml human IL-2 (hIL-2) (PROLEUKIN®, Novartis Pharmaceuticals Canada). The 32D-IL-2Rβ cell line was previously generated by stable transfection of the human IL-2Rββ subunit into the 32D cell line, and was grown in RPMI-1640 containing 10% fetal bovine serum, penicillin, streptomycin and 300 IU/ml hIL-2, or other cytokines, as indicated. Human PBMC-derived T cells (Hemacare) were grown in TexMACS™ Medium (Milenyi Biotec, 130-097-196), containing 3% Human AB Serum (Sigma-Aldrich, H4522), and 300 IU/ml hIL-2, or other cytokines, as indicated. Human tumor-associated lymphocytes (TAL) were generated by culture of primary ascites samples for 14 days in T cell medium (a 50:50 mixture of the following: 1) RPMI-1640 containing 10% fetal bovine serum, 50 uM β-mercaptoethanol, 10 mM HEPES, 2 mM L-glutamine, penicillin, streptomycin; and 2) AIM V™ Medium (ThermoFisher, 12055083) containing a final concentration of 3000 IU/ml hIL-2. Following this high-dose IL-2 expansion, TAL were cultured in T cell medium containing 300 IU/ml hIL-2 or other cytokines, as indicated. The retroviral packaging cell line Platinum-E (Cell Biolabs, RV-101) was cultured in DMEM containing 10% FBS, penicillin/streptomycin, puromycin (1 mcg/ml), and blasticidin (10 mcg/ml).

Lentiviral production and transduction of 32D-IL-2Rβ cells: Chimeric receptor constructs were cloned into a lentiviral transfer plasmid and the resulting sequences were verified by Sanger sequencing. The transfer plasmid and lentiviral packaging plasmids were co-transfected into HEK293T/17 cells using the calcium phosphate transfection method as follows: Cells were plated overnight, and medium changed 2-4 hours prior to transfection. Plasmid DNA and water were mixed in a polypropylene tube, and CaCl₂ (0.25 M) was added dropwise. After a 2 to 5 minute incubation, the DNA was precipitated by mixing 1:1 with 2× HEPES-buffered saline (0.28M NaCl, 1.5 mM Na₂HPO₄, 0.1M HEPES). The precipitated DNA mixture was added onto the cells, which were incubated overnight at 37° C., 5% CO₂. The following day the HEK293T/17 medium was changed, and the cells were incubated for another 24 hours. The following morning, the cellular supernatant was collected from the plates, centrifuged briefly to remove debris, and the supernatant was filtered through a 0.45 micron filter. The supernatant was spun for 90 minutes at 25000 rpm with a SW-32Ti rotor in a Beckman Optima L-XP Ultracentrifuge. The supernatant was removed and the pellet was resuspended in a suitable volume of Opti-MEM medium. The viral titer was determined by adding serial dilutions of virus onto BAF3-IL-2Rβ cells. At 48-72 hours after transduction, the cells were incubated with an anti-human G-CSFR APC-conjugated antibody (1:50; Miltenyi Biotec, 130-097-308) and Fixable Viability Dye eFluor™ 450 (1:1000, eBioscience™, 65-0863-14) for 15 minutes at 4° C., washed, and analyzed on a Cytek Aurora or BD FACS Calibur flow cytometer. Using the estimated titer determined by this method, the 32D-IL-2Rβ cell line was transduced with the lentiviral supernatant encoding the chimeric receptor construct at a Multiplicity of Infection (MOI) of 0.5. Transduction was performed by adding the relevant amount of viral supernatant to the cells, incubating for 24 hours, and then replacing the medium. At 3-4 days after transduction, the expression of human G-CSFR was determined by flow cytometry, as described above.

Lentiviral transduction of human primary T cells: For transduction of PBMC-derived T cells and TAL, cells were thawed and plated in the presence of Human T Cell TransAct™ (Miltenyi Biotec, 130-111-160), according to the manufacturer's guidelines. 24 hours after activation, lentiviral supernatant was added, at MOI of 0.125-0.5. 48 hours after activation, the cells split into fresh medium, to remove residual virus and activation reagent. Two to four days after transduction, the transduction efficiency was determined by flow cytometry, as described above. For experiments in which the transduction efficiency of CD4+ and CD8+ fractions was determined separately, antibodies against human G-CSFR, CD4 (1:50, Alexa Fluor® 700 conjugate, BioLegend, 300526), CD8 (1:50, PerCP conjugate, BioLegend, 301030), CD3 (1:50, Brilliant Violet 510™ conjugate, BioLegend, 300448) and CD56 (1:50, Brilliant Violet 711™ conjugate, BioLegend, 318336) were utilized, along with Fixable Viability Dye eFluor™ 450 (1:1000).

Human T cell and 32D-IL-2Rβ expansion assays: Human primary T cells or 32-IL-2Rβ cells expressing the indicated chimeric receptor constructs, generated above, were washed three times in PBS and re-plated in fresh medium, or had their medium gradually changed, as indicated. Complete medium was changed to contain either wild-type human G-CSF (generated in-house or NEUPOGEN®, Amgen Canada), mutant G-CSF (generated in-house), hIL-2, or no cytokine. Every 3-5 days, cell viability and density were determined by Trypan Blue exclusion, and fold expansion was calculated relative to the starting cell number. G-CSFR expression was assessed by flow cytometry as described above.

CD4+ and CD8+ human TAL expansion assay: To examine the expansion of the CD4+ and CD8+ fractions of TAL, ex vivo ascites samples were thawed, and the CD4+ and CD8+ fractions were enriched using the Human CD4⁺ T Cell Isolation Kit (Miltenyi Biotec, 130-096-533) and Human CD8⁺ T Cell Isolation Kit (Miltenyi Biotec, 130-096-495), respectively. After expansion in cytokine-containing medium, the immunophenotype of the cells was assessed by flow cytometry, utilizing antibodies against human G-CSFR, CD4 (1:50, Alexa Fluor® 700 conjugate, BioLegend, 300526), CD8 (1:50, PerCP conjugate, BioLegend, 301030), CD3 (1:50, Brilliant Violet 510™ conjugate, BioLegend, 300448) and CD56 (1:50, Brilliant Violet 711™ conjugate, BioLegend, 318336), along with Fixable Viability Dye eFluor™ 450 (1:1000).

Primary human T cell immunophenotyping assay: After expansion in cytokine-containing medium, the immunophenotype of T cells was assessed by flow cytometry, utilizing antibodies against human G-CSFR, CD4 (1:100, Alexa Fluor® 700 conjugate, BioLegend, 300526 or PE conjugate, eBioscience™, 12-0048-42, or Brilliant Violet 570™ conjugate, Biolegend, 317445), CD8 (1:100, PerCP conjugate, BioLegend, 301030), CD3 (1:100, Brilliant Violet 510™ or Brilliant Violet 750™ conjugate, BioLegend, 300448 or 344845), CD56 (1:100, Brilliant Violet 711™ conjugate, BioLegend, 318336), CCR7 (1:50, APC/Fire™ 750 conjugate, Biolegend, 353246), CD62L (1:33, PE/Dazzle™ 594 conjugate, Biolegend, 304842), CD45RA (1:33, FITC conjugate, Biolegend, 304148), CD45RO (1:25, PerCP-eFluor® 710 conjugate, eBioscience™, 46-0457-42), CD95 (1:33, PE-Cyanine7 conjugate, eBioscience™, 25-0959-42), along with Fixable Viability Dye eFluor™ 450 or 5106 (1:1000).

Retroviral transduction: The pMIG transfer plasmid (plasmid #9044, Addgene) was altered by restriction endonuclease cloning to remove the IRES-GFP (BglII to PacI sites), and introduce annealed primers encoding a custom multiple cloning site. The chimeric receptor constructs were cloned into the customized transfer plasmid and the resulting sequences were verified by Sanger sequencing. The transfer plasmid was transfected into Platinum-E cells using the calcium phosphate transfection method, as described above. 24 hours after transfection the medium was changed to 5 ml fresh complete medium. 48 hours after transfection the cellular supernatant was collected from the plates and filtered through a 0.45 micron filter. Hexadimethrine bromide (1.6 mcg/ml, Sigma-Aldrich) and murine IL-2 (2 ng/ml, Peprotech) were added to the supernatant. This purified retroviral supernatant was used to transduce murine lymphocytes as described below.

48 hours prior to collection of retroviral supernatant, 24-well adherent plates were coated with unconjugated anti-murine CD3 (5 mcg/ml, BD Biosciences, 553058) and anti-murine CD28 (1 mcg/ml, BD Biosciences, 553294) antibodies, diluted in PBS, and stored at 4 degrees Celsius. 24 hours prior to collection of retroviral supernatant, C57Bl/6J mice (generated in-house) were euthanized under an approved Animal Use Protocol administered by the University of Victoria Animal Care Committee. Spleens were harvested and murine T cells were isolated as follows: Spleens were manually dissociated and filtered through a 100 micron filter. Red blood cells were lysed by incubation in ACK lysis buffer (Gibco, A1049201) for five minutes at room temperature, followed by one wash in serum-containing medium. CD8a-positive or Pan-T cells were isolated using specific bead-based isolation kits (Miltenyi Biotec, 130-104-075 or 130-095-130, respectively). Cells were added to plates coated with anti-CD3- and anti-CD28-antibodies in murine T cell expansion medium (RPMI-1640 containing 10% FBS, penicillin/streptomycin, 0.05 mM β-mercaptoethanol, and 2 ng/ml murine IL-2 (Peprotech, 212-12)) or 300 IU/mL human IL-2 (Proleukin), and incubated at 37 degrees Celsius, 5% CO₂ for 24 hours. On the day of transduction, approximately half of the medium was replaced with retroviral supernatant, generated above. Cells were spinfected with the retroviral supernatant at 1000 g, for 90 minutes, at 30 degrees Celsius. The plates were returned to the incubator for 0-4 hours, and then approximately half of the medium was replaced with fresh T cell expansion medium. The retroviral transduction was repeated 24 hours later, as described above, for a total of two transductions. 24 hours after the final transduction, the T cells were split into 6-well plates and removed from antibody stimulation.

48-72 hours after transduction, the transduction efficiency was assessed by flow cytometry to detect human G-CSFR, CD4 (Alexa Fluor 532 conjugate, eBioscience™, 58-0042-82), CD8a (PerCP-eFluor 710 conjugate, eBioscience™, 46-0081-82) and Fixable Viability Dye eFluor™ 450 (1:1000 dilution), as described above.

BrdU incorporation assay: Human primary T cells, 32D-IL-2Rβ cells, or murine primary T cells, generated as described above, were washed three times in PBS, and re-plated in fresh medium containing the relevant assay cytokine: no cytokine, hIL-2 (300 IU/ml), wildtype or engineered G-CSF (at concentrations indicated in individual experiments) for 48 hours. The BrdU assay procedure followed the instruction manual for the BD Pharmingen™ APC BrdU Flow Kit (BD Biosciences, 557892), with the following additions: Cells were co-incubated with BrdU and Fixable Viability Dye eFluor™ 450 (1:5000) for 30 minutes to 4 hours at 37 degrees Celsius. Flow cytometry was performed using a Cytek Aurora instrument. To specifically assess the proliferation of murine T cells expressing chimeric receptors, additional staining for human G-CSFR (1:20 dilution), CD4 (1:50 dilution) and CD8 (1:50 dilution) was performed for 15 minutes on ice, prior to fixation.

Western blots: Human primary T cells, 32D-IL-2Rβ cells, or murine primary T cells, generated as described above, were washed three times in PBS and rested in medium containing no cytokine for 16-20 hours. Cells were stimulated with no cytokine, IL-2 (300 IU/ml), wildtype G-CSF (at concentrations indicated in individual experiments), or G-CSF₁₃₇ (30 ng/ml) for 20 minutes at 37 degrees Celsius. Cells were washed once in a buffer containing 10 mM HEPES, pH7.9, 1 mM MgCl₂, 0.05 mM EGTA, 0.5 mM EDTA, pH 8.0, 1 mM DTT, and 1× Pierce Protease and Phosphatase Inhibitor Mini Tablets (A32961). Cells were lysed in the wash buffer above, with the addition of 0.2% NP-40 (Sigma) for 10 minutes on ice. The lysate was centrifuged for 10 minutes at 13000 rpm at 4 degrees Celsius, and the supernatant (cytoplasmic fraction) was collected. The pellet (containing nuclear proteins) was resuspended in the wash buffer above, with the addition of 0.42M NaCl and 20% glycerol. Nuclei were incubated for 30 minutes on ice, with frequent vortexing, and the supernatant (nuclear fraction) was collected after centrifuging for 20 minutes at 13,000 rpm at 4 degrees Celsius. The cytoplasmic and nuclear fractions were reduced (70 degrees Celsius) for 10 minutes and run on a NuPAGE™ 4-12% Bis-Tris Protein Gel. The gels were transferred to nitrocellulose membrane (60 min at 20V in a Trans-Blot® SD Semi-Dry Transfer Cell), dried, and blocked for 1 hr in Odyssey® Blocking Buffer in TBS (927-50000). The blots were incubated with primary antibodies (1:1000) overnight at 4 degrees Celsius in Odyssey® Blocking Buffer in TBS containing 0.1% Tween20. The primary antibodies utilized were obtained from Cell Signaling Technologies: Phospho-JAK1 (Tyr1034/1035) (D7N4Z) Rabbit mAb #74129, Phospho-JAK2 (Tyr1007/1008) #3771, Phospho-JAK3 (Tyr980/981) (D44E3) Rabbit mAb #5031, Phospho-p70 S6 Kinase (Thr421/Ser424) Antibody #9204, Phospho-Shc (Tyr239/240) Antibody #2434, Phospho-Akt (Ser473) (D9E) XP® Rabbit mAb #4060, Phospho-S6 Ribosomal Protein (Ser235/236) Antibody #2211, Phospho-p44/42 MAPK (Erk1/2) (Thr202/Tyr204) Antibody #9101, β-Actin (13E5) Rabbit mAb #4970, Phospho-STAT1 (Tyr70l) (58D6) Rabbit mAb #9167, Phospho-STAT3 (Tyr705) (D3A7) XP® Rabbit mAb #9145, Phospho-STAT4 (Tyr693) Antibody #5267, Phospho-STAT5 (Tyr694) (C11C5) Rabbit mAb #9359, and Histone H3 (96C10) Mouse mAb #3638. Blots were washed three times in TBS containing 0.1% Tween20 and incubated with secondary antibodies (1:10,000) in TBS buffer containing 0.1% Tween20 for 30-60 minutes at room temperature. The secondary antibodies obtained from Cell Signaling Technologies were Anti-mouse IgG (H+L) (DyLight™ 800 4× PEG Conjugate) #5257 and Anti-rabbit IgG (H+L) (DyLight™ 800 4× PEG Conjugate) #5151. Blots were washed and exposed on a LI-COR Odyssey imager.

Flow cytometry to detect phosphorylated proteins: Human primary T cells, 32D-IL-2Rβ cells, or murine primary T cells, generated as described above, were washed three times in PBS and rested in medium containing no cytokine for 16-20 hours. Cells were stimulated with no cytokine, IL-2 (300 IU/ml) or wild-type G-CSF (100 ng/ml) for 20 minutes at 37 degrees Celsius, in the presence of Fixable Viability Dye eFluor™ 450 (1:1000), and anti-G-CSFR (1:20), anti-CD4 (1:50) and anti-CD8a (1:50), as indicated. Cells were pelleted and fixed with BD Phosflow™ Fix Buffer I (BD Biosciences, 557870) for 15 minutes at room temperature. Cells were washed, then permeabilized using BD Phosflow™ Perm Buffer III (BD Biosciences, 558050) on ice for 15 minutes. Cells were washed twice and resuspended in buffer containing 20 ul of BD Phosflow™ PE Mouse Anti-Stat3 (pY705) (BD Biosciences, 612569) or PE Mouse IgG2a, κ Isotype Control (BD Biosciences, 558595). Cells were washed and flow cytometry was performed using a Cytek Aurora instrument.

### Example 13: Expansion of Human T Cells Expressing G2R-1, G-CSFR/IL-2R_{B} Subunit Alone, the Myc-Tagged G-CSFR/Gamma-C Subunit Alone, or the Full-Length G-CSFR

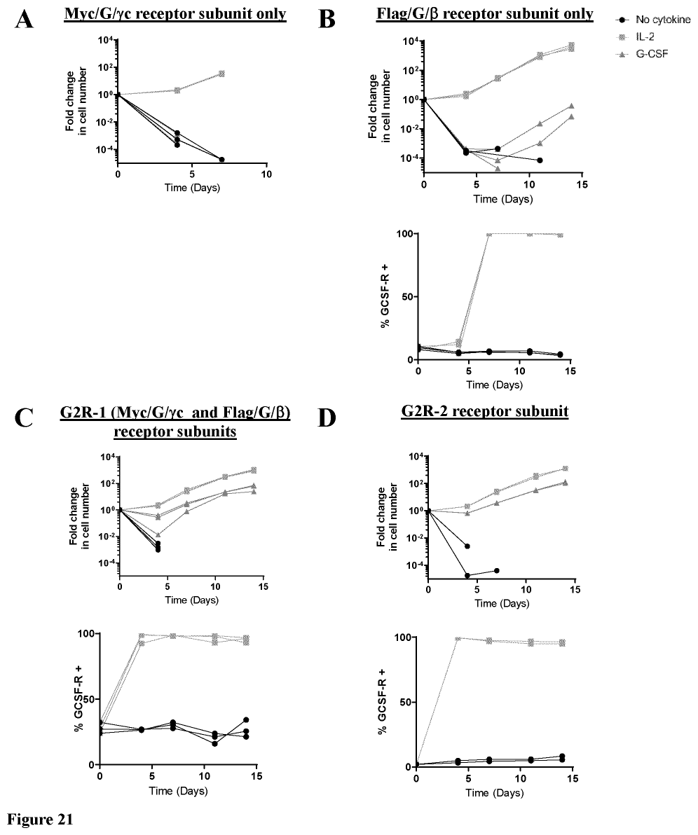

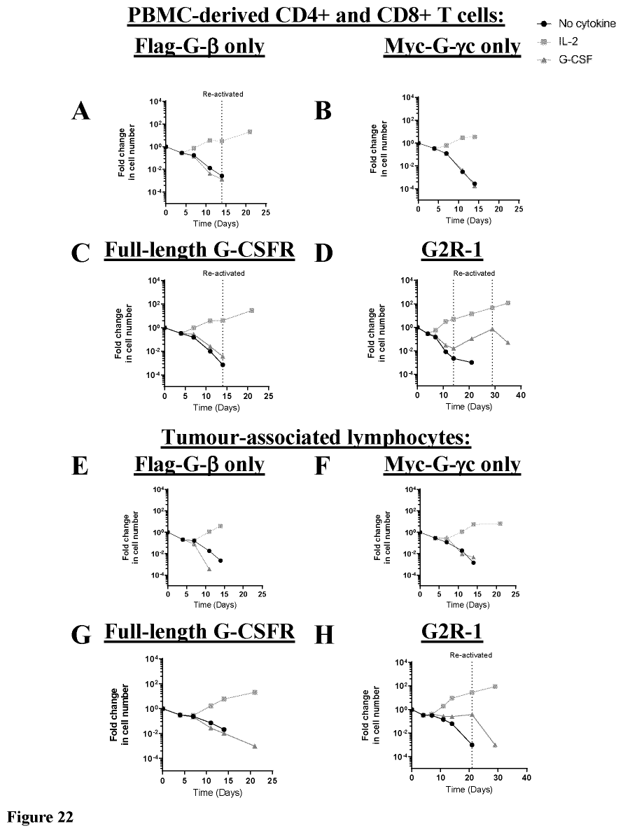

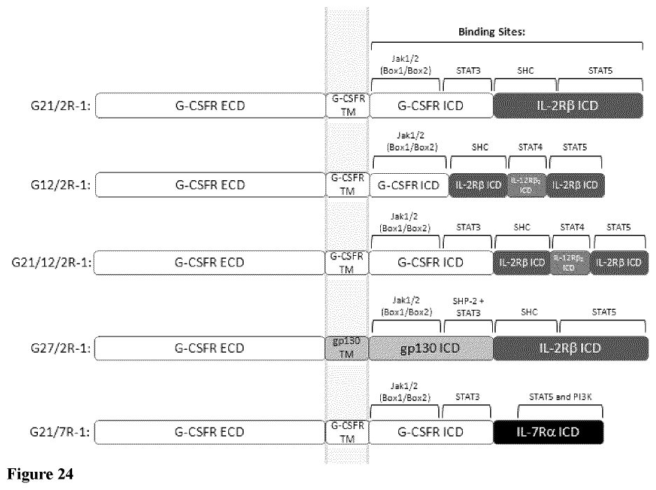

PBMC-derived T cells or tumor-associated lymphocytes (TAL) were transduced with lentiviruses encoding the chimeric receptor constructs shown in FIGS. 20, 24 and 24, and cells were washed and re-plated in the indicated cytokine. Cells were counted every 3-4 days. G/γc was tagged at its N-terminus with a Myc epitope (Myc/G/γc), and G/IL-2Rβ was tagged at its N-terminus with a Flag epitope (Flag/G/IL-2Rβ); these epitope tags aid detection by flow cytometry and do not impact the function of the receptors. As expected, all T cell cultures showed proliferation in response to the positive control cytokine, IL-2 (300 IU/ml). After stimulation with G-CSF (100 ng/ml), proliferation was observed only for PMBC-derived T cells and TAL expressing the G2R-1 chimeric cytokine receptor (FIG. 22). Note that the lentiviral transduction efficiency was less than 100% such that less than 100% of T cells expressed the indicated chimeric cytokine receptors, which likely accounts for the lower rate of proliferation mediated by G2R-1 relative to IL-2. Similarly, increased proliferation was observed in 32D-IL-2Rβ cells (stably expressing the human IL-2Rβ subunit) expressing G-CSFR chimeric receptor subunits G2R-1 and G2R-2 and stimulated with G-CSF (FIG. 21). In contrast to T cells, 32D-IL-2Rβ cells expressing the G/IL-2Rβ chimeric receptor subunit alone proliferated in response G-CSF (FIG. 21); G-CSF-induced proliferation was not seen in 32D-IL-2Rβ cells expressing the G/γc chimeric receptor subunit alone (FIG. 21).

These results show that G-CSF is capable of stimulating proliferation and viability of PMBC-derived T cells and TALs expressing the G2R-1 chimeric receptors and of 32D-IL-2Rβ cells expressing the G/IL-2Rβ, G2R-1 and G2R-2 chimeric receptors.

### Example 14: G-CSFR ECD is Expressed on the Surface of Cells Transduced with G/IL-2Rβ, G2R-1 and G2R-2

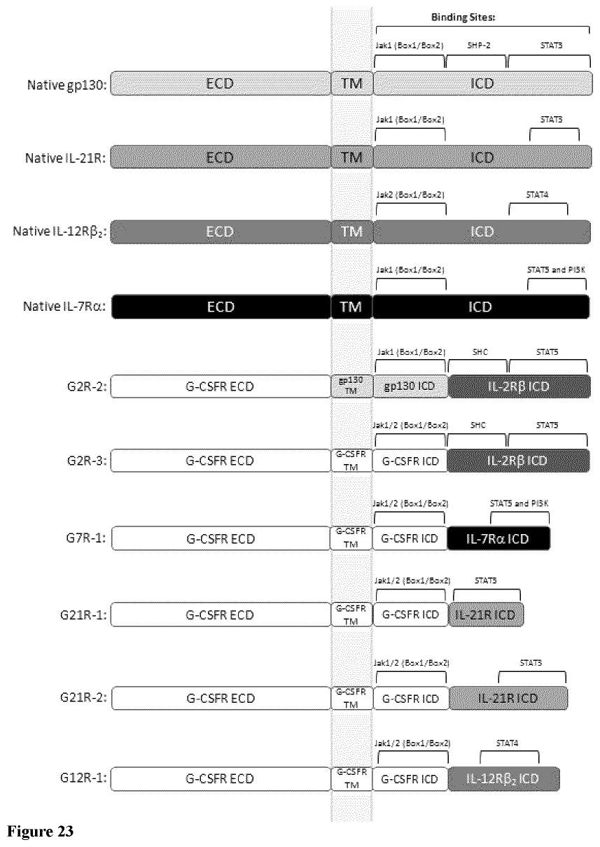

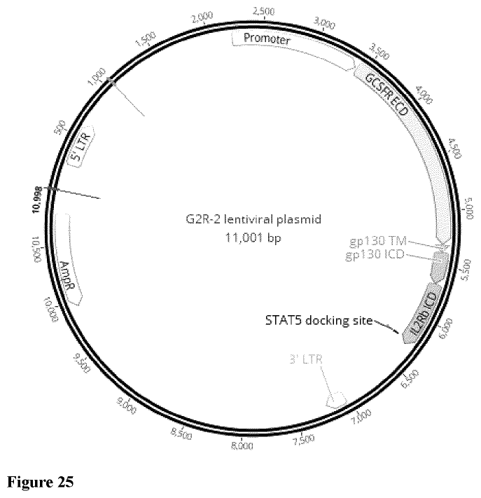

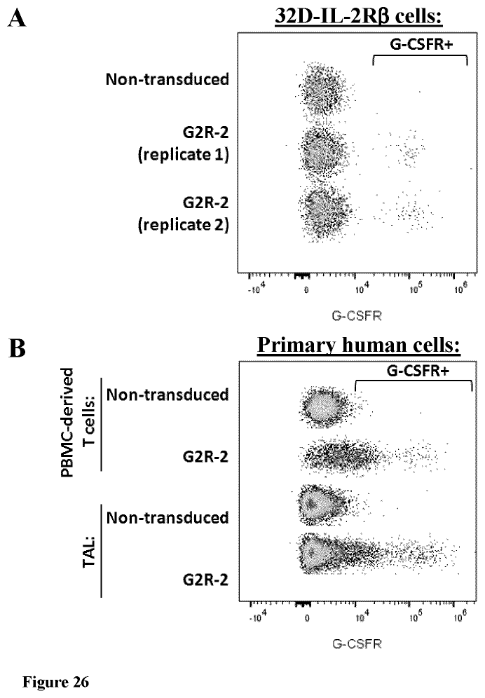

Flow cytometry was performed on the 32D-IL-2Rβ cell line, PBMC-derived human T cells and human tumor-associated lymphocytes after transduction with a lentiviral vector encoding the G2R-2 chimeric cytokine receptor (shown schematically in FIGS. 23 and 25) to determine if the cells express the G-CSFR ECD on the cell surface. G-CSFR positive cells were detected in all transduced cell types (FIG. 26). In a separate experiment, 32D-IL-2Rβ cells expressing the G/IL-2Rβ, G2R-1 and G2R-2 chimeric receptors were positive for the G-CSFR ECD by flow cytometry (lower panels in FIG. 21B-D).

These results indicate that the G/IL-2Rβ, G2R-1 and G2R-2 chimeric receptors are expressed on the cell surface.

### Example 15: Expansion of Cells Expressing G2R-2 Compared to Non-Transduced Cells

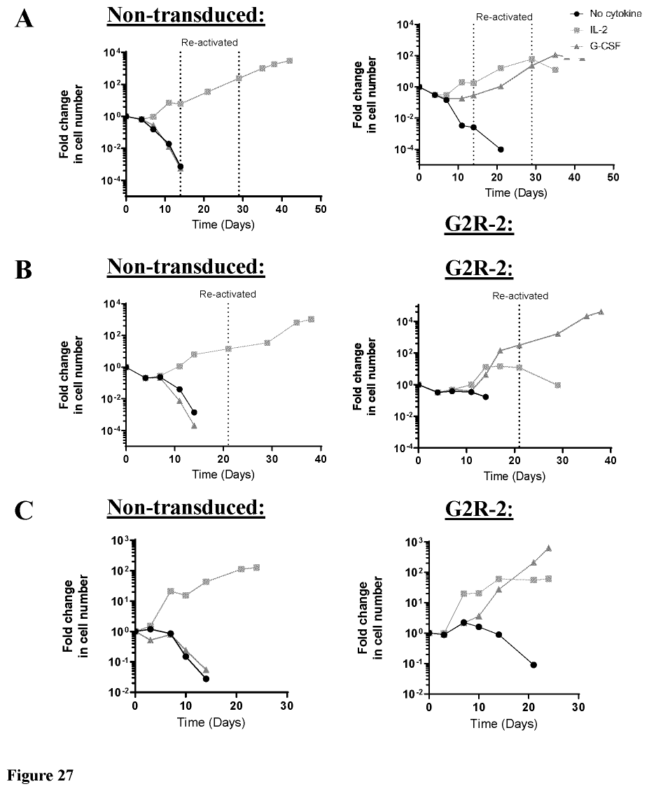

Human PBMC-derived T cells and human tumor-associated lymphocytes were lentivirally transduced with the G2R-2 receptor construct (FIGS. 23 and 25), washed, and re-plated with the indicated cytokine. In some experiments, T cells were also re-activated periodically by stimulation with TransAct. Live cells were counted every 3-4 days. Proliferation of the PMBC-derived T cells (FIG. 27A) and tumor-associated lymphocytes (two independent experiments in FIG. 27B,C) was observed after stimulation with G-CSF (100 ng/ml) in cells expressing the G2R-2 chimeric receptor but not in non-transduced cells.

These results show that G-CSF-induced activation of the G2R-2 chimeric receptor is sufficient to induce proliferation and viability of immune cells.

### Example 16: Expansion and Immunophenotype of CD4- or CD8-Selected Human Tumor-Associated Lymphocytes Expressing G2R-2 Compared to Non-Transduced Cells

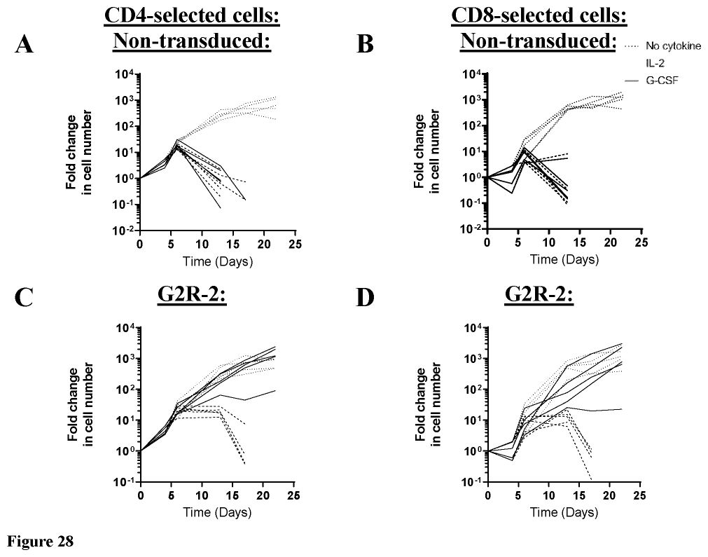

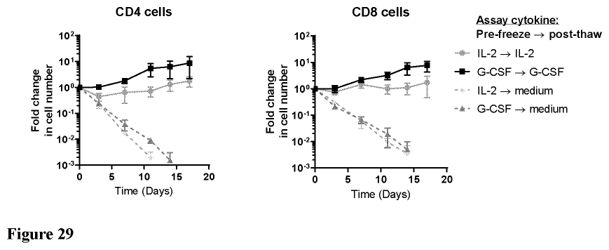

CD4-selected and CD8-selected human T cells were transduced with lentiviral vector encoding G2R-2 (FIG. 25), or left non-transduced where indicated. Cells were washed and re-plated with the indicated cytokine and counted every 3-4 days. Proliferation of CD4- or CD8-selected TALs expressing G2R-2 was observed after stimulation with G-CSF (100 ng/ml) or IL-2 (300 IU/ml), but not in the absence of added cytokine (medium alone) (FIGS. 28 and 29). In FIG. 28, each line represents results from one of 5 patient samples.

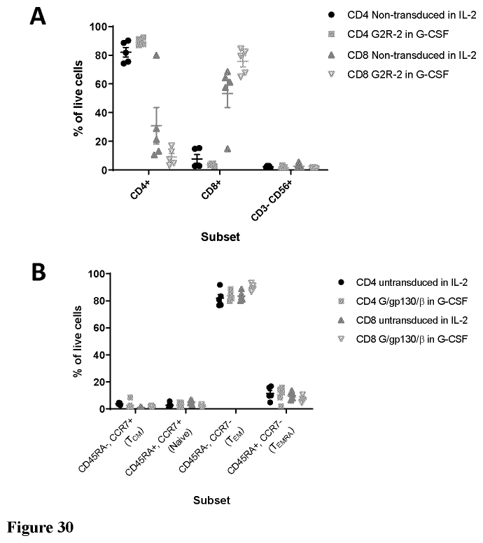

Immunophenotyping by flow cytometry demonstrated that T cells cultured in G-CSF or IL-2 retained their CD4+ or CD8+ identity (FIG. 30A), lacked an NK cell phenotype (CD3−CD56+) (FIG. 30A), and exhibited a CD45RA−CCR7− T effector memory (T_{EM}) phenotype under these culture conditions (FIG. 30B).

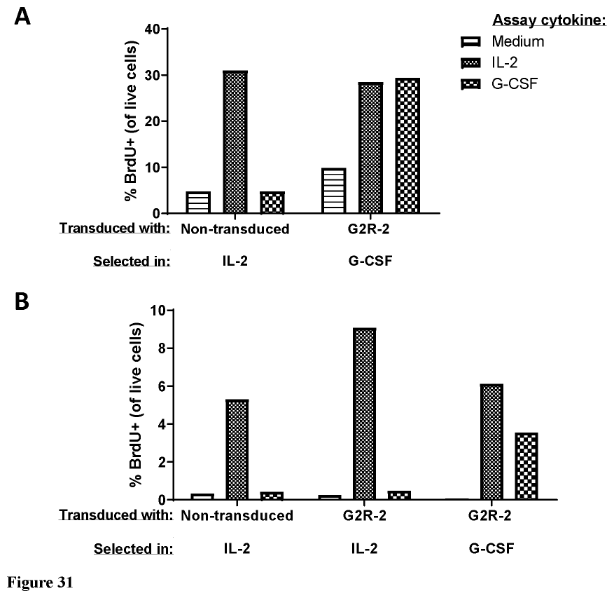

BrdU assays were performed to confirm increased cell cycle progression of T cells expressing G2R-2 upon stimulation with G-CSF (FIG. 31). T cells were selected by culture in IL-2 or G-CSF, as indicated, prior to the assay. Both tumor-associated lymphocytes (FIG. 31A) and PBMC-derived T cells (FIG. 31B) were assessed.

These results show that G-CSF can selectively activate cell cycle progression and long-term expansion of primary human TALs by activation of the chimeric cytokine receptor G2R-2. These results also indicate that activation of the G2R-2 chimeric receptor by homodimer formation is sufficient to activate cytokine-like signaling and proliferation in TALs. Furthermore, TALs expressing G2R-2 remain cytokine dependent in that they undergo cell death upon withdrawal of G-CSF, similar to the response to IL-2 withdrawal. TALs cultured in G-CSF maintain a similar immunophenotype as TALs cultured in IL-2.

### Example 17: Primary Murine T Cells Expressing G2R-2 Proliferate in Response to G-CSF

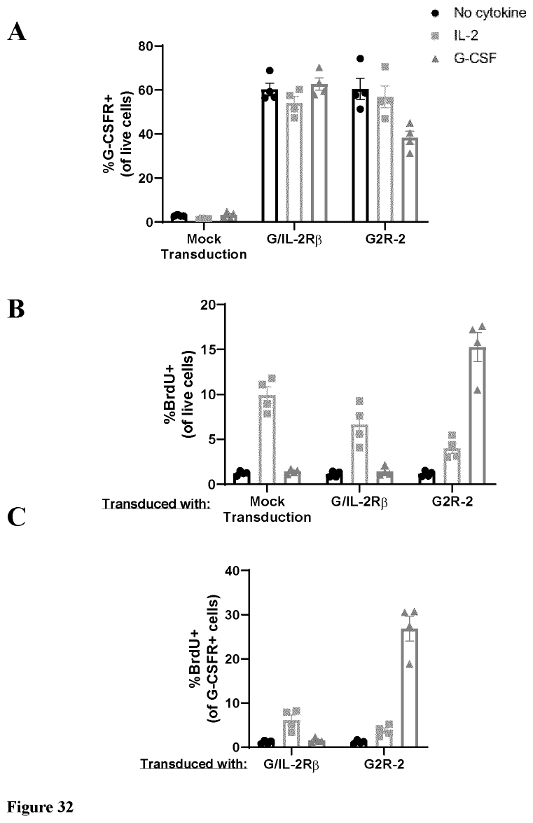

BrdU incorporation assays were performed to assess proliferation of primary murine T cells expressing G2R-2 or the single-chain G/IL-2Rβ (a component of G2R-1) versus mock-transduced cells upon stimulation with G-CSF. All cells were expanded in IL-2 for 3 days prior to assay. Cell surface expression of G2R-2 or G/IL-2Rβ was confirmed by flow cytometry (FIG. 32A). As indicated, cells were then plated in IL-2 (300 IU/ml), wildtype G-CSF (100 ng/ml) or no cytokine. Increased cell cycle progression after stimulation with G-CSF was observed in the cells expressing G2R-2 above that of non-transduced cells or cells expressing the single-chain G/IL-2Rβ (FIG. 32B,C). Panels B and C show results for all live cells or G-CSFR+ cells, respectively.

These results indicate that, in response to G-CSF-induced homodimerization, the G2R-2 chimeric receptor is more efficient than the single chain G/IL-2Rβ receptor to activate cytokine-like signaling and proliferation in murine T cells.

### Example 18: Activation of Cytokine-Associated Intracellular Signaling Events in of Human Primary T Cells Expressing G2R-2 in Response to G-CSF or IL-2

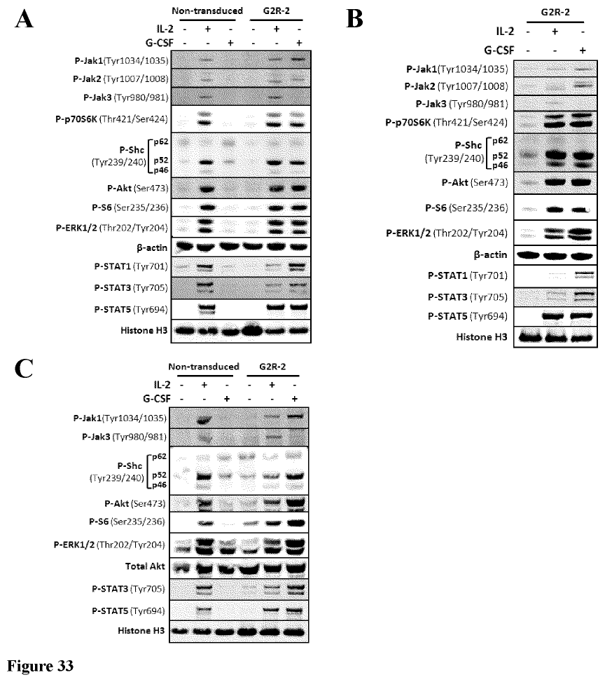

To confirm that the chimeric cytokine receptors were indeed capable of activating cytokine signaling similar to that of IL-2, the ability of the cytokine receptors to activate various signaling molecules was assessed. Tumor-associated lymphocytes and PBMC-derived T cells expressing G2R-2 were previously expanded in G-CSF, while non-transduced cells were previously expanded in IL-2. The cells were washed and then stimulated with IL-2 (300 IU/ml), wildtype G-CSF (100 ng/ml), or no cytokine, and western blots were performed on the cell lysates using antibodies directed against the indicated signaling molecules (FIG. 33). Panels A and B show results for TAL, and panel C for PBMC-derived T cells. T cells expressing G2R-2 activated IL-2-related signaling molecules upon stimulation with G-CSF to a similar extent as seen after IL-2 stimulation of non-transduced cells or transduced cells, with the expected exception that G-CSF induced JAK2 phosphorylation whereas IL-2 induced JAK3 phosphorylation.

These results confirm that the G2R-2 chimeric receptor is capable of activating IL-2 receptor-like cytokine receptor signaling upon stimulation with G-CSF.

### Example 19: Cytokine Signaling is Activated in Response to G-CSF in Murine Primary T Cells Expressing G2R-2

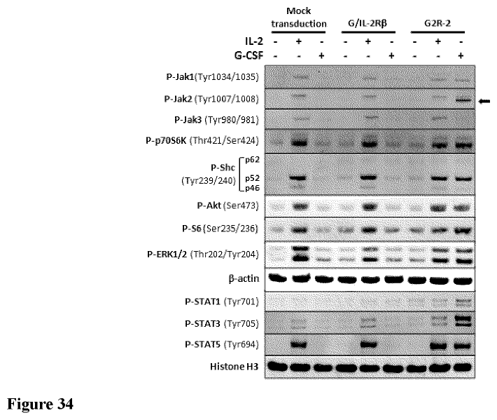

To assess whether the chimeric cytokine receptor G2R-2 or the single-chain G/IL-2Rβ (from G2R-1) were capable of activating cytokine signaling, the ability of these cytokine receptors to activate various signaling molecules was assessed by western blot of cell lysates of murine primary T cells expressing G2R-2 or G/IL-2Rβ versus mock-transduced cells. All cells were expanded in IL-2 for 3 days prior to assay. Cells were then washed and stimulated with IL-2 (300 IU/ml), wildtype G-CSF (100 ng/ml) or no cytokine. The cells expressing G2R-2 activated IL-2-related signaling molecules upon stimulation with G-CSF to a similar extent as seen after IL-2 stimulation of non-transduced cells or transduced cells, with the expected exception that G-CSF induced JAK2 phosphorylation whereas IL-2 induced JAK3 phosphorylation (FIG. 34). In contrast, G/IL-2Rβ did not activate cytokine signaling upon exposure to G-CSF.

These results confirm in primary murine T cells that the G2R-2 chimeric receptor is capable of activating IL-2 receptor-like cytokine receptor signaling by homodimerization upon G-CSF stimulation, whereas the single-chain G/IL-2Rβ alone is not capable of activating cytokine signaling by homodimerization in response to G-CSF.

### Example 20: Expression of Chimeric Receptors Leads to Proliferation of 32D-IL-2Rβ Cells and Primary Murine T Cells after Stimulation with an Orthogonal G-CSF

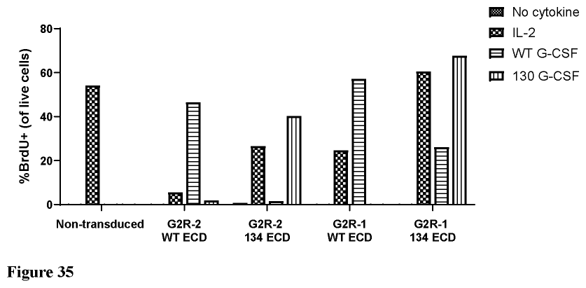

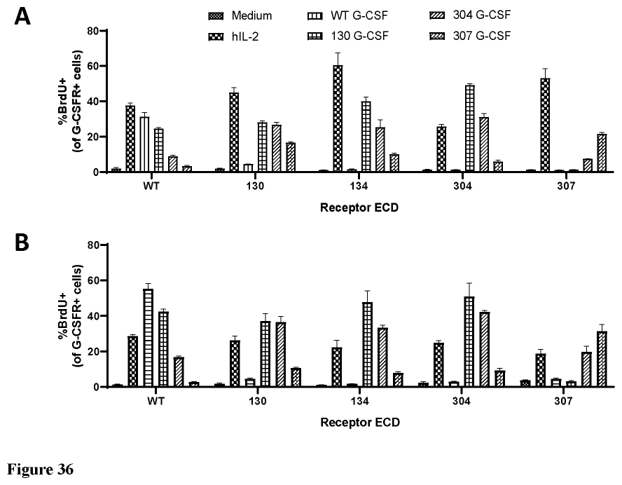

To determine if cells expressing chimeric cytokine receptors could be selectively activated in response to an orthogonal version of G-CSF, 32D-IL-2Rβ cells or primary murine T cells were transduced with the chimeric receptors G2R-1 and G2R-2 that comprises the wild-type G-CSFR ECD (G2R-1 WT ECD, G2R-2 WT ECD) and chimeric receptors G2R-1 and G2R-2 that comprises the G-CSFR ECD which harbors the amino acid substitutions R41E, R141E and R167D (G2R-1 134 ECD, G2R-2 134 ECD). Cells were stimulated with either IL-2, wild type G-CSF or the orthogonal G-CSF (130 G-CSF) capable of binding to G2R-1 134 ECD and G2R-2 134 ECD, but with significantly reduced binding to wild-type G-CSFR. BrdU incorporation assays were performed to assess the ability of the cells to promote cell cycle progression upon cytokine stimulation (FIG. 35). 32D-IL-2Rβ cells expressing G2R-2 134 ECD demonstrated cell cycle progression upon stimulation with 130 G-CSF (harboring amino acid substitutions E46R, L108K, and D112R; 30 ng/ml), but did not undergo cell cycle progression upon stimulation with wild-type G-CSF (30 ng/ml). The orthogonal nature of engineered cytokine:receptor ECD pairs was further demonstrated by stimulating primary murine T cells in a “criss-cross” proliferation assay, where cells expressing G2R-3 (FIG. 23) with the WT, 130, 134, 304 or 307 ECD were stimulated with WT, 130, 304 or 307 cytokine (100 ng/ml) (FIG. 36). The 130 ECD harbors the amino acid substitutions: R41E, and R167D. The 304 ECD harbors the amino acid substitutions: R41E, E93K and R167D; whereas the 304 cytokine harbors the amino acid substitutions: E46R, L108K, D112R and R147E. The 307 ECD harbors the amino acid substitutions: R41E, D197K, D200K and R288E; whereas the 307 cytokine harbors the amino acid substitutions: S12E, K16D, E19K and E46R. Panels A and B in FIG. 36 represent experimental replicates.

These results demonstrate that cells expressing an orthogonal chimeric cytokine receptor are capable of selective activation and cell cycle progression upon stimulation with an orthogonal G-CSF CSF.

### Example 21: Intracellular Signaling is Activated in 32D-IL2Rβ Cells and Primary Human T Cells Expressing Orthogonal Chimeric Cytokine Receptors and Stimulated with an Orthogonal G-CSF

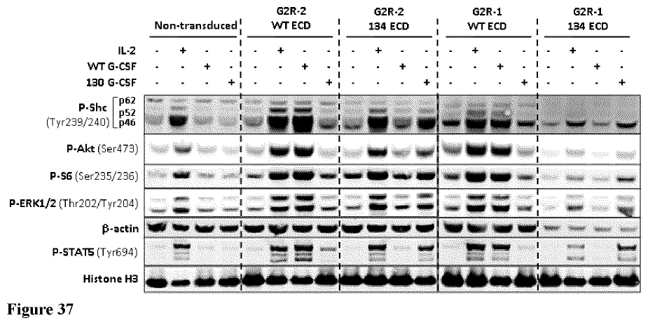

To determine if cells expressing chimeric cytokine receptors could selectively activate intracellular cytokine signaling events in response to an orthogonal version of G-CSF, 32D-IL-2Rβ cells were transduced with the chimeric receptors G2R-1 and G2R-2 that comprises the wild-type G-CSFR ECD (G2R-1 WT ECD and G2R-2 WT ECD) and chimeric receptors G2R-1 and G2R-2 that comprises the G-CSFR ECD which harbors the amino acid substitutions R41E, R141E and R167D (G2R-1 134 ECD, G2R-2 134 ECD). Cells were stimulated with either IL-2 (300 IU/ml), wild type G-CSF (30 ng/ml) or the orthogonal G-CSF (130 G-CSF—E46R_L108K_D112R; 30 ng/ml) capable of binding to G2R-1 134 ECD, G2R-2 134 ECD, but with significantly reduced binding to wild-type G-CSFR. Western blots were performed on cell lysates to assess the ability of the cells to activate cytokine signaling upon exposure to cytokines (FIG. 37). Cells expressing G2R-2 134 ECD showed evidence of cytokine signaling upon stimulation with 130 G-CSF but not wild-type G-CSF. Furthermore, cells expressing G2R-2 WT ECD were not able to activate cytokine signaling upon stimulation with 130 G-CSF.

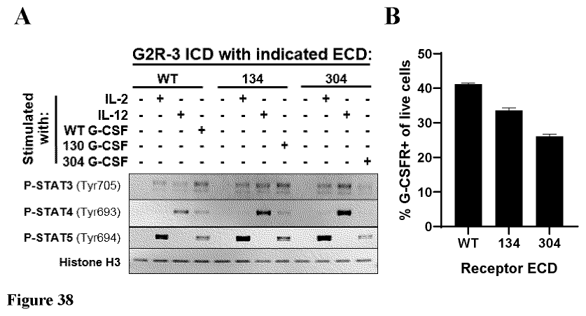

The orthogonal nature of engineered cytokine: receptor pairs was further demonstrated by subjecting primary murine T cells to western blot analysis, where cells expressing G2R-3 with the WT, 134, or 304 ECD (R41E_E93K_R167D) stimulated with WT, 130, or 304 G-CSF (E46R_L108K_D112R_R147E; 100 ng/ml) and the indicated signaling events were measured (FIG. 38A). IL-2 (300 IU/ml) and IL-12 (10 ng/ml) served as control cytokines. Cells expressing G2R-3 WT ECD showed evidence of cytokine signaling upon stimulation with IL-2, IL-12 or WT G-CSF. Cells expressing G2R-3 134 ECD showed evidence of cytokine signaling upon stimulation with IL-2, IL-12 or 130 G-CSF. Cells expressing G2R-3 304 ECD showed evidence of cytokine signaling upon stimulation with IL-2, IL-12 or 304 G-CSF.

Cell surface expression of the three ECD variants of G2R-3 was confirmed by flow cytometry (FIG. 38B).

These results demonstrate that cells expressing an orthogonal chimeric cytokine receptor are capable of selective activation of intracellular cytokine signaling events upon stimulation with an orthogonal G-CSF.

### Example 22: Expression of G2R-3 Leads to Expansion, Cell Cycle Progression and Cytokine-Related Intracellular Signaling and Immunophenotype in Primary Human T Cells

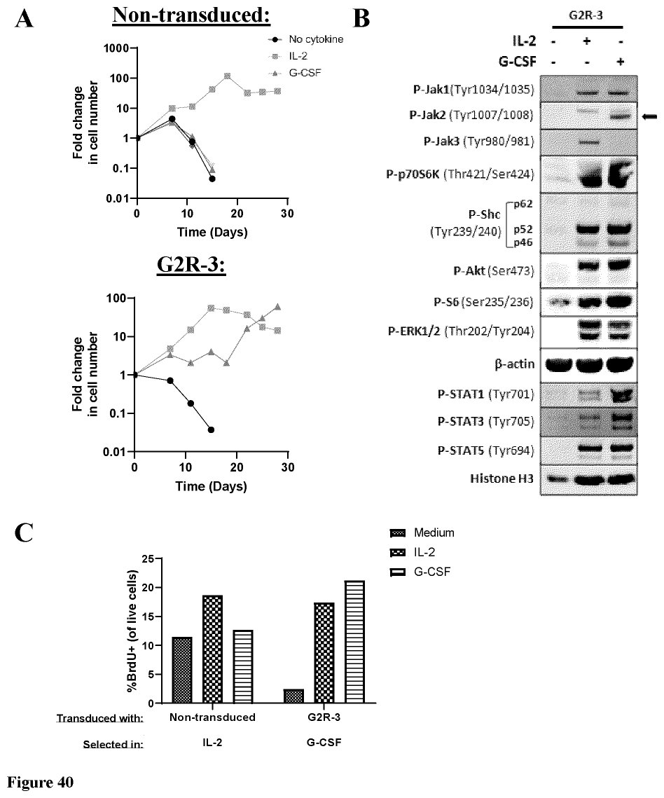

To determine whether the G2R-3 chimeric receptor can promote cytokine signaling-related events upon stimulation with G-CSF in primary human T cells, TALs were transduced with a lentiviral vector encoding G2R-3. T-cell expansion assays were performed to test the proliferation of cells upon stimulation with IL-2 (300 IU/ml), wildtype G-CSF (100 ng/ml) or no cytokine. Live cells were counted every 3-4 days. In contrast to their non-transduced counterparts, primary TALs expressing G2R-3 expanded in culture in response to G-CSF (FIG. 40A). To determine whether cytokine signaling events were activated upon stimulation with G-CSF, western blots were performed on cell lysates to assess intracellular signaling. Cells were harvested from the expansion assay, washed, and stimulated with IL-2 (300 IU/ml) or wildtype G-CSF (100 ng/ml). Primary TALs expressing G2R-3 demonstrated IL-2-related signaling events in response to G-CSF, with the expected exception that G-CSF induced JAK2 phosphorylation whereas IL-2 induced JAK3 phosphorylation (FIG. 40B).

BrdU incorporation assays were performed to assess cell cycle progression upon stimulation with G-CSF. Cells were harvested from the expansion assay, washed, and re-plated in IL-2 (300 IU/ml), wildtype G-CSF (100 ng/ml) or no cytokine. Primary TALs expressing G2R-3 demonstrated cell cycle progression in response to G-CSF (FIG. 40C).

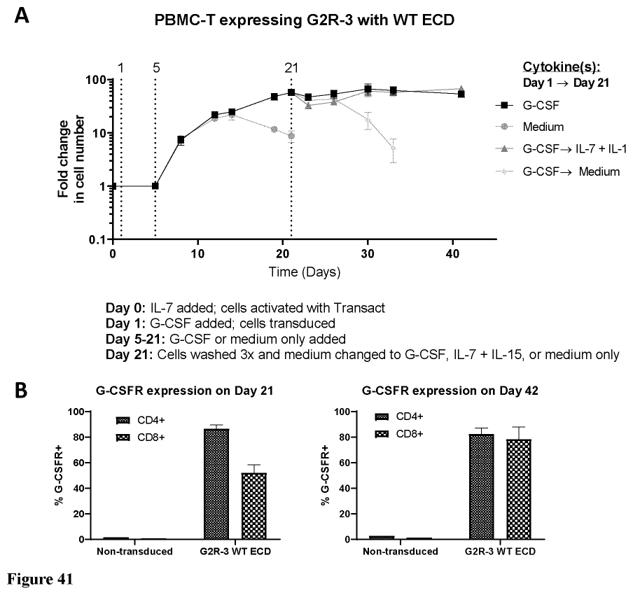

G-CSF-induced expansion of cells expressing G2R-3 was also demonstrated using primary PBMC-derived human T cells (FIG. 41). Cells expressing G2R-3 WT ECD expanded in response to WT G-CSF but not medium alone (FIG. 41A). To demonstrate the continued dependence of cells on exogenous cytokine, on Day 21 of culture, cells from the G-CSF-expanded condition were washed and re-plated in WT G-CSF (100 ng/mL), IL-7 (20 ng/mL)+IL-15 (20 ng/mL), or medium only. Only cells re-plated in the presence of G-CSF or IL-7+IL-15 remained viable over time.

Expression of G-CSFR ECD, as assessed by flow cytometry, remained stable on both CD4+ and CD8+ T cells between days 21-42 of expansion (FIG. 41B).

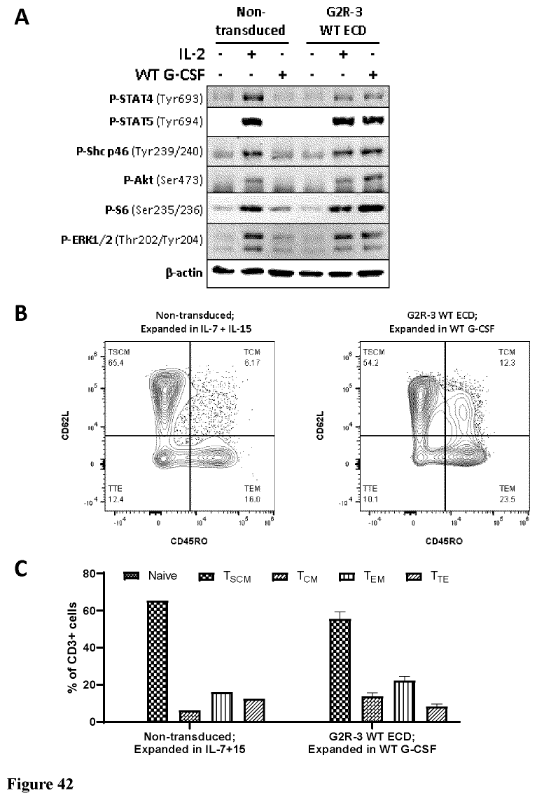

By western blot, primary PBMC-derived T cells expressing G2R-3 demonstrated IL-2-related signaling events in response to G-CSF (FIG. 42A). Flow cytometry-based immunophenotyping was performed on primary PBMC-derived T cells expanded for 42 days in WT G-CSF versus IL-7+IL-15. Cells expressing G2R-3 WT ECD and cultured in G-CSF retained a similar phenotype to non-transduced cells cultured in IL-7+IL-15, with primarily a CD62L+, CD45RO+ phenotype, which is indicative of a stem cell-like memory T cell phenotype (T_{SCM}) (FIG. 42B, C). Likewise, the fractions of central memory (T_{CM}), effector memory (T_{EM}), and terminally differentiated (T_{TE}) T cells were similar.

These results confirm that the G2R-3 chimeric cytokine receptor is capable of activating cytokine signaling events and promoting cell cycle progression and expansion in primary cells. The immunophenotype of T cells expressing G2R-3 and expanded long-term in G-CSF is similar to non-transduced cells expanded in IL-7+IL-15.

### Example 23: Orthogonal G-CSF Induces Expansion and Proliferation in Primary Human T Cells Expressing G2R-3 with Orthogonal ECD

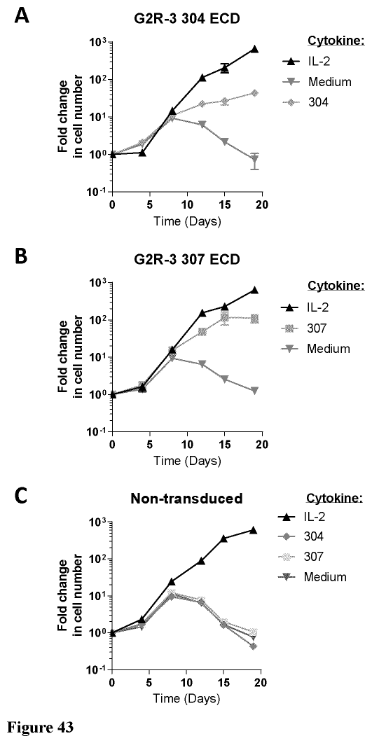

We assessed whether the chimeric cytokine receptor G2R-3 with 304 (R41E_E93K_R167D) or 307 (R41E_D197K_D200K_R288E) ECD was capable of inducing proliferation and expansion in response to stimulation with the orthogonal ligand 130, 304 or 307 G-CSF, primary PBMC-derived human T cells were transduced with lentiviral vectors encoding GR-3 304 ECD or G2R-3 307 (R41E_D197K_D200K_R288E) ECD. T-cell growth assays were performed to assess the fold expansion of cells when cultured with IL-2 (300 IU/ml), 304 G-CSF (100 ng/ml), 307 G-CSF (100 ng/ml) or no cytokine. Live cells were counted every 3-4 days. T cells expressing G2R-3 304 ECD expanded in culture in response to IL-2 or 304 G-CSF (FIG. 43A). T cells expressing G2R-3 307 ECD expanded in culture in response to IL-2 or 307 G-CSF (FIG. 43B). Non-transduced T cells only expanded in response to IL-2 (FIG. 43C).

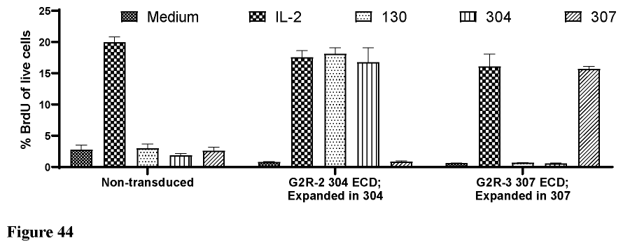

BrdU incorporation assays were performed to assess cell cycle progression upon stimulation with 130, 304 and 307 G-CSF in a criss-cross design. Cells were harvested from the expansion assay, washed, and re-plated in IL-2 (300 IU/ml), 130 G-CSF (100 ng/ml), 304 G-CSF (100 ng/ml), 307 G-CSF (100 ng/ml) or no cytokine. Primary human T cells expressing G2R-3 304 ECD demonstrated cell cycle progression in response to 130 or 304 G-CSF, but not in response to 307 G-CSF (FIG. 44). T cells expressing G2R-3 307 ECD demonstrated cell cycle progression in response to 307 G-CSF, but not in response to 130 or 304 G-CSF. All T cells demonstrated cell cycle progression in response to IL-2.

The results show that the chimeric receptors G2R-3 304 ECD and G2R-3 307 ECD are capable of inducing selective cell cycle progression and expansion of primary human CD4+ and CD8⁺ T cells upon stimulation with orthogonal 304 or 307 G-CSF, respectively. Furthermore, 130 G-CSF can stimulate proliferation of cells expressing G2R-3 304 ECD, but not G2R-3 307 ECD.

### Example 24: G-CSFR ECD is Expressed on the Surface of Primary Human Tumor-Associated Lymphocytes (TAL) Transduced with G21R-1, G21R-2, G12R-1 and G2R-3 Chimeric Receptor Constructs

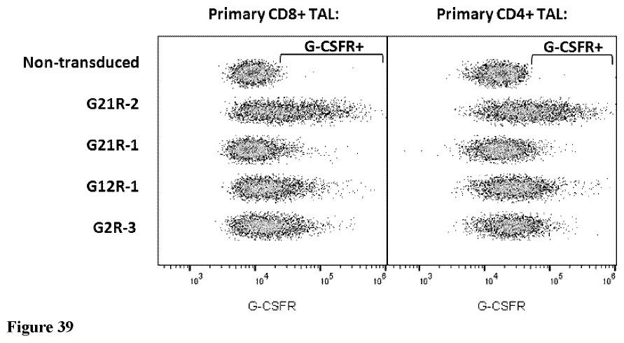

To assess whether chimeric cytokine receptor constructs can be expressed on the surface of primary human tumor-associated lymphocytes (TAL), TAL were transduced with lentiviral vectors encoding the G21R-1, G21R-2, G12R-1 and G2R-3 chimeric receptors, and the cells were tested by flow cytometry for G-CSFR ECD expression on the cell surface (FIG. 39). For all four chimeric cytokine receptor designs, G-CSFR ECD positive cells were detected.

These results demonstrate that the G21R-1, G21R-2, G12R-1 and G2R-3 chimeric receptors are capable of being expressed on the surface of primary cells. These results also indicate that G-CSFR ECD chimeric receptor designs are expressed on the surface of primary cells.

### Example 25: G-CSFR ECD is Expressed on the Surface of Primary Murine T Cells Transduced with G12R-1 and G21R-1 Chimeric Receptor Constructs

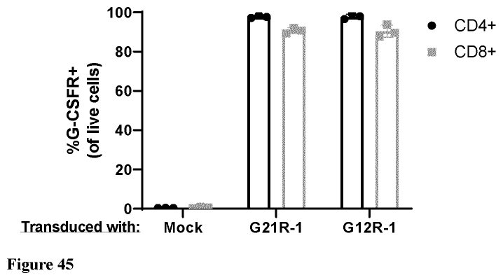

To determine if the G12R-1 and G21R-1 chimeric receptors are capable of being expressed on the surface of primary T cells, primary murine T cells were transduced with retroviral vectors encoding the G12R-1 and G21R-1 chimeric receptors and analyzed by flow cytometry (FIG. 45).

The results show that G-CSFR ECD is expressed on the surface of primary murine CD4+ and CD8+ T cells transduced with retroviral vectors encoding G12R-1 and G21R-1.

### Example 26: G-CSF Induces Cytokine Signaling Events in Primary PBMC-Derived Human T Cells Expressing G21R-1 or G21R-2

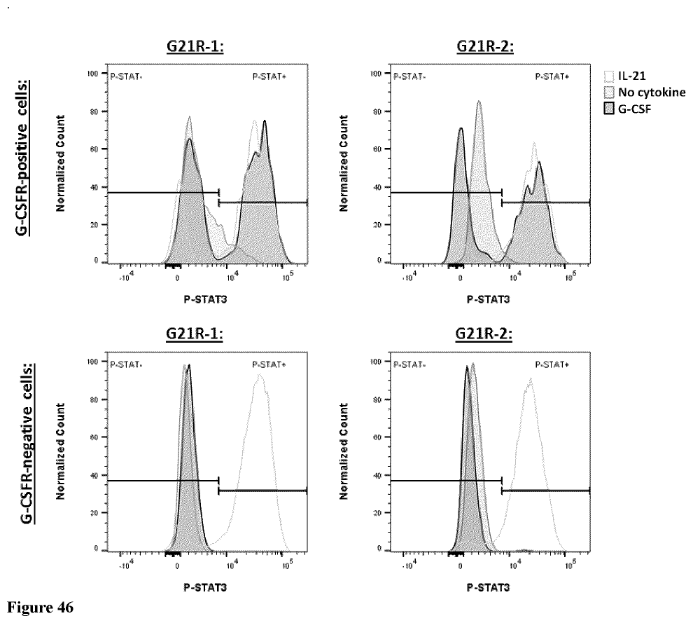

To determine whether the G21R-1 and G21R-2 constructs are capable of inducing cytokine signaling events in primary cells, primary PBMC-derived human T cells were transduced with lentiviral vectors encoding G21R-1 or G21R-2 chimeric cytokine receptors. Cells were subjected to intracellular staining with phospho-STAT3 (p-STAT3) specific antibody and assessed by flow cytometry to determine the extent of STAT3 phosphorylation, a measure of STAT3 activation (FIG. 46). Upon stimulation with G-CSF (100 ng/ml), the number of cells expressing phosphorylated STAT3 increased among the subset of G-CSFR positive cells transduced with either G21R-1 or G21R-2. In contrast, the G-CSFR negative (i.e. non-expressing) cells did not exhibit an increase in phosphorylated STAT3 upon stimulation with G-CSF but did upon stimulation with IL-21.

These results demonstrate that the G21R-1 and G21R-2 chimeric receptors are capable of activating IL-21-related cytokine signaling events upon stimulation with G-CSF in primary human T cells.

### Example 27: G-CSF Induces Intracellular Signaling Events in Primary Murine T Cells Expressing G21R-1 or G-12R-1

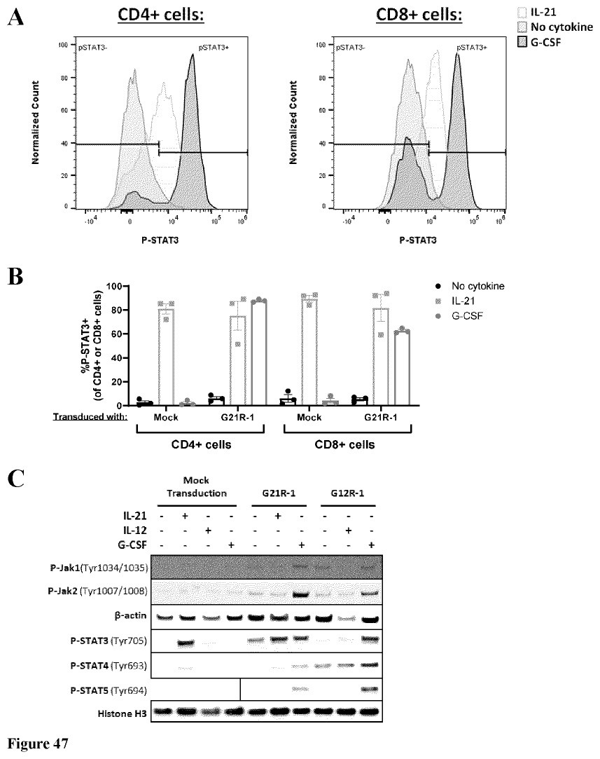

To determine if the chimeric cytokine receptor G21R-1 was capable of activating cytokine signaling events, primary murine T cells were transduced with a retroviral vector encoding G21R-1 and assessed by flow cytometry to detect phosphorylated STAT3 upon stimulation with G-CSF. Live cells were gated on CD8 or CD4, and the percentage of cells staining positive for phospho-STAT3 after stimulation with no cytokine, IL-21 (1 ng/ml) or G-CSF (100 ng/ml) was determined for the CD8 and CD4 cell populations. Upon stimulation with G-CSF, cells expressing G21R-1 (but not non-transduced cells) showed increased amounts of phosphorylated STAT3 (FIGS. 47A and 47B). Western blots were performed to assess intracellular cytokine signaling in cells expressing G21R-1 or G12R-1 upon stimulation with G-CSF. As expected, cells expressing G21R-1 and stimulated with G-CSF showed increased phosphorylation of STAT3, with minor increases in phospho-STAT4 and phospho-STAT5 (FIG. 47C). Also as expected, in cells expressing G12R-1, strong phosphorylation of STAT4 was seen in response to G-CSF. G-CSF did not induce any signaling events in mock transduced cells. (Note that in the G12R-1 group, the positive control (hIL-12 10 ng/ml) did not appear to induce any signaling events; this may be due to poor binding of human IL-12 to the murine IL-12R.)

The results show that G21R-1 and G12R-1 are capable of inducing cytokine signaling events in primary murine T cells upon stimulation with G-CSF.

### Example 28: G-CSF Induces Proliferation and Intracellular Signaling Events in Primary Murine T Cells Expressing G2R-2, G2R-3, G7R-1, G21/7R-1, G27/2R-1, G21/2R-1, G12/2R-1 or G21/12/2R-1

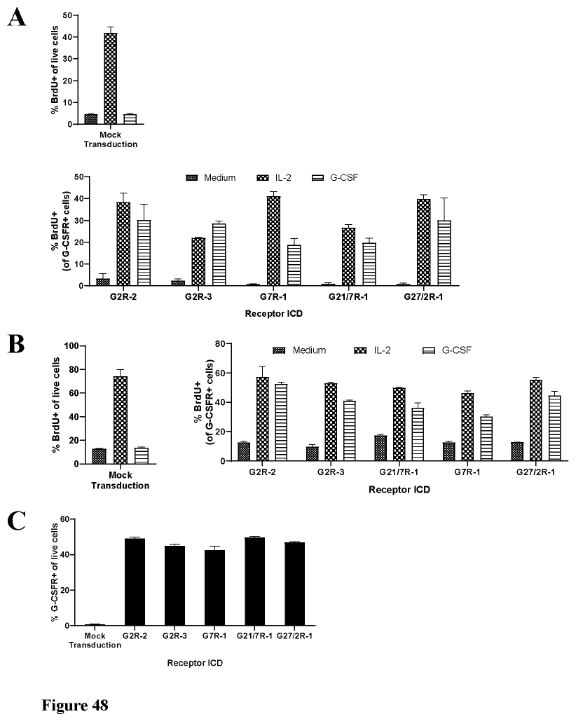

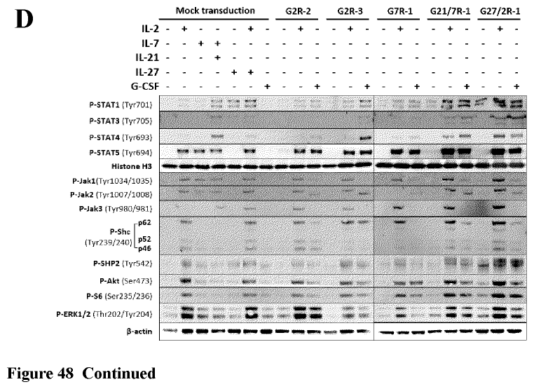

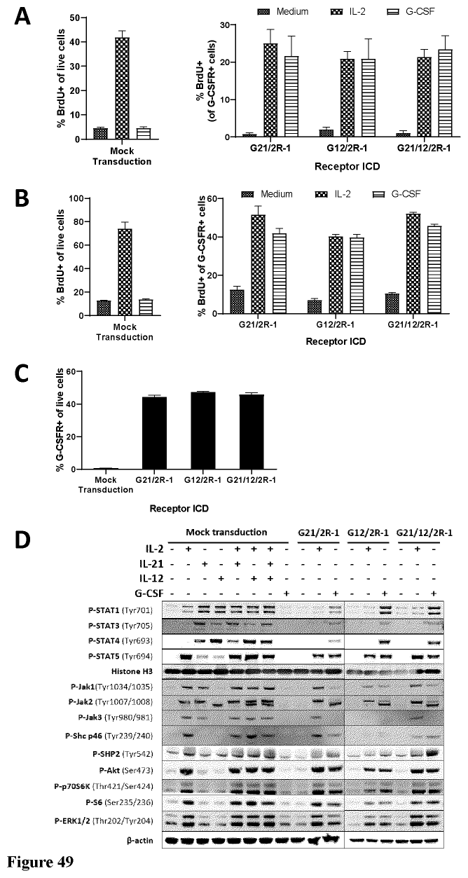

To assess cytokine signaling events and cell proliferation mediated by chimeric cytokine receptors, primary murine T cells were transduced with retroviral vectors encoding G2R-2, G2R-3, G7R-1, G21/7R-1, G27/2R-1, G21/2R-1, G12/2R-1 or G21/12/2R-1. BrdU incorporation assays were performed to assess cell cycle progression upon stimulation with G-CSF. Cells were harvested, washed, and re-plated in IL-2 (300 IU/ml), wildtype G-CSF (100 ng/ml) or no cytokine. G-CSF-induced-cell cycle progression was seen in primary murine T cells expressing G2R-2, G2R-3, G7R-1, G21/7R-1 or G27/2R-1 (FIGS. 48A, 48B) or G21/2R-1, G12/2R-1 or G21/12/2R-1 (FIGS. 49A, 49B). Expression of the G-CSFR ECD was also detectable by flow cytometry (FIGS. 48C, 49C).

By Western blot, multiple cytokine signaling events were observed in response to G-CSF (100 ng/ml) in cells expressing the indicated chimeric cytokine receptors, but not in mock transduced cells (FIGS. 48D, 49D). In general, the observed cytokine signaling events were as expected based on the signaling domains that were incorporated into the various ICD designs (FIGS. 23 and 24). As one example, the G7R-1 chimeric receptor induced phosphorylation of STAT5 (FIG. 48D), which is expected due to the incorporation of the STAT5 binding site from IL-7Rα, (FIG. 23). As a second example, the G21/2R-1 chimeric receptor induced phosphorylation of STAT3 (FIG. 49D), which is expected due to the incorporation of the STAT3 binding site from G-CSFR (FIG. 24). As a third example, the G12/2R-1 chimeric receptor induced phosphorylation of STAT4, which is expected due to the incorporation of the STAT4 binding site from IL-12Rβ2 (FIG. 24). Other chimeric cytokine receptors showed other distinct patterns of intracellular signaling events.

The results show that G2R-2, G2R-3, G7R-1, G21/7R-1, G27/2R-1, G21/2R-1, G12/2R-1 and G21/12/2R-1 are capable of inducing cytokine signaling events and proliferation in primary murine T cells upon stimulation with G-CSF. Furthermore, different patterns of intracellular signaling events can be generated by incorporating different signaling domains into the ICD of the chimeric receptor.

### Example 29: Orthogonal G-CSF Induces Expansion, Proliferation, Cytokine Related Intracellular Signaling and Immunophenotype in Primary Human T Cells Expressing G12/2R-1 with Orthogonal ECD

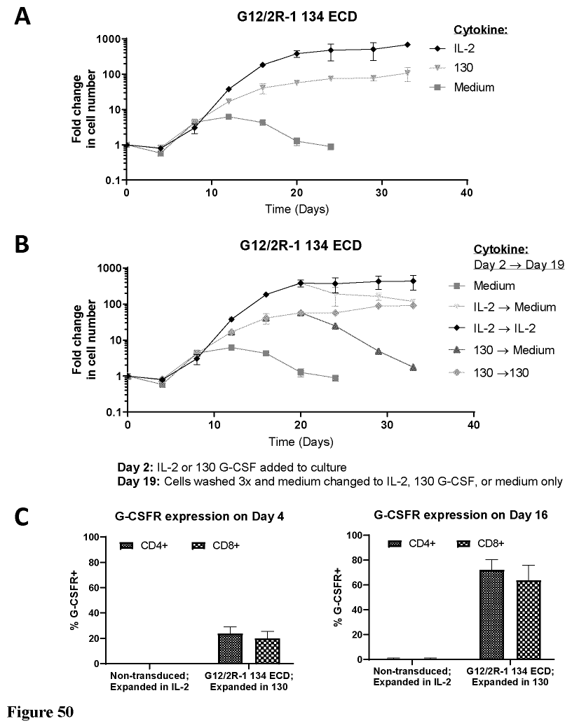

To determine if the chimeric cytokine receptor G12/2R-1 with 134 ECD was capable of inducing proliferation and expansion in response to stimulation with the orthogonal ligand 130 G-CSF, primary PBMC-derived human T cells were transduced with a lentiviral vector encoding G12/2R-1 134 ECD. T-cell growth assays were performed to assess the fold expansion of cells when cultured with IL-2 (300 IU/ml), 130 G-CSF (100 ng/ml) or no cytokine. Live cells were counted every 4-5 days. Primary human T cells expressing G12/2R-1 134 ECD expanded in culture in response to IL-2 or 130 G-CSF (FIG. 50A) but showed limited, transient expansion in medium alone.

On day 19 of this experiment, T cells that had been expanded in 130 G-CSF or IL-2 were washed three times are re-plated in IL-2, 130 G-CSF or medium alone. In medium alone, T cells showed reduced viability and a decline in number (FIG. 50B). In contrast, T cells re-plated in IL-2 or G-CSF 130 showed continued viability and stable numbers.

Expression of the G12/2R-1 134 ECD, detected by flow cytometry with an antibody against the G-CSF receptor, increased between Day 4 and Day 16 on both CD4+ and CD8+ T cells expanded by stimulation with 130 G-CSF (FIG. 50C). BrdU incorporation assays were performed to assess cell cycle progression upon stimulation with 130 G-CSF.

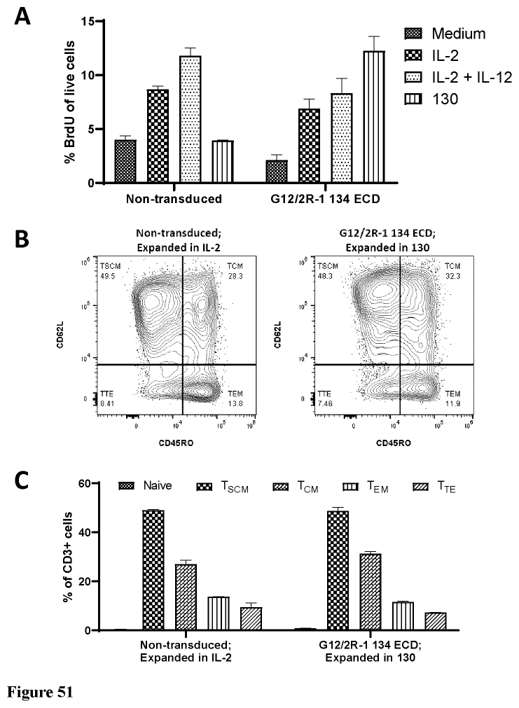

To assess cell cycle progression by BrdU assay, cells were harvested from the expansion assay, washed, and re-plated in IL-2 (300 IU/ml), IL-2 and IL-12 (10 ng/mL), 130 G-CSF (300 ng/ml) or no cytokine. Primary human T cells expressing G12/2R-1 134 ECD demonstrated cell cycle progression in response to 130 G-CSF, IL-2 or IL-2+IL-12, whereas non-transduced cells responded only to IL-2 or IL-2+IL-12 (FIG. 51A).

After a 16-day culture period, immunophenotyping was performed by flow cytometry with antibodies to CD62L and CD45RO to compare T cells expressing G12/2R-1 134 ECD expanded in 130 G-CSF to non-transduced cells expanded in IL-2. The two T cell populations showed similar proportions of stem cell-like memory (T_{SCM}), central memory (T_{CM}), effector memory (T_{EM}), and terminally differentiated (T_{TE}) phenotypes (FIG. 51B, C).

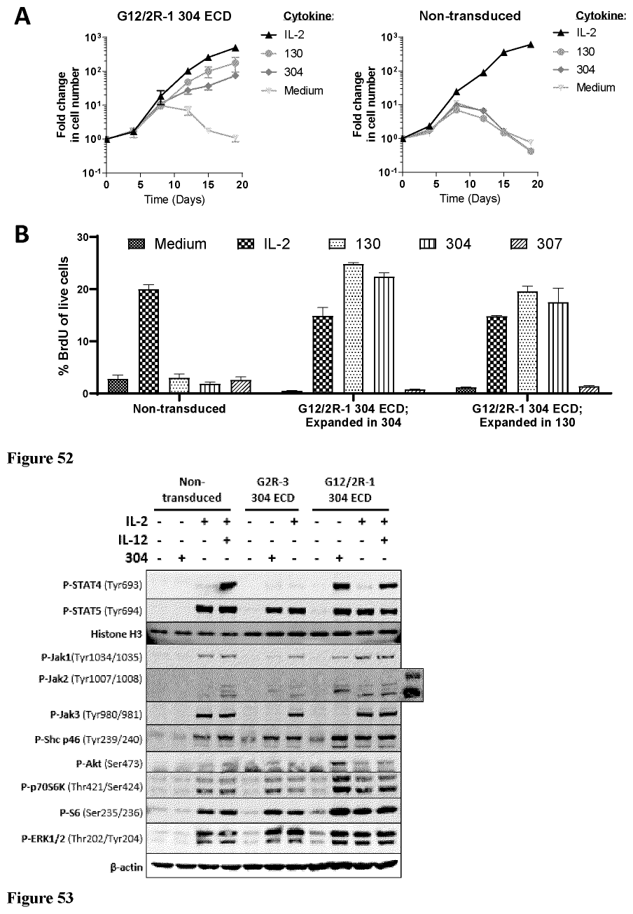

Similar experiments were performed with the chimeric cytokine receptor G12/2R-1 with 304 ECD (as opposed to 134 ECD). Primary PBMC-derived human T cells were transduced with a lentiviral vector encoding G12/2R-1 304 ECD. T-cell growth assays were performed to assess the fold expansion of cells when cultured with IL-2 (300 IU/ml), 130 G-CSF (100 ng/ml), 304 G-CSF (100 ng/mL) or medium alone. Live cells were counted every 4-5 days. T cells expressing G12/2R-1 with 304 ECD could expand in the presence of IL-2, 130 G-CSF or 304 G-CSF, but not in medium alone, while non-transduced cells could expand only in response to IL-2 (FIG. 52A).

To assess cell cycle progression by BrdU assay, T cells expressing G12/2R-1 304 ECD, previously expanded in either 130 G-CSF or 304 G-CSF, were harvested from the expansion assay, washed, and re-plated in IL-2 (300 IU/ml), 130 G-CSF (100 ng/ml), 304 G-CSF (100 ng/mL), 307 G-CSF (100 ng/mL), or medium alone. T cells expressing G12/2R-1 304 ECD demonstrated cell cycle progression in response to 130 or 304 G-CSF, but not in response to 307 G-CSF or medium alone (FIG. 52B).

The results show that G12/2R-1 134 ECD is capable of inducing cell cycle progression and expansion of primary human CD4+ and CD8+ T cells upon stimulation with orthogonal 130 G-CSF. The T cell memory phenotype of cells expressing G12/2R-1 134 ECD and expanded with 130 G-CSF is similar to non-transduced cells expanded with IL-2. In addition, G12/2R-1 304 ECD is capable of inducing selective cell cycle progression and expansion of T cells upon stimulation with 130 or 304 G-CSF, but not in response to 307 G-CSF.

### Example 30: Orthogonal G-CSF Induces Distinct Intracellular Signaling Events in Primary Human T Cells Expressing G2R-3 or G12/2R-1 with Orthogonal ECD

To assess intracellular signaling events, primary PBMC-derived human T cells were transduced with lentiviral vectors encoding G2R-3 304 ECD or G12/2R-1 304 ECD. Western blots were performed to assess intracellular cytokine signaling in cells expressing G2R-3 304 ECD or G12/2R-1 304 ECD, or non-transduced cells, upon stimulation with 304 G-CSF (100 ng/mL), IL-2 (300 IU/mL), IL-2 and IL-12 (10 ng/mL) or medium alone. In both transduced and non-transduced T cells, strong phosphorylation of STAT5 was detected in response to stimulation with either IL-2+IL-12, or IL-2 alone (FIG. 53). Strong phosphorylation of STAT4 was detected in response to stimulation with both IL-2+IL-12, but only weak phosphorylation of STAT4 was detected in response to stimulation with IL-2 alone. In cells expressing G2R-3 304 ECD, weak phosphorylation of STAT4 and strong phosphorylation of STAT5 was detected in response to stimulation with 304 G-CSF, similar to the pattern seen in response to IL-2 alone. In cells expressing G12/2R-1 304 ECD, strong phosphorylation of STAT4 and STAT5 was detected in response to stimulation with 304 G-CSF, similar to the pattern seen in response to IL-2+IL-12. Non-transduced T cells showed no response to 304 G-CSF.

The results show that G12/2R-1 with 304 ECD is capable of inducing cytokine signaling events, including strong phosphorylation of STAT4 and STAT5, in response to stimulation with 304 G-CSF. A different pattern of signaling events is seen in cells expressing G2R-3 304 ECD after stimulation with 304 G-CSF, including strong phosphorylation of STAT5 but not STAT4.

### Example 31: Design of Novel G-CSF Variants

The invention described herein is that of engineered variant G-CSF cytokines that bind selectively to a corresponding engineered extracellular domain (ECD) of G-GCSF receptor (G-CSFR). The threshold for selectivity is established using cell-based assays that measure the degree of binding and induced cell proliferation between variant G-CSF and WT G-CSFR, and between WT G-CSF and variant G-CSFR. As described herein, a panel of first and second generation engineered G-CSF/G-CSFR pairs were developed. In this example, we disclose the third generation of G-CSF designs (130a1, 130b1, 130a2, 130b2, 130a1b1) (Table 4A) which show improved thermal stability and selectivity.

Second generation G-CSF 130 variant contains three amino acid substitutions (E46R, L108K and D112R) relative to WT G-CSF. To identify additional residues that when mutated had the potential to reduce cytokine binding to WT G-CSFR, we first analyzed the biophysical and cell-based data from all our previously designed G-CSF variants, using the G-CSF/G-CSFR co-crystal structure for further guidance. Through iterative analysis, tandem glutamate residues (E122 and E123) were identified as lead candidates for substitution. Notably, these two glutamates, in addition to E46R, L108K and D112R, were substituted to arginines in the previously designed second generation G-CSF 134 variant, which proved to be unstable. Here, we individually substituted E122 and E123 using a “charge-swapping” strategy with arginine or lysine. We also substituted E122 and E123 simultaneously with lysine. Based on the structural analysis, the new positively charged residues at positions 122 and/or 123 on the cytokine are expected to repel the corresponding positively charged arginine at position 141 on the WT G-CSFR ECD leading to reduced binding and therefore enhanced selectivity. By modifying only one position (i.e., E122 or E123), we expected to reduce the stability problems observed with the previously designed 134 G-CSF in which both positions were simultaneously substituted. Furthermore, based on structural analysis, the incorporation of lysine (instead of arginine) at positions 122 and 123 was expected to yield a more stable variant than G-CSF 134. These new G-CSF variants (designated as 130a1, 130b1, 130a2, 130b2, 130a1b1) (Table 4A) were expected to efficiently pair with G-CSFR 134 ECD due to the complementary R141E substitution.

### Example 32: Recombinant Production of WT and Engineered Variant G-CSF Cytokines in E. coli

To recombinantly produce WT and engineered G-CSF cytokine variants, an E. coli-based expression system was used to generate inclusion bodies from which correctly folded proteins of interest were purified by refolding.

### Methods

Wild-type and engineered G-CSF variants were cloned into pET E. coli expression vectors under the control of the T7 viral promoter. Preparative scale E. coli production of recombinant cytokine was performed in 2 L cultures of BL21 (DE3) cells, and expression was induced via lactose-based autoinduction, controlled by natural operation of the lac operon in ZYP-5052 autoinduction media, after which cell cultures were grown for approximately 16 hrs at 30° C. with shaking at 200 rpm. Cells were then pelleted by centrifugation at 8,200 RCF for 10 minutes and the supernatant was discarded. Cell pellets (˜10 g) were resuspended in 40 ml lysis buffer (50 mM TRIS pH8, 1 mM EDTA), followed by addition of Protease inhibitor cocktail III (Sigma Aldrich, cat. 539134). E. coli cells were lysed via French press and lysates were diluted to 250 ml with lysis buffer before being centrifuged at 30,000 RCF for 30 minutes. The supernatant was discarded, and the pellet, containing the inclusion bodies, was thoroughly resuspended in 70 ml wash buffer #1 (50 mM TRIS pH8, 5 mM EDTA, 1% triton x-100) and centrifuged again. The cell pellet was then wash twice more; once with 70 ml wash buffer #2 (50 mM TRIS pH8, 5 mM EDTA, 1% Sodium deoxycholate), and once with 70 ml wash buffer #3 (50 mM TRIS pH8, 5 mM EDTA, 1M NaCl). Finally, inclusion bodies were resuspended in 100 ml solubilization buffer (50 mM TRIS pH10, 8M urea, 10 mM 2-mercaptoethanol). This solution was stirred at room temperature for 1-2 h to ensure complete solubilization of inclusion bodies and then centrifuged at 30,000 RCF for 30 minutes to remove any remaining insoluble material. The solubilized inclusion bodies were then diluted to 500 ml with dialysis buffer #1 (50 mM TRIS pH8, 1M urea, 100 m mM NaCl, 5 mM reduced glutathione). Solubilized G-CSF was dialyzed against 3 L of dialysis buffer #1 for 40 h at 4° C., using dialysis tubing with a molecular weight cutoff (mwco) of 8,000 Da (BioDesign Inc., cat. D104), followed by dialysis against 3 L of dialysis buffer #2 (50 mM TRIS pH8, 100 m mM NaCl, 1 mM reduced glutathione) for 8 h at 4° C. and finally, dialysis against 3 L of dialysis buffer #3 (25 mM Sodium acetate pH4, 50 mM NaCl) for 16 h at 4° C. Refolded G-CSF was concentrated using an Amicon® Ultra-15 centrifugal filter unit with a 10 kDa mwco (Millipore Sigma, cat. UFC901024) and further purified via cation exchange chromatography using an ENrich™ S 5×50 column (Bio-Rad, cat. 7800021) equilibrated in dialysis buffer #3 and eluted with a gradient of 50 mM to 500 mM NaCl. Fractions containing refolded G-CSF were pooled and concentrated prior to being loaded onto an ENrich™ SEC70 10×300 size-exclusion column (Bio-Rad, cat. 7801070) equilibrated in 25 mM Sodium Acetate pH 4.0, 150 mM NaCl (FIG. 1). Protein containing fractions were analyzed by reducing SDS-PAGE, pooled and the concentration was measured by nanodrop A280 measurement using an extinction coefficient of 0.836.

### Results

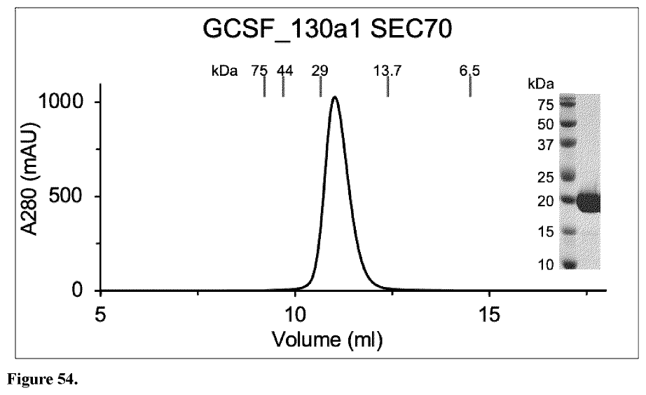

Wild-type and engineered G-CSF variants eluted at the expected volume relative to standard from the SEC 70. consistent with correctly folded protein that displayed sufficient stability to withstand chromatographic purification. Moreover, there was no observed aggregate protein peak, further confirming the effectiveness of the refolding protocol. A representative SEC UV trace and SDS-PAGE gel are shown for G-CSF_130a1 (FIG. 54). The yield per Litre of culture ranged from 2.5-5 mgs of purified, refolded protein, depending on the G-CSF variant.

### Example 33: Determination of the Thermal Stability of Engineered G-CSF Variants

Differential scanning fluorimetry (DSF) was used to measure the thermal stability of the refolded G-CSF variants compared to WT G-CSF. DSF measures protein unfolding by monitoring changes in fluorescence of a dye with affinity for hydrophobic parts of the protein, which are exposed as the protein unfolds.

### Methods

Differential scanning fluorimetry was performed using the Applied Biosystems StepOnePlus™ RT-PCR instrument (ThermoFisher Scientific, cat. 4376600) with the excitation and emission wavelengths set to 587 and 607 nm, respectively. Briefly, 20 μl of purified G-CSF (WT or variant) at a concentration of 1 mg/mL was dispensed into a 96-well plate in triplicate. To approximate the clinically validated formulation used for therapeutic WT G-CSF (10 mM Sodium acetate pH4, 5% sorbitol, 0.004% polysorbate-20) (PMID: 17822802), the assay buffer used for DSF was 10 mM Sodium acetate pH4, 25 mM NaCl. SYPRO orange (Invitrogen) was diluted to 2× concentration, from a 5,000× stock. For thermal stability measurements, the temperature scan rate was fixed at 0.5° C./min and the temperature range spanned 20° C. to 95° C. Data analysis was performed using Protein Thermal Shift Software v1.4 (ThermoFisher Scientific, cat. 4466038), which determined melting temperatures (Tms) of individual replicates by fitting fluorescence data to a two-state Boltzman model, after which triplicate Tms for each variant were then averaged.

### Results

All variant G-CSF designs tested were found to have melting temperatures 10-17° C. higher than WT G-CSF when measured in a clinically relevant buffer (Table 6A), demonstrating the significantly improved thermostability conferred by the engineered mutations.

TABLE 6A
Melting temperature (Tm) for WT and engineered
G-CSF variants determined by DSF.

| GCSF | Tm (° C.) |
|---|---|
| WT | 50.55 ± 0.34 |
| 130 | 61.96 ± 0.12 |
| 130a1 | 67.32 ± 0.02 |
| 130b1 | 66.73 ± 0.24 |
| 130a2 | 65.84 ± 0.02 |
| 130b2 | 66.38 ± 0.07 |

*determined in 10 mM sodium acetate pH 4.0, 25 mM NaCl

### Example 34: Proliferation of Human and Mouse Cell Lines Expressing Wild-Type G-CSF Receptor in Wild-Type G-CSF or Engineered Cytokines 130, 130a1, 130a2, 130b1, 130b2 and 130a1b1

Cytokines that were predicted to be less cross-reactive than 130 G-CSF for binding to the WT G-CSFR ECD, yet exhibited acceptable biophysical properties and thermal stability by in vitro assays in Examples 31 to 33, were tested for their ability to induce proliferation of the cell lines OCI-AML1 (which naturally expresses WT human G-CSFR) and 32D clone 3 (which naturally expresses WT murine G-CSFR.

### Methods

The OCI-AML1 cell line (DSMZ) was maintained in alpha-MEM medium (Gibco) containing 20% fetal bovine serum (Sigma), 20 ng/ml human GM-CSF (Peprotech) and 1% penicillin and streptomycin (Gibco). The 32D clone 3 cell line (ATCC), hereafter referred to as “32D”, was maintained in RPMI 1640 (ATCC modification) medium (Gibco) containing 10% fetal bovine serum, 1 ng/ml murine IL-3 (Peprotech) and 1% penicillin and streptomycin.

To perform the BrdU incorporation assay, OCI-AML1 or 32D cells were harvested and washed twice in PBS and once in alpha-MEM or RPMI, respectively. Cells were re-plated in fresh complete medium containing the indicated assay cytokines, at the indicated concentrations, and incubated for 48 hours at 37° C., 5% CO₂. The BrdU assay procedure followed the instruction manual for the BD Pharmingen™ FITC BrdU Flow Kit (557891), with the following addition: viability staining was performed using the eFluor™ 450 Fixable Viability Dye (eBioscience™) at a concentration of 1:600. Flow cytometry was performed using a Cytek™ Aurora instrument.

### Results

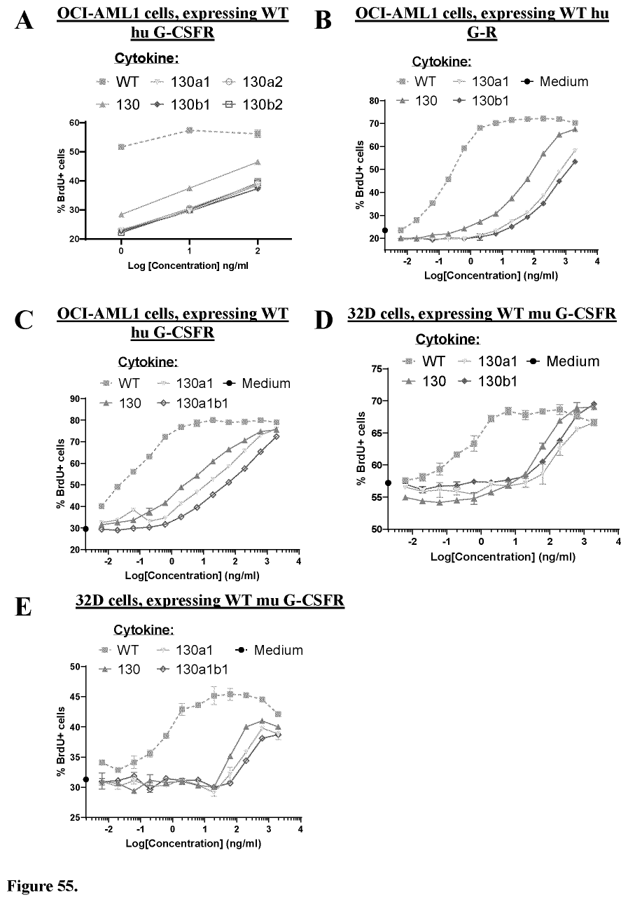

In a BrdU incorporation assay utilizing the OCI-AML1 cell line, the cells exhibited lower proliferation in response to the G-CSF variants 130, 130a1, 130a2, 130b1 and 130b2 compared to WT G-CSF (FIG. 55A). Moreover, the cells exhibited lower proliferation in response to the G-CSF variants 130a1, 130a2, 130b1 and 130b2 compared to 130 (FIG. 55A).

The cytokines WT G-CSF and G-CSF variants 130, 130a1 and 130b1 were further evaluated in a BrdU assay using a wider range of cytokine concentrations (FIG. 55B). Compared to WT G-CSF, much higher concentrations of the three variant cytokines were required to induce proliferation of OCI-AML1 cells. Compared to 130 G-CSF, higher concentrations of 130a1 and 130b1 G-CSF were required to induce proliferation of OCI-AML1 cells (FIG. 55B).

An independent BrdU assay was performed to compare WT G-CSF and G-CSF variants 130, 130a1 and 130a1b1 (FIG. 55C). Compared to WT G-CSF, much higher concentrations of the three variant cytokines were required to induce proliferation of OCI-AML1 cells. Compared to 130 G-CSF, higher concentrations of 130a1 and 130a1b1 G-CSF were required to induce proliferation of OCI-AML1 cells (FIG. 55C).

Similar results were seen in a BrdU assay involving 32D cells (FIG. 55D,E). Compared to WT G-CSF, much higher concentrations of G-CSF variants 130, 130a1, 130b1 and 130a1b1 were required to induce proliferation of 32D cells (FIG. 55D,E).

Thus, compared to human or murine WT G-CSF, G-CSF variants 130, 130a1, 130b1, 130a2, 130b2 and 130a1b1 showed significantly reduced capacity to induce proliferation in cells that express human or murine WT G-CSFR. Furthermore, compared to 130 G-CSF, the variants 130a1, 130b1, 130a2, 130b2 and 130a1b1 showed reduced capacity to induce proliferation of the OCI-AML1 cell line.

### Example 35: Proliferation of Primary Human T Cells Expressing G12/2R-1 with 134 ECD in Engineered Cytokines 130, 130a1, 130a2, 130b1, 130b2 and 130a1b1

The G-CSF variants 130, 130a1, 130a2, 130b1, 130b2 and 130a1b1 were tested for their ability to induce proliferation of PBMC-derived human T cells expressing G12/2R-1 134 ECD.

### Methods

The G12/2R-1 134 ECD cDNA was synthesized and cloned into a lentiviral transfer plasmid. The transfer plasmid and lentiviral packaging plasmids (psPAX2, pMD2.G, Addgene) were co-transfected into the lentiviral packaging cell line HEK293T/17 (ATCC) as follows. Cells were plated overnight in Opti-MEM™ I Reduced Serum Medium, GlutaMAX™ Supplement (Gibco™) containing 5% fetal bovine serum and 0.2 mM sodium pyruvate. Plasmid DNA and Lipofectamine™ 3000 Transfection Reagent (Gibco™) were mixed according to the manufacturer's specifications and added dropwise onto cells. The cells were incubated for 6 hours at 37° C., 5% CO₂, followed by replacement of the medium and further incubation overnight. The following day, the cellular supernatant was collected from the plates, stored at 4° C., and medium replaced. The following day, cellular supernatant was again collected from the cells, and pooled with supernatant from the previous day. The supernatant was centrifuged briefly to remove debris and filtered through a 0.45 μm filter. The supernatant was spun for 90 minutes at 25,000 rpm using a SW-32Ti rotor in a Beckman Optima L-XP Ultracentrifuge. The supernatant was removed, and the pellet was resuspended with 30 minutes of gentle shaking in Opti-MEM I medium.

Human T cells were isolated from a healthy donor leukapheresis product as follows. Peripheral blood mononuclear cells (PBMCs) were isolated using density gradient centrifugation. CD4+ and CD8+ T cells were isolated using human CD4 and CD8 MicroBeads (Miltenyi Biotec) following the manufacturer's specifications. Isolated T cells were plated at 5×10⁵ per well in a 48-well plate in TexMACS™ medium (Miltenyi Biotec) containing 3% human serum (Sigma) and 0.5% gentamicin (DIN 0226853) (hereafter referred to as “complete TexMACS”). Human T cell TransAct™ (Miltenyi Biotec) was added (10 μl per well), and cells were incubated at 37° C., 5% CO₂. Twenty to 28 hours following activation, T cells were transduced with lentivirus encoding the G12/2R-1 134 ECD construct. Twenty-four hours after transduction, fresh medium containing 130a1 G-CSF (100 ng/ml) was added to the T cells. Thereafter, cell cultures were replenished with fresh medium and/or cytokine every two days and kept at a density of approximately 5×10⁵ to 1×10⁶ cells per ml.

For the BrdU incorporation assay, T cells were harvested on day 10-14 of expansion and washed twice in PBS and once in complete TexMACS. Cells were re-plated in complete TexMACS containing medium alone or medium plus the following cytokines: IL-2 (300 IU/ml), IL-2+IL-12 (10 ng/ml each), or individual G-CSF variants (130, 130a1, 130b1, 130a2, 130b2 or 130a1b1, each at 100 ng/ml). T cells were incubated for 48 hours at 37° C., 5% CO2. The rest of the BrdU assay was performed as described in Example 34, except that cells were additionally stained with anti-human G-CSFR APC-conjugated and CD3 BV750-conjugated antibodies.

### Results

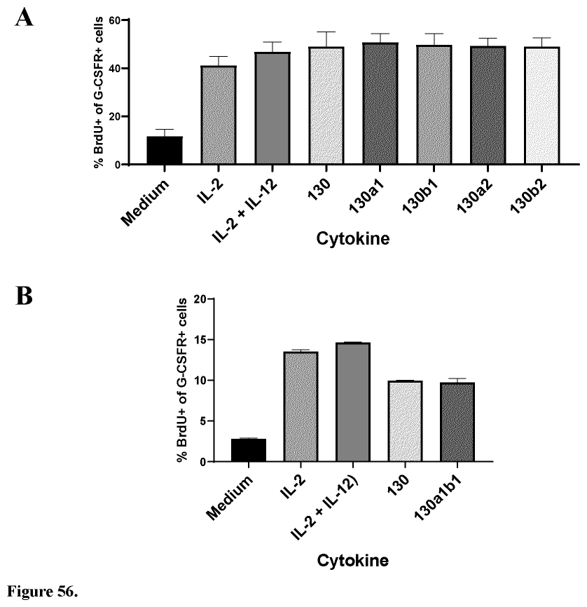

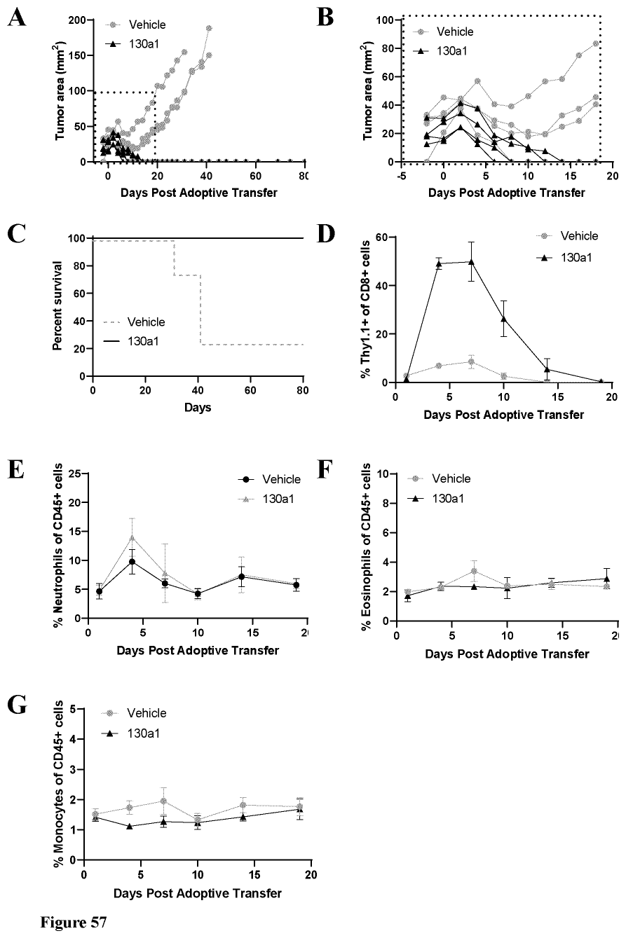

Compared to IL-2 or IL-2+IL-12, G-CSF variants 130, 130a1, 130b1, 130a2, 130b2 and 130a1b1 all induced a similar level of proliferation of human T cells expressing G12/2R-1 134 ECD (FIG. 56A). Negligible proliferation was seen with medium alone. Similar results were obtained in a repeat BrdU assay with G-CSF variants 130 and 130a1b1 (FIG. 56B).

Thus, G-CSF variants 130, 130a1, 130b1, 130a2, 130b2 and 130a1b1 can efficiently induce proliferation of T cells expressing the chimeric receptor G12/2R-1 134 ECD, resulting in a proliferative response similar to that elicited by IL-2 or IL-2+IL-12.

### Example 36: In a Murine Mammary Carcinoma Model, Treatment with Variant Cytokine 130a1 Selectively Enhances the Expansion and Anti-Tumor Activity of Tumor-Specific T Cells Expressing G2R-3 134 ECD

Based on the ability of 130a1 G-CSF to stimulate proliferation of T cells expressing G12/2R-1 134 ECD, and its low cross-reactivity with WT murine and human G-CSFR, it was advanced to in vivo testing in a murine breast cancer model.

### Methods

All procedures followed the Canadian Council for Animal Care guidelines and were approved by the University of Victoria Animal Care Committee. C57Bl/6 mice expressing the Neu^{OT-IOT-II} transgene in mammary epithelium under the control of the MMTV promoter were utilized as tumor hosts (Wall et al., 2007). Tumor-specific CD8+ T cells were obtained from T Cell Receptor (TCR) transgenic OT-I mice (hereafter referred to as “OT-I” mice; Jackson Laboratories); OT-I T cells recognize residues 257-264 of chicken ovalbumin presented by MHC class I. We also obtained congenic mice harboring the T lymphocyte-specific Thy1a (Thy1.1) allele (hereafter referred to as “Thy1.1+” mice; Jackson Laboratories). The OT-I and Thy1.1+ strains were intercrossed to homozygosity to produce “Thy1.1+ OT-I” mice; this allows the Thy1.1 marker to be used to identify OT-I T cells by flow cytometry. NOP23 is a syngeneic mammary tumor line that was derived from a spontaneous tumor in a transgenic mouse expressing Neu^{OT-I/OT-II} and a dominant negative version of p53 in mammary epithelium (Wall et al., 2007) (Yang et al., 2009). NOP23 cells present residues 257-264 of chicken ovalbumin on MHC class I and hence can be recognized by OT-I T cells. NOP23 cells were maintained at an early passage and were cultured in DMEM-high glucose (Hyclone) containing 10% fetal bovine serum, 1× Insulin-Transferrin-Selenium supplement (Corning), and 1% penicillin/streptomycin (Gibco). The cell line underwent routine testing for mouse pathogens and Mycoplasma species.

Host MMTV Neu^{OT-I/OT-II} mice were implanted subcutaneously with 1×10⁶ NOP23 cells on the rear flank. Once tumors had reached a mean area of 20-50 mm² (approximately 15 days following implantation), mice received an intravenous infusion of 5×10⁶ donor Thy1.1+OT-I lymphocytes that had been retrovirally transduced to express G2R-3 134 ECD as described in the following paragraphs.

The retroviral packaging cell line Platinum-E (Cell Biolabs, RV-101) was cultured in DMEM containing 10% FBS, 1% penicillin/streptomycin, puromycin (1 μg/ml), and blasticidin (10 μg/ml). Chimeric receptor constructs were synthesized and cloned into a pMIG retroviral transfer plasmid (plasmid #9044, Addgene) that had been altered by restriction endonuclease cloning to remove the IRES-GFP (BglII to PacI sites) and to introduce annealed primers encoding a custom multiple cloning site. The resulting sequences were verified by Sanger sequencing. The transfer plasmid was transfected into Platinum-E cells using the Lipofectamine 3000 transfection method, as described above. The retroviral supernatant was collected from the plates at 24 and 48 hours following transfection and filtered through a 0.45 micron filter. Hexadimethrine bromide (1.6 μg/ml, Sigma-Aldrich) and human IL-2 (300 IU/ml) were added to the supernatant. The purified retroviral supernatant was used to transduce murine lymphocytes as described below.

Forty-eight hours prior to collection of retroviral supernatant, 24-well adherent plates were coated with unconjugated anti-murine CD3 (10 μg/ml, BD Biosciences, 553058) and anti-murine CD28 (2 μg/ml, BD Biosciences, 553294) antibodies, diluted in PBS, and stored at 4° C. Twenty-four hours prior to collection of retroviral supernatant, Thy1.1+ OT-I mice were euthanized, and T cells were harvested from spleens as follows. Spleens were manually dissociated and filtered through a 100 micron filter. Red blood cells were lysed by incubation in ACK lysis buffer (Gibco, A1049201) for five minutes at room temperature, followed by one wash in serum-containing medium. CD8a-positive T cells were isolated using a bead-based isolation kit (Miltenyi Biotec, 130-104-075). Cells were added to plates coated with anti-CD3- and anti-CD28 antibodies in murine T cell expansion medium (RPMI-1640 containing 10% FBS, penicillin/streptomycin, 0.05 mM β-mercaptoethanol, and 300 IU/mL human IL-2), and incubated at 37° C., 5% CO₂ for 24 hours.

Retroviral transduction was performed on two successive days (Day 1 and 2) as follows. Approximately half of the medium was replaced with retroviral supernatant (generated as described above). Cells were spinfected with the retroviral supernatant at 1000 g, for 90 minutes, at 30° C. Plates were returned to the incubator for 0-4 hours, and then approximately half of the medium was replaced with fresh T cell expansion medium. The procedure was repeated the next day. Twenty-four hours after the second transduction, T cells were split into 6-well plates, removed from antibody stimulation, and cultured under standard conditions.

On Day 5, the transduced T cells were harvested, and the transduction efficiency was assessed by flow cytometry with antibodies to human G-CSFR and CD8a (PerCP-eFluor 710 conjugate, eBioscience™, 46-0081-82), as well as Fixable Viability Dye eFluor™ 506 (1:1000 dilution), as described above. Cells were washed three times in PBS, and then infused by tail vein injection into host mice bearing established NOP23 tumors.

Following T cell infusion, mice were monitored for tumor growth and engraftment of Thy1.1+ OT-I cells in peripheral blood. Mice were randomized to receive intraperitoneal injections of vehicle versus 130a1 G-CSF (10 μg/dose) daily for 14 days, followed by every other day for a further 14 days for a total of 21 doses. Throughout the cytokine treatment period, operators were blinded to the contents of the cytokine syringes (i.e., vehicle versus 130a1 G-CSF). On Days 1, 4, 7, 10, 14 and 19 following ACT, a sample of peripheral blood was collected from each mouse. PBMCs were isolated after ACK lysis (described above), incubated in mouse Fc block and Zombie NIR viability dye (1:4,000), and stained with antibodies specific for murine CD45 (PerCP-conjugated antibody, 1:200), CD3 (AlexaFluor™ 700-conjugated antibody, 1:100), CD8a (APC-conjugated antibody, 1:80), NK1.1 (PECy7-conjugated antibody, 1:25), CD19 (SuperBright™ 780-conjugated antibody, 1:100), CD11b (FITC-conjugated antibody, 1:25), CD11c (BV510-conjugated antibody, 1:50), Ly6C (BV605-conjugated antibody, 1:10), and Ly6G (PE-conjugated antibody, 1:80). Flow cytometry was performed on a Cytek Aurora instrument.

Mice were monitored for tumor size, body weight and general health and euthanized when they reached the experimental endpoint (>150 mm² tumor area) or, for mice experiencing complete tumor regression, on Day 80 following T cell infusion.

### Results

All mice received an equivalent dose (5×10⁶) of Thy1.1+ OT-I cells transduced to express G2R-3 134 ECD. The transduction efficiency was 36%, as assessed by the percentage of Thy1.1+ cells expressing human G-CSFR by flow cytometry. In the 130a1 G-CSF-treated group, 4/4 mice experienced complete regression (CR) of their tumor and remained tumor-free until day 80, when the study was stopped for practical reasons (FIGS. 57A and 57B). In contrast, in the vehicle-treated group, only 1/4 mice experienced CR, while the remaining 3/4 mice had tumors that progressed to the experimental endpoint. This translated to survival of 100% of mice in the 130a1 G-CSF-treated group, and 25% in the vehicle-treated group (FIG. 57C).

The expansion and persistence of Thy1.1+ OT-I cells was monitored in serial blood samples collected from mice. In the 130a1 G-CSF-treated group, Thy1.1+ OT-I cells reached a mean peak engraftment of 49.8% of all CD8⁺ T cells on Day 7, whereas in the vehicle-treated group Thy1.1+ OT-I cells reached a mean peak engraftment of only 8.6% of all CD8⁺ T cells on Day 7 (FIG. 57D).

Peripheral blood samples were also assessed for the percentage of myeloid cell subsets relative to all CD45+ cells. The 130a1 G-CSF- and vehicle-treated groups showed no significant differences in the percentage of neutrophils (CD3−, CD19−, NK1.1−, CD11b+, CD11c−, Ly6G+), eosinophils (CD3−, CD19−, NK1.1−, CD11b+, CD11c−, Ly6G−, SSC^{high}) or monocytes (CD3−, CD19−, NK1.1−, CD11b+, CD11c−, Ly6G−, SSC^{low}) relative to all CD45+ cells in peripheral blood over time (FIGS. 57E, 57F and 57G).

Throughout the study, all mice in both groups maintained a normal body weight and exhibited good general health, despite tumor progression in some.

Thus, 130a1 G-CSF was able to induce selective and profound expansion of tumor-specific CD8+ T cells expressing G2R-3 134 ECD. This resulted in a 100% durable CR rate compared to 25% in the vehicle-treated group. Treatment with 130a1 G-CSF did not affect neutrophil, eosinophil or monocyte counts in peripheral blood. There were no overt toxicities associated with 130a1 G-CSF treatment.

### Example 37: In a Murine Mammary Carcinoma Model, Treatment with Variant G-CSF 130a1 Selectively Enhances the Expansion and Anti-Tumor Activity of Tumor-Specific T Cells Expressing G12/2R-1 134 ECD

The variant cytokine 130a1 G-CSF was further tested for the ability to enhance the expansion and anti-tumor activity of T cells engineered to express G12/2R-1 134 ECD in an in vivo murine breast cancer model.

### Methods

We used the same methods as described in Example 36, with the exception that Thy1.1+ OT-I cells were retrovirally transduced to express G12/2R-1 134 ECD instead of G2R-3 134 ECD.

### Results

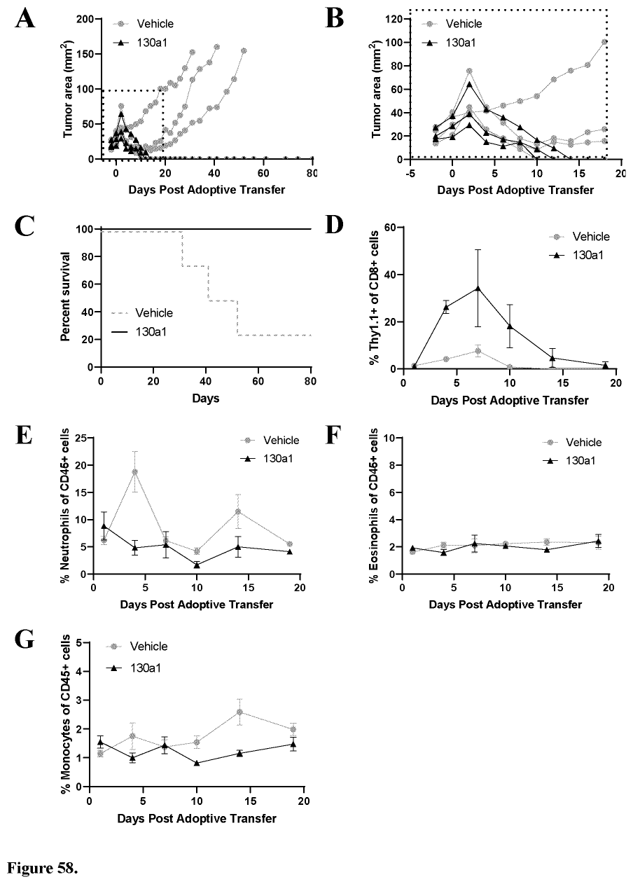

All mice received an equivalent dose (5×10⁶) of Thy1.1+ OT-I cells transduced to express G12/2R-1 134 ECD. The transduction efficiency was 43%, as assessed by the percentage of Thy1.1+ cells expressing human G-CSFR+ by flow cytometry. In the 130a1 G-CSF-treated group, 3/3 mice experienced complete regression (CR) of their tumor and remained tumor-free until day 80, when the study was stopped for practical reasons (FIGS. 58A and 58B). In contrast, in the vehicle-treated group, only 1/4 mice experienced CR, while the remaining 3/4 mice had tumors that progressed to the experimental endpoint. This translated to survival of 100% of mice in the 130a1 G-CSF-treated group, and 25% in the vehicle-treated group (FIG. 58C).

The expansion and persistence of Thy1.1+ OT-I cells was monitored in serial blood samples collected from mice. In the 130a1 G-CSF-treated group, Thy1.1+ OT-I cells reached a mean peak engraftment of 34.3% of all CD8⁺ T cells on Day 7, whereas in the vehicle-treated group Thy1.1+ OT-I cells reached a mean peak engraftment of only 7.7% of all CD8⁺ T cells on Day 7 (FIG. 58D).

Peripheral blood samples were also assessed for the percentage of several myeloid cell subsets relative to all CD45+ cells. The 130a1 G-CSF- and vehicle-treated groups showed no significant differences in the percentage of neutrophils (CD3−, CD19−, NK1.1−, CD11b+, CD11c−, Ly6G+), eosinophils (CD3−, CD19−, NK1.1−, CD11b+, CD11c−, Ly6G−, SSC^{high}) or monocytes (CD3−, CD19−, NK1.1−, CD11b+, CD11c−, Ly6G−, SSC^{low}) relative to all CD45+ cells in peripheral blood over time (FIGS. 58E, 58F and 58G).

Throughout the study, all mice in both groups maintained a normal body weight and exhibited good general health, despite tumor progression in some.

Thus, 130a1 G-CSF was able to induce selective and profound expansion of tumor-specific CD8+ T cells expressing G12/2R-1 134 ECD. This resulted in a 100% durable CR rate compared to 25% in the vehicle-treated group. Treatment with 130a1 G-CSF did not affect neutrophil, eosinophil or monocyte counts in peripheral blood. There were no overt toxicities associated with 130a1 G-CSF treatment.

### Example 38: The Therapeutic Effects of 130a1 G-CSF Require the Expression of Cognate Engineered Cytokine Receptors by Tumor-Specific OT-I T Cells

To demonstrate that 130a1 G-CSF mediates its immunological and anti-tumor effects through receptors containing the G-CSFR 134 ECD, the above in vivo experiment was performed using mock-transduced OT-I T cells (i.e., OT-I T cells that underwent the retroviral transduction procedure but without including a retroviral construct). The activity of 130a1 G-CSF was also compared to that of human IL-2.

### Methods

We used the same methods as described in Example 36, with two exceptions: (a) the Thy1.1+ OT-I cells were mock-transduced using supernatant derived from mock-transfected Platinum-Eco cells; all other aspects of the retroviral transduction process were the same; and (b) we included an additional control group of mice that received human interleukin-2 (30,000 IU/dose; Proleukin) in place of vehicle or 130a1 G-CSF, following the same treatment schedule.

### Results

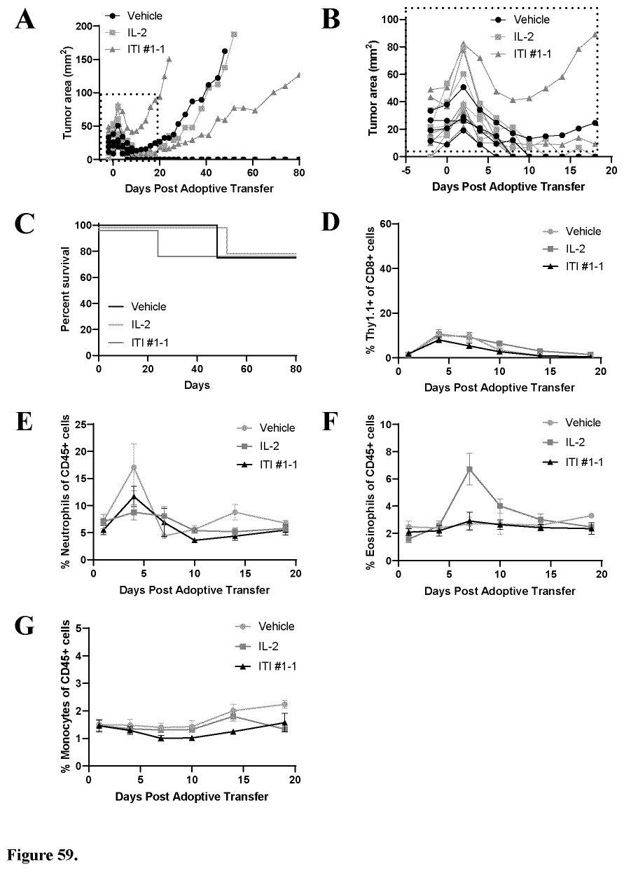

All mice received an equivalent dose (5×10⁶) of Thy1.1+ OT-I cells which had undergone the mock transduction process. Mice were then randomized to receive vehicle, 130a1 G-CSF or IL-2. In the 130a1 G-CSF-treated group, 3/5 mice experienced complete regression (CR) of their tumor and remained tumor-free until day 80, when the study was stopped for practical reasons (FIGS. 59A and 59B). In the IL-2-treated group, 4/5 mice experienced CR and remained tumor-free until day 80. In the vehicle-treated group, 3/4 mice experienced CR and remained tumor-free until day 80 (FIGS. 59A and 59B). This translated to survival of 80% of mice in the 130a1 G-CSF- and IL-2-treated groups, and 75% in the vehicle-treated group (FIG. 59C).

The complete response rates achieved by mock-transduced OT-I T cells were higher than those seen with vehicle-treated OT-I T cells expressing either G2R-3 134 ECD or G12/2R-1 134 ECD (Examples 36 and 37), despite the fact that none of these groups had the benefit of signaling through G2R-3 134 ECD or G12/2R-1 134 ECD. The lower efficacy of transduced vehicle-treated OT-I T cells likely reflects a modest impairment of T cell function caused by retroviral transduction.

The expansion and persistence of Thy1.1+ OT-I cells was monitored in serial blood samples collected from mice. Thy1.1+ OT-I cell numbers peaked on Day 4 in all three groups, reaching levels of 8.0%, 10.6% and 9.9% of all CD8⁺ T cells for the 130a1 G-CSF-, IL-2-, and vehicle-treated groups (FIG. 59D).

There were no significant differences in the number of neutrophils or monocytes as a percentage of all peripheral blood CD45+ cells between the 130a1 G-CSF-, IL-2-, or vehicle-treated groups (FIGS. 59E and 59G).

On Day 7, eosinophils reached a mean of 6.7% of all CD45+ cells in the IL-2-treated group compared to 2.9% and 2.7% in the 130a1 G-CSF- and vehicle-treated groups, respectively (FIG. 59F). Eosinophilial is a known effect of systemic IL-2 administration in mice and humans.

Throughout the study, all mice in both groups maintained a normal body weight and exhibited good general health, despite tumor progression in some.

Thus, in mice receiving mock-transduced Thy1.1+ OT-I cells, 130a1 G-CSF failed to mediate anti-tumor activity or induce T cell expansion. As described above, 130a1 G-CSF had no effect on neutrophil, eosinophil or monocyte counts in peripheral blood. There were no overt toxicities associated with 130a1 G-CSF treatment. IL-2 (at the dose and schedule used) had very modest effects on T cell expansion and anti-tumor activity despite inducing transient eosinophilia.

### Example 39: Construction and Functional Evaluation of a Chimeric Receptor with a Variant Extracellular Domain from the G-CSFR Receptor and Intracellular Signaling Domains from IL-4 Receptor Alpha

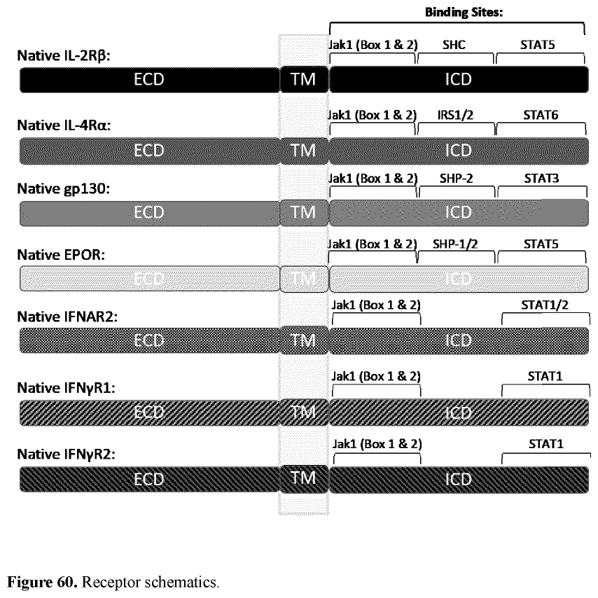

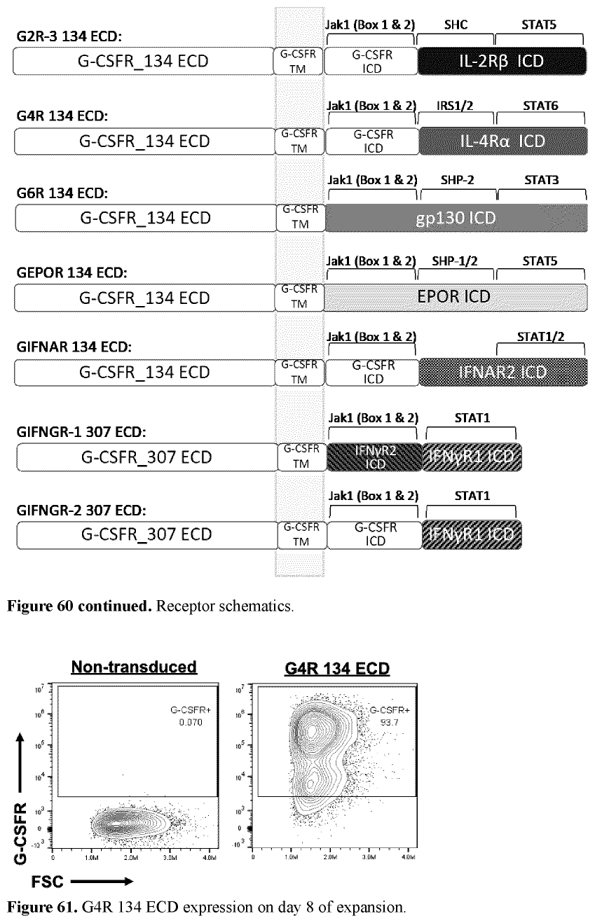

We synthesized a chimeric receptor called G4R 134 ECD (FIG. 60) which contained the extracellular domain (134 variant), transmembrane domain, and Box 1 and 2 regions of human G-CSFR fused to a portion of the intracellular domain of human IL-4 receptor alpha. The chimeric receptor subunit was encoded in a lentiviral vector which was used to transduce human CD4+ and CD8⁺ T cells. Transduced T cells (and non-transduced control T cells) were analyzed by flow cytometry to detect cell surface expression of G4R 134 ECD. The ability of G4R 134 ECD to induce T cell expansion and proliferation was evaluated in vitro. Biochemical signaling events mediated by G4R 134 ECD were evaluated by western blot using phospho-specific antibodies against STAT6 and other signaling intermediaries associated with IL-4 signaling.

### Methods

The chimeric receptor construct G4R 134 ECD was synthesized and cloned into a lentiviral transfer plasmid (Twist Bioscience). The transfer plasmid and lentiviral packaging plasmids (psPAX2, pMD2.G, Addgene) were co-transfected into the lentiviral packaging cell line HEK293T/17 (ATCC) as follows. Cells were plated overnight in Opti-MEM™ I Reduced Serum Medium, GlutaMAX™ Supplement (Gibco™) containing 5% fetal bovine serum and 0.2 mM sodium pyruvate. Plasmid DNA and Lipofectamine™ 3000 Transfection Reagent (Gibco™) were mixed, according to the manufacturer's specifications, and added dropwise onto cells. The cells were incubated for 6 hours at 37° C., 5% CO₂, followed by replacement of the medium and further incubation overnight. The following day, the cellular supernatant was collected from the plates and stored at 4° C., and the medium was replaced. The next day, cellular supernatant was again collected from the cells and pooled with supernatant from the previous day. The supernatant was centrifuged briefly to remove debris and filtered through a 0.45 μm filter. The supernatant was spun for 90 minutes at 25,000 rpm using a SW-32Ti rotor in a Beckman Optima L-XP Ultracentrifuge. The supernatant was removed, and the pellet was resuspended with 30 minutes of gentle shaking in Opti-MEM I medium.

Primary human T cells were isolated from healthy donor leukapheresis product as follows. Peripheral blood mononuclear cells (PBMCs) were isolated using density gradient centrifugation. CD4+ and CD8+ T cells were isolated using human CD4 and CD8 MicroBeads (Miltenyi Biotec) following the manufacturer's specifications. For some experiments, T cells were viably frozen and thawed later for analysis. Isolated T cells were plated at 5×10⁵ per well in a 48-well plate in TexMACS™ medium (Miltenyi Biotec) containing 3% human serum (Sigma) and 0.5% gentamicin (DIN 0226853) (hereafter referred to as “complete TexMACS”). Human T cell TransAct™ (Miltenyi Biotec) was added (10 ml per well), and cells were incubated at 37° C., 5% CO₂. Twenty to 28 hours following activation, the T cells were transduced with lentivirus encoding the receptor construct. The following day, fresh medium was added to T cells, along with either IL-2 (300 IU/ml), 130a1 G-CSF (100 ng/ml), IL-2 plus 130a1 G-CSF (300 IU/ml or 100 ng/ml, respectively) or no added cytokine. (For the non-transduced T cells, the 307 G-CSF variant was added with 130a1 G-CSF to serve as a control for another arm of the experiment [not shown]; use of this experimental convenience was justified by the fact that neither 130a1 G-CSF nor 307 G-CSF induces proliferation or other signaling events in non-transduced T cells.) T cells were maintained by addition of fresh medium and/or the indicated cytokines every two days, while maintaining a density of approximately 5×10⁵ to 1×10⁶ cells per ml.

Eight days after transduction, expression of G4R 134 ECD was assessed by flow cytometry of T cells harvested from the IL-2 culture condition. T cells were incubated for 15 minutes at 4° C. with anti-human G-CSFR APC-conjugated antibody (1:50 dilution), anti-human CD3 BV750™-conjugated antibody (1:50 dilution), anti-human CD4 PE-conjugated antibody (1:25 dilution), anti-human CD8 PerCP-conjugated antibody (1:100) and eBioscience™ Fixable Viability Dye eFluor™ 506 (1:1000 dilution). Cells were then washed twice and analyzed on a Cytek™ Aurora flow cytometer.

To assess T cell expansion, cells from each of the culturing conditions were counted on days 4, 8, 12 and 16 using a Cytek™ Aurora flow cytometer.

To assess cell cycle progression (BrdU incorporation), cells were harvested on day 12 (from the 130a1 G-CSF+IL-2 culture condition), washed twice in PBS and once in complete TexMACS medium, and re-plated in fresh complete TexMACS medium containing the indicated cytokines: IL-2 (300 IU/ml), IL-4 (50 ng/ml), IL-2+IL-4 (300 IU/ml and 50 ng/ml, respectively), 130a1 G-CSF (100 ng/ml), 130a1 G-CSF+IL-2, or medium alone (no added cytokine). Cells were cultured for 48 hours at 37° C., 5% CO₂. The BrdU assay procedure followed the instruction manual for the BD Pharmingen™ FITC BrdU Flow Kit (557891), with one exception: cells were also stained to detect cell surface expression of the G-CSFR ECD (as described in Example 35). Flow cytometry was performed using a Cytek™ Aurora instrument.

For western blot experiments, cells were harvested from the 130a1 G-CSF+IL-2 culture condition, washed three times, and rested overnight in complete TexMACS. The following day, cells were stimulated for 20 minutes at 37° C. with medium alone or medium plus IL-2 (300 IU/ml), IL-4 (50 ng/ml), or 130a1 G-CSF (100 ng/ml). Cells were washed once in a buffer containing 10 mM HEPES, pH 7.9, 1 mM MgCl₂, 0.05 mM EGTA, 0.5 mM EDTA, pH 8.0, 1 mM DTT, and 1× Pierce Protease and Phosphatase Inhibitor Mini Tablets (A32961). Cells were lysed for 10 minutes on ice in wash buffer containing 0.2% NP-40 substitute (Sigma). Lysates were centrifuged for 10 minutes at 13,000 rpm at 4° C., after which supernatant (cytoplasmic fraction) was collected. To extract the nuclear fraction, pellets from the lysis step were resuspended in the above wash buffer with the addition of 0.42M NaCl and 20% glycerol. The nuclear fraction was incubated for 30 minutes on ice, with frequent vortexing, and centrifuged for 20 minutes at 13,000 rpm at 4° C., after which the nuclear fraction (supernatant) was collected. The cytoplasmic and nuclear fractions were heated (70° C.) under reducing conditions for 10 minutes and run on NuPAGE™ 4-12% Bis-Tris Protein Gels. The gel contents were transferred to nitrocellulose membranes (60 min at 20V in a Trans-Blot® SD Semi-Dry Transfer Cell), dried, and blocked for 1 hr in Intercept® Blocking Buffer in TBS (927-600001, LI-COR). The blots were incubated with primary antibodies (1:1,000) overnight at 4° C. in Intercept® Blocking Buffer in TBS containing 0.1% Tween20. Primary antibodies were obtained from Cell Signaling Technologies: Phospho-Shc (Tyr239/240) antibody #2434, Phospho-Akt (Ser473) (D9E) XP® rabbit monoclonal antibody #4060, Phospho-S6 Ribosomal Protein (Ser235/236) antibody #2211, Phospho-p44/42 MAPK (Erk1/2) (Thr202/Tyr204) antibody #9101, β-Actin (13E5) rabbit monoclonal antibody #4970, Phospho-JAK1 (Tyr1034/1035) (D7N4Z) rabbit monoclonal antibody #74129, Phospho-JAK3 (Tyr980/981) (D44E3) rabbit monoclonal antibody #5031, Phospho-Stat6 (Tyr641) (C11C5) rabbit monoclonal antibody #9361, and Histone H3 (96C10) mouse monoclonal antibody #3638. Blots were washed three times in TBS containing 0.1% Tween20 and incubated with secondary antibodies (1:10,000) in TBS buffer containing 0.1% Tween20 for 30-60 minutes at room temperature. Secondary antibodies were obtained from Cell Signaling Technologies: anti-mouse IgG (H+L) (DyLight™ 800 4× PEG Conjugate) #5257 and anti-rabbit IgG (H+L) (DyLight™ 800 4× PEG Conjugate) #5151. Blots were washed and exposed on a LI-COR Odyssey imager.

### Results

By flow cytometry (FIG. 61), a large proportion (93.7%) of CD3+ T cells transduced with the lentiviral vector encoding G4R 134 ECD expressed the G-CSFR ECD, whereas non-transduced T cells did not.

Thus, G4R 134 ECD can be expressed on the surface of engineered T cells.

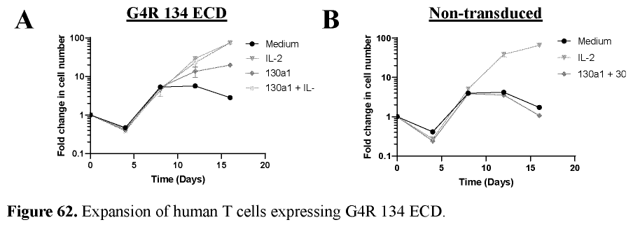

As shown in FIG. 62, T cells transduced with the lentiviral vector encoding G4R 134 ECD expanded well in medium containing IL-2 or IL-2+130a1 G-CSF and showed an intermediate level of expansion in response to 130a1 G-CSF alone (FIG. 62A). Non-transduced T cells expanded well in response to IL-2 but showed no response to the combination of 130a1 G-CSF plus the G-CSF variant 307 (the latter was included as a control for a separate arm of this experiment; not shown) (FIG. 62B). Neither culture expanded well in medium alone.

Thus, expression of G4R 134 ECD on T cells leads to increased expansion in response to 130a1 G-CSF, demonstrating that the hybrid intracellular domain of G4R 134 ECD transduces a functional growth signal in T cells.

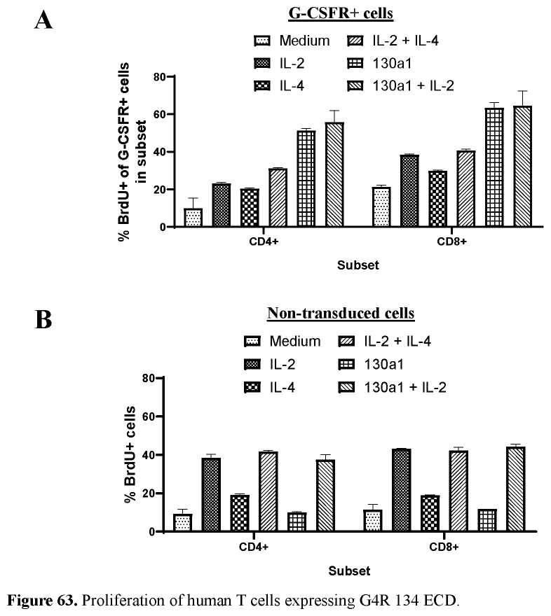

As shown in FIG. 63, CD4+ and CD8+ T cells expressing G4R 134 ECD demonstrated proliferation (BrdU incorporation) in response to IL-2, IL-4, IL-2+IL-4, 130a1 G-CSF, or 130a1 G-CSF+IL-2 (FIG. 63A). In contrast, non-transduced CD4+ and CD8+ T cells proliferated in response to IL-2, IL-4, IL-2+IL-4, and 130a1 G-CSF+IL-2 but not in response to 130a1 G-CSF alone (FIG. 63B). None of the cultures proliferated in response to medium alone, with the exception of CD8+ T cells expressing G4R 134 ECD, which had elevated background proliferation in this particular experiment.

Thus, expression of G4R 134 ECD on CD4+ and CD8+ T cells leads to increased proliferation in response to 130a1 G-CSF, achieving levels equivalent to or even exceeding that induced by IL-2 or IL-4. This demonstrates that the hybrid intracellular domain of G4R 134 ECD transduces a functional proliferative signal in T cells.

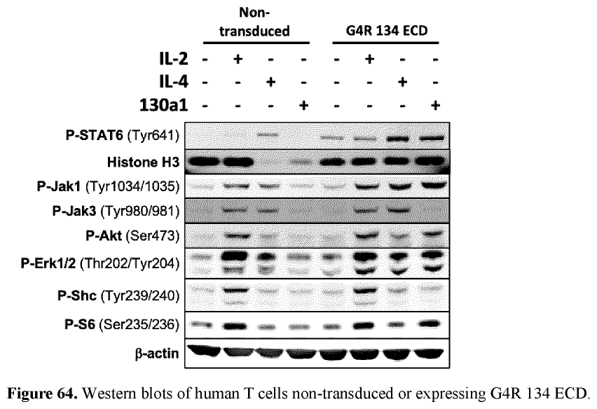

As shown in FIG. 64, in both non-transduced T cells and T cells expressing G4R 134 ECD, IL-2 induced the phosphorylation of JAK1, JAK3, Shc, Erk1/2, Akt, and S6. IL-4 induced the phosphorylation of STAT6, JAK1 and JAK3, with only modest phosphorylation of Erk1/2, Akt, and S6 and no phosphorylation of Shc. In T cells expressing G4R 134 ECD, 130a1 G-CSF induced the phosphorylation of STAT6 and JAK1, with intermediate phosphorylation of Erk1/2, Akt, and S6 (relative to IL-2), and no phosphorylation of JAK3 or Shc. The lack of phosphorylation of JAK3 is expected given that G4R 134 ECD uses the Box 1 and 2 regions of human G-CSFR, which binds JAK2 instead of JAK3. As expected, 130a did not induce any signaling events in non-transduced T cells.

Thus, the hybrid intracellular domain of G4R 134 ECD activates many of the same biochemical signaling events as the native IL-4 receptor, in particular tyrosine phosphorylation of STAT6.

### Example 40: Construction and Functional Evaluation of a Chimeric Receptor with a Variant Extracellular Domain from the G-CSFR Receptor and Intracellular Signaling Domains from IL-6 Receptor Beta (gp130)

We synthesized a chimeric receptor called G6R 134 ECD (FIG. 60) which contained the extracellular domain (134 variant), transmembrane domain, and Box 1 and 2 regions of human G-CSFR fused to a portion of the intracellular domain of human IL-6 receptor beta (gp130). The chimeric receptor subunit was encoded in a lentiviral vector which was used to transduce human CD4+ and CD8+ T cells. Transduced T cells (and non-transduced control T cells) were analyzed by flow cytometry to detect cell surface expression of G6R 134 ECD. The ability of G6R 134 ECD to induce T cell expansion and proliferation was evaluated in vitro. Biochemical signaling events mediated by G6R 134 ECD were evaluated by western blot using phospho-specific antibodies against STAT3 and STAT5.

### Methods

In general, we followed similar methods as described in Example 39; however, the experiments were adapted to focus on IL-6 signaling mechanisms. For example, IL-6 was used as a reference cytokine instead of IL-4.

Human T cells were engineered to express G6R 134 ECD and transduction efficiency was determined by flow cytometry on Day 8, as per Example 39.

T cells were expanded in IL-2 (300 IU/ml), 130a1 G-CSF (100 ng/ml), IL-2+130a1 G-CSF (300 IU/ml and 100 ng/ml, respectively) or medium alone (no added cytokine) for 16 days, with medium and the indicated cytokines refreshed every two days. On days 4, 8, 12 and 16 of the expansion, T cells were counted using a Cytek™ Aurora flow cytometer.

For the BrdU incorporation assay, on day 12 of expansion T cells were harvested from the IL-2 culture condition and washed twice in PBS and once in complete TexMACS medium. Cells were re-plated in fresh complete TexMACS medium containing the relevant assay cytokine: IL-2 (300 IU/ml), IL-6 (100 ng/ml), IL-2+IL-6 (300 IU/ml and 100 ng/ml, respectively), 130a1 G-CSF (100 ng/ml), 130a1 G-CSF+IL-2 (300 IU/ml and 100 ng/ml, respectively), or medium alone (no added cytokine). Cells were cultured for 48 hours at 37° C., 5% CO₂. The BrdU incorporation assay was performed as described in Example 39.

For the western blot experiment, cells were expanded in IL-2 (300 IU/ml) for 18 days, then washed as above and rested overnight in complete TexMACS medium. The following day, cells were stimulated for 20 minutes at 37° C. with medium alone or medium with IL-2 (300 IU/ml), IL-6 (100 ng/ml), or 130a1 G-CSF (100 ng/ml). Cytoplasmic and nuclear fractions were prepared and western blots performed as described in Example 39. Primary antibodies were obtained from Cell Signaling Technologies: Phospho-Stat3 (Tyr705) (D3A7) rabbit mAb #9145, Phospho-Stat5 (Tyr694) (C11C5) rabbit mAb #9359, and Histone H3 (96C10) mouse mAb #3638.

### Results

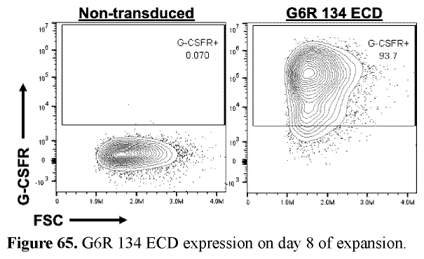

By flow cytometry (FIG. 65), a large proportion (93.7%) of CD3+ T cells transduced with the lentiviral vector encoding G6R 134 ECD expressed the G-CSFR ECD, whereas non-transduced T cells did not.

Thus, G6R 134 ECD can be expressed on the surface of engineered T cells.

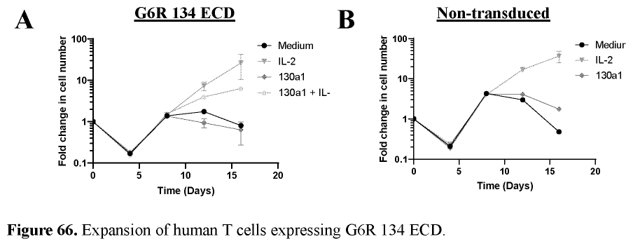

As shown in FIG. 66, T cells transduced with the lentiviral vector encoding G6R 134 ECD expanded well in medium containing IL-2, showed an intermediate level of expansion in response to IL-2+130a1 G-CSF, and poor expansion in response to 130a1 G-CSF alone (FIG. 66A). Non-transduced T cells expanded well in response to IL-2 but not 130a1 G-CSF (FIG. 66B). Neither culture expanded well in medium alone.

Thus, G6R 134 ECD does not appear to induce a growth signal in T cells, as assessed by this expansion assay.

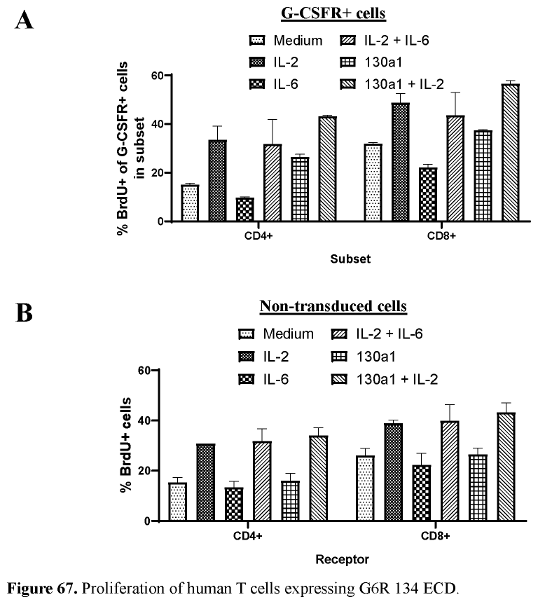

As shown in FIG. 67, CD4+ and CD8+ T cells expressing G6R 134 ECD demonstrated proliferation (BrdU incorporation) in response to IL-2, IL-2+IL-6, or 130a1 G-CSF+IL-2; 130a1 G-CSF induced modest proliferation of CD4+ T cells, and weak proliferation of CD8+ T cells (FIG. 67A). In contrast, non-transduced CD4+ and CD8+ T cells proliferated in response to IL-2, IL-2+IL-6, and 130a1 G-CSF+IL-2 but not in response to IL-6 or 130a1 G-CSF alone (FIG. 67B). None of the cultures proliferated in response to medium alone, with the exception of CD8+ T cells expressing G6R 134 ECD, which had elevated background proliferation in this particular experiment.

Thus, G6R 134 ECD appears to induce a weak or moderate proliferative signal in CD8+ and CD4+ T cells, respectively.

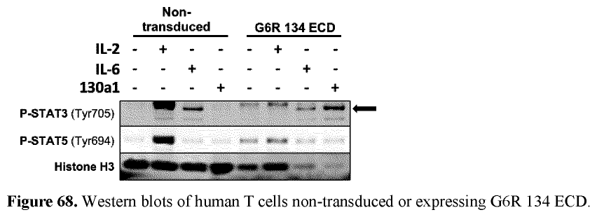

As shown in FIG. 68, in both non-transduced T cells and T cells expressing G6R 134 ECD, IL-2 induced the tyrosine phosphorylation of STAT3 (arrow) and STAT5, whereas IL-6 induced the tyrosine phosphorylation of STAT3 but not STAT5. In T cells expressing G6R 134 ECD, 130a1 G-CSF induced the tyrosine phosphorylation of STAT3 but not STAT5. 130a did not induce tyrosine phosphorylation of STAT3 or STAT5 in non-transduced T cells. Note that on the P-STAT3 image a dark, higher molecular band appears in the IL-2-stimulated conditions (especially in the non-transduced T cells). This is remnant signal from a previous probing of this membrane with phospho-STAT5 antibody. The lower molecular weight band (arrow) represents phospho-STAT3.

Thus, the hybrid intracellular domain of G6R 134 ECD induces a pattern of STAT phosphorylation similar to that induced by IL-6 in T cells.

### Example 41: Construction and Functional Evaluation of a Chimeric Receptor with a Variant Extracellular Domain from the G-CSFR Receptor and the Intracellular Domain of the EPO Receptor

We synthesized a chimeric receptor called GEPOR 134 ECD (FIG. 60) which contained the extracellular domain (134 variant) and transmembrane domain of human G-CSFR fused to the entire intracellular domain of human EPO receptor. The chimeric receptor subunit was encoded in a lentiviral vector which was used to transduce human CD4+ and CD8+ T cells. Transduced T cells (and non-transduced control T cells) were analyzed by flow cytometry to detect cell surface expression of GEPOR 134 ECD. The ability of GEPOR 134 ECD to induce T cell expansion was evaluated in vitro. Biochemical signaling events mediated by GEPOR 134 ECD were evaluated by western blot using phospho-specific antibodies against STAT5 and other signaling intermediaries.

### Methods

In general, we followed similar methods as described in Example 39; however, the experiments were adapted to focus on EPO-R and IL-2 signaling mechanisms.

Human T cells were engineered to express GEPOR 134 ECD and transduction efficiency was determined by flow cytometry on Day 8, as per Example 39.

T cells were expanded in IL-2 (300 IU/ml), 130a1 G-CSF (100 ng/ml), IL-2+130a1 G-CSF (300 IU/ml and 100 ng/ml, respectively) or medium alone (no added cytokine) for 16 days, with medium and the indicated cytokines refreshed every two days. For the non-transduced T cells, 307 G-CSF was added with 130a1 G-CSF to serve as a control for another arm of the experiment (not shown); use of this experimental convenience was justified by the fact that neither 130a1 G-CSF nor 307 G-CSF induces proliferation or other signaling events in non-transduced T cells. On days 4, 8, 12 and 16 of the expansion, T cells were counted using a Cytek™ Aurora flow cytometer.

For the western blot experiment, cells were expanded in IL-2 (300 IU/ml) for 18 days, then washed as above and rested overnight in complete TexMACS medium. The following day, cells were stimulated for 20 minutes at 37° C. with medium alone or medium with IL-2 (300 IU/ml) or 130a1 G-CSF (100 ng/ml). Cytoplasmic and nuclear fractions were prepared and western blots performed as described in Example 39. Primary antibodies were obtained from Cell Signaling Technologies: Phospho-Stat5 (Tyr694) (C11C5) rabbit mAb #9359, Histone H3 (96C10) mouse mAb #3638, Phospho-JAK2 (Tyr1007/1008) antibody #3771, Phospho-Akt (Ser473) (D9E) XP® rabbit mAb #4060, Phospho-p44/42 MAPK (Erk1/2) (Thr202/Tyr204) antibody #9101, and β-Actin (13E5) rabbit mAb #4970.

### Results

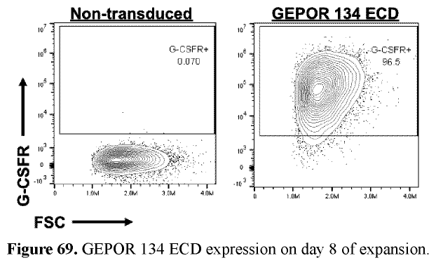

By flow cytometry (FIG. 69), a large proportion (96.5%) of CD3+ T cells transduced with the lentiviral vector encoding GEPOR 134 ECD expressed the G-CSFR ECD, whereas non-transduced T cells did not.

Thus, GEPOR 134 ECD can be expressed on the surface of engineered T cells.

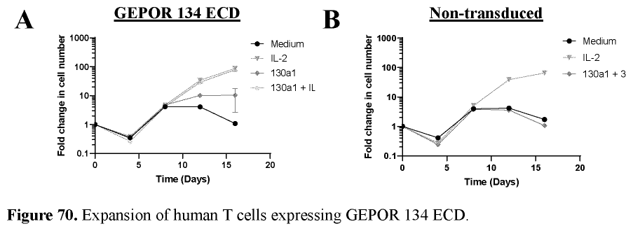

As shown in FIG. 70, T cells transduced with the lentiviral vector encoding GEPOR 134 ECD expanded well in medium containing IL-2 or IL-2+130a1 G-CSF and showed an intermediate level of expansion in response to 130a1 G-CSF alone (FIG. 64A). Non-transduced T cells expanded well in response to IL-2 but not 130a1 G-CSF+307 G-CSF (FIG. 70B). Neither culture expanded well in medium alone.

Thus, GEPOR 134 ECD appears to induce an intermediate growth signal in T cells, as assessed by this expansion assay.

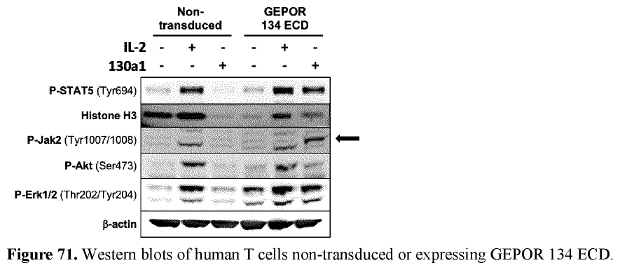

As shown in FIG. 71, in both non-transduced T cells and T cells expressing GEPOR 134 ECD, IL-2 induced the phosphorylation of STAT5, Akt and ERK1/2; the apparent phosphorylation of JAK2 in response to IL-2 is due to cross-reactivity of the anti-phospho-JAK2 antibody with phospho-JAK3 (note the difference in protein size compared to actual JAK2, indicated by the arrow). In T cells expressing GEPOR 134 ECD, 130a1 G-CSF induced the phosphorylation of STAT5, JAK2 (arrow), and ERK1/2 and weak phosphorylation of Akt. As expected, 130a1 G-CSF did not induce any biochemical signaling events in non-transduced T cells.

Thus, GEPOR 134 ECD induces biochemical signaling events that are similar to that induced by IL-2, but with weaker phosphorylation of Akt and with the expected activation of JAK2 in place of JAK3.

### Example 42: Construction and Functional Evaluation of a Chimeric Receptor with a Variant Extracellular Domain from the G-CSFR Receptor and the Intracellular Domain of Interferon Alpha Receptor 2

We synthesized a chimeric receptor called GIFNAR 134 ECD (FIG. 60) which contains the extracellular domain (134 variant), transmembrane domain, and Box 1 and 2 regions of human G-CSFR fused to a portion of the intracellular domain of human interferon receptor alpha 2. The chimeric receptor subunit was encoded in a lentiviral vector which was used to transduce human CD4+ and CD8+ T cells. Transduced T cells (and non-transduced control T cells) were analyzed by flow cytometry to detect cell surface expression of GIFNAR 134 ECD. The ability of GIFNAR 134 ECD to induce T cell expansion was evaluated in vitro. Biochemical signaling events mediated by GIFNAR 134 ECD were evaluated by western blot using phospho-specific antibodies against STAT1, STAT2 and other signaling intermediaries.

### Methods

In general, we followed similar methods as described in Example 39; however, the experiments were adapted to focus on IFN alpha signaling mechanisms.

Human T cells were engineered to express GIFNAR 134 ECD, and transduction efficiency was determined by flow cytometry on Day 8, as per Example 39.

T cells were expanded in IL-2 (300 IU/ml), 130a1 G-CSF (100 ng/ml), IL-2+130a1 G-CSF (300 IU/ml and 100 ng/ml, respectively) or medium alone (no added cytokine) for 16 days, with medium and the indicated cytokines refreshed every two days. On days 4, 8, 12 and 16 of the expansion, T cells were counted using a Cytek™ Aurora flow cytometer.

For the western blot experiment, cells were expanded in IL-2 (300 IU/ml) for 18 days, then washed as above and rested overnight in complete TexMACS medium. The following day, cells were stimulated for 20 minutes at 37° C. with medium alone or medium with IL-2 (300 IU/ml), IFNα (300 IU/ml) or 130a1 G-CSF (100 ng/ml). Cytoplasmic and nuclear fractions were prepared and western blots performed as described in Example 39. Primary antibodies were obtained from Cell Signaling Technologies: Phospho-Stat1 (Tyr70l) (58D6) rabbit mAb #9167, Phospho-Stat2 (Tyr690) (D3P2P) rabbit mAb #88410, Histone H3 (96C10) mouse mAb #3638, Phospho-JAK1 (Tyr1034/1035) (D7N4Z) rabbit mAb #74129, Phospho-Akt (Ser473) (D9E) XP® rabbit mAb #4060, Phospho-S6 Ribosomal Protein (Ser235/236) antibody #2211, and β-Actin (13E5) rabbit mAb #4970.

### Results

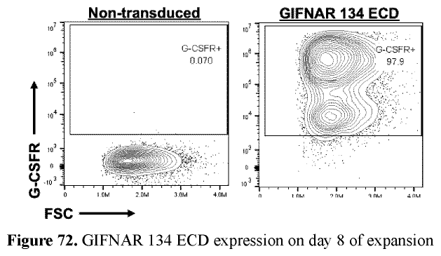

By flow cytometry (FIG. 72), a large proportion (97.9%) of CD3+ T cells transduced with the lentiviral vector encoding GIFNAR 134 ECD expressed the G-CSFR ECD, whereas non-transduced T cells did not.

Thus, GIFNAR 134 ECD can be expressed on the surface of engineered T cells.

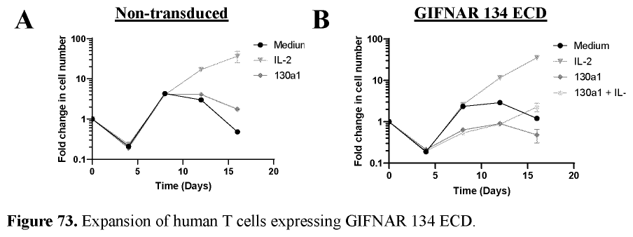

As shown in FIG. 73, T cells transduced with the lentiviral vector encoding GIFNAR 134 ECD expanded well in medium containing IL-2 but showed markedly reduced expansion in medium containing 130a1 G-CSF alone or IL-2+130a1 G-CSF; indeed, the level of expansion under these latter two conditions appeared to be inferior to that seen with medium alone (FIG. 73B). Non-transduced T cells expanded well in response to IL-2 but not 130a1 G-CSF (FIG. 73A). Neither culture expanded well in medium alone.

Thus, GIFNAR 134 ECD appears to induce a growth inhibitory signal in T cells, as assessed by this expansion assay.

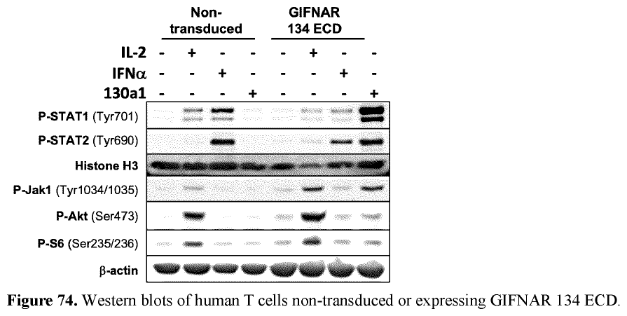

As shown in FIG. 74, in both non-transduced T cells and T cells expressing GIFNAR 134 ECD, IL-2 induced weak phosphorylation of STAT1 and typical levels of phosphorylation of JAK1, Akt and S6. In both non-transduced T cells and T cells expressing GIFNAR 134 ECD, IFNα induced strong phosphorylation of STAT1 and STAT2 with no apparent phosphorylation of JAK1, Akt or S6. In T cells expressing GIFNAR 134 ECD, 130a1 G-CSF induced strong phosphorylation of STAT1, STAT2 and JAK1 with no apparent phosphorylation of Akt or S6. 130a did not induce any biochemical signaling events in non-transduced T cells.

Thus, GIFNAR 134 ECD induces biochemical signaling events that are similar to those induced by IFNα.

### Example 43: Construction and Functional Evaluation of Chimeric Receptors with a Variant Extracellular Domain from the G-CSFR Receptor and Intracellular Signaling Domains from the Interferon Gamma Receptor

We synthesized two chimeric receptors called GIFNGR-1 307 ECD and GIFNGR-2 307 ECD (FIG. 60). GIFNGR-1 307 ECD contains the extracellular domain (307 variant) and transmembrane domain of human G-CSFR fused to the Box 1 and 2 regions of human IFNγR2 and the STAT1 binding site from the intracellular domain of human IFNγR1. GIFNGR-2 307 ECD contains the extracellular domain (307 variant), transmembrane domain, and Box 1 and 2 regions of human G-CSFR fused to the STAT1 binding site from the intracellular domain of human IFNγR1. The chimeric receptor subunits were encoded in separate lentiviral vectors, which were used to transduce human CD4+ and CD8+ T cells. Transduced T cells (and non-transduced control T cells) were analyzed by flow cytometry to detect cell surface expression of GIFNGR-1 307 ECD and GIFNGR-2 307 ECD. The ability of GIFNGR-1 307 ECD and GIFNGR-2 307 ECD to induce T cell expansion was evaluated in vitro. Biochemical signaling events mediated by GIFNGR-1 307 ECD and GIFNGR-2 307 ECD were evaluated by western blot using phospho-specific antibodies against STAT1 and other signaling intermediaries.

### Methods

In general, we followed similar methods as described in Example 39; however, the experiments were adapted to focus on IFN gamma signaling mechanisms.

Human T cells were transduced with lentiviral vectors encoding GIFNGR-1 307 ECD or GIFNGR-2 307 ECD. In addition, some T cells were co-transduced with lentiviral vectors encoding GIFNGR-1 307 ECD and G2R-3 134 ECD, or GIFNGR-2 307 ECD and G2R-3 134 ECD. G2R-3 134 ECD was tagged at the N-terminus with a Myc epitope (EQKLISEEDL) (SEQ ID NO.227), while GIFNGR-1 307 ECD and GIFNGR-2 307 ECD were tagged at the N-terminus with a Flag epitope (DYKDDDDK) (SEQ ID NO.228), which enabled the different receptor subunits to be distinguished by flow cytometry.

Transduction efficiency was determined by flow cytometry on Day 8, as per Example 39. The Flag and Myc tags were detected using BV421-conjugated and AlexaFluor™ 488-conjugated antibodies, respectively.

T cells were expanded in IL-2 (300 IU/ml), 130a1 G-CSF and/or 307 (100 ng/ml each), IL-2+130a1 G-CSF (300 IU/ml and 100 ng/ml, respectively), IL-2+307 G-CSF (300 IU/ml and 100 ng/ml, respectively) or medium alone (no added cytokine) for 18 days. Medium and the indicated cytokines were refreshed every two days. On days 4, 8, 12 and 16 of the expansion, T cells were counted using a Cytek™ Aurora flow cytometer.

For BrdU incorporation assays, T cells expanded in IL-2 were harvested on day 12 and washed twice in PBS and once in complete TexMACS medium. Cells were re-plated in fresh complete TexMACS medium containing medium alone or medium with IL-2 (300 IU/ml), IFNγ (100 ng/ml), IL-2+IFNγ (300 IU/ml and 100 ng/ml, respectively), 130a1 G-CSF (100 ng/ml), 307 G-CSF (100 ng/ml), 130a1 G-CSF+307 G-CSF (100 ng/ml each), 130a1 G-CSF+IFNγ (100 ng/ml each) or 307 G-CSF+IL-2 (100 ng/ml and 300 IU/ml, respectively). Cells were incubated for 48 hours 37° C., 5% CO2. The BrdU assay was performed as described in Example 39.

For the western blot experiment, T cells were expanded in IL-2 (300 IU/ml) for 18 days, washed as above, and rested overnight in complete TexMACS medium. The following day, cells were stimulated for 20 minutes at 37° C. with medium alone or medium with IL-2 (300 IU/ml), IFNγ (100 ng/ml) or 307 G-CSF (100 ng/ml). Cytoplasmic and nuclear fractions were prepared and western blots performed as described in Example 39. Primary antibodies were obtained from Cell Signaling Technologies: Phospho-Stat1 (Tyr701) (58D6) rabbit mAb #9167, Histone H3 (96C10) mouse mAb #3638, Phospho-JAK2 (Tyr1007/1008) antibody #3771, Phospho-Akt (Ser473) (D9E) XP® rabbit mAb #4060, Phospho-S6 Ribosomal Protein (Ser235/236) antibody #2211, and β-Actin (13E5) rabbit mAb #4970.

### Results

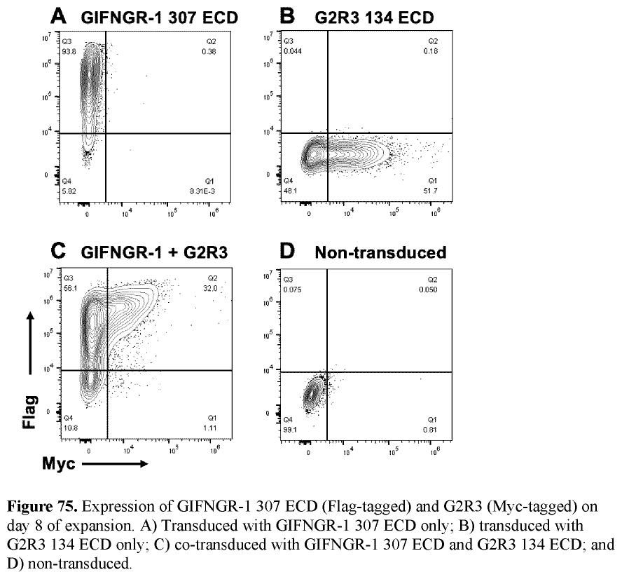

By flow cytometry (FIG. 75), a large proportion (93.8%) of CD3+ T cells transduced with the lentiviral vector encoding GIFNGR-1 307 ECD expressed the Flag epitope on the G-CSFR 307 ECD (FIG. 75A). Likewise, a large proportion (51.7%) of CD3+ T cells transduced with the lentiviral vector encoding G2R-3 134 ECD expressed the Myc epitope on the G-CSFR 134 ECD (FIG. 75B). For T cells that were co-transduced with lentiviral vectors encoding GIFNGR-1 307 ECD and G2R-3 134 ECD, 32.0% co-expressed both the Flag and Myc epitopes on the G-CSFR 307 and 134 ECDs, respectively (FIG. 69C). As expected, non-transduced T cells did not express either the Myc or Flag epitopes (FIG. 75D).

Thus, GIFNGR-1 307 ECD can be expressed on the surface of engineered T cells with or without G2R-3 134 ECD.

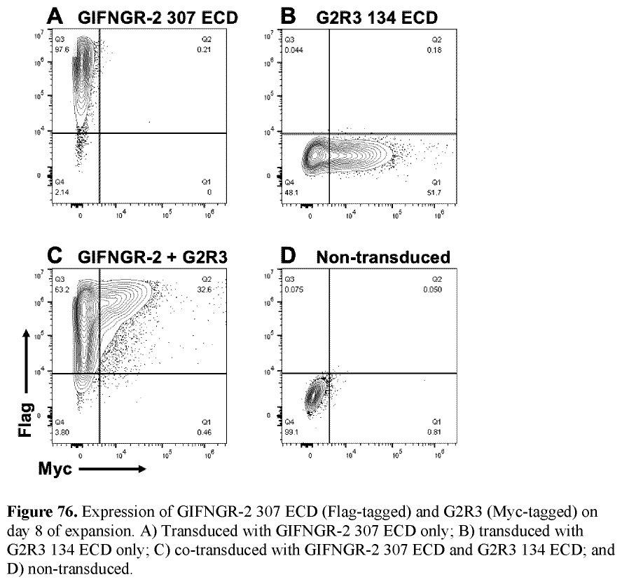

Similar flow cytometry results were obtained with GIFNGR-2 307 ECD (FIG. 76). A large proportion (97.6%) of CD3+ T cells transduced with the lentiviral vector encoding GIFNGR-2 307 ECD expressed the Flag epitope on the G-CSFR 307 ECD (FIG. 76A). Likewise, a large proportion (51.7%) of CD3+ T cells transduced with the lentiviral vector encoding G2R-3 134 ECD expressed the Myc epitope on the G-CSFR 134 ECD (FIG. 76B; this is the same data as in FIG. 75B). For T cells that were co-transduced with lentiviral vectors encoding GIFNGR-2 307 ECD and G2R-3 134 ECD, 32.6% co-expressed both the Flag and Myc epitopes on the G-CSFR 307 and 134 ECDs, respectively (FIG. 76C). As expected, non-transduced T cells did not express either the Myc or Flag epitopes (FIG. 76D; this is the same data as in FIG. 75D).

Thus, GIFNGR-2 307 ECD can be expressed on the surface of engineered T cells with or without G2R-3 134 ECD.

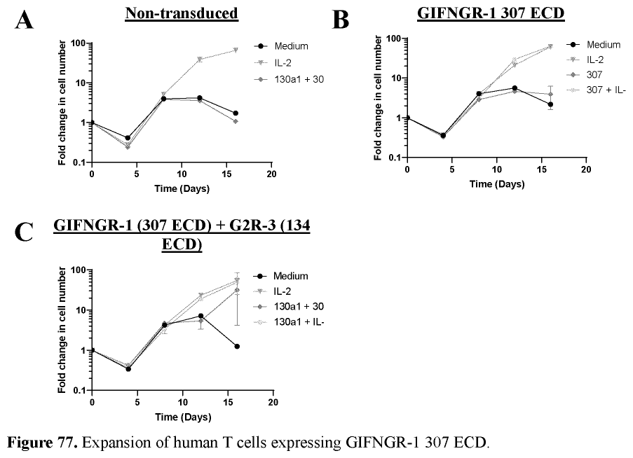

As shown in FIG. 71, T cells transduced with the lentiviral vector encoding GIFNGR-1 307 ECD expanded well in medium containing IL-2 or IL-2+307 G-CSF but not 307 G-CSF alone or medium alone (FIG. 77B). T cells co-transduced with lentiviral vectors encoding GIFNGR-1 307 ECD and G2R-3 134 ECD expanded well in medium containing IL-2 or IL-2+130a1 G-CSF (FIG. 77C). When these T cells were cultured in 130a1 G-CSF+307 G-CSF, they showed reduced expansion on day 12 which increased by day 16 (FIG. 77C). Non-transduced T cells expanded well in response to IL-2 but not 130a1 G-CSF+307 G-CSF (FIG. 77A). None of the T cell lines expanded well in medium alone.

Thus, GIFNGR-1 307 ECD appears to have a negligible effect on T cell growth, as assessed by this expansion assay.

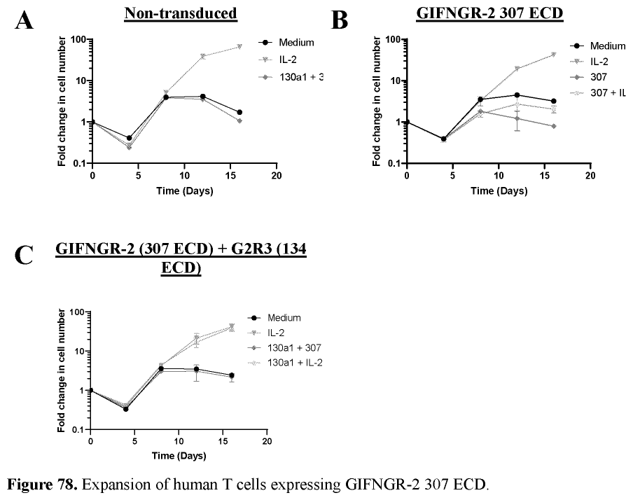

As shown in FIG. 78, T cells transduced with the lentiviral vector encoding GIFNGR-2 307 ECD expanded well in medium containing IL-2 but not in medium containing 307 G-CSF alone, IL-2+307 G-CSF, or medium alone (FIG. 78B). T cells co-transduced with lentiviral vectors encoding GIFNGR-2 307 ECD and G2R-3 134 ECD expanded well in medium containing IL-2 or IL-2+130a1 G-CSF (FIG. 78C). These T cells failed to expand when cultured in 130a1 G-CSF+307 G-CSF (FIG. 78C). Non-transduced T cells expanded well in response to IL-2 but not 130a1 G-CSF+307 G-CSF (FIG. 78A, which shows the same data as FIG. 77A). None of the T cell lines expanded well in medium alone.

Thus, GIFNGR-2 307 ECD appears to inhibit T cell growth, as assessed by this expansion assay.

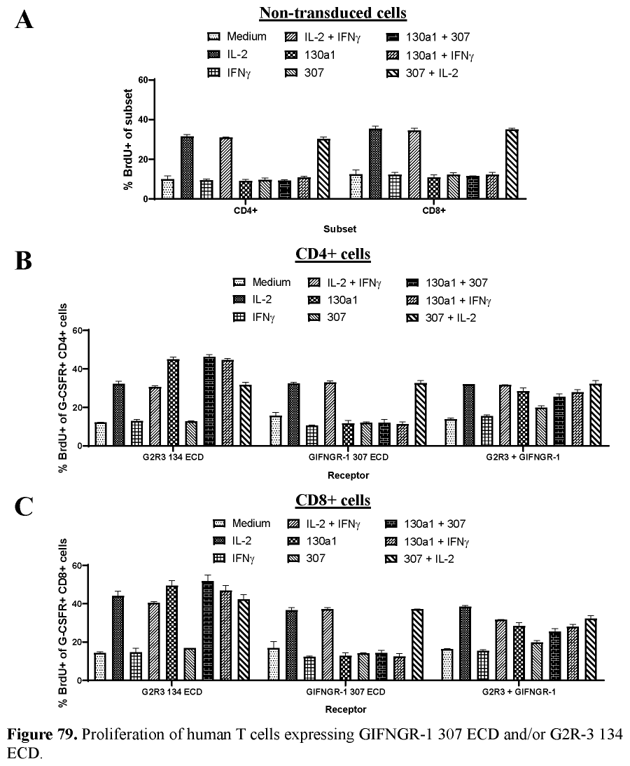

As shown in FIG. 79, non-transduced CD4+ and CD8+ T cells demonstrated proliferation (BrdU incorporation) in response to IL-2, IL-2+IFNγ, and 307 G-CSF+IL-2 (FIG. 79A).

CD4+ and CD8+ T cells expressing G2R-3 134 ECD alone demonstrated proliferation in response to IL-2, IL-2+IFNγ, 130a1 G-CSF, 130a1 G-CSF+307 G-CSF, 130a1 G-CSF+IFNγ, and 307 G-CSF+IL-2 (FIG. 79B, C).

CD4+ and CD8+ T cells expressing GIFNGR-1 307 ECD alone demonstrated proliferation in response to IL-2, IL-2+IFNγ, and 307 G-CSF+IL-2 (FIG. 79B, C).

CD4+ and CD8+ T cells co-expressing GIFNGR-1 307 ECD and G2R-3 134 ECD demonstrated proliferation in response to IL-2, IL-2+IFNγ, 130a1 G-CSF, 130a1 G-CSF+307 G-CSF, 130a1 G-CSF+IFNγ, and 307 G-CSF+IL-2 (FIG. 79B, C). They also showed weak proliferation in response to 307 G-CSF alone (FIG. 79B, C).

Thus, GIFNGR-1 307 ECD appears to have a neutral or negligible effect on CD8+ and CD4+ T cell proliferation.

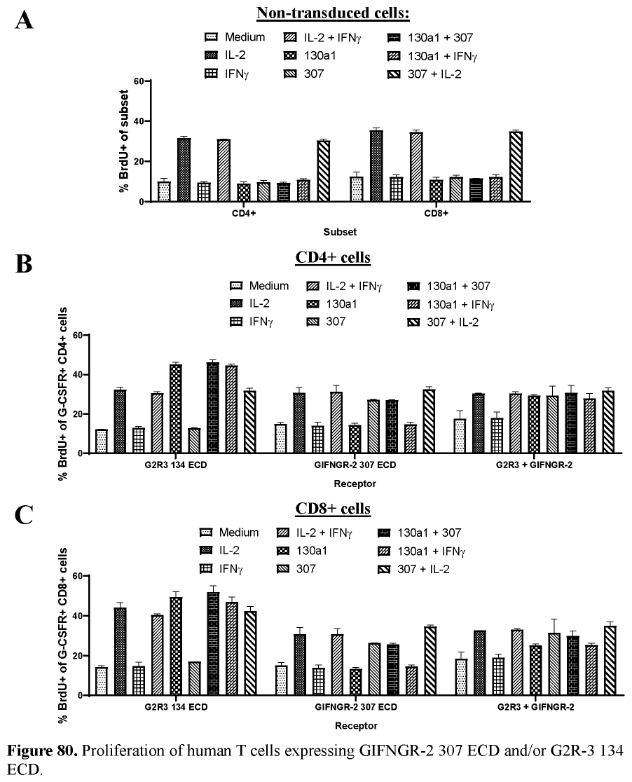

FIG. 80 shows the proliferation results for GIFNGR-2. The data for non-transduced T cells and CD4+ and CD8+ T cells expressing G2R-3 134 ECD alone are the same as shown in FIG. 79 and hence are not described again.

CD4+ and CD8+ T cells expressing GIFNGR-2 307 ECD alone demonstrated proliferation in response to IL-2, IL-2+IFNγ, 307 G-CSF, 130a1 G-CSF+307 G-CSF, and 307 G-CSF+IL-2 (FIG. 80B, C).

CD4+ and CD8+ T cells co-expressing GIFNGR-2 307 ECD and G2R-3 134 ECD demonstrated proliferation in response to IL-2, IL-2+IFNγ, 130a1 G-CSF, 307, 130a1 G-CSF+307 G-CSF, 130a1 G-CSF+IFNγ, and 307 G-CSF+IL-2 (FIG. 80B, C).

Thus, despite inhibiting T cell expansion in long-term culture (FIG. 78), GIFNGR-2 307 ECD appears to enhance CD4+ and CD8+ T cell proliferation, as assessed by BrdU assay.

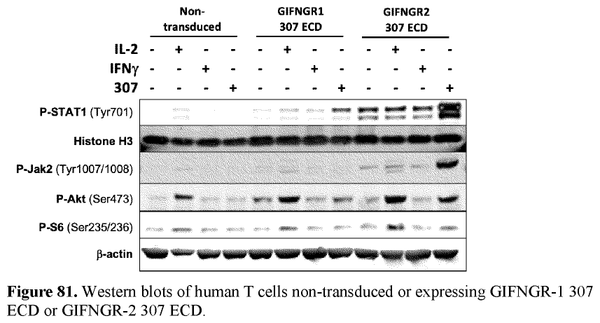

As shown in FIG. 81, in both non-transduced T cells and T cells expressing GIFNGR-1 307 ECD or GIFNGR-2 307 ECD, IL-2 induced weak/negligible phosphorylation of STAT1, strong phosphorylation of Akt, and (in this particular experiment) weak phosphorylation of S6. IFNγ did not appear to induce any of these signaling events. In T cells expressing GIFNGR-1 307 ECD or GIFNGR-2 307 ECD, 307 G-CSF induced strong phosphorylation of STAT1. In T cells expressing GIFNGR-2 307 ECD, 307 G-CSF also induced strong phosphorylation of JAK2 and Akt.

Thus, GIFNGR-1 307 ECD and GIFNGR-2 307 ECD induce tyrosine phosphorylation of STAT1 but differ in other ways from the native IFNγ receptor signal.

### Example 44: Construction and Functional Evaluation of Bicistronic Constructs Encoding the Chimeric Receptor G12/2R-1 134 ECD or G2R-3 134 ECD and a Mesothelin-Specific Chimeric Antigen Receptor (CAR)

We synthesized bicistronic constructs called CAR_T2A_G2R-3-134-ECD, G2R-3-134-ECD_T2A_CAR, CAR_T2A_G12/2R-1-134-ECD, and G12/2R-1-134-ECD_T2A_CAR, as well as a monocistronic construct that drives expression of mesothelin CAR alone. The nomenclature of bicistronic constructs indicates how the gene segments are ordered from the 5′ to 3′ end. For example, CAR_T2A_G2R-3-134-ECD encodes (from 5′ to 3′) a mesothelin-specific CAR, a T2A ribosomal skip element, and G2R-3 134 ECD. The bicistronic constructs were expressed using a single lentiviral vector encoding the mesothelin CAR and either G2R-3 134 ECD or G12/2R-1 ECD, separated by a T2A ribosomal skip element. These bicistronic constructs were used to transduce human CD4+ and CD8+ T cells, and transduced T cells (and non-transduced control T cells) were analyzed by flow cytometry to detect cell surface expression of the G-CSFR ECD and the CAR. The ability of 130a1 G-CSF to induce T cell proliferation through G2R-3 134 ECD or G12/2R-1 134 ECD was evaluated in vitro. T cells were also evaluated by intracellular flow cytometry to measure expression of the cytokines IFNγ, TNFα, and IL-2, as well as the cell surface markers CD69 and CD137 in response to activation of the CAR by co-incubation of T cells with the mesothelin-positive human ovarian cancer cell line OVCAR3.

### Methods

In general, we followed similar methods as described in Example 31; however, the experiments were adapted to focus on co-expression and functionality of the mesothelin CAR and the engineered chimeric cytokine receptors G2R-3 134 ECD and G12/2R-1 134 ECD, as described below.

For flow cytometry to detect the G-CSFR ECD and mesothelin CAR, cells were expanded in IL-7 and IL-15 (10 ng/ml each) for 8 days. Cells were incubated with antibodies specific for CD3 (BV750-conjugated), CD4 (PE-conjugated), CD8 (PerCP-conjugated) and G-CSFR ECD (APC-conjugated), as well as recombinant Fc-tagged mesothelin protein (FITC-conjugated) and efluor506™ viability dye.

For intracellular flow cytometry, transduced T cells were expanded in 130a1 G-CSF for 13 days. OVCAR3 cells (ATCC) were grown in ATCC-formulated RPMI-1640 medium containing 0.01 mg/ml bovine insulin and 20% fetal bovine serum. T cells were washed and replated in complete TexMACS medium in the presence or absence of OVCAR3 cells (at a 2:1 effector:target cell ratio) for 13 hours at 37 degrees Celsius, 5% CO2. The last four hours of incubation were performed in the presence of a protein transport inhibitor (Brefeldin A). At the conclusion of incubation, the cells were stained with Zombie NIR™ Fixable Viability Kit, followed by a cocktail of antibodies against CD3 (BV510-conjugated), CD4 (AlexaFluor™ 700-conjugated), G-CSFR (APC-conjugated), CD69 (BV785-conjugated) and CD137 (BV605-conjugated), as well as recombinant mesoethelin (FITC-conjugated) and Fc block. The cells were then fixed and permeabilized (BD Cytofix/Cytoperm™ set) and stained for the cytokines IFNγ (BV650-conjugated), TNFα (PECy7-conjugated) and IL-2 (PE-CF594-conjugated). Cells were analyzed using a Cytek Aurora flow cytometer.

### Results

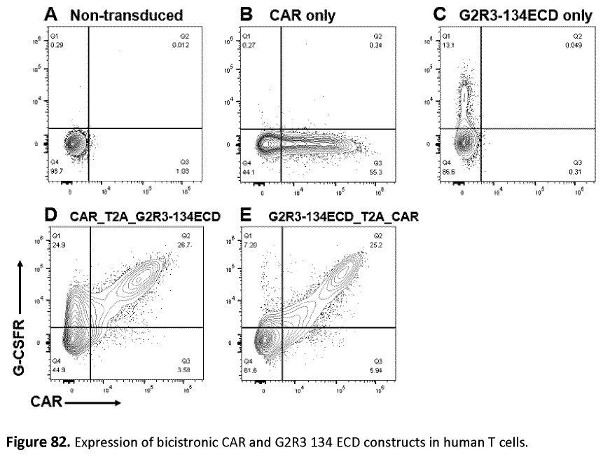

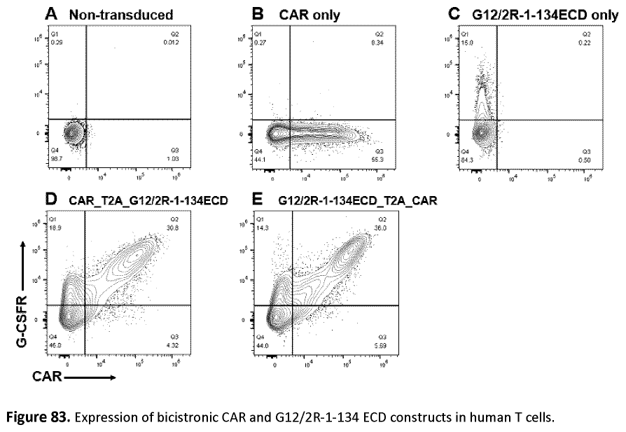

By flow cytometry (FIGS. 82 and 83), non-transduced cells exhibited negligible expression of G-CSFR ECD and mesothelin CAR (FIGS. 66A and 67A), whereas 55.3% of CD3+ T cells transduced with a lentivirus encoding the mesothelin CAR construct alone expressed the CAR (FIGS. 82B and 83B), and 13.1% of T cells transduced with a lentivirus encoding G2R-3 134 ECD expressed the G-CSFR ECD (FIG. 82C). 26.7% of T cells transduced with a lentivirus encoding CAR_T2A_G2R-3-134ECD expressed both the G-CSFR ECD and mesothelin CAR (FIG. 82D), while 25.2% of CD3+ T cells transduced with a lentivirus encoding G2R-3-134ECD_T2A_CAR expressed both the G-CSFR ECD and mesothelin CAR (FIG. 82E). Likewise, 13.1% of T cells transduced with a lentivirus encoding G12/2R-1 134 ECD alone expressed the G-CSFR ECD (FIG. 83C). 30.8% of T cells transduced with a lentivirus encoding CAR_T2A_G12/2R-1-134ECD expressed both the G-CSFR ECD and mesothelin CAR (FIG. 83D), and 36.0% of T cells transduced with a lentivirus encoding G12/2R-1-134ECD_T2A_CAR expressed the both the G-CSFR ECD and mesothelin CAR (FIG. 83E).

Thus, bicistronic lentiviral vectors encoding a mesothelin CAR plus G2R-3 134 ECD or mesothelin CAR plus G12/2R-1 134 ECD can cause cell surface expression of both the CAR and chimeric cytokine receptor.

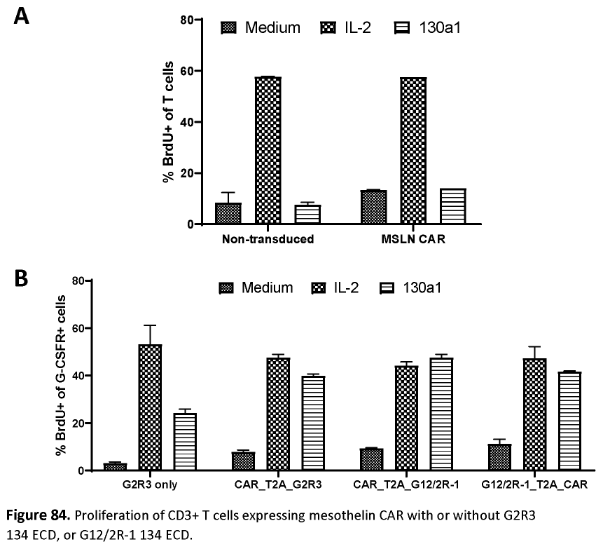

By BrdU incorporation assay (FIG. 84), non-transduced T cells or T cells engineered to express the mesothelin CAR alone proliferated in response to IL-2, with negligible proliferation in response to 130a1 G-CSF or medium alone. T cells expressing G2R-3 134 ECD alone proliferated in response to IL-2 or 130a1 G-CSF (FIG. 84B). T cells transduced with lentiviral vectors encoding CAR_T2A_G2R-3-134ECD, CAR_T2A_G12/2R-1-134ECD, or G12/2R-1-134ECD_T2A_CAR proliferated in response to IL-2 or 130a1 G-CSF. None of the T cell populations showed significant proliferation in response to medium alone.

Thus, G2R-3 134 ECD and G12/2R-1 134 ECD retain their functional properties when co-expressed as part of a bicistronic construct with a mesothelin CAR, irrespective of whether they are arranged upstream or downstream of the CAR.

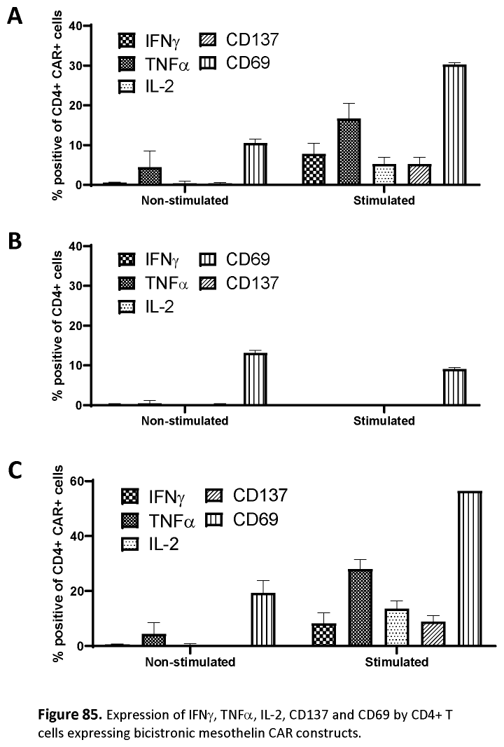

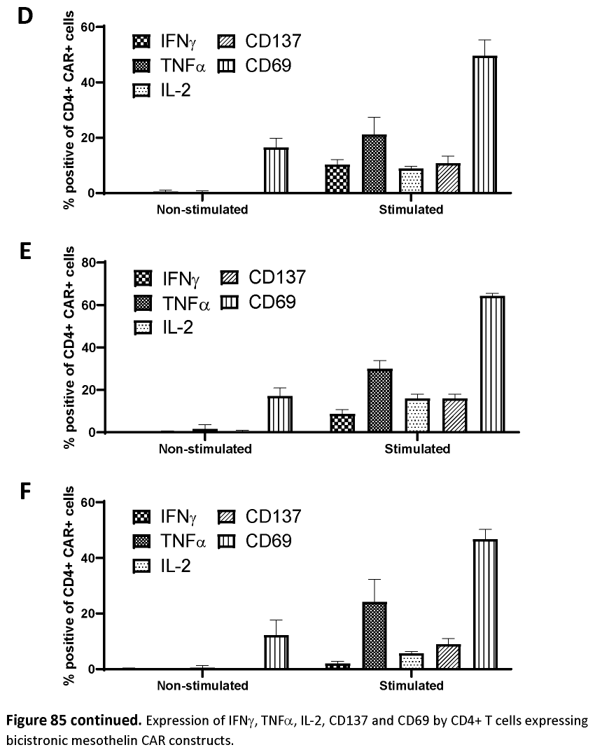

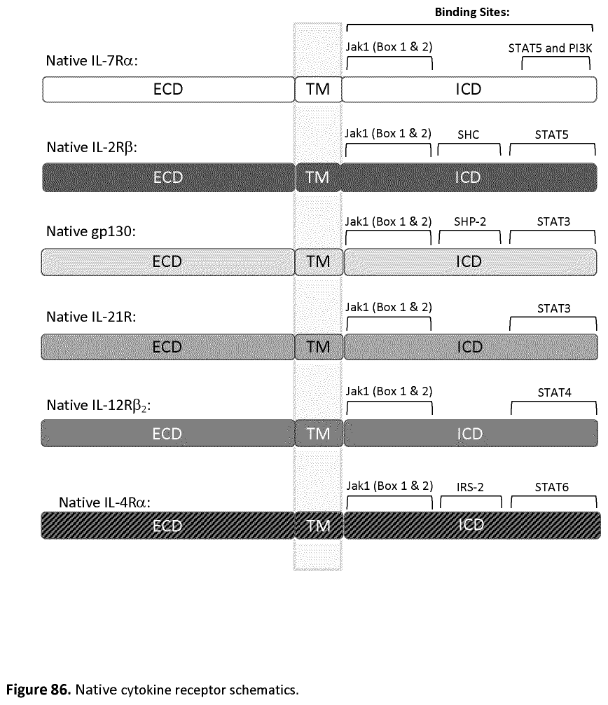

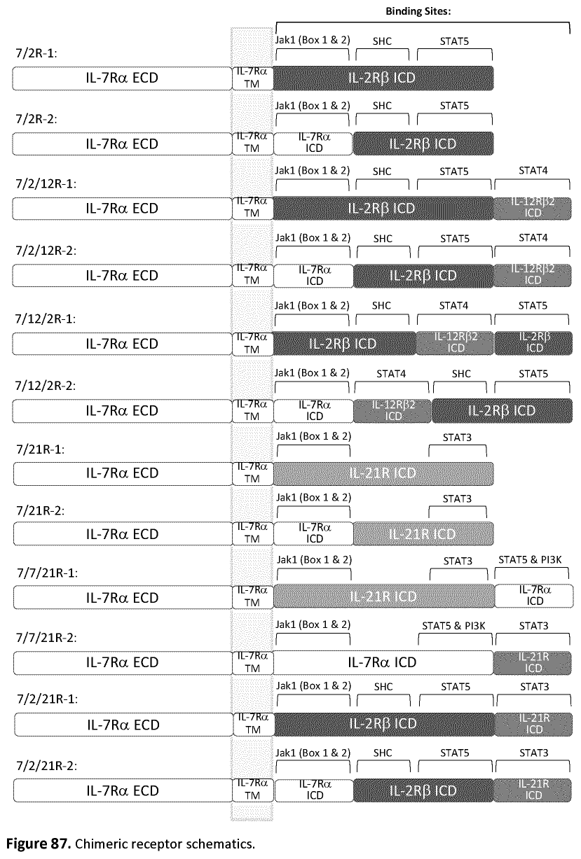

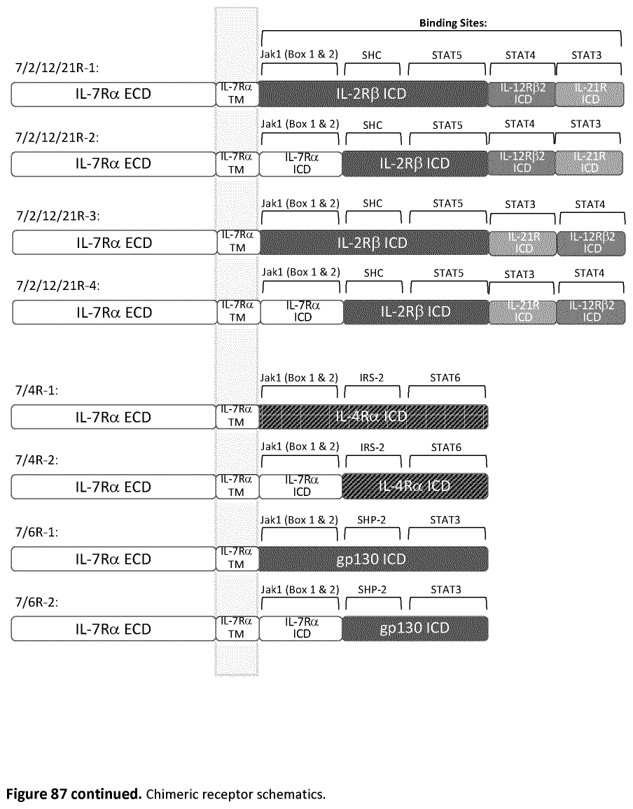

By intracellular flow cytometry (FIG. 85), T cells expressing the monocistronic mesothelin CAR construct upregulated expression of the cytokines IFNγ, TNFα and IL-2, and the activation markers CD69 and CD137, in response to stimulation with OVCAR3 cells (FIG. 85A). In contrast, there was no observed change in expression of IFNγ, TNFα, IL-2, CD69 or CD137 in T cells engineered to express G12/2R-1 134 ECD alone and stimulated with OVCAR3 cells (FIG. 85B). T cells expressing the bicistronic construct CAR_T2A_G2R-3-134ECD showed increased expression of IFNγ, TNFα, IL-2, CD69 and CD137 in response to OVCAR3 cells (FIG. 85C). Likewise, T cells expressing the G2R-3-134ECD_T2A_CAR, CAR_T2A_G12/2R-1-134ECD or G12/2R-1-134ECD_T2A_CAR constructs showed increased expression of these cytokines and markers in response to stimulation with OVCAR3 cells (FIGS. 85D, 85E and 85F).

Thus, the mesothelin CAR retains its functional properties when expressed as part of a bicistronic lentiviral construct with G2R-3 134 ECD or G12/2R-1 134 ECD, as evidenced by upregulation of IFNγ, TNFα and IL-2 and the activation markers CD69 and CD137 in response to stimulation with cognate antigen on OVCAR3 cells. The CAR was functional when arranged either upstream or downstream of G2R-3 134 ECD or G12/2R-1 134 ECD.

### Example 45: Human T Cells Co-Expressing a Mesothelin-Specific CAR and an IL-2 Emulating Chimeric Cytokine Receptor Demonstrate Cytotoxicity Against Mesothelin-Expressing Target Cells

Experiments are conducted to show that CAR constructs and chimeric cytokine receptors, when co-expressed, exhibit their expected functional properties and that stimulation of chimeric cytokine receptors such as G2R-3 134 ECD enhances CAR-mediated cytotoxity against target cells expressing the appropriate CAR antigen. The following example uses a mesothelin-specific CAR co-expressed with G2R-3 134 ECD.

### Methods

A mammalian expression transfer plasmid encoding firefly luciferase, pCCL Luc Puromycin is introduced into a lentiviral vector using a standard calcium phosphate transfection protocol and 2^{nd} generation packaging vectors (AddGene). The mesothelin-expressing OVCAR3 cell line is transduced with the luciferase-encoding lentivirus and selected over 2 weeks for resistance to puromycin (1.5 μg/ml, Sigma, Cat #P8833) to generate a puromycin-resistant cell line for use as a target cell population in cytotoxicity assays.

Luciferase-expressing target OVCAR3 lines are seeded at 2×10⁴ cells, 100 μl per well in a 96-well plate (Corning, Cat #3917) and incubated overnight to allow adherence to the plate. Human PBMC-derived T cells are lentivirally transduced to express a mesothelin-specific CAR alone or with G2R-3 134 ECD. T cells are plated (100 μl per well) in a 96-well plate and split over a 2-fold dilution series to achieve a range of effector-to-target (E:T) ratios from 20:1 to 0.625:1. The diluted T cell suspensions are then be added to the adherent tumor cell cultures to achieve a total volume of 200 μl. IL-2 (300 IU/ml), 130a1 G-CSF (100 ng/ml) or PBS are added to some wells to determine whether cytotoxicity is enhanced by IL-2 or 130a1 G-CSF. All conditions are tested in triplicate. Three wells of target cells alone and 3 wells of media alone are plated to determine the maximal and minimal relative luminescence units (RLU) for the assay. Cells are cultured for 24-48 h at 37° C. in 5% CO₂.

The day of the assay, 22 μl 10× stock of Xenolight™ D-Luciferin (Perkin Elmer, Cat #122799) are added to each well, and plates are incubated for 10 min at RT in the dark. Plates are scanned on a luminescence plate reader (Perkin Elmer Wallac Envision 2104 Multilabel Reader). RLU from triplicate wells are averaged, and the percent specific cytotoxicity is determined by the following equation: percent specific cytotoxicity=100×(Max Luminescence RLU−Test Luminescence RLU)/(Min Luminescence RLU−Max Luminescence RLU). In addition, target cells are imaged for morphological signs of cell death at 24 & 48 hours.

### Expected Results

T cells expressing the mesothelin-specific CAR cause lysis of target cells in a dose-dependent manner. Target cell lysis is increased by the addition of IL-2, as reflected by increased killing at lower E:T ratios. For T cells co-expressing the mesothelin-specific CAR with G2R-3 134 ECD, target cell lysis is increased with the addition of 130a1 G-CSF to a similar extent as seen with IL-2. These results show that, in T cells co-expressing a mesothelin-specific CAR with G2R-3 134 ECD, the CAR mediates target cell recognition and killing as expected and that the cytotoxicity of T cells can be selectively enhanced by stimulation of G2R-3 134 ECD with 130a1 G-CSF.

### Example 46: Construction and Functional Evaluation of a Controlled Paracrine Signaling System Paired with a CAR

Using the methods described in Example 41, a CPS system is paired with a CAR, using the example of G12/2R 134 ECD, IL-18 and a mesothelin-specific CAR (see construct called CAR_P2A_G12-2R1-134_T2A_hIL-18 in Table 31). This combined system is functionally assessed in vitro and in vivo.

### Methods

The three cDNAs are separated by P2A and T2A sites to allow three proteins to be produced from a single mRNA transcript. With such tricistronic constructs, it is not uncommon for one cDNA to be expressed at higher levels than the other, and the optimal configuration needs to be determined empirically. Therefore, at least three configurations are constructed (listed from 5′ to 3′): (a) G12/2R-1 134 ECD, IL-18, Meso CAR; (b) IL-18, G12/2R-1 134 ECD, Meso CAR; and (c) Meso CAR G12/2R-1 134 ECD, IL-18 (FIGS. 82-85) (Table 31).

T cell transduction, expansion, proliferation, IL-18 secretion and biochemical signaling will be assessed using similar methods as Example 49. CAR expression and recognition and killing of mesothelin-positive tumor cells will be assessed using similar methods as Example 44.

### Expected Results

T cells are transduced with two different lentiviral constructs: one encoding G12/2R-1 134 ECD in tandem with IL-18 and Meso CAR (referred to in this example as CPS^{2/12+18}+Meso CAR; also referred to as CAR_P2A_G12-2R1-134_T2A_hIL-18 in Table 31), and the other encoding G12/2R-1 134 ECD and Meso CAR (Table 31) (to serve as a control).

By flow cytometry, T cells expressing CPS^{2/12+18}+Meso CAR or G12/2R-1+Meso CAR demonstrate co-expression of the human G-CSFR ECD and Meso CAR on a significant proportion of T cells (15-90%). Meso CAR-expressing T cells upregulate expression of activation markers such as CD69 or CD137 when exposed to target cells expressing mesothelin, similar to Example 44.

By BrdU assay, Meso CAR-expressing T cells proliferate in response to target cells expressing mesothelin.

By IFN-γ ELISA, Meso CAR-expressing T cells secrete IFN-γ in response to target cells expressing mesothelin. IFN-γ secretion is enhanced by addition of IL-2, IL-2+IL-12, and/or 130a1 G-CSF to cultures. The effect of 130a1 G-CSF is more pronounced in cells expressing CPS^{2/12+18}+Meso CAR, as IL-18 can enhance IFN-γ secretion by T cells, especially when combined with IL-2 and IL-12 signals.

By in vitro cytotoxicity assay (similar to Example 45), Meso CAR-expressing T cells kill target cells expressing mesothelin. This effect is enhanced by addition of IL-2, IL-2+IL-12, and/or 130a1 G-CSF to cultures, since both IL-2 and IL-12 can enhance the cytotoxicity of T cells. The effect of 130a1 G-CSF is more pronounced in cells expressing CPS^{2/12+18}+Meso CAR, as IL-18 can further enhance the cytotoxicity of T cells when combined with IL-2 and IL-12 signals.

In in vivo tumor regression models, Meso CAR-expressing T cells induce regression of tumor cells expressing mesothelin. This effect is enhanced by administration of IL-2 or 130a1 G-CSF to cultures, since IL-2 can enhance the cytotoxicity of T cells. (IL-12 is too toxic to safely administer systemically.) 130a1 G-CSF has a stronger anti-tumor effect than IL-2, as the combined IL-2+IL-12 signal from G12/2R 134 ECD should be stronger than IL-2 alone. The effect of 130a1 G-CSF is even more pronounced in cells expressing CPS^{2/12+18}+Meso CAR, as IL-18 further enhance the anti-tumor activity of T cells when combined with IL-2 and IL-12 signals.

By methods such as flow cytometry and immunohistology, T cells expressing CPS^{2/12+18}+Meso CAR (in comparison to T cells expressing G12/2R-1+Meso CAR) induce a stronger anti-tumor immune response. This is reflected by increased infiltration of tumors by activated host T cells, NK cells, myeloid cells and other cell types due to the paracrine effects of IL-18 in the tumor microenvironment.

Thus, the experiments in this example demonstrate superior effector function of T cells expressing CPS^{2/12+18} over T cells expressing G12/2R-1 alone due to the stimulatory effects of IL-18 on such functions as IFN-γ secretion and cytotoxicity by T cells, and paracrine effects on host immune cells.

### Example 47: Construction and Functional Evaluation of a Dual-Cytokine Controlled Paracrine Signaling System

Chronic signaling can have undesirable or deleterious effects on cells. For example, prolonged or chronic exposure to an IL-12 signal could promote the terminal differentiation of T cells, thereby limiting their proliferative potential and/or persistence. Therefore, in some circumstances it may be desirable to use a CPS system in which, for example, the IL-2 and IL-12 signals are controlled by distinct, orthogonal cytokines. In this manner, T cells are induced to proliferate in response to one cytokine (for example, via stimulation of G2R-3 with 130a1 G-CSF to emulate an IL-2 signal) and to differentiate at a later time in response to another cytokine (for example, via stimulation of G12R-1 with 307 G-CSF to emulate an IL-12 signal). To enable such control, methods described in the above examples are used to construct and evaluate a CPS system comprised of a tricistronic vector encoding G2R-3 134 ECD, G12R-1 307 ECD and IL-18 (referred to as CPS²⁺¹²⁺¹⁸). This combined system is functionally assessed in vitro and in vivo.

### Example 48: Construction and Functional Evaluation of Controlled Paracrine Signaling Systems that Emulate Other Cytokine Combinations

Using the methods described in Example 41, other cytokine combinations are emulated by substituting G12/2R-1 with chimeric cytokine receptors encoding other ICDs, such as those shown in FIG. 31. These chimeric receptors could use the 134 ECD of human G-CSFR, or other variant ECDs of human G-CSFR (e.g., 307 ECD), or ECDs from other cytokine receptors.

In place of the IL-18 cDNA, cDNAs encoding other cytokines or chemokines are inserted, such as CXCL13, IL-21, CCL3, CCL4, CD40 ligand, B cell activating factor (BAFF), Flt3 ligand, CCL21, CCL5, XCL1, CCL19, IFN-γ, IFN-α, IL-17, or TNF-α. Other cell surface proteins are inserted, such as the receptor NKG2D (Table 30). Human, murine or other versions of such factors are used, depending on the intended application.

### Example 49: Construction and Expression of a Chimeric Receptor with Extracellular and Transmembrane Domains from the IL-7 Alpha Receptor Subunit and Intracellular Domain from the IL-2/IL-15 Receptor Beta Subunit

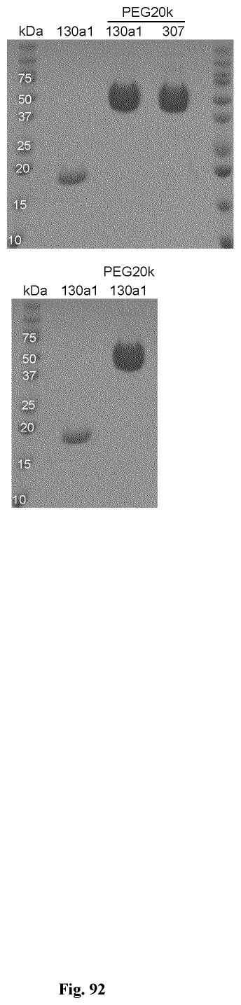

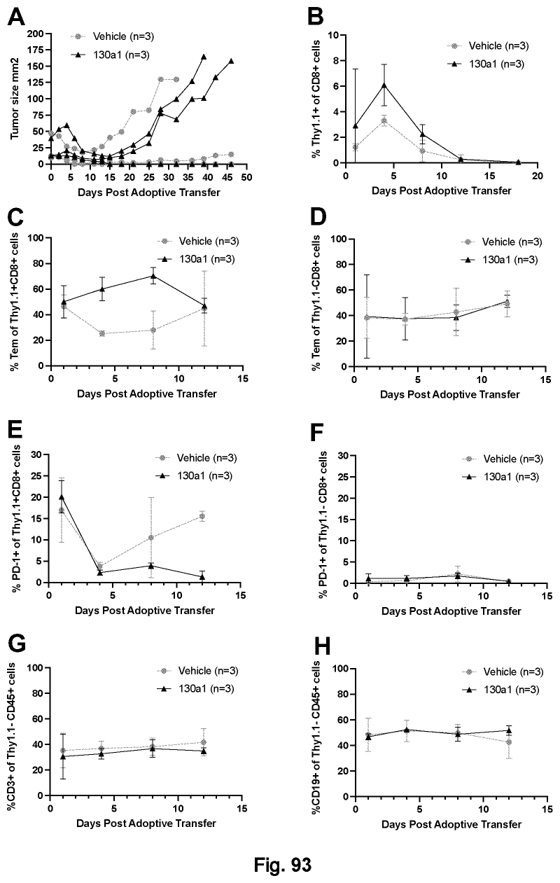

We synthesized a chimeric receptor called 7/2R-1 (FIGS. 92 and 93) which contained the extracellular and transmembrane domains of the human IL-7 receptor alpha subunit fused to the intracellular domain of the human IL-2/IL-15 receptor beta subunit. The IL-7 receptor alpha subunit was tagged at its N-terminus with a standard Flag epitope (DYKDDDDK) (SEQ ID NO.228) to enable discrimination of this chimeric receptor subunit from the native IL-7 receptor alpha subunit, which is also frequently expressed by T cells. The chimeric receptor subunit was encoded in a lentiviral vector which was used to transduce human CD4+ and CD8⁺ T cells. Twelve days later, transduced cells (and non-transduced control cells) were analyzed by flow cytometry to detect expression of the chimeric receptor subunit relative to the native IL-7 receptor alpha subunit.

### Methods

The Flag-tagged chimeric receptor (7/2R-1) was synthesized and cloned into a lentiviral transfer plasmid (Twist Bioscience). The transfer plasmid and lentiviral packaging plasmids (psPAX2, pMD2.G, Addgene) were co-transfected into the lentiviral packaging cell line HEK293T/17 (ATCC) as follows. Cells were plated overnight in Opti-MEM™ I Reduced Serum Medium, GlutaMAX™ Supplement (Gibco™) containing 5% fetal bovine serum and 0.2 mM sodium pyruvate. Plasmid DNA and Lipofectamine™ 3000 Transfection Reagent (Gibco™) were mixed, according to the manufacturer's specifications, and added dropwise onto cells. The cells were incubated for 6 hours at 37° C., 5% CO₂, followed by replacement of the medium and further incubation overnight. The following day, the cellular supernatant was collected from the plates, stored at 4° C., and medium replaced. The following day, cellular supernatant was collected from the cells, and pooled with supernatant from the previous day. The supernatant was centrifuged briefly, to remove debris, and filtered through a 0.45 μm filter. The supernatant was spun for 90 minutes at 25,000 rpm using a SW-32Ti rotor in a Beckman Optima L-XP Ultracentrifuge. The supernatant was removed, and the pellet was resuspended via 30 minutes of gentle shaking in a suitable volume of Opti-MEM I medium.

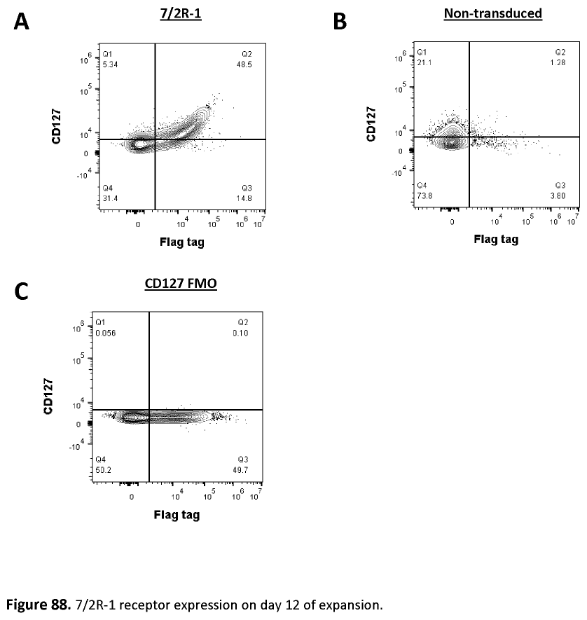

Primary human T cells were isolated from a healthy donor leukapheresis product as follows. Peripheral blood mononuclear cells (PBMCs) were isolated using density gradient centrifugation. CD4+ and CD8+ T cells were isolated using human CD4 and CD8 MicroBeads (Miltenyi Biotec) following manufacturer's specifications. For some experiments, T cells were viably frozen and thawed at a later time point. Isolated T cells were plated at 5×10⁵ per well in a 48-well plate in TexMACS™ medium (Miltenyi Biotec) containing 3% human serum (Sigma), and 0.5% gentamicin (DIN 0226853) (hereafter referred to as “complete TexMACS”). Human T cell TransAct™ (Miltenyi Biotec) was added (10 μl per well), and cells were incubated at 37° C., 5% CO₂. Twenty to 28 hours following activation, the T cells were transduced with the lentivirus encoding the receptor constructs. Twenty-four hours after transduction, fresh medium was added to T cells, along with IL-7 (10 ng/ml), IL-7 and IL-15 (10 ng/ml each), or no cytokine, and the cells were incubated for a further 48 hours. On day 12 of expansion, receptor expression was assessed by flow cytometry as follows. Cells were incubated for 15 minutes at 4° C. with an anti-human CD127 eFluor™ 450-conjugated antibody (1:50 dilution), anti-Flag antibody conjugated to PE or PECy7 (1:50 dilution), anti-human CD3 antibody conjugated to BV750™ (1:50 dilution), anti-human CD4 antibody conjugated to PE or AlexaFluor™ 700 (1:25 dilution), anti-human CD8 antibody conjugated to PerCP (1:100), and eBioscience™ Fixable Viability Dye eFluor™ 506 (1:1000 dilution). Cells were then washed twice in buffer and analyzed on a Cytek™ Aurora flow cytometer. FIG. 88 shows the results for CD3+ T cells, which include both CD4+ and CD8+ subsets.

### Results

A large proportion (48.5%) of CD3+ T cells transduced with the lentiviral vector encoding 7/2R-1 co-expressed CD127 (IL-7Rα) and the Flag epitope tag (FIG. 88A), whereas non-transduced T cells showed low/moderate expression of native CD127 but not the Flag epitope tag (FIG. 88B). The specificity of CD127 detection was confirmed by the CD127 FMO control (FIG. 88C).

Thus, 7/2R-1 can be expressed on the surface of lentivirally transduced T cells.

### Example 50: Expansion of Human T Cells Transduced with 7/2R-1 and Stimulated with IL-7

To determine whether 7/2R-1 promoted T cell expansion (increase in cell number), T cells from the transduced and non-transduced conditions from Example 49 were cultured in medium alone versus IL-7+IL-15 or versus IL-7 and counted at regular intervals. The combination of IL-7+IL-15 was chosen as a comparator to IL-7 alone, as IL-7+IL-15 are commonly used to expand human T cells after lentiviral transduction.

### Methods

Human T cells were isolated and transduced to express 7/2R-1 as described in Example 49 (day 0). The cells were expanded in IL-7 (10 ng/ml), IL-7 and IL-15 (10 ng/ml each) or medium only for a total of 16 days, with medium and cytokine refreshed every two days. On days 4, 8, 12 and 16 of expansion, live T cells were counted using a Cytek™ Aurora flow cytometer to assess fold expansion relative to day 0.

### Results

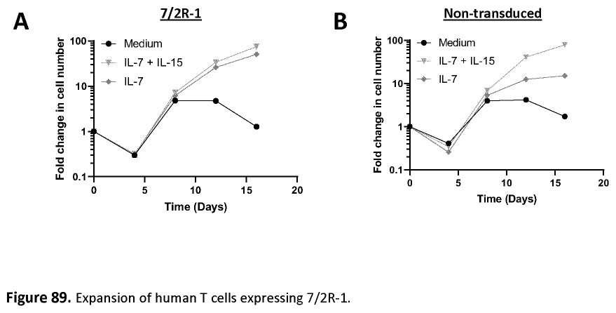

As shown in FIG. 57, CD3+ T cells transduced with the lentiviral vector encoding 7/2R-1 showed equivalent expansion in either IL-7 or IL-7+IL-15 (FIG. 89A), whereas non-transduced CD3+ T cells expanded well in IL-7+IL-15 but only modestly in IL-7 (FIG. 89B). Neither culture expanded well in medium alone.

Thus, expression of 7/2R-1 on T cells leads to increased expansion in response to IL-7, reaching a cell density equivalent to that induced by IL-7+IL-15. This provides functional evidence that the intracellular domain of the IL-2/IL-15 receptor beta subunit is functional in the context of the 7/2R-1 chimeric receptor.

### Example 51: Proliferation of Human T Cells Transduced with 7/2R-1 and Stimulated with IL-7 Assessed by BrdU Incorporation Assay

To determine whether 7/2R-1 promoted cell proliferation (cell cycling), T cells from the transduced and non-transduced conditions from Example 49 were cultured in medium alone versus medium containing IL-2, IL-7 or the combination of IL-2+IL-7. Cell proliferation was measured by a 48-hour BrdU assay.

### Methods

Cells were isolated and engineered to express 7/2R-1 as described in Example 49. Cells expressing 7/2R-1 were expanded in IL-7 (10 ng/ml) and non-transduced cells were expanded in IL-7 and IL-15 (10 ng/ml each). On day 12 of expansion, cells were harvested and washed twice in PBS, and once in complete TexMACS medium, and re-plated in fresh complete TexMACS medium containing the relevant assay cytokine (IL-7 at 10 ng/ml, IL-2 at 300 IU/ml, IL-7 and IL-2 at 10 ng/ml and 300 IU/ml, respectively, or no cytokine) for 48 hours at 37° C., 5% CO2. The BrdU assay procedure followed the instruction manual for the BD Pharmingen™ FITC BrdU Flow Kit (557891), with the following addition: surface staining was performed, as described in Example 49, to assess 7/2R-1 expression in addition to BrdU incorporation. Flow cytometry was performed using a Cytek™ Aurora instrument.

### Results

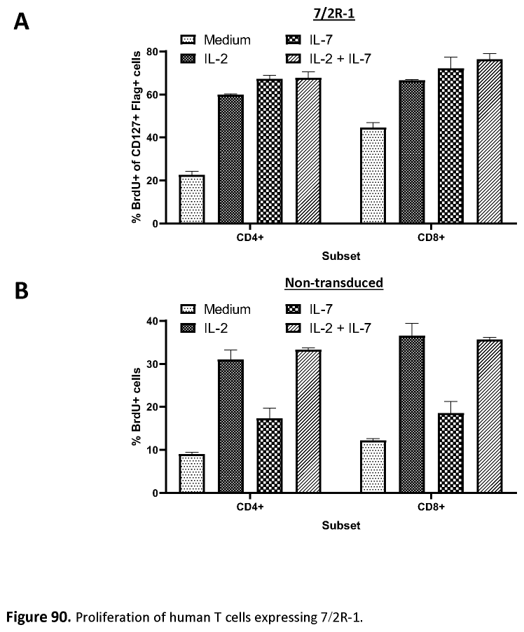

As shown in FIG. 58, CD4+ and CD8+ T cells transduced with the lentiviral vector encoding 7/2R-1 showed equivalent proliferation in response to either IL-2, IL-7 or IL-2+IL-7 (FIG. 90A), whereas non-transduced CD4+ and CD8+ T cells proliferated well in response to IL-2 and IL-2+IL-7 but only modestly in response to IL-7 (FIG. 90B). None of the cultures proliferated well in medium alone, with the exception of CD8+ T cells expressing 7/2R-1, which had high background proliferation in this particular assay.

Thus, expression of 7/2R-1 on T cells leads to increased proliferation in response to IL-7, reaching levels equivalent to that induced by IL-2 or IL-2+IL-7. This provides functional evidence that the intracellular domain of the IL-2/IL-15 receptor beta subunit is functional in the context of the 7/2R-1 chimeric receptor.

### Example 52: Western Blot Detection of Biochemical Signaling Events in Human T Cells Expressing 7/2R-1

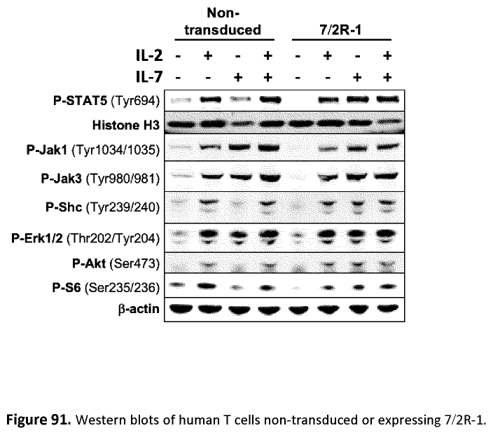

To determine whether 7/2R-1 induced similar biochemical signaling events as the native IL-2 receptor, T cells from the transduced and non-transduced conditions from Example 31 were cultured in medium alone versus medium containing IL-2, IL-7 or the combination of IL-2+IL-7. Cell lysates were prepared and probed by western blot with antibodies against various proteins involved in IL-7 and IL-2 receptor signaling (FIG. 91).

### Methods

Cells were isolated and engineered to express 7/2R-1 as described in Example 49. Cells expressing 7/2R-1 were expanded in IL-7 (10 ng/ml) and non-transduced cells were expanded in IL-7 and IL-15 (10 ng/ml each). On day 12 of expansion, cells were harvested, washed twice PBS, washed once in complete TexMACS medium, and rested overnight in complete TexMACS medium containing no added cytokine at 37° C., 5% CO₂.

To perform western blots, cells were stimulated with no cytokine, IL-2 (300 IU/ml), IL-7 (10 ng/ml), or IL-2 and IL-7 (at 300 IU/ml and 10 ng/ml, respectively) for 20 minutes at 37° C. Cells were washed once in a buffer containing 10 mM HEPES, pH 7.9, 1 mM MgCl₂, 0.05 mM EGTA, 0.5 mM EDTA, pH 8.0, 1 mM DTT, and 1× Pierce Protease and Phosphatase Inhibitor Mini Tablets (A32961). Cells were lysed in the wash buffer above, with the addition of 0.2% NP-40 substitute (Sigma), for 10 minutes on ice and centrifuged for 10 minutes at 13,000 rpm at 4° C., after which supernatant (cytoplasmic fraction) was collected. The nuclear pellet was resuspended and extracted in the above wash buffer with the addition of 0.42M NaCl and 20% glycerol. Nuclear pellets were extracted for 30 minutes on ice, with frequent vortexing, and centrifuged for 20 minutes at 13,000 rpm at 4° C., after which the soluble nuclear fraction (supernatant) was collected. The cytoplasmic and nuclear fractions were heated (70° C.) in reducing buffer for 10 minutes and run on NuPAGE™ 4-12% Bis-Tris Protein Gels. The gels were transferred to nitrocellulose membrane (60 min at 20V in a Trans-Blot® SD Semi-Dry Transfer Cell), dried, and blocked for 1 hr in Intercept® Blocking Buffer in TBS (927-50000, LI-COR). The blots were incubated with the indicated primary antibodies (1:1,000) overnight at 4° C. in Intercept® Blocking Buffer in TBS containing 0.1% Tween20. The primary antibodies utilized were obtained from Cell Signaling Technologies: Phospho-Shc (Tyr239/240) antibody #2434, Phospho-Akt (Ser473) (D9E) XP® rabbit monoclonal antibody #4060, Phospho-S6 Ribosomal Protein (Ser235/236) antibody #2211, Phospho-p44/42 MAPK (Erk1/2) (Thr202/Tyr204) antibody #9101, β-Actin (13E5) rabbit monoclonal antibody #4970, Phospho-JAK1 (Tyr1034/1035) (D7N4Z) rabbit monoclonal antibody #74129, Phospho-JAK3 (Tyr980/981) (D44E3) rabbit monoclonal antibody #5031, Phospho-Stat5 (Tyr694) (C11C5) rabbit monoclonal antibody #9359, and Histone H3 (96C10) mouse monoclonal antibody #3638. Blots were washed three times in TBS containing 0.1% Tween20 and incubated for 30-60 minutes at room temperature with secondary antibodies (1:10,000) in TBS buffer containing 0.1% Tween20. The secondary antibodies were obtained from Cell Signaling Technologies: anti-mouse IgG (H+L) (DyLight™ 800 4× PEG Conjugate) #5257 and anti-rabbit IgG (H+L) (DyLight™ 800 4× PEG Conjugate) #5151. Blots were washed and exposed on a LI-COR Odyssey imager.

### Results

As shown in FIG. 59, in both non-transduced T cells and T cells expressing 7/2R-1, IL-2 induced the phosphorylation of STAT5, JAK1, JAK3, Shc, Erk1/2, Akt, and S6. A similar pattern was seen in response to IL-2+IL-7 in both T cell cultures. In non-transduced T cells, IL-7 stimulation induced less phosphorylation of STAT5, Shc, Akt and S6 compared to IL-2 stimulation. In contrast, in T cells expressing 7/2R-1, the phosphorylation patterns induced by IL-7 and IL-2 were indistinguishable. Histone H3 and beta-actin served as loading controls.

Thus, stimulation of 7/2R-1 by IL-7 results in similar biochemical signaling events in T cells as seen after stimulation of the native IL-2 receptor, providing biochemical evidence that the intracellular domain of the IL-2/IL-15 receptor beta subunit was functional in the context of the 7/2R-1 chimeric receptor.

### Example 53: Construction and Evaluation of an Alternative Chimeric Receptor Containing the Extracellular and Transmembrane Domains of IL-7 Receptor Alpha Domain and the Intracellular Domain of the IL-2 Receptor Beta Subunit

We have designed an alternative version of 7/2R-1, which we refer to as 7/2R-2 (FIGS. 92 and 93). The key difference is that the fusion site between IL-7R alpha and IL-2R beta has been moved such that 7/2R-2 contains the Box 1 and 2 regions of IL-7Rα rather than IL-2R3. This receptor may be advantageous if, for example, the fusion site in 7/2R-1 proves to be immunogenic in humans. Following the methods in Examples 49-52, a Flag epitope tagged version of 7/2-R2 is constructed and encoded in a lentiviral vector, and this lentiviral vector is transduced into T cells. Expression of 7/2R-2 is assessed by flow cytometry; the functional properties of 7/2R-2 are assessed by expansion and proliferation assays, and the biochemical signaling properties of 7/2R-2 are assessed by western blot.

### Example 54: Chemical Addition of a PEG20k Moiety to the N-Terminus of 130a1 G-CSF

We synthesized a modified protein called PEG20K_G-CSF_130a1 (FIG. 93) (also referred to as PEG-130a1 G-CSF) that includes the full sequence of 130a1 G-CSF with a 20 kDa polyethylene glycol (PEG) moiety chemically linked to the N-terminus of the protein. In addition, we synthesized an analogous version of 307 G-CSF, which we refer to as PEG20K_G-CSF_307 or PEG-307 G-CSF.

### Methods

20 mg of purified 130a1 G-CSF was combined with 44 mg of 20 kDa methoxy-polyethylene glycol propionaldehyde (mPEG-ALD, Creative PEGworks). Reaction buffer (10 mM Sodium acetate pH 5.0, 5 mM NaCl) was added to a final volume of 2 ml. The PEGylation reaction was initiated by adding 40 μl of a 1.0 M solution of sodium cyanoborohydride (Final [NaBH₃CN]=20 mM) and mixing thoroughly. The reaction was then incubated on a rotator at 18° C. for 8 hours. The reaction was terminated by adding 3 ml of termination buffer (10 mM Sodium acetate pH 3.5, 5 mM NaCl). The final 5 ml sample was then run on a cation exchange column to separate mono-PEGylated protein from any remaining unmodified protein or multi-PEGylated species. The same protocol was successfully completed with 307 G-CSF.

### Results

SDS-PAGE showed a homogenous population of mono-PEGylated 130a1 G-CSF and 307 G-CSF, which showed similar signalling activity to unmodified protein in vitro.

### Example 55: In a Murine Mammary Carcinoma Model, Treatment with 130a1 G-CSF Selectively Enhances the Expansion and Anti-Tumor Phenotype of Tumor-Specific T Cells Expressing G4R-1 134 ECD

The variant cytokine 130a1 G-CSF was tested for the ability to enhance the expansion, phenotype and anti-tumor activity of T cells engineered to express G4R 134 ECD in an in vivo murine breast cancer model.

### Methods

We used similar methods as described in Example 36, with the exception that Thy1.1+ OT-I cells were retrovirally transduced to express G4R 134 ECD instead of G2R-3 134 ECD. The antibody panel used for flow cytometry of blood samples included: murine CD45 (Clone 30-F11, APC-conjugated), CD3 (Clone 17A2, AF700-conjugated), CD8a (Clone 53-6.7, PerCP-conjugated), CD4 (Clone RM4-5, BV510-conjugated), NK1.1 (Clone PK136, PECy7-conjugated), CD19 (Clone 6D5, SparkNIR685-conjugated), CD11b (Clone M1/70, FITC-conjugated), CD11c (Clone N418, BV711-conjugated), Ly6C (Clone HK1.4, BV605-conjugated), and Ly6G (Clone 1A8, PE-conjugated). Flow cytometry was performed on a Cytek Aurora instrument.

### Results

All mice received an equivalent dose (5×10⁶) of Thy1.1+ OT-I cells transduced to express G4R 134 ECD. The transduction efficiency was 74.7%, as assessed by the percentage of Thy1.1+ cells expressing human G-CSFR+ by flow cytometry on Day 0.

In the 130a1 G-CSF-treated group, 1/3 mice experienced complete regression (CR) of their tumor and remained tumor-free until day 46, when the study was stopped for practical reasons (FIG. 93A). Similarly, in the vehicle-treated group, 1/3 mice experienced CR. Thus, the 130a1 G-CSF- and vehicle-treated groups did not show statistically significant difference.

The expansion and persistence of Thy1.1+ OT-I cells were monitored by flow cytometry in serial blood samples collected from mice. In the 130a1 G-CSF-treated group, Thy1.1+ OT-I cells reached a mean peak engraftment of 6.1% of all CD8⁺ T cells on Day 4 compared to a peak of 3.3% for the vehicle-treated group (FIG. 93B).

The phenotype of Thy1.1+ OT-I cells was monitored by flow cytometry of serial blood samples. In the 130a1 G-CSF-treated group, the majority (mean=70.4%) of Thy1.1+OT-I cells adopted a T effector memory (Tem) phenotype (CD44+CD62L−) by Day 8 compared to a mean of 27.9% for the vehicle-treated group (FIG. 93C). In contrast, host (Thy1.1−) CD8⁺ T cells showed no significant difference in the percentage of Tem cells between the 130a1 G-CSF- and vehicle-treated groups at any time point (FIG. 93D).

Expression of the Programmed Death-1 (PD-1) molecule was assessed by flow cytometry. In the 130a1 G-CSF-treated group, only a small minority (mean=1.3%) of Thy1.1+ OT-I cells expressed PD-1 by Day 12 compared to a mean of 15.5% for the vehicle-treated group (FIG. 93E). In contrast, host (Thy1.1−) CD8⁺ T cells showed no significant difference in the percentage of PD-1+ cells between the 130a1 G-CSF- and vehicle-treated groups at any time point (FIG. 93F).

Flow cytometry demonstrated that the overall percentage of host (Thy1.1−) CD4⁺ T cells and CD19+ B cells (relative to host CD45+ cells) did not differ significantly between the 130a1 G-CSF- and vehicle-treated groups at any time point (FIG. 93G,H). Likewise, treatment with 130a1 G-CSF did not affect neutrophil, eosinophil or monocyte counts in peripheral blood (data not shown).

Throughout the study, all mice in both groups maintained a normal body weight and exhibited good general health, despite tumor progression in some.

Thus, 130a1 G-CSF was able to induce modest yet selective expansion of tumor-specific CD8⁺ T cells expressing G4R 134 ECD. This resulted in a 33% durable CR rate. Treatment with 130a1 G-CSF induced a significant proportion of tumor-specific CD8⁺ T cells to adopt a Tem phenotype and to decrease expression of PD-1. There were no overt toxicities associated with 130a1 G-CSF treatment.

### Example 56: In a Cell Proliferation Assay, Pegylated Versions of G-CSF 130a1 and 307 Show Similar Potency and Selectivity Compared to Non-Pegylated Versions

An in vitro cell proliferation experiment was performed to compare pegylated versus non-pegylated versions of G-CSF (wildtype, 130a1 and 307) for the ability to stimulate proliferation of cells expressing G2R-3 134 ECD or the wildtype human G-CSF receptor.

### Methods

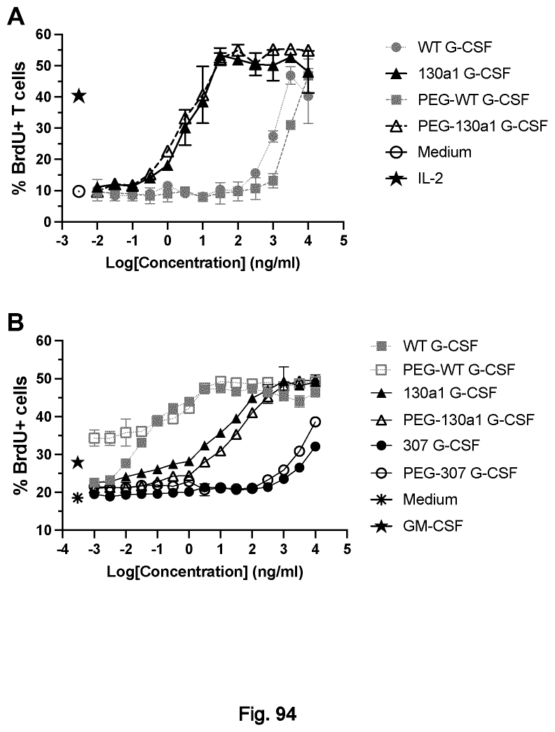

In FIG. 94A, human CD4 and CD8 T cells derived from PBMC of a healthy donor were lentivirally transduced to express G2R-3 134 ECD using methods similar to Example 35. To assess proliferation (BrdU incorporation), T cells were washed twice in PBS and once in medium and then re-plated in fresh medium containing the indicated cytokines: human IL-2 (Proleukin, 300 IU/ml, positive control), medium alone (negative control), or the indicated concentrations (in ng/ml) of wildtype G-CSF, pegylated wildtype G-CSF (PEG-WT G-CSF), 130a1 G-CSF, or PEG-130a1 G-CSF. Cells were cultured for 48 hours. The BrdU assay procedure followed the instruction manual for the BD Pharmingen™ FITC BrdU Flow Kit (557891), with one exception: cells were also stained to detect cell surface expression of the G-CSFR ECD (as described in Example 35). Flow cytometry was performed using a Cytek Aurora instrument. BrdU incorporation is shown for T cells positive for expression of G-CSFR.

In FIG. 94B, a similar experiment was performed using the human myeloid cell line OCI-AML-1 which naturally expresses the wildtype human G-CSF receptor and proliferates in response to wildtype G-CSF (See Example 34). Cells were washed twice in PBS and once in medium and then re-plated in medium containing the indicated cytokines: human GM-CSF (20 ng/ml, positive control), medium alone (negative control), or the indicated concentrations (in ng/ml) of wildtype G-CSF, PEG-WT G-CSF, 130a1 G-CSF, PEG-130a1 G-CSF, 307 G-CSF, or PEG-307 G-CSF. Cells were cultured for 48 hours and assessed for BrdU incorporation by flow cytometry.

### Results

As shown in FIG. 94A, human T cells expressing G2R-3 134 ECD showed a similar proliferative response to pegylated versus non-pegylated 130a1 G-CSF across a range of cytokine concentrations. These cells also showed a similar proliferative response to pegylated versus non-pegylated wildtype G-CSF; however, the response was significantly weaker than that seen with 130a1 versions of G-CSF.

As shown in FIG. 94B, human OCI-AML1 myeloid cells showed a similar proliferative response to pegylated versus non-pegylated 130a1 G-CSF across a range of concentrations; the only noted difference was that slightly higher concentrations of pegylated 130a1 G-CSF were required to induce the same level of cell proliferation. OCI-AML1 cells showed a similar proliferative response to pegylated versus non-pegylated 307 G-CSF. Finally, OCI-AML1 cells showed a similar proliferative response to pegylated versus non-pegylated wildtype G-CSF; at lower concentrations, the response to pegylated G-CSF was greater than that to non-pegylated G-CSF. OCI-AML1 cells showed a much stronger response to wildtype versions of G-CSF than to 130a1 or 307 versions of G-CSF.

Thus, pegylation of 130a1 G-CSF had negligible effects on the ability to induce cell proliferation through the G2R-3 134 ECD receptor. Moreover, pegylation of 130a1 G-CSF or 307 G-CSF had negligible effects on the specificity of these cytokines for G2R-3 134 ECD versus the wildtype G-CSF receptor.

### Example 57: Pegylated and Non-Pegylated Versions of 130a1 G-CSF Induce Similar Biochemical Signaling Events in T Cells

An in vitro western blotting experiment was performed to compare pegylated versus non-pegylated 130a1 G-CSF for the ability to induce biochemical signaling events in human T cells expressing G2R-3 134 ECD.

### Methods

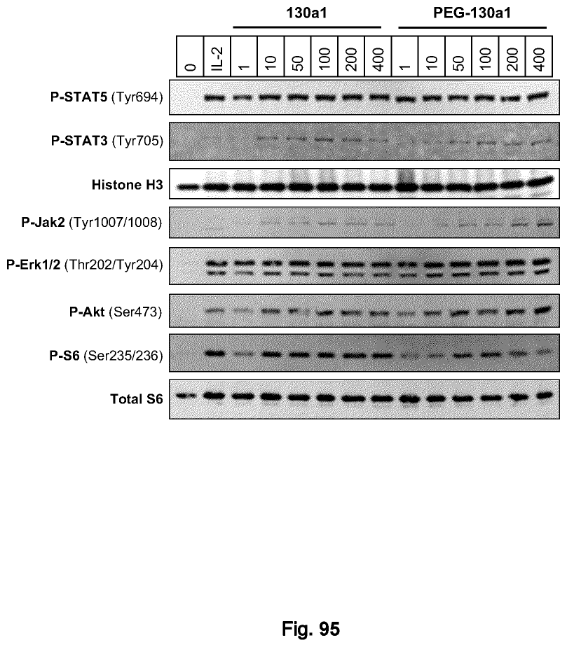

Human PBMC-derived T cells expressing G2R-3 134 ECD were prepared similar to Example 39 and then stimulated for 20 min with medium alone; human IL-2 (Proleukin, 300 IU/ml); or non-pegylated or pegylated versions of 130a1 G-CSF ranging from 1 to 400 ng/ml. Similar to Example 39, cellular and nuclear extracts were prepared and subjected to western blotting using the antibodies indicated in FIG. 95 and described in detail in prior examples.

### Results

As shown in FIG. 95, both the pegylated and non-pegylated versions of 130a1 G-CSF induced signaling events similar to IL-2, including phosphorylation of STAT5, STAT3, Erk-1/2, Akt and S6. Unlike IL-2, pegylated and non-pegylated 130a1 G-CSF induced phosphorylation of JAK2, which is expected based on their design. Pegylated 130a1 G-CSF appeared slightly less potent than non-pegylated 130a1 G-CSF, as evidenced by slightly diminished phosphorylation events at 1 ng/ml.

Thus, the pegylated and non-pegylated versions of 130a1 G-CSF induce similar biochemical signaling events in human T cells and with only minor differences in potency.

### Example 58: In a Murine Mammary Carcinoma Model, Treatment with Pegylated G-CSF 130a1 Selectively Enhances the Expansion and Anti-Tumor Activity of Tumor-Specific T Cells Expressing G2R-3 134 ECD

The pegylated form of the variant cytokine 130a1 G-CSF was tested across three dosing schedules for the ability to enhance the expansion, phenotype and anti-tumor activity of T cells expressing G2R-3 134 ECD in a murine breast cancer model.

### Methods

We used similar methods as described in Example 36. All mice received an equivalent dose (5×10⁶) of Thy1.1+ OT-I cells transduced to express G2R-3 134 ECD. The antibody panel used for flow cytometry of blood samples was the same as in Example 59.

### Results

The transduction efficiency was 79.8%, as assessed by the percentage of Thy1.1+ cells expressing human G-CSFR+ by flow cytometry on Day 0. Starting on the day of adoptive cell transfer (Day 0), mice were randomized to different cytokine treatment groups. In the “daily” group, mice were randomized to receive either vehicle, 130a1 G-CSF (10 μg/dose), or pegylated (PEG) 130a1 G-CSF (10 μg/dose) on the following schedule: every day for 14 days, followed by every second day for 14 days. In the “every three days” group, mice were randomized to receive either 130a1 G-CSF or pegylated 130a1 G-CSF every three days for 9 cycles. In the “weekly” group, mice were randomized to receive either 130a1 G-CSF or pegylated 130a1 G-CSF every seven days for four cycles.

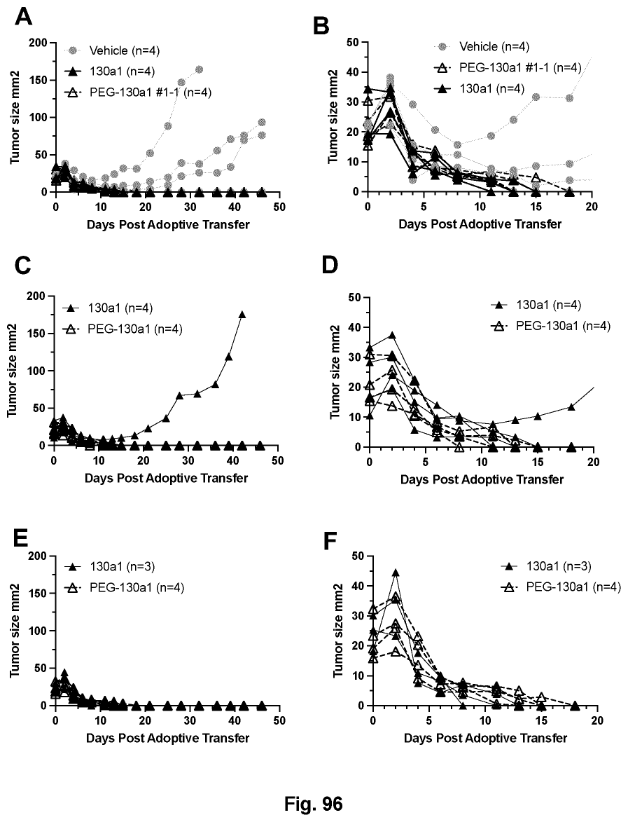

In the vehicle-treated group, 1/4 mice experienced complete regression (CR) of their tumor and remained tumor-free until day 46, when the study was stopped for practical reasons (FIG. 96A, B). In both the “daily” 130a1 G-CSF and PEG-130a1 G-CSF-treated groups, 4/4 mice experienced CR. In the “every three days” 130a1 G-CSF and PEG-130a1 G-CSF-treated groups, 3/4 and 4/4 mice experienced CR, respectively (FIG. 96C,D). In the “weekly” 130a1 G-CSF and PEG-130a1 G-CSF-treated groups, 3/3 and 4/4 mice experienced CR, respectively (FIG. 96E,F).

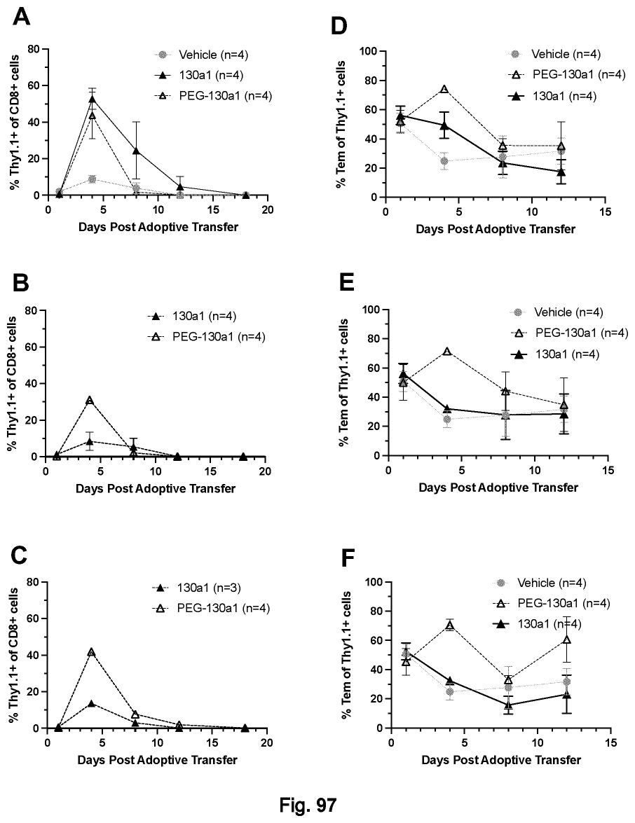

The expansion and persistence of Thy1.1+ OT-I cells were monitored by flow cytometry in serial blood samples collected from mice (FIG. 97A-C). In the “daily” cohort, on Day 4 the Thy1.1+ OT-I cells reached a mean peak level of 8.85%, 52.75% and 43.63% (relative to all CD8⁺ T cells) for the vehicle, 130a1 G-CSF and PEG-130a1 G-CSF treatment groups, respectively (FIG. 97A). In the “every three days” cohort, on Day 4 the Thy1.1+ OT-I cells reached a mean peak level of 8.41% and 31.13% for the 130a1 G-CSF and PEG-130a1 G-CSF treatment groups, respectively (FIG. 97B). In the “weekly” cohort, on Day 4 the Thy1.1+ OT-I cells reached a mean peak level of 13.63% and 41.93% for the 130a1 G-CSF and PEG-130a1 G-CSF treatment groups, respectively (FIG. 97C).

The T effector memory (Tem, CD44+CD62L−) phenotype of Thy1.1+ OT-I cells was also monitored by flow cytometry in serial blood samples (FIG. 97D-F). In the “daily” cohort, on Day 4 the mean percentage of Thy1.1+ OT-I cells exhibiting a Tem phenotype was 24.50%, 49.35% and 74.40% for the vehicle, 130a1 G-CSF and PEG-130a1 G-CSF treatment groups, respectively (FIG. 97D). In the “every three days” cohort, on Day 4 the mean percentage of Thy1.1+ OT-I cells exhibiting a Tem phenotype was 32.03% and 71.53% for the 130a1 G-CSF and PEG-130a1 G-CSF treatment groups, respectively (FIG. 97E). In the “weekly” cohort, on Day 4 the mean percentage of Thy1.1+ OT-I cells exhibiting a Tem phenotype was 32.33% and 70.58% for the 130a1 G-CSF and PEG-130a1 G-CSF treatment groups, respectively (FIG. 97F).

Expression of the Programmed Death-1 (PD-1) molecule was also assessed by flow cytometry in serial blood samples. No significant differences were observed in the percentage of PD-1+ OT-I T cells between the 130a1 G-CSF and PEG-130a1 G-CSF treatment groups at any time point across any of the three dosing schedules (data not shown).

Expression of the T cell immunoglobulin and mucin domain-containing protein 3 (TIM-3) molecule was also assessed by flow cytometry in serial blood samples. No significant differences were observed in the percentage of TIM-3+ OT-I T cells between the 130a1 G-CSF and PEG-130a1 G-CSF treatment groups at any time point across any of the three dosing schedules (data not shown).

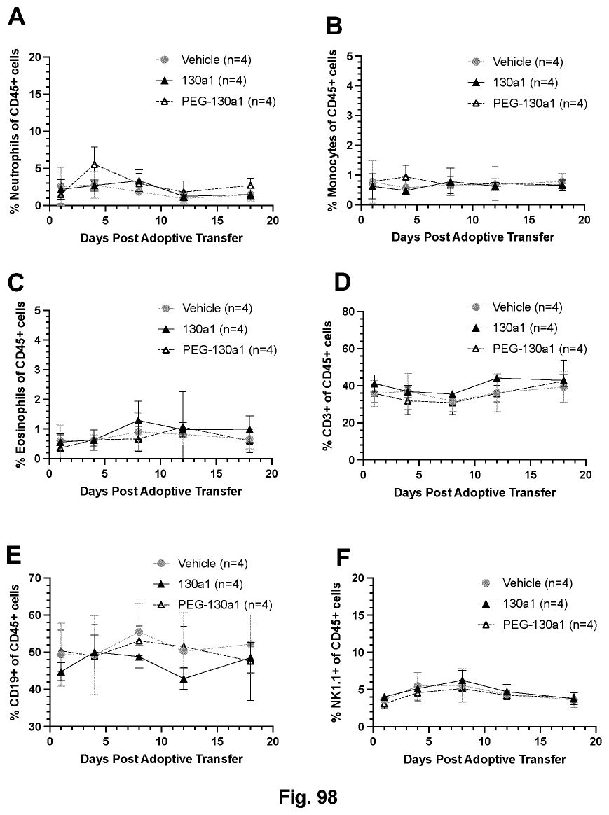

FIG. 98 shows flow cytometry-based assessment of host neutrophils, monocytes, eosinophils, CD3+ T cells, CD19+ B cells and NK1.1+ Natural Killer cells from serial blood samples from the “every three days” dosing cohort. No substantial differences were observed in the percentage of any of these cell types between the vehicle, 130a1 G-CSF or PEG-130a1 G-CSF treatment groups at any time point.

Throughout the study, all mice in all treatment groups maintained a normal body weight and exhibited good general health, despite tumor progression in some.

Thus, PEG-130a1 G-CSF appears to be at least equivalent to 130a1 G-CSF in terms of anti-tumor efficacy in this model. Compared to 130a1 G-CSF, PEG-130a1 G-CSF induced substantially greater OT-I T cell expansion with the “every three days” and weekly dosing schedules. With all three dosing schedules, PEG-130a1 G-CSF induced a larger proportion of OT-I T cells to adopt a Tem phenotype compared to 130a1 G-CSF. There were no overt toxicities associated with treatment with either form of 130a1 G-CSF.

### Example 59: In Vitro Demonstration of a Controlled Paracrine Signaling Strategy that Emulates a Combination of IL-2, IL-12 and IL-18 Signals in Human T Cells

Human CD4 and CD8 T cells from healthy donor PBMCs were transduced with a bi-cistronic vector encoding a mesothelin-specific CAR and G12/2R, or a tri-cistronic vector encoding a mesothelin-specific CAR, G12/2R 134 ECD and human IL-18. T cells were assessed in vitro for expression of the CAR and G12/2R-1 134 ECD; for proliferation, and for IL-18 production.

### Methods

Using methods similar to Example 35, human CD4 and CD8 T cells from healthy donor PBMC were transduced with a tri-cistronic lentiviral vector encoding (in order from 5′ to 3′) a mesothelin-specific CAR, T2A ribosomal skip site, G12/2R-1 134 ECD, T2A ribosomal skip site, and the mature form of human IL-18 (SEQ ID NO: 102). Controls included non-transduced T cells and T cells transduced with a bi-cistronic lentiviral vector encoding a mesothelin-specific CAR, T2A ribosomal skip site, and G12/2R 134 ECD. Non-transduced T cells were expanded for 12 days in IL-2; transduced T cells were expanded in 130a1 G-CSF. After expansion, T cells were subjected to flow cytometry with an antibody specific for G-CSFR ECD (APC-conjugated) and with recombinant Fc-tagged mesothelin protein (FITC-conjugated).

T cell proliferation was assessed by BrdU incorporation assay as per Example 60 with the following cytokines: human IL-2 (Proleukin, 300 IU/ml); human IL-18 (100 ng/ml); human IL-2+ human IL-12 (100 ng/ml); IL-2+IL-12+IL-18; or 130a1 G-CSF (100 ng/ml).

Human IL-18 production was detected by ELISA. T cells were plated at 1×10⁶/ml and cultured in media containing medium alone; human IL-2 (Proleukin, 300 IU/ml)+ human IL-12 (20 ng/ml); or 130a1 G-CSF (100 ng/ml). Supernatants were collected after 48 h and subjected to ELISA (R&D Systems, Human Total IL-18 DuoSet ELISA kit, cat. #DY318-05 used with DuoSet ELISA Ancillary Reagent kit, cat. #DY008) according to the manufacturer's instructions. Absolute IL-18 concentrations were determined using a standard curve provided with the kit.

### Results

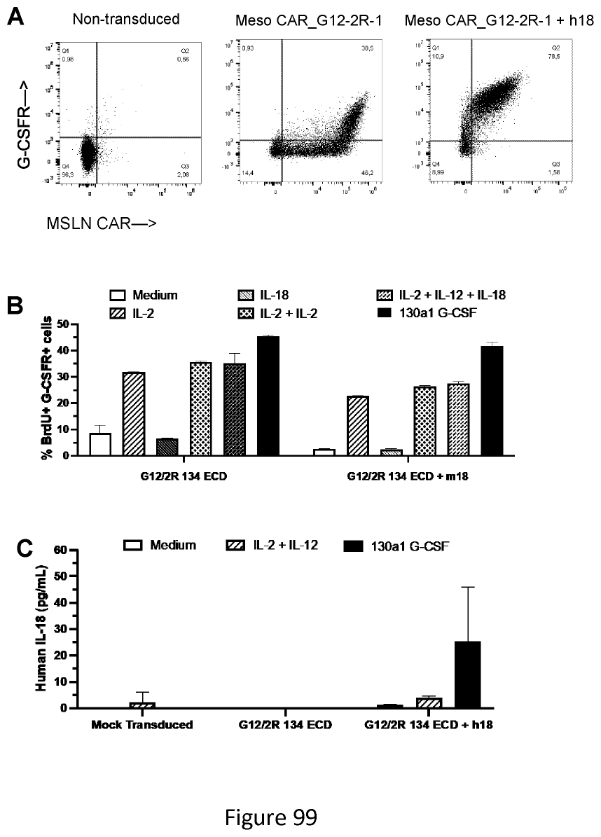

As shown in FIG. 99A, co-expression of the mesothelin CAR and G12/2R 134 ECD was seen in 38.18% of T cells transduced with the bi-cistronic vector and 78.40% of T cells transduced with the tri-cistronic vector. As expected, neither receptor was detected in non-transduced T cells.

As shown in FIG. 99B, T cells transduced with either the bi-cistronic or tri-cistronic vectors proliferated in response to IL-2; IL-2+IL-12; IL-2+IL-12+IL-18; or 130a1 G-CSF; no proliferation was seen in response to medium or IL-18 alone. Similar results were seen for both the CD4+ and CD8+ subsets of T cells (data not shown).

As shown in FIG. 99C, human IL-18 was produced by T cells expressing the tri-cistronic vector and stimulated with 130a1 G-CSF. Negligible levels of IL-18 were detected in the other conditions.

Thus, in human T cells transduced with the vector encoding G12/2R 134 ECD and human IL-18 (along with a mesothelin-specific CAR in this example), T cell proliferation was induced in response to 130a1 G-CSF, as well as various IL-2-containing cytokine combinations. Expression of IL-18 was induced in response to 130a1 G-CSF.

### Example 60: In Vitro Demonstration of an IL-18-Based Controlled Paracrine Signaling System in Murine T Cells

Murine CD4 and CD8 T cells were transduced with a retroviral vector encoding G12/2R 134 ECD or G12/2R 134 ECD plus the mature form of murine IL-18 (m18). Expression of transduced receptors was assessed by flow cytometry. T cells were assessed for proliferation and production of murine IL-18.

### Methods

Using methods similar to Example 36, bulk CD4 and CD8 T cells were isolated from a healthy donor mouse (C57Bl/6J), activated with soluble anti-CD3 and anti-CD28, and expanded for 4 days in the presence of human IL-2 (300 IU/ml). Cells were transduced with a retroviral vector encoding G12/2R-1 134 ECD or G12/2R-1 134 ECD+m18. Mock-transduced T cells underwent the transduction protocol but without retrovirus. Following expansion, cells were washed and re-plated in the following cytokine conditions: medium alone; human IL-2 (Proleukin, 300 IU/ml); murine IL-18 (100 ng/ml); IL-2+mIL-12 (10 ng/ml); IL-2+IL-12+18; or 130a1 G-CSF (100 ng/ml). T cell proliferation was assessed by BrdU incorporation 48 hours later. To assess IL-18 production, transduced T cells were washed and plated at a density of 2×10⁶ cells/ml for 48 hours in medium alone; human IL-2 (300 IU/ml)+murine IL-12 (10 ng/ml); or 130a1 G-CSF (100 ng/ml). Murine IL-18 was detected in cell culture supernatants by ELISA (R&D Systems, Mouse IL-18 ELISA kit, Cat. #7625).

### Results

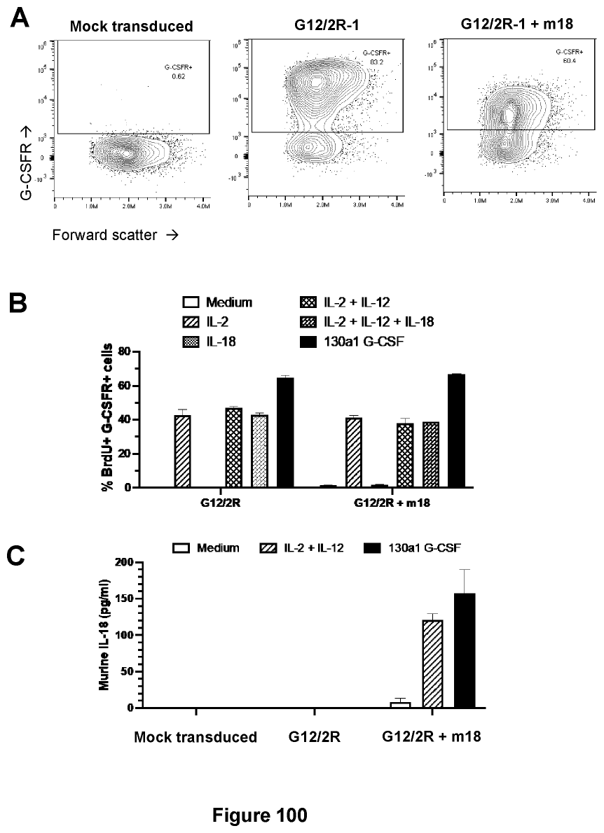

As shown in FIG. 100A, expression of the human G-CSFR ECD was detected on all transduced T cell populations, indicating successful transduction. Expression of G12/2R 134 ECD was lower in the context of the IL-18 CPS compared to on its own; this appears to reflect a technical problem with this retroviral vector, as this issue was not seen in human T cells transduced with the equivalent lentiviral vector (see Example 57).

As shown in FIG. 100B, T cells transduced with G12/2R 134 ECD or G12/2R 134 ECD+m18 proliferated in response to IL-2, IL-2+IL-12, IL-2+IL-12+IL-18, or 130a1 G-CSF.

As shown in FIG. 100C, murine IL-18 was produced by T cells transduced with G12/2R 134 ECD+m18 and stimulated with IL-2+IL-12 or 130a1 G-CSF.

Thus, in murine T cells transduced with G12/2R 134 ECD+m18, stimulation with 130a1 G-CSF induces proliferation and murine IL-18 secretion.

### Example 61: In Vitro Cytokine Secretion Profiles of Murine T Cells Stimulated Through Engineered Receptors and IL-18 CPS

OT-I T cells were transduced with retroviral vectors encoding G2R-2, G2R-3, G12/2R-1, or G12/2R-1+m18 (all with 134 ECD). Expression of transduced receptors was assessed by flow cytometry. Expression of IL-18 and 32 other cytokines was assessed with a multiplexed bead-based assay.

### Methods

Using methods similar to Example 36, OT-I T cells from a healthy donor mouse were transduced with retroviral vectors encoding G2R-2, G2R-3, G12/2R-1 or G12/2R-1+m18 (all with 134 ECD). Five days later, T cells were subjected to flow cytometry with an antibody specific for G-CSFR ECD. To detect secreted cytokines, T cells were washed three times with PBS and cultured in 48-well plates at 2×10⁶ cells/ml in tissue culture medium supplemented with either nothing (media alone, negative control), human IL-2 (Proleukin 300 IU/ml), human IL-2+murine IL-12 (10 ng/ml), or PEG-130a1 G-CSF (100 ng/ml). After 48 hours, culture supernatants were harvested and frozen at −80° C. Supernatants were shipped to Eve Technologies (Calgary, Alberta, Canada) and subjected to a bead-based multiplex cytokine assay (Mouse Cytokine/Chemokine 31-Plex Discovery Assay® Array, MD31) and an IL-18 detection assay (Mouse IL-18 Single Plex Discovery Assay® Array, MDIL18).

### Results

As shown in FIG. 100A, expression of the human G-CSFR ECD was detected on all transduced T cell populations, indicating successful transduction. Expression of G12/2R 134 ECD was lower in the context of the IL-18 CPS compared to on its own; this appears to reflect a technical problem with this retroviral vector, as this issue was not seen in human T cells transduced with the equivalent lentiviral vector (see Example 57).

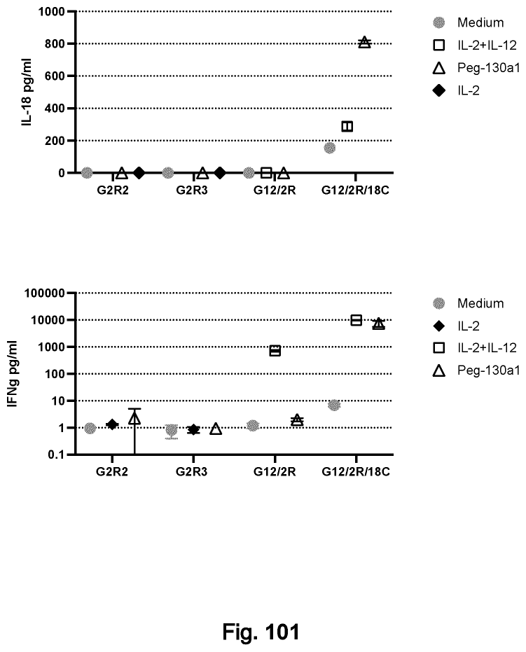

The upper panel of FIG. 101 shows results for murine IL-18 production. Only T cells expressing G12/2R 134 ECD+m18 produced detectable levels of murine IL-18. Expression of murine IL-18 by these cells was increased by stimulation with IL-2+IL-12 or PEG-130a1 G-CSF.

As shown in Table 35, expression of the following cytokines was induced by IL-2 or IL-2+IL-12: GM-CSF, TNF-alpha, IL-10 and VEGF. IL-2+IL-12 additionally induced expression of IFN-gamma (Table 35 and lower panel of FIG. 101). In T cells expressing G12/2R 134 ECD+m18, IL-2+IL-12 additionally induced expression of IP-10 and increased expression of IFN-gamma, TNF-alpha and GM-CSF (Table 35).

In T cells expressing G2R-2 134 ECD, G2R-3 134 ECD or G12/2R-1 134 ECD (without IL-18 CPS), PEG-130a1 G-CSF induced expression of the following cytokines: GM-CSF, TNF-alpha, IL-10, and VEGF. In T cells expressing G12/2R-1 134 ECD+m18, PEG-130a1 G-CSF additionally induced expression of IFN-gamma and IP-10 and increased expression of GM-CSF, TNF-alpha and IL-10 (Table 35).

Thus, in response to PEG-130a1 G-CSF, G2R-2 and G2R-3 induced cytokine secretion patterns resembling those induced by IL-2. In cells expressing G2/12R 134 ECD+m18, stimulation with IL-2+IL-12 or PEG-130a1 G-CSF induced increased production of IL-18, and stimulation with PEG-130a1 G-CSF induced cytokine secretion patterns resembling those induced by IL-2+IL-12.

### Example 62: A Controlled Paracrine Signaling Strategy Emulating a Combination of IL-2, IL-12 and IL-18 Signals Leads to Enhanced Anti-Tumor Activity in a Murine Mammary Carcinoma Model

Using the NOP 23 mammary tumor model, OT-I T cells engineered with a retroviral vector encoding G12/2R-1 134 ECD+m18 were assessed for in vivo expansion, effector phenotype, and anti-tumor activity. For comparison, other groups of mice received OT-I T cells expressing G7R-1, G2R-2 or G12/2R-1 (all with 134 ECD).

### Methods

We used similar methods as described in Example 36. Thy1.1+ OT-I cells were retrovirally transduced to express G7R-1, G2R-2, G12/2R-1 or G12/2R-1+m18 (all with 134 ECD). The antibody panel used for flow cytometry of blood samples was similar to that in Example 59.

### Results

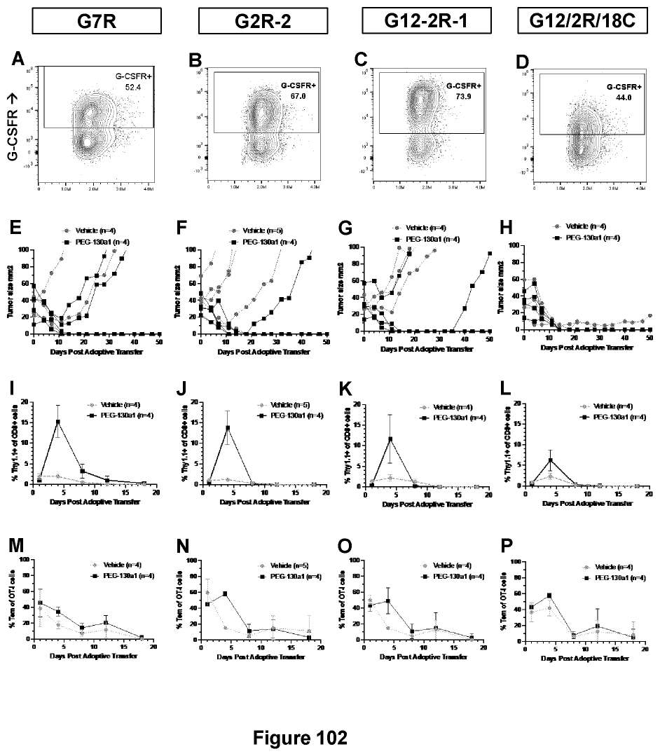

As shown in FIG. 102A-D, on the day of adoptive cell transfer (Day 0), transduced OT-I T cells expressed G7R, G2R-2 or G12/2R-1 (all with 134 ECD), as detected using an antibody against human G-CSFR. Expression of G12/2R-1 134 ECD was lower in the context of the IL-18 CPS compared to on its own; this appears to reflect a technical problem with this retroviral vector, as this issue was not seen in human T cells transduced with the equivalent lentiviral vector (see Example 59).

All mice received an equivalent dose (2.5×10⁶) of transduced Thy1.1+ OT-I cells. After adoptive cell transfer (Day 0), mice were randomized to receive either vehicle or PEG-130a1 G-CSF (10 μg/dose) every seven days for four cycles.

In the G7R-1 cohort, 2/4 mice receiving vehicle alone experienced complete regression (CR) of their tumor compared to 2/4 4 mice in the PEG-130a1 G-CSF treatment group (FIG. 102E). In the G2R-2 cohort, 1/5 mice receiving vehicle alone experienced complete regression (CR) of their tumor compared to 3/4 mice in the PEG-130a1 G-CSF treatment group (FIG. 102F). In the G12/2R-1 cohort, 1/4 mice receiving vehicle alone experienced CR compared to 3/4 mice in the PEG-130a1 G-CSF treatment group, although one of these latter animals experienced tumor recurrence after about 35 days (FIG. 102G). In the G12/2R-1+m18 cohort, 3/4 mice receiving vehicle alone experienced CR compared to 4/4 mice in the PEG-130a1 G-CSF treatment group (FIG. 102H). In all cohorts, mice experiencing durable CR remained tumor-free until day 53, when the study was stopped for practical reasons.

The expansion and persistence of Thy1.1+ OT-I cells were monitored by flow cytometry in serial blood samples collected from mice. In the G7R cohort, on Day 4 the Thy1.1+ OT-I cells reached a mean peak level of 1.99% and 15.31% (relative to all CD8⁺ T cells) for the vehicle and PEG-130a1 G-CSF treatment groups, respectively (FIG. 102I). In the G2R-2 cohort, on Day 4 the Thy1.1+ OT-I cells reached a mean peak level of 1.21% and 13.78% for the vehicle and PEG-130a1 G-CSF treatment groups, respectively (FIG. 102J). In the G12/2R-1 cohort, the Thy1.1+ OT-I cells reached a mean peak level of 2.18% and 11.63% for the vehicle and PEG-130a1 G-CSF treatment groups, respectively (FIG. 102K). In the G12/2R-1+m18 cohort, the Thy1.1+ OT-I cells reached a mean peak level of 2.35% and 6.21% for the vehicle and PEG-130a1 G-CSF treatment groups, respectively (FIG. 102L).

The T effector memory (Tem, CD44+CD62L−) phenotype of Thy1.1+ OT-I cells was also monitored by flow cytometry in serial blood samples. In the G7R cohort, on Day 4 the mean percentage of Thy1.1+ OT-I cells exhibiting a Tem phenotype was 17.75% and 34.65% for the vehicle and PEG-130a1 G-CSF treatment groups, respectively (FIG. 102M). In the G2R-2 cohort, these values were 15.12% and 57.85% for the vehicle and PEG-130a1 G-CSF treatment groups, respectively (FIG. 102N). In the G12/2R-1 cohort, these values were 14.78% and 48.80% for the vehicle and PEG-130a1 G-CSF treatment groups, respectively (FIG. 102O). In the G12/2R/18C cohort, these values were 42.03% and 57.55% for the vehicle and PEG-130a1 G-CSF treatment groups, respectively (FIG. 102P).

Throughout the study, all mice in all treatment groups maintained a normal body weight and exhibited good general health, despite tumor progression in some.

Thus, the extent of OT-I in vivo expansion and effector differentiation in response to pegylated 130a1 G-CSF is influenced by the receptor intracellular domain and CPS cytokine. G12/2R-1+m18 showed the strongest anti-tumor efficacy in this model.

### Example 63: In Vivo Dose-Response Characteristics of PEG-130a1 G-CSF Compared to IL-2 in a Murine Mammary Carcinoma Model

Using the NOP23 mammary tumor model, OT-I T cells expressing G2R-3 134 ECD were assessed for in vivo expansion and effector phenotype in response to different doses of PEG-130a1 G-CSF or human IL-2 (Proleukin). The effects of these cytokines on neutrophil and eosinophil counts in peripheral blood were also assessed.

### Methods

We used similar methods as described in Example 36. Thy1.1+ OT-I cells were retrovirally transduced to express G2R-3 134 ECD. All mice received a dose of 2.5×10⁶ OT-I T cells. On the day of adoptive cell transfer (Day 0), mice were randomized to six cytokine treatment groups: (1) vehicle alone; (2) PEG-130a1 G-CSF 2 μg/dose, four weekly doses; (3) PEG-130a1 G-CSF 0.4 μg/dose, four weekly doses; (4) PEG-130a1 G-CSF 0.08 μg/dose, four weekly doses; (5) PEG-130a1 G-CSF four daily doses of 0.1 μg followed by three weekly doses of 0.4 μg; or (6) human IL-2 (Proleukin) at 30,000 IU/day for 14 days followed by 30,000 IU every two days for 14 days. The antibody panel used for flow cytometry of blood samples was similar to that used in Example 55.

### Results

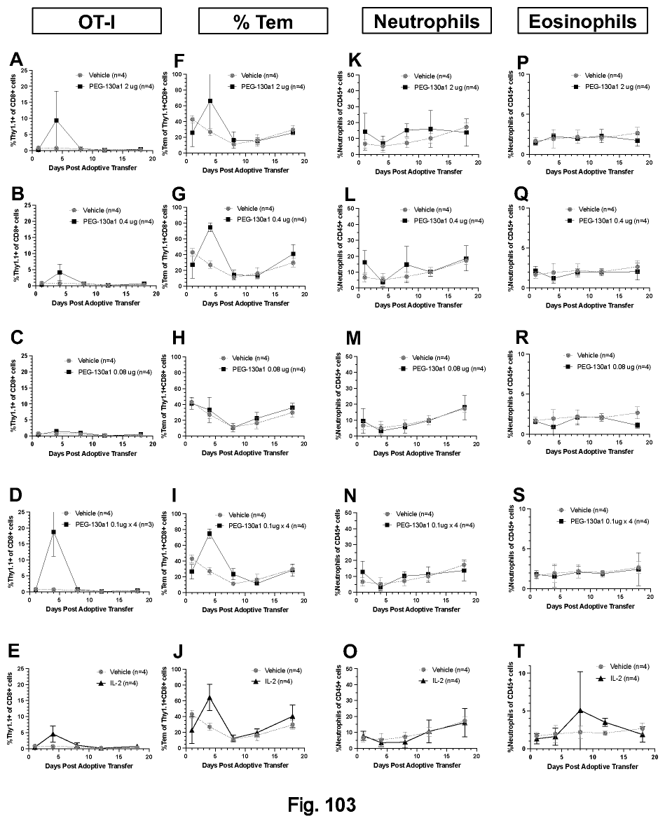

The expansion and persistence of Thy1.1+ OT-I cells were monitored by flow cytometry in serial blood samples collected from mice. FIG. 103A-E shows mean peak percentages of Thy1.1+ OT-I cells (relative to all CD8⁺ T cells) on Day 4 across the different cytokine treatment groups: vehicle alone group=0.70% (shown in each panel); (A) 2 μg PEG-130a1 G-CSF group=9.38%; (B) 0.4 μg PEG-130a1 G-CSF group=4.09%; (C) 0.08 μg PEG-130a1 G-CSF group=1.50%; (D) 0.1 then 0.4 μg PEG-130a1 G-CSF group=18.80%; (E) human IL-2 group=4.56%.

The T effector memory (Tem, CD44+CD62L−) phenotype of Thy1.1+ OT-I cells was also monitored by flow cytometry in serial blood samples. FIG. 103F-J shows the mean percentage of Thy1.1+ OT-I cells exhibiting a Tem phenotype on Day 4: vehicle group=27.05% (shown in each panel); (A) 2 μg PEG-130a1 G-CSF group=66.38%; (B) 0.4 μg PEG-130a1 G-CSF group=74.80%; (C) 0.08 μg PEG-130a1 G-CSF group=33.03%; (D) 0.1 then 0.4 μg PEG-130a1 G-CSF group=74.90%; (E) human IL-2 group=64.30%.

Across time points, all cytokine treatment groups showed similar levels of neutrophils compared to the vehicle group (FIG. 103K-O).

Across time points, all cytokine treatment groups showed similar levels of eosinophils compared to the vehicle group (FIG. 103P-S), with the exception of the IL-2-treated group, which showed an increase in eosinophils on Days 8 and 12 (FIG. 103T).

Throughout the study, all mice in all treatment groups maintained a normal body weight and exhibited good general health with one exception: one mouse in the IL-2-treated group died on Day 19, possibly due to IL-2-associated toxicity.

Thus, the extent of OT-I T cell in vivo expansion and effector differentiation in peripheral blood is influenced by the dose of pegylated 130a1 G-CSF. Pegylated 130a1 G-CSF and IL-2 induced similar OT-I T cell expansion and differentiation profiles in blood. At the assessed doses, neither cytokine had a significant impact on neutrophil levels in blood. IL-2 uniquely induced eosinophilia and may have caused one treatment-related death.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES

Wall E M, Milne K, Martin M L, Watson P H, Theiss P, Nelson B H (2007) Spontaneous mammary tumors differ widely in their inherent sensitivity to adoptively transferred T cells. Cancer Res 67:6442-6450
Yang, T., Martin, M. L., Nielsen, J. S., Milne, K., Wall, E. M., Lin, W., Watson, P. H., & Nelson, B. H. (2009). Mammary tumors with diverse immunological phenotypes show differing sensitivity to adoptively transferred CD8+ T cells lacking the Cbl-b gene. Cancer Immunology, Immunotherapy, 58(11), 1865-1875.

TABLE 12
SEQUENCE OF G-CSFRWT-ICD_{GP130-IL-2Rβ}

| | | Residue | | SEQ |
|---|---|---|---|---|
| | Uniprot | number- | Polypeptide | ID |
| Protein | ID | ing | Sequence | NO. |
| G-CSFR | Q99062 | 1- | MARLGNCSLTWAALIILLLP | 3 |
| (Signal | | 627 | GSLEECGHISVSAPIVHLGD | |
| peptide, | | | PITASCIIKQNCSHLDPEPQ | |
| ECD) | | | ILWRLGAELQPGGRQQRLSD | |
| | | | GTQESIITLPHLNHTQAFLS | |
| | | | CCLNWGNSLQILDQVELRAG | |
| | | | YPPAIPHNLSCLMNLTTSSL | |
| | | | ICQWEPGPETHLPTSFTLKS | |
| | | | FKSRGNCQTQGDSILDCVPK | |
| | | | DGQSHCCIPRKHLLLYQNMG | |
| | | | IWVQAENALGTSMSPQLCLD | |
| | | | PMDVVKLEPPMLRTMDPSPE | |
| | | | AAPPQAGCLQLCWEPWQPGL | |
| | | | HINQKCELRHKPQRGEASWA | |
| | | | LVGPLPLEALQYELCGLLPA | |
| | | | TAYTLQIRCIRWPLPGHWSD | |
| | | | WSPSLELRTTERAPTVRLDT | |
| | | | WWRQRQLDPRTVQLFWKPVP | |
| | | | LEEDSGRIQGYVVSWRPSGQ | |
| | | | AGAILPLCNTTELSCTFHLP | |
| | | | SEAQEVALVAYNSAGTSRPT | |
| | | | PVVFSESRGPALTRLHAMAR | |
| | | | DPHSLWVGWEPPNPWPQGYV | |
| | | | IEWGLGPPSASNSNKTWRME | |
| | | | QNGRATGFLLKENIRPFQLY | |
| | | | EIIVTPLYQDTMGPSQHVYA | |
| | | | YSQEMAPSHAPELHLKHIGK | |
| | | | TWAQLEWVPEPPELGKSPLT | |
| | | | HYTIFWTNAQNQSFSAILNA | |
| | | | SSRGFVLHGLEPASLYHIHL | |
| | | | MAASQAGAINSTVLTLMTLT | |
| | | | PEGSELH | |
| gp130 | P40189 | 628- | AIVVPVCLAFLLTTLLGVLF | 4 |
| (TM, | | 751 | CFNKRDLIKKHIWPNVPDPS | |
| ICD) | | | KSHIAQWSPHTPPRHNFNSK | |
| | | | DQMYSDGNFTDVSVVEIEAN | |
| | | | DKKPFPEDLKSLDLFKKEKI | |
| | | | NTEGHSSGIGGSSCMSSSRP | |
| | | | SIS | |
| IL- | P14784 | 752- | ASLSSNHSLTSCFTNQGYFF | 5 |
| 2RβICD) | | 857 | FHLPDALEIEACQVYFTYDP | |
| | | | YSEEDPDEGVAGAPTGSSPQ | |
| | | | PLQPLSGEDDAYCTFPSRDD | |
| | | | LLLFSPSLLGGPSPPSTAPG | |
| | | | GSGAGEERMPPSLQERVPRD | |
| | | | WDPQPLGPPTPGVPDLVDFQ | |
| | | | PPPELVREGVSFPWSRPPGQ | |
| | | | GEFRALNARLPLNTDAYLSL | |
| | | | QELQGQDPTHLVLREAGEEV | |
| | | | PDAGP | |

TABLE 13
SEQUENCE OF G-CSFRWT-ICD_{IL-2Rβ}

| | Uni- | Residue | | SEQ |
|---|---|---|---|---|
| | prot | number- | Polypeptide | ID |
| Protein | ID | ing | Sequence | NO. |
| G-CSFR | Q99062 | 1- | MARLGNCSLTWAALIILLLP | 6 |
| (Signal | | 627 | GSLEECGHISVSAPIVHLGD | |
| peptide, | | | PITASCIIKQNCSHLDPEPQ | |
| ECD) | | | ILWRLGAELQPGGRQQRLSD | |
| | | | GTQESIITLPHLNHTQAFLS | |
| | | | CCLNWGNSLQILDQVELRAG | |
| | | | YPPAIPHNLSCLMNLTTSSL | |
| | | | ICQWEPGPETHLPTSFTLKS | |
| | | | FKSRGNCQTQGDSILDCVPK | |
| | | | DGQSHCCIPRKHLLLYQNMG | |
| | | | IWVQAENALGTSMSPQLCLD | |
| | | | PMDVVKLEPPMLRTMDPSPE | |
| | | | AAPPQAGCLQLCWEPWQPGL | |
| | | | HINQKCELRHKPQRGEASWA | |
| | | | LVGPLPLEALQYELCGLLPA | |
| | | | TAYTLQIRCIRWPLPGHWSD | |
| | | | WSPSLELRTTERAPTVRLDT | |
| | | | WWRQRQLDPRTVQLFWKPVP | |
| | | | LEEDSGRIQGYVVSWRPSGQ | |
| | | | AGAILPLCNTTELSCTFHLP | |
| | | | SEAQEVALVAYNSAGTSRPT | |
| | | | PVVFSESRGPALTRLHAMAR | |
| | | | DPHSLWVGWEPPNPWPQGYV | |
| | | | IEWGLGPPSASNSNKTWRME | |
| | | | QNGRATGFLLKENIRPFQLY | |
| | | | EIIVTPLYQDTMGPSQHVYA | |
| | | | YSQEMAPSHAPELHLKHIGK | |
| | | | TWAQLEWVPEPPELGKSPLT | |
| | | | HYTIFWTNAQNQSFSAILNA | |
| | | | SSRGFVLHGLEPASLYHIHL | |
| | | | MAASQAGATNSTVLTLMTLT | |
| | | | PEGSELH | |
| IL-2Rβ | P14784 | 628- | AIVVPVCLAFLLTTLLGVLF | 7 |
| (TM and | | 956 | CFNKRDLIKKHIWPNVPDPS | |
| ICD) | | | KSHIAQWSPHTPPRHNFNSK | |
| | | | DQMYSDGNFTDVSVVEIEAN | |
| | | | DKKPFPEDLKSLDLFKKEKI | |
| | | | NTEGHSSGIGGSSCMSSSRP | |
| | | | SISASLSSNHSLTSCFTNQG | |
| | | | YFFFHLPDALEIEACQVYFT | |
| | | | YDPYSEEDPDEGVAGAPTGS | |
| | | | SPQPLQPLSGEDDAYCTFPS | |
| | | | RDDLLLFSPSLLGGPSPPST | |
| | | | APGGSGAGEERMPPSLQERV | |
| | | | PRDWDPQPLGPPTPGVPDLV | |
| | | | DFQPPPELVLREAGEEVPDA | |
| | | | GPREGVSFPWSRPPGQGEFR | |
| | | | ALNARLPLNTDAYLSLQELQ | |
| | | | GQDPTHLV | |

TABLE 14
SEQUENCE OF G-CSFR_{WT-ICDγC}

| | Uni- | Residue | | SEQ |
|---|---|---|---|---|
| | prot | number- | Polypeptide | ID |
| Protein | ID | ing | Sequence | NO. |
| G-CSFR | Q99062 | 1- | MARLGNCSLTWAALIILLLP | 8 |
| (Signal | | 627 | GSLEECGHISVSAPIVHLGD | |
| peptide, | | | PITASCIIKQNCSHLDPEPQ | |
| ECD) | | | ILWRLGAELQPGGRQQRLSD | |
| | | | GTQESIITLPHLNHTQAFLS | |
| | | | CCLNWGNSLQILDQVELRAG | |
| | | | YPPAIPHNLSCLMNLTTSSL | |
| | | | ICQWEPGPETHLPTSFTLKS | |
| | | | FKSRGNCQTQGDSILDCVPK | |
| | | | DGQSHCCIPRKHLLLYQNMG | |
| | | | IWVQAENALGTSMSPQLCLD | |
| | | | PMDVVKLEPPMLRTMDPSPE | |
| | | | AAPPQAGCLQLCWEPWQPGL | |
| | | | HINQKCELRHKPQRGEASWA | |
| | | | LVGPLPLEALQYELCGLLPA | |
| | | | TAYTLQIRCIRWPLPGHWSD | |
| | | | WSPSLELRTTERAPTVRLDT | |
| | | | WWRQRQLDPRTVQLFWKPVP | |
| | | | LEEDSGRIQGYVVSWRPSGQ | |
| | | | AGAILPLCNTTELSCTFHLP | |
| | | | SEAQEVALVAYNSAGTSRPT | |
| | | | PVVFSESRGPALTRLHAMAR | |
| | | | DPHSLWVGWEPPNPWPQGYV | |
| | | | IEWGLGPPSASNSNKTWRME | |
| | | | QNGRATGFLLKENIRPFQLY | |
| | | | EIIVTPLYQDTMGPSQHVYA | |
| | | | YSQEMAPSHAPELHLKHIGK | |
| | | | TWAQLEWVPEPPELGKSPLT | |
| | | | HYTIFWTNAQNQSFSAILNA | |
| | | | SSRGFVLHGLEPASLYHIHL | |
| | | | MAASQAGATNSTVLTLMTLT | |
| | | | PEGSELH | |
| IL-2Rγ | P31785 | 628- | VVISVGSMGLIISLLCVYFW | 9 |
| (TM and | | 735 | LERTMPRIPTLKNLEDLVTE | |
| ICD) | | | YHGNFSAWSGVSKGLAESLQ | |
| | | | PDYSERLCLVSEIPPKGGAL | |
| | | | GEGPGASPCNQHSPYWAPPC | |
| | | | YTLKPET | |

TABLE 15A
CHIMERIC CYTOKINE RECEPTORS

| | | | | | STAT | Other |
|---|---|---|---|---|---|---|
| Name | ECD | TMD | Box1/2 | ICD | site(s) | site(s) |
| G2R-1 | G-CSFR | IL-2Rβ + γc | IL-2Rβ + γc | IL-2Rβ + γc | 5, 5 | Shc |
| G2R-2 | G-CSFR | gp130 | gp130 | IL-2Rβ | 5, 5 | Shc |
| G2R-3 | G-CSFR | G-CSFR | G-CSFR | IL-2Rβ | 5, 5 | Shc |
| G21R-1 | G-CSFR | G-CSFR | G-CSFR | IL-21R | 3 | |
| G21R-2 | G-CSFR | G-CSFR | G-CSFR | IL-21R | 3 | unknown |
| G12R-1 | G-CSFR | G-CSFR | G-CSFR | IL-12Rβ₂ | 4 | |

TABLE 15B
CHIMERIC CYTOKINE RECEPTORS

| | | | | | STAT | Other |
|---|---|---|---|---|---|---|
| Name | ECD | TMD | Box1/2 | ICD | site(s) | site(s) |
| G21/2R-1 | G-CSFR | G-CSFR | G-CSFR | G-CSFR, IL-2Rβ | 3, 5, 5 | Shc |
| G12/2R-1 | G-CSFR | G-CSFR | G-CSFR | STAT4 site from IL- | 4, 5 | Shc |
| | | | | 12Rβ₂ replaces | | |
| | | | | STAT5 site from IL- | | |
| | | | | 2Rβ | | |
| G21/12/ | G-CSFR | G-CSFR | G-CSFR | Contains STAT3 site | 3, 4, 5 | Shc |
| 2R-1 | | | | from G-CSFR ICD; | | |
| | | | | STAT4 site from IL- | | |
| | | | | 12Rβ₂ replaces | | |
| | | | | STAT5 site from IL- | | |
| | | | | 2Rβ | | |
| G27/2R-1 | G-CSFR | gp130 | gp130 | gp130 + IL-2Rβ | 3, 5, 5 | SHP-2, |
| | | | | | | Shc |
| G7R-1 | G-CSFR | G-CSFR | G-CSFR | IL-7Rα | 5 | PI3K |

TABLE 16
BOX 1 AND OTHER BINDING SITES ON CYTOKINE RECEPTOR
ICDS

| | | SEQ | | | | | | | |
|---|---|---|---|---|---|---|---|---|---|
| Native | | ID | | | | | | | |
| ICD | Box 1 (source) | NO: | Shc | SHP-2 | STAT5 | STAT4 | STAT3 | STAT1 | PI3K |
| G-CSFR | LWPSVPDPA | | n/a | n/a | n/a | n/a | Y704 | n/a | n/a |
| | SEQ ID | | | | | | | | |
| | NO. 232) | | | | | | | | |
| | (Q99062) | | | | | | | | |
| gp130 | IWPNVPDPS | | n/a | Y759 | n/a | n/a | Y767 | n/a | n/a |
| | (SEQ ID | | | | | | | | |
| | NO. 233) | | | | | | | | |
| | (P40189) | | | | | | | | |
| IL-2Rβ | LKCNTPDPS | | Y338 | n/a | Y392, | n/a | Y392, | Y392, | n/a |
| | (SEQ ID | | | | Y510 | | Y510 | Y510 | |
| | NO. 234) | | | | | | | | |
| | (P14784) | | | | | | | | |
| IL-21R | KIWAVPSPE | | n/a | n/a | n/a | n/a | Y510 | Y510 | n/a |
| | (Q9HBE5) SEQ | | | | | | | | |
| | ID NO. 235) | | | | | | | | |
| IL- | CSREIPDPA SEQ | | n/a | n/a | Y800 | Y800 | Y800 | n/a | n/a |
| 12Rβ₂ | ID NO. 236) | | | | | | | | |
| | (Q99665) | | | | | | | | |
| IL-7Rα | VWPSLPDHK | | n/a | n/a | Y449 | n/a | n/a | n/a | Y449 |
| | SEQ ID | | | | | | | | |
| | NO. 237) | | | | | | | | |
| | (P16871) | | | | | | | | |

TABLE 17
SIGNAL PEPTIDES

| | | | SEQ |
|---|---|---|---|
| | | | ID |
| | | | NO. |
| | Uniprot | Polypeptide | |
| Domain | ID | Sequence | |
| G-CSFR | Q99062 | MARLGNCSLTW | 11 |
| (Signal | | AALIILLLPGS | |
| peptide) | | LE | |
| GM-CSFR- | P15509 | MLLLVTSLLLC | 12 |
| alpha | | ELPHPAFLLIP | |
| (Signal | | | |
| peptide) | | | |
| | Genbank | Nucleic | |
| | Accession | Acid | |
| Domain | ID | Sequence | |
| G-CSFR | NP_000751 | ATGGCAAGGCTG | 13 |
| (Signal | | GGAAACTGCAGC | |
| peptide, | | CTGACTTGGGCT | |
| native | | GCCCTGATCATC | |
| sequence) | | CTGCTGCTCCCC | |
| | | GGAAGTCTGGAG | |
| GM-CSFR- | NP_034100 | ATGCTGCTGCTA | 14 |
| alpha | | GTGACCTCCCTG | |
| (Signal | | CTGCTCTGTGAG | |
| peptide, | | CTGCCTCACCCG | |
| native | | GCGTTCCTGCTG | |
| sequence) | | ATTCCT | |

TABLE 18
WILD-TYPE G-CSFR EXTRACELLULAR
DOMAINS (ECD):

| | | | SEQ |
|---|---|---|---|
| | | | ID |
| | | | NO. |
| | Uniprot | Polypeptide | |
| Domain | ID | Sequence | |
| G-CSFR (ECD) | Q99062 | ECGHISVSAPIVHLG | 15 |
| | | DPITASCIIKQNCSH | |
| | | LDPEPQILWRLGAEL | |
| | | QPGGRQQRLSDGTQE | |
| | | SIITLPHLNHTQAFL | |
| | | SCCLNWGNSLQILDQ | |
| | | VELRAGYPPAIPHNL | |
| | | SCLMNLTTSSLICQW | |
| | | EPGPETHLPTSFTLK | |
| | | SFKSRGNCQTQGDSI | |
| | | LDCVPKDGQSHCCIP | |
| | | RKHLLLYQNMGIWVQ | |
| | | AENALGTSMSPQLCL | |
| | | DPMDVVKLEPPMLRT | |
| | | MDPSPEAAPPQAGCL | |
| | | QLCWEPWQPGLHINO | |
| | | KCELRHKPQRGEASW | |
| | | ALVGPLPLEALQYEL | |
| | | CGLLPATAYTLQIRC | |
| | | IRWPLPGHWSDWSPS | |
| | | LELRTTERAPTVRLD | |
| | | TWWRQRQLDPRTVQL | |
| | | FWKPVPLEEDSGRIQ | |
| | | GYVVSWRPSGQAGAI | |
| | | LPLCNTTELSCTFHL | |
| | | PSEAQEVALVAYNSA | |
| | | GTSRPTPVVFSESRG | |
| | | PALTRLHAMARDPHS | |
| | | LWVGWEPPNPWPQGY | |
| | | VIEWGLGPPSASNSN | |
| | | KTWRMEQNGRATGFL | |
| | | LKENIRPFQLYEIIV | |
| | | TPLYQDTMGPSQHVY | |
| | | AYSQEMAPSHAPELH | |
| | | LKHIGKTWAQLEWVP | |
| | | EPPELGKSPLTHYTI | |
| | | FWTNAQNQSFSAILN | |
| | | ASSRGFVLHGLEPAS | |
| | | LYHIHLMAASQAGAT | |
| | | NSTVLTLMTLTPEGS | |
| | | ELH | |
| | Genbank | | |
| | Accession | Nucleic Acid | |
| Domain | ID | Sequence | |
| G-CSFR (ECD, | NP_000751 | GAGTGCGGGCACATC | 16 |
| native | | AGTGTCTCAGCCCCC | |
| sequence) | | ATCGTCCACCTGGGG | |
| | | GATCCCATCACAGCC | |
| | | TCCTGCATCATCAAG | |
| | | CAGAACTGCAGCCAT | |
| | | CTGGACCCGGAGCCA | |
| | | CAGATTCTGTGGAGA | |
| | | CTGGGAGCAGAGCTT | |
| | | CAGCCCGGGGGCAGG | |
| | | CAGCAGCGTCTGTCT | |
| | | GATGGGACCCAGGAA | |
| | | TCTATCATCACCCTG | |
| | | CCCCACCTCAACCAC | |
| | | ACTCAGGCCTTTCTC | |
| | | TCCTGCTGCCTGAAC | |
| | | TGGGGCAACAGCCTG | |
| | | CAGATCCTGGACCAG | |
| | | GTTGAGCTGCGCGCA | |
| | | GGCTACCCTCCAGCC | |
| | | ATACCCCACAACCTC | |
| | | TCCTGCCTCATGAAC | |
| | | CTCACAACCAGCAGC | |
| | | CTCATCTGCCAGTGG | |
| | | GAGCCAGGACCTGAG | |
| | | ACCCACCTACCCACC | |
| | | AGCTTCACTCTGAAG | |
| | | AGTTTCAAGAGCCGG | |
| | | GGCAACTGTCAGACC | |
| | | CAAGGGGACTCCATC | |
| | | CTGGACTGCGTGCCC | |
| | | AAGGACGGGCAGAGC | |
| | | CACTGCTGCATCCCA | |
| | | CGCAAACACCTGCTG | |
| | | TTGTACCAGAATATG | |
| | | GGCATCTGGGTGCAG | |
| | | GCAGAGAATGCGCTG | |
| | | GGGACCAGCATGTCC | |
| | | CCACAACTGTGTCTT | |
| | | GATCCCATGGATGTT | |
| | | GTGAAACTGGAGCCC | |
| | | CCCATGCTGCGGACC | |
| | | ATGGACCCCAGCCCT | |
| | | GAAGCGGCCCCTCCC | |
| | | CAGGCAGGCTGCCTA | |
| | | CAGCTGTGCTGGGAG | |
| | | CCATGGCAGCCAGGC | |
| | | CTGCACATAAATCAG | |
| | | AAGTGTGAGCTGCGC | |
| | | CACAAGCCGCAGCGT | |
| | | GGAGAAGCCAGCTGG | |
| | | GCACTGGTGGGCCCC | |
| | | CTCCCCTTGGAGGCC | |
| | | CTTCAGTATGAGCTC | |
| | | TGCGGGCTCCTCCCA | |
| | | GCCACGGCCTACACC | |
| | | CTGCAGATACGCTGC | |
| | | ATCCGCTGGCCCCTG | |
| | | CCTGGCCACTGGAGC | |
| | | GACTGGAGCCCCAGC | |
| | | CTGGAGCTGAGAACT | |
| | | ACCGAACGGGCCCCC | |
| | | ACTGTCAGACTGGAC | |
| | | ACATGGTGGCGGCAG | |
| | | AGGCAGCTGGACCCC | |
| | | AGGACAGTGCAGCTG | |
| | | TTCTGGAAGCCAGTG | |
| | | CCCCTGGAGGAAGAC | |
| | | AGCGGACGGATCCAA | |
| | | GGTTATGTGGTTTCT | |
| | | TGGAGACCCTCAGGC | |
| | | CAGGCTGGGGCCATC | |
| | | CTGCCCCTCTGCAAC | |
| | | ACCACAGAGCTCAGC | |
| | | TGCACCTTCCACCTG | |
| | | CCTTCAGAAGCCCAG | |
| | | GAGGTGGCCCTTGTG | |
| | | GCCTATAACTCAGCC | |
| | | GGGACCTCTCGTCCC | |
| | | ACTCCGGTGGTCTTC | |
| | | TCAGAAAGCAGAGGC | |
| | | CCAGCTCTGACCAGA | |
| | | CTCCATGCCATGGCC | |
| | | CGAGACCCTCACAGC | |
| | | CTCTGGGTAGGCTGG | |
| | | GAGCCCCCCAATCCA | |
| | | TGGCCTCAGGGCTAT | |
| | | GTGATTGAGTGGGGC | |
| | | CTGGGCCCCCCCAGC | |
| | | GCGAGCAATAGCAAC | |
| | | AAGACCTGGAGGATG | |
| | | GAACAGAATGGGAGA | |
| | | GCCACGGGGTTTCTG | |
| | | CTGAAGGAGAACATC | |
| | | AGGCCCTTTCAGCTC | |
| | | TATGAGATCATCGTG | |
| | | ACTCCCTTGTACCAG | |
| | | GACACCATGGGACCC | |
| | | TCCCAGCATGTCTAT | |
| | | GCCTACTCTCAAGAA | |
| | | ATGGCTCCCTCCCAT | |
| | | GCCCCAGAGCTGCAT | |
| | | CTAAAGCACATTGGC | |
| | | AAGACCTGGGCACAG | |
| | | CTGGAGTGGGTGCCT | |
| | | GAGCCCCCTGAGCTG | |
| | | GGGAAGAGCCCCCTT | |
| | | ACCCACTACACCATC | |
| | | TTCTGGACCAACGCT | |
| | | CAGAACCAGTCCTTC | |
| | | TCCGCCATCCTGAAT | |
| | | GCCTCCTCCCGTGGC | |
| | | TTTGTCCTCCATGGC | |
| | | CTGGAGCCCGCCAGT | |
| | | CTGTATCACATCCAC | |
| | | CTCATGGCTGCCAGC | |
| | | CAGGCTGGGGCCACC | |
| | | AACAGTACAGTCCTC | |
| | | A | |
| | | CCCTGATGACCTTGA | |
| | | CCCCAGAGGGGTCGG | |
| | | AGCTACAC | |
| G-CSFR | | GAGTGCGGGCACATC | 17 |
| (ECD, 4 | | AGTGTCTCAGCCCCC | |
| codons | | ATCGTCCACCTGGGG | |
| altered) | | GATCCCATCACAGCC | |
| | | TCCTGCATCATCAAG | |
| | | CAGAACTGCAGCCAT | |
| | | CTGGACCCGGAGCCA | |
| | | CAGATTCTGTGGAGA | |
| | | CTGGGAGCAGAGCTT | |
| | | CAGCCCGGGGGCAGG | |
| | | CAGCAGCGTCTGTCT | |
| | | GATGGGACCCAGGAA | |
| | | TCTATCATCACCCTG | |
| | | CCCCACCTCAACCAC | |
| | | ACTCAGGCCTTTCTC | |
| | | TCCTGCTGCCTGAAC | |
| | | TGGGGCAACAGCCTG | |
| | | CAGATCCTGGACCAG | |
| | | GTTGAGCTGCGCGCA | |
| | | GGCTACCCTCCAGCC | |
| | | ATACCCCACAACCTC | |
| | | TCCTGCCTCATGAAC | |
| | | CTCACAACCAGCAGC | |
| | | CTCATCTGCCAGTGG | |
| | | GAGCCAGGACCTGAG | |
| | | ACCCACCTACCCACC | |
| | | AGCTTCACTCTGAAG | |
| | | AGTTTCAAGAGCCGG | |
| | | GGCAACTGTCAGACC | |
| | | CAAGGGGACTCCATC | |
| | | CTGGACTGCGTGCCC | |
| | | AAGGACGGGCAGAGC | |
| | | CACTGCTGCATCCCA | |
| | | CGCAAACACCTGCTG | |
| | | TTGTACCAGAATATG | |
| | | GGCATCTGGGTGCAG | |
| | | GCAGAGAATGCGCTG | |
| | | GGGACCAGCATGTCC | |
| | | CCACAACTGTGTCTT | |
| | | GATCCCATGGATGTT | |
| | | GTGAAACTGGAGCCC | |
| | | CCCATGCTGCGGACC | |
| | | ATGGACCCCAGCCCT | |
| | | GAAGCGGCCCCTCCC | |
| | | CAGGCAGGCTGCCTA | |
| | | CAGCTGTGCTGGGAG | |
| | | CCATGGCAGCCAGGC | |
| | | CTGCACATAAATCAG | |
| | | AAGTGTGAGCTGCGC | |
| | | CACAAGCCGCAGCGT | |
| | | GGAGAAGCCAGCTGG | |
| | | GCACTGGTGGGCCCC | |
| | | CTCCCCTTGGAGGCC | |
| | | CTTCAGTATGAGCTC | |
| | | TGCGGGCTCCTCCCA | |
| | | GCCACGGCCTACACC | |
| | | CTGCAGATACGCTGC | |
| | | ATCCGCTGGCCCCTG | |
| | | CCTGGCCACTGGAGC | |
| | | GACTGGAGCCCCAGC | |
| | | CTGGAGCTGAGAACT | |
| | | ACCGAACGGGCCCCC | |
| | | ACTGTCAGACTGGAC | |
| | | ACATGGTGGCGGCAG | |
| | | AGGCAGCTGGACCCC | |
| | | AGGACAGTGCAGCTG | |
| | | TTCTGGAAGCCAGTG | |
| | | CCCCTGGAGGAAGAC | |
| | | AGCGGACGCATCCAA | |
| | | GGTTATGTGGTTTCT | |
| | | TGGAGACCCTCAGGC | |
| | | CAGGCTGGGGCCATC | |
| | | CTGCCCCTCTGCAAC | |
| | | ACCACAGAGCTCAGC | |
| | | TGCACCTTCCACCTG | |
| | | CCTTCAGAAGCCCAG | |
| | | GAGGTGGCCCTTGTG | |
| | | GCCTATAACTCAGCC | |
| | | GGGACCTCTCGCCCC | |
| | | ACCCCGGTGGTCTTC | |
| | | TCAGAAAGCAGAGGC | |
| | | CCAGCTCTGACCAGA | |
| | | CTCCATGCCATGGCC | |
| | | CGAGACCCTCACAGC | |
| | | CTCTGGGTAGGCTGG | |
| | | GAGCCCCCCAATCCA | |
| | | TGGCCTCAGGGCTAT | |
| | | GTGATTGAGTGGGGC | |
| | | CTGGGCCCCCCCAGC | |
| | | GCGAGCAATAGCAAC | |
| | | AAGACCTGGAGGATG | |
| | | GAACAGAATGGGAGA | |
| | | GCCACGGGGTTTCTG | |
| | | CTGAAGGAGAACATC | |
| | | AGGCCCTTTCAGCTC | |
| | | TATGAGATCATCGTG | |
| | | ACTCCCTTGTACCAG | |
| | | GACACCATGGGACCC | |
| | | TCCCAGCATGTCTAT | |
| | | GCCTACTCTCAAGAA | |
| | | ATGGCTCCCTCCCAT | |
| | | GCCCCAGAGCTGCAT | |
| | | CTAAAGCACATTGGC | |
| | | AAGACCTGGGCACAG | |
| | | CTGGAGTGGGTGCCT | |
| | | GAGCCCCCTGAGCTG | |
| | | GGGAAGAGCCCCCTT | |
| | | ACCCACTACACCATC | |
| | | TTCTGGACCAACGCT | |
| | | CAGAACCAGTCCTTC | |
| | | TCCGCCATCCTGAAT | |
| | | GCATCCTCCCGTGGC | |
| | | TTTGTCCTCCATGGC | |
| | | CTGGAGCCCGCCAGT | |
| | | CTGTATCACATCCAC | |
| | | CTCATGGCTGCCAGC | |
| | | CAGGCTGGGGCCACC | |
| | | AACAGTACAGTCCTC | |
| | | ACCCTGATGACCTTG | |
| | | ACCCCAGAGGGGTCG | |
| | | GAGCTACAC | |

TABLE 19
TRANSMEMBRANE DOMAINS (TM)

| | | | SEQ |
|---|---|---|---|
| | | | ID |
| | | | NO. |
| | | Polypeptide | |
| Domain | Uniprot ID | Sequence | |
| G-CSFR (TM) | Q99062 | IILGLFGLLL | 18 |
| | | LLTCLCGTAW | |
| | | LCC | |
| gp130 (TM) | P40189 | AIVVPVCLAF | 19 |
| | | LLTTLLGVLF | |
| | | CF | |
| IL-2Rb (TM) | P14784 | IPWLGHLLVG | 20 |
| | | LSGAFGFIIL | |
| | | VYLLI | |
| IL-2RG (TM) | P31785 | VVISVGSMGL | 21 |
| | | IISLLCVYFW | |
| | | L | |
| | Genbank | Nucleic Acid | |
| Domain | Accession ID | Sequence | |
| G-CSFR (TM) | NP_000751 | ATCATCCTGG | 22 |
| | | GCCTGTTCGG | |
| | | CCTCCTGCTG | |
| | | TTGCTCAC | |
| | | CTGCCTCTGT | |
| | | GGAACTGCCT | |
| | | GGCTCTGTTG | |
| | | C | |
| gp130 (TM) | NM_002184 | GCCATAGTCG | 23 |
| | | TGCCTGTTTG | |
| | | CTTAGCATTC | |
| | | CTATTGAC | |
| | | AACTCTTCTG | |
| | | GGAGTGCTGT | |
| | | TCTGCTTT | |
| IL-2Rb (TM) | NM_000878 | ATTCCGTGGC | 24 |
| | | TCGGCCACCT | |
| | | CCTCGTGGGT | |
| | | CTCAGCGG | |
| | | GGCTTTTGGC | |
| | | TTCATCATCT | |
| | | TAGTGTACTT | |
| | | GCTGATC | |
| IL-2RG (TM) | NP_000197 | GTGGTTATCT | 25 |
| | | CTGTTGGCTC | |
| | | CATGGGATTG | |
| | | ATTATCAG | |
| | | CCTTCTCTGT | |
| | | GTGTATTTCT | |
| | | GGCTG | |

TABLE 20
INTRACELLULAR DOMAINS (ICD)

| | | | SEQ |
|---|---|---|---|
| | Uniprot | Polypeptide | ID |
| Domain | ID | Sequence | NO. |
| IL-2Rb | P14784 | NCRNTGPWLKKVLKCNTPDPSKFFS | 26 |
| (ICD, G2R-1) | | QLSSEHGGDVQKWLSSPFPSSSFSP | |
| | | GGLAPEISPLEVLERDKVTQLLLQQ | |
| | | DKVPEPASLSSNHSLTSCFTNQGYF | |
| | | FFHLPDALEIEACQVYFTYDPYSEE | |
| | | DPDEGVAGAPTGSSPQPLQPLSGED | |
| | | DAYCTFPSRDDLLLFSPSLLGGPSP | |
| | | PSTAPGGSGAGEERMPPSLQERVPR | |
| | | DWDPQPLGPPTPGVPDLVDFQPPPE | |
| | | LVLREAGEEVPDAGPREGVSFPWSR | |
| | | PPGQGEFRALNARLPLNTDAYLSLQ | |
| | | ELQGQDPTHLV* | |
| IL-2RG | P31785 | ERTMPRIPTLKNLEDLVTEYHGNFS | 27 |
| (ICD, G2R-1) | | AWSGVSKGLAESLQPDYSERLCLVS | |
| | | EIPPKGGALGEGPGASPCNQHSPYW | |
| | | APPCYTLKPET* | |
| gp130 | P40189 | NKRDLIKKHIWPNVPDPSKSHIAQW | 28 |
| (ICD, G2R-2) | | SPHTPPRHNFNSKDQMYSDGNFTDV | |
| | | SVVEIEANDKKPFPEDLKSLDLFKK | |
| | | EKINTEGHSSGIGGSSCMSSSRPSI | |
| | | S | |
| IL-2Rb | P14784 | ASLSSNHSLTSCFTNQGYFFFHLPD | 29 |
| (ICD, G2R-2) | | ALEIEACQVYFTYDPYSEEDPDEGV | |
| | | AGAPTGSSPQPLQPLSGEDDAYCTF | |
| | | PSRDDLLLFSPSLLGGPSPPSTAPG | |
| | | GSGAGEERMPPSLQERVPRDWDPQP | |
| | | LGPPTPGVPDLVDFQPPPELVLREA | |
| | | GEEVPDAGPREGVSFPWSRPPGQGE | |
| | | FRALNARLPLNTDAYLSLQELQGQD | |
| | | PTHLV* | |
| G-CSFR | Q99062 | SPNRKNPLWPSVPDPAHSSLGSWVP | 30 |
| (ICD, G2R-3) | | TIMEEDAFQLPGLGTPPITKLTVLE | |
| | | EDEKKPVPWESH | |
| IL-2Rb | P14784 | SSNHSLTSCFTNQGYFFFHLPDALE | 31 |
| (ICD, G2R-3) | | IEACQVYFTYDPYSEEDPDEGVAGA | |
| | | PTGSSPQPLQPLSGEDDAYCTFPSR | |
| | | DDLLLFSPSLLGGPSPPSTAPGGSG | |
| | | AGEERMPPSLQERVPRDWDPQPLGP | |
| | | PTPGVPDLVDFQPPPELVLREAGEE | |
| | | VPDAGPREGVSFPWSRPPGQGEFRA | |
| | | LNARLPLNTDAYLSLQELQGQDPTH | |
| | | LV* | |
| G-CSFR | Q99062 | SPNRKNPLWPSVPDPAHSSLGSWVP | 32 |
| (ICD, G12R-1) | | TIMEEDAFQLPGLGTPPITKLTVLE | |
| | | EDEKKPVPWESHNSSET | |
| IL-12Rb2 | Q99665 | AGDLPTHDGYLPSNIDDLPSHEAPL | 33 |
| (ICD, G12R-1) | | ADSLEELEPQHISLSVFPSSSLHPL | |
| | | TFSCGDKLTLDQLKMRCDSLML* | |
| G-CSFR | Q99062 | SPNRKNPLWPSVPDPAHSSLGSWVP | 34 |
| (ICD, G21R-1) | | TIMEEDAFQLPGLGTPPITKLTVLE | |
| | | EDEKKPVPWESHNSSET | |
| IL-21R | Q9HBE5 | SPGDEGPPRSYLRQWVVIPPPLSSP | 35 |
| (ICD, G21R-1) | | GPQAS* | |
| G-CSFR | Q99062 | SPNRKNPLWPSVPDPAHSSLGSWVP | 36 |
| (ICD, G21R-2) | | TIMEEDAFQLPGLGTPPITKLTVLE | |
| | | EDEKKPVPWESHN | |
| IL-21R | Q9HBE5 | QNSGGSAYSEERDRPYGLVSIDTVT | 37 |
| (ICD, G21R-2) | | VLDAEGPCTWPCSCEDDGYPALDLD | |
| | | AGLEPSPGLEDPLLDAGTTVLSCGC | |
| | | VSAGSPGLGGPLGSLLDRLKPPLAD | |
| | | GEDWAGGLPWGGRSPGGVSESEAGS | |
| | | PLAGLDMDTFDSGFVGSDCSSPVEC | |
| | | DFTSPGDEGPPRSYLRQWVVIPPPL | |
| | | SSPGPQAS* | |
| G-CSFR | Q99062 | SPNRKNPLWPSVPDPAHSSLGSWVP | 38 |
| (ICD, G21/2R-1) | | TIMEEDAFQLPGLGTPPITKLTVLE | |
| | | EDEKKPVPWESHNSSETCGLPTLVQ | |
| | | TYVLQGDPRAVSTQPQSQ | |
| IL-2Rb | P14784 | SSNHSLTSCFTNQGYFFFHLPDALE | 39 |
| (ICD, G21/2R-1) | | IEACQVYFTYDPYSEEDPDEGVAGA | |
| | | PTGSSPQPLQPLSGEDDAYCTFPSR | |
| | | DDLLLFSPSLLGGPSPPSTAPGGSG | |
| | | AGEERMPPSLQERVPRDWDPQPLGP | |
| | | PTPGVPDLVDFQPPPELVLREAGEE | |
| | | VPDAGPREGVSFPWSRPPGQGEFRA | |
| | | LNARLPLNTDAYLSLQELQGQDPTH | |
| | | LV* | |
| G-CSFR | Q99062 | SPNRKNPLWPSVPDPAHSSLGSWVP | 40 |
| (ICD, G12/2R-1) | | TIMEEDAFQLPGLGTPPITKLTVLE | |
| | | EDEKKPVPWESH | |
| IL-2Rb | P14784 | SSNHSLTSCFTNQGYFFFHLPDALE | 41 |
| (ICD, G12/2R-1) | | IEACQVYFTYDPYSEEDP | |
| IL-12Rb2 | Q99665 | AGDLPTHDGYLPSNIDDLPS | 42 |
| (ICD, G12/2R-1) | | | |
| IL-2Rb | P14784 | GGPSPPSTAPGGSGAGEERMPPSLQ | 43 |
| (ICD, G12/2R-1) | | ERVPRDWDPQPLGPPTPGVPDLVDF | |
| | | QPPPELVLREAGEEVPDAGPREGVS | |
| | | FPWSRPPGQGEFRALNARLPLNTDA | |
| | | YLSLQELQGQDPTHLV* | |
| G-CSFR | Q99062 | SPNRKNPLWPSVPDPAHSSLGSWVP | 44 |
| (ICD, G21/12/2R- | | TIMEEDAFQLPGLGTPPITKLTVLE | |
| 1) | | EDEKKPVPWESHNSSETCGLPTLVQ | |
| | | TYVLQGDPRAVSTQPQSQ | |
| IL-2Rb (ICD, | P14784 | SSNHSLTSCFTNQGYFFFHLPDALE | 45 |
| G21/12/2R-1) | | IEACQVYFTYDPYSEEDP | |
| IL-12Rb2 (ICD, | Q99665 | AGDLPTHDGYLPSNIDDLPS | 46 |
| G21/12/2R-1) | | | |
| IL-2Rb (ICD, | P14784 | GGPSPPSTAPGGSGAGEERMPPSLQ | 47 |
| G21/12/2R-1) | | ERVPRDWDPQPLGPPTPGVPDLVDF | |
| | | QPPPELVLREAGEEVPDAGPREGVS | |
| | | FPWSRPPGQGEFRALNARLPLNTDA | |
| | | YLSLQELQGQDPTHLV* | |
| gp130 | P40189 | NKRDLIKKHIWPNVPDPSKSHIAQW | 48 |
| (ICD, G27/2R-1) | | SPHTPPRHNFNSKDQMYSDGNFTDV | |
| | | SVVEIEANDKKPFPEDLKSLDLFKK | |
| | | EKINTEGHSSGIGGSSCMSSSRPSI | |
| | | SSSDENESSQNTSSTVQYSTVVHSG | |
| | | YRHQVPSVQVFSR | |
| IL-2Rb | P14784 | ASLSSNHSLTSCFTNQGYFFFHLPD | 49 |
| (ICD, G27/2R-1) | | ALEIEACQVYFTYDPYSEEDPDEGV | |
| | | AGAPTGSSPQPLQPLSGEDDAYCTF | |
| | | PSRDDLLLFSPSLLGGPSPPSTAPG | |
| | | GSGAGEERMPPSLQERVPRDWDPQP | |
| | | LGPPTPGVPDLVDFQPPPELVLREA | |
| | | GEEVPDAGPREGVSFPWSRPPGQGE | |
| | | FRALNARLPLNTDAYLSLQELQGQD | |
| | | PTHLV* | |
| G-CSFR | Q99062 | SPNRKNPLWPSVPDPAHSSLGSWVP | 50 |
| (ICD, G7R-1) | | TIMEEDAFQLPGLGTPPITKLTVLE | |
| | | EDEKKPVPWESH | |
| IL-7Ra | P16871 | SGKNGPHVYQDLLLSLGTTNSTLPP | 51 |
| (ICD, G7R-1) | | PFSLQSGILTLNPVAQGQPILTSLG | |
| | | SNQEEAYVTMSSFYQNQ* | |
| G-CSFR | Q99062 | SPNRKNPLWPSVPDPAHSSLGSWVP | 52 |
| (ICD, G21/7R-1) | | TIMEEDAFQLPGLGTPPITKLTVLE | |
| | | EDEKKPVPWESHNSSETCGLPTLVQ | |
| | | TYVLQGDPRAVSTQPQSQ | |
| IL7R | P16871 | SSSRSLDCRESGKNGPHVYQDLLLS | 53 |
| (ICD, G21/7R-1) | | LGTTNSTLPPPFSLQSGILTLNPVA | |
| | | QGQPILTSLGSNQEEAYVTMSSFYQ | |
| | | NQ* | |
| | Genbank | | |
| | Accession | | |
| Domain | ID | Nucleic Acid Sequence | |
| IL-2RB | NM_000878 | AACTGCAGGAACACCGGGCCATGGC | 54 |
| (ICD, G2R-1) | | TGAAGAAGGTCCTGAAGTGTAACAC | |
| | | CCCAGACCCCTCGAAGTTCTTTTCC | |
| | | CAGCTGAGCTCAGAGCATGGAGGAG | |
| | | ACGTCCAGAAGTGGCTCTCTTCGCC | |
| | | CTTCCCCTCATCGTCCTTCAGCCCT | |
| | | GGCGGCCTGGCACCTGAGATCTCGC | |
| | | CACTAGAAGTGCTGGAGAGGGACAA | |
| | | GGTGACGCAGCTGCTCCTGCAGCAG | |
| | | GACAAGGTGCCTGAGCCCGCATCCT | |
| | | TAAGCAGCAACCACTCGCTGACCAG | |
| | | CTGCTTCACCAACCAGGGTTACTTC | |
| | | TTCTTCCACCTCCCGGATGCCTTGG | |
| | | AGATAGAGGCCTGCCAGGTGTACTT | |
| | | TACTTACGACCCCTACTCAGAGGAA | |
| | | GACCCTGATGAGGGTGTGGCCGGGG | |
| | | CACCCACAGGGTCTTCCCCCCAACC | |
| | | CCTGCAGCCTCTGTCAGGGGAGGAC | |
| | | GACGCCTACTGCACCTTCCCCTCCA | |
| | | GGGATGACCTGCTGCTCTTCTCCCC | |
| | | CAGTCTCCTCGGTGGCCCCAGCCCC | |
| | | CCAAGCACTGCCCCTGGGGGCAGTG | |
| | | GGGCCGGTGAAGAGAGGATGCCCCC | |
| | | TTCTTTGCAAGAAAGAGTCCCCAGA | |
| | | GACTGGGACCCCCAGCCCCTGGGGC | |
| | | CTCCCACCCCAGGAGTCCCAGACCT | |
| | | GGTGGATTTTCAGCCACCCCCTGAG | |
| | | CTGGTGCTGCGAGAGGCTGGGGAGG | |
| | | AGGTCCCTGACGCTGGCCCCAGGGA | |
| | | GGGAGTCAGTTTCCCCTGGTCCAGG | |
| | | CCTCCTGGGCAGGGGGAGTTCAGGG | |
| | | CCCTTAATGCTCGCCTGCCCCTGAA | |
| | | CACTGATGCCTACTTGTCCCTCCAA | |
| | | GAACTCCAGGGTCAGGACCCAACTC | |
| | | ACTTGGTGTAG | |
| IL-2RG | NP_000197 | GAACGGACGATGCCCCGAATTCCCA | 55 |
| (ICD, G2R-1) | | CCCTGAAGAACCTAGAGGATCTTGT | |
| | | TACTGAATACCACGGGAACTTTTCG | |
| | | GCCTGGAGTGGTGTGTCTAAGGGAC | |
| | | TGGCTGAGAGTCTGCAGCCAGACTA | |
| | | CAGTGAACGACTCTGCCTCGTCAGT | |
| | | GAGATTCCCCCAAAAGGAGGGGCCC | |
| | | TTGGGGAGGGGCCTGGGGCCTCCCC | |
| | | ATGCAACCAGCATAGCCCCTACTGG | |
| | | GCCCCCCCATGTTACACCCTAAAGC | |
| | | CTGAAACCTGA | |
| gp130 | NM_002184 | AATAAGCGAGACCTAATTAAAAAAC | 56 |
| (ICD, G2R-2) | | ACATCTGGCCTAATGTTCCAGATCC | |
| | | TTCAAAGAGTCATATTGCCCAGTGG | |
| | | TCACCTCACACTCCTCCAAGGCACA | |
| | | ATTTTAATTCAAAAGATCAAATGTA | |
| | | TTCAGATGGCAATTTCACTGATGTA | |
| | | AGTGTTGTGGAAATAGAAGCAAATG | |
| | | ACAAAAAGCCTTTTCCAGAAGATCT | |
| | | GAAATCATTGGACCTGTTCAAAAAG | |
| | | GAAAAAATTAATACTGAAGGACACA | |
| | | GCAGTGGTATTGGGGGGTCTTCATG | |
| | | TATGTCATCTTCTAGGCCAAGCATT | |
| | | TCT | |
| IL-2Rb | NM_000878 | GCATCCTTAAGCAGCAACCACTCGC | 57 |
| (ICD, G2R-2) | | TGACCAGCTGCTTCACCAACCAGGG | |
| | | TTACTTCTTCTTCCACCTCCCGGAT | |
| | | GCCTTGGAGATAGAGGCCTGCCAGG | |
| | | TGTACTTTACTTACGACCCCTACTC | |
| | | AGAGGAAGACCCTGATGAGGGTGTG | |
| | | GCCGGGGCACCCACAGGGTCTTCCC | |
| | | CCCAACCCCTGCAGCCTCTGTCAGG | |
| | | GGAGGACGACGCCTACTGCACCTTC | |
| | | CCCTCCAGGGATGACCTGCTGCTCT | |
| | | TCTCCCCCAGTCTCCTCGGTGGCCC | |
| | | CAGCCCCCCAAGCACTGCCCCTGGG | |
| | | GGCAGTGGGGCCGGTGAAGAGAGGA | |
| | | TGCCCCCTTCTTTGCAAGAAAGAGT | |
| | | CCCCAGAGACTGGGACCCCCAGCCC | |
| | | CTGGGGCCTCCCACCCCAGGAGTCC | |
| | | CAGACCTGGTGGATTTTCAGCCACC | |
| | | CCCTGAGCTGGTGCTGCGAGAGGCT | |
| | | GGGGAGGAGGTCCCTGACGCTGGCC | |
| | | CCAGGGAGGGAGTCAGTTTCCCCTG | |
| | | GTCCAGGCCTCCTGGGCAGGGGGAG | |
| | | TTCAGGGCCCTTAATGCTCGCCTGC | |
| | | CCCTGAACACTGATGCCTACTTGTC | |
| | | CCTCCAAGAACTCCAGGGTCAGGAC | |
| | | CCAACTCACTTGGTGTAG | |
| G-CSFR | NP_000751 | AGCCCCAACAGGAAGAATCCCCTCT | 58 |
| (ICD, G2R-3) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCAT | |
| IL-2Rb | NM_000878 | AGCAGCAACCACTCGCTGACCAGCT | 59 |
| (ICD, G2R-3) | | GCTTCACCAACCAGGGTTACTTCTT | |
| | | CTTCCACCTCCCGGATGCCTTGGAG | |
| | | ATAGAGGCCTGCCAGGTGTACTTTA | |
| | | CTTACGACCCCTACTCAGAGGAAGA | |
| | | CCCTGATGAGGGTGTGGCCGGGGCA | |
| | | CCCACAGGGTCTTCCCCCCAACCCC | |
| | | TGCAGCCTCTGTCAGGGGAGGACGA | |
| | | CGCCTACTGCACCTTCCCCTCCAGG | |
| | | GATGACCTGCTGCTCTTCTCCCCCA | |
| | | GTCTCCTCGGTGGCCCCAGCCCCCC | |
| | | AAGCACTGCCCCTGGGGGCAGTGGG | |
| | | GCCGGTGAAGAGAGGATGCCCCCTT | |
| | | CTTTGCAAGAAAGAGTCCCCAGAGA | |
| | | CTGGGACCCCCAGCCCCTGGGGCCT | |
| | | CCCACCCCAGGAGTCCCAGACCTGG | |
| | | TGGATTTTCAGCCACCCCCTGAGCT | |
| | | GGTGCTGCGAGAGGCTGGGGAGGAG | |
| | | GTCCCTGACGCTGGCCCCAGGGAGG | |
| | | GAGTCAGTTTCCCCTGGTCCAGGCC | |
| | | TCCTGGGCAGGGGGAGTTCAGGGCC | |
| | | CTTAATGCTCGCCTGCCCCTGAACA | |
| | | CTGATGCCTACTTGTCCCTCCAAGA | |
| | | ACTCCAGGGTCAGGACCCAACTCAC | |
| | | TTGGTGTAG | |
| G-CSFR | NP_000751 | AGCCCCAACAGGAAGAATCCCCTCT | 60 |
| (ICD, G12R-1) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCATAACAGCTCAGAGAC | |
| | | C | |
| IL-12Rb2 | NP_001550 | GCAGGTGACCTTCCCACCCATGATG | 61 |
| (ICD, G12R-1) | | GCTACTTACCCTCCAACATAGATGA | |
| | | CCTCCCCTCACATGAGGCACCTCTC | |
| | | GCTGACTCTCTGGAAGAACTGGAGC | |
| | | CTCAGCACATCTCCCTTTCTGTTTT | |
| | | CCCCTCAAGTTCTCTTCACCCACTC | |
| | | ACCTTCTCCTGTGGTGATAAGCTGA | |
| | | CTCTGGATCAGTTAAAGATGAGGTG | |
| | | TGACTCCCTCATGCTCTGA | |
| G-CSFR | NP_000751 | AGCCCCAACAGGAAGAATCCCCTCT | 62 |
| (ICD, G21R-1) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCATAACAGCTCAGAGAC | |
| | | C | |
| IL-21R | NP_068570 | AGCCCTGGGGACGAAGGACCCCCCC | 63 |
| (ICD, G21R-1) | (1codon | GGAGCTACCTCCGCCAGTGGGTGGT | |
| | altered) | CATTCCTCCGCCACTTTCGAGCCCT | |
| | | GGACCCCAGGCCAGCTAA | |
| G-CSFR | NP_000751 | AGCCCCAACAGGAAGAATCCCCTCT | 64 |
| (ICD, G21R-2) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCATAAC | |
| IL-21R | NP_068570 | CAGAACTCGGGGGGCTCAGCTTACA | 65 |
| (ICD, G21R-2) | (1codon | GTGAGGAGAGGGATCGGCCATACGG | |
| | altered) | CCTGGTGTCCATTGACACAGTGACT | |
| | | GTGCTAGATGCAGAGGGGCCATGCA | |
| | | CCTGGCCCTGCAGCTGTGAGGATGA | |
| | | CGGCTACCCAGCCCTGGACCTGGAT | |
| | | GCTGGCCTGGAGCCCAGCCCAGGCC | |
| | | TAGAGGACCCACTCTTGGATGCAGG | |
| | | GACCACAGTCCTGTCCTGTGGCTGT | |
| | | GTCTCAGCTGGCAGCCCTGGGCTAG | |
| | | GAGGGCCCCTGGGAAGCCTCCTGGA | |
| | | CAGACTAAAGCCACCCCTTGCAGAT | |
| | | GGGGAGGACTGGGCTGGGGGACTGC | |
| | | CCTGGGGTGGCCGGTCACCTGGAGG | |
| | | GGTCTCAGAGAGTGAGGCGGGCTCA | |
| | | CCCCTGGCCGGCCTGGATATGGACA | |
| | | CGTTTGACAGTGGCTTTGTGGGCTC | |
| | | TGACTGCAGCAGCCCTGTGGAGTGT | |
| | | GACTTCACCAGCCCTGGGGACGAAG | |
| | | GACCCCCCCGGAGCTACCTCCGCCA | |
| | | GTGGGTGGTCATTCCTCCGCCACTT | |
| | | TCGAGCCCTGGACCCCAGGCCAGC | |
| G-CSFR | NP_000751 | AGCCCCAACAGGAAGAATCCCCTCT | 66 |
| (ICD, G21/2R-1) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCATAACAGCTCAGAGAC | |
| | | CTGTGGCCTCCCCACTCTGGTCCAG | |
| | | ACCTATGTGCTCCAGGGGGACCCAA | |
| | | GAGCAGTTTCCACCCAGCCCCAATC | |
| | | CCAG | |
| IL-2Rb | NM_000878 | AGCAGCAACCACTCGCTGACCAGCT | 67 |
| (ICD, G21/2R-1) | | GCTTCACCAACCAGGGTTACTTCTT | |
| | | CTTCCACCTCCCGGATGCCTTGGAG | |
| | | ATAGAGGCCTGCCAGGTGTACTTTA | |
| | | CTTACGACCCCTACTCAGAGGAAGA | |
| | | CCCTGATGAGGGTGTGGCCGGGGCA | |
| | | CCCACAGGGTCTTCCCCCCAACCCC | |
| | | TGCAGCCTCTGTCAGGGGAGGACGA | |
| | | CGCCTACTGCACCTTCCCCTCCAGG | |
| | | GATGACCTGCTGCTCTTCTCCCCCA | |
| | | GTCTCCTCGGTGGCCCCAGCCCCCC | |
| | | AAGCACTGCCCCTGGGGGCAGTGGG | |
| | | GCCGGTGAAGAGAGGATGCCCCCTT | |
| | | CTTTGCAAGAAAGAGTCCCCAGAGA | |
| | | CTGGGACCCCCAGCCCCTGGGGCCT | |
| | | CCCACCCCAGGAGTCCCAGACCTGG | |
| | | TGGATTTTCAGCCACCCCCTGAGCT | |
| | | GGTGCTGCGAGAGGCTGGGGAGGAG | |
| | | GTCCCTGACGCTGGCCCCAGGGAGG | |
| | | GAGTCAGTTTCCCCTGGTCCAGGCC | |
| | | TCCTGGGCAGGGGGAGTTCAGGGCC | |
| | | CTTAATGCTCGCCTGCCCCTGAACA | |
| | | CTGATGCCTACTTGTCCCTCCAAGA | |
| | | ACTCCAGGGTCAGGACCCAACTCAC | |
| | | TTGGTGTAG | |
| G-CSFR | NP_000751 | AGCCCCAACAGGAAGAATCCCCTCT | 68 |
| (ICD, G12/2R-1) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCAT | |
| IL-2Rb | NM_000878 | AGCAGCAACCACTCGCTGACCAGCT | 69 |
| (ICD, G12/2R-1) | | GCTTCACCAACCAGGGTTACTTCTT | |
| | | CTTCCACCTCCCGGATGCCTTGGAG | |
| | | ATAGAGGCCTGCCAGGTGTACTTTA | |
| | | CTTACGACCCCTACTCAGAGGAAGA | |
| | | CCCT | |
| IL-12Rb2 | NP_001550 | GCAGGTGACCTTCCCACCCATGATG | 70 |
| (ICD, G12/2R-1) | | GCTACTTACCCTCCAACATAGATGA | |
| | | CCTCCCCTCA | |
| IL-2Rb | NM_000878 | GGTGGCCCCAGCCCCCCAAGCACTG | 71 |
| (ICD, G12/2R-1) | | CCCCTGGGGGCAGTGGGGCCGGTGA | |
| | | AGAGAGGATGCCCCCTTCTTTGCAA | |
| | | GAAAGAGTCCCCAGAGACTGGGACC | |
| | | CCCAGCCCCTGGGGCCTCCCACCCC | |
| | | AGGAGTCCCAGACCTGGTGGATTTT | |
| | | CAGCCACCCCCTGAGCTGGTGCTGC | |
| | | GAGAGGCTGGGGAGGAGGTCCCTGA | |
| | | CGCTGGCCCCAGGGAGGGAGTCAGT | |
| | | TTCCCCTGGTCCAGGCCTCCTGGGC | |
| | | AGGGGGAGTTCAGGGCCCTTAATGC | |
| | | TCGCCTGCCCCTGAACACTGATGCC | |
| | | TACTTGTCCCTCCAAGAACTCCAGG | |
| | | GTCAGGACCCAACTCACTTGGTGTA | |
| | | G | |
| G-CSFR | NP_000751 | AGCCCCAACAGGAAGAATCCCCTCT | 72 |
| (ICD, G21/12/2R- | | GGCCAAGTGTCCCAGACCCAGCTCA | |
| 1) | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCATAACAGCTCAGAGAC | |
| | | CTGTGGCCTCCCCACTCTGGTCCAG | |
| | | ACCTATGTGCTCCAGGGGGACCCAA | |
| | | GAGCAGTTTCCACCCAGCCCCAATC | |
| | | CCAG | |
| IL-2Rb | NM_000878 | AGCAGCAACCACTCGCTGACCAGCT | 73 |
| (ICD, G21/12/2R- | | GCTTCACCAACCAGGGTTACTTCTT | |
| 1) | | CTTCCACCTCCCGGATGCCTTGGAG | |
| | | ATAGAGGCCTGCCAGGTGTACTTTA | |
| | | CTTACGACCCCTACTCAGAGGAAGA | |
| | | CCCT | |
| IL-12Rb2 (ICD, | NP_001550 | GCAGGTGACCTTCCCACCCATGATG | 74 |
| G21/12/2R-1) | | GCTACTTACCCTCCAACATAGATGA | |
| | | CCTCCCCTCA | |
| IL-2Rb | NM_000878 | GGTGGCCCCAGCCCCCCAAGCACTG | 75 |
| (ICD, G21/12/2R- | | CCCCTGGGGGCAGTGGGGCCGGTGA | |
| 1) | | AGAGAGGATGCCCCCTTCTTTGCAA | |
| | | GAAAGAGTCCCCAGAGACTGGGACC | |
| | | CCCAGCCCCTGGGGCCTCCCACCCC | |
| | | AGGAGTCCCAGACCTGGTGGATTTT | |
| | | CAGCCACCCCCTGAGCTGGTGCTGC | |
| | | GAGAGGCTGGGGAGGAGGTCCCTGA | |
| | | CGCTGGCCCCAGGGAGGGAGTCAGT | |
| | | TTCCCCTGGTCCAGGCCTCCTGGGC | |
| | | AGGGGGAGTTCAGGGCCCTTAATGC | |
| | | TCGCCTGCCCCTGAACACTGATGCC | |
| | | TACTTGTCCCTCCAAGAACTCCAGG | |
| | | GTCAGGACCCAACTCACTTGGTGTA | |
| | | G | |
| gp130 | NM_002184 | AATAAGCGAGACCTAATTAAAAAAC | 76 |
| (ICD, G27/2R-1) | (3) codons | ACATCTGGCCTAATGTTCCAGATCC | |
| | altered) | TTCAAAGAGTCATATTGCCCAGTGG | |
| | | TCACCTCACACTCCTCCAAGGCACA | |
| | | ATTTCAATTCAAAGGATCAAATGTA | |
| | | TTCAGATGGCAATTTCACTGATGTA | |
| | | AGTGTTGTGGAAATAGAAGCAAATG | |
| | | ACAAAAAGCCTTTTCCAGAAGATCT | |
| | | GAAATCATTGGACCTGTTCAAAAAG | |
| | | GAAAAAATTAATACTGAAGGACACA | |
| | | GCAGTGGTATTGGGGGGTCTTCATG | |
| | | TATGTCATCTTCTAGGCCAAGCATT | |
| | | TCTAGCAGTGATGAAAATGAATCTT | |
| | | CACAAAACACTTCGAGCACTGTCCA | |
| | | GTATTCTACCGTGGTACACAGTGGC | |
| | | TACAGACACCAAGTTCCGTCAGTCC | |
| | | AAGTCTTCTCAAGA | |
| IL-2Rb | NM_000878 | GCATCCTTAAGCAGCAACCACTCGC | 77 |
| (ICD, G27/2R-1) | (2 codons | TGACCAGCTGCTTCACCAACCAGGG | |
| | altered) | TTACTTCTTCTTCCACCTCCCGGAT | |
| | | GCCTTGGAGATAGAGGCCTGCCAGG | |
| | | TGTACTTTACTTACGACCCCTACTC | |
| | | AGAGGAAGACCCTGATGAGGGTGTG | |
| | | GCCGGGGCACCCACAGGGTCTTCCC | |
| | | CCCAACCCCTGCAGCCTCTGTCAGG | |
| | | GGAGGACGACGCCTACTGCACCTTC | |
| | | CCCTCCAGGGATGACCTGCTGCTCT | |
| | | TCTCCCCCAGTCTCCTCGGTGGCCC | |
| | | CAGCCCCCCAAGCACTGCCCCTGGG | |
| | | GGCAGTGGGGCCGGTGAAGAGAGGA | |
| | | TGCCCCCTTCTTTGCAAGAAAGAGT | |
| | | CCCCAGAGACTGGGACCCCCAGCCC | |
| | | CTGGGGCCTCCCACCCCAGGAGTCC | |
| | | CAGACCTGGTGGATTTTCAGCCACC | |
| | | CCCTGAGCTGGTGCTGCGAGAGGCT | |
| | | GGGGAGGAGGTCCCTGACGCTGGCC | |
| | | CCAGGGAGGGAGTCAGTTTCCCCTG | |
| | | GTCCAGGCCTCCTGGGCAGGGGGAG | |
| | | TTCAGGGCCCTTAATGCTCGCCTGC | |
| | | CCCTGAACACTGATGCCTACTTGTC | |
| | | CCTCCAAGAACTCCAGGGTCAGGAC | |
| | | CCAACTCACTTGGTGTAG | |
| G-CSFR | NP_000751 | AGCCCCAACAGGAAGAATCCCCTCT | 78 |
| (ICD, G7R-1) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCAT | |
| IL-7Ra | NP_002176 | AGTGGCAAGAATGGGCCTCATGTGT | 79 |
| (ICD, G7R-1) | | ACCAGGACCTCCTGCTTAGCCTTGG | |
| | | GACTACAAACAGCACGCTGCCCCCT | |
| | | CCATTTTCTCTCCAATCTGGAATCC | |
| | | TGACATTGAACCCAGTTGCTCAGGG | |
| | | TCAGCCCATTCTTACTTCCCTGGGA | |
| | | TCAAATCAAGAAGAAGCATATGTCA | |
| | | CCATGTCCAGCTTCTACCAAAACCA | |
| | | GTGA | |
| G-CSFR | NP_000751 | AGCCCCAACAGGAAGAATCCCCTCT | 80 |
| (ICD, G21/7R-1) | | GGCCAAGTGTCCCAGACCCAGCTCA | |
| | | CAGCAGCCTGGGCTCCTGGGTGCCC | |
| | | ACAATCATGGAGGAGGATGCCTTCC | |
| | | AGCTGCCCGGCCTTGGCACGCCACC | |
| | | CATCACCAAGCTCACAGTGCTGGAG | |
| | | GAGGATGAAAAGAAGCCGGTGCCCT | |
| | | GGGAGTCCCATAACAGCTCAGAGAC | |
| | | CTGTGGCCTCCCCACTCTGGTCCAG | |
| | | ACCTATGTGCTCCAGGGGGACCCAA | |
| | | GAGCAGTTTCCACCCAGCCCCAATC | |
| | | CCAG | |
| IL7R | NP_002176 | TCCTCTTCCAGGTCCCTAGACTGCA | 81 |
| (ICD, G21/7R-1) | | GGGAGAGTGGCAAGAATGGGCCTCA | |
| | | TGTGTACCAGGACCTCCTGCTTAGC | |
| | | CTTGGGACTACAAACAGCACGCTGC | |
| | | CCCCTCCATTTTCTCTCCAATCTGG | |
| | | AATCCTGACATTGAACCCAGTTGCT | |
| | | CAGGGTCAGCCCATTCTTACTTCCC | |
| | | TGGGATCAAATCAAGAAGAAGCATA | |
| | | TGTCACCATGTCCAGCTTCTACCAA | |
| | | AACCAGTGA | |

TABLE 21
PROTEIN SEQUENCE OF WT AND VARIANT FORMS OF
RECOMBINANT HUMAN G-CSF CYTOKINEAND VARIATIONS OF 130A1 SEQ
ID NO 83-1 THROUGH 83-5) DESIGNED TO IMPROVE STABILITY.

| | | Residue | | SEQ ID |
|---|---|---|---|---|
| Protein | Uniprot ID | numbering | Polypeptide Sequence | NO. |
| WT G-CSF | P09919 | *0-174 | MTPLGPASSLPQSFLLKCLEQVRKIQGDGAALQEKL | 82 |
| | | (*0 refers to | CATYKLCHPEELVLLGHSLGIPWAPLSSCPSQALQLA | |
| | | an M residue | GCLSQLHSGLFLYQGLLQALEGISPELGPTLDTLQLD | |
| | | not present in | VADFATTIWQQMEELGMAPALQPTQGAMPAFASAF | |
| | | native G- | QRRAGGVLVASHLQSFLEVSYRVLRHLAQP | |
| | | CSF) | | |
| 130a1 | Derived | *0-174 | MTPLGPASSLPQSFLLKCLEQVRKIQGDGAALQEKL | 83 |
| variant with | from | | CATYKLCHPERLVLLGHSLGIPWAPLSSCPSQALQL | |
| mutations | P09919 | | AGCLSQLHSGLFLYQGLLQALEGISPELGPTLDTLQK | |
| underlined | | | DVARFATTIWQQMKELGMAPALQPTQGAMPAFAS | |
| | | | AFQRRAGGVLVASHLQSFLEVSYRVLRHLAQP | |
| 307 | Derived | *0-174 | MTPLGPASSLPQEFLLDCLKQVRKIQGDGAALQEKL | 84 |
| variant with | from | | CATYKLCHPERLVLLGHSLGIPWAPLSSCPSQALQL | |
| mutations | P09919 | | AGCLSQLHSGLFLYQGLLQALEGISPELGPTLDTLQL | |
| underlined | | | DVADFATTIWQQMEELGMAPALQPTQGAMPAFASA | |
| | | | FORRAGGVLVASHLQSFLEVSYRVLRHLAQP | |

TABLE 22
Protein sequence of WT and variant forms of human G-CSF receptor
extracellular and transmembrane domains and signal peptide

| | | Domain & | | |
|---|---|---|---|---|
| | | residue | | SEQ ID |
| Protein | Uniprot ID | numbering | Polypeptide Sequence | NO. |
| WT G-CSFR | Q99062 | ECD: 2-308 | ECGHISVSAPIVHLGDPITASCIIKQNCSHLDPEPQILWRLGAELQP | 85 |
| | | | GGRQQRLSDGTQESIITLPHLNHTQAFLSCSLNWGNSLQILDQVELR | |
| | | | AGYPPAIPHNLSCLMNLTTSSLICQWEPGPETHLPTSFTLKSFKSR | |
| | | | GNCQTQGDSILDCVPKDGQSHCSIPRKHLLLYQNMGIWVQAENA | |
| | | | LGTSMSPQLCLDPMDVVKLEPPMLRTMDPSPEAAPPQAGCLQLS | |
| | | | WEPWQPGLHINQKCELRHKPQRGEASWALVGPLPLEALQYELCG | |
| | | | LLPATAYTLQIRCIRWPLPGHWSDWSPSLELRTTE | |
| 134 G-CSFR | Derived | ECD: 2-308 | ECGHISVSAPIVHLGDPITASCIIKQNCSHLDPEPQILWELGAELQP | 86 |
| variant | from | | GGRQQRLSDGTQESIITLPHLNHTQAFLSCSLNWGNSLQILDQVELR | |
| (mutations | Q99062 | | AGYPPAIPHNLSCLMNLTTSSLICQWEPGPETHLPTSFTLKSFKSE | |
| underlined) | | | GNCQTQGDSILDCVPKDGQSHCSIPDKHLLLYQNMGIWVQAENA | |
| | | | LGTSMSPQLCLDPMDVVKLEPPMLRTMDPSPEAAPPQAGCLQLS | |
| | | | WEPWQPGLHINQKCELRHKPQRGEASWALVGPLPLEALQYELCG | |
| | | | LLPATAYTLQIRCIRWPLPGHWSDWSPSLELRTTE | |
| 307 G-CSFR | Derived | ECD: 2-308 | ECGHISVSAPIVHLGDPITASCIIKQNCSHLDPEPQILWELGAELQP | 87 |
| variant | from | | GGRQQRLSDGTQESIITLPHLNHTQAFLSCSLNWGNSLQILDQVELR | |
| (mutations | Q99062 | | AGYPPAIPHNLSCLMNLTTSSLICQWEPGPETHLPTSFTLKSFKSR | |
| underlined) | | | GNCQTQGDSILDCVPKDGQSHCSIPRKHLLLYQNMGIWVQAENA | |
| | | | LGTSMSPQLCLKPMKVVKLEPPMLRTMDPSPEAAPPQAGCLQLS | |
| | | | WEPWQPGLHINQKCELRHKPQRGEASWALVGPLPLEALQYELCG | |
| | | | LLPATAYTLQIRCIEWPLPGHWSDWSPSLELRTTE | |
| WT G-CSFR | Q99062 | TMD: 309-331 | IILGLFGLLLLLTCLCGTAWLCC | 88 |
| WT G-CSFR | Q99062 | Signal peptide | MARLGNCSLTWAALIILLLPGSLE | 89 |

TABLE 23
INTRACELLULAR DOMAINS (ICDS)

| Chimeric | | Uniprot | | SEQ ID |
|---|---|---|---|---|
| receptor | Protein (Domain) | ID | Polypeptide Sequence | NO. |
| G2R-3 | G-CSFR: | Q99062 | SPNRKNPLWPSVPDPAHSSLGSWVPTIMEE | 90 |
| | Box 1 underlined | | DAFQLPGLGTPPITKLTVLEEDEKKPVPWE | |
| | Box 2 bold underlined | | SH | |
| | IL-2Rβ: | P14784 | SSNHSLTSCFTNQGYFFFHLPDALEIEACQ | 91 |
| | Shc & STAT5 binding sites | | VYFTYDPYSEEDPDEGVAGAPTGSSPQPLQ | |
| | bold underlined | | PLSGEDDAYCTFPSRDDLLLFSPSLLGGPS | |
| | *stop codon | | PPSTAPGGSGAGEERMPPSLQERVPRDWDP | |
| | | | QPLGPPTPGVPDLVDFQPPPELVLREAGEE | |
| | | | VPDAGPREGVSFPWSRPPGQGEFRALNARL | |
| | | | PLNTDAYLSLQELQGQDPTHLV* | |
| G4R | G-CSFR: | Q99062 | SPNRKNPLWPSVPDPAHSSLGSWVPTIMEE | 90 |
| | Box 1 underlined | | DAFQLPGLGTPPITKLTVLEEDEKKPVPWE | |
| | Box 2 bold underlined | | SH | |
| | IL4Rα: | P24394 | DEDPHKAAKEMPFQGSGKSAWCPVEISKTV | 92 |
| | IRS-1 & 2 and | | LWPESISVVRCVELFEAPVECEEEEEVEEE | |
| | STAT6 binding sites bold | | KGSFCASPESSRDDFQEGREGIVARLTESL | |
| | underlined | | FLDLLGEENGGFCQQDMGESCLLPPSGSTS | |
| | *stop codon | | AHMPWDEFPSAGPKEAPPWGKEQPLHLEPS | |
| | | | PPASPTQSPDNLTCTETPLVIAGNPAYRSF | |
| | | | SNSLSQSPCPRELGPDPLLARHLEEVEPEM | |
| | | | PCVPQLSEPTTVPQPEPETWEQILRRNVLQ | |
| | | | HGAAAAPVSAPTSGYQEFVHAVEQGGTQAS | |
| | | | AVVGLGPPGEAGYKAFSSLLASSAVSPEKC | |
| | | | GFGASSGEEGYKPFQDLIPGCPGDPAPVPV | |
| | | | PLFTFGLDREPPRSPQSSHLPSSSPEHLGL | |
| | | | EPGEKVEDMPKPPLPQEQATDPLVDSLGSG | |
| | | | IVYSALTCHLCGHLKQCHGQEDGGQTPVMA | |
| | | | SPCCGCCCGDRSSPPTTPLRAPDPSPGGVP | |
| | | | LEASLCPASLAPSGISEKSKSSSSFHPAPG | |
| | | | NAQSSSQTPKIVNFVSVGPTYMRVS* | |
| G6R | gp130: | P40189 | NKRDLIKKHIWPNVPDPSKSHIAQWSPHTP | 93 |
| | Box 1 underlined | | PRHNFNSKDQMYSDGNFTDVSVVEIEANDK | |
| | Box 2 bold underlined | | KPFPEDLKSLDLFKKEKINTEGHSSGIGGS | |
| | SHP-2 and STAT3 binding sites | | SCMSSSRPSISSSDENESSQNTSSTVQYST | |
| | underlined, tyrosines bold | | VVHSGYRHQVPSVQVFSRSESTQPLLDSEE | |
| | *stop codon | | RPEDLQLVDHVDGGDGILPRQQYFKQNCSQ | |
| | | | HESSPDISHFERSKQVSSVNEEDFVRLKQQ | |
| | | | ISDHISQSCGSGQMKMFQEVSAADAFGPGT | |
| | | | EGQVERFETVGMEAATDEGMPKSYLPQTVR | |
| | | | QGGYMPQ* | |
| GEPOR | EPOR: | P19235 | HRRALKQKIWPGIPSPESEFEGLFTTHKGN | 94 |
| | Box 1 underlined | | FQLWLYQNDGCLWWSPCTPFTEDPPASLEV | |
| | Box 2 bold underlined | | LSERCWGTMQAVEPGTDDEGPLLEPVGSEH | |
| | SHP-1 & 2, | | AQDTYLVLDKWLLPRNPPSEDLPGPGGSVD | |
| | STAT5 binding sites bold | | IVAMDEGSEASSCSSALASKPSPEGASAAS | |
| | underlined | | FEYTILDPSSQLLRPWTLCPELPPTPPHLK | |
| | *stop codon | | YLYLVVSDSGISTDYSSGDSQGAQGGLSDG | |
| | | | PYSNPYENSLIPAAEPLPPSYVACS* | |
| GIFNAR2 | G-CSFR: | Q99062 | SPNRKNPLWPSVPDPAHSSLGSWVPTIMEE | 95 |
| | Box 1 underlined | | DAFQLPGLGTPPITKLTVLEEDEKKPVPWE | |
| | Box 2 bold underlined | | SHNSS | |
| | IFNAR2: | P48551 | KKKVWDYNYDDESDSDTEAAPRTSGGGYTM | 96 |
| | STAT1 & 2 binding sites bold | | HGLTVRPLGQASATSTESQLIDPESEEEPD | |
| | underlined | | LPEVDVELPTMPKDSPQQLELLSGPCERRK | |
| | *stop codon | | SPLQDPFPEEDYSSTEGSGGRITFNVDLNS | |
| | | | VFLRVLDDEDSDDLEAPLMLSSHLEEMVDP | |
| | | | EDPDNVQSNHLLASGEGTQPTFPSPSSEGL | |
| | | | WSEDAPSDQSDTSESDVDLGDGYIMR* | |
| GIFNGR1 | IFNγR2: | P38484 | KYRGLIKYWFHTPPSIPLQIEEYLKDPTQP | 97 |
| | Box 1 underlined | | ILEALDKDSSPKDDVWDSVSIISFPEKEQE | |
| | Box 2 bold underlined | | DVLQTL | |
| | IFNγR1: | P15260 | EGQELITVIKAPTSFGYDKPHVLVDLLVDD | 98 |
| | STAT1 binding site bold | | S* | |
| | underlined | | | |
| | *stop codon | | | |
| GIFNGR2 | G-CSFR: | Q99062 | SPNRKNPLWPSVPDPAHSSLGSWVPTIMEE | 99 |
| | Box 1 underlined | | DAFQLPGLGTPPITKLTVLEEDEKKPVPWE | |
| | Box 2 bold underlined | | SHNS | |
| | IFNγR1: | | | |
| | STAT1 binding site bold | P15260 | TEGQELITVIKAPTSFGYDKPHVLVDLLVD | 100 |
| | underlined | | DS* | |
| | *stop codon | | | |

TABLE 24
DOMAIN COMPOSITION OF CHIMERIC CYTOKINE RECEPTORS

| | Signal Peptide | ECD | TMD | Box1/2 | ICD | STAT | Other |
|---|---|---|---|---|---|---|---|
| Name | (SEQ ID) | (SEQ ID) | (SEQ ID) | (SEQ ID) | (SEQ ID) | site(s) | site(s) |
| G2R-3 | G-CSFR (11) | G-CSFR_134 (86) | G-CSFR (18) | G-CSFR (90) | IL-2Rβ (91) | 5 | Shc |
| G4R | G-CSFR (11) | G-CSFR_134 (86) | G-CSFR (18) | G-CSFR (90) | IL-4Rα (92) | 6 | IRS-1& 2 |
| G6R | G-CSFR (11) | G-CSFR_134 (86) | G-CSFR (18) | gp130 (93) | gp130 (93) | 3 | SHP-2 |
| GEPOR | G-CSFR (11) | G-CSFR_134 (86) | G-CSFR (18) | EPOR (94) | EPOR (94) | 5 | SHP-1 & 2 |
| GIFNAR | G-CSFR (11) | G-CSFR_134 (86) | G-CSFR (18) | G-CSFR (95) | IFNAR2 (96) | 1, 2 | |
| GIFNGR-1 | G-CSFR (11) | G-CSFR_307 (87) | G-CSFR (18) | IFNγR2 (97) | IFNγR1 (98) | 1 | |
| GIFNGR-2 | G-CSFR (11) | G-CSFR_307 (87) | G-CSFR (18) | G-CSFR (99) | IFNγR1 (100) | 1 | |

TABLE 25
MUTATIONS IN G-CSF AND G-CSFR VARIANTS

| | | Corresponding | |
|---|---|---|---|
| G-CSF | | G-CSFR | Corresponding |
| Variant | G-CSF | Variant | G-CSFR |
| Number | Mutations | Number | Mutations |
| 130a1 | E46R_L108K_D112R_E122K | 134 | R41E_R141E_R167D |
| 307 | S12E_K16D_E19K_E46R | 307 | R41E_D197K_D200K_R288E |

TABLE 26
PROTEIN SEQUENCE OF HUMAN CYTOKINE RECEPTORS

| | | | | SEQ ID |
|---|---|---|---|---|
| Protein | Uniprot ID | Domains | Polypeptide Sequence | NO. |
| IL-2Rβ/ | P14784 | Signal peptide | MAAPALSWRLPLLILLLPLATSWASAAVNGTSQFTCFYNSRANISCV | 111 |
| IL-15Rβ | | ECD | WSQDGALQDTSCQVHAWPDRRRWNQTCELLPVSQASWACNLILG | |
| | | ITM | APDSQKLTTVDIVTLRVLCREGVRWRVMAIQDFKPFENLRLMAPISL | |
| | | ICD | QVVHVETHRCNISWEISQASHYFERHLEFEARTLSPGHTWEEAPLLTL | |
| | | | KQKQEWICLETLTPDTQYEFQVRVKPLQGEFTTWSPWSQPLAFRTK | |
| | | | PAALGKDTIPWLGHLLVGLSGAFGFIILVYLLINCRNTGPWLKKVLKC | |
| | | | NTPDPSKFFSQLSSEHGGDVQKWLSSPFPSSSFSPGGLAPEISPLEVLE | |
| | | | RDKVTQLLLQQDKVPEPASLSSNHSLTSCFTNQGYFFFHLPDALEIEA | |
| | | | CQVYFTYDPYSEEDPDEGVAGAPTGSSPQPLQPLSGEDDAYCTFPSR | |
| | | | DDLLLFSPSLLGGPSPPSTAPGGSGAGEERMPPSLQERVPRDWDPQ | |
| | | | PLGPPTPGVPDLVDFQPPPELVLREAGEEVPDAGPREGVSFPWSRPP | |
| | | | GQGEFRALNARLPLNTDAYLSLQELQGQDPTHLV | |
| IL-2Rγ_{c} | P31785 | Signal peptide | MLKPSLPFTSLLFLQLPLLGVGLNTTILTPNGNEDTTADFFLTTMPTDS | 112 |
| | | ECD | LSVSTLPLPEVQCFVFNVEYMNCTWNSSSEPQPTNLTLHYWYKNSD | |
| | | TM | NDKVQKCSHYLFSEEITSGCQLQKKEIHLYQTFVVQLQDPREPRRQA | |
| | | ICD | TQMLKLQNLVIPWAPENLTLHKLSESQLELNWNNRFLNHCLEHLVQ | |
| | | | YRTDWDHSWTEQSVDYRHKFSLPSVDGQKRYTFRVRSRFNPLCGSA | |
| | | | QHWSEWSHPIHWGSNTSKENPFLFALEAVVISVGSMGLIISLLCVYF | |
| | | | WLERTMPRIPTLKNLEDLVTEYHGNFSAWSGVSKGLAESLQPDYSER | |
| | | | LCLVSEIPPKGGALGEGPGASPCNQHSPYWAPPCYTLKPET | |
| IL-4Rα | P24394 | Signal peptide | MGWLCSGLLFPVSCLVLLQVASSGNMKVLQEPTCVSDYMSISTCEW | 113 |
| | | ECD | KMNGPTNCSTELRLLYQLVFLLSEAHTCIPENNGGAGCVCHLLMDD | |
| | | TM | VVSADNYTLDLWAGQQLLWKGSFKPSEHVKPRAPGNLTVHTNVSD | |
| | | ICD | TLLLTWSNPYPPDNYLYNHLTYAVNIWSENDPADFRIYNVTYLEPSLR | |
| | | | IAASTLKSGISYRARVRAWAQCYNTTWSEWSPSTKWHNSYREPFEQ | |
| | | | HLLLGVSVSCIVILAVCLLCYVSITKIKKEWWDQIPNPARSRLVAIIIQD | |
| | | | AQGSQWEKRSRGQEPAKCPHWKNCLTKLLPCFLEHNMKRDEDPHK | |
| | | | AAKEMPFQGSGKSAWCPVEISKTVLWPESISVVRCVELFEAPVECEE | |
| | | | EEEVEEEKGSFCASPESSRDDFQEGREGIVARLTESLFLDLLGEENGGF | |
| | | | CQQDMGESCLLPPSGSTSAHMPWDEFPSAGPKEAPPWGKEQPLHL | |
| | | | EPSPPASPTQSPDNLTCTETPLVIAGNPAYRSFSNSLSQSPCPRELGPD | |
| | | | PLLARHLEEVEPEMPCVPQLSEPTTVPQPEPETWEQILRRNVLQHGA | |
| | | | AAAPVSAPTSGYQEFVHAVEQGGTQASAVVGLGPPGEAGYKAFSSL | |
| | | | LASSAVSPEKCGFGASSGEEGYKPFQDLIPGCPGDPAPVPVPLFTFGL | |
| | | | DREPPRSPQSSHLPSSSPEHLGLEPGEKVEDMPKPPLPQEQATDPLV | |
| | | | DSLGSGIVYSALTCHLCGHLKQCHGQEDGGQTPVMASPCCGCCCG | |
| | | | DRSSPPTTPLRAPDPSPGGVPLEASLCPASLAPSGISEKSKSSSSFHPA | |
| | | | PGNAQSSSQTPKIVNFVSVGPTYMRVS | |
| IL-9R | Q01113 | Signal peptide | MGLGRCIWEGWTLESEALRRDMGTWLLACICICTCVCLGVSVTGEG | 114 |
| | | ECD | QGPRSRTFTCLTNNILRIDCHWSAPELGQGSSPWLLFTSNQAPGGT | |
| | | TM | HKCILRGSECTVVLPPEAVLVPSDNFTITFHHCMSGREQVSLVDPEYL | |
| | | ICD | PRRHVKLDPPSDLQSNISSGHCILTWSISPALEPMTTLLSYELAFKKQE | |
| | | | EAWEQAQHRDHIVGVTWLILEAFELDPGFIHEARLRVQMATLEDDV | |
| | | | VEEERYTGQWSEWSQPVCFQAPQRQGPLIPPWGWPGNTLVAVSIF | |
| | | | LLLTGPTYLLFKLSPRVKRIFYQNVPSPAMFFQPLYSVHNGNFQTWM | |
| | | | GAHGAGVLLSQDCAGTPQGALEPCVQEATALLTCGPARPWKSVALE | |
| | | | EEQEGPGTRLPGNLSSEDVLPAGCTEWRVQTLAYLPQEDWAPTSLT | |
| | | | RPAPPDSEGSRSSSSSSSSNNNNYCALGCYGGWHLSALPGNTQSSG | |
| | | | PIPALACGLSCDHQGLETQQGVAWVLAGHCQRPGLHEDLQGMLLP | |
| | | | SVLSKARSWTF | |
| IL-12Rβ₂ | Q99665 | Signal peptide | MAHTFRGCSLAFMFIITWLLIKAKIDACKRGDVTVKPSHVILLGSTVNI | 115 |
| | | ECD | TCSLKPRQGCFHYSRRNKLILYKFDRRINFHHGHSLNSQVTGLPLGTT | |
| | | TM | LFVCKLACINSDEIQICGAEIFVGVAPEQPQNLSCIQKGEQGTVACTW | |
| | | ICD | ERGRDTHLYTEYTLQLSGPKNLTWQKQCKDIYCDYLDFGINLTPESPE | |
| | | | SNFTAKVTAVNSLGSSSSLPSTFTFLDIVRPLPPWDIRIKFQKASVSRCT | |
| | | | LYWRDEGLVLLNRLRYRPSNSRLWNMVNVTKAKGRHDLLDLKPFTE | |
| | | | YEFQISSKLHLYKGSWSDWSESLRAQTPEEEPTGMLDVWYMKRHID | |
| | | | YSRQQISLFWKNLSVSEARGKILHYQVTLQELTGGKAMTQNITGHTS | |
| | | | WTTVIPRTGNWAVAVSAANSKGSSLPTRINIMNLCEAGLLAPRQVS | |
| | | | ANSEGMDNILVTWQPPRKDPSAVQEYVVEWRELHPGGDTQVPLN | |
| | | | WLRSRPYNVSALISENIKSYICYEIRVYALSGDQGGCSSILGNSKHKAPL | |
| | | | SGPHINAITEEKGSILISWNSIPVQEQMGCLLHYRIYWKERDSNSQPQ | |
| | | | LCEIPYRVSQNSHPINSLQPRVTYVLWMTALTAAGESSHGNEREFCL | |
| | | | QGKANWMAFVAPSICIAIIMVGIFSTHYFQQKVFVLLAALRPQWCSR | |
| | | | EIPDPANSTCAKKYPIAEEKTQLPLDRLLIDWPTPEDPEPLVISEVLHQ | |
| | | | VTPVFRHPPCSNWPQREKGIQGHQASEKDMMHSASSPPPPRALQA | |
| | | | ESRQLVDLYKVLESRGSDPKPENPACPWTVLPAGDLPTHDGYLPSNI | |
| | | | DDLPSHEAPLADSLEELEPQHISLSVFPSSSLHPLTFSCGDKLTLDQLK | |
| | | | MRCDSLML | |
| IL-21R | Q9HBE5 | Signal peptide | MPRGWAAPLLLLLLQGGWGCPDLVCYTDYLQTVICILEMWNLHPST | 116 |
| | | ECD | LTLTWQDQYEELKDEATSCSLHRSAHNATHATYTCHMDVFHFMAD | |
| | | TM | DIFSVNITDQSGNYSQECGSFLLAESIKPAPPFNVTVTFSGQYNISWRS | |
| | | ICD | DYEDPAFYMLKGKLQYELQYRNRGDPWAVSPRRKLISVDSRSVSLLP | |
| | | | LEFRKDSSYELQVRAGPMPGSSYQGTWSEWSDPVIFQTQSEELKEG | |
| | | | WNPHLLLLLLLVIVFIPAFWSLKTHPLWRLWKKIWAVPSPERFFMPLY | |
| | | | KGCSGDFKKWVGAPFTGSSLELGPWSPEVPSTLEVYSCHPPRSPAKR | |
| | | | LQLTELQEPAELVESDGVPKPSFWPTAQNSGGSAYSEERDRPYGLVSI | |
| | | | DTVTVLDAEGPCTWPCSCEDDGYPALDLDAGLEPSPGLEDPLLDAGT | |
| | | | TVLSCGCVSAGSPGLGGPLGSLLDRLKPPLADGEDWAGGLPWGGRS | |
| | | | PGGVSESEAGSPLAGLDMDTFDSGFVGSDCSSPVECDFTSPGDEGP | |
| | | | PRSYLRQWVVIPPPLSSPGPQAS | |
| gp130 | P40189 | Signal peptide | MLTLQTWLVQALFIFLTTESTGELLDPCGYISPESPVVQLHSNFTAVC | |
| | | ECD | VLKEKCMDYFHVNANYIVWKTNHFTIPKEQYTIINRTASSVTFTDIASL | |
| | | TM | NIQLTCNILTFGQLEQNVYGITIISGLPPEKPKNLSCIVNEGKKMRCEW | |
| | | ICD | DGGRETHLETNFTLKSEWATHKFADCKAKRDTPTSCTVDYSTVYFVN | |
| | | | IEVWVEAENALGKVTSDHINFDPVYKVKPNPPHNLSVINSEELSSILKL | |
| | | | TWTNPSIKSVIILKYNIQYRTKDASTWSQIPPEDTASTRSSFTVQDLKP | |
| | | | FTEYVFRIRCMKEDGKGYWSDWSEEASGITYEDRPSKAPSFWYKIDP | |
| | | | SHTQGYRTVQLVWKTLPPFEANGKILDYEVTLTRWKSHLQNYTVNA | |
| | | | TKLTVNLTNDRYLATLTVRNLVGKSDAAVLTIPACDFQATHPVMDLK | |
| | | | AFPKDNMLWVEWTTPRESVKKYILEWCVLSDKAPCITDWQQEDGT | |
| | | | VHRTYLRGNLAESKCYLITVTPVYADGPGSPESIKAYLKQAPPSKGPTV | 117 |
| | | | RTKKVGKNEAVLEWDQLPVDVQNGFIRNYTIFYRTIIGNETAVNVDS | |
| | | | SHTEYTLSSLTSDTLYMVRMAAYTDEGGKDGPEFTFTTPKFAQGEIEA | |
| | | | IVVPVCLAFLLTTLLGVLFCFNKRDLIKKHIWPNVPDPSKSHIAQWSP | |
| | | | HTPPRHNFNSKDQMYSDGNFTDVSVVEIEANDKKPFPEDLKSLDLFK | |
| | | | KEKINTEGHSSGIGGSSCMSSSRPSISSSDENESSQNTSSTVQYSTVVH | |
| | | | SGYRHQVPSVQVFSRSESTQPLLDSEERPEDLQLVDHVDGGDGILPR | |
| | | | QQYFKQNCSQHESSPDISHFERSKQVSSVNEEDFVRLKQQISDHISQS | |
| | | | CGSGQMKMFQEVSAADAFGPGTEGQVERFETVGMEAATDEGMP | |
| | | | KSYLPQTVRQGGYMPQ | |

TABLE 27
ICD ADAPTOR PROTEIN BINDING SITES.

| | | | | SEQ |
|---|---|---|---|---|
| Receptor | | Binding | | ID |
| name | Uniprot ID | site | Polypeptide Sequence | NO. |
| IL-7Rα | P16871 | Box 1 | KRIKPIVWPSLPDHKKTLEHL | 118 |
| | | Box 2 - 1 | KKPRKNLNVSFNPESF | 119 |
| | | Box 2 - 2 | PESFLDCQIHRVDDIQ | 120 |
| IL-2Rβ | P14784 | Box 1 | PWLKKVLKCNTPDPSKFFSQL | 121 |
| | | Box 2 | APEISPLEVLERDKVT | 122 |
| | | Shc | TSCFTNQGYFFFHLPDA | 123 |
| | | STAT5 | RLPLNTDAYLSLQELQG | 124 |
| IL-2Rγ_{c} | P31785 | Box 1 | ERTMPRIPTLKNLEDL | 125 |
| | | Box 2 | PDYSERLCLVSEIPPK | 126 |
| IL-4Rα | P24394 | Box 1 | KIKKEWWDQIPNPARSRLVA | 127 |
| | | Box 2 | LTKLLPCFLEHNMKRD | 128 |
| | | IRS-2 | LVIAGNPAYRSFSNSLS | 129 |
| | | STAT6 - 1 | LGPPGEAGYKAFSSLLA | 130 |
| | | STAT6 - 2 | GASSGEEGYKPFQDLIP | 131 |
| IL-9R | Q01113 | Box 1 | SPRVKRIFYQNVPSPAMFFQP | 132 |
| | | Box 2 - 1 | GAHGAGVLLSQDCAGT | 133 |
| | | Box 2 - 2 | GTPQGALEPCVQEATA | 134 |
| IL-12Rβ₂ | Q99665 | STAT4 | AGDLPTHDGYLPSNIDDLPSHEAPLA | 135 |
| IL-21R | Q9HBE5 | Box 1 | LWRLWKKIWAVPSPERFFMPL | 136 |
| | | Box 2 - 1 | PFTGSSLELGPWSPEV | 137 |
| | | Box 2 - 2 | PEVPSTLEVYSCHPPR | 138 |
| | | STAT3 | GDEGPPRSYLRQWVVIP | 139 |
| gp130 | P40189 | Box 1 | DLIKKHIWPNVPDPSKSHIAQ | 140 |
| | | Box 2 | DGNFTDVSVVEIEAND | 141 |
| | | SHP-2 | QNTSSTVQYSTVVHSG | 142 |
| | | STAT3 - 1 | STVVHSGYRHQVPSVQ | 143 |
| | | STAT3 - 2 | DGILPRQQYFKQNCSQHESS | 144 |
| | | STAT3 - 3 | EGMPKSYLPQTVRQ | 145 |
| | | STAT3 - 4 | TVRQGGYMPQ | 146 |

TABLE 28
EXAMPLES OF ENGINEERED T CELL RECEPTORS (ETCRS)

| | | | | SEQ |
|---|---|---|---|---|
| Chimeric | | | | ID |
| receptor | Receptor name: domains | Patent | Polypeptide Sequence | NO. |
| HPV 16 E7 | LVL-modified chimeric TCR: | US | MAPGLLCWALLCLLGAGLVDAGVTQSPTHLIKTRGQ | 147 |
| eTCR | β chain Variable region - | 10174098 | QVTLRCSPKSGHDTVSWYQQALGQGPQFIFQYYEE | |
| | Murine β chain Constant | B2 | EERQRGNFPDRFSGHQFPNYSSELNVNALLLGDSAL | |
| | region - Linker peptide (P2A) - | | YLCASSLGWRGGRYNEQFFGPGTRLTVLEDLRNVTP | |
| | Human α chain Variable | | PKVSLFEPSKAEIANKQKATLVCLARGFFPDHVELSW | |
| | region - Murine a chain | | WVNGKEVHSGVSTDPQAYKESNYSYCLSSRLRVSAT | |
| | Constant region - Murine α | | FWHNPRNHFRCQVQFHGLSEEDKWPEGSPKPVTQ | |
| | chain TMD | | NISAEAWGRADCGITSASYQQGVLSATILYEILLGKAT | |
| | | | LYAVLVSTLVVMAMVKRKNSRAKRSGSGATNFSLLK | |
| | | | QAGDVEENPGPMWGVFLLYVSMKMGGTTGQNID | |
| | | | QPTEMTATEGAIVQINCTYQTSGFNGLFWYQQHAG | |
| | | | EAPTFLSYNVLDGLEEKGRFSSFLSRSKGYSYLLLKELQ | |
| | | | MKDSASYLCASVDGNNRLAFGKGNQVVVIPNIQNP | |
| | | | EPAVYQLKDPRSQDSTLCLFTDFDSQINVPKTMESG | |
| | | | TFITDKTVLDMKAMDSKSNGAIAWSNQTSFTCQDIF | |
| | | | KETNATYPSSDVPCDATLTEKSFETDMNLNFQNLLVI | |
| | | | VLRILLLKVAGFNLLMTLRLWSS | |
| | Cys-substituted, LVL- | | MAPGLLCWALLCLLGAGLVDAGVTQSPTHLIKTRGQ | 148 |
| | modified chimeric TCR: | | QVTLRCSPKSGHDTVSWYQQALGQGPQFIFQYYEE | |
| | β chain Variable region - | | EERQRGNFPDRFSGHQFPNYSSELNVNALLLGDSAL | |
| | Murine β chain Constant | | YLCASSLGWRGGRYNEQFFGPGTRLTVLEDLRNVTP | |
| | region - Linker peptide (P2A) - | | PKVSLFEPSKAEIANKQKATLVCLARGFFPDHVELSW | |
| | Human α chain Variable | | WVNGKEVHSGVCTDPQAYKESNYSYCLSSRLRVSAT | |
| | region - Murine a chain | | FWHNPRNHFRCQVQFHGLSEEDKWPEGSPKPVTQ | |
| | Constant region - Murine α | | NISAEAWGRADCGITSASYQQGVLSATILYEILLGKAT | |
| | chain TMD | | LYAVLVSTLVVMAMVKRKNSRAKRSGSGATNFSLLK | |
| | | | QAGDVEENPGPMWGVFLLYVSMKMGGTTGQNID | |
| | | | QPTEMTATEGAIVQINCTYQTSGFNGLFWYQQHAG | |
| | | | EAPTFLSYNVLDGLEEKGRFSSFLSRSKGYSYLLLKELQ | |
| | | | MKDSASYLCASVDGNNRLAFGKGNQVVVIPNIQNP | |
| | | | EPAVYQLKDPRSQDSTLCLFTDFDSQINVPKTMESG | |
| | | | TFITDKCVLDMKAMDSKSNGAIAWSNQTSFTCQDIF | |
| | | | KETNATYPSSDVPCDATLTEKSFETDMNLNFQNLLVI | |
| | | | VLRILLLKVAGFNLLMTLRLWSS | |
| NY-ESO-1 | NY-ESO-1^{c259} TCR a chain | WO | METLLGLLILWLGLGWVSSKGEVTGIPAALSVPEGEN | 149 |
| eTCR | | 2020/ | LVLNCSFTDSAIYNLGWFRGDPGKGLTSLLLIGSSGRE | |
| | | 049496 | GTSGRLNASLDKSSGRSTLYIAASGPGDSATYLCAVR | |
| | | A1 | PLYGGSYIPTFGRGTSLIVHPYIONPDPAVYOLRDSKS | |
| | | | SDKSVCLFTDFDSOTNVSOSKDSDVYITDKTVLDMR | |
| | | | SMDFKSNSAVAWSNKSDFACANAFNNSIIPEDTFFP | |
| | | | SPESSCDVKLVEKSFETDTNLNFCNLSVIGFRILLLKVA | |
| | | | GFNLLMTLRLWSS | |
| | NY-ESO-1 ^{c259} TCR b chain | | RMSIGLLCCAALSLLWAGPVNAGVTGTPKFGVLKTG | 150 |
| | | | GSMTLGCAGDMNHEYMSWYRGDPGMGLRLIHYS | |
| | | | VGAGITDGGEVPNGYNVSRSTTEDFPLRLLSAAPSGT | |
| | | | SVYFCASSYVGNTGELFFGEGSRLTVLEDLKNVFPPE | |
| | | | VAVFEPSEAEISHTCKATLVCLATGFYPDHVELSWW | |
| | | | VNGKEVHSGVSTDPGPLKEGPALNDSRYCLSSRLRVS | |
| | | | ATFWGNPRNHFRCGVGFYGLSENDEWTODRAKPV | |
| | | | TOIVSAEAWGRADCGFTSESYOOGVLSATILYEILLG | |
| | | | KATLYAVLVSALVLMAMVKRKDSRG | |

TABLE 29
EXAMPLES OF CHIMERIC ANTIGEN RECEPTORS (CARS)

| | | | | SEQ |
|---|---|---|---|---|
| Chimeric | Receptor name | | | ID |
| receptor | Domains | Patent | Polypeptide Sequence | NO. |
| Second | CTL019: | WO20141 | MALPVTALLLPLALLLHAARPDIQMTQTTSSLSASLGD | 151 |
| generation | Leader - VH - Linker - VL - CD8 | 53270A1 | RVTISCRASQDISKYLNWYQQKPDGTVKLLIYHTSRIH | |
| 4-1BB | hinge - CD8 TM - 4-1BB - CD3ζ | | SGVPSRFSGSGSGTDYSLTISNLEQEDIATYFCQQGNT | |
| containing | | | LPYTFGGGTKLEITGGGGSGGGGSGGGGSEVKLQES | |
| anti-CD19 | | | GPGLVAPSQSLSVTCTVSGVSLPDYGVSWIRQPPRKG | |
| CAR | | | LEWLGVIWGSETTYYNSALKSRITIIKDNSKSQVFLKM | |
| | | | NSLQTDDTAIYYCAKHYYYGGSYAMDYWGQGTSVTV | |
| | | | SSTTTPAPRPPTPAPTIASQPLSLRPEACRPAAGGAVH | |
| | | | TRGLDFACDIYIWAPLAGTCGVLLLSLVITLYCKRGRKK | |
| | | | LLYIFKQPFMRPVQTTQEEDGCSCRFPEEEEGGCELRV | |
| | | | KFSRSADAPAYKQGQNQLYNELNLGRREEYDVLDKR | |
| | | | RGRDPEMGGKPRRKNPQEGLYNELQKDKMAEAYSEI | |
| | | | GMKGERRRGKGHDGLYQGLSTATKDTYDALHMQAL | |
| | | | PPR | |
| | Leader | | MALPVTALLLPLALLLHAARP | 152 |
| | VH | | DIQMTQTTSSLSASLGDRVTISCRASQDISKYLNWYQ | 153 |
| | | | QKPDGTVKLLIYHTSRIHSGVPSRFSGSGSGTDYSLTIS | |
| | | | NLEQEDIATYFCQQGNTLPYTFGGGTKLEIT | |
| | Linker | | GGGGSGGGGSGGGGS | 154 |
| | VL | | EVKLQESGPGLVAPSQSLSVTCTVSGVSLPDYGVSWI | 155 |
| | | | RQPPRKGLEWLGVIWGSETTYYNSALKSRITIIKDNSK | |
| | | | SQVFLKMNSLQTDDTAIYYCAKHYYYGGSYAMDYW | |
| | | | GQGTSVTVSS | |
| | CD8 hinge | | TTTPAPRPPTPAPTIASQPLSLRPEACRPAAGGAVHTR | 156 |
| | | | GLDFACD | |
| | CD8 TM | | IYIWAPLAGTCGVLLLSLVITLYC | 157 |
| | 4-1BB | | KRGRKKLLYIFKQPFMRPVQTTQEEDGCSCRFPEEEE | 158 |
| | | | GGCEL | |
| | CD3ζ | | RVKFSRSADAPAYKQGQNQLYNELNLGRREEYDVLD | 159 |
| | | | KRRGRDPEMGGKPRRKNPQEGLYNELQKDKMAEAY | |
| | | | SEIGMKGERRRGKGHDGLYQGLSTATKDTYDALHM | |
| | | | QALPPR | |
| First | P4-z: | US | MALPVTALLLPLALLLHAARPGSQVQLQQSGPGLVTP | 160 |
| generation | Leader - VH - Linker - VL - CD8 | 9272002 | SQTLSLTCAISGDSVSSNSATWNWIRQSPSRGLEWLG | |
| anti- | hinge - CD8 TM - CD3ζ | B2 | RTYYRSKWYNDYAVSVKSRMSINPDTSKNQFSLQLNS | |
| mesothelin | | | VTPEDTAVYYCARGMMTYYYGMDVWGQGTTVTVS | |
| CAR | | | SGILGSGGGGSGGGGSGGGGSQPVLTQSSSLSASPG | |
| | | | ASASLTCTLRSGINVGPYRIYWYQQKPGSPPQYLLNYK | |
| | | | SDSDKQQGSGVPSRFSGSKDASANAGVLLISGLRSED | |
| | | | EADYYCMIWHSSAAVFGGGTQLTVLSASTTTPAPRPP | |
| | | | TPAPTIASRPLSLRPEACRPAAGGAVHTRGLDFACDIYI | |
| | | | WAPLAGTCGVLLLSLVITLYCRVKFSRSADAPAYQNG | |
| | | | QNQLYNELNLGRREEYDVLDKRRGRDPEMGGKPRRK | |
| | | | NPQEGLYNELNKDKMAEAYSEIGMKGERRRGKGHD | |
| | | | GLYQGLSTATKDTYDALHMQALPPR | |
| | Leader | | MALPVTALLLPLALLLHAARPGSQVQLQQSGPGL | 161 |
| | VH | | VTPSQTLSLTCAISGDSVSSNSATWNWIRQSPSRGLE | 162 |
| | | | WLGRTYYRSKWYNDYAVSVKSRMSINPDTSKNQFSL | |
| | | | QLNSVTPEDTAVYYCARGMMTYYYGMDVWGQGTT | |
| | | | VTVSSGIL | |
| | Linker | | GSGGGGSGGGGSGGGGS | 163 |
| | VL | | QPVLTQSSSLSASPGASASLTCTLRSGINVGPYRIYWY | 164 |
| | | | QQKPGSPPQYLLNYKSDSDKQQGSGVPSRFSGSKDA | |
| | | | SANAGVLLISGLRSEDEADYYCMIWHSSAAVFGGGT | |
| | | | QLTVLSAS | |
| | CD8 hinge | | TTTPAPRPPTPAPTIASRPLSLRPEACRPAAGGAVHTR | 165 |
| | | | GLDFACD | |
| | CD8 TM | | IYIWAPLAGTCGVLLLSLVITLYC | 166 |
| | CD3ζ | | RVKFSRSADAPAYQNGQNQLYNELNLGRREEYDVLD | 167 |
| | | | KRRGRDPEMGGKPRRKNPQEGLYNELNKDKMAEAY | |
| | | | SEIGMKGERRRGKGHDGLYQGLSTATKDTYDALHM | |
| | | | QALPPR | |
| Second | HN1scFv-28Z: | US | MVLLVTSLLLCELPHPAFLLIPDIQAQVQLVQSGAEVK | 168 |
| generation | Leader - VH - Linker - VL - CD8 | 9359447 | RPGASVQVSCRASGYSINTYYMQWVRQAPGAGLEW | |
| CD28 | hinge - CD8 TM - CD28 ICD - | B2 | MGVINPSGVTSYAQKFQGRVTLTNDTSTNTVYMQLN | |
| containing | CD3ζ | | SLTSADTAVYYCARWALWGDFGMDVWGKGTLVTV | |
| anti- | | | SSGGGGSGGGGSGGGGSDIQMTQSPSTLSASIGDRV | |
| mesothelin | | | TITCRASEGIYHWLAWYQQKPGKAPKLLIYKASSLASG | |
| CAR | | | APSRFSGSGSGTDFTLTISSLQPDDFATYYCQQYSNYP | |
| | | | LTFGGGTKLEIKRAAAIEVMYPPPYLDNEKSNGTIIHVK | |
| | | | GKHLCPSPLFPGPSKPFWVLVVVGGVLACYSLLVTVA | |
| | | | FIIFWVRSKRSRLLHSDYMNMTPRRPGPTRKHYQPYA | |
| | | | PPRDFAAYRSRVKFSRSADAPAYQQGQNQLYNELNL | |
| | | | GRREEYDVLDKRRGRDPEMGGKPRRKNPQEGLYNEL | |
| | | | QKDKMAEAYSEIGMKGERRRGKGHDGLYQGLSTATK | |
| | | | DTYDALHMQALPPR | |
| | Leader | | MVLLVTSLLLCELPHPAFLLIPDIQ | 169 |
| | VH | | AQVQLVQSGAEVKRPGASVQVSCRASGYSINTYYMQ | 170 |
| | | | WVRQAPGAGLEWMGVINPSGVTSYAQKFQGRVTLT | |
| | | | NDTSTNTVYMQLNSLTSADTAVYYCARWALWGDFG | |
| | | | MDVWGKGTLVTVSS | |
| | Linker | | GGGGSGGGGSGGGGS | 171 |
| | VL | | DIQMTQSPSTLSASIGDRVTITCRASEGIYHWLAWYQ | 172 |
| | | | QKPGKAPKLLIYKASSLASGAPSRFSGSGSGTDFTLTIS | |
| | | | SLQPDDFATYYCQQYSNYPLTFGGGTKLEIKR | |
| | CD8 hinge | | AAAIEVMYPPPYLDNEKSNGTIIHVKGKHLCPSPLFPG | 173 |
| | | | PSKP | |
| | CD8 TM | | FWVLVVVGGVLACYSLLVTVAFIIFWV | 174 |
| | CD28 ICD | | RSKRSRLLHSDYMNMTPRRPGPTRKHYQPYAPPRDF | 175 |
| | | | AAYRS | |
| | CD3ζ | | RVKFSRSADAPAYQQGQNQLYNELNLGRREEYDVLD | 176 |
| | | | KRRGRDPEMGGKPRRKNPQEGLYNELQKDKMAEAY | |
| | | | SEIGMKGERRRGKGHDGLYQGLSTATKDTYDALHM | |
| | | | QALPPR | |
| Third | HN1scFv-28BBZ: | | MVLLVTSLLLCELPHPAFLLIPDIQAQVQLVQSGAEVK | 177 |
| generation | Leader - VH - Linker - VL - | | RPGASVQVSCRASGYSINTYYMQWVRQAPGAGLEW | |
| CD28 and | CD28 hinge - CD28 TM - CD28 | | MGVINPSGVTSYAQKFQGRVTLTNDTSTNTVYMQLN | |
| 4-1BB | ICD - 4-1BB - CD3ζ | | SLTSADTAVYYCARWALWGDFGMDVWGKGTLVTV | |
| containing | | | SSGGGGSGGGGSGGGGSDIQMTQSPSTLSASIGDRV | |
| anti- | | | TITCRASEGIYHWLAWYQQKPGKAPKLLIYKASSLASG | |
| mesothelin | | | APSRFSGSGSGTDFTLTISSLQPDDFATYYCQQYSNYP | |
| CAR | | | LTFGGGTKLEIKRAAAFVPVFLPAKPTTTPAPRPPTPA | |
| | | | PTIASQPLSLRPEACRPAAGGAVHTRGLDFACDIYIWA | |
| | | | PLAGTCGVLLLSLVITLYCNHRNRSKRSRLLHSDYMN | |
| | | | MTPRRPGPTRKHYQPYAPPRDFAAYRSRFSVVKRGR | |
| | | | KKLLYIFKQPFMRPVQTTQEEDGCSCRFPEEEEGGCEL | |
| | | | RVKFSRSADAPAYQQGQNQLYNELNLGRREEYDVLD | |
| | | | KRRGRDPEMGGKPRRKNPQEGLYNELQKDKMAEAY | |
| | | | SEIGMKGERRRGKGHDGLYQGLSTATKDTYDALHM | |
| | | | QALPPR | |
| | Leader - | | MVLLVTSLLLCELPHPAFLLIPDIQ | 178 |
| | VH - | | AQVQLVQSGAEVKRPGASVQVSCRASGYSINTYYMQ | 179 |
| | | | WVRQAPGAGLEWMGVINPSGVTSYAQKFQGRVTLT | |
| | | | NDTSTNTVYMQLNSLTSADTAVYYCARWALWGDFG | |
| | | | MDVWGKGTLVTVSS | |
| | Linker - | | GGGGSGGGGSGGGGS | 180 |
| | VL - | | DIQMTQSPSTLSASIGDRVTITCRASEGIYHWLAWYQ | 181 |
| | | | QKPGKAPKLLIYKASSLASGAPSRFSGSGSGTDFTLTIS | |
| | | | SLQPDDFATYYCQQYSNYPLTFGGGTKLEIKR | |
| | CD28 hinge - | | AAAFVPVFLPAKPTTTPAPRPPTPAPTIASQPLSLRPEA | 182 |
| | | | CRPAAGGAVHTRGLDFACD | |
| | CD28 TM - | | IYIWAPLAGTCGVLLLSLVITLYCNHRN | 183 |
| | CD28 ICD - | | RSKRSRLLHSDYMNMTPRRPGPTRKHYQPYAPPRDF | 184 |
| | | | AAYRS | |
| | 4-1BB - | | RFSVVKRGRKKLLYIFKQPFMRPVQTTQEEDGCSCRF | 185 |
| | | | PEEEEGGCEL | |
| | CD37 | | RVKFSRSADAPAYQQGQNQLYNELNLGRREEYDVLD | 186 |
| | | | KRRGRDPEMGGKPRRKNPQEGLYNELQKDKMAEAY | |
| | | | SEIGMKGERRRGKGHDGLYQGLSTATKDTYDALHM | |
| | | | QALPPR | |

TABLE 30
CYTOKINES, CHEMOKINES AND OTHER SIGNALING PROTEINS:

| | | | SEQ |
|---|---|---|---|
| | | | ID |
| Cytokine | Uniprot ID | Polypeptide Sequence | NO. |
| hIL-18: | Q14116 | MAAEPVEDNCINFVAMKFIDNTLYFIAEDDENLESDYFGKLESKLSVIRNL | 102 |
| Signal peptide | | NDQVLFIDQGNRPLFEDMTDSDCRDNAPRTIFIISMYKDSQPRGMAVTI | |
| underlined | | SVKCEKISTLSCENKIISFKEMNPPDNIKDTKSDIIFFQRSVPGHDNKMQF | |
| | | ESSSYEGYFLACEKERDLFKLILKKEDELGDRSIMFTVQNED | |
| hCXCL13: | Q52X90 | MKFISTSLLLMLLVSSLSPVQGVLEVYYTSLRCRCVQESSVFIPRRFIDRIQI | 103 |
| Signal peptide | | LPRGNGCPRKEIIVWKKNKSIVCVDPQAEWIQRMMEVLRKRSSSTLPVP | |
| underlined | | VFKRKIP | |
| hIL-21: | Q9HBE4 | MRSSPGNMERIVICLMVIFLGTLVHKSSSQGQDRHMIRMRQLIDIVDQL | 104 |
| Signal peptide | | KNYVNDLVPEFLPAPEDVETNCEWSAFSCFQKAQLKSANTGNNERIINV | |
| underlined | | SIKKLKRKPPSTNAGRRQKHRLTCPSCDSYEKKPPKEFLERFKSLLQKMIH | |
| | | QHLSSRTHGSEDS | |
| hCCL3: | P10147 | MQVSTAALAVLLCTMALCNQFSASLAADTPTACCFSYTSRQIPQNFIAD | 105 |
| Signal peptide | | YFETSSQCSKPGVIFLTKRSRQVCADPSEEWVQKYVSDLELSA | |
| underlined | | | |
| hCCL4: | P13236 | MKLCVTVLSLLMLVAAFCSPALSAPMGSDPPTACCFSYTARKLPRNFVV | 106 |
| Signal peptide | | DYYETSSLCSQPAVVFQTKRSKQVCADPSESWVQEYVYDLELN | |
| underlined | | | |
| hCCL19: | Q99731 | MALLLALSLLVLWTSPAPTLSGTNDAEDCCLSVTQKPIPGYIVRNFHYLLI | 107 |
| Signal peptide | | KDGCRVPAVVFTTLRGRQLCAPPDQPWVERIIQRLQRTSAKMKRRSS | |
| underlined | | | |
| hIFNγ: | P01579 | MKYTSYILAFQLCIVLGSLGCYCQDPYVKEAENLKKYFNAGHSDVADNG | 108 |
| Signal peptide | | TLFLGILKNWKEESDRKIMQSQIVSFYFKLFKNFKDDQSIQKSVETIKEDM | |
| underlined | | NVKFFNSNKKKRDDFEKLTNYSVTDLNVQRKAIHELIQVMAELSPAAKT | |
| | | GKRKRSQMLFRGRRASQ | |
| hIFNα-1/13: | P01562 | MASPFALLMVLVVLSCKSSCSLGCDLPETHSLDNRRTLMLLAQMSRISPS | 109 |
| Signal peptide | | SCLMDRHDFGFPQEEFDGNQFQKAPAISVLHELIQQIFNLFTTKDSSAA | |
| underlined | | WDEDLLDKFCTELYQQLNDLEACVMQEERVGETPLMNADSILAVKKYF | |
| | | RRITLYLTEKKYSPCAWEVVRAEIMRSLSLSTNLQERLRRKE | |
| hIL-17: | Q16552 | MTPGKTSLVSLLLLLSLEAIVKAGITIPRNPGCPNSEDKNFPRTVMVNLNI | 110 |
| Signal peptide | | HNRNTNTNPKRSSDYYNRSTSPWNLHRNEDPERYPSVIWEAKCRHLGC | |
| underlined | | INADGNVDYHMNSVPIQQEILVLRREPPHCPNSFRLEKILVSVGCTCVTP | |
| | | IVHHVA | |
| hTNF-α: | P01357 | MSTESMIRDVELAEEALPKKTGGPQGSRRCLFLSLFSFLIVAGATTLFCLL | SEQ |
| Membrane- | | HFGVIGPQREEFPRDLSLISPLAQAVRSSSRTPSDKPVAHVVANPQAEG | ID |
| bound portion | | QLQWLNRRANALLANGVELRDNQLVVPSEGLYLIYSQVLFKGQGCPST | NO. |
| underlined | | HVLLTHTISRIAVSYQTKVNLLSAIKSPCQRETPEGAEAKPWYEPIYLGGV | 215) |
| | | FQLEKGDRLSAEINRPDYLDFAESGQVYFGIIAL | |
| hNKG2-D: | P26718 | MGWIRGRRSRHSWEMSEFHNYNLDLKKSDFSTRWQKQRCPVVKSKC | SEQ |
| ICD-TM-ECD | | RENASPFFFCCFIAVAMGIRFIIMVAIWSAVFLNSLFNQEVQIPLTESYCG | ID |
| | | PCPKNWICYKNNCYQFFDESKNWYESQASCMSQNASLLKVYSKEDQDL | NO. |
| | | LKLVKSYHWMGLVHIPTNGSWQWEDGSILSPNLLTIIEMQKGDCALYA | 216) |
| | | SSFKGYIENCSTPNTYICMQRTV | |

TABLE 31
DNA SEQUENCES FOR CONTROLLED PARACRINE SIGNALING
(CPS) EXEMPLARY CONSTRUCTS

| | | | | SEQ |
|---|---|---|---|---|
| | Ref Seq | Residue | | ID |
| Gene | ID | numbering | DNA Sequence | NO. |
| CAR_P2A_ | | 1-4758 | CCCGCCAGGATCCTGACGCGTATGGTGCTGCTGGTC | SEQ |
| G12- | | | ACCAGCTTACTCCTGTGCGAGCTCCCCCATCCTGCGT | ID |
| 2R1- | | | TCTTGCTGATCCCCGACATCCAGCAGGTGCAGCTGC | NO. |
| 134_T2A_ | | | AACAGTCGGGCCCCGAGTTAGAAAAACCCGGCGCCT | 217) |
| hIL-18 | | | CCGTGAAGATCTCGTGTAAAGCTTCGGGCTACTCGT | |
| | | | TTACCGGCTACACCATGAATTGGGTTAAGCAGAGCC | |
| | | | ATGGAAAGTCTCTTGAATGGATAGGCCTGATTACGC | |
| | | | CTTATAATGGGGCCTCGAGCTACAACCAGAAATTTC | |
| | | | GTGGTAAGGCAACTCTGACTGTAGACAAGTCGTCCT | |
| | | | CCACCGCATACATGGATCTTCTGAGCCTAACCTCAG | |
| | | | AAGACAGCGCAGTCTACTTCTGCGCCCGTGGTGGTT | |
| | | | ACGATGGCCGCGGATTCGACTATTGGGGCCAGGGCA | |
| | | | CCACCGTCACCGTGAGCTCCGGCGGTGGCGGGTCCG | |
| | | | GTGGCGGCGGCTCAAGCGGAGGAGGTAGTGACATC | |
| | | | GAGTTGACCCAGTCGCCTGCCATCATGTCCGCATCC | |
| | | | CCCGGCGAGAAGGTGACGATGACGTGTTCTGCTTCC | |
| | | | AGCTCTGTATCGTACATGCACTGGTACCAACAGAAG | |
| | | | TCCGGCACCTCCCCCAAGCGTTGGATCTACGACACT | |
| | | | TCCAAGCTGGCGTCTGGCGTCCCTGGGCGCTTTTCAG | |
| | | | GTAGCGGGTCCGGCAATAGCTATTCCTTGACGATCA | |
| | | | GTAGCGTGGAGGCTGAGGATGATGCCACCTACTACT | |
| | | | GCCAGCAGTGGAGCAAGCACCCGCTCACCTTCGGCG | |
| | | | CCGGGACCAAGCTTGAAATCAAGGCGGCGGCGATC | |
| | | | GAGGTGATGTATCCTCCTCCCTACCTGGACAACGAG | |
| | | | AAATCCAATGGCACTATCATCCACGTGAAGGGGAAG | |
| | | | CACCTGTGCCCGAGCCCCCTGTTCCCCGGCCCTTCCA | |
| | | | AGCCTTTTTGGGTGCTTGTTGTGGTCGGCGGCGTGCT | |
| | | | GGCCTGCTACTCTCTGCTGGTAACCGTAGCGTTCATC | |
| | | | ATCTTCTGGGTGCGATCTAAGCGGTCCCGGCTGTTGC | |
| | | | ACAGTGACTACATGAACATGACCCCGCGCCGCCCCG | |
| | | | GCCCAACCCGCAAGCACTACCAGCCGTACGCTCCAC | |
| | | | CTCGCGACTTTGCCGCCTACAGGTCGCGAGTGAAAT | |
| | | | TTTCGCGCTCCGCGGACGCGCCGGCCTATCAGCAGG | |
| | | | GCCAGAACCAGCTTTACAACGAGTTGAACCTCGGGC | |
| | | | GCAGGGAGGAGTACGACGTGCTGGATAAACGTAGA | |
| | | | GGTCGGGACCCGGAGATGGGCGGCAAACCACGCCG | |
| | | | CAAGAACCCTCAGGAAGGCCTGTACAACGAGCTCCA | |
| | | | GAAGGACAAAATGGCTGAGGCGTACTCGGAGATTG | |
| | | | GGATGAAAGGCGAGCGGCGCCGCGGCAAGGGCCAC | |
| | | | GATGGCCTGTACCAGGGGCTATCTACCGCTACAAAG | |
| | | | GACACCTACGACGCCCTACATATGCAGGCCCTGCCA | |
| | | | CCGCGTGGCTCTGGCGCAACCAACTTCTCGCTTCTGA | |
| | | | AGCAGGCCGGTGATGTGGAGGAAAACCCAGGTCCT | |
| | | | ATGGCCCGCCTGGGCAACTGCTCGCTGACCTGGGCG | |
| | | | GCCCTGATCATCCTGCTGCTGCCCGGCTCTCTGGAGG | |
| | | | AGTGTGGCCATATTTCCGTGTCAGCTCCAATCGTGCA | |
| | | | CCTGGGCGACCCCATCACCGCTTCTTGCATCATCAA | |
| | | | GCAGAACTGCTCTCACCTCGACCCTGAGCCCCAAAT | |
| | | | ACTTTGGGAGCTGGGCGCGGAGCTCCAGCCTGGAGG | |
| | | | ACGCCAGCAGCGTTTGTCAGACGGCACTCAGGAATC | |
| | | | CATCATCACCCTCCCACATCTGAATCACACCCAGGC | |
| | | | GTTCCTGTCTTGTTGTCTCAACTGGGGCAACTCCTTG | |
| | | | CAGATACTGGACCAGGTGGAGCTGAGAGCTGGCTAT | |
| | | | CCCCCGGCAATTCCTCACAACCTGTCCTGTCTGATGA | |
| | | | ACCTGACCACCTCCAGTCTGATCTGTCAGTGGGAGC | |
| | | | CCGGGCCAGAGACTCACCTTCCCACCAGCTTCACCC | |
| | | | TTAAGTCGTTCAAATCCGAGGGCAACTGCCAGACCC | |
| | | | AGGGTGATTCTATTTTGGATTGCGTGCCCAAGGACG | |
| | | | GGCAGTCTCACTGTTGTATTCCCGACAAGCACCTCCT | |
| | | | TTTATACCAGAATATGGGCATCTGGGTGCAGGCCGA | |
| | | | GAATGCCCTGGGAACGTCTATGTCTCCACAGCTATG | |
| | | | CCTGGATCCAATGGACGTAGTGAAGCTGGAGCCCCC | |
| | | | CATGCTACGCACTATGGATCCTAGCCCAGAGGCCGC | |
| | | | TCCCCCTCAGGCGGGGTGCCTGCAGCTTTGTTGGGA | |
| | | | ACCCTGGCAACCCGGGTTGCACATTAACCAGAAGTG | |
| | | | CGAGCTGCGCCACAAGCCCCAGAGAGGAGAGGCCT | |
| | | | CCTGGGCCCTCGTGGGCCCTCTGCCGTTGGAGGCCTT | |
| | | | GCAGTATGAGCTGTGCGGCCTCTTGCCCGCTACAGC | |
| | | | TTATACTCTGCAAATCAGGTGTATTCGCTGGCCTTTG | |
| | | | CCCGGACACTGGTCCGACTGGTCCCCCAGCCTTGAG | |
| | | | CTGAGAACCACGGAGCGCGCCCCCACCGTACGCTTG | |
| | | | GACACCTGGTGGCGCCAGAGGCAGCTGGACCCTCGC | |
| | | | ACTGTGCAGCTGTTCTGGAAGCCGGTGCCCCTGGAG | |
| | | | GAGGACTCCGGACGCATCCAGGGCTACGTGGTCAGC | |
| | | | TGGCGTCCGAGTGGGCAGGCCGGGGCCATCCTGCCC | |
| | | | CTGTGCAACACGACTGAGCTGAGCTGCACTTTCCAT | |
| | | | CTGCCCAGCGAGGCTCAGGAGGTGGCGCTCGTGGCT | |
| | | | TACAACTCCGCAGGCACTTCTCGCCCAACCCCCGTG | |
| | | | GTGTTCTCTGAGAGTCGGGGCCCCGCCCTGACGCGC | |
| | | | CTTCACGCCATGGCGCGCGACCCGCATTCCCTTTGG | |
| | | | GTCGGTTGGGAGCCACCTAACCCATGGCCGCAGGGC | |
| | | | TACGTGATCGAGTGGGGCCTGGGCCCCCCGAGCGCA | |
| | | | TCGAACAGCAACAAGACCTGGAGAATGGAACAGAA | |
| | | | CGGCCGCGCAACCGGCTTCCTATTAAAGGAGAACAT | |
| | | | CCGCCCGTTCCAGCTGTACGAGATCATCGTGACACC | |
| | | | GCTGTACCAGGACACTATGGGGCCTTCTCAGCACGT | |
| | | | TTACGCCTACTCCCAGGAGATGGCCCCGAGCCACGC | |
| | | | ACCGGAACTGCACCTAAAGCACATCGGTAAGACCTG | |
| | | | GGCACAGCTCGAGTGGGTCCCCGAGCCTCCCGAGCT | |
| | | | AGGCAAGTCGCCGCTGACCCATTACACCATCTTCTG | |
| | | | GACCAACGCGCAGAACCAGTCCTTTAGCGCGATCCT | |
| | | | CAATGCCTCCTCCCGCGGTTTCGTCTTGCACGGGCTG | |
| | | | GAGCCAGCCTCCTTATACCACATCCACCTGATGGCG | |
| | | | GCTTCCCAGGCTGGTGCTACCAACAGCACAGTTTTG | |
| | | | ACACTGATGACCCTGACCCCCGAGGGCTCGGAGCTC | |
| | | | CACATCATTTTGGGACTCTTCGGACTGCTGCTGCTGC | |
| | | | TGACCTGCCTGTGCGGCACTGCTTGGCTCTGCTGTTC | |
| | | | TCCCAACCGCAAGAACCCTCTTTGGCCCTCTGTCCCT | |
| | | | GACCCGGCCCATTCTAGCCTGGGATCTTGGGTCCCG | |
| | | | ACCATCATGGAAGAAGACGCGTTCCAGCTCCCTGGG | |
| | | | CTGGGCACCCCTCCGATCACGAAGCTCACGGTCCTG | |
| | | | GAGGAGGACGAGAAGAAGCCCGTACCGTGGGAGAG | |
| | | | CCACTCCTCGAATCATAGCCTAACCTCCTGCTTTACA | |
| | | | AACCAGGGGTATTTTTTTTTCCACTTGCCCGACGCTC | |
| | | | TGGAGATCGAGGCCTGCCAGGTGTACTTCACCTACG | |
| | | | ACCCCTACAGCGAGGAGGACCCTGCCGGTGATCTGC | |
| | | | CCACCCATGACGGCTATCTGCCATCAAACATTGACG | |
| | | | ACCTGCCGTCCGGAGGTCCCAGTCCTCCGTCCACCG | |
| | | | CCCCCGGCGGTTCTGGTGCCGGCGAAGAACGGATGC | |
| | | | CTCCCTCCCTGCAGGAGCGTGTGCCACGGGATTGGG | |
| | | | ACCCACAGCCCTTAGGGCCGCCTACCCCCGGCGTGC | |
| | | | CCGACCTCGTCGATTTCCAGCCGCCTCCTGAGCTGGT | |
| | | | TCTCCGAGAGGCAGGAGAGGAGGTGCCTGATGCTGG | |
| | | | ACCCCGTGAGGGCGTGAGCTTTCCTTGGTCTCGCCC | |
| | | | GCCAGGACAGGGGGAGTTCCGTGCTCTCAACGCGCG | |
| | | | TCTGCCGTTGAACACTGATGCATACCTCAGTCTTCAG | |
| | | | GAGCTGCAGGGCCAGGATCCAACCCACCTCGTTGGC | |
| | | | TCAGGGGAAGGTCGCGGCTCGCTGTTGACGTGTGGT | |
| | | | GACGTGGAGGAGAACCCCGGCCCGTACTTCGGTAAG | |
| | | | CTGGAGTCTAAACTGTCGGTGATACGGAACCTGAAC | |
| | | | GACCAGGTCCTGTTCATTGACCAGGGTAACCGCCCG | |
| | | | CTGTTCGAGGACATGACTGACTCCGATTGCCGCGAC | |
| | | | AATGCCCCCCGCACGATCTTCATTATCTCCATGTACA | |
| | | | AGGATAGCCAACCCCGTGGTATGGCCGTGACCATTA | |
| | | | GCGTCAAATGTGAGAAGATCTCTACTCTGTCCTGTG | |
| | | | AGAATAAGATTATCTCCTTCAAGGAGATGAACCCCC | |
| | | | CTGATAACATCAAAGACACTAAATCGGACATCATTT | |
| | | | TCTTTCAGAGGAGCGTGCCAGGCCACGACAACAAGA | |
| | | | TGCAGTTCGAGAGTTCTAGTTACGAGGGATATTTCCT | |
| | | | GGCCTGCGAAAAAGAACGTGACCTCTTCAAGCTAAT | |
| | | | CCTGAAGAAGGAGGACGAGCTCGGTGACAGGAGCA | |
| | | | TCATGTTTACTGTCCAAAACGAGGACTGA | |
| 5′ | | 1-21 | CCCGCCAGGATCCTGACGCGT | SEQ |
| upstream | | | | ID |
| sequence | | | | NO. |
| including | | | | 218) |
| Kozak | | | | |
| Mesothelin | n/a | 22-1482 | ATGGTGCTGCTGGTCACCAGCTTACTCCTGTGCGAG | SEQ |
| CAR | | | CTCCCCCATCCTGCGTTCTTGCTGATCCCCGACATCC | ID |
| | | | AGCAGGTGCAGCTGCAACAGTCGGGCCCCGAGTTAG | NO. |
| | | | AAAAACCCGGCGCCTCCGTGAAGATCTCGTGTAAAG | 219) |
| | | | CTTCGGGCTACTCGTTTACCGGCTACACCATGAATTG | |
| | | | GGTTAAGCAGAGCCATGGAAAGTCTCTTGAATGGAT | |
| | | | AGGCCTGATTACGCCTTATAATGGGGCCTCGAGCTA | |
| | | | CAACCAGAAATTTCGTGGTAAGGCAACTCTGACTGT | |
| | | | AGACAAGTCGTCCTCCACCGCATACATGGATCTTCT | |
| | | | GAGCCTAACCTCAGAAGACAGCGCAGTCTACTTCTG | |
| | | | CGCCCGTGGTGGTTACGATGGCCGCGGATTCGACTA | |
| | | | TTGGGGCCAGGGCACCACCGTCACCGTGAGCTCCGG | |
| | | | CGGTGGCGGGTCCGGTGGCGGCGGCTCAAGCGGAG | |
| | | | GAGGTAGTGACATCGAGTTGACCCAGTCGCCTGCCA | |
| | | | TCATGTCCGCATCCCCCGGCGAGAAGGTGACGATGA | |
| | | | CGTGTTCTGCTTCCAGCTCTGTATCGTACATGCACTG | |
| | | | GTACCAACAGAAGTCCGGCACCTCCCCCAAGCGTTG | |
| | | | GATCTACGACACTTCCAAGCTGGCGTCTGGCGTCCC | |
| | | | TGGGCGCTTTTCAGGTAGCGGGTCCGGCAATAGCTA | |
| | | | TTCCTTGACGATCAGTAGCGTGGAGGCTGAGGATGA | |
| | | | TGCCACCTACTACTGCCAGCAGTGGAGCAAGCACCC | |
| | | | GCTCACCTTCGGCGCCGGGACCAAGCTTGAAATCAA | |
| | | | GGCGGCGGCGATCGAGGTGATGTATCCTCCTCCCTA | |
| | | | CCTGGACAACGAGAAATCCAATGGCACTATCATCCA | |
| | | | CGTGAAGGGGAAGCACCTGTGCCCGAGCCCCCTGTT | |
| | | | CCCCGGCCCTTCCAAGCCTTTTTGGGTGCTTGTTGTG | |
| | | | GTCGGCGGCGTGCTGGCCTGCTACTCTCTGCTGGTA | |
| | | | ACCGTAGCGTTCATCATCTTCTGGGTGCGATCTAAGC | |
| | | | GGTCCCGGCTGTTGCACAGTGACTACATGAACATGA | |
| | | | CCCCGCGCCGCCCCGGCCCAACCCGCAAGCACTACC | |
| | | | AGCCGTACGCTCCACCTCGCGACTTTGCCGCCTACA | |
| | | | GGTCGCGAGTGAAATTTTCGCGCTCCGCGGACGCGC | |
| | | | CGGCCTATCAGCAGGGCCAGAACCAGCTTTACAACG | |
| | | | AGTTGAACCTCGGGCGCAGGGAGGAGTACGACGTG | |
| | | | CTGGATAAACGTAGAGGTCGGGACCCGGAGATGGG | |
| | | | CGGCAAACCACGCCGCAAGAACCCTCAGGAAGGCC | |
| | | | TGTACAACGAGCTCCAGAAGGACAAAATGGCTGAG | |
| | | | GCGTACTCGGAGATTGGGATGAAAGGCGAGCGGCG | |
| | | | CCGCGGCAAGGGCCACGATGGCCTGTACCAGGGGCT | |
| | | | ATCTACCGCTACAAAGGACACCTACGACGCCCTACA | |
| | | | TATGCAGGCCCTGCCACCGCGT | |
| P2A | | 1483-1548 | GGCTCTGGCGCAACCAACTTCTCGCTTCTGAAGCAG | SEQ |
| | | | GCCGGTGATGTGGAGGAAAACCCAGGTCCT | ID |
| | | | | NO. |
| | | | | 220) |
| G12/2R- | | 1549-4221 | ATGGCCCGCCTGGGCAACTGCTCGCTGACCTGGGCG | SEQ |
| 1134 ECD | | | GCCCTGATCATCCTGCTGCTGCCCGGCTCTCTGGAGG | ID |
| | | | AGTGTGGCCATATTTCCGTGTCAGCTCCAATCGTGCA | NO. |
| | | | CCTGGGCGACCCCATCACCGCTTCTTGCATCATCAA | 221) |
| | | | GCAGAACTGCTCTCACCTCGACCCTGAGCCCCAAAT | |
| | | | ACTTTGGGAGCTGGGCGCGGAGCTCCAGCCTGGAGG | |
| | | | ACGCCAGCAGCGTTTGTCAGACGGCACTCAGGAATC | |
| | | | CATCATCACCCTCCCACATCTGAATCACACCCAGGC | |
| | | | GTTCCTGTCTTGTTGTCTCAACTGGGGCAACTCCTTG | |
| | | | CAGATACTGGACCAGGTGGAGCTGAGAGCTGGCTAT | |
| | | | CCCCCGGCAATTCCTCACAACCTGTCCTGTCTGATGA | |
| | | | ACCTGACCACCTCCAGTCTGATCTGTCAGTGGGAGC | |
| | | | CCGGGCCAGAGACTCACCTTCCCACCAGCTTCACCC | |
| | | | TTAAGTCGTTCAAATCCGAGGGCAACTGCCAGACCC | |
| | | | AGGGTGATTCTATTTTGGATTGCGTGCCCAAGGACG | |
| | | | GGCAGTCTCACTGTTGTATTCCCGACAAGCACCTCCT | |
| | | | TTTATACCAGAATATGGGCATCTGGGTGCAGGCCGA | |
| | | | GAATGCCCTGGGAACGTCTATGTCTCCACAGCTATG | |
| | | | CCTGGATCCAATGGACGTAGTGAAGCTGGAGCCCCC | |
| | | | CATGCTACGCACTATGGATCCTAGCCCAGAGGCCGC | |
| | | | TCCCCCTCAGGCGGGGTGCCTGCAGCTTTGTTGGGA | |
| | | | ACCCTGGCAACCCGGGTTGCACATTAACCAGAAGTG | |
| | | | CGAGCTGCGCCACAAGCCCCAGAGAGGAGAGGCCT | |
| | | | CCTGGGCCCTCGTGGGCCCTCTGCCGTTGGAGGCCTT | |
| | | | GCAGTATGAGCTGTGCGGCCTCTTGCCCGCTACAGC | |
| | | | TTATACTCTGCAAATCAGGTGTATTCGCTGGCCTTTG | |
| | | | CCCGGACACTGGTCCGACTGGTCCCCCAGCCTTGAG | |
| | | | CTGAGAACCACGGAGCGCGCCCCCACCGTACGCTTG | |
| | | | GACACCTGGTGGCGCCAGAGGCAGCTGGACCCTCGC | |
| | | | ACTGTGCAGCTGTTCTGGAAGCCGGTGCCCCTGGAG | |
| | | | GAGGACTCCGGACGCATCCAGGGCTACGTGGTCAGC | |
| | | | TGGCGTCCGAGTGGGCAGGCCGGGGCCATCCTGCCC | |
| | | | CTGTGCAACACGACTGAGCTGAGCTGCACTTTCCAT | |
| | | | CTGCCCAGCGAGGCTCAGGAGGTGGCGCTCGTGGCT | |
| | | | TACAACTCCGCAGGCACTTCTCGCCCAACCCCCGTG | |
| | | | GTGTTCTCTGAGAGTCGGGGCCCCGCCCTGACGCGC | |
| | | | CTTCACGCCATGGCGCGCGACCCGCATTCCCTTTGG | |
| | | | GTCGGTTGGGAGCCACCTAACCCATGGCCGCAGGGC | |
| | | | TACGTGATCGAGTGGGGCCTGGGCCCCCCGAGCGCA | |
| | | | TCGAACAGCAACAAGACCTGGAGAATGGAACAGAA | |
| | | | CGGCCGCGCAACCGGCTTCCTATTAAAGGAGAACAT | |
| | | | CCGCCCGTTCCAGCTGTACGAGATCATCGTGACACC | |
| | | | GCTGTACCAGGACACTATGGGGCCTTCTCAGCACGT | |
| | | | TTACGCCTACTCCCAGGAGATGGCCCCGAGCCACGC | |
| | | | ACCGGAACTGCACCTAAAGCACATCGGTAAGACCTG | |
| | | | GGCACAGCTCGAGTGGGTCCCCGAGCCTCCCGAGCT | |
| | | | AGGCAAGTCGCCGCTGACCCATTACACCATCTTCTG | |
| | | | GACCAACGCGCAGAACCAGTCCTTTAGCGCGATCCT | |
| | | | CAATGCCTCCTCCCGCGGTTTCGTCTTGCACGGGCTG | |
| | | | GAGCCAGCCTCCTTATACCACATCCACCTGATGGCG | |
| | | | GCTTCCCAGGCTGGTGCTACCAACAGCACAGTTTTG | |
| | | | ACACTGATGACCCTGACCCCCGAGGGCTCGGAGCTC | |
| | | | CACATCATTTTGGGACTCTTCGGACTGCTGCTGCTGC | |
| | | | TGACCTGCCTGTGCGGCACTGCTTGGCTCTGCTGTTC | |
| | | | TCCCAACCGCAAGAACCCTCTTTGGCCCTCTGTCCCT | |
| | | | GACCCGGCCCATTCTAGCCTGGGATCTTGGGTCCCG | |
| | | | ACCATCATGGAAGAAGACGCGTTCCAGCTCCCTGGG | |
| | | | CTGGGCACCCCTCCGATCACGAAGCTCACGGTCCTG | |
| | | | GAGGAGGACGAGAAGAAGCCCGTACCGTGGGAGAG | |
| | | | CCACTCCTCGAATCATAGCCTAACCTCCTGCTTTACA | |
| | | | AACCAGGGGTATTTTTTTTTCCACTTGCCCGACGCTC | |
| | | | TGGAGATCGAGGCCTGCCAGGTGTACTTCACCTACG | |
| | | | ACCCCTACAGCGAGGAGGACCCTGCCGGTGATCTGC | |
| | | | CCACCCATGACGGCTATCTGCCATCAAACATTGACG | |
| | | | ACCTGCCGTCCGGAGGTCCCAGTCCTCCGTCCACCG | |
| | | | CCCCCGGCGGTTCTGGTGCCGGCGAAGAACGGATGC | |
| | | | CTCCCTCCCTGCAGGAGCGTGTGCCACGGGATTGGG | |
| | | | ACCCACAGCCCTTAGGGCCGCCTACCCCCGGCGTGC | |
| | | | CCGACCTCGTCGATTTCCAGCCGCCTCCTGAGCTGGT | |
| | | | TCTCCGAGAGGCAGGAGAGGAGGTGCCTGATGCTGG | |
| | | | ACCCCGTGAGGGCGTGAGCTTTCCTTGGTCTCGCCC | |
| | | | GCCAGGACAGGGGGAGTTCCGTGCTCTCAACGCGCG | |
| | | | TCTGCCGTTGAACACTGATGCATACCTCAGTCTTCAG | |
| | | | GAGCTGCAGGGCCAGGATCCAACCCACCTCGTT | |
| T2A | | 4222-4284 | GGCTCAGGGGAAGGTCGCGGCTCGCTGTTGACGTGT | SEQ |
| | | | GGTGACGTGGAGGAGAACCCCGGCCCG | ID |
| | | | | NO. |
| | | | | 222) |
| IL-18 | NM_001562.4 | 4285-4758 | TACTTCGGTAAGCTGGAGTCTAAACTGTCGGTGATA | SEQ |
| | | | CGGAACCTGAACGACCAGGTCCTGTTCATTGACCAG | ID |
| | | | GGTAACCGCCCGCTGTTCGAGGACATGACTGACTCC | NO. |
| | | | GATTGCCGCGACAATGCCCCCCGCACGATCTTCATT | 223) |
| | | | ATCTCCATGTACAAGGATAGCCAACCCCGTGGTATG | |
| | | | GCCGTGACCATTAGCGTCAAATGTGAGAAGATCTCT | |
| | | | ACTCTGTCCTGTGAGAATAAGATTATCTCCTTCAAGG | |
| | | | AGATGAACCCCCCTGATAACATCAAAGACACTAAAT | |
| | | | CGGACATCATTTTCTTTCAGAGGAGCGTGCCAGGCC | |
| | | | ACGACAACAAGATGCAGTTCGAGAGTTCTAGTTACG | |
| | | | AGGGATATTTCCTGGCCTGCGAAAAAGAACGTGACC | |
| | | | TCTTCAAGCTAATCCTGAAGAAGGAGGACGAGCTCG | |
| | | | GTGACAGGAGCATCATGTTTACTGTCCAAAACGAGG | |
| | | | ACTGA | |
| G12- | | 1-3210 | ATGGCTCGCCTCGGAAATTGTTCCCTGACCTGGGCA | SEQ |
| 2R1_134_ | | | GCATTGATCATTCTCCTGCTACCAGGGAGTCTCGAA | ID |
| ECD_T2A_ | | | GAATGTGGCCATATCTCTGTAAGCGCCCCTATTGTGC | NO. |
| hIL-18 | | | ACTTGGGAGACCCAATCACAGCATCATGCATCATCA | 224) |
| | | | AGCAGAATTGCTCTCATCTCGATCCAGAGCCACAGA | |
| | | | TACTCTGGGAGCTGGGCGCAGAACTGCAACCCGGAG | |
| | | | GGCGCCAGCAGAGACTGAGTGATGGAACACAGGAG | |
| | | | AGCATTATCACTTTACCTCATCTGAACCACACCCAG | |
| | | | GCCTTTCTTAGCTGCTGTTTGAACTGGGGCAACAGTC | |
| | | | TCCAGATTCTGGACCAGGTGGAGCTGAGGGCGGGTT | |
| | | | ACCCGCCCGCCATTCCACACAATTTGTCCTGTCTCAT | |
| | | | GAACCTGACAACAAGTAGCCTGATATGCCAGTGGGA | |
| | | | GCCAGGGCCGGAGACCCATCTCCCAACTAGTTTCAC | |
| | | | GCTCAAGAGCTTCAAGTCAGAGGGGAACTGCCAGAC | |
| | | | ACAAGGGGACAGCATTCTCGACTGTGTGCCCAAGGA | |
| | | | CGGGCAGAGTCACTGTTGCATTCCAGACAAGCATCT | |
| | | | GCTGCTGTATCAGAACATGGGCATTTGGGTTCAGGC | |
| | | | CGAAAATGCCCTCGGTACTTCCATGAGTCCACAGTT | |
| | | | GTGTCTTGACCCCATGGATGTGGTGAAGCTGGAACC | |
| | | | GCCAATGCTGAGGACCATGGATCCCTCACCTGAGGC | |
| | | | TGCTCCGCCACAGGCAGGGTGTCTGCAGCTGTGCTG | |
| | | | GGAGCCCTGGCAGCCTGGATTGCACATAAACCAAAA | |
| | | | ATGTGAGCTGAGGCACAAGCCCCAGAGAGGTGAGG | |
| | | | CGAGTTGGGCCCTGGTAGGGCCTCTTCCTCTAGAAG | |
| | | | CCCTGCAGTATGAATTGTGTGGATTACTTCCCGCAAC | |
| | | | CGCCTACACTCTTCAGATCAGGTGTATCCGGTGGCCT | |
| | | | CTGCCCGGCCACTGGAGCGACTGGTCTCCGAGCCTC | |
| | | | GAATTGAGAACAACAGAACGGGCTCCTACGGTGCGT | |
| | | | CTGGATACATGGTGGAGGCAGCGACAGTTGGACCCG | |
| | | | CGTACAGTGCAGCTGTTCTGGAAGCCCGTACCTCTG | |
| | | | GAAGAGGATAGTGGTCGGATTCAGGGATATGTGGTC | |
| | | | TCTTGGCGGCCTTCCGGTCAGGCTGGGGCTATCCTTC | |
| | | | CCTTATGCAACACTACAGAGCTCAGCTGTACCTTTCA | |
| | | | TTTACCCAGCGAAGCCCAGGAAGTGGCTCTCGTCGC | |
| | | | CTATAATTCCGCTGGAACGTCAAGACCCACCCCTGT | |
| | | | CGTGTTCTCTGAGTCTCGGGGACCTGCCCTAACCAG | |
| | | | GCTCCATGCAATGGCACGCGATCCTCACAGTCTGTG | |
| | | | GGTTGGCTGGGAACCACCAAATCCCTGGCCTCAGGG | |
| | | | CTATGTCATCGAATGGGGCCTTGGCCCCCCATCAGC | |
| | | | CTCCAATAGCAACAAGACCTGGAGAATGGAGCAAA | |
| | | | ATGGGAGAGCAACCGGGTTCCTGCTGAAGGAGAAC | |
| | | | ATCCGACCGTTCCAGCTTTATGAAATTATTGTAACTC | |
| | | | CACTGTACCAGGACACAATGGGACCATCTCAGCATG | |
| | | | TCTACGCTTATTCCCAGGAGATGGCGCCTAGTCATG | |
| | | | CTCCTGAATTACACTTGAAGCACATAGGGAAAACTT | |
| | | | GGGCCCAATTAGAGTGGGTCCCGGAGCCTCCCGAGC | |
| | | | TGGGGAAAAGCCCATTGACCCATTACACGATATTTT | |
| | | | GGACCAATGCTCAGAACCAATCCTTTTCTGCCATCCT | |
| | | | GAATGCCTCATCTCGAGGCTTTGTGCTGCATGGCCTA | |
| | | | GAGCCTGCATCACTCTACCACATTCACCTGATGGCC | |
| | | | GCCAGCCAGGCGGGTGCCACAAACTCCACAGTTCTC | |
| | | | ACTTTAATGACTCTAACCCCAGAGGGCAGTGAACTG | |
| | | | CACATCATATTAGGGTTATTCGGATTGCTGCTTCTTT | |
| | | | TGACTTGCCTCTGTGGCACTGCGTGGCTGTGCTGCTC | |
| | | | CCCGAACCGAAAGAATCCTCTCTGGCCTAGCGTTCC | |
| | | | CGATCCTGCTCACAGTTCACTAGGATCATGGGTGCC | |
| | | | AACCATCATGGAAGAGGATGCCTTCCAGCTACCAGG | |
| | | | ACTGGGTACGCCGCCCATAACCAAATTAACGGTCCT | |
| | | | TGAAGAGGATGAGAAGAAACCAGTTCCATGGGAAA | |
| | | | GCCACTCTTCCAATCATAGCCTCACCTCTTGCTTCAC | |
| | | | CAACCAAGGGTATTTTTTTTTCCACCTGCCCGACGCT | |
| | | | CTGGAAATTGAGGCCTGCCAAGTGTATTTCACCTAC | |
| | | | GACCCTTACTCTGAAGAAGATCCGGCAGGCGACCTA | |
| | | | CCCACTCATGATGGCTACCTGCCGTCAAACATCGAC | |
| | | | GACTTACCGAGTGGAGGACCCTCCCCACCTTCTACA | |
| | | | GCCCCTGGTGGCTCCGGTGCAGGGGAGGAAAGGAT | |
| | | | GCCTCCCTCACTGCAGGAGAGAGTCCCCAGAGACTG | |
| | | | GGATCCTCAGCCACTTGGACCACCCACACCTGGTGT | |
| | | | GCCTGACCTTGTGGATTTTCAGCCCCCTCCTGAGCTT | |
| | | | GTTCTTCGAGAAGCAGGTGAGGAGGTTCCTGATGCA | |
| | | | GGCCCACGTGAGGGGGTCTCCTTTCCCTGGTCCAGG | |
| | | | CCTCCTGGCCAAGGAGAGTTCCGCGCTCTGAATGCA | |
| | | | CGCTTGCCTCTGAACACAGATGCTTACCTGAGCCTA | |
| | | | CAAGAGTTACAAGGCCAGGATCCGACACACCTGGTG | |
| | | | GGATCTGGCGAGGGTAGAGGCAGCCTCCTAACTTGT | |
| | | | GGCGACGTGGAGGAGAACCCCGGTCCCTACTTCGGT | |
| | | | AAGCTGGAGTCTAAACTGTCGGTGATACGGAACCTG | |
| | | | AACGACCAGGTCCTGTTCATTGACCAGGGTAACCGC | |
| | | | CCGCTGTTCGAGGACATGACTGACTCCGATTGCCGC | |
| | | | GACAATGCCCCCCGCACGATCTTCATTATCTCCATGT | |
| | | | ACAAGGATAGCCAACCCCGTGGTATGGCCGTGACCA | |
| | | | TTAGCGTCAAATGTGAGAAGATCTCTACTCTGTCCTG | |
| | | | TGAGAATAAGATTATCTCCTTCAAGGAGATGAACCC | |
| | | | CCCTGATAACATCAAAGACACTAAATCGGACATCAT | |
| | | | TTTCTTTCAGAGGAGCGTGCCAGGCCACGACAACAA | |
| | | | GATGCAGTTCGAGAGTTCTAGTTACGAGGGATATTT | |
| | | | CCTGGCCTGCGAAAAAGAACGTGACCTCTTCAAGCT | |
| | | | AATCCTGAAGAAGGAGGACGAGCTCGGTGACAGGA | |
| | | | GCATCATGTTTACTGTCCAAAACGAGGACTGATGA | |
| G12/2R- | | 1-2673 | ATGGCTCGCCTCGGAAATTGTTCCCTGACCTGGGCA | SEQ |
| 1134 ECD | | | GCATTGATCATTCTCCTGCTACCAGGGAGTCTCGAA | ID |
| | | | GAATGTGGCCATATCTCTGTAAGCGCCCCTATTGTGC | NO. |
| | | | ACTTGGGAGACCCAATCACAGCATCATGCATCATCA | 225) |
| | | | AGCAGAATTGCTCTCATCTCGATCCAGAGCCACAGA | |
| | | | TACTCTGGGAGCTGGGCGCAGAACTGCAACCCGGAG | |
| | | | GGCGCCAGCAGAGACTGAGTGATGGAACACAGGAG | |
| | | | AGCATTATCACTTTACCTCATCTGAACCACACCCAG | |
| | | | GCCTTTCTTAGCTGCTGTTTGAACTGGGGCAACAGTC | |
| | | | TCCAGATTCTGGACCAGGTGGAGCTGAGGGCGGGTT | |
| | | | ACCCGCCCGCCATTCCACACAATTTGTCCTGTCTCAT | |
| | | | GAACCTGACAACAAGTAGCCTGATATGCCAGTGGGA | |
| | | | GCCAGGGCCGGAGACCCATCTCCCAACTAGTTTCAC | |
| | | | GCTCAAGAGCTTCAAGTCAGAGGGGAACTGCCAGAC | |
| | | | ACAAGGGGACAGCATTCTCGACTGTGTGCCCAAGGA | |
| | | | CGGGCAGAGTCACTGTTGCATTCCAGACAAGCATCT | |
| | | | GCTGCTGTATCAGAACATGGGCATTTGGGTTCAGGC | |
| | | | CGAAAATGCCCTCGGTACTTCCATGAGTCCACAGTT | |
| | | | GTGTCTTGACCCCATGGATGTGGTGAAGCTGGAACC | |
| | | | GCCAATGCTGAGGACCATGGATCCCTCACCTGAGGC | |
| | | | TGCTCCGCCACAGGCAGGGTGTCTGCAGCTGTGCTG | |
| | | | GGAGCCCTGGCAGCCTGGATTGCACATAAACCAAAA | |
| | | | ATGTGAGCTGAGGCACAAGCCCCAGAGAGGTGAGG | |
| | | | CGAGTTGGGCCCTGGTAGGGCCTCTTCCTCTAGAAG | |
| | | | CCCTGCAGTATGAATTGTGTGGATTACTTCCCGCAAC | |
| | | | CGCCTACACTCTTCAGATCAGGTGTATCCGGTGGCCT | |
| | | | CTGCCCGGCCACTGGAGCGACTGGTCTCCGAGCCTC | |
| | | | GAATTGAGAACAACAGAACGGGCTCCTACGGTGCGT | |
| | | | CTGGATACATGGTGGAGGCAGCGACAGTTGGACCCG | |
| | | | CGTACAGTGCAGCTGTTCTGGAAGCCCGTACCTCTG | |
| | | | GAAGAGGATAGTGGTCGGATTCAGGGATATGTGGTC | |
| | | | TCTTGGCGGCCTTCCGGTCAGGCTGGGGCTATCCTTC | |
| | | | CCTTATGCAACACTACAGAGCTCAGCTGTACCTTTCA | |
| | | | TTTACCCAGCGAAGCCCAGGAAGTGGCTCTCGTCGC | |
| | | | CTATAATTCCGCTGGAACGTCAAGACCCACCCCTGT | |
| | | | CGTGTTCTCTGAGTCTCGGGGACCTGCCCTAACCAG | |
| | | | GCTCCATGCAATGGCACGCGATCCTCACAGTCTGTG | |
| | | | GGTTGGCTGGGAACCACCAAATCCCTGGCCTCAGGG | |
| | | | CTATGTCATCGAATGGGGCCTTGGCCCCCCATCAGC | |
| | | | CTCCAATAGCAACAAGACCTGGAGAATGGAGCAAA | |
| | | | ATGGGAGAGCAACCGGGTTCCTGCTGAAGGAGAAC | |
| | | | ATCCGACCGTTCCAGCTTTATGAAATTATTGTAACTC | |
| | | | CACTGTACCAGGACACAATGGGACCATCTCAGCATG | |
| | | | TCTACGCTTATTCCCAGGAGATGGCGCCTAGTCATG | |
| | | | CTCCTGAATTACACTTGAAGCACATAGGGAAAACTT | |
| | | | GGGCCCAATTAGAGTGGGTCCCGGAGCCTCCCGAGC | |
| | | | TGGGGAAAAGCCCATTGACCCATTACACGATATTTT | |
| | | | GGACCAATGCTCAGAACCAATCCTTTTCTGCCATCCT | |
| | | | GAATGCCTCATCTCGAGGCTTTGTGCTGCATGGCCTA | |
| | | | GAGCCTGCATCACTCTACCACATTCACCTGATGGCC | |
| | | | GCCAGCCAGGCGGGTGCCACAAACTCCACAGTTCTC | |
| | | | ACTTTAATGACTCTAACCCCAGAGGGCAGTGAACTG | |
| | | | CACATCATATTAGGGTTATTCGGATTGCTGCTTCTTT | |
| | | | TGACTTGCCTCTGTGGCACTGCGTGGCTGTGCTGCTC | |
| | | | CCCGAACCGAAAGAATCCTCTCTGGCCTAGCGTTCC | |
| | | | CGATCCTGCTCACAGTTCACTAGGATCATGGGTGCC | |
| | | | AACCATCATGGAAGAGGATGCCTTCCAGCTACCAGG | |
| | | | ACTGGGTACGCCGCCCATAACCAAATTAACGGTCCT | |
| | | | TGAAGAGGATGAGAAGAAACCAGTTCCATGGGAAA | |
| | | | GCCACTCTTCCAATCATAGCCTCACCTCTTGCTTCAC | |
| | | | CAACCAAGGGTATTTTTTTTTCCACCTGCCCGACGCT | |
| | | | CTGGAAATTGAGGCCTGCCAAGTGTATTTCACCTAC | |
| | | | GACCCTTACTCTGAAGAAGATCCGGCAGGCGACCTA | |
| | | | CCCACTCATGATGGCTACCTGCCGTCAAACATCGAC | |
| | | | GACTTACCGAGTGGAGGACCCTCCCCACCTTCTACA | |
| | | | GCCCCTGGTGGCTCCGGTGCAGGGGAGGAAAGGAT | |
| | | | GCCTCCCTCACTGCAGGAGAGAGTCCCCAGAGACTG | |
| | | | GGATCCTCAGCCACTTGGACCACCCACACCTGGTGT | |
| | | | GCCTGACCTTGTGGATTTTCAGCCCCCTCCTGAGCTT | |
| | | | GTTCTTCGAGAAGCAGGTGAGGAGGTTCCTGATGCA | |
| | | | GGCCCACGTGAGGGGGTCTCCTTTCCCTGGTCCAGG | |
| | | | CCTCCTGGCCAAGGAGAGTTCCGCGCTCTGAATGCA | |
| | | | CGCTTGCCTCTGAACACAGATGCTTACCTGAGCCTA | |
| | | | CAAGAGTTACAAGGCCAGGATCCGACACACCTGGTG | |
| T2A | | 2674-2736 | GGATCTGGCGAGGGTAGAGGCAGCCTCCTAACTTGT | SEQ |
| | | | GGCGACGTGGAGGAGAACCCCGGTCCC | ID |
| | | | | NO. |
| | | | | 226) |

TABLE 32
PROTEIN SEQUENCE OF HUMAN IL-7RA EXTRACELLULAR,
TRANSMEMBRANE AND INTRACELLULAR DOMAINS AND SIGNAL PEPTIDE

| | | Domain & | | SEQ |
|---|---|---|---|---|
| | | residue | | ID |
| Protein | Uniprot ID | numbering | Polypeptide Sequence | NO. |
| IL-7Rα | P16871 | ECD: 1-219 | ESGYAQNGDLEDAELDDYSFSCYSQLEVNGSQHSLTCAFEDPDV | 187 |
| | | | NITNLEFEICGALVEVKCLNFRKLQEIYFIETKKFLLIGKSNICVKVG | |
| | | | EKSLTCKKIDLTTIVKPEAPFDLSVVYREGANDFVVTFNTSHLQKK | |
| | | | YVKVLMHDVAYRQEKDENKWTHVNLSSTKLTLLQRKLQPAAMY | |
| | | | EIKVRSIPDHYFKGFWSEWSPSYYFRTPEINNSSGEMD | |
| IL-7Rα | P16871 | TMD: 220-244 | PILLTISILSFFSVALLVILACVLW | 188 |
| IL-7Rα | P16871 | ICD: 245-439 | KKRIKPIVWPSLPDHKKTLEHLCKKPRKNLNVSFNPESFLDCQIHR | 189 |
| | | | VDDIQARDEVEGFLQDTFPQQLEESEKQRLGGDVQSPNCPSED | |
| | | | VVITPESFGRDSSLTCLAGNVSACDAPILSSSRSLDCRESGKNGPH | |
| | | | VYQDLLLSLGTTNSTLPPPFSLQSGILTLNPVAQGQPILTSLGSNQ | |
| | | | EEAYVTMSSFYQNQ | |
| IL-7Rα | P16871 | Signal peptide | MTILGTTFGMVFSLLQVVSG | 190 |
| IL-7 | A8K673 | Cytokine: 1-152 | DCDIEGKDGKQYESVLMVSIDQLLDSMKEIGSNCLNNEFNFFKRHI | 191 |
| | | | CDANKEGMFLFRAARKLRQFLKMNSTGDFDLHLLKVSEGTTILLNC | |
| | | | TGQVKGRKPAALGEAQPTKSLEENKSLKEQKKLNDLCFLKRLLQEIK | |
| | | | TCWNKILMGTKEH | |

TABLE 33
INTRACELLULAR DOMAINS (ICDS). THE ICD CONSISTS OF ONE
OF THE FOLLOWING:

| | | | | SEQ |
|---|---|---|---|---|
| Engineered | | | | ID |
| Construct | Protein (Domain) | Uniprot ID | Polypeptide Sequence | NO. |
| 7/2R-1 | IL-2Rβ: | P14784 | NCRNTGPWLKKVLKCNTPDPSKFFSQLSSEHGGDVQKWLSSPFP | 192 |
| | Box 1 underlined | | SSSFSPGGLAPEISPLEVLERDKVTQLLLQQDKVPEPASLSSNHSLT | |
| | Box 2, Shc, & STAT5 | | SCFTNQGYFFFHLPDALEIEACQVYFTYDPYSEEDPDEGVAGAPT | |
| | binding sites bold | | GSSPQPLQPLSGEDDAYCTFPSRDDLLLFSPSLLGGPSPPSTAPGG | |
| | underlined | | SGAGEERMPPSLQERVPRDWDPQPLGPPTPGVPDLVDFQPPPE | |
| | | | LVLREAGEEVPDAGPREGVSFPWSRPPGQGEFRALNARLPLNTD | |
| | | | AYLSLQELQGQDPTHLV* | |
| 7/2R-2 | IL-7Rα: | P16871 | KKRIKPIVWPSLPDHKKTLEHLCKKPRKNLNVSFNPESFLDCQIHR | 193 |
| | Box 1 underlined | | VDDIQARDEVEG | |
| | Box 2 bold | | | |
| | underlined | | | |
| | IL-2Rβ: | P14784 | LERDKVTQLLLQQDKVPEPASLSSNHSLTSCFTNQGYFFFHLPDA | 194 |
| | Shc & STAT5 binding | | LEIEACQVYFTYDPYSEEDPDEGVAGAPTGSSPQPLQPLSGEDDA | |
| | sites bold underlined | | YCTFPSRDDLLLFSPSLLGGPSPPSTAPGGSGAGEERMPPSLQER | |
| | *stop codon | | VPRDWDPQPLGPPTPGVPDLVDFQPPPELVLREAGEEVPDAGP | |
| | | | REGVSFPWSRPPGQGEFRALNARLPLNTDAYLSLQELQGQDPT | |
| | | | HLV* | |
| 7/2/12R-1 | IL-2Rβ: | P14784 | NCRNTGPWLKKVLKCNTPDPSKFFSQLSSEHGGDVQKWLSSPFP | 192 |
| | Box 1 underlined | | SSSFSPGGLAPEISPLEVLERDKVTQLLLQQDKVPEPASLSSNHSLT | |
| | Box 2, Shc, & STAT5 | | SCFTNQGYFFFHLPDALEIEACQVYFTYDPYSEEDPDEGVAGAPT | |
| | binding sites bold | | GSSPQPLQPLSGEDDAYCTFPSRDDLLLFSPSLLGGPSPPSTAPGG | |
| | underlined | | SGAGEERMPPSLQERVPRDWDPQPLGPPTPGVPDLVDFQPPPE | |
| | | | LVLREAGEEVPDAGPREGVSFPWSRPPGQGEFRALNARLPLNTD | |
| | | | AYLSLQELQGQDPTHLV | |
| | IL-12Rβ2: | Q99665 | TVLPAGDLPTHDGYLPSNIDDLPSHEAPLADSLEELEPQHISLSVF | 195 |
| | STAT4 binding site | | PSSSLHPLTFSCGDKLTLDQLKMRCDSLML* | |
| | bold underlined | | | |
| 7/2/12R-2 | IL-7Rα: | P16871 | KKRIKPIVWPSLPDHKKTLEHLCKKPRKNLNVSFNPESFLDCQIHR | 193 |
| | Box 1 underlined | | VDDIQARDEVEG | |
| | Box 2 bold | | | |
| | underlined | | | |
| | IL-2Rβ: | P14784 | LERDKVTQLLLQQDKVPEPASLSSNHSLTSCFTNQGYFFFHLPDA | 194 |
| | Shc & STAT5 binding | | LEIEACQVYFTYDPYSEEDPDEGVAGAPTGSSPQPLQPLSGEDDA | |
| | sites bold underlined | | YCTFPSRDDLLLFSPSLLGGPSPPSTAPGGSGAGEERMPPSLQER | |
| | *stop codon | | VPRDWDPQPLGPPTPGVPDLVDFQPPPELVLREAGEEVPDAGP | |
| | | | REGVSFPWSRPPGQGEFRALNARLPLNTDAYLSLQELQGQDPT | |
| | | | HLV | |
| | IL-12Rβ2: | Q99665 | TVLPAGDLPTHDGYLPSNIDDLPSHEAPLADSLEELEPQHISLSVF | 195 |
| | STAT4 binding site | | PSSSLHPLTFSCGDKLTLDQLKMRCDSLML* | |
| | bold underlined | | | |
| 7/12/2R-1 | IL-2Rβ: | P14784 | NCRNTGPWLKKVLKCNTPDPSKFFSQLSSEHGGDVQKWLSSPFP | 196 |
| | Box 1 underlined | | SSSFSPGGLAPEISPLEVLERDKVTQLLLQQDKVPEPASLSSNHSLT | |
| | Box 2 and Shc | | SCFTNQGYFFFHLPDALEIEACQVYFTYDPYSEEDP | |
| | binding sites bold | | | |
| | underlined | | | |
| | IL-12Rβ2: | Q99665 | AGDLPTHDGYLPSNIDDLPS | 197 |
| | STAT4 binding site | | | |
| | bold underlined | | | |
| | IL-2Rβ: | P14784 | GGPSPPSTAPGGSGAGEERMPPSLQERVPRDWDPQPLGPPTP | 198 |
| | STAT5 binding site | | GVPDLVDFQPPPELVLREAGEEVPDAGPREGVSFPWSRPPGQG | |
| | bold underlined | | EFRALNARLPLNTDAYLSLQELQGQDPTHLV* | |
| | *stop codon | | | |
| 7/12/2R-2 | IL-7Rα: | P16871 | KKRIKPIVWPSLPDHKKTLEHLCKKPRKNLNVSFNPESFLDCQIHR | 199 |
| | Box 1 underlined | | VDDIQAR | |
| | Box 2 bold | | | |
| | underlined | | | |
| | IL-12Rβ2: | Q99665 | AGDLPTHDGYLPSNIDDLPSHEAPLADSLEELEPQHISLSVFPSSSL | 200 |
| | STAT4 binding site | | HPLTFSCGDKLTLDQLKMRC | |
| | bold underlined | | | |
| | IL-2Rβ: | P14784 | LERDKVTQLLLQQDKVPEPASLSSNHSLTSCFTNQGYFFFHLPDA | 194 |
| | Shc & STAT5 binding | | LEIEACQVYFTYDPYSEEDPDEGVAGAPTGSSPQPLQPLSGEDDA | |
| | sites bold underlined | | YCTFPSRDDLLLFSPSLLGGPSPPSTAPGGSGAGEERMPPSLQER | |
| | *stop codon | | VPRDWDPQPLGPPTPGVPDLVDFQPPPELVLREAGEEVPDAGP | |
| | | | REGVSFPWSRPPGQGEFRALNARLPLNTDAYLSLQELQGQDPT | |
| | | | HLV* | |
| 7/21-R1 | IL-21R: | Q9HBE5 | LWRLWKKIWAVPSPERFFMPLYKGCSGDFKKWVGAPFTGSSLE | 201 |
| | Box 1 underlined | | LGPWSPEVPSTLEVYSCHPPRSPAKRLQLTELQEPAELVESDGVP | |
| | Box 2 & STAT3 | | KPSFWPTAQNSGGSAYSEERDRPYGLVSIDTVTVLDAEGPCTWP | |
| | binding site bold | | CSCEDDGYPALDLDAGLEPSPGLEDPLLDAGTTVLSCGCVSAGSP | |
| | underlined | | GLGGPLGSLLDRLKPPLADGEDWAGGLPWGGRSPGGVSESEAG | |
| | | | SPLAGLDMDTFDSGFVGSDCSSPVECDFTSPGDEGPPRSYLRQ | |
| | | | WVVIPPPLSSPGPQAS* | |
| 7/21-R2 | IL-7Rα: | P16871 | KKRIKPIVWPSLPDHKKTLEHLCKKPRKNLNVSFNPESFLDCQIHR | 202 |
| | Box 1 underlined | | VDDIQARD | |
| | Box 2 bold | | | |
| | underlined | | | |
| | IL-21R: | Q9HBE5 | RSPAKRLQLTELQEPAELVESDGVPKPSFWPTAQNSGGSAYSEE | 203 |
| | STAT3 binding site | | RDRPYGLVSIDTVTVLDAEGPCTWPCSCEDDGYPALDLDAGLEP | |
| | bold underlined | | SPGLEDPLLDAGTTVLSCGCVSAGSPGLGGPLGSLLDRLKPPLAD | |
| | | | GEDWAGGLPWGGRSPGGVSESEAGSPLAGLDMDTFDSGFVGS | |
| | | | DCSSPVECDFTSPGDEGPPRSYLRQWVVIPPPLSSPGPQAS* | |
| 7/21/7R-1 | IL-21R: | Q9HBE5 | LWRLWKKIWAVPSPERFFMPLYKGCSGDFKKWVGAPFTGSSLE | 201 |
| | Box 1 underlined | | LGPWSPEVPSTLEVYSCHPPRSPAKRLQLTELQEPAELVESDGVP | |
| | Box 2 & STAT3 | | KPSFWPTAQNSGGSAYSEERDRPYGLVSIDTVTVLDAEGPCTWP | |
| | binding site bold | | CSCEDDGYPALDLDAGLEPSPGLEDPLLDAGTTVLSCGCVSAGSP | |
| | underlined | | GLGGPLGSLLDRLKPPLADGEDWAGGLPWGGRSPGGVSESEAG | |
| | | | SPLAGLDMDTFDSGFVGSDCSSPVECDFTSPGDEGPPRSYLRQ | |
| | | | WVVIPPPLSSPGPQAS | |
| | IL-7Rα: | P16871 | DCRESGKNGPHVYQDLLLSLGTTNSTLPPPFSLQSGILTLNPVAQ | 204 |
| | STAT5 and PI3K | | GQPILTSLGSNQEEAYVTMSSFYQNQ* | |
| | binding sites | | | |
| | boldunderlined | | | |
| 7/7/21R-1 | IL-7Rα: Box 1 | P16871 | KKRIKPIVWPSLPDHKKTLEHLCKKPRKNLNVSFNPESFLDCQIHR | 205 |
| | underlined | | VDDIQARDEVEGFLQDTFPQQLEESEKQRLGGDVQSPNCPSED | |
| | Box 2, STAT5 & PI3K | | VVITPESFGRDSSLTCLAGNVSACDAPILSSSRSLDCRESGKNGPH | |
| | binding sites bold | | VYQDLLLSLGTTNSTLPPPFSLQSGILTLNPVAQGQPILTSLGSNQ | |
| | underlined | | EEAYVTMSSFYQNQ | |
| | IL-21R: | Q9HBE5 | SPGDEGPPRSYLRQWVVIPPPLSSPGPQAS* | 206 |
| | STAT3 binding site | | | |
| | bold underlined | | | |
| 7/2/21R-1 | IL-2Rβ: | P14784 | NCRNTGPWLKKVLKCNTPDPSKFFSQLSSEHGGDVQKWLSSPFP | 192 |
| | Box 1 underlined | | SSSFSPGGLAPEISPLEVLERDKVTQLLLQQDKVPEPASLSSNHSLT | |
| | Box 2, Shc & STAT5 | | SCFTNQGYFFFHLPDALEIEACQVYFTYDPYSEEDPDEGVAGAPT | |
| | binding sites bold | | GSSPQPLQPLSGEDDAYCTFPSRDDLLLFSPSLLGGPSPPSTAPGG | |
| | underlined | | SGAGEERMPPSLQERVPRDWDPQPLGPPTPGVPDLVDFQPPPE | |
| | | | LVLREAGEEVPDAGPREGVSFPWSRPPGQGEFRALNARLPLNTD | |
| | | | AYLSLQELQGQDPTHLV | |
| | IL-21R: | Q9HBE5 | SPGDEGPPRSYLRQWVVIPPPLSSPGPQAS* | 206 |
| | STAT3 binding site | | | |
| | bold underlined | | | |
| 7/2/21R-2 | IL-7Rα: | P16871 | KKRIKPIVWPSLPDHKKTLEHLCKKPRKNLNVSFNPESFLDCQIHR | 193 |
| | Box 1 underlined | | VDDIQARDEVEG | |
| | Box 2 bold | | | |
| | underlined | | | |
| | IL-2Rβ: | P14784 | LERDKVTQLLLQQDKVPEPASLSSNHSLTSCFTNQGYFFFHLPDA | 194 |
| | Shc & STAT5 binding | | LEIEACQVYFTYDPYSEEDPDEGVAGAPTGSSPQPLQPLSGEDDA | |
| | sites bold underlined | | YCTFPSRDDLLLFSPSLLGGPSPPSTAPGGSGAGEERMPPSLQER | |
| | *stop codon | | VPRDWDPQPLGPPTPGVPDLVDFQPPPELVLREAGEEVPDAGP | |
| | | | REGVSFPWSRPPGQGEFRALNARLPLNTDAYLSLQELQGQDPT | |
| | | | HLV | |
| | IL-21R: | Q9HBE5 | SPGDEGPPRSYLRQWVVIPPPLSSPGPQAS* | 206 |
| | STAT3 binding site | | | |
| | bold underlined | | | |
| 7/2/12/ | IL-2Rβ: | P14784 | NCRNTGPWLKKVLKCNTPDPSKFFSQLSSEHGGDVQKWLSSPFP | 192 |
| 21R-1 | Box 1 underlined | | SSSFSPGGLAPEISPLEVLERDKVTQLLLQQDKVPEPASLSSNHSLT | |
| | Box 2, Shc & STAT5 | | SCFTNQGYFFFHLPDALEIEACQVYFTYDPYSEEDPDEGVAGAPT | |
| | binding sites bold | | GSSPQPLQPLSGEDDAYCTFPSRDDLLLFSPSLLGGPSPPSTAPGG | |
| | underlined | | SGAGEERMPPSLQERVPRDWDPQPLGPPTPGVPDLVDFQPPPE | |
| | | | LVLREAGEEVPDAGPREGVSFPWSRPPGQGEFRALNARLPLNTD | |
| | | | AYLSLQELQGQDPTHLV | |
| | IL-12Rβ2: | Q99665 | TVLPAGDLPTHDGYLPSNIDDLPSHEAPLADSLEELEPQHISLSVF | 195 |
| | STAT4 binding site | | PSSSLHPLTFSCGDKLTLDQLKMRCDSLML | |
| | bold underlined | | | |
| | IL-21R: | Q9HBE5 | SPGDEGPPRSYLRQWVVIPPPLSSPGPQAS* | 206 |
| | STAT3 binding site | | | |
| | bold underlined | | | |
| 7/2/12/ | IL-7Rα: | P16871 | KKRIKPIVWPSLPDHKKTLEHLCKKPRKNLNVSFNPESFLDCQIHR | 193 |
| 21R-2 | Box 1 underlined | | VDDIQARDEVEG | |
| | Box 2 bold | | | |
| | underlined | | | |
| | IL-2Rβ: | P14784 | LERDKVTQLLLQQDKVPEPASLSSNHSLTSCFTNQGYFFFHLPDA | 194 |
| | Shc & STAT5 binding | | LEIEACQVYFTYDPYSEEDPDEGVAGAPTGSSPQPLQPLSGEDDA | |
| | sites bold underlined | | YCTFPSRDDLLLFSPSLLGGPSPPSTAPGGSGAGEERMPPSLQER | |
| | | | VPRDWDPQPLGPPTPGVPDLVDFQPPPELVLREAGEEVPDAGP | |
| | | | REGVSFPWSRPPGQGEFRALNARLPLNTDAYLSLQELQGQDPT | |
| | | | HLV | |
| | IL-12Rβ2: | Q99665 | TVLPAGDLPTHDGYLPSNIDDLPSHEAPLADSLEELEPQHISLSVF | 195 |
| | STAT4 binding site | | PSSSLHPLTFSCGDKLTLDQLKMRCDSLML | |
| | bold underlined | | | |
| | IL-21R: | Q9HBE5 | SPGDEGPPRSYLRQWVVIPPPLSSPGPQAS* | 206 |
| | STAT3 binding site | | | |
| | bold underlined | | | |
| 7/2/21/ | IL-2Rβ: | P14784 | NCRNTGPWLKKVLKCNTPDPSKFFSQLSSEHGGDVQKWLSSPFP | 192 |
| 12R-1 | Box 1 underlined | | SSSFSPGGLAPEISPLEVLERDKVTQLLLQQDKVPEPASLSSNHSLT | |
| | Box 2, Shc, & STAT5 | | SCFTNQGYFFFHLPDALEIEACQVYFTYDPYSEEDPDEGVAGAPT | |
| | binding sites bold | | GSSPQPLQPLSGEDDAYCTFPSRDDLLLFSPSLLGGPSPPSTAPGG | |
| | underlined | | SGAGEERMPPSLQERVPRDWDPQPLGPPTPGVPDLVDFQPPPE | |
| | | | LVLREAGEEVPDAGPREGVSFPWSRPPGQGEFRALNARLPLNTD | |
| | | | AYLSLQELQGQDPTHLV | |
| | IL-21R: | Q9HBE5 | SPGDEGPPRSYLRQWVVIPPPLSSPGPQA | 207 |
| | STAT3 binding site | | | |
| | bold underlined | | | |
| | IL-12Rβ2: | Q99665 | AGDLPTHDGYLPSNIDDLPSHEAPLADSLEELEPQHISLSVFPSSSL | 208 |
| | STAT4 binding site | | HPLTFSCGDKLTLDQLKMRCDSLML* | |
| | bold underlined | | | |
| 7/2/21/ | IL-7Rα: | P16871 | KKRIKPIVWPSLPDHKKTLEHLCKKPRKNLNVSFNPESFLDCQIHR | 193 |
| 12R-2 | Box 1 underlined | | VDDIQARDEVEG | |
| | Box 2 bold | | | |
| | underlined | | | |
| | IL-2Rβ: | P14784 | LERDKVTQLLLQQDKVPEPASLSSNHSLTSCFTNQGYFFFHLPDA | 194 |
| | Shc & STAT5 binding | | LEIEACQVYFTYDPYSEEDPDEGVAGAPTGSSPQPLQPLSGEDDA | |
| | sites bold underlined | | YCTFPSRDDLLLFSPSLLGGPSPPSTAPGGSGAGEERMPPSLQER | |
| | *stop codon | | VPRDWDPQPLGPPTPGVPDLVDFQPPPELVLREAGEEVPDAGP | |
| | | | REGVSFPWSRPPGQGEFRALNARLPLNTDAYLSLQELQGQDPT | |
| | | | HLV | |
| | IL-21R: | Q9HBE5 | SPGDEGPPRSYLRQWVVIPPPLSSPGPQA | 207 |
| | STAT3 binding site | | | |
| | bold underlined | | | |
| | IL-12Rβ2: | Q99665 | AGDLPTHDGYLPSNIDDLPSHEAPLADSLEELEPQHISLSVFPSSSL | 208 |
| | STAT4 binding site | | HPLTFSCGDKLTLDQLKMRCDSLML* | |
| | bold underlined | | | |
| 7/4R-1 | IL-4Rα: Box 1 | P24394 | KIKKEWWDQIPNPARSRLVAIIIQDAQGSQWEKRSRGQEPAKCP | 209 |
| | underlined | | HWKNCLTKLLPCFLEHNMKRDEDPHKAAKEMPFQGSGKSAWC | |
| | Box 2, IRS-2 & STAT6 | | PVEISKTVLWPESISVVRCVELFEAPVECEEEEEVEEEKGSFCASPE | |
| | binding sites bold | | SSRDDFQEGREGIVARLTESLFLDLLGEENGGFCQQDMGESCLLP | |
| | underlined | | PSGSTSAHMPWDEFPSAGPKEAPPWGKEQPLHLEPSPPASPTQ | |
| | | | SPDNLTCTETPLVIAGNPAYRSFSNSLSQSPCPRELGPDPLLARHL | |
| | | | EEVEPEMPCVPQLSEPTTVPQPEPETWEQILRRNVLQHGAAAAP | |
| | | | VSAPTSGYQEFVHAVEQGGTQASAVVGLGPPGEAGYKAFSSLLA | |
| | | | SSAVSPEKCGFGASSGEEGYKPFQDLIPGCPGDPAPVPVPLFTFG | |
| | | | LDREPPRSPQSSHLPSSSPEHLGLEPGEKVEDMPKPPLPQEQATD | |
| | | | PLVDSLGSGIVYSALTCHLCGHLKQCHGQEDGGQTPVMASPCC | |
| | | | GCCCGDRSSPPTTPLRAPDPSPGGVPLEASLCPASLAPSGISEKSK | |
| | | | SSSSFHPAPGNAQSSSQTPKIVNFVSVGPTYMRVS* | |
| 7/4R-2 | IL-7Rα: | P16871 | KKRIKPIVWPSLPDHKKTLEHLCKKPRKNLNVSFNPESFLDCQIHR | 210 |
| | Box 1 underlined | | VDDIQA | |
| | Box 2 bold | | | |
| | underlined | | | |
| | IL-4Rα: | P24394 | DEDPHKAAKEMPFQGSGKSAWCPVEISKTVLWPESISVVRCVEL | 211 |
| | IRS-2 & STAT6 | | FEAPVECEEEEEVEEEKGSFCASPESSRDDFQEGREGIVARLTESL | |
| | binding sites bold | | FLDLLGEENGGFCQQDMGESCLLPPSGSTSAHMPWDEFPSAGP | |
| | underlined | | KEAPPWGKEQPLHLEPSPPASPTQSPDNLTCTETPLVIAGNPAYR | |
| | | | SFSNSLSQSPCPRELGPDPLLARHLEEVEPEMPCVPQLSEPTTVP | |
| | | | QPEPETWEQILRRNVLQHGAAAAPVSAPTSGYQEFVHAVEQGG | |
| | | | TQASAVVGLGPPGEAGYKAFSSLLASSAVSPEKCGFGASSGEEGY | |
| | | | KPFQDLIPGCPGDPAPVPVPLFTFGLDREPPRSPQSSHLPSSSPEH | |
| | | | LGLEPGEKVEDMPKPPLPQEQATDPLVDSLGSGIVYSALTCHLCG | |
| | | | HLKQCHGQEDGGQTPVMASPCCGCCCGDRSSPPTTPLRAPDPS | |
| | | | PGGVPLEASLCPASLAPSGISEKSKSSSSFHPAPGNAQSSSQTPKI | |
| | | | VNFVSVGPTYMRVS* | |
| 7/6R-1 | Gp130: | P40189 | KNKRDLIKKHIWPNVPDPSKSHIAQWSPHTPPRHNFNSKDQMY | 212 |
| | Box 1 underlined | | SDGNFTDVSVVEIEANDKKPFPEDLKSLDLFKKEKINTEGHSSGIG | |
| | Box 2, SHP-2 & | | GSSCMSSSRPSISSSDENESSQNTSSTVQYSTVVHSGYRHQVPSV | |
| | STAT3 binding sites | | QVFSRSESTQPLLDSEERPEDLQLVDHVDGGDGILPRQQYFKQN | |
| | bold underlined | | CSQHESSPDISHFERSKQVSSVNEEDFVRLKQQISDHISQSCGSG | |
| | | | QMKMFQEVSAADAFGPGTEGQVERFETVGMEAATDEGMPKS | |
| | | | YLPQTVRQGGYMPQ* | |
| 7/6R-2 | IL-7Rα: | P16871 | KKRIKPIVWPSLPDHKKTLEHLCKKPRKNLNVSFNPESFLDCQIHR | 213 |
| | Box 1 underlined | | VDDIQARDEVE | |
| | Box 2 bold | | | |
| | underlined | | | |
| | Gp130: | P40189 | KKPFPEDLKSLDLFKKEKINTEGHSSGIGGSSCMSSSRPSISSSDEN | 214 |
| | SHP-2 & STAT3 | | ESSQNTSSTVQYSTVVHSGYRHQVPSVQVFSRSESTQPLLDSEER | |
| | binding sites bold | | PEDLQLVDHVDGGDGILPRQQYFKQNCSQHESSPDISHFERSKQ | |
| | underlined | | VSSVNEEDFVRLKQQISDHISQSCGSGQMKMFQEVSAADAFGP | |
| | | | GTEGQVERFETVGMEAATDEGMPKSYLPQTVRQGGYMPQ* | |

TABLE 34
DOMAIN COMPOSITION OF CHIMERIC CYTOKINE RECEPTORS

| | Signal | | | | | STAT | Other |
|---|---|---|---|---|---|---|---|
| Name | Peptide | ECD | TMD | Box1/2 | ICD | site(s) | site(s) |
| 7/2R-1 | IL-7Rα (190) | IL-7Rα (187) | IL-7Rα (188) | IL-2Rβ (192) | IL-2Rβ (192) | 5 | Shc |
| 7/2R-2 | IL-7Rα (190) | IL-7Rα (187) | IL-7Rα (188) | IL-7Rα (193) | IL-2Rβ (194) | 5 | Shc |
| 7/2/12R-1 | IL-7Rα (190) | IL-7Rα (187) | IL-7Rα (188) | IL-2Rβ (192) | IL-2Rβ (195) | 4, 5 | Shc |
| | | | | | IL-12Rβ2 (195) | | |
| 7/2/12R-2 | IL-7Rα (190) | IL-7Rα (187) | IL-7Rα (188) | IL-7Rα (193) | IL-2Rβ (194) | 4, 5 | Shc |
| | | | | | IL-12Rβ2 (195) | | |
| 7/12/2R-1 | IL-7Rα (190) | IL-7Rα (187) | IL-7Rα (188) | IL-2Rβ (196) | IL-2Rβ (196) | 4, 5 | Shc |
| | | | | | IL-12Rβ2 (197) | | |
| | | | | | IL-2Rβ (198) | | |
| 7/12/2R-2 | IL-7Rα (190) | IL-7Rα (187) | IL-7Rα (188) | IL-7Rα (199) | IL-12Rβ2 (200) | 4, 5 | Shc |
| | | | | | IL-2Rβ (194) | | |
| 7/21-R1 | IL-7Rα (190) | IL-7Rα (187) | IL-7Rα (188) | IL-21R (201) | IL-21R (201) | 3 | N/A |
| 7/21-R2 | IL-7Rα (190) | IL-7Rα (187) | IL-7Rα (188) | IL-7Rα (202) | IL-21R (203) | 3 | N/A |
| 7/21/7R-1 | IL-7Rα (190) | IL-7Rα (187) | IL-7Rα (188) | IL-21R (201) | IL-21R (201) | 3, 5 | PI3K |
| | | | | | IL-7Rα (204) | | |
| 7/7/21R-1 | IL-7Rα (190) | IL-7Rα (187) | IL-7Rα (188) | IL-7Rα (205) | IL-7Rα (205) | 3, 5 | PI3K |
| | | | | | IL-21R (206) | | |
| 7/2/21R-1 | IL-7Ra (190) | IL-7Rα (187) | IL-7Rα (188) | IL-2Rβ (192) | IL-2Rβ (192) | 3, 5 | Shc |
| | | | | | IL-21R (206) | | |
| 7/2/21R-2 | IL-7Rα (190) | IL-7Rα (187) | IL-7Rα (188) | IL-7Rα (193) | IL-2Rβ (194) | 3, 5 | Shc |
| | | | | | IL-21R (206) | | |
| 7/2/12/ | IL-7Rα (190) | IL-7Rα (187) | IL-7Rα (188) | IL-2Rβ (192) | IL-2Rβ (192) | 3, 4, 5 | Shc |
| 21R-1 | | | | | IL-12Rβ2 (195) | | |
| | | | | | IL-21R (206) | | |
| 7/2/12/ | IL-7Rα (190) | IL-7Rα (187) | IL-7Rα (188) | IL-7Rα (193) | IL-2Rβ (194) | 3, 4, 5 | Shc |
| 21R-2 | | | | | IL-12Rβ2 (195) | | |
| | | | | | IL-21R (206) | | |
| 7/2/21/ | IL-7Rα (190) | IL-7Rα (187) | IL-7Rα (188) | IL-2Rβ (192) | IL-2Rβ (192) | 3, 4, 5 | Shc |
| 12R-1 | | | | | IL-21R (207) | | |
| | | | | | IL-12Rβ2 (208) | | |
| 7/2/21/ | IL-7Rα (190) | IL-7Rα (187) | IL-7Rα (188) | IL-7Rα (193) | IL-2Rβ (194) | 3, 4, 5 | Shc |
| 12R-2 | | | | | IL-21R (207) | | |
| | | | | | IL-12Rβ2 (208) | | |
| 7/4R-1 | IL-7Rα (190) | IL-7Rα (187) | IL-7Rα (188) | IL-4Rα (209) | IL-4Rα (209) | 6 | IRS-2 |
| 7/4R-2 | IL-7Rα (190) | IL-7Rα (187) | IL-7Rα (188) | IL-7Rα (210) | IL-7Rα (210) | 6 | IRS-2 |
| | | | | | IL-4Rα (211) | | |
| 7/6R-1 | IL-7Rα (190) | IL-7Rα (187) | IL-7Rα (188) | gp130 (212) | gp130 (212) | 3 | SHP-2 |
| 7/6R-2 | IL-7Rα (190) | IL-7Rα (187) | IL-7Rα (188) | IL-7Rα (213) | IL-7Rα (213) | 3 | SHP-2 |
| | | | | | gp130 (214) | | |

TABLE 35
CONTROLLED PARACRINE SIGNALING AND DETECTION OF SECRETED CYTOKINES IN MURINE OT-I
T CELLS TRANSDUCED WITH RETROVIRAL VECTORS ENCODING CHIMERIC CYTOKINE RECEPTORS

| Receptor | Cytokine | IL-18 | Eotaxin | G-CSF | GM-CSF | IFN-g | IL-1a | IL-1b | IL-2 | IL-3 | IL-4 | IL-5 |
|---|---|---|---|---|---|---|---|---|---|---|---|---|
| G2R-2 | Medium | OOR< | OOR< | 0.79 | 3.31 | 0.95 | 0.58 | 0.43 | OOR< | 1.25 | 0.12 | OOR< |
| G2R-2 | IL-2 | OOR< | OOR< | 1.08 | 13.70 | 1.32 | OOR< | 0.14 | OOR< | 0.35 | 0.14 | 0.57 |
| G2R-2 | PEG-130a1 | OOR< | 0 96 | 0.91 | 17.70 | 2.29 | 2.51 | OOR< | OOR< | 0.35 | 0.12 | 1.09 |
| G2R-3 | Medium | OOR< | OOR< | 0.86 | 3.34 | 0.83 | OOR< | 029 | OOR< | 0.86 | 0.17 | OOR< |
| G2R-3 | IL-2 | OOR< | OOR< | 1.01 | 14.09 | 0.86 | OOR< | 0.14 | OOR< | 0.45 | 0.15 | 0.30 |
| G2R-3 | PEG-130a1 | OOR< | OOR< | 1.01 | 14.52 | 0.95 | OOR< | OOR< | OOR< | 0.35 | 0.12 | 0.06 |
| G12/2R-1 | Medium | OOR< | OOR< | 0.72 | 3.34 | 1.20 | OOR< | OOR< | OOR< | 0.92 | 0.16 | OOR< |
| G12/2R-1 | IL-2 + IL-12 | OOR< | OOR< | 0.79 | 95.76 | 717.93 | 7.94 | 029 | OOR< | OOR< | 0.18 | OOR< |
| G12/2R-1 | PEG-130a1 | OOR< | OOR< | 1.38 | 15.79 | 2.00 | 0.96 | 0.43 | OOR< | 0.19 | 0.16 | OOR< |
| G12/2R-1 + | Medium | 155.62 | 0.18 | 0.86 | 12.20 | 6.92 | OOR< | OOR< | OOR< | 0.17 | 021 | OOR< |
| m18 | | | | | | | | | | | | |
| G12/2R-1 + | IL-2 + IL-12 | 288.24 | OOR< | 1.52 | 972.75 | 9871.71 | 40.19 | OOR< | 0.59 | 0.86 | 023 | 0.81 |
| m18 | | | | | | | | | | | | |
| G12/2R-1 + | PEG-130a1 | 811.10 | OOR< | 1.27 | 271.81 | 7426.63 | 19.56 | OOR< | OOR< | 0.47 | 020 | 7.59 |
| m18 | | | | | | | | | | | | |
| Receptor | Cytokine | IL-6 | IL-7 | IL-9 | IL-10 | IL-12p40 | IL-12p70 | IL-13 | IL-15 | IL-17 | IP-10 | KC |
| G2R-2 | Medium | 0.24 | 025 | OOR< | 2.61 | OOR< | 3.19 | OOR< | 1.28 | OOR< | 2.46 | 0.96 |
| G2R-2 | IL-2 | 0.38 | 0.54 | OOR< | 52.28 | OOR< | 1.7 | OOR< | OOR< | 0.10 | 2.97 | 2.48 |
| G2R-2 | PEG-130a1 | 0.27 | 0.74 | OOR< | 300.01 | OOR< | 526 | OOR< | 2.56 | 0.28 | 3.59 | 1.20 |
| G2R-3 | Medium | 0.34 | 0.30 | OOR< | 3.29 | OOR< | 1.73 | OOR< | OOR< | 0.07 | 1.98 | 0.57 |
| G2R-3 | IL-2 | 0.33 | 0.19 | OOR< | 44.22 | OOR< | 2.59 | OOR< | OOR< | OOR< | 2.74 | 0.55 |
| G2R-3 | PEG-130a1 | 0.28 | 0.08 | OOR< | 68.26 | OOR< | 2.17 | OOR< | OOR< | 0.03 | 2.90 | 0.21 |
| G12/2R | Medium | 0.49 | 023 | OOR< | 2.43 | OOR< | 1.90 | OOR< | OOR< | OOR< | 2.76 | 0.55 |
| G12/2R | IL-2 + IL-12 | 0.33 | 0.11 | 020 | 1402.50 | 24.79 | 1166.81 | OOR< | OOR< | 0.10 | 4.29 | 0.96 |
| G12/2R | PEG-130a1 | 0.29 | 0.05 | 020 | 160.37 | OOR< | 2.79 | OOR< | OOR< | OOR< | 3.26 | 8.23 |
| G12/2R/18C | Medium | 0.48 | 0.30 | OOR< | 12.99 | OOR< | 1.77 | OOR< | 0.65 | 0.47 | 8.95 | 1.05 |
| G12/2R/18C | IL-2 + IL-12 | 18.08 | 0.54 | 0.39 | 2911.40 | 19.69 | 875.39 | OOR< | OOR< | 1.51 | 18.77 | 2.57 |
| G12/2R/18C | PEG-130a1 | 1.94 | 0.53 | OOR< | 1202.95 | OOR< | 2.99 | OOR< | 0.97 | 0.79 | 11.07 | 0.44 |
| Receptor | Cytokine | LIF | LIX | MCP-1 | M-CSF | MG | MP-1a | MP-1b | MP-2 | RANTES | TNFa | VEGF |
| G2R-2 | Medium | 11.58 | 96.40 | 5.13 | 0.28 | 0.02 | 4.19 | OOR< | OOR< | 0.33 | 2.69 | 3.67 |
| G2R-2 | IL-2 | 11.91 | 80.86 | 320 | 0.73 | 0.02 | OOR< | OOR< | 20.29 | 1.64 | 6.56 | 45.30 |
| G2R-2 | PEG-130a1 | 15.25 | 87.57 | 0.99 | 0.23 | 0.04 | 4.02 | 12.86 | 33.70 | 3.12 | 10.33 | 78.42 |
| G2R-3 | Medium | 7.70 | 53.21 | 6.51 | 0.28 | 0.04 | OOR< | OOR< | 15.72 | 0.48 | 3.01 | 6.90 |
| G2R-3 | IL-2 | 7.55 | 78.33 | 4.30 | 0.46 | OOR< | 3.38 | OOR< | 13.24 | 1.60 | 5.45 | 40.10 |
| G2R-3 | PEG-130a1 | 8.86 | 104.04 | 4.86 | 0.32 | OOR< | OOR< | OOR< | OOR< | 1.95 | 7.34 | 76.88 |
| G12/2R | Medium | 5.38 | 69.45 | 5.96 | 0.46 | OOR< | OOR< | 5.61 | 37.16 | 1.14 | 2.44 | 2.88 |
| G12/2R | IL-2 + IL-12 | 12.60 | 80.50 | 9.57 | 0.82 | 0.18 | 7.98 | 3.52 | 7.05 | 1.92 | 7.12 | 38.94 |
| G12/2R | PEG-130a1 | 7.07 | 117.52 | 4.86 | 0.73 | 0.07 | 3.38 | OOR< | 33.70 | 2.82 | 6.57 | 56.95 |
| G12/2R/18C | Medium | 13.79 | 70.40 | 4.58 | 0.82 | 0.06 | 3.38 | OOR< | 39.25 | 5.58 | 6.77 | 8.30 |
| G12/2R/18C | IL-2 + IL-12 | 39.84 | 88.99 | 8.18 | 0.60 | 0.39 | 7.98 | 7.97 | 26.47 | 6.46 | 26.12 | 121.10 |
| G12/2R/18C | PEG-130a1 | 17.54 | 85.52 | 7.07 | 0.87 | 0.08 | OOR< | OOR< | 20.86 | 8.46 | 16.67 | 78.03 |

1-303. (canceled)

304. A variant Granulocyte Colony-Stimulating Factor (G-CSF), wherein
the variant G-CSF comprises at least one mutation in a site II interface region, at least one mutation in a site III interface region, or a combination thereof;
wherein the at least one mutation in the site II interface region comprises at least one of: L108K, D112R, E122R E122K, E123K, and E123R relative to the corresponding amino acid positions of the sequence shown in SEQ ID NO. 1;
wherein the at least one mutation in the site III interface region comprises mutation E46R relative to the corresponding amino acid positions of the sequence shown in SEQ ID NO. 1; and wherein the variant G-CSF binds selectively to a receptor comprising a variant extracellular domain (ECD) of Granulocyte Colony-Stimulating Factor Receptor (G-CSFR).

305. The variant G-CSF of claim 304, wherein the receptor comprising the variant ECD of G-CSFR comprises at least one mutation in a site II interface region, at least one mutation in a site III interface region, or a combination thereof; optionally wherein the at least one mutation in the site II interface region of the G-CSFR ECD comprises one or both of a R141E or a R167D mutation; and wherein the at least one mutation in the site III interface region of the G-CSFR ECD comprises a R41E mutation; and wherein the variant G-CSFR mutations correspond to an amino acid position of the sequence shown in SEQ ID NO. 2; and optionally wherein the receptor comprising the variant ECD of G-CSFR is a chimeric receptor; or the G-CSF is chemically modified.

306. A system for selective activation of a receptor expressed on a cell surface, the system comprising:
(a) the variant G-CSF of claim 304; and
(b) a receptor comprising a variant ECD of G-CSFR; wherein
the variant G-CSF binds selectively to a receptor comprising a variant ECD of G-CSFR; and, optionally,
(c) one or more additional agonistic or antagonistic signaling proteins or antigen binding signaling receptors; wherein
i) the variant G-CSF comprises mutations E46R, L108K, D112R, E122R, and E123R relative to the corresponding amino acid positions of the sequence shown in SEQ ID NO. 1; and wherein the receptor comprising a variant ECD of G-CSFR comprises mutations R41E, R141E and R167D relative to the corresponding amino acid positions of the sequence shown in SEQ ID NO. 2;
ii) the variant G-CSF comprises mutations E46R, L108K, D112R, and E122K relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2;
iii) the variant G-CSF comprises mutations E46R, L108K, D112R, and E123K relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2;
iv) the variant G-CSF comprises mutations E46R, L108K, D112R, and E122R relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2;
v) the variant G-CSF comprises mutations E46R, L108K, D112R, and E123R relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2;
vi) the variant G-CSF comprises mutations E46R, L108K, D112R, E122K, and E123K relative to the corresponding amino acid positions of the sequence shown in SEQ ID NO. 1; and wherein the receptor comprising a variant ECD of G-CSFR comprises mutations R41E, R141E, and R167D relative to the corresponding amino acid positions of the sequence shown in SEQ ID NO. 2; or
vii) the variant G-CSF corresponds to SEQ ID NO: 83 or 84.

307. A method of increasing an immune response in a subject in need thereof or treating a disease in the subject in need thereof, comprising administering cells expressing a receptor comprising a variant ECD of G-CSFR and administering or providing the variant G-CSF of claim 304, wherein optionally:
i) the method involves a subject being administered one or more populations of cells each expressing one or both of: (i) a distinct chimeric receptor comprising a G-CSFR ECD or (ii) at least one distinct variant form of G-CSF; optionally wherein at least one population of cells further express at least one distinct antigen binding signaling receptor or additional agonistic or antagonistic signaling protein;
ii) the method involves administering to a subject one or more additional agonistic of antagonistic signaling protein;
iii) the method comprises: (i) isolating an immune cell-containing sample; (ii) transducing or transfecting the immune cells with a nucleic acid encoding a receptor comprising a variant ECD of G-CSFR; (iii) administering the immune cells from (ii) to the subject; and (iv) contacting the immune cells with a variant G-CSF of claim 304 that selectively binds the receptor; or
iv) the method comprises the cells are contacted with the variant G-CSF in vitro prior to administering the cells to the subject.

308. A kit for producing the system of claim 306, comprising:
a. cells encoding a receptor comprising a variant ECD of G-CSFR; and instructions for use; and a variant G-CSF of claim 304; or one or more nucleic acids encoding a receptor comprising the variant ECD of G-CSFR, and the variant G-CSF of claim 304; and
b. instructions for use;
and optionally wherein
the kit further comprises one or more expression vectors or cells that encode one or more cytokines or chemokines or antigen binding receptors.

309. A nucleic acid encoding the variant G-CSF of claim 304 and the receptor comprising the variant ECD of G-CSFR.

310. A vector comprising the nucleic acid of claim 309.

311. A chimeric receptor, comprising:
(a) an ECD operatively linked to at least one second domain, the second domain comprising:
(b) an ICD comprising at least one signaling molecule binding site from an intracellular domain of a cytokine receptor; wherein
the at least one signaling molecule binding site is selected from the group consisting of: a SHC binding site of Interleukin (IL)-2Rβ; a STAT5 binding site of IL-2Rβ, an IRS-1 or IRS-2 binding site of IL-4Rα, a STAT6 binding site of IL-4Rα, a SHP-2 binding site of glycoprotein 130 (gp130), a STAT3 binding site of gp130, a SHP-1 or SHP-2 binding site of Erythropoietin Receptor (EPOR), a STAT5 binding site of EPOR, a STAT1 or STAT2 binding site of Interferon Alpha And Beta Receptor Subunit 2 (IFNAR2), and a STAT1 binding site of Interferon Gamma Receptor 1 (IFNγR1), or combinations thereof;
wherein
the ICD further comprises at least one Box 1 region and at least one Box 2 region of at least one protein selected from the group consisting of G-CSFR, gp130, EPOR, and Interferon Gamma Receptor 2 (IFNγR2), or combinations thereof; and
(c) at least one third domain comprising a TMD; wherein the ECD is N-terminal to the TMD, and the TMD is N-terminal to the ICD.

312. The chimeric receptor of claim 311, wherein an activated form of the chimeric receptor forms a homodimer, and, optionally, activation of the chimeric receptor causes a cellular response comprising at least one of proliferation, viability, persistence, cytotoxicity, cytokine secretion, memory, and enhanced activity of a cell expressing the receptor, and, optionally, the chimeric receptor is activated upon contact with a G-CSF.

313. The chimeric receptor of claim 312, wherein the G-CSF is a wild-type G-CSF, and, the extracellular domain of the G-CSFR is a wild-type extracellular domain

314. The chimeric receptor of claim 311, wherein
the ECD is an ECD of G-CSFR, optionally wherein the ECD of G-CSFR binds a variant G-CSF, wherein:
(i) the variant G-CSF comprises at least one mutation in a site II interface region, at least one mutation in a site III interface region, or combinations thereof; wherein the at least one mutation in the site II interface region comprises at least one of: L108K, D112R, E122R, E122K, E123K, and E123R, and combinations thereof relative to the corresponding amino acid positions of the sequence shown in SEQ ID NO. 1; wherein the at least one mutation in the site III interface region comprises mutation E46R relative to the corresponding amino acid positions of the sequence shown in SEQ ID NO. 1; and wherein the variant G-CSF binds selectively to a receptor comprising a variant extracellular domain (ECD) of Granulocyte Colony-Stimulating Factor Receptor (G-CSFR).
(ii) the variant ECD of G-CSFR comprises at least one mutation in a site II interface region, at least one mutation in a site III interface region, or combinations thereof; optionally wherein
the at least one mutation in the site II interface region of the G-CSFR ECD comprises one or both of a R141E or a R167D mutation; and wherein
the at least one mutation in the site III interface region of the G-CSFR ECD comprises a R41E mutation; and wherein
the variant G-CSFR mutations correspond to an amino acid position of the sequence shown in SEQ ID NO. 2; or wherein
the TMD is a TMD of G-CSFR; or wherein
an activated form of the chimeric receptor forms a homodimer; or wherein
the chimeric receptor is activated upon contact with a G-CSF; or wherein
the chimeric receptor is expressed in a cell, preferably an immune cell; or wherein
the ICD comprises:
(a) an amino acid sequence of one or both of SEQ ID NO. 90 or 91; or
(b) an amino acid sequence of one or both of SEQ ID NO. 90 or 92; or
(c) an amino acid sequence of SEQ ID NO. 93; or
(d) an amino acid sequence of SEQ ID NO. 94; or
(e) an amino acid sequence of one or both of SEQ ID NO. 95 or 96; or
(f) an amino acid sequence of SEQ ID NO. 97 or 98; or
(g) an amino acid sequence of SEQ ID NO. 99 or 100; or wherein
the TMD comprises a sequence set forth in SEQ ID NO. 88; or wherein
the TMD is a TMD of G-CSFR; or wherein
the TMD is a wild-type TMD.

315. A system for selective activation of a receptor expressed on a cell surface, the system comprising:
(a) a variant G-CSF or the variant G-CSF of claim 314, one or more nucleic acids encoding the variant G-CSF, or one or more expression vectors comprising the one or more nucleic acids encoding the variant G-CSF; and
(b) the receptor comprising a variant ECD of G-CSFR of claim 314, one or more nucleic acids encoding the receptor, or one or more expression vectors comprising the one or more nucleic acids encoding the receptor.

316. A cell comprising one or more nucleic acids encoding the chimeric receptor of claim 311, or one or more expression vectors comprising the one or more nucleic acids encoding the chimeric receptor of claim 311, wherein the cell is preferably an immune cell.

317. A method of selective activation of a chimeric receptor expressed on the surface of a cell, preferably an immune cell, comprising contacting the chimeric receptor of claim 311 with a cytokine that selectively binds the chimeric receptor; optionally wherein an activated form of the chimeric receptor forms a homodimer; or
activation of the chimeric receptor causes a cellular response; or
the chimeric receptor is activated upon contact with a variant G-CSF; or
a first population of immune cells expresses the chimeric receptor and a second population of immune cells express a cytokine that binds the chimeric receptor, optionally wherein
one or both populations of immune cells further express at least one distinct antigen binding signaling receptor, additional agonistic or antagonistic signaling protein, or antigen binding signaling receptor; or
wherein each of the first and second populations of immune cells express a distinct chimeric receptor comprising a distinct variant ECD of G-CSFR and a distinct variant G-CSF.

318. A method of treating a subject in need thereof, comprising administering to the subject a cell, preferably an immune cell, expressing the chimeric receptor of claim 311, and providing to the subject a cytokine that specifically binds the chimeric receptor; optionally wherein
an activated form of the chimeric receptor forms a homodimer; or
activation of the chimeric receptor causes a cellular response; or
wherein the cytokine is G-CSF; wherein optionally
i) the method comprises administering at least one additional agonistic or antagonistic signaling protein;
ii) the method comprises administering to the subject two or more populations of cells each expressing a distinct chimeric receptor and each expressing a distinct variant form of a cytokine, and optionally expressing at least one antigen binding signaling receptor; or
iii) the method comprises: (i) isolating an immune cell-containing sample; (ii) introducing to the immune cell a nucleic acid encoding a receptor comprising a variant ECD of G-CSFR; (iii) administering the immune cell from (ii) to the subject; and (iv) contacting the immune cell with the cytokine, optionally G-CSF, that binds the receptor.

319. A kit comprising:
(a) cells, preferably immune cells, comprising one or more chimeric receptors of claim 311 or one or more expressions vectors encoding one or more chimeric receptors;
(b) instructions for use; and, optionally
(c) at least one cytokine that binds the chimeric receptor or one or more expression vectors that encode one or more cytokines or chemokines, or antigen binding receptors, or additional agonistic or antagonistic signaling protein, or antigen binding signaling receptor, or distinct chimeric receptor of claim 311.

320. A nucleic acid encoding the chimeric receptor of claim 311.

321. A vector comprising the nucleic acid of claim 320.

322. A method for gene editing in a cell, the method comprising: introducing into the genome of the cell the nucleic acid of claim 320.

323. A method for treating cancer in a subject in need thereof, the method comprising administering a therapeutically effective amount of the chimeric receptor of claim 311.

324. A method for treating an autoimmune disease or disorder in a subject in need thereof, the method comprising administering a therapeutically effective amount of the chimeric receptor of claim 311.

325. A system for selective activation of a cell, optionally an immune cell, the system comprising:
(i) a receptor comprising a variant ECD of G-CSFR;
(ii) a variant G-CSF that selectively binds the receptor of (i); and
(iii) one or both of:
(a) at least one additional agonistic or antagonistic signaling protein; and
(b) at least one antigen binding signaling receptor; wherein the variant ECD of G-CSFR comprises at least one mutation in a site II interface region, at least one mutation in a site III interface region, or combinations thereof; wherein
i) the at least one mutation in the site II interface region is located at an amino acid position of the G-CSFR ECD selected from the group consisting of amino acid position 141, 167, 168, 171, 172, 173, 174, 197, 199, 200, 202 and 288 of the sequence shown in SEQ ID NO. 2;
ii) the at least one mutation in the site II interface region of the G-CSFR ECD is selected from the group consisting of R141E, R167D, K168D, K168E, L171E, L172E, Y173K, Q174E, D197K, D197R, M199D, D200K, D200R, V202D, R288D, and R288E of the sequence shown in SEQ ID NO. 2;
iii) the at least one mutation in the site III interface region of the G-CSFR ECD is selected from the group consisting of amino acid position 30, 41, 73, 75, 79, 86, 87, 88, 89, 91, and 93 of amino acids 2-308 of the sequence shown in SEQ ID NO. 2; or
iv) the at least one mutation in the site III interface region of the G-CSFR ECD is selected from the group consisting of S30D, R41E, Q73W, F75KF, S79D, L86D, Q87D, I88E, L89A, Q91D, Q91K, and E93K of the sequence shown in SEQ ID NO. 2; or
wherein the G-CSFR ECD comprises the mutations: R41E, R141E, and R167D of the sequence shown in SEQ ID NO. 2.

326. The system of claim 325, wherein
the variant G-CSF comprises at least one mutation in a site II interface region, at least one mutation in a site III interface region, or combinations thereof; wherein:
i) the at least one mutation in the site II interface region of the variant G-CSF is located at an amino acid position selected from the group consisting of amino acid position 12, 16, 19, 20, 104, 108, 109, 112, 115, 116, 118, 119, 122 and 123 of the sequence shown in SEQ ID NO. 1;
ii) the at least one mutation in the site II interface region of the variant G-CSF is selected from a group of mutations selected from the group consisting of: S12E, S12K, S12R, K16D, L18F, E19K, Q20E, D104K, D104R, L108K, L108R, D109R, D112R, D112K, T115E, T115K, T116D, Q119E, Q119R, E122K, E122R, and E123R;
iii) the at least one mutation in the site III interface region of the variant G-CSF is selected from a group of mutations selected from the group consisting of: 38, 39, 40, 41, 46, 47, 48, 49, and 147 of the sequence shown in SEQ ID NO. 1; or
iv) the at least one mutation in the site III interface region of the variant G-CSF is selected from a group of mutations selected from the group consisting of: T38R, Y39E, K40D, K40F, L41D, L41E, L41K, E46R, L47D, V48K, V48R, L49K, and R147E.

327. A method of selective activation of a receptor comprising a variant ECD of G-CSFR expressed on the surface of a cell, preferably an immune cell or for producing a cell expressing the receptor, comprising:
a) introducing into the cell one or more one or more nucleic acids encoding at least one receptors comprising a variant ECD of G-CSFR of the system of claim 325; and one or both of
(i) at least one additional agonistic or antagonistic signaling protein; and
(ii) at least one antigen binding signaling receptor of the system; and
b) contacting the receptor comprising the variant ECD of G-CSFR with a variant G-CSF or the variant G-CSF; and optionally wherein
a first population of immune cells expresses one or more receptors comprising the variant ECD of G-CSFR and a second population of immune cells express one or more variant G-CSF; optionally, wherein one or both populations of immune cells further express at least one distinct antigen binding signaling receptor, additional agonistic or antagonistic signaling protein; or antigen binding signaling receptor.

328. A method for increasing an immune response or treating a disease in a subject in need thereof, comprising
a) isolating an immune cell-containing sample;
b) introducing a nucleic acid encoding the variant ECD of G-CSFR of the system of claim 325, and optionally at least one additional active agent antigen binding signaling receptor;
c) administering the immune cells from b) to the subject; and
d) contacting the immune cells with a variant G-CSF; wherein the cells are optionally contacted with a variant G-CSF or additional cytokine or chemokine in vitro prior to administering the cells to the subject.

329. A kit, comprising:
a) cells, optionally immune cells, comprising:
the receptor comprising the variant ECD of G-CSFR of the system of claim 325, or comprising one or more nucleic acids or expression vectors encoding the receptor comprising the variant ECD of G-CSFR of the system of claim 325; and one or both of:
i) the at least one additional cytokine and chemokine; and ii) the at least one antigen binding signaling receptor;
b) and instructions for use; and
c) optionally, at least one variant G-CSF that optionally specifically binds the receptor comprising the variant ECD of G-CSFR.

330. A chimeric receptor, comprising:
(i) an ECD of Interleukin Receptor alpha (IL-7Rα);
(ii) a TMD; and
(iii) an ICD of a cytokine receptor that is distinct from a wild-type, human IL-7Rα intracellular signaling domain set forth in SEQ ID NO:109; wherein the ECD and TMD are each operatively linked to the ICD.

331. The chimeric receptor of claim 330, wherein:
the C-terminus of the ECD is linked to the N-terminus of the TMD, and the C-terminus of TMD is linked to the N-terminus of the ICD;
the ECD is the ECD of native human IL-7Rα;
the TMD is the TMD of IL-7Rα;
the TMD is the TMD of native human IL-7Rα; or
the ICD comprises at least one signaling molecule binding site from an intracellular domain of a cytokine receptor; and, optionally,
the at least one signaling molecule binding site comprises:
(a) a Jak 1 binding site (Box 1 and 2 region) of IL-2Rβ, IL-4Ra, IL-7Ra, IL-21R, or gp130;
(b) a SHC binding site of IL-2Rβ;
(c) a STAT5 binding site of IL-2Rβ or IL-7Ra;
(d) a STAT3 binding site of IL-21R or gp130;
(e) a STAT4 binding site of IL-12Rβ2;
(f) a STAT6 binding site of IL-4Ra;
(g) an IRS-1 or IRS-2 binding site of IL-4Ra; and
(h) a SHP-2 binding site of gp130;
(i) a PI3K binding site of IL-7Rα;
or combinations thereof; or optionally
the ICD comprises at least an intracellular signaling domain of a receptor that is activated by heterodimerization with the common gamma chain (gc); or optionally
the ICD comprises at least an intracellular signaling domain of a cytokine receptor selected from the group consisting of: IL-2Rβ (Interleukin-2 receptor beta), IL-4Rα (Interleukin-4 Receptor alpha), IL-9Rα (Interleukin-9 Receptor alpha), IL-12R (Interleukin-12 Receptor), IL-21R (Interleukin-21 Receptor) and gp130, and combinations thereof.

332. The chimeric receptor of claim 330, wherein the chimeric receptor comprises an ECD of IL-7Rα and a TMD operatively linked to an ICD, the ICD comprising:
(i)
(a) a Box 1 and a Box 2 region of IL-2Rβ;
(b) a SHC binding site of IL-2Rβ; and
(c) a STAT5 binding site of IL-2Rβ; or
(ii)
(a) a Box 1 and a Box 2 region of IL-7Rα;
(b) a SHC binding site of IL-2Rβ; and
(c) a STAT5 binding site of IL-2Rβ; or
(iii)
(a) a Box 1 and a Box 2 region of IL-2Rβ;
(b) a SHC binding site of IL-2Rβ;
(c) a STAT5 binding site of IL-2Rβ; and
(d) a STAT4 binding site of IL-12Rβ2; or
(iv)
(a) a Box 1 and a Box 2 region of IL-7Rα;
(b) a SHC binding site of IL-2Rβ;
(c) a STAT5 binding site of IL-2Rβ; and
(d) a STAT4 binding site of IL-12Rβ2; or
(v)
(a) a Box 1 and a Box 2 region of IL-21R; and
(b) a STAT3 binding site of IL-21R; or
(vi)
(a) a Box 1 and a Box 2 region of IL-7Rα; and
(b) a STAT3 binding site of IL-21R; or
(vii)
(a) a Box 1 and a Box 2 region of IL-21R;
(b) a STAT3 binding site of IL-21R; and
(c) a STAT5 and PI3 kinase binding site of IL-7Rα;
(viii)
(a) a Box 1 and a Box 2 region of IL-7Rα;
(b) a STAT3 binding site of IL-21R; and
(c) a STAT5 and PI3 kinase binding site of IL-7Rα;
(ix)
(a) a Box 1 and a Box 2 region of IL-2Rβ;
(b) a SHC binding site of IL-2Rβ;
(c) a STAT5 binding site of IL-2Rβ; and
(d) a STAT3 binding site of IL-21R; or
(x)
(a) a Box 1 and a Box 2 region of IL-7Rα;
(b) a SHC binding site of IL-2Rβ;
(c) a STAT5 binding site of IL-2Rβ; and
(d) a STAT3 binding site of IL-21R; or
(xi)
(a) a Box 1 and a Box 2 region of IL-2Rβ;
(b) a SHC binding site of IL-2Rβ;
(c) a STAT5 binding site of IL-2Rβ;
(d) a STAT4 binding site of IL12Rβ2; and
(e) a STAT3 binding site of IL-21R; or
(xii)
(a) a Box 1 and a Box 2 region of IL-7Rα;
(b) a SHC binding site of IL-2Rβ;
(c) a STAT5 binding site of IL-2Rβ;
(d) a STAT4 binding site of IL12Rβ2; and
(e) a STAT3 binding site of IL-21R; or
(xiii)
(a) a Box 1 and a Box 2 region of IL-4Rα;
(b) an IRS-1 or IRS-2 binding site of IL-4Rα; and
(c) a STAT6 binding site of IL-4Rα; or
(xiv)
(a) a Box 1 and a Box 2 region of IL-7Rα;
(b) an IRS-1 or IRS-2 binding site of IL-4α; and
(c) a STAT6 binding site of IL-4α; or
(xv)
(a) a Box 1 and a Box 2 region of gp130;
(b) a SHP-2 binding site of gp130; and
(c) a STAT3 binding site of gp130; or
(xvi)
(a) a Box 1 and a Box 2 region of IL-7α;
(b) a SHP-2 binding site of gp130; and
(c) a STAT3 binding site of gp130; and optionally wherein
(b) is N-terminal to (c); or wherein
(c) is N-terminal to (b); or wherein
(c) is N-terminal to (d); or wherein
(d) is N-terminal to (c); or wherein
(d) is N-terminal to (e); or wherein
(e) is N-terminal to (d); or optionally
wherein the ICD comprises a sequence at least 80% identical to a sequence shown in at least one of SEQ ID NO: 85-107.

333. A system for activation of a receptor expressed on a cell surface, the system comprising:
(a) the chimeric receptor of claim 330; and
(b) IL-7; and optionally
(c) an antigen binding signaling receptor; and optionally
(d) at least one additional agonistic or antagonistic signaling protein; and, optionally,
(e) at least one antigen binding signaling receptor.

334. A method of activation of a chimeric receptor expressed on the surface of a cell, preferably an immune cell, comprising:
contacting the chimeric receptor of claim 330; or one or more nucleic acids or one or more vectors encoding the chimeric receptor of claim 330.

335. A kit, comprising:
a. cells comprising a chimeric receptor of claim 330, or one or more nucleic acids sequences or one or more expression vectors encoding at least one cytokine or chemokine, or at least one antigen binding signaling receptor, or at least one CAR; and
b. instructions for use; and, optionally,
c. IL-7; and optionally
(d) a variant G-CSF.

336. A nucleic acid encoding the chimeric receptor of claim 330.

337. A vector comprising the nucleic acid of claim 336.


SEQUENCE LISTING (237 sequences)

SEQ ID NO: 1 - protein, 174 aa, Artificial Sequence
  G-CSF
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 308 aa, Artificial Sequence
  G-CSFR (Ig-CRH)
EECGHISVSAPIVHLGDPITASCIIKQNCSHLDPEPQILWRLGAELQPGGRQQRLSDGTQESIITLPHLNHTQAFLSCSLNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTE

SEQ ID NO: 3 - protein, 627 aa, Artificial Sequence
  G-CSFR (Signal peptide, ECD)
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELH

SEQ ID NO: 4 - protein, 123 aa, Artificial Sequence
  gp130 (TM, ICD)
AIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSIS

SEQ ID NO: 5 - protein, 205 aa, Artificial Sequence
  IL-2R beta ICD)
ASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 627 aa, Artificial Sequence; identical to SEQ ID NO: 3
  G-CSFR (Signal peptide, ECD)

SEQ ID NO: 7 - protein, 328 aa, Artificial Sequence
  IL-2R beta (TM and ICD)
AIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 8 - protein, 627 aa, Artificial Sequence; identical to SEQ ID NO: 3
  G-CSFR (Signal peptide, ECD)

SEQ ID NO: 9 - protein, 107 aa, Artificial Sequence
  IL-2R gamma (TM and ICD)
VVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 10 - skipped

SEQ ID NO: 11 - protein, 24 aa, Artificial Sequence
  G-CSFR (Signal peptide)
MARLGNCSLTWAALIILLLPGSLE

SEQ ID NO: 12 - protein, 22 aa, Artificial Sequence
  GM-CSFR-alpha (Signal peptide)
MLLLVTSLLLCELPHPAFLLIP

SEQ ID NO: 13 - DNA, 72 bp, Artificial Sequence
  G-CSFR (Signal peptide, native sequence)
atggcaaggctgggaaactgcagcctgacttgggctgccctgatcatcctgctgctccccggaagtctggag

SEQ ID NO: 14 - DNA, 66 bp, Artificial Sequence
  GM-CSFR-alpha (Signal peptide, native sequence)
atgctgctgctagtgacctccctgctgctctgtgagctgcctcacccggcgttcctgctgattcct

SEQ ID NO: 15 - protein, 603 aa, Artificial Sequence
  G-CSFR (ECD)
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELH

SEQ ID NO: 16 - DNA, 1809 bp, Artificial Sequence
  G-CSFR (ECD, native sequence)
gagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacac

SEQ ID NO: 17 - DNA, 1809 bp, Artificial Sequence
  G-CSFR (ECD, 4 codons altered)
gagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacgcatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgccccaccccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcatcctcccgtggctttgtcctccatggcctggagcccgccagtctgtatcacatccacctcatggctgccagccaggctggggccaccaacagtacagtcctcaccctgatgaccttgaccccagaggggtcggagctacac

SEQ ID NO: 18 - protein, 23 aa, Artificial Sequence
  G-CSFR (TM)
IILGLFGLLLLLTCLCGTAWLCC

SEQ ID NO: 19 - protein, 22 aa, Artificial Sequence
  gp130 (TM)
AIVVPVCLAFLLTTLLGVLFCF

SEQ ID NO: 20 - protein, 25 aa, Artificial Sequence
  IL-2Rb (TM)
IPWLGHLLVGLSGAFGFIILVYLLI

SEQ ID NO: 21 - protein, 21 aa, Artificial Sequence
  IL-2RG (TM)
VVISVGSMGLIISLLCVYFWL

SEQ ID NO: 22 - DNA, 69 bp, Artificial Sequence
  G-CSFR (TM)
atcatcctgggcctgttcggcctcctgctgttgctcacctgcctctgtggaactgcctggctctgttgc

SEQ ID NO: 23 - DNA, 66 bp, Artificial Sequence
  gp130 (TM)
gccatagtcgtgcctgtttgcttagcattcctattgacaactcttctgggagtgctgttctgcttt

SEQ ID NO: 24 - DNA, 75 bp, Artificial Sequence
  IL-2Rb (TM)
attccgtggctcggccacctcctcgtgggtctcagcggggcttttggcttcatcatcttagtgtacttgctgatc

SEQ ID NO: 25 - DNA, 63 bp, Artificial Sequence
  IL-2RG (TM)
gtggttatctctgttggctccatgggattgattatcagccttctctgtgtgtatttctggctg

SEQ ID NO: 26 - protein, 286 aa, Artificial Sequence
  IL-2Rb (ICD, G2R-1)
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 27 - protein, 86 aa, Artificial Sequence
  IL-2RG (ICD, G2R-1)
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 28 - protein, 101 aa, Artificial Sequence
  gp130 (ICD, G2R-2)
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSIS

SEQ ID NO: 29 - protein, 205 aa, Artificial Sequence; identical to SEQ ID NO: 5
  IL-2Rb (ICD, G2R-2)

SEQ ID NO: 30 - protein, 62 aa, Artificial Sequence
  G-CSFR (ICD, G2R-3)
SPNRKNPLWPSVPDPAHSSLGSWVPTIMEEDAFQLPGLGTPPITKLTVLEEDEKKPVPWESH

SEQ ID NO: 31 - protein, 202 aa, Artificial Sequence
  IL-2Rb (ICD, G2R-3)
SSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 32 - protein, 67 aa, Artificial Sequence
  G-CSFR (ICD, G12R-1)
SPNRKNPLWPSVPDPAHSSLGSWVPTIMEEDAFQLPGLGTPPITKLTVLEEDEKKPVPWESHNSSET

SEQ ID NO: 33 - protein, 72 aa, Artificial Sequence
  IL-12Rb2 (ICD, G12R-1)
AGDLPTHDGYLPSNIDDLPSHEAPLADSLEELEPQHISLSVFPSSSLHPLTFSCGDKLTLDQLKMRCDSLML

SEQ ID NO: 34 - protein, 67 aa, Artificial Sequence; identical to SEQ ID NO: 32
  G-CSFR (ICD, G21R-1)

SEQ ID NO: 35 - protein, 30 aa, Artificial Sequence
  IL-21R (ICD, G21R-1)
SPGDEGPPRSYLRQWVVIPPPLSSPGPQAS

SEQ ID NO: 36 - protein, 63 aa, Artificial Sequence
  G-CSFR (ICD, G21R-2)
SPNRKNPLWPSVPDPAHSSLGSWVPTIMEEDAFQLPGLGTPPITKLTVLEEDEKKPVPWESHN

SEQ ID NO: 37 - protein, 183 aa, Artificial Sequence
  IL-21R (ICD, G21R-2)
QNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 38 - protein, 93 aa, Artificial Sequence
  G-CSFR (ICD, G21/2R-1)
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQ

SEQ ID NO: 39 - protein, 202 aa, Artificial Sequence; identical to SEQ ID NO: 31
  IL-2Rb (ICD, G21/2R-1)

SEQ ID NO: 40 - protein, 62 aa, Artificial Sequence; identical to SEQ ID NO: 30
  G-CSFR (ICD, G12/2R-1)

SEQ ID NO: 41 - protein, 43 aa, Artificial Sequence
  IL-2Rb (ICD, G12/2R-1)
SSNHSLTSCFTNQGYFFFHLPDALEIEACQVYFTYDPYSEEDP

SEQ ID NO: 42 - protein, 20 aa, Artificial Sequence
  IL-12Rb2 (ICD, G12/2R-1)
AGDLPTHDGYLPSNIDDLPS

SEQ ID NO: 43 - protein, 116 aa, Artificial Sequence
  IL-2Rb  (ICD, G12/2R-1)
GGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 44 - protein, 93 aa, Artificial Sequence; identical to SEQ ID NO: 38
  G-CSFR (ICD, G21/12/2R-1)

SEQ ID NO: 45 - protein, 43 aa, Artificial Sequence; identical to SEQ ID NO: 41
  IL-2Rb (ICD, G21/12/2R-1)

SEQ ID NO: 46 - protein, 20 aa, Artificial Sequence
  IL-12Rb2 (ICD, G21/12/2R-1)
AGDLPTHDGYLPSNIDDLPS

SEQ ID NO: 47 - protein, 116 aa, Artificial Sequence; identical to SEQ ID NO: 43
  IL-2Rb (ICD, G21/12/2R-1)

SEQ ID NO: 48 - protein, 138 aa, Artificial Sequence
  gp130 (ICD, G27/2R-1)
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSR

SEQ ID NO: 49 - protein, 205 aa, Artificial Sequence; identical to SEQ ID NO: 5
  IL-2Rb (ICD, G27/2R-1)

SEQ ID NO: 50 - protein, 62 aa, Artificial Sequence; identical to SEQ ID NO: 30
  G-CSFR (ICD, G7R-1)

SEQ ID NO: 51 - protein, 67 aa, Artificial Sequence
  IL-7Ra (ICD, G7R-1)
SGKNGPHVYQDLLLSLGTTNSTLPPPFSLQSGILTLNPVAQGQPILTSLGSNQEEAYVTMSSFYQNQ

SEQ ID NO: 52 - protein, 93 aa, Artificial Sequence; identical to SEQ ID NO: 38
  G-CSFR (ICD, G21/7R-1)

SEQ ID NO: 53 - protein, 77 aa, Artificial Sequence
  IL7R (ICD, G21/7R-1)
SSSRSLDCRESGKNGPHVYQDLLLSLGTTNSTLPPPFSLQSGILTLNPVAQGQPILTSLGSNQEEAYVTMSSFYQNQ

SEQ ID NO: 54 - DNA, 861 bp, Artificial Sequence
  IL-2RB (ICD, G2R-1)
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 55 - DNA, 261 bp, Artificial Sequence
  IL-2RG (ICD, G2R-1)
gaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 56 - DNA, 303 bp, Artificial Sequence
  gp130 (ICD, G2R-2)
aataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgtatgtcatcttctaggccaagcatttct

SEQ ID NO: 57 - DNA, 618 bp, Artificial Sequence
  IL-2Rb (ICD, G2R-2)
gcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 58 - DNA, 186 bp, Artificial Sequence
  G-CSFR (ICD, G2R-3)
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccat

SEQ ID NO: 59 - DNA, 609 bp, Artificial Sequence
  IL-2Rb (ICD, G2R-3)
agcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 60 - DNA, 201 bp, Artificial Sequence
  G-CSFR (ICD, G12R-1)
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacc

SEQ ID NO: 61 - DNA, 219 bp, Artificial Sequence
  IL-12Rb2 (ICD, G12R-1)
gcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 62 - DNA, 201 bp, Artificial Sequence; identical to SEQ ID NO: 60
  G-CSFR (ICD, G21R-1)

SEQ ID NO: 63 - DNA, 93 bp, Artificial Sequence
  IL-21R (ICD, G21R-1)
agccctggggacgaaggacccccccggagctacctccgccagtgggtggtcattcctccgccactttcgagccctggaccccaggccagctaa

SEQ ID NO: 64 - DNA, 189 bp, Artificial Sequence
  G-CSFR (ICD, G21R-2)
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataac

SEQ ID NO: 65 - DNA, 549 bp, Artificial Sequence
  IL-21R (ICD, G21R-2)
cagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccctggggacgaaggacccccccggagctacctccgccagtgggtggtcattcctccgccactttcgagccctggaccccaggccagc

SEQ ID NO: 66 - DNA, 279 bp, Artificial Sequence
  G-CSFR (ICD, G21/2R-1)
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccag

SEQ ID NO: 67 - DNA, 609 bp, Artificial Sequence; identical to SEQ ID NO: 59
  IL-2Rb (ICD, G21/2R-1)

SEQ ID NO: 68 - DNA, 186 bp, Artificial Sequence; identical to SEQ ID NO: 58
  G-CSFR (ICD, G12/2R-1)

SEQ ID NO: 69 - DNA, 129 bp, Artificial Sequence
  IL-2Rb (ICD, G12/2R-1)
agcagcaaccactcgctgaccagctgcttcaccaaccagggttacttcttcttccacctcccggatgccttggagatagaggcctgccaggtgtactttacttacgacccctactcagaggaagaccct

SEQ ID NO: 70 - DNA, 60 bp, Artificial Sequence
  IL-12Rb2 (ICD, G12/2R-1)
gcaggtgaccttcccacccatgatggctacttaccctccaacatagatgacctcccctca

SEQ ID NO: 71 - DNA, 351 bp, Artificial Sequence
  IL-2Rb (ICD, G12/2R-1)
ggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 72 - DNA, 279 bp, Artificial Sequence; identical to SEQ ID NO: 66
  G-CSFR (ICD, G21/12/2R-1)

SEQ ID NO: 73 - DNA, 129 bp, Artificial Sequence; identical to SEQ ID NO: 69
  IL-2Rb (ICD, G21/12/2R-1)

SEQ ID NO: 74 - DNA, 60 bp, Artificial Sequence; identical to SEQ ID NO: 70
  IL-12Rb2 (ICD, G21/12/2R-1)

SEQ ID NO: 75 - DNA, 351 bp, Artificial Sequence; identical to SEQ ID NO: 71
  IL-2Rb (ICD, G21/12/2R-1)

SEQ ID NO: 76 - DNA, 414 bp, Artificial Sequence
  gp130 (ICD, G27/2R-1)
aataagcgagacctaattaaaaaacacatctggcctaatgttccagatccttcaaagagtcatattgcccagtggtcacctcacactcctccaaggcacaatttcaattcaaaggatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgtatgtcatcttctaggccaagcatttctagcagtgatgaaaatgaatcttcacaaaacacttcgagcactgtccagtattctaccgtggtacacagtggctacagacaccaagttccgtcagtccaagtcttctcaaga

SEQ ID NO: 77 - DNA, 618 bp, Artificial Sequence; identical to SEQ ID NO: 57
  IL-2Rb (ICD, G27/2R-1)

SEQ ID NO: 78 - DNA, 186 bp, Artificial Sequence; identical to SEQ ID NO: 58
  G-CSFR (ICD, G7R-1)

SEQ ID NO: 79 - DNA, 204 bp, Artificial Sequence
  IL-7Ra (ICD, G7R-1)
agtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 80 - DNA, 279 bp, Artificial Sequence; identical to SEQ ID NO: 66
  G-CSFR (ICD, G21/7R-1)

SEQ ID NO: 81 - DNA, 234 bp, Artificial Sequence
  IL7R (ICD, G21/7R-1)
tcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 82 - protein, 175 aa, Artificial Sequence
  WT G-CSF
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 83 - protein, 175 aa, Artificial Sequence
  130a1 variant with mutation
MTPLGPASSLPQSFLLKCLEQVRKIQGDGAALQEKLCATYKLCHPERLVLLGHSLGIPWAPLSSCPSQALQLAGCLSQLHSGLFLYQGLLQALEGISPELGPTLDTLQKDVARFATTIWQQMKELGMAPALQPTQGAMPAFASAFQRRAGGVLVASHLQSFLEVSYRVLRHLAQP

SEQ ID NO: 84 - protein, 175 aa, Artificial Sequence
  307 variant with mutation
MTPLGPASSLPQEFLLDCLKQVRKIQGDGAALQEKLCATYKLCHPERLVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 85 - protein, 307 aa, Artificial Sequence
  WT G-CSFR
ECGHISVSAPIVHLGDPITASCIIKQNCSHLDPEPQILWRLGAELQPGGRQQRLSDGTQESIITLPHLNHTQAFLSCSLNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTE

SEQ ID NO: 86 - protein, 307 aa, Artificial Sequence
  134 G-CSFR variant
ECGHISVSAPIVHLGDPITASCIIKQNCSHLDPEPQILWELGAELQPGGRQQRLSDGTQESIITLPHLNHTQAFLSCSLNWGNSLQILDQVELRAGYPPAIPHNLSCLMNLTTSSLICQWEPGPETHLPTSFTLKSFKSEGNCQTQGDSILDCVPKDGQSHCSIPDKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTE

SEQ ID NO: 87 - protein, 307 aa, Artificial Sequence
  307 G-CSFR variant
ECGHISVSAPIVHLGDPITASCIIKQNCSHLDPEPQILWELGAELQPGGRQQRLSDGTQESIITLPHLNHTQAFLSCSLNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLKPMKVVKLEPPMLRTMDPSPEAAPPQAGCLQLSWEPWQPGLHINQKCELRHKPQRGEASWALVGPLPLEALQYELCGLLPATAYTLQIRCIEWPLPGHWSDWSPSLELRTTE

SEQ ID NO: 88 - protein, 23 aa, Artificial Sequence
  WT G-CSFR
IILGLFGLLLLLTCLCGTAWLCC

SEQ ID NO: 89 - protein, 24 aa, Artificial Sequence
  WT G-CSFR
MARLGNCSLTWAALIILLLPGSLE

SEQ ID NO: 90 - protein, 62 aa, Artificial Sequence; identical to SEQ ID NO: 30
  G2R-3 (G-CSFR)

SEQ ID NO: 91 - protein, 202 aa, Artificial Sequence; identical to SEQ ID NO: 31
  G2R-3 (IL-2R beta)

SEQ ID NO: 92 - protein, 505 aa, Artificial Sequence
  G4R (IL4R alfa)
DEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 93 - protein, 277 aa, Artificial Sequence
  G6R (gp130)
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 94 - protein, 235 aa, Artificial Sequence
  GEPOR
HRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 95 - protein, 65 aa, Artificial Sequence
  GIFNAR2 (G-CSFR)
SPNRKNPLWPSVPDPAHSSLGSWVPTIMEEDAFQLPGLGTPPITKLTVLEEDEKKPVPWESHNSS

SEQ ID NO: 96 - protein, 206 aa, Artificial Sequence
  GIFNAR2 (IFNAR2)
KKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 97 - protein, 66 aa, Artificial Sequence
  GIFNGR1 (IFN Gamma R2)
KYRGLIKYWFHTPPSIPLQIEEYLKDPTQPILEALDKDSSPKDDVWDSVSIISFPEKEQEDVLQTL

SEQ ID NO: 98 - protein, 31 aa, Artificial Sequence
  GIFNGR1 (IFN Gamma R1)
EGQELITVIKAPTSFGYDKPHVLVDLLVDDS

SEQ ID NO: 99 - protein, 64 aa, Artificial Sequence
  GIFNGR2 (G-CSFR)
SPNRKNPLWPSVPDPAHSSLGSWVPTIMEEDAFQLPGLGTPPITKLTVLEEDEKKPVPWESHNS

SEQ ID NO: 100 - protein, 32 aa, Artificial Sequence
  GIFNGR2 (IFN Gamma R1)
TEGQELITVIKAPTSFGYDKPHVLVDLLVDDS

SEQ ID NO: 101 - protein, 307 aa, Artificial Sequence; identical to SEQ ID NO: 85
  G-CSFR(Ig-CRH)

SEQ ID NO: 102 - protein, 193 aa, Artificial Sequence
  hIL-18
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 103 - protein, 109 aa, Artificial Sequence
  hCXCL13
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 104 - protein, 162 aa, Artificial Sequence
  hIL-21
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 105 - protein, 92 aa, Artificial Sequence
  hCCL3
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 106 - protein, 92 aa, Artificial Sequence
  hCCL4
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 107 - protein, 98 aa, Artificial Sequence
  hCCL19
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 108 - protein, 166 aa, Artificial Sequence
  hIFN Gamma
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 109 - protein, 189 aa, Artificial Sequence
  hIFN alfa -1/13
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 110 - protein, 155 aa, Artificial Sequence
  hIL-17
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 111 - protein, 551 aa, Artificial Sequence
  IL-2R beta/IL-15R beta
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 112 - protein, 369 aa, Artificial Sequence
  IL-2R Gamma c
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 113 - protein, 825 aa, Artificial Sequence
  IL-4R alfa
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 114 - protein, 521 aa, Artificial Sequence
  IL-9R
MGLGRCIWEGWTLESEALRRDMGTWLLACICICTCVCLGVSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 115 - protein, 862 aa, Artificial Sequence
  IL-12R breta2
MAHTFRGCSLAFMFIITWLLIKAKIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 116 - protein, 538 aa, Artificial Sequence
  IL-21R
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 117 - protein, 918 aa, Artificial Sequence
  gp130
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 118 - protein, 21 aa, Artificial Sequence
  IL-7R alfa
KRIKPIVWPSLPDHKKTLEHL

SEQ ID NO: 119 - protein, 16 aa, Artificial Sequence
  IL-7R alfa
KKPRKNLNVSFNPESF

SEQ ID NO: 120 - protein, 16 aa, Artificial Sequence
  IL-7R alfa
PESFLDCQIHRVDDIQ

SEQ ID NO: 121 - protein, 21 aa, Artificial Sequence
  IL-2R beta
PWLKKVLKCNTPDPSKFFSQL

SEQ ID NO: 122 - protein, 16 aa, Artificial Sequence
  IL-2R beta
APEISPLEVLERDKVT

SEQ ID NO: 123 - protein, 17 aa, Artificial Sequence
  IL-2R beta
TSCFTNQGYFFFHLPDA

SEQ ID NO: 124 - protein, 17 aa, Artificial Sequence
  IL-2R beta
RLPLNTDAYLSLQELQG

SEQ ID NO: 125 - protein, 16 aa, Artificial Sequence
  IL-2R Gamma c
ERTMPRIPTLKNLEDL

SEQ ID NO: 126 - protein, 16 aa, Artificial Sequence
  IL-2R Gamma c
PDYSERLCLVSEIPPK

SEQ ID NO: 127 - protein, 20 aa, Artificial Sequence
  IL-4R alfa
KIKKEWWDQIPNPARSRLVA

SEQ ID NO: 128 - protein, 16 aa, Artificial Sequence
  IL-4R alfa
LTKLLPCFLEHNMKRD

SEQ ID NO: 129 - protein, 17 aa, Artificial Sequence
  IL-4R alfa
LVIAGNPAYRSFSNSLS

SEQ ID NO: 130 - protein, 17 aa, Artificial Sequence
  IL-4R alfa
LGPPGEAGYKAFSSLLA

SEQ ID NO: 131 - protein, 17 aa, Artificial Sequence
  IL-4R alfa
GASSGEEGYKPFQDLIP

SEQ ID NO: 132 - protein, 21 aa, Artificial Sequence
  IL-9R
SPRVKRIFYQNVPSPAMFFQP

SEQ ID NO: 133 - protein, 16 aa, Artificial Sequence
  IL-9R
GAHGAGVLLSQDCAGT

SEQ ID NO: 134 - protein, 16 aa, Artificial Sequence
  IL-9R
GTPQGALEPCVQEATA

SEQ ID NO: 135 - protein, 26 aa, Artificial Sequence
  IL-12R beta2
AGDLPTHDGYLPSNIDDLPSHEAPLA

SEQ ID NO: 136 - protein, 21 aa, Artificial Sequence
  IL-21R
LWRLWKKIWAVPSPERFFMPL

SEQ ID NO: 137 - protein, 16 aa, Artificial Sequence
  IL-21R
PFTGSSLELGPWSPEV

SEQ ID NO: 138 - protein, 16 aa, Artificial Sequence
  IL-21R
PEVPSTLEVYSCHPPR

SEQ ID NO: 139 - protein, 17 aa, Artificial Sequence
  IL-21R
GDEGPPRSYLRQWVVIP

SEQ ID NO: 140 - protein, 21 aa, Artificial Sequence
  gp130
DLIKKHIWPNVPDPSKSHIAQ

SEQ ID NO: 141 - protein, 16 aa, Artificial Sequence
  gp130
DGNFTDVSVVEIEAND

SEQ ID NO: 142 - protein, 16 aa, Artificial Sequence
  gp130
QNTSSTVQYSTVVHSG

SEQ ID NO: 143 - protein, 16 aa, Artificial Sequence
  gp130
STVVHSGYRHQVPSVQ

SEQ ID NO: 144 - protein, 20 aa, Artificial Sequence
  gp130
DGILPRQQYFKQNCSQHESS

SEQ ID NO: 145 - protein, 14 aa, Artificial Sequence
  gp130
EGMPKSYLPQTVRQ

SEQ ID NO: 146 - protein, 10 aa, Artificial Sequence
  gp130
TVRQGGYMPQ

SEQ ID NO: 147 - protein, 598 aa, Artificial Sequence
  HPV 16 E7eTCR (LVL-modified chimeric TCR)
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 148 - protein, 598 aa, Artificial Sequence
  HPV 16 E7eTCR (Cys-substituted, LVL-modified chimeric TCR)
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 149 - protein, 274 aa, Artificial Sequence
  NY-ESO-1eTCR (NY-ESO-1c259 TCR a chain)
  MISC_FEATURE (136)..(136): Xaa = Pyrrolysine
  MISC_FEATURE (144)..(144): Xaa = Pyrrolysine
  MISC_FEATURE (164)..(164): Xaa = Pyrrolysine
  MISC_FEATURE (169)..(169): Xaa = Pyrrolysine
METLLGLLILWLGLGWVSSKGEVTGIPAALSVPEGENLVLNCSFTDSAIYNLGWFRGDPGKGLTSLLLIGSSGREGTSGRLNASLDKSSGRSTLYIAASGPGDSATYLCAVRPLYGGSYIPTFGRGTSLIVHPYIXNPDPAVYXLRDSKSSDKSVCLFTDFDSXTNVSXSKDSDVYITDKTVLDMRSMDFKSNSAVAWSNKSDFACANAFNNSIIPEDTFFPSPESSCDVKLVEKSFETDTNLNFCNLSVIGFRILLLKVAGFNLLMTLRLWSS

SEQ ID NO: 150 - protein, 312 aa, Artificial Sequence
  NY-ESO-1eTCR (NY-ESO-1 c259 TCR b chain)
  MISC_FEATURE (244)..(244): Xaa = Pyrrolysine
  MISC_FEATURE (252)..(252): Xaa = Pyrrolysine
  MISC_FEATURE (272)..(272): Xaa = Pyrrolysine
  MISC_FEATURE (273)..(273): Xaa = Pyrrolysine
RMSIGLLCCAALSLLWAGPVNAGVTGTPKFGVLKTGGSMTLGCAGDMNHEYMSWYRGDPGMGLRLIHYSVGAGITDGGEVPNGYNVSRSTTEDFPLRLLSAAPSGTSVYFCASSYVGNTGELFFGEGSRLTVLEDLKNVFPPEVAVFEPSEAEISHTCKATLVCLATGFYPDHVELSWWVNGKEVHSGVSTDPGPLKEGPALNDSRYCLSSRLRVSATFWGNPRNHFRCGVGFYGLSENDEWTXDRAKPVTXIVSAEAWGRADCGFTSESYXXGVLSATILYEILLGKATLYAVLVSALVLMAMVKRKDSRG

SEQ ID NO: 151 - protein, 486 aa, Artificial Sequence
  Second generation 4-1BB containing anti-CD19 CAR (CTL019)
MALPVTALLLPLALLLHAARPDIQMTQTTSSLSASLGDRVTISCRASQDISKYLNWYQQKPDGTVKLLIYHTSRIHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRITIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 21 aa, Artificial Sequence
  Second generation 4-1BB containing anti-CD19 CAR (Leader)
MALPVTALLLPLALLLHAARP

SEQ ID NO: 153 - protein, 107 aa, Artificial Sequence
  Second generation 4-1BB containing anti-CD19 CAR (VH)
DIQMTQTTSSLSASLGDRVTISCRASQDISKYLNWYQQKPDGTVKLLIYHTSRIHSGVPSRFSGSGSGTDYSLTISNLEQEDIATYFCQQGNTLPYTFGGGTKLEIT

SEQ ID NO: 154 - protein, 15 aa, Artificial Sequence
  Second generation 4-1BB containing anti-CD19 CAR (Linker)
GGGGSGGGGSGGGGS

SEQ ID NO: 155 - protein, 120 aa, Artificial Sequence
  Second generation 4-1BB containing anti-CD19 CAR (VL)
EVKLQESGPGLVAPSQSLSVTCTVSGVSLPDYGVSWIRQPPRKGLEWLGVIWGSETTYYNSALKSRITIIKDNSKSQVFLKMNSLQTDDTAIYYCAKHYYYGGSYAMDYWGQGTSVTVSS

SEQ ID NO: 156 - protein, 45 aa, Artificial Sequence
  Second generation 4-1BB containing anti-CD19 CAR (CD8 hinge)
TTTPAPRPPTPAPTIASQPLSLRPEACRPAAGGAVHTRGLDFACD

SEQ ID NO: 157 - protein, 24 aa, Artificial Sequence
  Second generation 4-1BB containing anti-CD19 CAR (CD8 TM)
IYIWAPLAGTCGVLLLSLVITLYC

SEQ ID NO: 158 - protein, 42 aa, Artificial Sequence
  Second generation 4-1BB containing anti-CD19 CAR (4-1BB)
KRGRKKLLYIFKQPFMRPVQTTQEEDGCSCRFPEEEEGGCEL

SEQ ID NO: 159 - protein, 112 aa, Artificial Sequence
  Second generation 4-1BB containing anti-CD19 CAR (CD3 Zeta)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 465 aa, Artificial Sequence
  First generation anti-mesothelin CAR (P4-z)
MALPVTALLLPLALLLHAARPGSQVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSASTTTPAPRPPTPAPTIASRPLSLRPEACRPAAGGAVHTRGLDFACDIYIWAPLAGTCGVLLLSLVITLYCRVKFSRSADAPAYQNGQNQLYNELNLGRREEYDVLDKRRGRDPEMGGKPRRKNPQEGLYNELNKDKMAEAYSEIGMKGERRRGKGHDGLYQGLSTATKDTYDALHMQALPPR

SEQ ID NO: 161 - protein, 34 aa, Artificial Sequence
  First generation anti-mesothelin CARb (Leader)
MALPVTALLLPLALLLHAARPGSQVQLQQSGPGL

SEQ ID NO: 162 - protein, 115 aa, Artificial Sequence
  First generation anti-mesothelin CARb (VH)
VTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGIL

SEQ ID NO: 163 - protein, 17 aa, Artificial Sequence
  First generation anti-mesothelin CARb (Linker)
GSGGGGSGGGGSGGGGS

SEQ ID NO: 164 - protein, 118 aa, Artificial Sequence
  First generation anti-mesothelin CARb (VL)
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSAS

SEQ ID NO: 165 - protein, 45 aa, Artificial Sequence
  First generation anti-mesothelin CARb (CD8 hinge)
TTTPAPRPPTPAPTIASRPLSLRPEACRPAAGGAVHTRGLDFACD

SEQ ID NO: 166 - protein, 24 aa, Artificial Sequence
  First generation anti-mesothelin CARb (CD8 TM)
IYIWAPLAGTCGVLLLSLVITLYC

SEQ ID NO: 167 - protein, 112 aa, Artificial Sequence
  First generation anti-mesothelin CARb (CD3 Zeta)
RVKFSRSADAPAYQNGQNQLYNELNLGRREEYDVLDKRRGRDPEMGGKPRRKNPQEGLYNELNKDKMAEAYSEIGMKGERRRGKGHDGLYQGLSTATKDTYDALHMQALPPR

SEQ ID NO: 168 - protein, 490 aa, Artificial Sequence
  Second generation CD28 containing anti-mesothelin CAR (HN1scFv-28Z)
MVLLVTSLLLCELPHPAFLLIPDIQAQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 25 aa, Artificial Sequence
  Second generation CD28 containing anti-mesothelin CAR (Leader)
MVLLVTSLLLCELPHPAFLLIPDIQ

SEQ ID NO: 170 - protein, 120 aa, Artificial Sequence
  Second generation CD28 containing anti-mesothelin CAR (VH)
AQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSS

SEQ ID NO: 171 - protein, 15 aa, Artificial Sequence
  Second generation CD28 containing anti-mesothelin CAR (Linker)
GGGGSGGGGSGGGGS

SEQ ID NO: 172 - protein, 108 aa, Artificial Sequence
  Second generation CD28 containing anti-mesothelin CAR (VL)
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKR

SEQ ID NO: 173 - protein, 42 aa, Artificial Sequence
  Second generation CD28 containing anti-mesothelin CAR (CD8 hinge)
AAAIEVMYPPPYLDNEKSNGTIIHVKGKHLCPSPLFPGPSKP

SEQ ID NO: 174 - protein, 27 aa, Artificial Sequence
  Second generation CD28 containing anti-mesothelin CAR (CD8 TM)
FWVLVVVGGVLACYSLLVTVAFIIFWV

SEQ ID NO: 175 - protein, 41 aa, Artificial Sequence
  Second generation CD28 containing anti-mesothelin CAR (CD28 ICD)
RSKRSRLLHSDYMNMTPRRPGPTRKHYQPYAPPRDFAAYRS

SEQ ID NO: 176 - protein, 112 aa, Artificial Sequence
  Second generation CD28 containing anti-mesothelin CAR (CD3 zeta)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 177 - protein, 554 aa, Artificial Sequence
  Third generation CD28 and 4-1BB containing anti-mesothelin CAR (HN1scFv-28BBZ)
MVLLVTSLLLCELPHPAFLLIPDIQAQVQLVQSGAEVKRPGASVQVSCRASGYSINTYYMQWVRQAPGAGLEWMGVINPSGVTSYAQKFQGRVTLTNDTSTNTVYMQLNSLTSADTAVYYCARWALWGDFGMDVWGKGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - protein, 25 aa, Artificial Sequence
  Third generation CD28 and 4-1BB containing anti-mesothelin CAR (Leader)
MVLLVTSLLLCELPHPAFLLIPDIQ

SEQ ID NO: 179 - protein, 120 aa, Artificial Sequence; identical to SEQ ID NO: 170
  Third generation CD28 and 4-1BB containing anti-mesothelin CAR (VH)

SEQ ID NO: 180 - protein, 15 aa, Artificial Sequence
  Third generation CD28 and 4-1BB containing anti-mesothelin CAR (Linker)
GGGGSGGGGSGGGGS

SEQ ID NO: 181 - protein, 108 aa, Artificial Sequence; identical to SEQ ID NO: 172
  Third generation CD28 and 4-1BB containing anti-mesothelin CAR (VL)

SEQ ID NO: 182 - protein, 58 aa, Artificial Sequence
  Third generation CD28 and 4-1BB containing anti-mesothelin CAR (CD28 hinge)
AAAFVPVFLPAKPTTTPAPRPPTPAPTIASQPLSLRPEACRPAAGGAVHTRGLDFACD

SEQ ID NO: 183 - protein, 28 aa, Artificial Sequence
  Third generation CD28 and 4-1BB containing anti-mesothelin CAR (CD28 TM)
IYIWAPLAGTCGVLLLSLVITLYCNHRN

SEQ ID NO: 184 - protein, 41 aa, Artificial Sequence; identical to SEQ ID NO: 175
  Third generation CD28 and 4-1BB containing anti-mesothelin CAR (CD28 ICD)

SEQ ID NO: 185 - protein, 47 aa, Artificial Sequence
  Third generation CD28 and 4-1BB containing anti-mesothelin CAR (4-1BB)
RFSVVKRGRKKLLYIFKQPFMRPVQTTQEEDGCSCRFPEEEEGGCEL

SEQ ID NO: 186 - protein, 112 aa, Artificial Sequence; identical to SEQ ID NO: 176
  Third generation CD28 and 4-1BB containing anti-mesothelin CAR (CD3 zeta)

SEQ ID NO: 187 - protein, 219 aa, Artificial Sequence
  IL-7R alfa
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 188 - protein, 25 aa, Artificial Sequence
  IL-7R alfa
PILLTISILSFFSVALLVILACVLW

SEQ ID NO: 189 - protein, 195 aa, Artificial Sequence
  IL-7R alfa
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 190 - protein, 20 aa, Artificial Sequence
  IL-7R alfa
MTILGTTFGMVFSLLQVVSG

SEQ ID NO: 191 - protein, 152 aa, Artificial Sequence
  IL-7
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 192 - protein, 286 aa, Artificial Sequence; identical to SEQ ID NO: 26
  7/2R-1 (IL-2R?)

SEQ ID NO: 193 - protein, 58 aa, Artificial Sequence
  7/2R-2 (IL-7R alfa)
KKRIKPIVWPSLPDHKKTLEHLCKKPRKNLNVSFNPESFLDCQIHRVDDIQARDEVEG

SEQ ID NO: 194 - protein, 224 aa, Artificial Sequence
  7/2R-2 (IL-2R beta)
LERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 195 - protein, 76 aa, Artificial Sequence
  7/2/12R-1 (IL-12R beta2)
TVLPAGDLPTHDGYLPSNIDDLPSHEAPLADSLEELEPQHISLSVFPSSSLHPLTFSCGDKLTLDQLKMRCDSLML

SEQ ID NO: 196 - protein, 127 aa, Artificial Sequence
  7/12/2R-1 (IL-2R beta)
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDP

SEQ ID NO: 197 - protein, 20 aa, Artificial Sequence
  7/12/2R-1 (IL-12R beta2)
AGDLPTHDGYLPSNIDDLPS

SEQ ID NO: 198 - protein, 116 aa, Artificial Sequence; identical to SEQ ID NO: 43
  7/12/2R-1 (IL-2R beta)

SEQ ID NO: 199 - protein, 53 aa, Artificial Sequence
  7/12/2R-2 (IL-7R alfa)
KKRIKPIVWPSLPDHKKTLEHLCKKPRKNLNVSFNPESFLDCQIHRVDDIQAR

SEQ ID NO: 200 - protein, 67 aa, Artificial Sequence
  7/12/2R-2 (IL-12R beta2)
AGDLPTHDGYLPSNIDDLPSHEAPLADSLEELEPQHISLSVFPSSSLHPLTFSCGDKLTLDQLKMRC

SEQ ID NO: 201 - protein, 279 aa, Artificial Sequence
  7/21-R1 (IL-21R)
LWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 202 - protein, 54 aa, Artificial Sequence
  7/21-R2 (IL-7R alfa)
KKRIKPIVWPSLPDHKKTLEHLCKKPRKNLNVSFNPESFLDCQIHRVDDIQARD

SEQ ID NO: 203 - protein, 216 aa, Artificial Sequence
  7/21-R2 (7/21-R2)
RSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 204 - protein, 71 aa, Artificial Sequence
  7/21/7R-1 (IL-7R alfa)
DCRESGKNGPHVYQDLLLSLGTTNSTLPPPFSLQSGILTLNPVAQGQPILTSLGSNQEEAYVTMSSFYQNQ

SEQ ID NO: 205 - protein, 195 aa, Artificial Sequence; identical to SEQ ID NO: 189
  7/7/21R-1 (IL-7R alfa)

SEQ ID NO: 206 - protein, 30 aa, Artificial Sequence; identical to SEQ ID NO: 35
  7/7/21R-1 (IL-21R)

SEQ ID NO: 207 - protein, 29 aa, Artificial Sequence
  7/2/21/12R-1 (IL-21R)
SPGDEGPPRSYLRQWVVIPPPLSSPGPQA

SEQ ID NO: 208 - protein, 72 aa, Artificial Sequence; identical to SEQ ID NO: 33
  7/2/21/12R-1 (IL-12R beta2)

SEQ ID NO: 209 - protein, 569 aa, Artificial Sequence
  7/4R-1 (IL-4R alfa)
KIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 210 - protein, 52 aa, Artificial Sequence
  7/4R-2 (IL-7R alfa)
KKRIKPIVWPSLPDHKKTLEHLCKKPRKNLNVSFNPESFLDCQIHRVDDIQA

SEQ ID NO: 211 - protein, 505 aa, Artificial Sequence; identical to SEQ ID NO: 92
  7/4R-2 (IL-4R alfa)

SEQ ID NO: 212 - protein, 278 aa, Artificial Sequence
  7/6R-1 (Gp130)
KNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 213 - protein, 57 aa, Artificial Sequence
  7/6R-2 (IL-7R alfa)
KKRIKPIVWPSLPDHKKTLEHLCKKPRKNLNVSFNPESFLDCQIHRVDDIQARDEVE

SEQ ID NO: 214 - protein, 218 aa, Artificial Sequence
  7/6R-2 (Gp130)
KKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 215 - protein, 233 aa, Artificial Sequence
  hTNF- alfa
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 216 - protein, 216 aa, Artificial Sequence
  hNKG2-D: ICD-TM-ECD
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 217 - DNA, 4758 bp, Artificial Sequence
  CAR_P2A_G12-2R1-134_T2A_hIL-18
cccgccaggatcctgacgcgtatggtgctgctggtcaccagcttactcctgtgcgagctcccccatcctgcgttcttgctgatccccgacatccagcaggtgcagctgcaacagtcgggccccgagttagaaaaacccggcgcctccgtgaagatctcgtgtaaagcttcgggctactcgtttaccggctacaccatgaattgggttaagcagagccatggaaagtctcttgaatggataggcctgattacgccttataatggggcctcgagctacaaccagaaatttcgtggtaaggcaactctgactgtagacaagtcgtcctccaccgcatacatggatcttctgagcctaacctcagaagacagcgcagtctacttctgcgcccgtggtggttacgatggccgcggattcgactattggggccagggcaccaccgtcaccgtgagctccggcggtggcgggtccggtggcggcggctcaagcggaggaggtagtgacatcgagttgacccagtcgcctgccatcatgtccgcatcccccggcgagaaggtgacgatgacgtgttctgcttccagctctgtatcgtacatgcactggtaccaacagaagtccggcacctcccccaagcgttggatctacgacacttccaagctggcgtctggcgtccctgggcgcttttcaggtagcgggtccggcaatagctattccttgacgatcagtagcgtggaggctgaggatgatgccacctactactgccagcagtggagcaagcacccgctcaccttcggcgccgggaccaagcttgaaatcaaggcggcggcgatcgaggtgatgtatcctcctccctacctggacaacgagaaatccaatggcactatcatccacgtgaaggggaagcacctgtgcccgagccccctgttccccggcccttccaagcctttttgggtgcttgttgtggtcggcggcgtgctggcctgctactctctgctggtaaccgtagcgttcatcatcttctgggtgcgatctaagcggtcccggctgttgcacagtgactacatgaacatgaccccgcgccgccccggcccaacccgcaagcactaccagccgtacgctccacctcgcgactttgccgcctacaggtcgcgagtgaaattttcgcgctccgcggacgcgccggcctatcagcagggccagaaccagctttacaacgagttgaacctcgggcgcagggaggagtacgacgtgctggataaacgtagaggtcgggacccggagatgggcggcaaaccacgccgcaagaaccctcaggaaggcctgtacaacgagctccagaaggacaaaatggctgaggcgtactcggagattgggatgaaaggcgagcggcgccgcggcaagggccacgatggcctgtaccaggggctatctaccgctacaaaggacacctacgacgccctacatatgcaggccctgccaccgcgtggctctggcgcaaccaacttctcgcttctgaagcaggccggtgatgtggaggaaaacccaggtcctatggcccgcctgggcaactgctcgctgacctgggcggccctgatcatcctgctgctgcccggctctctggaggagtgtggccatatttccgtgtcagctccaatcgtgcacctgggcgaccccatcaccgcttcttgcatcatcaagcagaactgctctcacctcgaccctgagccccaaatactttgggagctgggcgcggagctccagcctggaggacgccagcagcgtttgtcagacggcactcaggaatccatcatcaccctcccacatctgaatcacacccaggcgttcctgtcttgttgtctcaactggggcaactccttgcagatactggaccaggtggagctgagagctggctatcccccggcaattcctcacaacctgtcctgtctgatgaacctgaccacctccagtctgatctgtcagtgggagcccgggccagagactcaccttcccaccagcttcacccttaagtcgttcaaatccgagggcaactgccagacccagggtgattctattttggattgcgtgcccaaggacgggcagtctcactgttgtattcccgacaagcacctccttttataccagaatatgggcatctgggtgcaggccgagaatgccctgggaacgtctatgtctccacagctatgcctggatccaatggacgtagtgaagctggagccccccatgctacgcactatggatcctagcccagaggccgctccccctcaggcggggtgcctgcagctttgttgggaaccctggcaacccgggttgcacattaaccagaagtgcgagctgcgccacaagccccagagaggagaggcctcctgggccctcgtgggccctctgccgttggaggccttgcagtatgagctgtgcggcctcttgcccgctacagcttatactctgcaaatcaggtgtattcgctggcctttgcccggacactggtccgactggtcccccagccttgagctgagaaccacggagcgcgcccccaccgtacgcttggacacctggtggcgccagaggcagctggaccctcgcactgtgcagctgttctggaagccggtgcccctggaggaggactccggacgcatccagggctacgtggtcagctggcgtccgagtgggcaggccggggccatcctgcccctgtgcaacacgactgagctgagctgcactttccatctgcccagcgaggctcaggaggtggcgctcgtggcttacaactccgcaggcacttctcgcccaacccccgtggtgttctctgagagtcggggccccgccctgacgcgccttcacgccatggcgcgcgacccgcattccctttgggtcggttgggagccacctaacccatggccgcagggctacgtgatcgagtggggcctgggccccccgagcgcatcgaacagcaacaagacctggagaatggaacagaacggccgcgcaaccggcttcctattaaaggagaacatccgcccgttccagctgtacgagatcatcgtgacaccgctgtaccaggacactatggggccttctcagcacgtttacgcctactcccaggagatggccccgagccacgcaccggaactgcacctaaagcacatcggtaagacctgggcacagctcgagtgggtccccgagcctcccgagctaggcaagtcgccgctgacccattacaccatcttctggaccaacgcgcagaaccagtcctttagcgcgatcctcaatgcctcctcccgcggtttcgtcttgcacgggctggagccagcctccttataccacatccacctgatggcggcttcccaggctggtgctaccaacagcacagttttgacactgatgaccctgacccccgagggctcggagctccacatcattttgggactcttcggactgctgctgctgctgacctgcctgtgcggcactgcttggctctgctgttctcccaaccgcaagaaccctctttggccctctgtccctgacccggcccattctagcctgggatcttgggtcccgaccatcatggaagaagacgcgttccagctccctgggctgggcacccctccgatcacgaagctcacggtcctggaggaggacgagaagaagcccgtaccgtgggagagccactcctcgaatcatagcctaacctcctgctttacaaaccaggggtatttttttttccacttgcccgacgctctggagatcgaggcctgccaggtgtacttcacctacgacccctacagcgaggaggaccctgccggtgatctgcccacccatgacggctatctgccatcaaacattgacgacctgccgtccggaggtcccagtcctccgtccaccgcccccggcggttctggtgccggcgaagaacggatgcctccctccctgcaggagcgtgtgccacgggattgggacccacagcccttagggccgcctacccccggcgtgcccgacctcgtcgatttccagccgcctcctgagctggttctccgagaggcaggagaggaggtgcctgatgctggaccccgtgagggcgtgagctttccttggtctcgcccgccaggacagggggagttccgtgctctcaacgcgcgtctgccgttgaacactgatgcatacctcagtcttcaggagctgcagggccaggatccaacccacctcgttggctcaggggaaggtcgcggctcgctgttgacgtgtggtgacgtggaggagaaccccggcccgtacttcggtaagctggagtctaaactgtcggtgatacggaacctgaacgaccaggtcctgttcattgaccagggtaaccgcccgctgttcgaggacatgactgactccgattgccgcgacaatgccccccgcacgatcttcattatctccatgtacaaggatagccaaccccgtggtatggccgtgaccattagcgtcaaatgtgagaagatctctactctgtcctgtgagaataagattatctccttcaaggagatgaacccccctgataacatcaaagacactaaatcggacatcattttctttcagaggagcgtgccaggccacgacaacaagatgcagttcgagagttctagttacgagggatatttcctggcctgcgaaaaagaacgtgacctcttcaagctaatcctgaagaaggaggacgagctcggtgacaggagcatcatgtttactgtccaaaacgaggactga

SEQ ID NO: 218 - DNA, 21 bp, Artificial Sequence
  5′ upstream sequence including Kozak
cccgccaggatcctgacgcgt

SEQ ID NO: 219 - DNA, 1461 bp, Artificial Sequence
  Mesothelin CAR
atggtgctgctggtcaccagcttactcctgtgcgagctcccccatcctgcgttcttgctgatccccgacatccagcaggtgcagctgcaacagtcgggccccgagttagaaaaacccggcgcctccgtgaagatctcgtgtaaagcttcgggctactcgtttaccggctacaccatgaattgggttaagcagagccatggaaagtctcttgaatggataggcctgattacgccttataatggggcctcgagctacaaccagaaatttcgtggtaaggcaactctgactgtagacaagtcgtcctccaccgcatacatggatcttctgagcctaacctcagaagacagcgcagtctacttctgcgcccgtggtggttacgatggccgcggattcgactattggggccagggcaccaccgtcaccgtgagctccggcggtggcgggtccggtggcggcggctcaagcggaggaggtagtgacatcgagttgacccagtcgcctgccatcatgtccgcatcccccggcgagaaggtgacgatgacgtgttctgcttccagctctgtatcgtacatgcactggtaccaacagaagtccggcacctcccccaagcgttggatctacgacacttccaagctggcgtctggcgtccctgggcgcttttcaggtagcgggtccggcaatagctattccttgacgatcagtagcgtggaggctgaggatgatgccacctactactgccagcagtggagcaagcacccgctcaccttcggcgccgggaccaagcttgaaatcaaggcggcggcgatcgaggtgatgtatcctcctccctacctggacaacgagaaatccaatggcactatcatccacgtgaaggggaagcacctgtgcccgagccccctgttccccggcccttccaagcctttttgggtgcttgttgtggtcggcggcgtgctggcctgctactctctgctggtaaccgtagcgttcatcatcttctgggtgcgatctaagcggtcccggctgttgcacagtgactacatgaacatgaccccgcgccgccccggcccaacccgcaagcactaccagccgtacgctccacctcgcgactttgccgcctacaggtcgcgagtgaaattttcgcgctccgcggacgcgccggcctatcagcagggccagaaccagctttacaacgagttgaacctcgggcgcagggaggagtacgacgtgctggataaacgtagaggtcgggacccggagatgggcggcaaaccacgccgcaagaaccctcaggaaggcctgtacaacgagctccagaaggacaaaatggctgaggcgtactcggagattgggatgaaaggcgagcggcgccgcggcaagggccacgatggcctgtaccaggggctatctaccgctacaaaggacacctacgacgccctacatatgcaggccctgccaccgcgt

SEQ ID NO: 220 - DNA, 66 bp, Artificial Sequence
  P2A
ggctctggcgcaaccaacttctcgcttctgaagcaggccggtgatgtggaggaaaacccaggtcct

SEQ ID NO: 221 - DNA, 2673 bp, Artificial Sequence
  G12/2R-1 134 ECD
atggcccgcctgggcaactgctcgctgacctgggcggccctgatcatcctgctgctgcccggctctctggaggagtgtggccatatttccgtgtcagctccaatcgtgcacctgggcgaccccatcaccgcttcttgcatcatcaagcagaactgctctcacctcgaccctgagccccaaatactttgggagctgggcgcggagctccagcctggaggacgccagcagcgtttgtcagacggcactcaggaatccatcatcaccctcccacatctgaatcacacccaggcgttcctgtcttgttgtctcaactggggcaactccttgcagatactggaccaggtggagctgagagctggctatcccccggcaattcctcacaacctgtcctgtctgatgaacctgaccacctccagtctgatctgtcagtgggagcccgggccagagactcaccttcccaccagcttcacccttaagtcgttcaaatccgagggcaactgccagacccagggtgattctattttggattgcgtgcccaaggacgggcagtctcactgttgtattcccgacaagcacctccttttataccagaatatgggcatctgggtgcaggccgagaatgccctgggaacgtctatgtctccacagctatgcctggatccaatggacgtagtgaagctggagccccccatgctacgcactatggatcctagcccagaggccgctccccctcaggcggggtgcctgcagctttgttgggaaccctggcaacccgggttgcacattaaccagaagtgcgagctgcgccacaagccccagagaggagaggcctcctgggccctcgtgggccctctgccgttggaggccttgcagtatgagctgtgcggcctcttgcccgctacagcttatactctgcaaatcaggtgtattcgctggcctttgcccggacactggtccgactggtcccccagccttgagctgagaaccacggagcgcgcccccaccgtacgcttggacacctggtggcgccagaggcagctggaccctcgcactgtgcagctgttctggaagccggtgcccctggaggaggactccggacgcatccagggctacgtggtcagctggcgtccgagtgggcaggccggggccatcctgcccctgtgcaacacgactgagctgagctgcactttccatctgcccagcgaggctcaggaggtggcgctcgtggcttacaactccgcaggcacttctcgcccaacccccgtggtgttctctgagagtcggggccccgccctgacgcgccttcacgccatggcgcgcgacccgcattccctttgggtcggttgggagccacctaacccatggccgcagggctacgtgatcgagtggggcctgggccccccgagcgcatcgaacagcaacaagacctggagaatggaacagaacggccgcgcaaccggcttcctattaaaggagaacatccgcccgttccagctgtacgagatcatcgtgacaccgctgtaccaggacactatggggccttctcagcacgtttacgcctactcccaggagatggccccgagccacgcaccggaactgcacctaaagcacatcggtaagacctgggcacagctcgagtgggtccccgagcctcccgagctaggcaagtcgccgctgacccattacaccatcttctggaccaacgcgcagaaccagtcctttagcgcgatcctcaatgcctcctcccgcggtttcgtcttgcacgggctggagccagcctccttataccacatccacctgatggcggcttcccaggctggtgctaccaacagcacagttttgacactgatgaccctgacccccgagggctcggagctccacatcattttgggactcttcggactgctgctgctgctgacctgcctgtgcggcactgcttggctctgctgttctcccaaccgcaagaaccctctttggccctctgtccctgacccggcccattctagcctgggatcttgggtcccgaccatcatggaagaagacgcgttccagctccctgggctgggcacccctccgatcacgaagctcacggtcctggaggaggacgagaagaagcccgtaccgtgggagagccactcctcgaatcatagcctaacctcctgctttacaaaccaggggtatttttttttccacttgcccgacgctctggagatcgaggcctgccaggtgtacttcacctacgacccctacagcgaggaggaccctgccggtgatctgcccacccatgacggctatctgccatcaaacattgacgacctgccgtccggaggtcccagtcctccgtccaccgcccccggcggttctggtgccggcgaagaacggatgcctccctccctgcaggagcgtgtgccacgggattgggacccacagcccttagggccgcctacccccggcgtgcccgacctcgtcgatttccagccgcctcctgagctggttctccgagaggcaggagaggaggtgcctgatgctggaccccgtgagggcgtgagctttccttggtctcgcccgccaggacagggggagttccgtgctctcaacgcgcgtctgccgttgaacactgatgcatacctcagtcttcaggagctgcagggccaggatccaacccacctcgtt

SEQ ID NO: 222 - DNA, 63 bp, Artificial Sequence
  T2A
ggctcaggggaaggtcgcggctcgctgttgacgtgtggtgacgtggaggagaaccccggcccg

SEQ ID NO: 223 - DNA, 474 bp, Artificial Sequence
  IL-18
tacttcggtaagctggagtctaaactgtcggtgatacggaacctgaacgaccaggtcctgttcattgaccagggtaaccgcccgctgttcgaggacatgactgactccgattgccgcgacaatgccccccgcacgatcttcattatctccatgtacaaggatagccaaccccgtggtatggccgtgaccattagcgtcaaatgtgagaagatctctactctgtcctgtgagaataagattatctccttcaaggagatgaacccccctgataacatcaaagacactaaatcggacatcattttctttcagaggagcgtgccaggccacgacaacaagatgcagttcgagagttctagttacgagggatatttcctggcctgcgaaaaagaacgtgacctcttcaagctaatcctgaagaaggaggacgagctcggtgacaggagcatcatgtttactgtccaaaacgaggactga

SEQ ID NO: 224 - DNA, 3213 bp, Artificial Sequence
  G12-2R1_134_ECD_T2A_hIL-18
atggctcgcctcggaaattgttccctgacctgggcagcattgatcattctcctgctaccagggagtctcgaagaatgtggccatatctctgtaagcgcccctattgtgcacttgggagacccaatcacagcatcatgcatcatcaagcagaattgctctcatctcgatccagagccacagatactctgggagctgggcgcagaactgcaacccggagggcgccagcagagactgagtgatggaacacaggagagcattatcactttacctcatctgaaccacacccaggcctttcttagctgctgtttgaactggggcaacagtctccagattctggaccaggtggagctgagggcgggttacccgcccgccattccacacaatttgtcctgtctcatgaacctgacaacaagtagcctgatatgccagtgggagccagggccggagacccatctcccaactagtttcacgctcaagagcttcaagtcagaggggaactgccagacacaaggggacagcattctcgactgtgtgcccaaggacgggcagagtcactgttgcattccagacaagcatctgctgctgtatcagaacatgggcatttgggttcaggccgaaaatgccctcggtacttccatgagtccacagttgtgtcttgaccccatggatgtggtgaagctggaaccgccaatgctgaggaccatggatccctcacctgaggctgctccgccacaggcagggtgtctgcagctgtgctgggagccctggcagcctggattgcacataaaccaaaaatgtgagctgaggcacaagccccagagaggtgaggcgagttgggccctggtagggcctcttcctctagaagccctgcagtatgaattgtgtggattacttcccgcaaccgcctacactcttcagatcaggtgtatccggtggcctctgcccggccactggagcgactggtctccgagcctcgaattgagaacaacagaacgggctcctacggtgcgtctggatacatggtggaggcagcgacagttggacccgcgtacagtgcagctgttctggaagcccgtacctctggaagaggatagtggtcggattcagggatatgtggtctcttggcggccttccggtcaggctggggctatccttcccttatgcaacactacagagctcagctgtacctttcatttacccagcgaagcccaggaagtggctctcgtcgcctataattccgctggaacgtcaagacccacccctgtcgtgttctctgagtctcggggacctgccctaaccaggctccatgcaatggcacgcgatcctcacagtctgtgggttggctgggaaccaccaaatccctggcctcagggctatgtcatcgaatggggccttggccccccatcagcctccaatagcaacaagacctggagaatggagcaaaatgggagagcaaccgggttcctgctgaaggagaacatccgaccgttccagctttatgaaattattgtaactccactgtaccaggacacaatgggaccatctcagcatgtctacgcttattcccaggagatggcgcctagtcatgctcctgaattacacttgaagcacatagggaaaacttgggcccaattagagtgggtcccggagcctcccgagctggggaaaagcccattgacccattacacgatattttggaccaatgctcagaaccaatccttttctgccatcctgaatgcctcatctcgaggctttgtgctgcatggcctagagcctgcatcactctaccacattcacctgatggccgccagccaggcgggtgccacaaactccacagttctcactttaatgactctaaccccagagggcagtgaactgcacatcatattagggttattcggattgctgcttcttttgacttgcctctgtggcactgcgtggctgtgctgctccccgaaccgaaagaatcctctctggcctagcgttcccgatcctgctcacagttcactaggatcatgggtgccaaccatcatggaagaggatgccttccagctaccaggactgggtacgccgcccataaccaaattaacggtccttgaagaggatgagaagaaaccagttccatgggaaagccactcttccaatcatagcctcacctcttgcttcaccaaccaagggtatttttttttccacctgcccgacgctctggaaattgaggcctgccaagtgtatttcacctacgacccttactctgaagaagatccggcaggcgacctacccactcatgatggctacctgccgtcaaacatcgacgacttaccgagtggaggaccctccccaccttctacagcccctggtggctccggtgcaggggaggaaaggatgcctccctcactgcaggagagagtccccagagactgggatcctcagccacttggaccacccacacctggtgtgcctgaccttgtggattttcagccccctcctgagcttgttcttcgagaagcaggtgaggaggttcctgatgcaggcccacgtgagggggtctcctttccctggtccaggcctcctggccaaggagagttccgcgctctgaatgcacgcttgcctctgaacacagatgcttacctgagcctacaagagttacaaggccaggatccgacacacctggtgggatctggcgagggtagaggcagcctcctaacttgtggcgacgtggaggagaaccccggtccctacttcggtaagctggagtctaaactgtcggtgatacggaacctgaacgaccaggtcctgttcattgaccagggtaaccgcccgctgttcgaggacatgactgactccgattgccgcgacaatgccccccgcacgatcttcattatctccatgtacaaggatagccaaccccgtggtatggccgtgaccattagcgtcaaatgtgagaagatctctactctgtcctgtgagaataagattatctccttcaaggagatgaacccccctgataacatcaaagacactaaatcggacatcattttctttcagaggagcgtgccaggccacgacaacaagatgcagttcgagagttctagttacgagggatatttcctggcctgcgaaaaagaacgtgacctcttcaagctaatcctgaagaaggaggacgagctcggtgacaggagcatcatgtttactgtccaaaacgaggactgatga

SEQ ID NO: 225 - DNA, 2673 bp, Artificial Sequence
  G12/2R-1 134 ECD
atggctcgcctcggaaattgttccctgacctgggcagcattgatcattctcctgctaccagggagtctcgaagaatgtggccatatctctgtaagcgcccctattgtgcacttgggagacccaatcacagcatcatgcatcatcaagcagaattgctctcatctcgatccagagccacagatactctgggagctgggcgcagaactgcaacccggagggcgccagcagagactgagtgatggaacacaggagagcattatcactttacctcatctgaaccacacccaggcctttcttagctgctgtttgaactggggcaacagtctccagattctggaccaggtggagctgagggcgggttacccgcccgccattccacacaatttgtcctgtctcatgaacctgacaacaagtagcctgatatgccagtgggagccagggccggagacccatctcccaactagtttcacgctcaagagcttcaagtcagaggggaactgccagacacaaggggacagcattctcgactgtgtgcccaaggacgggcagagtcactgttgcattccagacaagcatctgctgctgtatcagaacatgggcatttgggttcaggccgaaaatgccctcggtacttccatgagtccacagttgtgtcttgaccccatggatgtggtgaagctggaaccgccaatgctgaggaccatggatccctcacctgaggctgctccgccacaggcagggtgtctgcagctgtgctgggagccctggcagcctggattgcacataaaccaaaaatgtgagctgaggcacaagccccagagaggtgaggcgagttgggccctggtagggcctcttcctctagaagccctgcagtatgaattgtgtggattacttcccgcaaccgcctacactcttcagatcaggtgtatccggtggcctctgcccggccactggagcgactggtctccgagcctcgaattgagaacaacagaacgggctcctacggtgcgtctggatacatggtggaggcagcgacagttggacccgcgtacagtgcagctgttctggaagcccgtacctctggaagaggatagtggtcggattcagggatatgtggtctcttggcggccttccggtcaggctggggctatccttcccttatgcaacactacagagctcagctgtacctttcatttacccagcgaagcccaggaagtggctctcgtcgcctataattccgctggaacgtcaagacccacccctgtcgtgttctctgagtctcggggacctgccctaaccaggctccatgcaatggcacgcgatcctcacagtctgtgggttggctgggaaccaccaaatccctggcctcagggctatgtcatcgaatggggccttggccccccatcagcctccaatagcaacaagacctggagaatggagcaaaatgggagagcaaccgggttcctgctgaaggagaacatccgaccgttccagctttatgaaattattgtaactccactgtaccaggacacaatgggaccatctcagcatgtctacgcttattcccaggagatggcgcctagtcatgctcctgaattacacttgaagcacatagggaaaacttgggcccaattagagtgggtcccggagcctcccgagctggggaaaagcccattgacccattacacgatattttggaccaatgctcagaaccaatccttttctgccatcctgaatgcctcatctcgaggctttgtgctgcatggcctagagcctgcatcactctaccacattcacctgatggccgccagccaggcgggtgccacaaactccacagttctcactttaatgactctaaccccagagggcagtgaactgcacatcatattagggttattcggattgctgcttcttttgacttgcctctgtggcactgcgtggctgtgctgctccccgaaccgaaagaatcctctctggcctagcgttcccgatcctgctcacagttcactaggatcatgggtgccaaccatcatggaagaggatgccttccagctaccaggactgggtacgccgcccataaccaaattaacggtccttgaagaggatgagaagaaaccagttccatgggaaagccactcttccaatcatagcctcacctcttgcttcaccaaccaagggtatttttttttccacctgcccgacgctctggaaattgaggcctgccaagtgtatttcacctacgacccttactctgaagaagatccggcaggcgacctacccactcatgatggctacctgccgtcaaacatcgacgacttaccgagtggaggaccctccccaccttctacagcccctggtggctccggtgcaggggaggaaaggatgcctccctcactgcaggagagagtccccagagactgggatcctcagccacttggaccacccacacctggtgtgcctgaccttgtggattttcagccccctcctgagcttgttcttcgagaagcaggtgaggaggttcctgatgcaggcccacgtgagggggtctcctttccctggtccaggcctcctggccaaggagagttccgcgctctgaatgcacgcttgcctctgaacacagatgcttacctgagcctacaagagttacaaggccaggatccgacacacctggtg

SEQ ID NO: 226 - DNA, 63 bp, Artificial Sequence
  T2A
ggatctggcgagggtagaggcagcctcctaacttgtggcgacgtggaggagaaccccggtccc

SEQ ID NO: 227 - protein, 10 aa, Artificial Sequence
  Myc epitope
EQKLISEEDL

SEQ ID NO: 228 - protein, 8 aa, Artificial Sequence
  Flag epitope
DYKDDDDK

SEQ ID NO: 229 - protein, 175 aa, Artificial Sequence; identical to SEQ ID NO: 82
  Human G-CSF recombinantly produced in E. coli

SEQ ID NO: 230 - protein, 39 aa, Artificial Sequence
  TEV-cleavable Twin Strep tag
  VARIANT (9)..(9): X can be Q or G
AAAENLYFXSAWSHPQFEKGGGSGGGSGGSAWSHPQFEK

SEQ ID NO: 231 - protein, 19 aa, Artificial Sequence
  TEV-cleavable Hexahistidine
  VARIANT (16)..(16): X can be Q or G
HHHHHHSSGRENLYFXSMG

SEQ ID NO: 232 - protein, 9 aa, Artificial Sequence
  G-CSFR
LWPSVPDPA

SEQ ID NO: 233 - protein, 9 aa, Artificial Sequence
  gp130
IWPNVPDPS

SEQ ID NO: 234 - protein, 9 aa, Artificial Sequence
  IL-2R Beta
LKCNTPDPS

SEQ ID NO: 235 - protein, 9 aa, Artificial Sequence
  IL-21R
KIWAVPSPE

SEQ ID NO: 236 - protein, 9 aa, Artificial Sequence
  IL-12R beta2
CSREIPDPA

SEQ ID NO: 237 - protein, 9 aa, Artificial Sequence
  IL-7R alfa
VWPSLPDHK